(19) United States
(12) Patent Application Publication
(10) Pub. No.: US 2024/0327380 A1
(43) Pub. Date: Oct. 3, 2024

(54) BTK INHIBITORS

(71) Applicant: BIOGEN MA INC., Cambridge, MA (US)

(72) Inventors: Brian T. Hopkins, Newton, MA (US); Isaac Marx, Arlington, MA (US); Jürgen Schulz, Bonn (DE); Harold George Vandeveer, Stoneham, MA (US); Robin Prince, Sharon, MA (US); Marta Nevalainen, Holliston, MA (US); TeYu Chen, Charlestown, MA (US); Zain Yousaf, Quincy, MA (US); Martin Himmelbauer, Cambridge, MA (US); Vatee Pattaropong, Bedford, MA (US); John Howard Jones, Framingham, MA (US); Rab Gilfillan, Ashland, MA (US); Edward Yin Shiang Lin, Ashland, MA (US); Felix Gonzalez Lopez de Turiso, Cambridge, MA (US); Bin Ma, Arlington, MA (US)

(73) Assignee: BIOGEN MA INC., Cambridge, MA (US)

(21) Appl. No.: 18/019,540

(22) PCT Filed: Aug. 5, 2021
(86) PCT No.: PCT/US2021/044829

§ 371 Date: Feb. 3, 2023

Related U.S. Application Data
(60) Provisional application No. 63/189,476, filed on May 17, 2021.
(60) Provisional application No. 63/063,188, filed on Aug. 7, 2020.

(51) Int. Cl. C07D 401/14; A61K 31/4439; A61K 31/4545; A61K 31/496; A61K 31/506; A61K 31/5377; A61K 31/55; A61K 31/553; C07D 401/12; C07D 403/12; C07D 413/12; C07D 413/14; C07D 471/04; C07D 471/08; C07D 487/10

(52) U.S. Cl. CPC C07D 401/14; A61K 31/4439; A61K 31/4545; A61K 31/496; A61K 31/506; A61K 31/5377; A61K 31/55; A61K 31/553; C07D 401/12; C07D 403/12; C07D 413/12; C07D 413/14; C07D 471/04; C07D 471/08; C07D 487/10

(57) ABSTRACT

Provided are compounds of Formula (I): or pharmaceutically acceptable salts thereof, wherein R¹, R², R³, R⁴, R⁵, R⁷, R⁸, R⁹, A¹, A², Q¹, Q², Q³, A, Z, m and n are as defined herein; pharmaceutical compositions comprising said compounds or pharmaceutically acceptable salts thereof, and pharmaceutically acceptable excipients; and methods of treating a disorder responsive to inhibition of Bruton's tyrosine kinase using said compounds, or pharmaceutically acceptable salts thereof, or said pharmaceutical compositions.

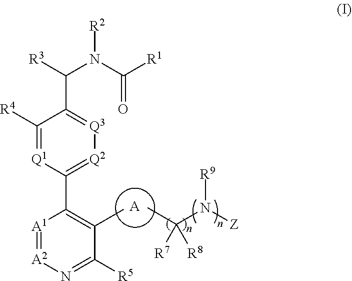

### RELATED APPLICATIONS

This application claims the benefit of the filing date, under 35 U.S.C. § 119(e), of U.S. Provisional Application No. 63/063,188, filed on Aug. 7, 2020 and U.S. Provisional Application No. 63/189,476, filed on May 17, 2021, the entire contents of each of above-referenced applications are incorporated herein by reference.

### TECHNICAL FIELD

Provided are certain agents that inhibit Bruton's tyrosine kinase (Btk), and methods of making and using such agents.

### BACKGROUND

Protein kinases are a large multigene family consisting of more than 500 proteins which play a critical role in the development and treatment of a number of human diseases in oncology, neurology and immunology. The Tec kinases are non-receptor tyrosine kinases which consists of five members (Tec (tyrosine kinase expressed in hepatocellular carcinoma), Btk (Bruton's tyrosine kinase), Itk (interleukin-2 (IL-2)-inducible T-cell kinase; also known as Emt or Tsk), Rlk (resting lymphocyte kinase; also known as Txk) and Bmx (bone-marrow tyrosine kinase gene on chromosome X; also known as Etk)) and are primarily expressed in haematopoietic cells, although expression of Bmx and Tec has been detected in endothelial and liver cells. Tec kinases (Itk, Rlk and Tec) are expressed in T cell and are all activated downstream of the T-cell receptor (TCR). Btk is a downstream mediator of B cell receptor (BCR) signaling which is involved in regulating B cell activation, proliferation, and differentiation. More specifically, Btk contains a PH domain that binds phosphatidylinositol (3,4,5)-trisphosphate (PIP3). PIP3 binding induces Btk to phosphorylate phospholipase C (PLCy), which in turn hydrolyzes PIP2 to produce two secondary messengers, inositol triphosphate (IP3) and diacylglycerol (DAG), which activate protein kinase PKC, which then induces additional B-cell signaling. Mutations that disable Btk enzymatic activity result in XLA syndrome (X-linked agammaglobulinemia), a primary immunodeficiency. Given the critical roles which Tec kinases play in both B-cell and T-cell signaling, Tec kinases are targets of interest for autoimmune disorders.

Consequently, there is a great need in the art for effective inhibitors of Btk.

### SUMMARY

A first embodiment of the invention is a compound of Formula (I):

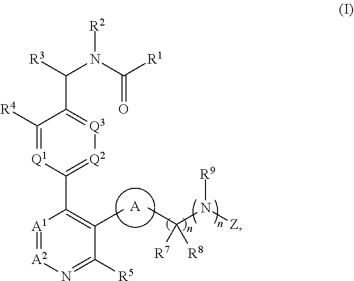

or a pharmaceutically acceptable salt thereof, wherein:
one of A¹ and A² is C—R^{6A}, and the other of A¹ and A² is C—R^{6A} or N;
Q¹ is selected from C—R⁶ and N;
Q² is selected from C—R⁶ and N;
Q³ is selected from C—R⁶ and N;
wherein at most one of Q¹, Q², and Q³ is N;
ring A is a 4- to 8-membered monocyclic saturated or partially saturated heterocyclyl, substituted with one or more R¹¹;
n is 0 or 1;
mis 0 or 1;
R¹ is selected from —N(R^{1a})₂, phenyl, 3- to 7-membered saturated or partially unsaturated monocyclic carbocyclyl, 3- to 7-membered saturated or partially unsaturated monocyclic heterocyclyl, 5- to 6-membered heteroaryl, 7- to 10-membered saturated or partially unsaturated bicyclic carbocyclyl, 7- to 10-membered saturated or partially unsaturated bicyclic heterocyclyl, 8- to 10-membered bicyclic heteroaryl, and 9- to 10-membered bicyclic aryl, wherein the phenyl, 3- to 7-membered saturated or partially unsaturated monocyclic carbocyclyl, 3- to 7-membered saturated or partially unsaturated monocyclic heterocyclyl, 5- to 6-membered heteroaryl, 7- to 10-membered saturated or partially unsaturated bicyclic carbocyclyl, 7- to 10-membered saturated or partially unsaturated bicyclic heterocyclyl, and 9- to 10-membered bicyclic aryl represented by R¹ are each optionally substituted with one or more R¹²;
R^{1a}, for each occurrence, is independently selected from H, C₁₋₆ alkyl, C₂₋₆ alkenyl, C₂₋₆ alkynyl, phenyl, 3- to 7-membered saturated or partially unsaturated carbocyclyl ring, 3- to 7-membered saturated or partially unsaturated monocyclic heterocyclyl, and 5- to 6-membered heteroaryl, wherein the C₁₋₆ alkyl, C₂₋₆ alkenyl, C₂₋₆ alkynyl, phenyl, 3- to 7-membered saturated or partially unsaturated carbocyclyl ring, 3- to 7-membered saturated or partially unsaturated monocyclic heterocyclyl, and 5- to 6-membered heteroaryl represented by R^{1a} are each optionally substituted with one or more R¹²;
or two R^{1a} groups on the same nitrogen are taken together with their intervening atoms to form a ring selected from 3- to 7-membered saturated or partially unsaturated monocyclic heterocyclyl and 5- to 6-membered heteroaryl, wherein the ring is optionally substituted with one or more R¹²;
R¹², for each occurrence, is independently selected from halogen, —OR^{12a}, —S(O)₂R^{12a}, —CN, C₁₋₆ alkyl, C₂₋₆ alkenyl, C₂₋₆ alkynyl, 3- to 7-membered saturated or partially unsaturated monocyclic carbocyclyl, and 4- to 6-membered saturated or partially unsaturated monocyclic heterocyclyl; wherein the C₁₋₆ alkyl, C₂₋₆ alkenyl, C₂₋₆ alkynyl, 3- to 7-membered saturated or partially unsaturated monocyclic carbocyclyl, and 4- to 6-membered saturated or partially unsaturated monocyclic heterocyclyl represented by R¹² are each optionally substituted with one or more R¹⁵;
R^{12a} is C₁₋₆ alkyl optionally substituted with one or more halogen;
R¹⁵, for each occurrence, is independently selected from halogen, C₁₋₆ alkyl, C₁₋₆ haloalkyl, —CN, and —OR^{5a};
R^{15a} is C₁₋₆ alkyl;
R² is H, C₁₋₆ alkyl, C₂₋₆ alkenyl, and C₂₋₆ alkynyl,
or R¹ and R², together with their intervening atoms, form a Ring B selected from 3- to 7-membered saturated or partially unsaturated monocyclic heterocyclyl, 7- to 10-membered saturated or partially unsaturated bicyclic heterocyclyl, and 8- to 10-membered bicyclic heteroaryl, wherein Ring B is optionally substituted with one or more R¹⁰⁰;
R¹⁰⁰, for each occurrence, is independently selected from C₁₋₆ alkyl, C₂₋₆ alkenyl, C₂₋₆ alkynyl, 3- to 7-membered saturated or partially unsaturated monocyclic carbocyclyl, 4- to 6-membered saturated or partially unsaturated monocyclic heterocyclyl and halogen; wherein the C₁₋₆ alkyl, C₂₋₆ alkenyl, C₂₋₆ alkynyl, 3- to 7-membered saturated or partially unsaturated monocyclic carbocyclyl, and saturated or partially unsaturated 4- to 6-membered monocyclic heterocyclyl represented by R¹⁰⁰ are each optionally substituted with one or more R¹⁵⁰;
R¹⁵⁰, for each occurrence, is independently selected from halogen and —OR^{150a};
R^{150a} is C₁₋₆ alkyl;
R³ is selected from H, halogen, —C(O)N(R^{3a})₂, —C(O)OR^{3a}, —C(O)R^{3a}, C₁₋₆ alkyl, C₂₋₆ alkenyl, and C₂₋₆ alkynyl, wherein the C₁₋₆ alkyl, C₂₋₆ alkenyl, and C₂₋₆ alkynyl represented by R³ are each optionally substituted with one or more substituents selected from halogen and hydroxyl;
R^{3a}, for each occurrence, is independently selected from H, C₁₋₆ alkyl, C₂₋₆ alkenyl, C₂₋₆ alkynyl, phenyl, 3- to 7-membered saturated or partially unsaturated carbocyclyl ring, 3- to 7-membered saturated or partially unsaturated monocyclic heterocyclyl, or 5- to 6-membered heteroaryl, wherein C₁₋₆ alkyl, C₂₋₆ alkenyl, C₂₋₆ alkynyl, phenyl, 3- to 7-membered saturated or partially unsaturated carbocyclyl ring, 3- to 7-membered saturated or partially unsaturated monocyclic heterocyclyl, and 5- to 6-membered heteroaryl are optionally substituted with one or more R³⁰;
or two R^{3a} groups on the same nitrogen are taken together with their intervening atoms to form a ring selected from 3- to 7-membered saturated or partially unsaturated monocyclic heterocyclyl and 5- to 6-membered heteroaryl, wherein said ring is optionally substituted with one or more R³⁰;
R³⁰, for each occurrence, is independently selected from halogen, —OR^{30a}, —N(R^{30a})₂, —C(O)N(R^{30a}), —C(O)₂R^{30a}, oxo, C₁₋₆ alkyl, C₂₋₆ alkenyl, C₂₋₆ alkynyl, 3- to 7-membered saturated or partially unsaturated monocyclic carbocyclyl, and 4- to 6-membered saturated or partially unsaturated monocyclic heterocyclyl;
R^{30a} is H or C₁₋₆ alkyl;
R⁴ is selected from H, halogen, —NO₂, —CN, —OR^{4a}, —SR^{4a}, —N(R^{4a})₂, —C(O)R^{4a}, —C(O)OR^{4a}, —S(O)R^{4a}, —S(O)₂R^{4a}, —C(O)N(R^{4a})₂, —SO₂N(R^{4a})₂, —OC(O)R^{4a}, —N(R^{4a})C(O)R^{4a}, —N(R^{4a})C(O)OR^{4a}, —N(R^{4a})SO₂R^{4a}, —OC(O)N(R^{4a})₂, C₁₋₆ alkyl, C₂₋₆ alkenyl and C₂₋₆ alkynyl, wherein the C₁₋₆ alkyl, C₂₋₆ alkenyl and C₂₋₆ alkynyl are each optionally substituted with one or more R⁴⁰;
R^{4a} is H, C₁₋₆ alkyl, C₂₋₆ alkenyl, C₂₋₆alkynyl, phenyl, 3- to 8-membered saturated or partially unsaturated carbocyclyl ring, 3- to 7-membered saturated or partially unsaturated monocyclic heterocyclyl, and 5- to 6-membered heteroaryl, wherein the C₁₋₆ alkyl, C₂₋₆ alkenyl, C₂₋₆ alkynyl, phenyl, 3- to 8-membered saturated or partially unsaturated carbocyclyl ring, 3- to 7-membered saturated or partially unsaturated monocyclic heterocyclyl, and 5- to 6-membered heteroaryl represented by R^{4a} are each optionally substituted with one or more R⁴⁰;
or two R^{4a} groups on the same nitrogen are taken together with their intervening atoms to form a ring selected from 3- to 7-membered saturated or partially unsaturated monocyclic heterocyclyl and 5- to 6-membered heteroaryl, wherein said ring is optionally substituted with one or more R⁴⁰;
R⁴⁰, for each occurrence, is independently selected from halogen, —OR^{40a}, —N(R^{40a})₂, —C(O)N(R^{40a})₂, —C(O)₂R^{40a}, oxo, C₁₋₆ alkyl, C₂₋₆ alkenyl, C₂₋₆ alkynyl, 3- to 7-membered monocyclic carbocyclyl, and 4- to 6-membered monocyclic heterocyclyl; wherein the C₁₋₆ alkyl, C₂₋₆ alkenyl, C₂₋₆ alkynyl, 3- to 7-membered monocyclic carbocyclyl, and 4- to 6-membered monocyclic heterocyclyl represented by R⁴⁰ are each optionally substituted with one or more R⁴⁵;
R^{40a} is H, C₁₋₆ alkyl, C₂₋₆ alkenyl, C₂₋₆ alkynyl, 3- to 7-membered monocyclic carbocyclyl, and 4- to 6-membered monocyclic heterocyclyl, wherein the C₁₋₆ alkyl, C₂₋₆ alkenyl, C₂₋₆ alkynyl, 3- to 7-membered monocyclic carbocyclyl, and 4- to 6-membered monocyclic heterocyclyl are each optionally substituted with one or more R⁴⁵;
R⁴⁵, for each occurrence, is independently selected from C₁₋₆ alkyl, halogen and —OR^{45a};
R^{45a} is H or C₁₋₆ alkyl;
or R³ and R⁴, together with their intervening atoms, form a Ring C, wherein Ring C is selected from 5- to 7-membered monocyclic carbocycle and 5- to 7-membered monocyclic heterocycle, wherein Ring C is optionally substituted with R³⁰⁰;
R³⁰⁰, for each occurrence, is independently selected from C₁₋₆ alkyl, C₂₋₆ alkenyl, C₂₋₆ alkynyl, 3- to 7-membered monocyclic carbocyclyl, 4- to 6-membered monocyclic heterocyclyl, halogen, —C(O)R^{300a}, —OR^{300a}, and —S(O)₂R^{300a}; wherein the C₁₋₆ alkyl, C₂₋₆ alkenyl, C₂₋₆ alkynyl, 3- to 7-membered monocyclic carbocyclyl, and 4- to 6-membered monocyclic heterocyclyl represented by R³⁰⁰ are each optionally substituted with one or more R³⁵⁰;
R^{300a} is selected from H, C₁₋₆ alkyl, C₂₋₆ alkenyl, C₂₋₆ alkynyl, 3- to 7-membered monocyclic carbocyclyl, and 4- to 6-membered monocyclic heterocyclyl, wherein the C₁₋₆ alkyl, C₂₋₆ alkenyl, C₂₋₆ alkynyl, 3- to 7-membered monocyclic carbocyclyl, and 4- to 6-membered monocyclic heterocyclyl represented by R^{300a} are each optionally substituted with one or more R³⁵⁰;
R³⁵⁰, for each occurrence, is independently selected from C₁₋₆ alkyl, halogen, —CN, —C(O)R^{350a}, —C(O)N(R^{350a})₂, —C(R^{350a})₂N(R^{350a})₂, and —OR^{350a};
R^{350a}, for each occurrence, is independently H or C₁₋₆ alkyl optionally substituted with one to three halogen;
R⁵ is selected from H, —NHR^{5s}, or —NHC(O)R^{5s};
R^{5a} is H or C₁₋₆alkyl;
R⁶ and R^{6A}, for each occurrence, are independently selected from H, C₁₋₆ alkyl, C₂₋₆ alkenyl, C₂₋₆ alkynyl, halogen, —NO₂, —CN, —OR^{6a}, —SR^{6a}, —N(R^{6a})₂, —C(O)R^{6a}, —C(O)OR^{6a}, —S(O)R^{6a}, —S(O)₂R^{6a}, —C(O)N(R^{6a})₂, —SO₂N(R^{6a})₂, —OC(O)R^{6a}, —N(R^{6a})C(O)R^{6a}, —N(R⁶a)C(O)OR^{6a}, —N(R^{6a})SO₂R^{6a}, and —OC(O)N(R^{6a});
R^{6a} is H, C₁₋₆ alkyl, C₂₋₆ alkenyl, C₂₋₆ alkynyl, 3- to 7-membered monocyclic carbocyclyl, and 4- to 6-membered monocyclic heterocyclyl, wherein the C₁₋₆ alkyl, C₂₋₆ alkenyl, C₂₋₆ alkynyl, 3- to 6-membered monocyclic carbocyclyl and 4- to 6-membered monocyclic heterocyclyl represented by R^{6a} are each optionally substituted with one or more R⁶⁰;
R⁶⁰, for each occurrence, is independently selected from halogen, —OR^{60a}, C₁₋₆ alkyl, C₂₋₆ alkenyl, C₂₋₆ alkynyl, 3- to 7-membered monocyclic carbocyclyl, and 4- to 6-membered monocyclic heterocyclyl; wherein the C₁₋₆ alkyl, C₂₋₆ alkenyl, C₂₋₆ alkynyl, 3- to 7-membered monocyclic carbocyclyl and 4- to 6-membered monocyclic heterocyclyl represented by R⁶⁰ are optionally substituted with one or more R⁶⁵;
R^{60a} is H, C₁₋₆ alkyl, C₂₋₆ alkenyl, C₂₋₆ alkynyl, 3- to 7-membered monocyclic carbocyclyl, and 4- to 6-membered monocyclic heterocyclyl, wherein the C₁₋₆ alkyl, C₂₋₆ alkenyl, C₂₋₆ alkynyl, 3- to 7-membered monocyclic carbocyclyl, and 4- to 6-membered monocyclic heterocyclyl represented by R^{60a} are each optionally substituted with one or more R⁶⁵;
R⁶⁵, for each occurrence, is independently selected from C₁₋₆ alkyl, halogen and —OR^{65a};
R^{65a} is H or C₁₋₆ alkyl;
R⁷ and R⁸ are each independently H or C₁₋₆ alkyl optionally substituted with one or more substituents independently selected from halogen and C₁₋₆alkoxy;
R⁹ is H, C₁₋₆ alkyl or C₃₋₆cycloalkyl, wherein the C₁₋₆alkyl is optionally substituted with one or more substituents independently selected from halogen and C₁₋₆alkoxy and the C₃₋₆cycloalkyl is optionally substituted with one or more substituents independently selected from C₁₋₆alkyl, halogen, C₁₋₆haloalkyl and C₁₋₆alkoxy;
or when m is 1, R⁹ and one of R¹¹ on ring A together with their intervening atoms form a 4- to 7-membered monocyclic saturated or partially saturated heterocyclyl, which is optionally substituted with one or more substituents independently selected from halogen, —CN, —OH, C₁₋₆ alkyl and C₁₋₆ alkoxy;
Z is —C(═O)R¹⁰, —SO₂R¹⁰, or —CN;
R¹⁰ is C₂₋₆ alkenyl, C₂₋₆ alkynyl, C₂₋₆ alkylenyl oxide, or C₄₋₇ cycloalkenyl, wherein the C₂₋₆ alkenyl represented by R¹⁰ is optionally substituted with one or more substituents independently selected from halo, C₁₋₆ alkyl, C₁₋₆ alkoxy and —NR^{10a}R^{10b}, the C₂₋₆ alkynyl represented by R²⁰⁰ is optionally substituted by one or more substituents independently selected from C₁₋₆ alkyl and C₁₋₆ alkoxy, and the C₂₋₆ alkylenyl oxide represented by R¹⁰ is optionally substituted by one or more C₁₋₆ alkyl;
R^{10a} and R^{10b} are each independently H or C₁₋₃ alkyl; or R^{10a} and R^{10b} together with the nitrogen atom from they are they are attached form a 4- to 7-membered monocyclic saturated heterocyclyl optionally substituted with one or more substituents independently selected from halo and C₁₋₆ alkyl; and
R¹¹, for each occurrence, is independently selected from H, halogen, —CN, —OH, C₁₋₆ alkyl, C₁₋₆ haloalkyl and C₁₋₆ alkoxy, or two R¹¹ together with the same carbon atom from which they are attached form a —C(═O)— group.

The present invention also provides a pharmaceutical composition comprising at least one compound described herein, or a pharmaceutically acceptable salt thereof, and at least one pharmaceutically acceptable excipient.

In one embodiment, the invention is a method of treating a disorder responsive to inhibition of Btk in a subject comprising administering to the subject an effective amount of at least one compound described herein, or a pharmaceutically acceptable salt thereof, or a pharmaceutical composition described herein.

The present invention also includes the use of at least one compound described herein, or a pharmaceutically acceptable salt thereof, for the manufacture of a medicament for the treatment of a disorder responsive to inhibition of Btk. Also provided is a compound described herein, or a pharmaceutically acceptable salt thereof, or a pharmaceutical composition described herein for use in treating a disorder responsive to inhibition of Btk.

Other features or advantages will be apparent from the following detailed description of several embodiments, and also from the appended claims.

### DETAILED DESCRIPTION

The compounds or pharmaceutically acceptable salts thereof as described herein, can have activity as Btk modulators. In particular, compounds or pharmaceutically acceptable salts thereof as described herein, can be Btk inhibitors.

In a second embodiment, a compound of the present invention is represented by Formula (I), or a pharmaceutically acceptable salt thereof, wherein
R¹⁵, for each occurrence, is independently selected from halogen and —OR^{15a};
R¹⁰ is C₂₋₆ alkenyl, C₂₋₆ alkynyl or C₂₋₆ alkylenyl oxide, wherein the C₂₋₆ alkenyl represented by R¹⁰ is optionally substituted with one or more substituents independently selected from C₁₋₆ alkyl, C₁₋₆ alkoxy and —NR^{10a}R^{10b}, the C₂₋₆ alkynyl represented by R¹⁰ is optionally substituted by one or more substituents independently selected from C₁₋₆ alkyl and C₁₋₆ alkoxy, and the C₂₋₆ alkylenyl oxide represented by R¹⁰ is optionally substituted by one or more C₁₋₆ alkyl;
R^{10a} and R^{10b} are each independently H or C₁₋₃ alkyl; and
R¹¹, for each occurrence, is independently selected H, halogen, —CN, —OH, C₁₋₆ alkyl and C₁₋₆ alkoxy, or two R¹¹ together with the same carbon atom from which they are attached form a —C(═O)— group, wherein the other variables are defined as in the first embodiment.

In a third embodiment, a compound of the present invention is represented by Formula (I), or a pharmaceutically acceptable salt thereof, wherein Q¹, Q² and Q³ are C—R⁶; and the definitions for the other variables are as defined in the first or second embodiment.

In a fourth embodiment, a compound of the present invention is represented by Formula (I), or a pharmaceutically acceptable salt thereof, wherein A¹ is N and A² is C—R^{6A}; and the definitions for the other variables are as defined in the first, second, or third embodiment.

In a fifth embodiment, a compound of the present invention is represented by Formula (I), or a pharmaceutically acceptable salt thereof, wherein A¹ and A² are both C—R^{6A}; and the definitions for the other variables are as defined in the first, second, or third embodiment.

In a sixth embodiment, a compound of the present invention is represented by Formula (IIA) or Formula (IIB):

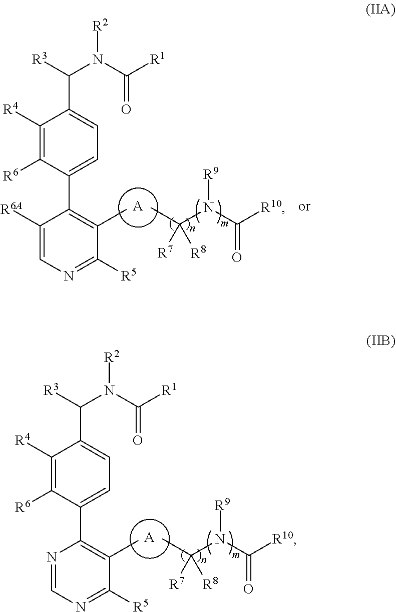

or a pharmaceutically acceptable salt thereof; and the definitions for the variables are as defined in the first, second, third, fourth, or fifth embodiment.

In a seventh embodiment, a compound of the present invention is represented by Formula (I), (IIA), or (IIB), or a pharmaceutically acceptable salt thereof, wherein R¹ is a 3- to 7-membered saturated or partially unsaturated monocyclic heterocyclyl having 1-2 heteroatoms independently selected from O, N and S, or a 5- to 6-membered heteroaryl having 1-4 heteroatoms independently selected from O, N and S, wherein the 3- to 7-membered saturated or partially unsaturated monocyclic heterocyclyl and 5- to 6-membered heteroaryl represented by R¹ are optionally substituted with one or two R¹⁰; and the definitions for the other variables are as defined in the first, second, third, fourth, fifth, or sixth embodiment.

In an eighth embodiment, a compound of the present invention is represented by Formula (I), (IIA), or (IIB), or a pharmaceutically acceptable salt thereof, wherein R¹ is a 5- to 6-membered heteroaryl optionally substituted with one or two R¹⁰; and the definitions for the other variables are as defined in the first, second, third, fourth, fifth, sixth, or seventh embodiment.

In a ninth embodiment, a compound of the present invention is represented by Formula (I), (IIA), or (IIB), or a pharmaceutically acceptable salt thereof, wherein R¹ is a 5-membered heteroaryl selected from pyridinyl, pyrazolyl, imidazolyl, oxazolyl, isoxazolyl, 1,2,3-oxadiazolyl, 1,3,4-oxadiazolyl, 1,2,4-oxadizolyl, 1,2,3-thiadiazolyl, 1,3,4-thiadiazolyl, 1,2,4-thiadiazolyl, 1,2,3-triazolyl, 1,2,4-triazolyl, and tetrazolyl, each of which is optionally substituted with one or two R¹⁰; and the definitions for the other variables are as defined in the first, second, third, fourth, fifth, sixth, or seventh embodiment.

In a tenth embodiment, a compound of the present invention is represented by Formula (I), (IIA), or (IIB), or a pharmaceutically acceptable salt thereof, wherein R¹ is represented by the following formula:

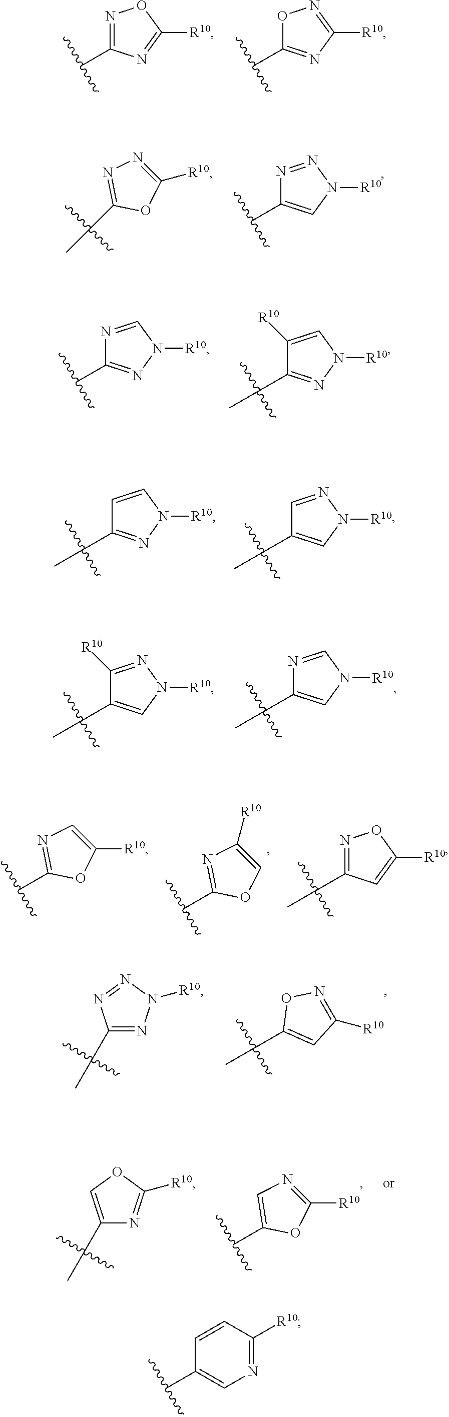

and the definitions for the other variables are as defined in the first, second, third, fourth, fifth, sixth, or seventh embodiment.

In an eleventh embodiment, a compound of the present invention is represented by Formula (I), (IIA), or (IIB), or a pharmaceutically acceptable salt thereof, wherein R is represented by the following formula:

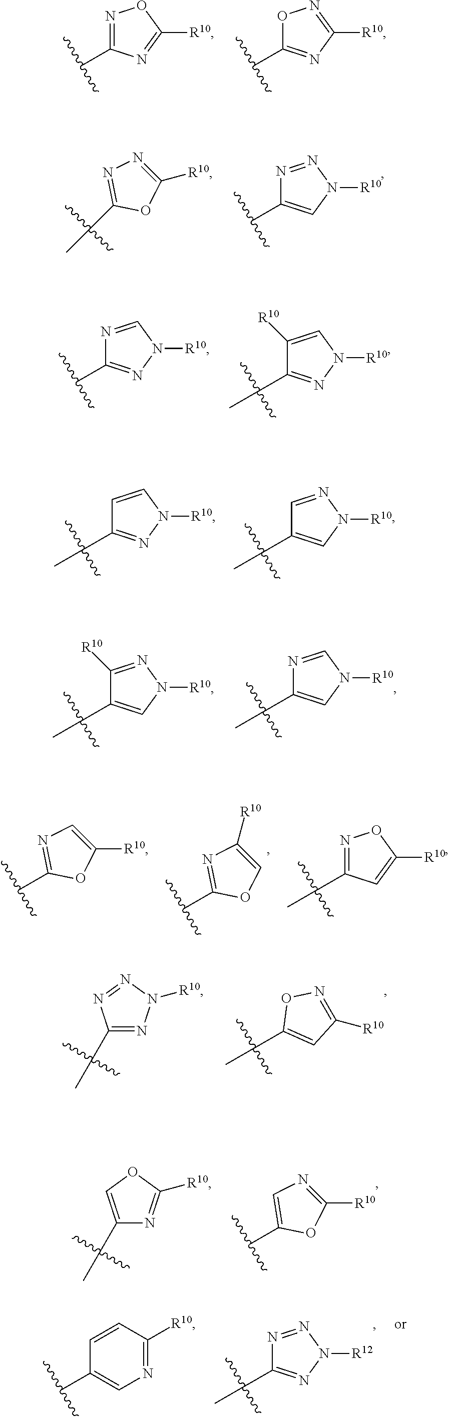

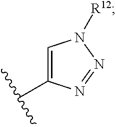

and the definitions for the other variables are as defined in the first, second, third, fourth, fifth, sixth, or seventh embodiment.

In a twelfth embodiment, a compound of the present invention is represented by Formula (I), (IIA), or (IIB), or a pharmaceutically acceptable salt thereof, wherein R¹², for each occurrence, is independently selected from halogen, —OR^{12a}, —S(O)₂R^{12a}, —CN, C₁₋₆ alkyl, C₃₋₆ cycloalkyl, and phenyl; wherein the C₁₋₆ alkyl, C₃₋₆ cycloalkyl, and phenyl represented by R¹² are each optionally substituted with one to three R¹⁵; wherein R^{12a}, for each occurrence, is independently selected from H and C₁₋₃ alkyl; R¹⁵, for each occurrence, is independently selected from halogen and —OR^{15a}; and R^{15a} is H or C₁₋₃ alkyl; and the definitions for the other variables are as defined in the first, second, third, fourth, fifth, sixth, seventh, eighth, ninth, or tenth embodiment.

In a thirteenth embodiment, a compound of the present invention is represented by Formula (I), (IIA), or (IIB), or a pharmaceutically acceptable salt thereof, wherein R¹², for each occurrence, is independently selected from halogen, —OR^{12a}, —S(O)₂R^{12a}, —CN, C₁₋₆ alkyl, C₃₋₆ cycloalkyl, and phenyl; wherein the C₁₋₆ alkyl, C₃₋₆ cycloalkyl, and phenyl represented by R¹² are each optionally substituted with one to three R¹⁵; R^{12a}, for each occurrence, is independently selected from H and C₁₋₃ alkyl; R¹⁵, for each occurrence, is independently selected from C₁₋₆alkyl, halogen, C₁₋₆ haloalkyl, —CN and —OR^{15a}; and R^{15a} is H or C₁₋₃ alkyl; and the definitions for the other variables are as defined in the first, third, fourth, fifth, sixth, seventh, eighth, ninth, tenth, or eleventh embodiment.

In a fourteenth embodiment, a compound of the present invention is represented by Formula (I), (IIA), or (IIB), or a pharmaceutically acceptable salt thereof, wherein R¹², for each occurrence, is independently C₁₋₆ alkyl optionally substituted with one to three halogen or a C₃₋₆cycloalkyl optionally substituted with one or two C₁₋₃alkyl; and the definitions for the other variables are as defined in the first, second, third, fourth, fifth, sixth, seventh, eighth, ninth, tenth, or thirteenth embodiment.

In a fifteenth embodiment, a compound of the present invention is represented by Formula (I), (IIA), or (JIB), or a pharmaceutically acceptable salt thereof, wherein R¹², for each occurrence, is independently —CH₃, —CF₃, or —C(CH₃)₃; and the definitions for the other variables are as defined in the first, second, third, fourth, fifth, sixth, seventh, eighth, ninth, tenth, or twelfth embodiment.

In a sixteenth embodiment, a compound of the present invention is represented by Formula (I), (IIA), or (JIB), or a pharmaceutically acceptable salt thereof, wherein R¹², for each occurrence, is independently —CH₃, —CHF₂, —CF₃, —C(CH₃)₃,

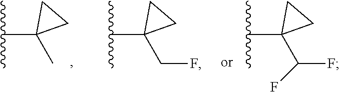

and the definitions for the other variables are as defined in the first, third, fourth, fifth, sixth, seventh, eighth, ninth, tenth, twelfth, or thirteenth embodiment.

In a seventeenth embodiment, a compound of the present invention is represented by Formula (I), (IIA), or (IIB), or a pharmaceutically acceptable salt thereof, wherein R² is H or C₁₋₃ alkyl; and the definitions for the other variables are as defined in the first, second, third, fourth, fifth, sixth, seventh, eighth, ninth, tenth, eleventh, twelfth, thirteenth, fourteenth, fifteenth, or sixteenth embodiment.

In an eighteenth embodiment, a compound of the present invention is represented by Formula (I), (IIA), or (IIB), or a pharmaceutically acceptable salt thereof, wherein R² is H or methyl; and the definitions for the other variables are as defined in the first, second, third, fourth, fifth, sixth, seventh, eighth, ninth, tenth, eleventh, twelfth, or seventeenth embodiment.

In a nineteenth embodiment, a compound of the present invention is represented by Formula (I), (IIA), or (IIB), or a pharmaceutically acceptable salt thereof, wherein R¹ and R², together with their intervening atoms, form a Ring B selected from 3- to 7-membered saturated or partially unsaturated monocyclic heterocyclyl having 1-2 heteroatoms independently selected from O, N and S, 5- to 6-membered heteroaryl having 1-4 heteroatoms independently selected from O, N and S, 7- to 10-membered bicyclic heterocyclyl having 1-4 heteroatoms independently selected from O, N and S, and 8- to 10-membered bicyclic heteroaryl having 1-4 heteroatoms independently selected from O, N and S, wherein Ring B is optionally substituted with one or two R¹⁰⁰; and the definitions for the other variables are as defined in the first, second, third, fourth, fifth, sixth, seventh, eighth, ninth, tenth, eleventh, twelfth, thirteenth, fourteenth, fifteenth, sixteenth, seventeenth, or eighteenth embodiment.

In a twentieth embodiment, a compound of the present invention is represented by Formula (I), (IIA), or (IIB), or a pharmaceutically acceptable salt thereof, wherein Ring B is represented by one of following formulae:

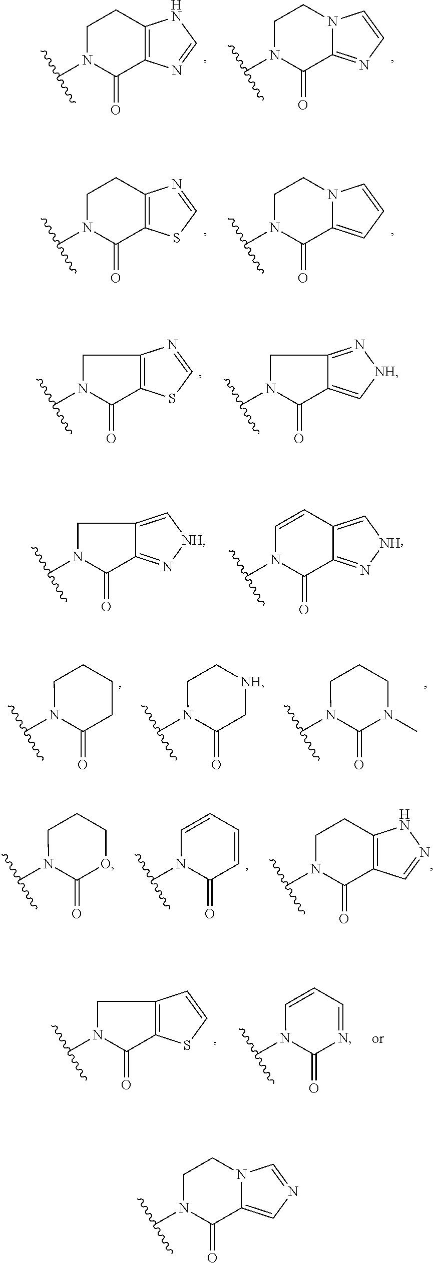

wherein Ring B is optionally substituted with one or two R¹⁰⁰; and the definitions for the other variables are as defined in the first, second, third, fourth, fifth, sixth, seventh, eighth, ninth, tenth, eleventh, twelfth, thirteenth, fourteenth, fifteenth or nineteenth embodiment.

In a twenty-first embodiment, a compound of the present invention is represented by Formula (I), (IIA), or (IIB), or a pharmaceutically acceptable salt thereof, wherein R¹⁰⁰, for each occurrence, is independently selected from C₁₋₆ alkyl, C₃₋₆ cycloalkyl, halogen, —CN, and —OR^{100a}; wherein the C₁₋₆ alkyl and C₃₋₆ cycloalkyl are each optionally substituted with one to three substituents independently selected from halogen and C₁₋₃ alkyl; R^{100a}, for each occurrence, is independently selected from H, C₁₋₆ alkyl, C₃₋₆ cycloalkyl, and 4- to 6-membered monocyclic heterocyclyl; and the definitions for the other variables are as defined in the first, second, third, fourth, fifth, sixth, seventh, eighth, ninth, tenth, eleventh, twelfth, thirteenth, fourteenth, fifteenth, sixteenth, nineteenth, or twentieth embodiment.

In a twenty-second embodiment, a compound of the present invention is represented by Formula (I), (IIA), or (IIB), or a pharmaceutically acceptable salt thereof, wherein R¹⁰⁰ is C₁₋₆ alkyl or C₃₋₆ cycloalkyl; and the definitions for the other variables are as defined in the first, second, third, fourth, fifth, sixth, seventh, eighth, ninth, tenth, eleventh, twelfth, thirteenth, fourteenth, fifteenth, sixteenth, seventeenth, or twenty-first embodiment.

In a twenty-third embodiment, a compound of the present invention is represented by Formula (I), (IIA), or (IIB), or a pharmaceutically acceptable salt thereof, wherein R¹⁰⁰, for each occurrence, is independently —C(CH₃)₃, —CH₂C(CH₃)₃ or cyclopropyl; and the definitions for the other variables are as defined in the first, second, third, fourth, fifth, sixth, seventh, eighth, ninth, tenth, eleventh, twelfth, thirteenth, fourteenth, fifteenth, sixteenth, seventeenth, eighteenth, or twenty-second embodiment.

In a twenty-fourth embodiment, a compound of the present invention is represented by Formula (I), (IIA), or (IIB), or a pharmaceutically acceptable salt thereof, wherein R³ is H; and the definitions for the other variables are as defined in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, or twenty-third embodiment.

In a twenty-fifth embodiment, a compound of the present invention is represented by Formula (I), (IIA), or (IIB), or a pharmaceutically acceptable salt thereof, wherein R⁴ is selected from H, halogen, —CN, —OR^{4a}, C₁₋₆ alkyl, and C₃₋₆ cycloalkyl, wherein the C₁₋₆ alkyl and C₃₋₆ cycloalkyl represented by R⁴ are each optionally substituted with one to three halogen; and R^{4a} is C₁₋₄ alkyl optionally substituted with one to three halogen; and the definitions for the other variables are as defined in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, or twenty-first, twenty-second, twenty-third, or twenty-fourth embodiment.

In a twenty-sixth embodiment, a compound of the present invention is represented by Formula (I), (IIA), or (IIB), or a pharmaceutically acceptable salt thereof, wherein R⁴ is selected from H, halogen, —OR^{4a} and C₁₋₆ alkyl optionally substituted with one to three halogen; and R^{4a} is C₁₋₄ alkyl optionally substituted with one or three halogen; and the definitions for the other variables are as defined in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, or twenty-fifth embodiment.

In a twenty-seventh embodiment, a compound of the present invention is represented by Formula (I), (IIA), or (IIB), or a pharmaceutically acceptable salt thereof, wherein R⁴ is —CH₃; and the definitions for the other variables are as defined in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, or twenty-sixth embodiment.

In a twenty-eighth embodiment, a compound of the present invention is represented by Formula (I), (IIA), or (IIB), or a pharmaceutically acceptable salt thereof, wherein R³ and R⁴, together with their intervening atoms, form a Ring C, wherein Ring C is selected from 5- to 7-membered monocyclic carbocycle and 5- to 7-membered monocyclic heterocycle, wherein Ring C is optionally substituted with R³⁰⁰; and the definitions for the other variables are as defined in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, or twenty-third embodiment.

In a twenty-ninth embodiment, a compound of the present invention is represented by Formula (I), (IIA), or (IIB), or a pharmaceutically acceptable salt thereof, wherein R⁶ is H or halogen; and the definitions for the other variables are as defined in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, or twenty-eighth embodiment.

In a thirtieth embodiment, a compound of the present invention is represented by Formula (I), (IIA), or (IIB), or a pharmaceutically acceptable salt thereof, wherein R⁶ is H or F; and the definitions for the other variables are as defined in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, or twenty-ninth embodiment.

In a thirty-first embodiment, a compound of the present invention is represented by Formula (I), (IIA), or (IIB), or a pharmaceutically acceptable salt thereof, wherein R^{6A} is H, halogen or CN; and the definitions for the other variables are as defined in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, or thirtieth embodiment.

In a thirty-second embodiment, a compound of the present invention is represented by Formula (I), (IIA), or (IIB), or a pharmaceutically acceptable salt thereof, wherein R⁵ is H or —NHR^{5a}; and R^{5a} is H or C₁₋₃ alkyl; and the definitions for the other variables are as defined in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, or thirty-first embodiment.

In a thirty-third embodiment, a compound of the present invention is represented by Formula (I), (IIA), or (IIB), or a pharmaceutically acceptable salt thereof, wherein R⁵ is H; and the definitions for the other variables are as defined in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, or thirty-second embodiment.

In a thirty-fourth embodiment, a compound of the present invention is represented by Formula (I), (IIA), or (IIB), or a pharmaceutically acceptable salt thereof, wherein ring A is a 4 to 8-membered monocyclic saturated azacyclic ring, optionally substituted with one or two R¹¹; and the definitions for the other variables are as defined in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, thirty-second, or thirty-third embodiment.

In a thirty-fifth embodiment, a compound of the present invention is represented by Formula (I), (IIA), or (IIB), or a pharmaceutically acceptable salt thereof, wherein m is 1; and R⁹ and one R¹¹ together with intervening atoms form a 4- to 8-membered monocyclic saturated azacyclic ring; and the definitions for the other variables are as defined in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, thirty-second, thirty-third, or thirty-fourth embodiment.

In a thirty-sixth embodiment, a compound of the present invention is represented by Formula (I), (IIA), or (IIB), or a pharmaceutically acceptable salt thereof, wherein the azaleic ring is azetidine, pyrrolidine, piperidine, piperazine, azepane, or oxazepane; and the definitions for the other variables are as defined in the thirty-fourth or thirty-fifth embodiment.

In a thirty-seventh embodiment, a compound of the present invention is represented by Formula (I), (IIA), or (IIB), or a pharmaceutically acceptable salt thereof, wherein m is 0 and n is 0; and the definitions for the other variables are as defined in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-second, thirty-third, thirty-fourth, or thirty-sixth embodiment.

In a thirty-eighth embodiment, a compound of the present invention is represented by the following formula:

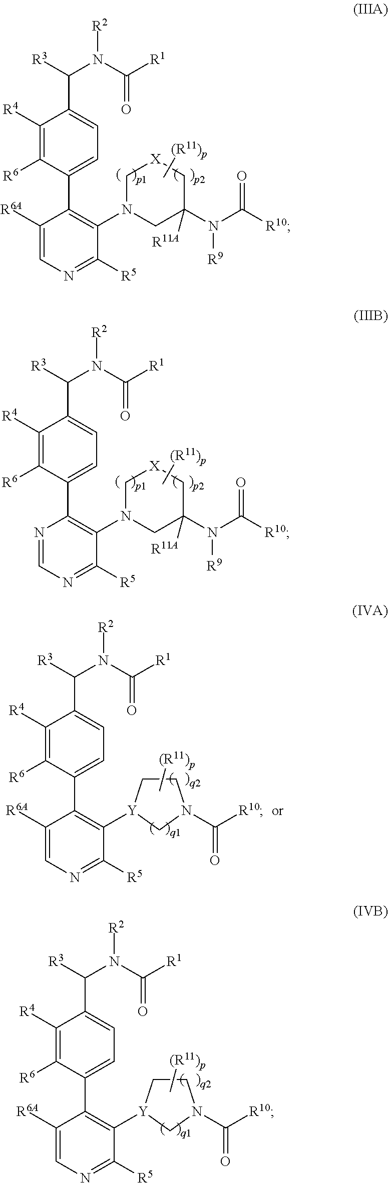

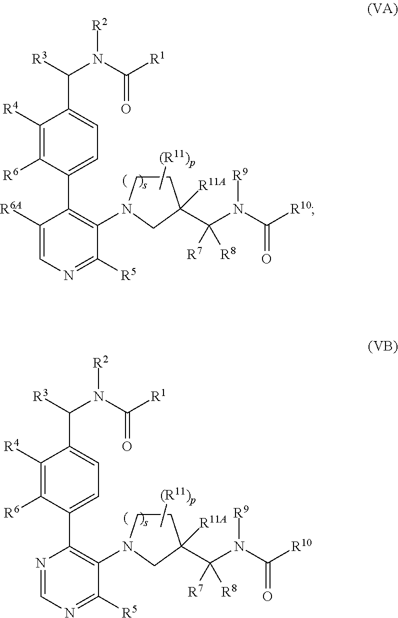

or a pharmaceutically acceptable salt thereof, wherein:
X is O or CHR^{11B};
R^{11A} is H; or R^{11A} and R⁹ together with their intervening atoms form a 4- to 6-membered saturated monocyclic azacyclic ring; R^{11B} is H; or R^{11B} and R⁹ together with their intervening atoms form a 4- to 6-membered saturated monocyclic azacyclic ring;
Y is CH or N;
p is 0, 1, 2, 3 or 4;
p1 is 0, 1, or 2;
p2 is 0, 1 or 2;
q1 is 0, 1, or 2, provided when Y is N, q1 is not 0;
q2 is 0, 1 or 2; provided that q1 and q2 cannot both be 0; and
s is 0, 1, or 2; and the definitions for the other variables are as defined in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, thirty-second, thirty-third, thirty-fourth, thirty-fifth, thirty-sixth, or thirty-seventh embodiment.

In a thirty-ninth embodiment, a compound of the present invention is represented by Formula (IIIA), (IIIB), (IVA), (IVB), (VA), or (VB), or a pharmaceutically acceptable salt thereof, wherein p1 is 1 or 2 and p2 is 0 or 1; q1 is 1 or 2 and q2 is 1 or 2; and s is 1 or 2; and the definitions for the other variables are as defined in thirty-eighth embodiment.

In a fortieth embodiment, a compound of the present invention is represented by the following formula:

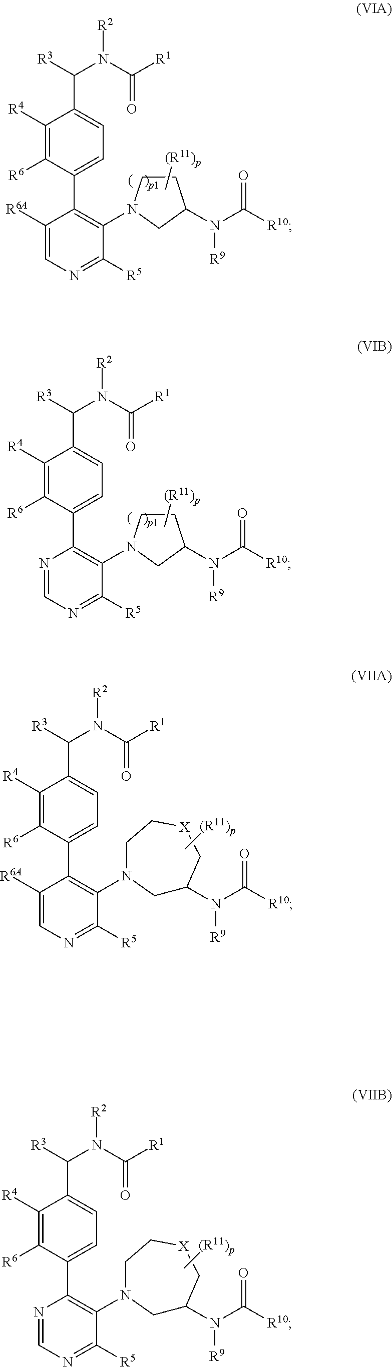

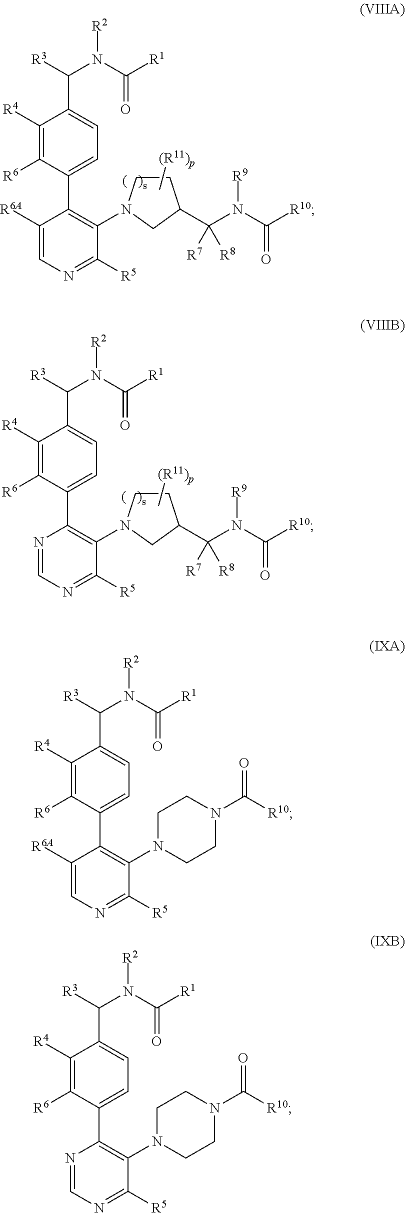

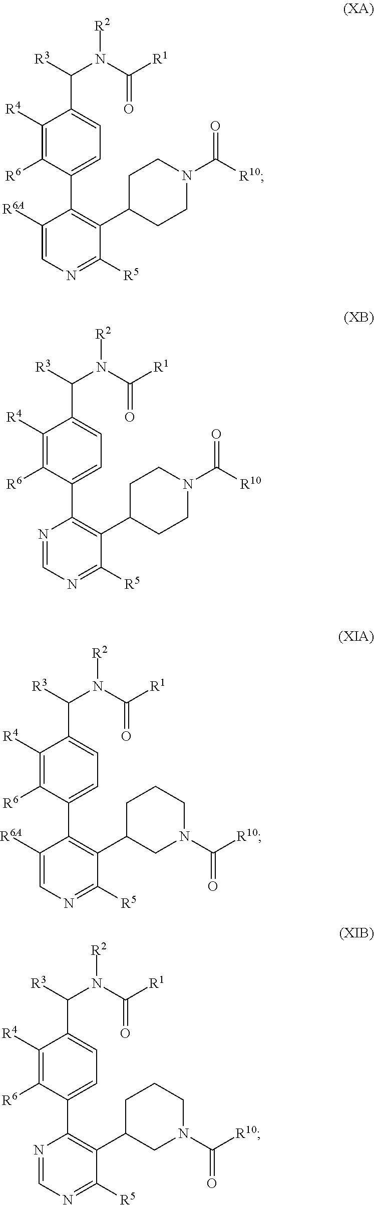

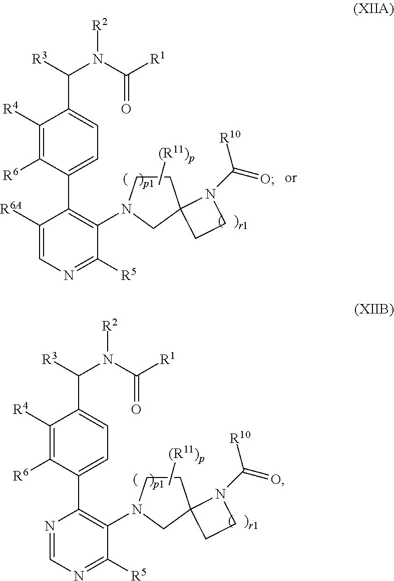

or a pharmaceutically acceptable salt thereof, wherein:
p1 is 1 or 2;
s is 1 or 2;
r1 is 1 or 2; and
X is O or CH₂; and the definitions for the other variables are as defined in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, thirty-second, thirty-third, thirty-fourth, thirty-fifth, thirty-sixth, thirty-seventh, or thirty-eighth embodiment.

In a forty-first embodiment, a compound of the present invention is represented by Formula (I), (IIA), (IIB), (IIIA), (IIIB), (IVA), (IVB), (VA), (VB), (VIA), (VIB), (VIIA), (VIIB), (VIIIA), (VIIIB), (IXA), (IXB), (XA), (XB), (XIA), (XIB), (XIIA), or (XIIB), or a pharmaceutically acceptable salt thereof, wherein p is 0, 1 or 2; and the definitions for the other variables are as defined in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, thirty-second, thirty-third, thirty-fourth, thirty-fifth, thirty-sixth, thirty-seventh, thirty-eighth, thirty-ninth, or fortieth embodiment.

In a forty-second embodiment, a compound of the present invention is represented by Formula (I), (IIA), (IIB), (IIIA), (IIIB), (IVA), (IVB), (VA), (VB), (VIA), (VIB), (VIIA), (VIIB), (VIIIA), (VIIIB), (IXA), (IXB), (XA), (XB), (XIA), (XIB), (XIIA), or (XIIB), or a pharmaceutically acceptable salt thereof, wherein p is 0; and the definitions for the other variables are as defined in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, thirty-second, thirty-third, thirty-fourth, thirty-fifth, thirty-sixth, thirty-seventh, thirty-eighth, thirty-ninth, fortieth, or forty-first embodiment.

In a forty-third embodiment, a compound of the present invention is represented by Formula (I), (IIA), (IIB), (IIIA), (IIIB), (IVA), (IVB), (VA), (VB), (VIA), (VIB), (VIIA), (VIIB), (VIIIA), (VIIIB), (IXA), (IXB), (XA), (XB), (XIA), (XIB), (XIIA), or (XIIB), or a pharmaceutically acceptable salt thereof, wherein p is 2 and two R¹¹ together with the same carbon atom from which they are attached form a —C(═O)— group; and the definitions for the other variables are as defined in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, thirty-second, thirty-third, thirty-fourth, thirty-fifth, thirty-sixth, thirty-seventh, thirty-eighth, thirty-ninth, fortieth, or forty-first embodiment.

In a forty-fourth embodiment, a compound of the present invention is represented by Formula (I), (IIA), (IIB), (IIIA), (IIIB), (IVA), (IVB), (VA), (VB), (VIA), (VIB), (VIIA), (VIIB), (VIIIA), (VIIIB), (IXA), (IXB), (XA), (XB), (XIA), (XIB), (XIIA), or (XIIB), or a pharmaceutically acceptable salt thereof, wherein R⁹ is C₁₋₃alkyl or C₃₋₆cycloalkyl; and the definitions for the other variables are as defined in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, thirty-second, thirty-third, thirty-fourth, thirty-fifth, thirty-sixth, thirty-seventh, thirty-eighth, thirty-ninth, fortieth, forty-first, forty-second, or forty-third embodiment.

In a forty-fifth embodiment, a compound of the present invention is represented by Formula (I), (IIA), (IIB), (IIIA), (IIIB), (IVA), (IVB), (VA), (VB), (VIA), (VIB), (VIIA), (VIIB), (VIIIA), (VIIIB), (IXA), (IXB), (XA), (XB), (XIA), (XIB), (XIIA), or (XIIB), or a pharmaceutically acceptable salt thereof, wherein R⁹ is —CH₃, —CH₂CH₃ or cyclopropyl; and the definitions for the other variables are as defined in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, thirty-second, thirty-third, thirty-fourth, thirty-fifth, thirty-sixth, thirty-seventh, thirty-eighth, thirty-ninth, fortieth, forty-first, forty-second, forty-third, or forty-fourth embodiment.

In a forty-sixth embodiment, a compound of the present invention is represented by Formula (I), (IIA), (IIB), (IIIA), (IIIB), (IVA), (IVB), (VA), (VB), (VIA), (VIB), (VIIA), (VIIB), (VIIIA), (VIIIB), (IXA), (IXB), (XA), (XB), (XIA), (XIB), (XIIA), or (XIIB), or a pharmaceutically acceptable salt thereof, wherein R¹⁰ is C₂₋₆ alkenyl optionally substituted with C₁₋₆ alkyl, C₁₋₆ alkoxy or —NR^{10a}R^{10b}, and R^{10a} and R^{10b} are each independently H or C₁₋₃ alkyl; and the definitions for the other variables are as defined in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, thirty-second, thirty-third, thirty-fourth, thirty-fifth, thirty-sixth, thirty-seventh, thirty-eighth, thirty-ninth, fortieth, forty-first, forty-second, forty-third, forty-fourth, or forty-fifth embodiment.

In a forty-seventh embodiment, a compound of the present invention is represented by Formula (I), (IIA), (IIB), (IIIA), (IIIB), (IVA), (IVB), (VA), (VB), (VIA), (VIB), (VIIA), (VIIB), (VIIIA), (VIIIB), (IXA), (IXB), (XA), (XB), (XIA), (XIB), (XIIA), or (XIIB), or a pharmaceutically acceptable salt thereof, wherein R¹⁰ is-CH═CH₂; and the definitions for the other variables are as defined in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, thirty-second, thirty-third, thirty-fourth, thirty-fifth, thirty-sixth, thirty-seventh, thirty-eighth, thirty-ninth, fortieth, forty-first, forty-second, forty-third, forty-fourth, forty-fifth, or forty-sixth embodiment.

In a forty-eighth embodiment, a compound of the present invention is represented by Formula (I), (IIA), (IIB), (IIIA), (IIIB), (IVA), (IVB), (VA), (VB), (VIA), (VIB), (VIIA), (VIIB), (VIIIA), (VIIIB), (IXA), (IXB), (XA), (XB), (XIA), (XIB), (XIIA), or (XIIB), or a pharmaceutically acceptable salt thereof, wherein: wherein R¹⁰ is C₂₋₆ alkenyl or C₄₋₇ cycloalkenyl optionally substituted with one or more halo, C₁₋₆ alkyl, C₁₋₆ alkoxy or —NR^{10a}R^{10b}, and R^{10a} and R^{10b} are each independently H or C₁₋₃ alkyl, or R^{10a} and R^{10b} together with the nitrogen atom from they are they are attached form a 4- to 7-membered monocyclic saturated heterocyclyl optionally substituted with one or more substituents independently selected from halo and C₁₋₆ alkyl; and the definitions for the other variables are as defined in the first, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, thirty-second, thirty-third, thirty-fourth, thirty-fifth, thirty-sixth, thirty-seventh, thirty-eighth, thirty-ninth, fortieth, forty-first, forty-second, forty-third, forty-fourth, or forty-fifth embodiment.

In a forty-ninth embodiment, a compound of the present invention is represented by Formula (I), (IIA), (IIB), (IIIA), (IIIB), (IVA), (IVB), (VA), (VB), (VIA), (VIB), (VIIA), (VIIB), (VIIIA), (VIIIB), (IXA), (IXB), (XA), (XB), (XIA), (XIB), (XIIA), or (XIIB), or a pharmaceutically acceptable salt thereof, wherein R¹⁰ is —CH═CH₂, CF═CH₂, —CH═CHCF₃,

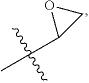 [SMILES: *C1CO1]

—CCH, —CCCH₃, -cyclobutene, azetidino, morpholino or piperazino, optionally substituted with halo or methyl and the definitions for the other variables are as defined in the forty-eighth embodiment.

In a fiftieth embodiment, a compound of the present invention is represented by Formula (I), (IIA), (IIB), (IIIA), (IIIB), (IVA), (IVB), (VA), (VB), (VIA), (VIB), (VIIA), (VIIB), (VIIIA), (VIIIB), (IXA), (IXB), (XA), (XB), (XIA), (XIB), (XIIA), or (XIIB), or a pharmaceutically acceptable salt thereof, wherein R¹ is represented by the following formula:

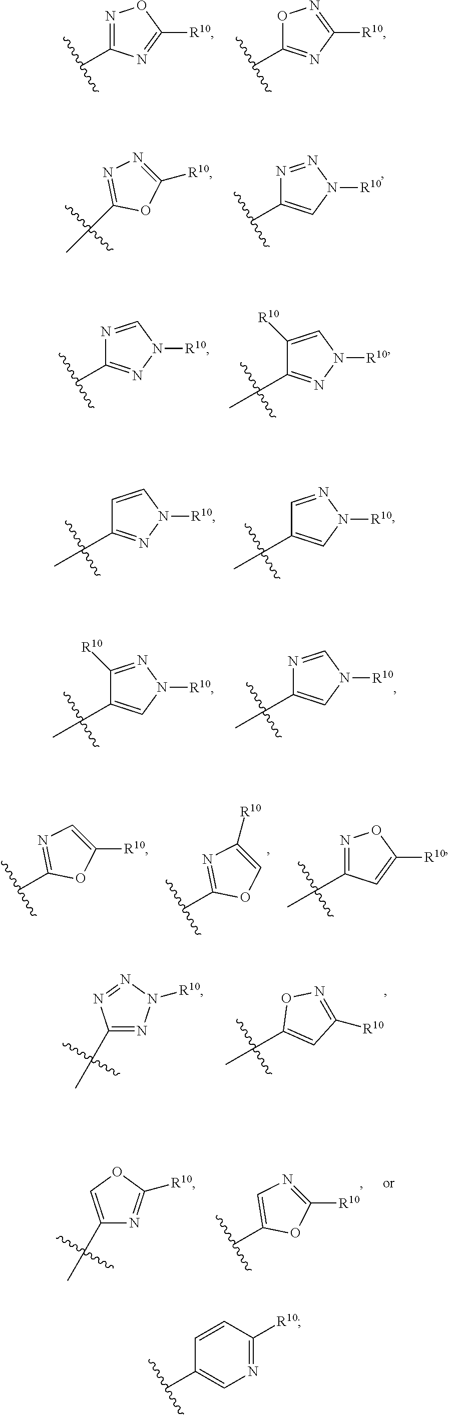

R¹⁰, for each occurrence, is independently C₁₋₄ alkyl optionally substituted with one to three halogen or a C₃₋₆cycloalkyl optionally substituted with one or two C₁₋₃alkyl;
R² is H or C₁₋₃alkyl;
R³ is H;
R⁴ is C₁₋₃alkyl;
R⁵ is H;
R⁶ is H or halogen; and start here
R^{6A} is H, halogen or CN; and the definitions for the other variables are as defined in the thirty-eighth, thirty-ninth, fortieth, forty-first, forty-second, forty-third, forty-fourth, forty-fifth, forty-sixth, or forty-seventh embodiment.

In a fifty-first embodiment, a compound of the present invention is represented by Formula (I), (IIA), (IIB), (IIIA), (IIIB), (IVA), (IVB), (VA), (VB), (VIA), (VIB), (VIIA), (VIIB), (VIIIA), (VIIIB), (IXA), (IXB), (XA), (XB), (XIA), (XIB), (XIIA), or (XIIB), or a pharmaceutically acceptable salt thereof, wherein:
R¹ is represented by the following formula:

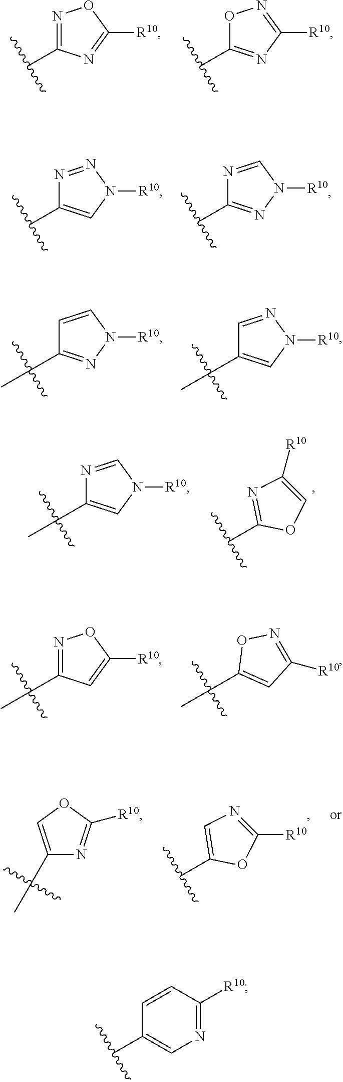

R¹⁰, for each occurrence, is independently C₁₋₄ alkyl optionally substituted with one to three halogen or a cyclopropyl optionally substituted with one C₁₋₃alkyl;
R² is H;
R³ is H;
R⁴ is —CH₃;
R⁵ is H;
R⁶ is H or F; and
R^{6A} is H or CN; and the definitions for the other variables are as defined in the thirty-eighth, thirty-ninth, fortieth, forty-first, forty-second, forty-third, forty-fourth, forty-fifth, forty-sixth, forty-seventh, forty-eighth, or forty-ninth embodiment.

As used herein, the term “alkyl” refers to a fully saturated branched or unbranched hydrocarbon moiety. Preferably the alkyl comprises 1 to 6 carbon atoms, or 1 to 4 carbon atoms.

In some embodiments, an alkyl comprises from 6 to 20 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, or n-hexyl.

“Alkenyl” refers to an unsaturated hydrocarbon group which may be linear or branched and has at least one carbon-carbon double bond. Alkenyl groups with 2-6 carbon atoms can be preferred. The alkenyl group may contain 1 or more. Examples of alkenyl groups include ethenyl, n-propenyl, iso-propenyl, n-but-2-enyl, n-hex-3-enyl and the like.

“Alkynyl” refers to an unsaturated hydrocarbon group which may be linear or branched and has at least one carbon-carbon triple bond. Alkynyl groups with 2-6 carbon atoms can be preferred. The alkynyl group may contain 1 or more. Examples of alkynyl groups include ethynyl, n-propynyl, n-but-2-ynyl, n-hex-3-ynyl and the like.

The number of carbon atoms in a group is specified herein by the prefix “Cₓ₋ₓₓ”, wherein x and xx are integers. For example, “C₁₋₄ alkyl” is an alkyl group which has from 1 to 6 carbon atoms.

“Halogen” or “halo” may be fluoro, chloro, bromo or iodo.

As used herein, the term “heterocyclyl” refers to a saturated or unsaturated, monocyclic or bicyclic (e.g., fused, bridged or spiro ring systems) ring system which has from 3- to 10-ring members, or in particular 3- to 8-ring members, 3- to 7-ring members, 3- to 6-ring members, 5 to 7-ring members, 4- to 8-ring members, 4- to 7-ring members, 4- to 6-ring members or 7- to 10-ring members, at least one of which is a heteroatom, and up to 4 (e.g., 1, 2, 3, or 4) of which may be heteroatoms, wherein the heteroatoms are independently selected from O, S and N, and wherein C can be optionally oxidized (e.g., C(O)), N can be optionally oxidized (e.g., N(O)) or quaternized, and S can be optionally oxidized to sulfoxide and sulfone. Unsaturated heterocyclic rings include heteroaryl rings. The heterocyclyl group can be attached to the rest of a compound of the invention at a heteroatom or a carbon atom. The term azacyclic refers to a non-aromatic heterocyclyl which has at least one nitrogen ring atom. The examples of azacyclic include, but are not limited to, morpholine.

In one embodiment, a heterocyclyl is a 3- to 7-membered monocyclic heterocyclyl (saturated or partially unsaturated (i.e., non-aromatic)) having 1-2 heteroatoms selected from O, S and N. Examples of 3- to 7-membered monocyclic heterocyclyl include, but are not limited to, aziridinyl, oxiranyl, thirranyl, oxaziridinyl, azetidinyl, oxetanyl, thietanyl, pyrrolidinyl, tetrahydrofuranyl, thiolanyl, imidazolidinyl, pyrazolidinyl, oxazolidinyl, isoxazolidinyl, thiazolidinyl, isothiazolidinyl, dioxolanyl, dithiolanyl, oxathiolanyl, piperidinyl, tetrahydropyranyl, thianyl, piperazinyl, morpholinyl, thiomorpholinyl, dioxanyl, dithianyl, trioxanyl, trithianyl, azepanyl, oxepanyl, thiepanyl, dihydrofuranyl, imidazolinyl, and dihydropyranyl. In one embodiment, a heterocyclyl is a 5-to 7-membered monocyclic heterocyclyl (saturated or partially unsaturated). Examples include pyrrolidinyl, tetrahydrofuranyl, thiolanyl, imidazolidinyl, pyrazolidinyl, oxazolidinyl, isoxazolidinyl, thiazolidinyl, isothiazolidinyl, dioxolanyl, dithiolanyl, oxathiolanyl, piperidinyl, tetrahydropyranyl, thianyl, piperazinyl, morpholinyl, thiomorpholinyl, dioxanyl, dithianyl, trioxanyl, trithianyl, azepanyl, oxepanyl, thiepanyl, dihydrofuranyl, imidazolinyl, and dihydropyranyl.

In another embodiment, a heterocyclyl is a 4- to 6-membered monocyclic heterocyclyl (saturated or partially unsaturated) having 1-2 heteroatoms selected from O, S and N. Examples of a 4- to 6-membered monocyclic heterocyclic include, but are not limited to azetidinyl, oxetanyl, thietanyl, pyrrolidinyl, tetrahydrofuranyl, thiolanyl, imidazolidinyl, pyrazolidinyl, oxazolidinyl, isoxazolidinyl, thiazolidinyl, isothiazolidinyl, dioxolanyl, dithiolanyl, oxathiolanyl, piperidinyl, tetrahydropyranyl, thianyl, piperazinyl, morpholinyl, thiomorpholinyl, dioxanyl, dithianyl, trioxanyl, trithianyl, dihydrofuranyl, imidazolinyl, and dihydropyranyl.

In one embodiment, a heterocyclyl is a 4- to 6-membered monocyclic heterocyclyl (unsaturated, partially unsaturated or saturated) having 1-2 heteroatoms selected from O, S and N. Examples of a 4- to 6-membered monocyclic heterocyclic include, but are not limited to azetidinyl, pyrrolidinyl, tetrahydrofuranyl, thiolanyl, imidazolidinyl, pyrazolidinyl, oxazolidinyl, isoxazolidinyl, thiazolidinyl, isothiazolidinyl, dioxolanyl, dithiolanyl, oxathiolanyl, piperidinyl, tetrahydropyranyl, thianyl, piperazinyl, morpholinyl, thiomorpholinyl, dioxanyl, dithianyl, dihydrofuranyl, imidazolinyl, dihydropyranyl, pyrrolyl, furanyl, thiophenyl (or thienyl), imidazolyl, pyrazolyl, oxazolyl, isoxazolyl, thiazolyl, isothiazolyl, furazanyl, oxadiazolyl, thiadiazolyl, dithiazolyl, triazolyl, tetrazolyl, pyridinyl, pyranyl, thiopyranyl, pyrazinyl, pyrimidinyl, pyridazinyl, oxazinyl, thiazinyl, dioxinyl, dithiinyl, oxathianyl, triazinyl, and tetrazinyl.

In another embodiment, a heterocyclyl is a 5- to 7-membered monocyclic heterocyclyl (unsaturated, partially unsaturated or saturated) having 1-2 heteroatoms selected from O, S and N. Examples of a 4- to 6-membered monocyclic heterocyclic include, but are not limited to pyrrolidinyl, tetrahydrofuranyl, thiolanyl, imidazolidinyl, pyrazolidinyl, oxazolidinyl, isoxazolidinyl, thiazolidinyl, isothiazolidinyl, dioxolanyl, dithiolanyl, oxathiolanyl, piperidinyl, tetrahydropyranyl, thianyl, piperazinyl, morpholinyl, thiomorpholinyl, dioxanyl, dithianyl, dihydrofuranyl, imidazolinyl, dihydropyranyl, pyrrolyl, furanyl, thiophenyl (or thienyl), imidazolyl, pyrazolyl, oxazolyl, isoxazolyl, thiazolyl, isothiazolyl, furazanyl, oxadiazolyl, thiadiazolyl, dithiazolyl, triazolyl, tetrazolyl, pyridinyl, pyranyl, thiopyranyl, pyrazinyl, pyrimidinyl, pyridazinyl, oxazinyl, thiazinyl, dioxinyl, dithiinyl, oxathianyl, triazinyl, and tetrazinyl. azepanyl, oxepanyl and thiepanyl.

In another embodiment, a heterocyclyl is a 4- to 7-membered monocyclic heterocyclyl (saturated or partially unsaturated) having 1-2 heteroatoms selected from O, S and N. Examples of a 4- to 7-membered monocyclic heterocyclic include, but are not limited to azetidinyl, oxetanyl, thietanyl, pyrrolidinyl, tetrahydrofuranyl, thiolanyl, imidazolidinyl, pyrazolidinyl, oxazolidinyl, isoxazolidinyl, thiazolidinyl, isothiazolidinyl, dioxolanyl, dithiolanyl, oxathiolanyl, piperidinyl, tetrahydropyranyl, thianyl, piperazinyl, morpholinyl, thiomorpholinyl, dioxanyl, dithianyl, trioxanyl, trithianyl, azepanyl, oxepanyl, thiepanyl, dihydrofuranyl, imidazolinyl, and dihydropyranyl.

In another embodiment, a heterocyclyl is a saturated 4- to 6-membered monocyclic heterocyclyl having 1-2 heteroatoms selected from O, S and N. Examples of saturated 4- to 6-membered monocyclic heterocyclic ring systems include, but are not limited to azetidinyl, pyrrolidinyl, tetrahydrofuranyl, thiolanyl, imidazolidinyl, pyrazolidinyl, oxazolidinyl, isoxazolidinyl, thiazolidinyl, isothiazolidinyl, dioxolanyl, dithiolanyl, oxathiolanyl, piperidinyl, tetrahydropyranyl, thianyl, piperazinyl, morpholinyl, thiomorpholinyl, dioxanyl, and dithiinyl.

In one embodiment, a saturated 4- to 6-membered monocyclic heterocyclyl is azetidinyl, oxetanyl, pyrrolidinyl, tetrahydrofuranyl, thiolanyl, imidazolidinyl, pyrazolidinyl, oxazolidinyl, isoxazolidinyl, thiazolidinyl, isothiazolidinyl, dioxolanyl, dithiolanyl, oxathiolanyl, piperidinyl, tetrahydropyranyl, thianyl, piperazinyl, morpholinyl, thiomorpholinyl, or dioxinyl. In another embodiment, a saturated 4- to 6-membered monocyclic heterocyclyl is oxetanyl, tetrahydrofuranyl, or tetrahydropyranyl.

In one embodiment, a 4- to 6-membered monocyclic heterocyclyl is selected from

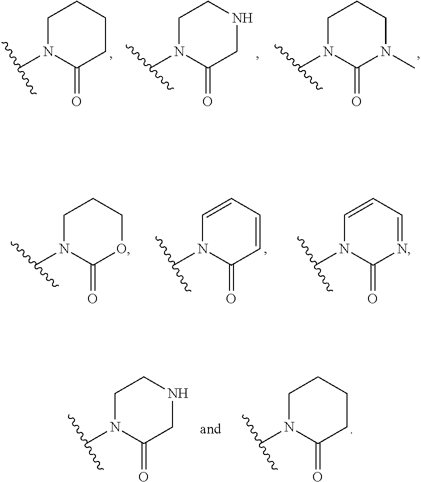

In another embodiment, a 4- to 6-membered monocyclic heterocyclyl is selected from

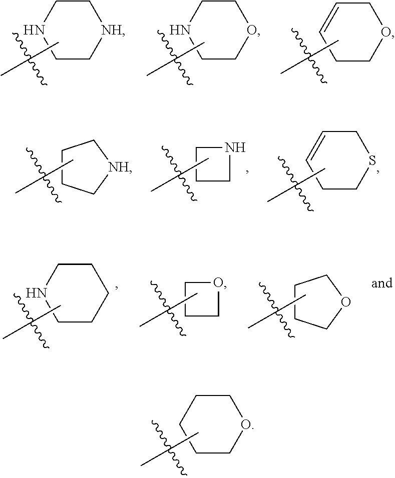

In one embodiment, a heterocyclyl is a 7-membered monocyclic heterocyclyl (saturated or partially unsaturated), such as a 7-membered monocyclic heterocyclyl having one heteroatom selected from O and N. Examples of a 7-membered monocyclic heterocyclyl include, but are not limited to, azepanyl, azepinyl, oxepanyl, oxepinyl, thiepanyl, thiepinyl, diazepanyl, diazepinyl, and thiazepinyl.

In another embodiment, a heterocyclyl is a 7- to 10-membered bicyclic heterocyclyl. In yet another embodiment, a heterocyclyl is a 9- to 10-membered non-aromatic bicyclic heterocyclyl. In another embodiment, a heterocyclyl is 9- to 10-membered fused non-aromatic bicyclic heterocyclyl. The heterocyclyl group can be attached to the rest of a compound of the invention at a heteroatom or a carbon atom. In one embodiment, a 9- to 10-membered fused non-aromatic bicyclic heterocyclyl is selected from

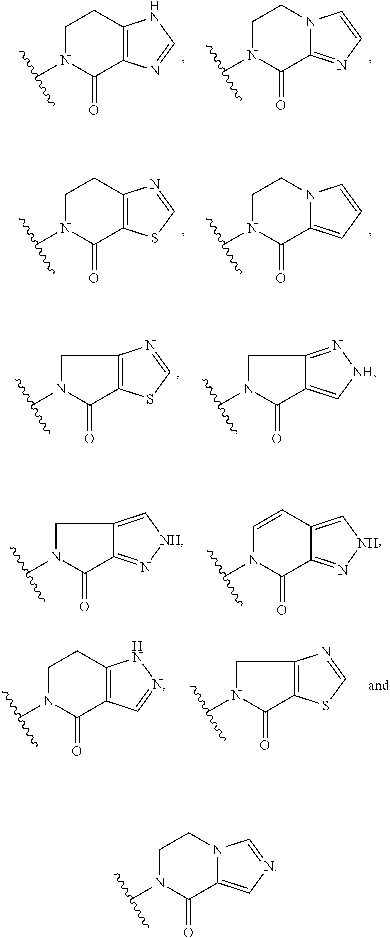

In another embodiment, a heterocyclyl is a 7- to 8-membered bridged non-aromatic bicyclic heterocyclyl, such as

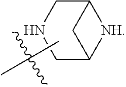

In another embodiment, a heterocyclyl is a 4-8 membered monocyclic saturated azacyclic ring. Examples include azetidinyl, pyrrolidinyl, imidazolidinyl, pyrazolidinyl, oxazolidinyl, isoxazolidinyl, thiazolidinyl, isothiazolidinyl, piperidinyl, piperazinyl, morpholinyl, thiomorpholinyl, azepanyl, oxazepanyl, and imidazolinyl. In another embodiment, a heterocyclyl is a 4-6 membered monocyclic saturated azacyclic ring. Examples include azetidinyl, pyrrolidinyl, imidazolidinyl, pyrazolidinyl, oxazolidinyl, isoxazolidinyl, thiazolidinyl, isothiazolidinyl, piperidinyl, piperazinyl, morpholinyl, thiomorpholinyl, oxazepanyl and imidazolinyl.

As used herein, the term “aryl” refers to a carbocyclic (all carbon) aromatic monocyclic or bicyclic ring system containing 6-10 or 9-10 carbon atoms. Examples of 6-10 membered aryl groups include phenyl, naphthyl and tetrahydronaphthyl. Examples of 9-10 membered bicyclic aryl groups include naphthyl.

As used herein, the term “heteroaryl” refers to an aromatic 5- to 6-membered monocyclic or a 7- to 10-membered bicyclic ring system, having 1 to 4 heteroatoms independently selected from O, N and S, and wherein N can be oxidized (e.g., N(O)) or quaternized, and S can be optionally oxidized to sulfoxide and sulfone. Examples of 5- to 6-membered monocyclic heteroaryls include, but are not limited to, pyrrolyl, furanyl, thiophenyl (or thienyl), imidazolyl, pyrazolyl, oxazolyl, isoxazolyl, thiazolyl, isothiazolyl, furazanyl, oxadiazolyl, thiadiazolyl, dithiazolyl, triazolyl, tetrazolyl, pyridinyl, pyrazinyl, pyrimidinyl, pyridazinyl, triazinyl, tetrazinyl, and the like. In one embodiment, a heteroaryl is a 5-membered heteroaryl. Examples of a 5-membered heteroaryl include, but are not limited to, pyrazolyl, imidazolyl, oxazolyl, isoxazolyl, 1,2,3-oxadiazolyl, 1,3,4-oxadiazolyl, 1,2,4-oxadizolyl, 1,2,3-thiadiazolyl, 1,3,4-thiadiazolyl, 1,2,4-thiadiazolyl, 1,2,3-triazolyl, 1,2,4-triazolyl, and tetrazolyl. Examples of 8- to 10-membered bicyclic heteroaryls include, but are not limited to, dihydropyrrolopyrrolyl, indolyl, isoindolyl, benzimidazolyl, benzothiazolyl, quinolinyl, isoquinolinyl, quinazolinyl and purinyl.

As used herein, the term “carbocyclyl” refers to saturated or unsaturated monocyclic or bicyclic hydrocarbon groups of 3-10, 3-8, 3-7, 3-5, 3-6, 4-6, 5-7 or 7-10 carbon atoms. The term “carbocyclyl” encompasses cycloalkyl groups and aromatic groups (i.e., aryl). The term “cycloalkyl” refers to completely saturated monocyclic or bicyclic or spiro hydrocarbon groups of 3-7 carbon atoms, 3-6 carbon atoms, or 5-7 carbon atoms. Exemplary bicyclic carbocyclyl groups include bicyclo[2.1.1]hexyl, bicyclo[2.2.1]heptyl, bicyclo[2.2.1]heptenyl, 6,6-dimethylbicyclo[3.1.1]heptyl, 2,6,6-trimethylbicyclo[3.1.1]heptyl, spiro[2.2]pentanyl, and spiro[3.3]heptanyl.

As used herein, the term “cycloalkenyl” refers to a non-aromatic monocyclic hydrocarbon ring system with four to seven ring atoms and at least one double bond between two adjacent carbon atoms in the ring system. For example, “C₄₋₇ cycloalkenyl” can refer to cyclobutene, cyclopentene, cyclohexene, or cycloheptene. In some cases, one double bond may be present in the ring structure. In other cases, more than one double bond may be present (e.g.,

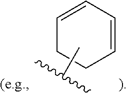

As used here, the term “alkylene oxide” refers an alkyl group in which a hydrogen atom on each of two adjacent carbon atoms is replaced with an oxygen atom bridging the two carbon atoms (e.g., an epoxide). One or more substitutions may be present along the alkyl group.

In one embodiment, the carbocyclyl is a 7- to 10-membered bicyclic carbocyclyl.

Exemplary 7- to 10-membered bicyclic carbocyclyls include, but are not limited to, bicyclo[2.2.1]heptyl, bicyclo[2.2.1]heptenyl, 6,6-dimethylbicyclo[3.1.1]heptyl, 2,6,6-trimethylbicyclo[3.1.1]heptyl, spiro[3.3]heptanyl, bicyclo[3.3.0]octanyl, bicyclo[2.2.2]octanyl, bicyclo[3.3.1]nonanyl, bicyclo[3.3.2]decanyl, decalinyl and indanyl. In one embodiment, the carbocyclyl is a 3- to 7-membered monocyclic carbocyclyl. Exemplary 3 to 7-membered monocyclic carbocyclyls include, but are not limited to, cyclopropyl, cyclobutyl, cyclopentyl, cyclohexyl, cycloheptyl, cyclopropenyl, cyclobutenyl, cyclopenentyl, cyclohexenyl, cycloheptenyl, cyclobutadienyl, cyclopentadienyl, cyclohexadienyl, cycloheptadienyl, phenyl and cycloheptatrienyl. In one embodiment, the carbocyclyl is a 5- to 7-membered monocyclic carbocyclyl, such as but not limited to cyclopentyl, cyclohexyl, cycloheptyl, cyclopenentyl, cyclohexenyl, cycloheptenyl, cyclopentadienyl, cyclohexadienyl, cycloheptadienyl, phenyl or cycloheptatrienyl. In another embodiment, the carbocyclyl is a 4- to 6-membered monocyclic carbocyclyl, such as but not limited to cyclobutyl, cyclopentyl, cyclohexyl, cyclobutenyl, cyclopenentyl, cyclohexenyl, cyclobutadienyl, cyclopentadienyl, cyclohexadienyl or phenyl. In another embodiment, the carbocyclyl is a 3- to 6-membered carbocycly, such as but not limited to cyclopropyl, cyclobutyl, cyclopentyl, cyclohexyl, cyclopropenyl, cyclobutenyl, cyclopenentyl, cyclohexenyl, cyclobutadienyl, cyclopentadienyl, cyclohexadienyl or phenyl. In another embodiment, the carbocyclyl is a 3- to 6-membered cycloalkyl, such as but not limited to cyclopropyl, cyclobutyl, cyclopentyl or cyclohexyl. In yet another embodiment, the carbocyclyl is phenyl. In yet another embodiment, the carbocyclyl is cyclopropyl.

The term “fused ring system”, as used herein, is a ring system that has two rings each of which are independently selected from a carbocyclyl or a heterocyclyl, wherein the two ring structures share two adjacent ring atoms. In one embodiment, a fused ring system have from 9 to 12 ring members.

The term “bridged ring system”, as used herein, is a ring system that has a carbocyclyl or heterocyclyl ring wherein two non-adjacent atoms of the ring are connected (bridged) by one or more (preferably from one to three) atoms selected from C, N, O, and S. In one embodiment, a bridged ring system have from 6 to 8 ring members.

The term “spiro ring system,” as used herein, is a ring system that has two rings each of which are independently selected from a carbocyclyl or a heterocyclyl, wherein the two ring structures having one ring atom in common. In one embodiment, spiro ring systems have from 5 to 8 ring members.

In cases where a compound provided herein is sufficiently basic or acidic to form stable nontoxic acid or base salts, preparation and administration of the compounds as pharmaceutically acceptable salts may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, a-ketoglutarate, or a-glycerophosphate. Inorganic salts may also be formed, including hydrochloride, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

Pharmaceutically-acceptable base addition salts can be prepared from inorganic and organic bases. Salts from inorganic bases, can include but are not limited to, sodium, potassium, lithium, ammonium, calcium or magnesium salts. Salts derived from organic bases can include, but are not limited to, salts of primary, secondary or tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amine, trisubstituted cycloalkyl amines, cycloalkenyl amines, di(cycloalkenyl) amines, tri(cycloalkenyl) amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocycloalkyl amines, diheterocycloalkyl amines, triheterocycloalkyl amines, or mixed di- and tri-amines where at least two of the substituents on the amine can be different and can be alkyl, substituted alkyl, alkenyl, substituted alkenyl, cycloalkyl, substituted cycloalkyl, cycloalkenyl, substituted cycloalkenyl, aryl, heteroaryl, or heterocycloalkyl and the like. Also included are amines where the two or three substituents, together with the amino nitrogen, form a heterocycloalkyl or heteroaryl group. Non-limiting examples of amines can include, isopropylamine, trimethyl amine, diethyl amine, tri(iso-propyl) amine, tri(n-propyl) amine, ethanolamine, 2-dimethylaminoethanol, trimethamine, lysine, arginine, histidine, caffeine, procaine, hydrabamine, choline, betaine, ethylenediamine, glucosamine, N-alkylglucamines, theobromine, purines, piperazine, piperidine, morpholine, or N-ethylpiperidine, and the like. Other carboxylic acid derivatives can be useful, for example, carboxylic acid amides, including carboxamides, lower alkyl carboxamides, or dialkyl carboxamides, and the like.

The compounds or pharmaceutically acceptable salts thereof as described herein, can contain one or more asymmetric centers in the molecule. In accordance with the present disclosure any structure that does not designate the stereochemistry is to be understood as embracing all the various stereoisomers (e.g., diastereomers and enantiomers) in pure or substantially pure form, as well as mixtures thereof (such as a racemic mixture, or an enantiomerically enriched mixture). It is well known in the art how to prepare such optically active forms (for example, resolution of the racemic form by recrystallization techniques, synthesis from optically-active starting materials, by chiral synthesis, or chromatographic separation using a chiral stationary phase).

When a particular stereoisomer of a compound is depicted by name or structure, the stereochemical purity of the compounds is at least 20%, 30%, 40%, 50%, 60%, 70%, 80%, 90%, 95%, 97%, 99%, 99.5% or 99.9%. “Stereochemical purity” means the weight percent of the desired stereoisomer relative to the combined weight of all stereoisomers.

When the stereochemistry of a disclosed compound is named or depicted by structure, and the named or depicted structure encompasses more than one stereoisomer (e.g., as in a diastereomeric pair), it is to be understood that one of the encompassed stereoisomers or any mixture of the encompassed stereoisomers are included. It is to be further understood that the stereoisomeric purity of the named or depicted stereoisomers at least 20%, 30%, 40%, 50%, 60%, 70%, 80%, 90%, 95%, 97%, 99%, 99.5% or 99.9%. The stereoisomeric purity the weight percent of the desired stereoisomers encompassed by the name or structure relative to the combined weight of all of the stereoisomers.

When a disclosed compound is named or depicted by structure without indicating the stereochemistry, and the compound has one chiral center, it is to be understood that the name or structure encompasses one enantiomer of compound in pure or substantially pure form, as well as mixtures thereof (such as a racemic mixture of the compound and mixtures enriched in one enantiomer relative to its corresponding optical isomer).

When a disclosed compound is named or depicted by structure without indicating the stereochemistry and, e.g., the compound has at least two chiral centers, it is to be understood that the name or structure encompasses one stereoisomer in pure or substantially pure form, as well as mixtures thereof (such as mixtures of stereoisomers, and mixtures of stereoisomers in which one or more stereoisomers is enriched relative to the other stereoisomer(s)).

The disclosed compounds may exist in tautomeric forms and mixtures and separate individual tautomers are contemplated.

In one embodiment, the invention provides deuterated compounds disclosed herein, in which any or more positions occupied by hydrogen can include enrichment by deuterium above the natural abundance of deuterium. For example, one or more hydrogen atoms are replaced with deuterium at an abundance that is at least 3340 times greater than the natural abundance of deuterium, which is 0.015% (i.e., at least 50.1% incorporation of deuterium), at least 3500 (52.5% deuterium incorporation at each designated deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). In one embodiment, hydrogen is present at all positions at its natural abundance.

The compounds or pharmaceutically acceptable salts thereof as described herein, may exist in tautomeric forms and mixtures and separate individual tautomers are contemplated.

Another embodiment is a pharmaceutical composition comprising at least one compound described herein, or a pharmaceutically acceptable salt thereof, and at least one pharmaceutically acceptable carrier.

The compounds, or pharmaceutically acceptable salts thereof described herein may be used to decrease the activity of Btk, or to otherwise affect the properties and/or behavior of Btk, e.g., stability, phosphorylation, kinase activity, interactions with other proteins, etc.

In some embodiments, the present invention provides methods of decreasing Btk enzymatic activity. In some embodiments, such methods include contacting a Btk with an effective amount of a Btk inhibitor. Therefore, the present invention further provides methods of inhibiting Btk enzymatic activity by contacting a Btk with a Btk inhibitor of the present invention.

One embodiment of the invention includes a method of treating a disorder responsive to inhibition of Btk in a subject comprising administering to the subject an effective amount of at least one compound described herein, or a pharmaceutically acceptable salt thereof, or a pharmaceutical composition described herein.

In one embodiment, the present invention provides methods of treating autoimmune disorders, inflammatory disorders, and cancers in a subject in need thereof comprising administering to the subject an effective amount of at least one compound described herein, or a pharmaceutically acceptable salt thereof, or a pharmaceutical composition described herein.

The term “autoimmune disorders” includes diseases or disorders involving inappropriate immune response against native antigens, such as acute disseminated encephalomyelitis (ADEM), Addison's disease, alopecia areata, antiphospholipid antibody syndrome (APS), autoimmune hemolytic anemia, autoimmune hepatitis, bullous pemphigoid (BP), Coeliac disease, dermatomyositis, diabetes mellitus type 1, Goodpasture's syndrome, Graves' disease, Guillain-Barre syndrome (GBS), Hashimoto's disease, idiopathic thrombocytopenic purpura, lupus erythematosus, mixed connective tissue disease, multiple sclerosis, myasthenia gravis, pemphigus vulgaris, pernicious anaemia, polymyositis, primary biliary cirrhosis, Sjogren's syndrome, temporal arteritis, and Wegener's granulomatosis. The term “inflammatory disorders” includes diseases or disorders involving acute or chronic inflammation such as allergies, asthma, atopic dermatitis, prostatitis, glomerulonephritis, pelvic inflammatory disease (PID), inflammatory bowel disease (IBD, e.g., Crohn's disease, ulcerative colitis), reperfusion injury, rheumatoid arthritis, transplant rejection, and vasculitis. In some embodiments, the present invention provides a method of treating rheumatoid arthritis. In some embodiments, the present invention provides a method of treating multiple sclerosis. In some embodiments, the present invention provides a method of treating systemic lupus erythematosus. In some embodiments, the present invention provides a method of treating atopic dermatitis.

The term “cancer” includes diseases or disorders involving abnormal cell growth and/or proliferation, such as glioma, thyroid carcinoma, breast carcinoma, lung cancer (e.g. small-cell lung carcinoma, non-small-cell lung carcinoma), gastric carcinoma, gastrointestinal stromal tumors, pancreatic carcinoma, bile duct carcinoma, ovarian carcinoma, endometrial carcinoma, prostate carcinoma, renal cell carcinoma, lymphoma (e.g., anaplastic large-cell lymphoma), leukemia (e.g. acute myeloid leukemia, T-cell leukemia, chronic lymphocytic leukemia), multiple myeloma, malignant mesothelioma, malignant melanoma, and colon cancer (e.g. microsatellite instability-high colorectal cancer). In some embodiments, the present invention provides a method of treating leukemia or lymphoma.

As used herein, the term “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

As used herein, the term “treating” or “treatment” refers to obtaining desired pharmacological and/or physiological effect. The effect can be therapeutic, which includes achieving, partially or substantially, one or more of the following results: partially or totally reducing the extent of the disease, disorder or syndrome; ameliorating or improving a clinical symptom or indicator associated with the disorder; or delaying, inhibiting or decreasing the likelihood of the progression of the disease, disorder or syndrome.

The effective dose of a compound provided herein, or a pharmaceutically acceptable salt thereof, administered to a subject can be 10 μg −500 mg.

Administering a compound described herein, or a pharmaceutically acceptable salt thereof, to a mammal comprises any suitable delivery method. Administering a compound described herein, or a pharmaceutically acceptable salt thereof, to a mammal includes administering a compound described herein, or a pharmaceutically acceptable salt thereof, topically, enterally, parenterally, transdermally, transmucosally, via inhalation, intracisternally, epidurally, intravaginally, intravenously, intramuscularly, subcutaneously, intradermally or intravitreally to the mammal. Administering a compound described herein, or a pharmaceutically acceptable salt thereof, to a mammal also includes administering topically, enterally, parenterally, transdermally, transmucosally, via inhalation, intracisternally, epidurally, intravaginally, intravenously, intramuscularly, subcutaneously, intradermally or intravitreally to a mammal a compound that metabolizes within or on a surface of the body of the mammal to a compound described herein, or a pharmaceutically acceptable salt thereof.

Thus, a compound or pharmaceutically acceptable salt thereof as described herein, may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the compound or pharmaceutically acceptable salt thereof as described herein may be combined with one or more excipients and used in the form of ingestible tablets, buccal tablets, troches, capsules, elixirs, suspensions, syrups, or wafers, and the like. Such compositions and preparations should contain at least about 0.1% of active compound. The percentage of the compositions and preparations may, of course, be varied and may conveniently be between about 2 to about 60% of the weight of a given unit dosage form. The amount of active compound in such therapeutically useful compositions can be such that an effective dosage level will be obtained.

The tablets, troches, pills, capsules, and the like can include the following: binders such as gum tragacanth, acacia, corn starch or gelatin; excipients such as dicalcium phosphate; a disintegrating agent such as corn starch, potato starch, alginic acid and the like; a lubricant such as magnesium stearate; or a sweetening agent such as sucrose, fructose, lactose or aspartame or a flavoring agent.

The active compound may also be administered intravenously or intraperitoneally by infusion or injection. Solutions of the active compound or its salts can be prepared in water, optionally mixed with a nontoxic surfactant.

Exemplary pharmaceutical dosage forms for injection or infusion can include sterile aqueous solutions or dispersions or sterile powders comprising the active ingredient which are adapted for the extemporaneous preparation of sterile injectable or infusible solutions or dispersions. In all cases, the ultimate dosage form should be sterile, fluid and stable under the conditions of manufacture and storage.

Sterile injectable solutions can be prepared by incorporating the active compound in the required amount in the appropriate solvent with various of the other ingredients enumerated above, as required, followed by filter sterilization. In the case of sterile powders for the preparation of sterile injectable solutions, the preferred methods of preparation can be vacuum drying and the freeze drying techniques, which can yield a powder of the active ingredient plus any additional desired ingredient present in the previously sterile-filtered solutions.

Exemplary solid carriers can include finely divided solids such as talc, clay, microcrystalline cellulose, silica, alumina and the like. Useful liquid carriers include water, alcohols or glycols or water-alcohol/glycol blends, in which the compounds or pharmaceutically acceptable salts thereof as described herein can be dissolved or dispersed at effective levels, optionally with the aid of non-toxic surfactants.

Useful dosages of a compound or pharmaceutically acceptable salt thereof as described herein can be determined by comparing their in vitro activity, and in vivo activity in animal models. Methods for the extrapolation of effective dosages in mice, and other animals, to humans are known to the art; for example, see U.S. Pat. No. 4,938,949, which is incorporated by reference in its entirety.

The amount of a compound or pharmaceutically acceptable salt thereof as described herein, required for use in treatment can vary not only with the particular salt selected but also with the route of administration, the nature of the condition being treated and the age and condition of the patient and can be ultimately at the discretion of the attendant physician or clinician. In general, however, a dose can be in the range of from about 0.1 to about 10 mg/kg of body weight per day.

The a compound or pharmaceutically acceptable salt thereof as described herein can be conveniently administered in unit dosage form; for example, containing 0.01 to 10 mg, or 0.05 to 1 mg, of active ingredient per unit dosage form. In some embodiments, a dose of 5 mg/kg or less can be suitable.

The desired dose may conveniently be presented in a single dose or as divided doses administered at appropriate intervals.

The disclosed method can include a kit comprising a compound or pharmaceutically acceptable salt thereof as described herein and instructional material which can describe administering a compound or pharmaceutically acceptable salt thereof as described herein or a composition comprising a compound or pharmaceutically acceptable salt thereof as described herein to a cell or a subject. This should be construed to include other embodiments of kits that are known to those skilled in the art, such as a kit comprising a (such as sterile) solvent for dissolving or suspending a compound or pharmaceutically acceptable salt thereof as described herein or composition prior to administering a compound or pharmaceutically acceptable salt thereof as described herein or composition to a cell or a subject. In some embodiments, the subject can be a human.

### Exemplification

Synthesis of compounds of Formulas (I), (IIA), (IIB), (IIIA), (IIIB), (IVA), (IVB), (VA), (VB), (VIA), (VIB), (VIIA), (VIIB), (VIIIA), (VIIIB), (IXA), (IXB), (XA), (XB), (XIA), (XIB), (XIIA), or (XIIB):
Abbreviations and acronyms used herein include the following:
AcOH means acetic acid;
Aq. means aqueous;
Ar means argon;
Bn means benzyl;
Boc means tert-butoxy carbonyl;
Boc₂O means di-tert-butyl dicarbonate;
(BPin)₂ means bis(pinacolato)diboron;
br means broad;
n-BuOH means n-butanol;
t-BuOH means tert-butanol;
n-BuLi means n-butyl lithium;
° C. means degrees Celsius;
CCl₄ means carbon tetrachloride;
CHCl₃ means chloroform;
CDCl₃ means deutero-chloroform;
CDI means 1,1′-carbonyldiimidazole;
CO means carbon monoxide;
CO₂ means carbon dioxide;
Cs₂CO₃ means cesium carbonate;
CuBr means copper bromide;
CuCN means copper cyanide;
CuI means copper iodide;
S means chemical shift;
d means doublet;
dd means double doublet;
DCM means dichloromethane;
DAST means (diethylamino)sulfur trichloride;
DIPEA means N-ethyldiisopropylamine or N,N-diisopropylethylamine;
DEA means diethylamine;
DIAD means diisopropyl azodiformate;
DMAP means 4-(dimethlamino)pyridine;
DMF means N,N-dimethylformamide;
DMSO means dimethylsulfoxide;
DMSO-d₆ means hexadeuterodimethyl sulfoxide;
D₂O means deuterated water;
EDC means N-(3-dimethylaminopropyl)-N′-ethylcarbodiimide hydrochloride;
Et means ethyl;
Et₂O means ether;
EtOH means ethanol;
EtOAc means ethyl acetate;
Eq. means equivalent;
g means gram;
HATU means N-[(dimethylamino)-1H-1,2,3-triazolo-[4,5-b]pyridin-1-ylmethylene]-N-methylmethanaminium hexafluorophosphate N-oxide;
HBr means hydrogen bromide;
HCl means hydrochloric acid;
HCO₂H means formic acid;
Hept means heptanes;
¹H NMR means proton nuclear magnetic resonance;
H₂O means water;
HPLC means high pressure liquid chromatography;
h means hour;
IPA means isopropyl alcohol;
K₂CO₃ means potassium carbonate;
KF means potassium fluoride;
KI means potassium iodide;
KOAc means potassium acetate;
KOtBu means potassium tert-butoxide;
KOH means potassium hydroxide;
K₃PO₄ means potassium phosphate tribasic;
L means liter;
LCMS means liquid chromatography mass spectrometry;
LiGH means lithium hydroxide;
m means multiplet;
M means molar;
Me means methyl;
MeCN means acetonitrile;
MeI means methyl iodide;
MeOH means methanol;
MeOH-d₄ means deutero-methanol;
mg means milligram;
MgSO₄ means magnesium sulfate;
MHz means mega Hertz;
mins means minutes;
mL means milliliters;
mmol means millimole;
MnO₂ means manganese (IV) oxide;
MS m/z means mass spectrum peak;
MV means Mass volume ratio;
N₂ means nitrogen;
Na₂CO₃ means sodium carbonate;
NaH means sodium hydride;
NaHCO₃ means sodium bicarbonate;
NaI means sodium iodide;
NaOH means sodium hydroxide;
NaOtBu means sodium tert-butoxide;
Na₂SO₃ means sodium thiosulfate;
Na₂SO₄ means sodium sulfate;
NBS means N-bromosuccinimide;
NH₃ means ammonia;
NH₄Cl means ammonium chloride;
NH₄HCO₃ means ammonium bicarbonate;
NH₄OH is ammonium hydroxide;
Pd/C means palladium on carbon;
Pd(t-Bu₃P)₂ means bis(tri-tert-butylphosphine)palladium(0);
P(t-Bu)₃Pd G2 means chloro[(tri-tert-butylphosphine)-2-(2-aminobiphenyl)] palladium(II);
Pd(OAc)₂ means palladium acetate;
Pd₂(dba)₃ means tris(dibenzylideneacetone)dipalladium (0);
Pd(dppf)Cl₂ means [1,1′-bis(diphenylphosphino)ferrocene]dichloropalladium(II);
Pd(dppf)Cl₂•DCM means [1,1′-bis(diphenylphosphino)ferrocene]dichloropalladium(II), complex
with dichloromethane;
Pd(dtbpf)Cl₂ means [1,1′-bis(di-tert-butylphosphino)ferrocene]dichloropalladium(II);
Pd(PPh₃)₄ means tetrakis(triphenylphosphine)palladium(0);
PE means petroleum ether;
POCl₃ means phosphoryl chloride;
i-PrOH means isopropanol;
PPh₃ means triphenylphosphine;
PyBroP means bromotripyrrolidinophosphonium hexafluorophosphate;
q means quartet;
Rt means retention time;
rt means room temperature;
RuPhos means 2-dicyclohexylphosphino-2′,6′-diisopropoxybiphenyl;
RuPhos Pd G3 means (2-dicyclohexylphosphino-2′,6′-diisopropoxy-1,1′-biphenyl)[2-(2′-amino-1,1′-biphenyl)]palladium(II) methanesulfonate;
s means singlet;
sat. means saturated;
SCX means strong cation exchange;
SFC means supercritical fluid chromatography;
SiO₂ means silicon dioxide;
SOCl₂ means thionyl chloride;
soln. means solution;
t means triplet;
TBDMS means tert-butyldimethylsilyl;
TBME means tert-butyl methyl ether;
TEA means triethylamine;
TFA means trifluoroacetic acid;
THF means tetrahydrofuran;
TLC means thin layer chromatography;
TMOS means tetramethyl orthosilicate;
TMS means trimethylsilyl;
T3P means 2,4,6-tripropyl-1,3,5,2,4,6-trioxatriphosphorinane-2,4,6-trioxide;
TTBP means tri-tert-butylphosphonium tetrafluoroborate;
μL means micro liters;
μmol means micromole;
   Preparative HPLC conditions

In the Example sections below, the following preparative HPLC methods were used.

### Method A1:

Column: Welch Xtimate C18 150×25 mm×5 μm
Mobile phase A: MeCN
Mobile phase B: H₂O
Modifier: 10 mM NH₄HCO₃
Gradient (% organic): % optimized for each example
Flow rate: 25 mL/min
Gradient time: 10 min

### Method A2:

Column: Welch Xtimate CSH C18 150×50 mm×5 μm
Mobile phase A: MeCN
Mobile phase B: H₂O
Modifier: NH₄OH, 10-90%
Gradient (% organic): optimized for each example

### Method B:

Column: Agela Durashell C18 50×30 mm, 5 μm
Mobile phase A: MeCN
Mobile phase B: H₂O
Modifier: 0.05% NH₄OH+10 mM NH₄HCO₃
Gradient (% organic): optimized for each example.
Flow rate: 25 mL/min

### Method C:

Column: Waters XSelect CSH OBD C18 150×19 mm, 5 μm
Mobile phase A: MeCN
Mobile phase B: H₂O
Modifier: NH₃
Gradient (% organic): optimized for each example.

### Preparative SFC Conditions

### Method D:

Column: CHIRALPAK IB 3×250 mm, 5 μm
Mobile phase B: 30% EtOH w/0.1% DEA in CO₂
Flow rate: 100 mL/min
ABPR 120 bar, MBPR 40 psi, column temp 40° C.

### Common Intermediates

### Intermediate 1: 1-(tert-butyl)-N-(2-methyl-4-(4,4,5,5-tetramethyl-1,3,2-dioxaborolan-2-yl)benzyl)-1H-1,2,3-triazole-4-carboxamide

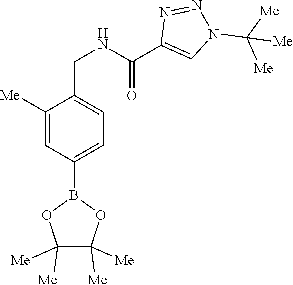

### 1. Synthesis of N-(4-bromo-2-methylbenzyl)-1-(tert-butyl)-1H-1,2,3-triazole-4-carboxamide

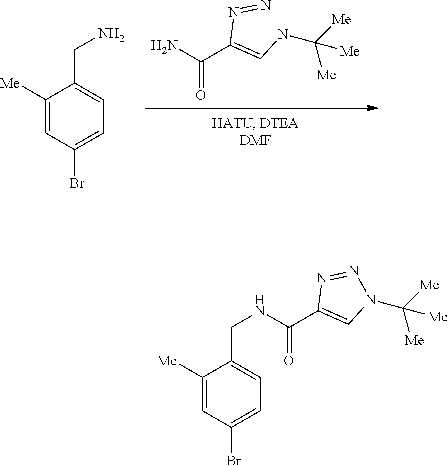 [SMILES: Cc1cc(Br)ccc1CN ; CC(C)(C)N/C=C(\N=N)C(N)=O ; Cc1cc(Br)ccc1CNC(=O)c1cn(C(C)(C)C)nn1]

To a solution of 1-(tert-butyl)-1H-1,2,3-triazole-4-carboxylic acid (450 mg, 2.66 mmol), (4-bromo-2-methylphenyl)methanamine (585 mg, 2.93 mmol) and HATU (1.2 g, 3.19 mmol) in DMF (6.0 mL) was added TEA (1.1 mL, 7.98 mmol) and the reaction stirred at 25° C. for 2 h. The reaction was concentrated in vacuo and the residue extracted with EtOAc (20 mL×3). The combined organic layers were dried over Na₂SO₄, filtered, and the filtrate was concentrated in vacuo. The crude product was purified by silica gel chromatography eluting with PE/EtOAc (1/1 to 1/9) to give N-(4-bromo-2-methylbenzyl)-1-(tert-butyl)-1H-1,2,3-triazole-4-carboxamide (860 mg, 92% yield) as a white solid. LCMS m/z=352.8 (M+H)+.

### 2. Synthesis of 1-(tert-butyl)-N-(2-methyl-4-(4,4,5,5-tetramethyl-1,3,2-dioxaborolan-2-yl)benzyl)-1H-1,2,3-triazole-4-carboxamide

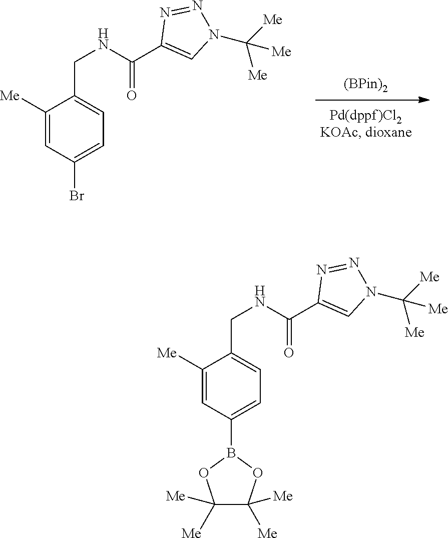 [SMILES: Cc1cc(Br)ccc1CNC(=O)c1cn(C(C)(C)C)nn1 ; Cc1cc(B2OC(C)(C)C(C)(C)O2)ccc1CNC(=O)c1cn(C(C)(C)C)nn1]

N₂ was bubbled through a suspension of N-(4-bromo-2-methylbenzyl)-1-(tert-butyl)-1H-1,2,3-triazole-4-carboxamide (860 mg, 2.45 mmol), (BPin)₂ (1.5 g, 6.13 mmol) and KOAc (601 mg, 6.13 mmol) in dioxane (20 mL). Pd(dppf)Cl₂ (179.2 mg, 0.245 mmol) was added and the reaction was stirred at 90° C. for 2 h under N₂. The reaction mixture was concentrated in vacuo and the residue was purified by silica gel chromatography eluting with PE/EtOAc (3/1) to give 1-(tert-butyl)-N-(2-methyl-4-(4,4,5,5-tetramethyl-1,3,2-dioxaborolan-2-yl)benzyl)-1H-1,2,3-triazole-4-carboxamide (900 mg, 92% yield) as a white solid. LCMS m/z=398.9 (M+H)+.

### Intermediate 2: 3-(tert-butyl)-N-(2-methyl-4-(4,4,5,5-tetramethyl-1,3,2-dioxaborolan-2-yl)benzyl)-1,2,4-oxadiazole-5-carboxamide

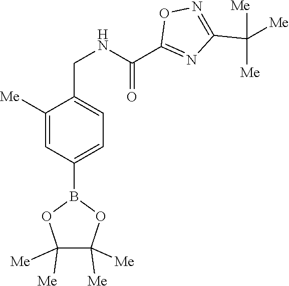

### 1. Synthesis of N-(4-bromo-2-methylbenzyl)-1-(tert-butyl)-1,2,4-oxadiazole-5-carboxamide

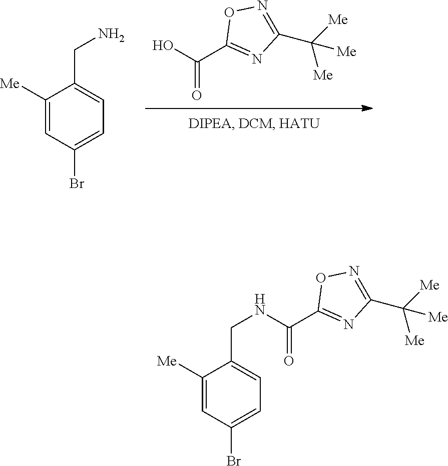 [SMILES: Cc1cc(Br)ccc1CN ; CC(C)(C)c1noc(C(=O)O)n1 ; Cc1cc(Br)ccc1CNC(=O)c1nc(C(C)(C)C)no1]

3-(tert-Butyl)-1,2,4-oxadiazole-5-carboxylic acid (735 mg, 3.56 mmol) and HATU (1.45 g, 3.80 mmol) were slowly added to a solution of (4-bromo-2-methylphenyl)-methanamine (600 mg, 2.54 mmol) and DIPEA (983 mg, 7.61 mmol) in DCM (50 mL) and the reaction was stirred at 20° C. for 1 h. The mixture was concentrated in vacuo, and the crude was purified by silica gel column chromatography eluting with PE/EtOAc (1/0 to 1/1) to give N-(4-bromo-2-methylbenzyl)-1-(tert-butyl)-1,2,4-oxadiazole-5-carboxamide (553 mg, 61% yield) as a yellow solid. LCMS m/z=351.4 (M+H)+.

### 2. Synthesis of 1-(tert-butyl)-N-(2-methyl-4-(4,4,5,5-tetramethyl-1,3,2-dioxaborolan-2-yl)benzyl)-1,2,4-oxadiazole-5-carboxamide

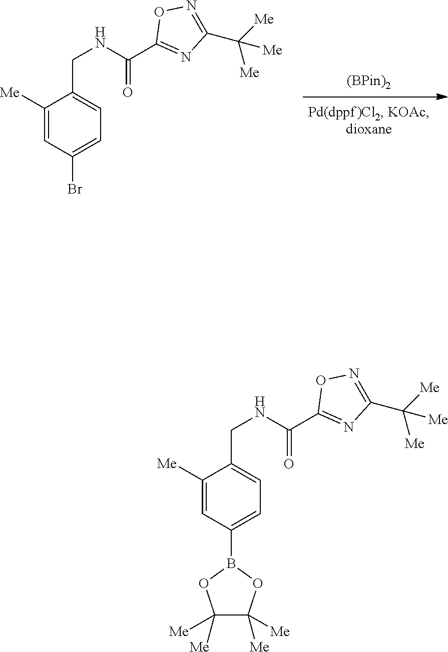 [SMILES: Cc1cc(Br)ccc1CNC(=O)c1nc(C(C)(C)C)no1 ; Cc1cc(B2OC(C)(C)C(C)(C)O2)ccc1CNC(=O)c1nc(C(C)(C)C)no1]

1-(tert-Butyl)-N-(2-methyl-4-(4,4,5,5-tetramethyl-1,3,2-dioxaborolan-2-yl)benzyl)-1,2,4-oxadiazole-5-carboxamide was obtained as a yellow solid from N-(4-bromo-2-methylbenzyl)-1-(tert-butyl)-1,2,4-oxadiazole-5-carboxamide following a similar procedure to that described for Intermediate 1, step 2: 1-(tert-butyl)-N-(2-methyl-4-(4,4,5,5-tetramethyl-1,3,2-dioxaborolan-2-yl)benzyl)-1H-1,2,3-triazole-4-carboxamide (365 mg, 46% yield). LCMS m/z=400.3 (M+H)+.

### Intermediate 3: 5-(tert-butyl)-N-(2-methyl-4-(4,4,5,5-tetramethyl-1,3,2-dioxaborolan-2-yl)benzyl)isoxazole-3-carboxamide

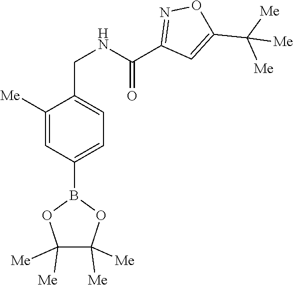 [SMILES: Cc1cc(B2OC(C)(C)C(C)(C)O2)ccc1CNC(=O)c1cc(C(C)(C)C)on1]

### 1. Synthesis of N-(4-bromo-2-methylbenzyl)-5-(tert-butyl)isoxazole-3-carboxamide Br

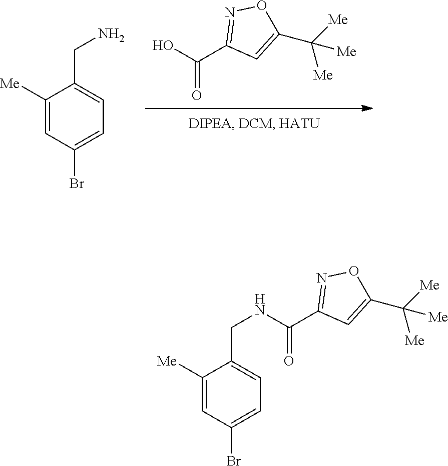 [SMILES: Cc1cc(Br)ccc1CN ; CC(C)(C)c1cc(C(=O)O)no1 ; Cc1cc(Br)ccc1CNC(=O)c1cc(C(C)(C)C)on1]

HATU (1.43 g, 3.75 mmol) was added slowly to a mixture of (4-bromo-2-methyl-phenyl)methanamine (500 mg, 2.50 mmol), DIPEA (646 mg, 5.0 mmol) and 5-tert-butylisoxazole-3-carboxylic acid (381 mg, 2.25 mmol) in DCM (30 mL) and the reaction was stirred at 20° C. for 1 h. The mixture was concentrated in vacuo and the residue purified by silica gel column chromatography eluting with PE/EtOAc (1/0 to 5/1) to give N-(4-bromo-2-methylbenzyl)-5-(tert-butyl)isoxazole-3-carboxamide (617 mg, 68% yield) as a yellow solid. LCMS m/z=351.1 (M+H)+.

### 2. Synthesis of 5-(tert-butyl)-N-(2-methyl-4-(4,4,5,5-tetramethyl-1,3,2-dioxaborolan-2-yl)benzyl)isoxazole-3-carboxamide

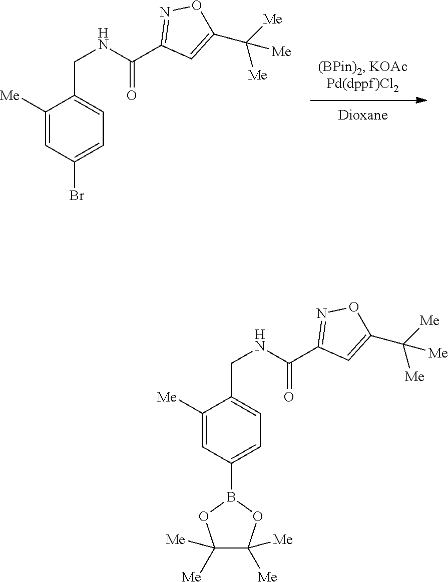 [SMILES: Cc1cc(Br)ccc1CNC(=O)c1cc(C(C)(C)C)on1 ; Cc1cc(B2OC(C)(C)C(C)(C)O2)ccc1CNC(=O)c1cc(C(C)(C)C)on1]

5-(tert-Butyl)-N-(2-methyl-4-(4,4,5,5-tetramethyl-1,3,2-dioxaborolan-2-yl)benzyl)isoxazole-3-carboxamide was obtained as a yellow solid from N-(4-bromo-2-methylbenzyl)-5-(tert-butyl)isoxazole-3-carboxamide following a similar procedure to that described in Intermediate 1, step 2: 1-(tert-butyl)-N-(2-methyl-4-(4,4,5,5-tetramethyl-1,3,2-dioxaborolan-2-yl)benzyl)-1H-1,2,3-triazole-4-carboxamide, (600 mg, 81% yield). LCMS m/z=399.3 (M+H)+.

### Intermediate 4: N-methyl-N-(2-methyl-4-(4,4,5,5-tetramethyl-1,3,2-dioxaborolan-2-yl)benzyl)-6-(trifluoromethyl)nicotinamide

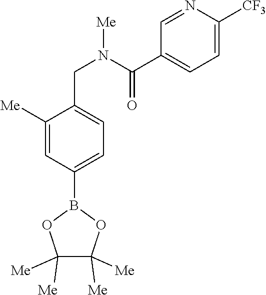 [SMILES: Cc1cc(B2OC(C)(C)C(C)(C)O2)ccc1CN(C)C(=O)c1ccc(C(F)(F)F)nc1]

### 1. Synthesis of N-(4-bromo-2-methylbenzyl)-N-methyl-6-(trifluoromethyl)nicotinamide

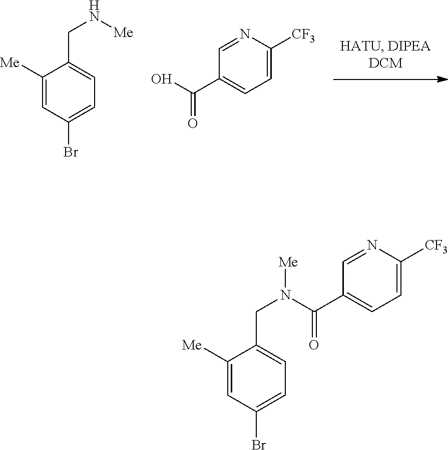 [SMILES: CNCc1ccc(Br)cc1C ; O=C(O)c1ccc(C(F)(F)F)nc1 ; Cc1cc(Br)ccc1CN(C)C(=O)c1ccc(C(F)(F)F)nc1]

A mixture of 1-(4-bromo-2-methyl-phenyl)-N-methyl-methanamine (300 mg, 1.40 mmol), 6-(trifluoromethyl)pyridine-3-carboxylic acid (268 mg, 1.40 mmol), HATU (1.07 g, 2.80 mmol) and DIPEA (543 mg, 4.20 mmol) in DCM (5 mL) was stirred at rt for 16 h. The reaction was filtered through Celite® and washed with DCM. The filtrate was concentrated in vacuo and the residue was purified by silica gel column chromatography eluting with heptanes/EtOAc (100/0 to 1/1) to give N-(4-bromo-2-methylbenzyl)-N-methyl-6-(trifluoromethyl)nicotinamide (527 mg, 92% yield) as a white solid. LCMS m/z=389.0 (M+H)+.

### 2. Synthesis of N-methyl-N-(2-methyl-4-(4,4,5,5-tetramethyl-1,3,2-dioxaborolan-2-yl)benzyl)-6-(trifluoromethyl)nicotinamide

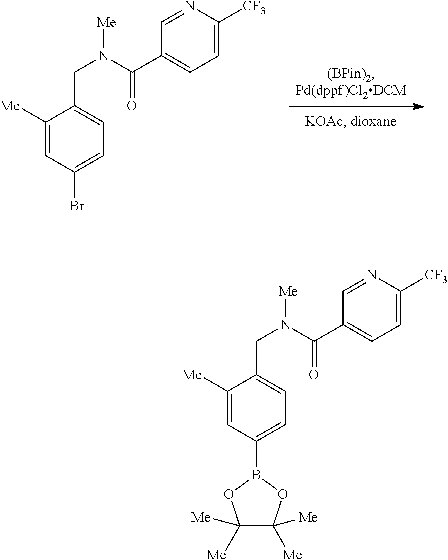 [SMILES: Cc1cc(Br)ccc1CN(C)C(=O)c1ccc(C(F)(F)F)nc1 ; Cc1cc(B2OC(C)(C)C(C)(C)O2)ccc1CN(C)C(=O)c1ccc(C(F)(F)F)nc1]

A mixture of N-(4-bromo-2-methylbenzyl)-N-methyl-6-(trifluoromethyl)nicotinamide (527 mg, 1.36 mmol), (BPin)₂ (346 mg, 1.36 mmol), KOAc (401 mg, 4.08 mmol) and Pd(dppf)Cl₂•DCM (111 mg, 136 μmol) in dioxane (5 mL) was degassed and heated at 95° C. for 16 h. The cooled mixture was diluted with EtOAc and filtered through Celite®. The filtrate was concentrated in vacuo and the residue purified by silica gel column chromatography eluting with heptanes/EtOAc (100/0 to 0/100) to give N-methyl-N-(2-methyl-4-(4,4,5,5-tetramethyl-1,3,2-dioxaborolan-2-yl)benzyl)-6-(trifluoromethyl)nicotinamide (523 mg, 89% yield) as a gel. LCMS m/z=435.2 (M+H)+.

### Intermediate 5: 5-(tert-butyl)-N-(2-methyl-4-(4,4,5,5-tetramethyl-1,3,2-dioxaborolan-2-yl)benzyl)-1,2,4-oxadiazole-3-carboxamide

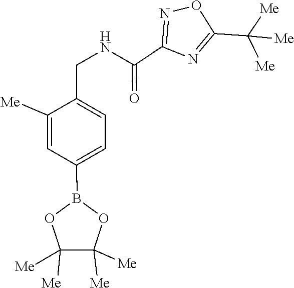 [SMILES: Cc1cc(B2OC(C)(C)C(C)(C)O2)ccc1CNC(=O)c1noc(C(C)(C)C)n1]

### 1. Synthesis of (2-methyl-4-(4,4,5,5-tetramethyl-1,3,2-dioxaborolan-2-yl)phenyl)methanamine hydrochloride

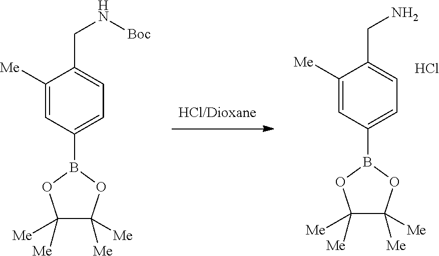 [SMILES: Cc1cc(B2OC(C)(C)C(C)(C)O2)ccc1CNC(=O)OC(C)(C)C ; Cc1cc(B2OC(C)(C)C(C)(C)O2)ccc1CN ; Cl]

A solution of tert-butyl (2-methyl-4-(4,4,5,5-tetramethyl-1,3,2-dioxaborolan-2-yl)benzyl)carbamate (5.00 g, 14.4 mmol) in 4M HCl/dioxane (100 mL) was stirred at 20° C. for 1 h. The mixture was evaporated under reduced pressure to give (2-methyl-4-(4,4,5,5-tetramethyl-1,3,2-dioxaborolan-2-yl)phenyl)methanamine hydrochloride as a light yellow solid (4.0 g, 98% yield), which was carried forward without further purification.

### 2. Synthesis of 5-(tert-butyl)-N-(2-methyl-4-(4,4,5,5-tetramethyl-1,3,2-dioxaborolan-2-yl)benzyl)-1,2,4-oxadiazole-3-carboxamide

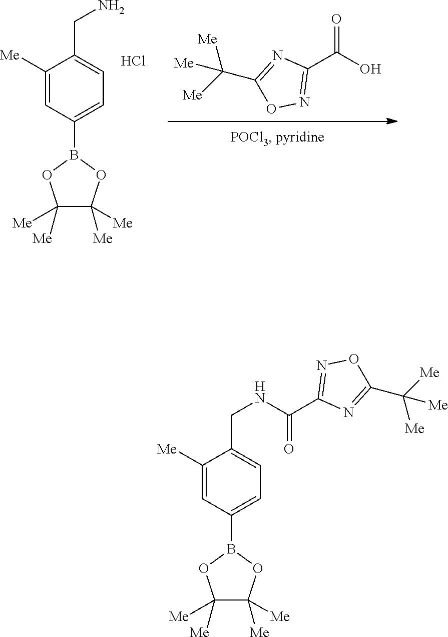 [SMILES: Cc1cc(B2OC(C)(C)C(C)(C)O2)ccc1CN ; Cl ; CC(C)(C)c1nc(C(=O)O)no1 ; Cc1cc(B2OC(C)(C)C(C)(C)O2)ccc1CNC(=O)c1noc(C(C)(C)C)n1]

To a solution of (2-methyl-4-(4,4,5,5-tetramethyl-1,3,2-dioxaborolan-2-yl)phenyl)methanamine hydrochloride (3.33 g, 11.8 mmol) in DCM (20 mL) was added 5-(tert-butyl)-1,2,4-oxadiazole-3-carboxylic acid (2.00 g, 11.8 mmol) and the solution was cooled to 0° C. POCl₃ (3.28 mL, 35.3 mmol) was added, followed by pyridine (5.69 mL, 70.5 mmol) and the reaction was stirred at 20° C. for 1 h. The reaction was poured into sat. aq. NaHCO₃ solution (300 mL) and the mixture extracted with DCM (300 mL). The organic layer was washed with brine (200 mL), dried (Na₂SO₄), filtered, and concentrated in vacuo. The residue was purified by silica gel column chromatography eluting with PE/EtOAc (5/1 to 1/1) to give 5-(tert-butyl)-N-(2-methyl-4-(4,4,5,5-tetramethyl-1,3,2-dioxaborolan-2-yl)benzyl)-1,2,4-oxadiazole-3-carboxamide as a light yellow oil (3.4 g, 73% yield). ¹H NMR (400 MHz CDCl₃) δ: 7.69-7.64 (m, 2H), 7.32 (d, 1H), 7.07 (br s, 1H), 4.68 (d, 2H), 2.38 (s, 3H), 1.46 (s, 9H), 1.35 (s, 12H).

### Intermediate 6:3-(difluoromethyl)-1-methyl-N-(2-methyl-4-(4,4,5,5-tetramethyl-1,3,2-dioxaborolan-2-yl)benzyl)-1H-pyrazole-4-carboxamide

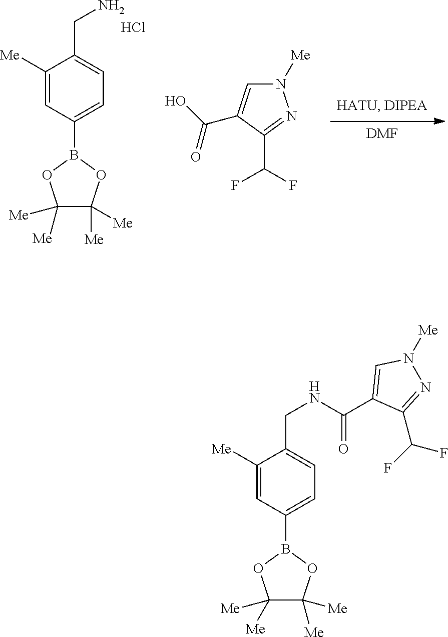

DIPEA (953 μL, 5.46 mmol) was added to Intermediate 5, step 1:(2-methyl-4-(4,4,5,5-tetramethyl-1,3,2-dioxaborolan-2-yl)phenyl)methanamine hydrochloride (500 mg, 1.76 mmol) in DMF (10 mL) and the solution was stirred for 5 mins at rt. 3-(Difluoromethyl)-1-methyl-pyrazole-4-carboxylic acid (620 mg, 3.52 mmol) and HATU (671 mg, 1.76 mmol) were added and the reaction was stirred for 4 h at rt. The reaction mixture was partitioned between water and EtOAc and the layers were separated. The aqueous phase was extracted with EtOAc (2×), and the combined organic layers were washed with brine (50 mL) and concentrated in vacuo. The crude was purified by silica gel column chromatography eluting with EtOAc/heptanes (0/100 to 6/4) to give 3-(difluoromethyl)-1-methyl-N-(2-methyl-4-(4,4,5,5-tetramethyl-1,3,2-dioxaborolan-2-yl)benzyl)-1H-pyrazole-4-carboxamide (450 mg, 63% yield). LCMS m/z=406.2 (M+H)+.

### Intermediate 7: 5-(tert-butyl)-N-(3-fluoro-2-methyl-4-(4,4,5,5-tetramethyl-1,3,2-dioxaborolan-2-yl)benzyl)-1,2,4-oxadiazole-3-carboxamide

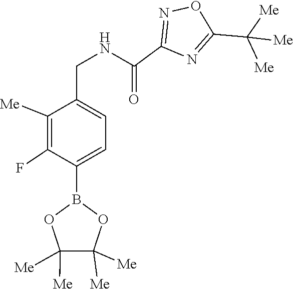 [SMILES: Cc1c(CNC(=O)c2noc(C(C)(C)C)n2)ccc(B2OC(C)(C)C(C)(C)O2)c1F]

### 1. Synthesis of 4-bromo-2-fluoro-3-methylaniline

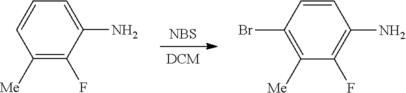

A solution of NBS (28.4 g, 160 mmol) in DCM was added dropwise to an ice-cooled solution of 2-fluoro-3-methylaniline (20.0 g, 160 mmol) in DCM (200 mL) and the reaction was stirred at 20° C. for 5 h. The mixture was diluted with sat. aq. Na₂CO₃ solution (100 mL), the layers separated, and the aqueous phase was extracted with DCM (2×100 mL). The combined organic layers were dried (Na₂SO₄), filtered, and concentrated in vacuo. The crude product was purified by silica gel column chromatography eluting with PE/EtOAc (10/1) to give 4-bromo-2-fluoro-3-methylaniline as a brown oil (32.0 g, crude). LCMS m/z=205.8 (M+H)+.

### 2. Synthesis of 4-amino-3-fluoro-2-methylbenzonitrile

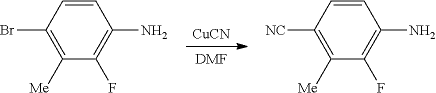

CuCN (35.6 g, 397 mmol) was added to a solution of 4-bromo-2-fluoro-3-methylaniline (27 g, 132 mmol) in DMF (200 mL) under N₂ and the reaction was stirred at 140° C. for 16 h. NH₃•H₂O (300 mL) was added to the cooled reaction, the mixture was filtered, and the filtrate was poured into H₂O (300 mL) and extracted with EtOAc (2×300 mL). The aqueous phase was extracted with EtOAc (2×300 mL) and the combined organic layers were dried (Na₂SO₄), filtered, and concentrated in vacuo. The crude product was purified by silica gel column chromatography eluting with PE/EtOAc (70/30) to give 4-amino-3-fluoro-2-methylbenzonitrile as a brown oil (15 g, 75% yield). LCMS m/z=151.0 (M+H)+.

### 3. Synthesis of 4-bromo-3-fluoro-2-methylbenzonitrile

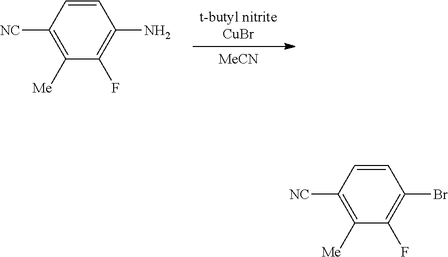 [SMILES: "MeCN" ; Cc1c(C#N)ccc(N)c1F ; "t-butyl nitriteCuBr" ; Cc1c(C#N)ccc(Br)c1F]

A solution of 4-amino-3-fluoro-2-methylbenzonitrile (15 g, 100 mmol) in MeCN (250 mL) was added to a solution of tert-butyl nitrite (17.8 mL, 150 mmol) and CuBr (21.5 g, 150 mmol) in MeCN at 65° C. and the reaction was stirred at 65° C. for 3 h under N₂. The cooled mixture was filtered, the filtrate was concentrated in vacuo, and the crude product purified by silica gel column chromatography eluting with PE/EtOAc (9/1) to give 4-bromo-3-fluoro-2-methylbenzonitrile as an orange solid (11.5 g, crude). ¹H NMR (400 MHz, CDCl₃) δ: 7.51 (dd, 1H), 7.28 (dd, 1H), 2.52 (s, 3H).

### 4. Synthesis of (4-bromo-3-fluoro-2-methylphenyl)methanamine

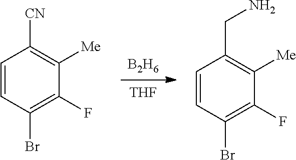

To a solution of 4-bromo-3-fluoro-2-methylbenzonitrile (15.0 g, 70 mmol) in THF (150 mL) was added B₂H₆ (10.5 mL, 105 mmol, 10 M in Me₂S) at 25° C. slowly. The reaction mixture was heated at 65° C. for 17 h. The mixture was quenched with MeOH (10 mL) and concentrated in vacuo to give crude (4-bromo-3-fluoro-2-methylphenyl)methanamine (15 g, crude), which was used for the next step directly without further purification.

### 5. Synthesis of tert-butyl (4-bromo-3-fluoro-2-methylbenzyl)carbamate

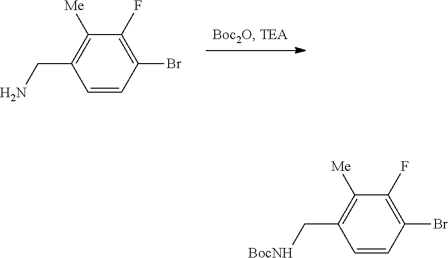 [SMILES: Cc1c(CN)ccc(Br)c1F ; "Boc2O, TEA" ; Cc1c(CNC(=O)OC(C)(C)C)ccc(Br)c1F]

To a solution of (4-bromo-3-fluoro-2-methylphenyl)methanamine (14 g, 64 mmol) in DCM (100 mL) were added TEA (13 g, 128 mmol) and Boc₂O (16.8 g, 77 mmol) and the mixture was stirred at 25° C. for 2 h. The mixture was concentrated in vacuo and the residue was purified by silica gel column chromatography PE/EtOAc (50/1) to give tert-butyl (4-bromo-3-fluoro-2-methylbenzyl)carbamate as a white solid (12.0 g, 59%). ¹H NMR (400 MHz, DMSO-d₆) δ: 7.47 (dd, 1H), 7.37 (dd, 1H), 6.96 (d, 1H), 4.07 (d, 2H), 2.19 (d, 3H), 1.37 (s, 9H).

### 6. Synthesis of tert-butyl (3-fluoro-2-methyl-4-(4,4,5,5-tetramethyl-1,3,2-dioxaborolan-2-yl)benzyl)carbamate

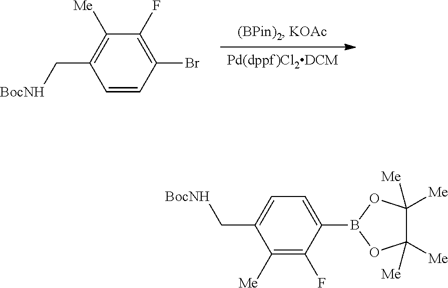 [SMILES: Cc1c(CNC(=O)OC(C)(C)C)ccc(Br)c1F ; "(BPin)2, KOAc" ; "Pd(dppf)Cl2·DCM" ; Cc1c(CNC(=O)OC(C)(C)C)ccc(B2OC(C)(C)C(C)(C)O2)c1F]

To a solution of tert-butyl (4-bromo-3-fluoro-2-methylbenzyl)carbamate (10 g, 31 mmol) in dioxane (150 mL) were added (BPin)₂ (9.6 g, 38 mmol) and KOAc (6.2 g, 63 mmol). Pd(dppf)Cl₂•DCM (2.1 g, 2.5 mmol) was added and the reaction was stirred at 80° C. for 17 h under N₂. The reaction mixture was concentrated in vacuo and the crude material was purified by silica-gel column chromatography eluting with PE/EtOAc (20/1) to give tert-butyl (3-fluoro-2-methyl-4-(4,4,5,5-tetramethyl-1,3,2-dioxaborolan-2-yl)benzyl)carbamate as a yellow solid (13.0 g, crude). LCMS m/z=310.1 (M-tBu+H)+.

### 7. Synthesis of (3-fluoro-2-methyl-4-(4,4,5,5-tetramethyl-1,3,2-dioxaborolan-2-yl)phenyl)methanamine hydrochloride

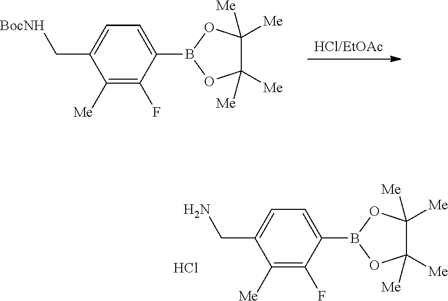 [SMILES: Cc1c(CNC(=O)OC(C)(C)C)ccc(B2OC(C)(C)C(C)(C)O2)c1F ; "HCl/EtOAc" ; Cl ; Cc1c(CN)ccc(B2OC(C)(C)C(C)(C)O2)c1F]

To a solution of tert-butyl (3-fluoro-2-methyl-4-(4,4,5,5-tetramethyl-1,3,2-dioxaborolan-2-yl)benzylcarbamate (7.0 g, 19.16 mmol) in EtOAc (10 mL) and MeOH (5 mL) was slowly added HCl (4 M, 13.2 mL) and the reaction was stirred at 15° C. for 4 h. The mixture was evaporated under reduced pressure to give (3-fluoro-2-methyl-4-(4,4,5,5-tetramethyl-1,3,2-dioxaborolan-2-yl)phenyl)methanamine hydrochloride (6.0 g, crude) as a yellow solid, which was carried forward without further purification. LCMS m/z=266.2 (M+H)+.

### 8. Synthesis of 5-(tert-butyl)-N-(3-fluoro-2-methyl-4-(4,4,5,5-tetramethyl-1,3,2-dioxaborolan-2-yl)benzyl)-1,2,4-oxadiazole-3-carboxamide

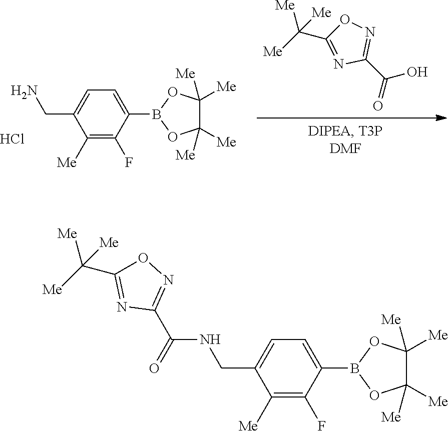 [SMILES: Cl ; Cc1c(CN)ccc(B2OC(C)(C)C(C)(C)O2)c1F ; CC(C)(C)c1nc(C(=O)O)no1 ; "DIPEA, T3PDMF" ; Cc1c(CNC(=O)c2noc(C(C)(C)C)n2)ccc(B2OC(C)(C)C(C)(C)O2)c1F]

DIPEA (2.73 g, 21.2 mmol, 3.7 mL) was added to a suspension of (3-fluoro-2-methyl-4-(4,4,5,5-tetramethyl-1,3,2-dioxaborolan-2-yl)phenyl)methanamine hydrochloride (3.19 g, 10.6 mmol) and 5-(tert-butyl)-1,2,4-oxadiazole-3-carboxylic acid (1.50 g, 7.05 mmol) in anhydrous DMF (20 mL) and the mixture was cooled to 0° C. T3P (5.83 g, 9.2 mmol, 50% purity) was added, the cooling bath was removed and the reaction was stirred at 15° C. for 2 h. The reaction was quenched with water (30 mL) and then EtOAc (30 mL) was added. The layers were separated, and the aqueous layer was extracted with EtOAc (40 mL). The combined organic layers were washed with water (50 mL), sat. aq. NaHCO₃ solution (50 mL), and brine (50 mL). The organic layer was dried over Na₂SO₄, filtered and concentrated in vacuo to give a beige oil. This was purified by silica gel column chromatography (PE/EtOAc, 100/0 to 65/35) to give 5-(tert-butyl)-N-(3-fluoro-2-methyl-4-(4,4,5,5-tetramethyl-1,3,2-dioxaborolan-2-yl)benzyl)-1,2,4-oxadiazole-3-carboxamide (600 mg, 20% yield) as a thick pale-yellow oil, which solidified upon standing. LCMS m/z=417.9 (M+H)+.

### Example 1: 1-(tert-butyl)-N-(2-methyl-4-(3-(3-(N-methylacrylamido)piperidin-1-yl)pyridin-4-yl)benzyl)-1H-1,2,3-triazole-4-carboxamide

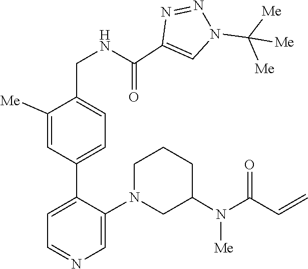 [SMILES: C=CC(=O)N(C)C1CCCN(c2cnccc2-c2ccc(CNC(=O)c3cn(C(C)(C)C)nn3)c(C)c2)C1]

### 1. Synthesis of tert-butyl (1-(4-chloropyridin-3-yl)piperidin-3-yl)(methyl)carbamate

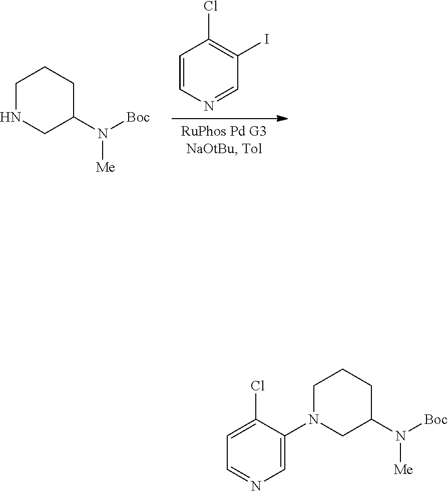

To a solution of 4-chloro-3-iodopyridine (200 mg, 835 μmol) in toluene (5 mL) was added tert-butyl methyl(piperidin-3-yl)carbamate (179 mg, 835 μmol), RuPhos Pd G3 (70 mg, 84 μmol) and NaOt-Bu (241 mg, 2.51 mmol) and the reaction was stirred at 110° C. under N₂ for 10 h. The cooled reaction was concentrated in vacuo and the residue was purified by silica gel chromatography (PE/EtOAc, 1/0 to 0/1) to give tert-butyl (1-(4-chloropyridin-3-yl)piperidin-3-yl)(methyl)carbamate (170 mg, 62% yield) as yellow oil. LCMS m/z=326.3 (M+H)+.

### 2. Synthesis of tert-butyl (1-(4-(4-((1-(tert-butyl)-1H-1,2,3-triazole-4-carboxamido)methyl)-3-methylphenyl)pyridin-3-yl)piperidin-3-yl)(methyl)carbamate

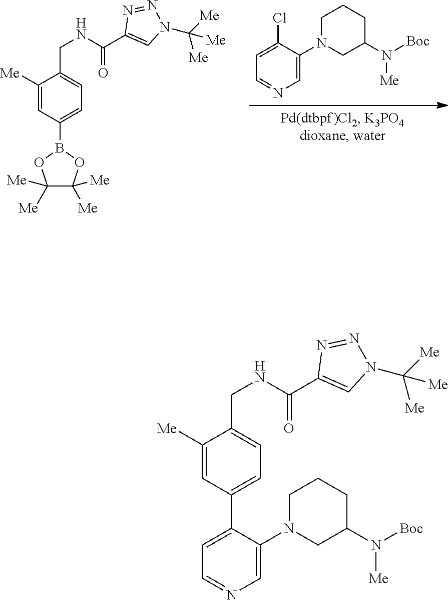 [SMILES: Cc1cc(B2OC(C)(C)C(C)(C)O2)ccc1CNC(=O)c1cn(C(C)(C)C)nn1 ; CN(C(=O)OC(C)(C)C)C1CCCN(c2cnccc2Cl)C1 ; "Pd(dtbpf)Cl2, K3PO4dioxane, water" ; Cc1cc(-c2ccncc2N2CCCC(N(C)C(=O)OC(C)(C)C)C2)ccc1CNC(=O)c1cn(C(C)(C)C)nn1]

To a solution of Intermediate 1: 1-(tert-butyl)-N-(2-methyl-4-(4,4,5,5-tetramethyl-1,3,2-dioxaborolan-2-yl)benzyl)-1H-1,2,3-triazole-4-carboxamide (200 mg, 502 μmol) in dioxane (6 mL) and water (1 mL) was added tert-butyl (1-(4-chloropyridin-3-yl)piperidin-3-yl)(methyl)carbamate (164 mg, 502 μmol), K₃PO₄ (320 mg, 1.51 mmol) and Pd(dtbpf)Cl₂ (33 mg, 50 μmol). The mixture was stirred at 80° C. under N₂ for 2 h, then was allowed to cool and concentrated in vacuo. The residue was purified by silica gel chromatography (PE/EtOAc, 1/0 to 0/1) to give tert-butyl (1-(4-(4-((1-(tert-butyl)-1H-1,2,3-triazole-4-carboxamido)methyl)-3-methylphenyl)pyridin-3-yl)piperidin-3-yl)(methyl)carbamate (120 mg, 43% yield) as colorless oil. LCMS m/z=562.6 (M+H)+.

### 3. Synthesis of 1-(tert-butyl)-N-(2-methyl-4-(3-(3-(methylamino)piperidin-1-yl)pyridin-4-yl)benzyl)-1H-1,2,3-triazole-4-carboxamide hydrochloride

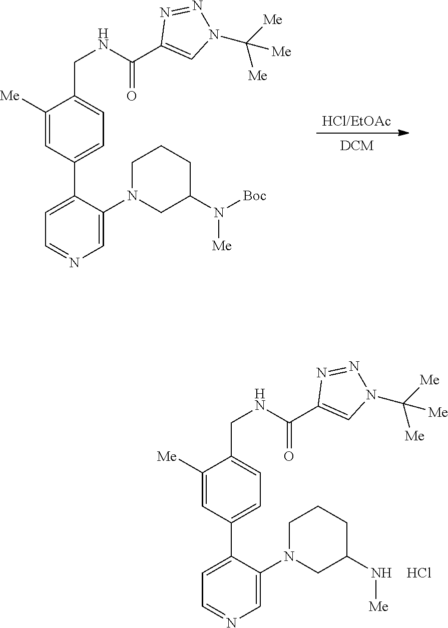

To a solution of tert-butyl (1-(4-(4-((1-(tert-butyl)-1H-1,2,3-triazole-4-carboxamido)methyl)-3-methylphenyl)pyridin-3-yl)piperidin-3-yl)(methyl)carbamate (120 mg, 214 μmol) in DCM (2 mL) was added 4M HCl/EtOAc (15 mL) and the reaction was stirred at 25° C. for 1 h. The mixture was evaporated under reduced pressure to give 1-(tert-butyl)-N-(2-methyl-4-(3-(3-(methylamino)piperidin-1-yl)pyridin-4-yl)benzyl)-1H-1,2,3-triazole-4-carboxamide hydrochloride (100 mg, crude) as a white solid. LCMS m/z=462.4 (M+H)+.

### 4. Synthesis of 1-(tert-butyl)-N-(2-methyl-4-(3-(3-(N-methylacrylamido)piperidin-1-yl)pyridin-4-yl)benzyl)-1H-1,2,3-triazole-4-carboxamide

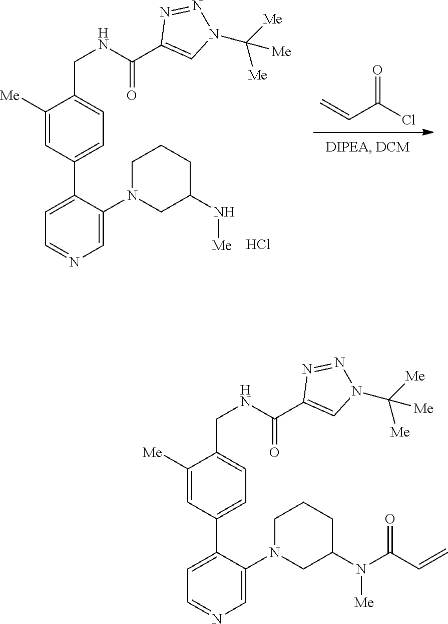

To a solution of 1-(tert-butyl)-N-(2-methyl-4-(3-(3-(methylamino)piperidin-1-yl)pyridin-4-yl)benzyl)-1H-1,2,3-triazole-4-carboxamide hydrochloride (100 mg, 217 μmol) in DCM (20 mL) was added DIPEA (76 μL, 433 μmol) and acryloyl chloride (18 μL, 217 μmol) and the reaction was stirred at 25° C. for 30 mins. The mixture was concentrated in vacuo and the residue was purified by prep-HPLC (Method A2, organic gradient 32-62%), to give 1-(tert-butyl)-N-(2-methyl-4-(3-(3-(N-methylacrylamido)piperidin-1-yl)pyridin-4-yl)benzyl)-1H-1,2,3-triazole-4-carboxamide (48 mg, 43% yield) as a white solid. LCMS m/z=516.3 (M+H)+. ¹H NMR (500 MHz, DMSO-d₆) δ: 8.97 (s, 1H), 8.71 (s, 1H), 8.34 (s, 1H), 8.25 (s, 1H), 7.55-7.14 (m, 4H), 6.77-5.42 (m, 3H), 4.54-4.41 (m, 2H), 3.74-3.36 (m, 1H), 3.19-2.64 (m, 7H), 2.38 (s, 3H), 1.72-1.51 (m, 13H).

### Example 2: 1-(tert-butyl)-N-(2-methyl-4-(3-(3-(N-methylacrylamido)pyrrolidin-1-yl)pyridin-4-yl)benzyl)-1H-1,2,3-triazole-4-carboxamide

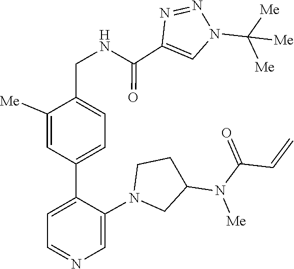 [SMILES: C=CC(=O)N(C)C1CCN(c2cnccc2-c2ccc(CNC(=O)c3cn(C(C)(C)C)nn3)c(C)c2)C1]

### 1. Synthesis of tert-butyl (1-(4-chloropyridin-3-yl)pyrrolidin-3-yl)(methyl)carbamate

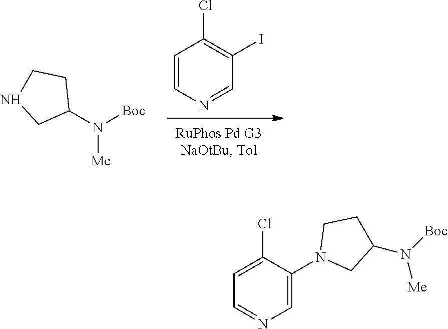 [SMILES: CN(C(=O)OC(C)(C)C)C1CCNC1 ; Clc1ccncc1I ; CN(C(=O)OC(C)(C)C)C1CCN(c2cnccc2Cl)C1]

tert-Butyl (1-(4-chloropyridin-3-yl)pyrrolidin-3-yl)(methyl)carbamate was obtained as a yellow solid, from 4-chloro-3-iodopyridine and tert-butyl methyl(pyrrolidin-3-yl)carbamate following the procedure described in Example 1, step 1: tert-butyl (1-(4-chloropyridin-3-yl)piperidin-3-yl)(methyl)carbamate (200 mg, 65% yield). LCMS m/z=312.2 (M+H)+.

### 2. Synthesis of tert-butyl (1-(4-(4-((1-(tert-butyl)-1H-1,2,3-triazole-4-carboxamido)methyl)-3-methylphenyl)pyridin-3-yl)pyrrolidin-3-yl)(methyl)carbamate

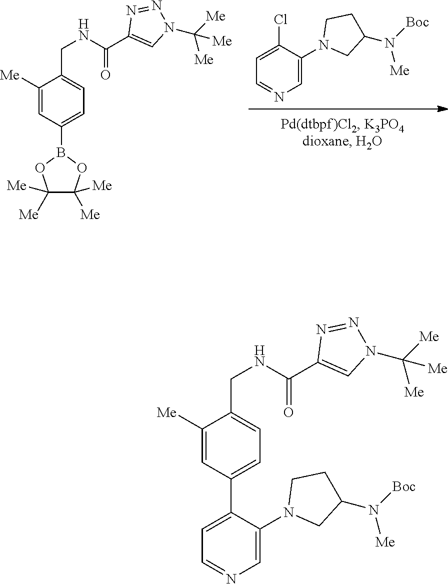 [SMILES: Cc1cc(B2OC(C)(C)C(C)(C)O2)ccc1CNC(=O)c1cn(C(C)(C)C)nn1 ; CN(C(=O)OC(C)(C)C)C1CCN(c2cnccc2Cl)C1 ; Cc1cc(-c2ccncc2N2CCC(N(C)C(=O)OC(C)(C)C)C2)ccc1CNC(=O)c1cn(C(C)(C)C)nn1]

To a solution of Intermediate 1: 1-(tert-butyl)-N-(2-methyl-4-(4,4,5,5-tetramethyl-1,3,2-dioxaborolan-2-yl)benzyl)-1H-1,2,3-triazole-4-carboxamide (200 mg, 502 μmol) in dioxane (20 mL) and water (2 mL) was added tert-butyl (1-(4-chloropyridin-3-yl)pyrrolidin-3-yl)(methyl)carbamate (172 mg, 552 μmol) and K₃PO₄ (320 mg, 1.51 mmol) at 20° C. Pd(dtbpf)Cl₂ (65 mg, 100 μmol) was then added and the mixture stirred at 90° C. for 5 h under N₂. The cooled mixture was filtered and concentrated in vacuo. The crude was purified by column chromatography on silica gel eluting with PE/EtOAc (100/0 to 0/100) to give tert-butyl (1-(4-(4-((1-(tert-butyl)-1H-1,2,3-triazole-4-carboxamido)methyl)-3-methylphenyl)pyridin-3-yl)pyrrolidin-3-yl)(methyl)carbamate (160 mg, 51% yield) as a yellow solid. LCMS m/z=548.4 (M+H)+.

### 3. Synthesis of 1-(tert-butyl)-N-(2-methyl-4-(3-(3-(methylamino)pyrrolidin-1-yl)pyridin-4-yl)benzyl)-1H-1,2,3-triazole-4-carboxamide hydrochloride

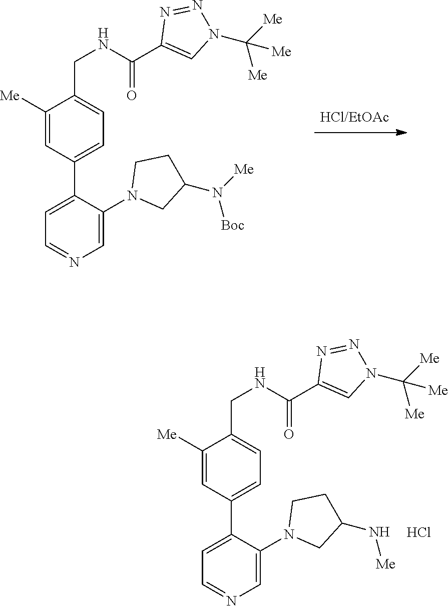 [SMILES: Cc1cc(-c2ccncc2N2CCC(N(C)C(=O)OC(C)(C)C)C2)ccc1CNC(=O)c1cn(C(C)(C)C)nn1 ; CNC1CCN(c2cnccc2-c2ccc(CNC(=O)c3cn(C(C)(C)C)nn3)c(C)c2)C1 ; Cl]

A solution of tert-butyl (1-(4-(4-((1-(tert-butyl)-1H-1,2,3-triazole-4-carboxamido)methyl)-3-methylphenyl)pyridin-3-yl)pyrrolidin-3-yl)(methyl)carbamate (160 mg, 257 μmol) in 4M HCl/EtOAc (20 mL) was stirred at 20° C. for 1 h. The mixture was evaporated under reduced pressure to give 1-(tert-butyl)-N-(2-methyl-4-(3-(3-(methylamino)pyrrolidin-1-yl)pyridin-4-yl)benzyl)-1H-1,2,3-triazole-4-carboxamide hydrochloride (130 mg, crude), as a white solid, which was used without further purification. LCMS m/z=448.3 (M+H)+.

### 4. Synthesis of 1-(tert-butyl)-N-(2-methyl-4-(3-(3-(N-methylacrylamido)pyrrolidin-1-yl)pyridin-4-yl)benzyl)-1H-1, 2,3-triazole-4-carboxamide

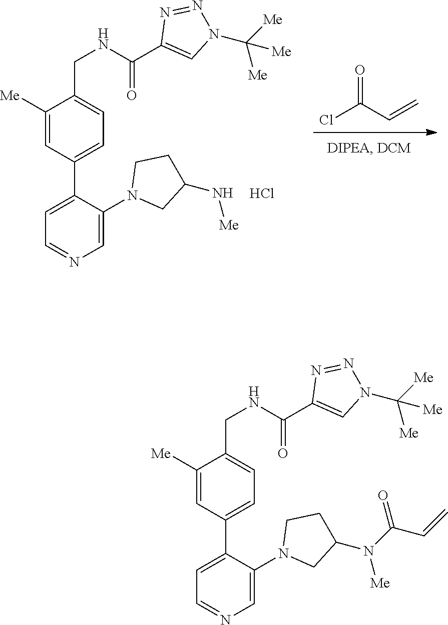

To a solution of 1-(tert-butyl)-N-(2-methyl-4-(3-(3-(methylamino)pyrrolidin-1-yl)pyridin-4-yl)benzyl)-1H-1,2,3-triazole-4-carboxamide hydrochloride (120 mg, 248 μmol) in DCM (30 mL) was added DIPEA (64 mg, 496 μmol) at 20° C. Acryloyl chloride (27 mg, 298 μmol) was added slowly and the reaction was stirred at 20° C. for 1 h. The mixture was poured into water (50 mL) and extracted with DCM (50 mL×3). The combined organic layers were washed with brine (100 mL), dried over Na₂SO₄, filtered, and concentrated in vacuo. The crude product was purified by prep-HPLC (Method A2, organic gradient 30-60%) to give 1-(tert-butyl)-N-(2-methyl-4-(3-(3-(N-methylacrylamido)pyrrolidin-1-yl)pyridin-4-yl)benzyl)-1H-1,2,3-triazole-4-carboxamide (44 mg, 35% yield) as a yellow solid. LCMS m/z=502.3 (M+H)+. ¹H NMR (400 MHz, DMSO-d₆) δ: 8.95 (s, 1H), 8.68 (s, 1H), 8.19 (s, 1H), 8.08 (d, 1H), 7.30-7.21 (m, 3H), 7.05 (d, 1H), 6.76-6.10 (m, 1H), 6.08-5.97 (m, 1H), 5.62 (s, 1H), 5.07-4.65 (m, 1H), 4.46 (d, 2H), 3.09-2.67 (m, 7H), 2.34 (s, 3H), 1.98-1.77 (m, 2H), 1.62 (s, 9H).

### Example 3: N-(4-(3-(1-acryloyl-1,7-diazaspiro[4.4]nonan-7-yl)pyridin-4-yl)-2-methylbenzyl)-1-(tert-butyl)-1H-1,2,3-triazole-4-carboxamide

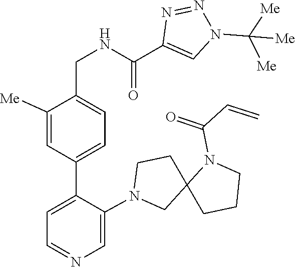 [SMILES: C=CC(=O)N1CCCC12CCN(c1cnccc1-c1ccc(CNC(=O)c3cn(C(C)(C)C)nn3)c(C)c1)C2]

### 1. Synthesis of tert-butyl 7-(4-chloropyridin-3-yl)-1,7-diazaspiro[4.4]nonane-1-carboxylate

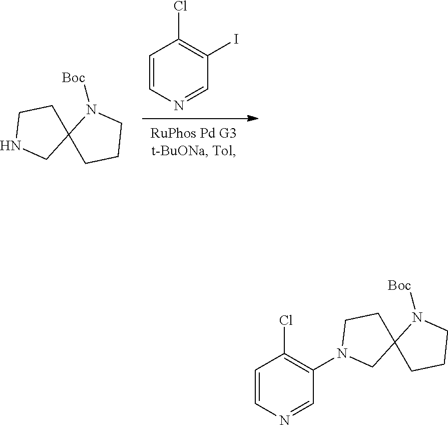

To a solution of 4-chloro-3-iodopyridine (110 mg, 460 μmol) and tert-butyl 1,7-diazaspiro[4.4]nonane-1-carboxylate (80 mg, 354 μmol) in toluene (8 mL) was added RuPhos Pd G3 (44 mg, 53 μmol) and NaOtBu (102 mg, 1.06 mmol) at 20° C. The reaction was stirred at 110° C. under N₂ for 5 h then was concentrated in vacuo. The crude product was purified by prep-TLC (PE/EtOAc=1/3) to give tert-butyl 7-(4-chloropyridin-3-yl)-1,7-diazaspiro[4.4]nonane-1-carboxylate (75 mg, 63% yield) as a pale white solid. LCMS: m/z=338.3 (M+H)+.

### 2. Synthesis of tert-butyl 7-(4-(4-((1-(tert-butyl)-1H-1,2,3-triazole-4-carboxamido)methyl)-3-methylphenyl)pyridin-3-yl)-1,7-diazaspiro[4.4]nonane-1-carboxylate

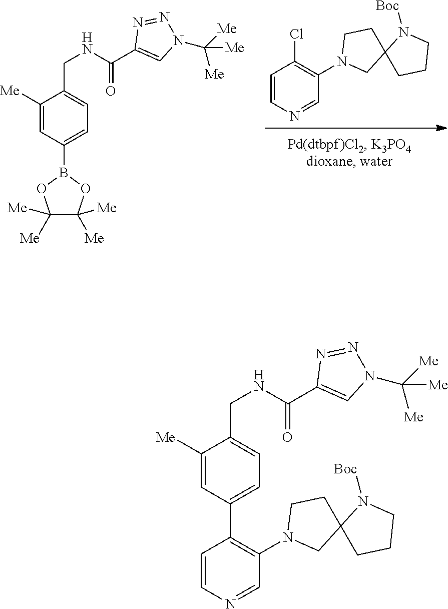 [SMILES: Cc1cc(B2OC(C)(C)C(C)(C)O2)ccc1CNC(=O)c1cn(C(C)(C)C)nn1 ; CC(C)(C)OC(=O)N1CCCC12CCN(c1cnccc1Cl)C2 ; Cc1cc(-c2ccncc2N2CCC3(CCCN3C(=O)OC(C)(C)C)C2)ccc1CNC(=O)c1cn(C(C)(C)C)nn1]

To a solution of Intermediate 1: 1-(tert-butyl)-N-(2-methyl-4-(4,4,5,5-tetramethyl-1,3,2-dioxaborolan-2-yl)benzyl)-1H-1,2,3-triazole-4-carboxamide (107 mg, 269 μmol) and tert-butyl 7-(4-chloropyridin-3-yl)-1,7-diazaspiro[4.4]nonane-1-carboxylate (70 mg, 207 μmol) in a mixture of dioxane (4 mL) and water (1 mL) was added K₃PO₄ (88 mg, 414 μmol) and Pd(dtbpf)Cl₂ (20 mg, 31 μmol) at 20° C. The reaction was stirred at 90° C. under N₂ for 5 h, then was allowed to cool and concentrated in vacuo. The crude product was purified by prep-TLC (EtOAc) to give tert-butyl 7-(4-(4-((1-(tert-butyl)-1H-1,2,3-triazole-4-carboxamido)methyl)-3-methylphenyl)pyridin-3-yl)-1,7-diazaspiro[4.4]nonane-1-carboxylate (75 mg, 63% yield) as a light gray solid. LCMS m/z=574.4 (M+H)+.

### 3. Synthesis of N-(4-(3-(1,7-diazaspiro[4.4]nonan-7-yl)pyridin-4-yl)-2-methylbenzyl)-1-(tert-butyl)-1H-1,2,3-triazole-4-carboxamide trifluoroacetate

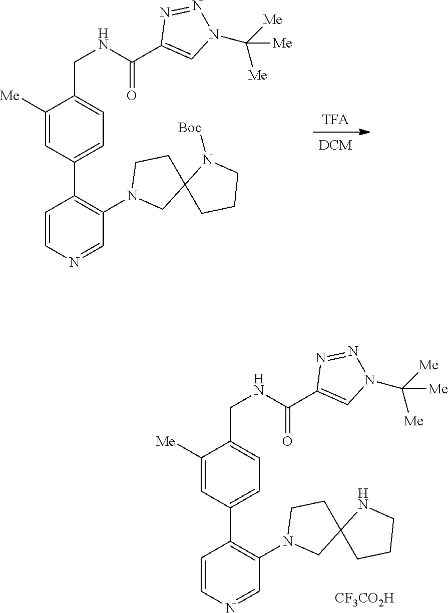

To a solution of tert-butyl 7-(4-(4-((1-(tert-butyl)-1H-1,2,3-triazole-4-carboxamido)methyl)-3-methylphenyl)pyridin-3-yl)-1,7-diazaspiro[4.4]nonane-1-carboxylate (75 mg, 131 μmol) in DCM (20 mL) was added TFA (4 mL) and the reaction stirred at 20° C. for 1 h. The reaction mixture was evaporated under reduced pressure to give N-(4-(3-(1,7-diazaspiro[4.4]nonan-7-yl)pyridin-4-yl)-2-methylbenzyl)-1-(tert-butyl)-1H-1,2,3-triazole-4-carboxamide trifluoroacetate (62 mg, crude) as light brown oil. LCMS m/z=474.3 (M+H)+.

### 4. Synthesis of N-(4-(3-(1-acryloyl-1,7-diazaspiro[4.4]nonan-7-yl)pyridin-4-yl)-2-methylbenzyl)-1-(tert-butyl)-1H-1,2,3-triazole-4-carboxamide

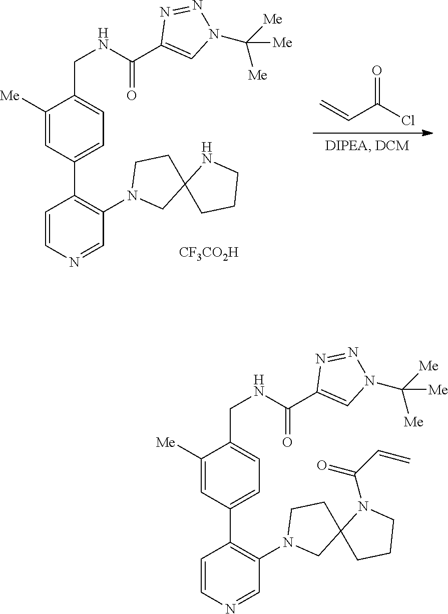

To a solution of N-(4-(3-(1,7-diazaspiro[4.4]nonan-7-yl)pyridin-4-yl)-2-methylbenzyl)-1-(tert-butyl)-1H-1,2,3-triazole-4-carboxamide trifluoroacetate (62 mg, 131 μmol) and DIPEA (68 mg, 524 μmol) in DCM (35 mL) was added acryloyl chloride (15 mg, 170 μmol) and the reaction was stirred at 20° C. for 1 h. The reaction was quenched with MeOH (2 mL) and the mixture was concentrated in vacuo. The crude product was purified by prep-HPLC (Method A2, organic gradient 34-64%) to give N-(4-(3-(1-acryloyl-1,7-diazaspiro[4.4]nonan-7-yl)pyridin-4-yl)-2-methylbenzyl)-1-(tert-butyl)-1H-1,2,3-triazole-4-carboxamide (38 mg, 50% yield) as a pale white solid. LCMS m/z=528.3 (M+H)+. ¹H NMR (400 MHz, DMSO-d₆) δ: 8.92 (t, 1H), 8.67 (s, 1H), 8.12-8.03 (m, 2H), 7.28-7.20 (m, 3H), 7.03 (d, 1H), 6.51-6.44 (m, 1H), 6.06-6.00 (m, 1H), 5.59-5.55 (m, 1H), 4.43 (d, 2H), 3.49-3.43 (m, 2H), 3.06-3.01 (m, 2H), 2.64-2.55 (m, 2H), 2.33 (s, 3H), 1.83-1.48 (m, 15H).

### Example 4: N-(4-(3-(7-acryloyl-2,7-diazaspiro[4.4]nonan-2-yl)pyridin-4-yl)-2-methylbenzyl)-1-(tert-butyl)-1H-1,2,3-triazole-4-carboxamide

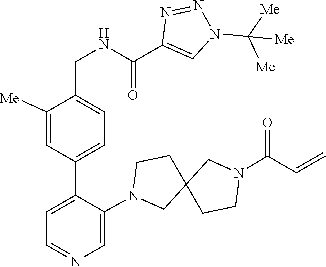 [SMILES: C=CC(=O)N1CCC2(CCN(c3cnccc3-c3ccc(CNC(=O)c4cn(C(C)(C)C)nn4)c(C)c3)C2)C1]

### 1. Synthesis of tert-butyl 7-(4-chloropyridin-3-yl)-2,7-diazaspiro[4.4]nonane-2-carboxylate

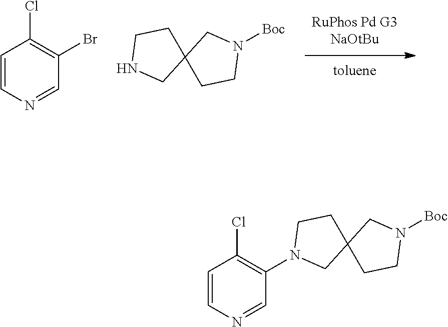 [SMILES: Clc1ccncc1Br ; CC(C)(C)OC(=O)N1CCC2(CCNC2)C1 ; CC(C)(C)OC(=O)N1CCC2(CCN(c3cnccc3Cl)C2)C1]

A mixture of 4-chloro-3-iodopyridine (160 mg, 668 μmol), tert-butyl 2,7-diazaspiro[4.4]nonane-2-carboxylate (151 mg, 668 μmol), RuPhos Pd G3 (56 mg, 67 μmol), and NaOtBu (193 mg, 2.0 mmol) in toluene (4 mL) was purged with N₂ for 5 mins, then was heated at 100° C. for 2 h. The cooled mixture was concentrated in vacuo, the residue was suspended in EtOAc and washed with H₂O (2×) and brine, then concentrated in vacuo. The crude was purified by silica gel column chromatography eluting with EtOAC/heptanes (0/100 to 60/40) to give tert-butyl 7-(4-chloropyridin-3-yl)-2,7-diazaspiro[4.4]nonane-2-carboxylate (170 mg, 75% yield). LCMS m/z=338.1 (M+H)+.

### 2. Synthesis of tert-butyl 7-(4-(4-((1-(tert-butyl)-1H-1,2,3-triazole-4-carboxamido)methyl)-3-methylphenyl)pyridin-3-yl)-2,7-diazaspiro[4.4]nonane-2-carboxylate

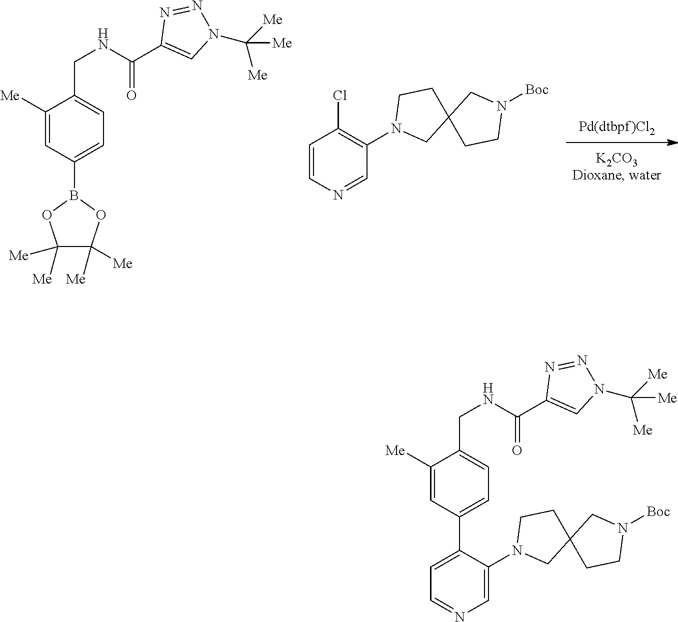 [SMILES: Cc1cc(B2OC(C)(C)C(C)(C)O2)ccc1CNC(=O)c1cn(C(C)(C)C)nn1 ; CC(C)(C)OC(=O)N1CCC2(CCN(c3cnccc3Cl)C2)C1 ; Cc1cc(-c2ccncc2N2CCC3(CCN(C(=O)OC(C)(C)C)C3)C2)ccc1CNC(=O)c1cn(C(C)(C)C)nn1]

To a solution of Intermediate 1: 1-(tert-butyl)-N-(2-methyl-4-(4,4,5,5-tetramethyl-1,3,2-dioxaborolan-2-yl)benzyl)-1H-1,2,3-triazole-4-carboxamide (236 mg, 592 μmol) in dioxane (4.2 mL) and water (700 μL) was added tert-butyl 7-(4-chloropyridin-3-yl)-2,7-diazaspiro[4.4]nonane-2-carboxylate (200 mg, 592 μmol), K₂CO₃ (246 mg, 1.78 mmol) and Pd(dtbpf)Cl₂ (39 mg, 59 μmol) and the mixture was purged with N₂ for 10 mins then sealed. The reaction was stirred at 90° C. under N₂ for 5 h, allowed to cool, and concentrated in vacuo. The residue was taken up in DCM, filtered, and purified by silica gel column chromatography eluting with EtOAc/heptanes (0/100 to 100/0) to give tert-butyl 7-(4-(4-((1-(tert-butyl)-1H-1,2,3-triazole-4-carboxamido)methyl)-3-methylphenyl)pyridin-3-yl)-2,7-diazaspiro[4.4]nonane-2-carboxylate (100 mg, 29% yield). LCMS m/z=574.4 (M+H)+.

### 3. Synthesis of N-(4-(3-(2,7-diazaspiro[4.4]nonan-2-yl)pyridin-4-yl)-2-methylbenzyl)-1-(tert-butyl)-1,2,3-triazole-4-carboxamide hydrochloride

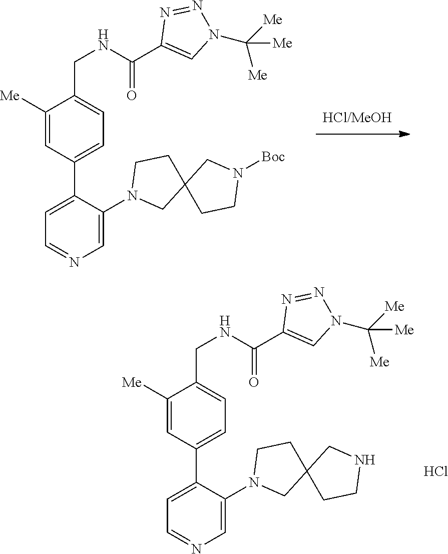 [SMILES: Cc1cc(-c2ccncc2N2CCC3(CCN(C(=O)OC(C)(C)C)C3)C2)ccc1CNC(=O)c1cn(C(C)(C)C)nn1 ; Cc1cc(-c2ccncc2N2CCC3(CCNC3)C2)ccc1CNC(=O)c1cn(C(C)(C)C)nn1 ; Cl]

A solution of tert-butyl 7-(4-(4-((1-(tert-butyl)-1H-1,2,3-triazole-4-carboxamido)methyl)-3-methylphenyl)pyridin-3-yl)-2,7-diazaspiro[4.4]nonane-2-carboxylate (100 mg, 175 μmol) in HCl (4 M, 437 μL) and MeOH (2 drops) was stirred for 2 h at r. The reaction was evaporated under reduced pressure to give N-(4-(3-(2,7-diazaspiro[4.4]nonan-2-yl)pyridin-4-yl)-2-methylbenzyl)-1-(tert-butyl)-1H-1,2,3-triazole-4-carboxamide hydrochloride (80 mg, crude) which was carried forward without further purification. LCMS m/z=474.3 (M+H)+.

### 4. Synthesis of N-(4-(3-(7-acryloyl-2,7-diazaspiro[4.4]nonan-2-yl)pyridin-4-yl)-2-methylbenzyl)-1-(tert-butyl)-1H-1,2,3-triazole-4-carboxamide

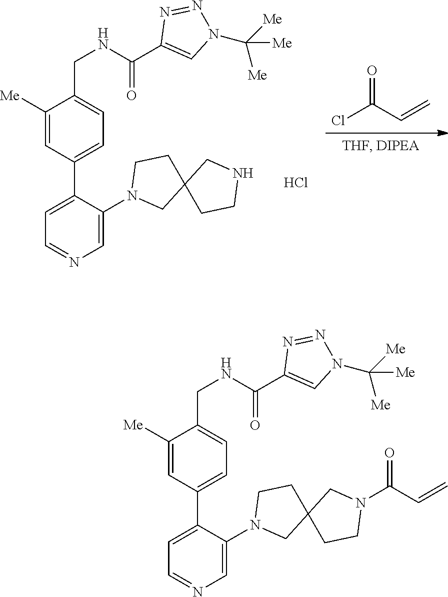

To a solution of N-(4-(3-(2,7-diazaspiro[4.4]nonan-2-yl)pyridin-4-yl)-2-methylbenzyl)-1-(tert-butyl)-1H-1,2,3-triazole-4-carboxamide hydrochloride (80 mg, 169 μmol) in THF (2 mL) was added DIPEA (109 mg, 845 μmol) and acryloyl chloride (31 mg, 338 μmol) and the reaction was stirred 25° C. for 30 mins. The mixture was concentrated in vacuo and the residue was purified by silica gel column chromatography eluting with EtOAc/EtOH (100/0 to 75/25) to give the product N-(4-(3-(7-acryloyl-2,7-diazaspiro[4.4]nonan-2-yl)pyridin-4-yl)-2-methylbenzyl)-1-(tert-butyl)-1H-1,2,3-triazole-4-carboxamide (61 mg, 68% yield). LCMS m/z=528.4 (M+H)+. ¹H NMR (400 MHz, CDCl₃) δ: 8.24-8.17 (m, 2H), 8.14 (dd, 1H), 7.48 (br s, 1H), 7.36 (d, 1H), 7.27-7.21 (m, 2H), 7.06 (dd, 1H), 6.51-6.33 (m, 2H), 5.75-5.64 (m, 1H), 4.70 (d, 2H), 3.69-3.40 (m, 4H), 3.00 (s, 4H), 2.42 (d, 3H), 2.01-1.76 (m, 4H), 1.72 (s, 9H).

### Example 5: N-(4-(3-(7-acryloyl-2,7-diazaspiro[4.4]nonan-2-yl)pyridin-4-yl)-2-methylbenzyl)-3-(difluoromethyl)-1-methyl-1H-pyrazole-4-carboxamide

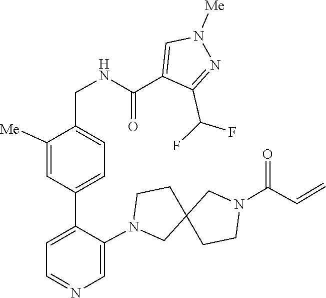 [SMILES: C=CC(=O)N1CCC2(CCN(c3cnccc3-c3ccc(CNC(=O)c4cn(C)nc4C(F)F)c(C)c3)C2)C1]

N-(4-(3-(7-acryloyl-2,7-diazaspiro[4.4]nonan-2-yl)pyridin-4-yl)-2-methylbenzyl)-3-(difluoromethyl)-1-methyl-1H-pyrazole-4-carboxamide was obtained from Example 4, step 1: tert-butyl 7-(4-chloropyridin-3-yl)-2,7-diazaspiro[4.4]nonane-2-carboxylate and Intermediate 6: 3-(difluoromethyl)-1-methyl-N-(2-methyl-4-(4,4,5,5-tetramethyl-1,3,2-dioxaborolan-2-yl)benzyl)-1H-pyrazole-4-carboxamide, following the steps described in Example 4 (61 mg, 63% yield). LCMS m/z=535.3 (M+H)+. ¹H NMR (400 MHz, CDCl₃) δ: 8.19-8.06 (m, 3H), 7.36-7.22 (m, 1H), 7.19-7.07 (m, 2H), 7.04-6.86 (m, 2H), 6.34-6.14 (m, 2H), 5.69-5.56 (m, 1H), 4.82-4.67 (m, 1H), 4.38-4.25 (m, 1H), 3.87 (s, 3H), 3.55-3.37 (m, 2H), 3.36-3.21 (m, 1H), 3.20-2.97 (m, 3H), 2.82-2.70 (m, 1H), 2.58-2.46 (m, 1H), 2.30 (d, 3H), 1.68-1.87 (m, 4H).

### Example 6: N-(4-(3-(6-acryloyl-2,6-diazaspiro[3.3]heptan-2-yl)pyridin-4-yl)-2-methylbenzyl)-5-(tert-butyl)-1,2,4-oxadiazole-3-carboxamide

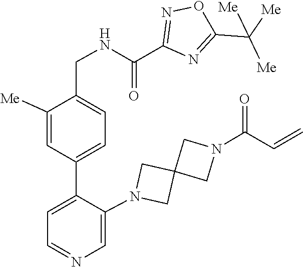 [SMILES: C=CC(=O)N1CC2(C1)CN(c1cnccc1-c1ccc(CNC(=O)c3noc(C(C)(C)C)n3)c(C)c1)C2]

### 1. Synthesis of tert-butyl 6-(4-chloropyridin-3-yl)-2,6-diazaspiro[3.3]heptane-2-carboxylate

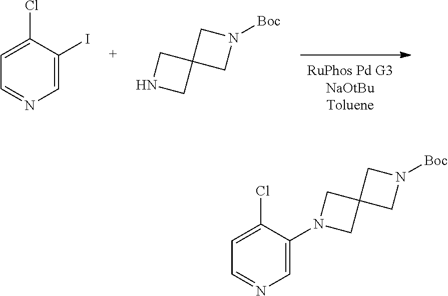 [SMILES: Clc1ccncc1I ; CC(C)(C)OC(=O)N1CC2(CNC2)C1 ; CC(C)(C)OC(=O)N1CC2(C1)CN(c1cnccc1Cl)C2]

tert-Butyl 6-(4-chloropyridin-3-yl)-2,6-diazaspiro[3.3]heptane-2-carboxylate was obtained as a pale oil from 4-chloro-3-iodopyridine and tert-butyl 2,6-diazaspiro[3.3]heptane-2-carboxylate, following the procedure described in Example 4, step 1: tert-butyl 7-(4-chloropyridin-3-yl)-2,7-diazaspiro[4.4]nonane-2-carboxylate. LCMS m/z=310.0 (M+H)+.

### 2. Synthesis of tert-butyl 6-(4-(4-((5-(tert-butyl)-1,2,4-oxadiazole-3-carboxamido)methyl)-3-methylphenyl)pyridin-3-yl)-2,6-diazaspiro[3.3]heptane-2-carboxylate

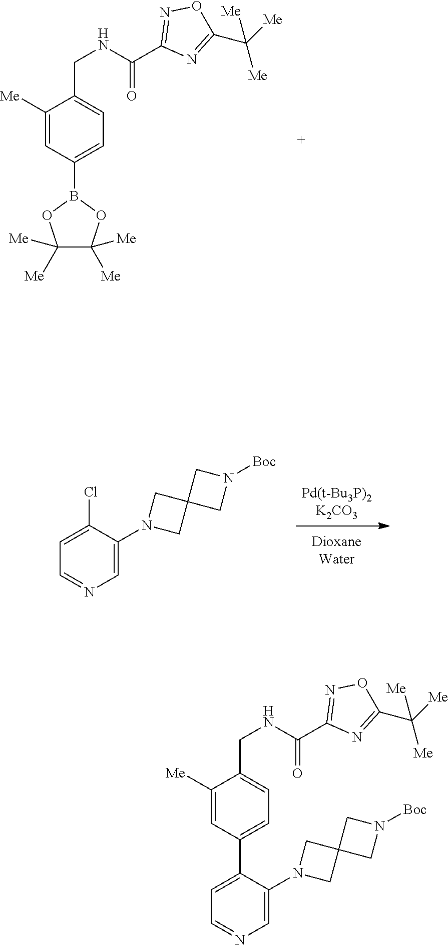 [SMILES: Cc1cc(B2OC(C)(C)C(C)(C)O2)ccc1CNC(=O)c1noc(C(C)(C)C)n1 ; CC(C)(C)OC(=O)N1CC2(C1)CN(c1cnccc1Cl)C2 ; Cc1cc(-c2ccncc2N2CC3(CN(C(=O)OC(C)(C)C)C3)C2)ccc1CNC(=O)c1noc(C(C)(C)C)n1]

A mixture of tert-butyl 6-(4-chloropyridin-3-yl)-2,6-diazaspiro[3.3]heptane-2-carboxylate (250 mg, 807 μmol), Intermediate 5: 5-(tert-butyl)-N-(2-methyl-4-(4,4,5,5-tetramethyl-1,3,2-dioxaborolan-2-yl)benzyl)-1,2,4-oxadiazole-3-carboxamide (483 mg, 1.21 mmol), K₂CO₃ (446 mg, 3.23 mmol) and Pd(t-Bu3P)₂ (41 mg, 81 μmol) in dioxane (6 mL) and water (1 mL) was purged with N₂ for 5 mins. The reaction was heated to 100° C. and stirred for 2 h. The cooled reaction was concentrated in vacuo and the residue was purified by silica gel column chromatography eluting with (3/1 EtOAc:EtOH):Heptane (0/100 to 75/25) to give tert-butyl 6-(4-(4-((5-(tert-butyl)-1,2,4-oxadiazole-3-carboxamido)methyl)-3-methylphenyl)pyridin-3-yl)-2,6-diazaspiro[3.3]heptane-2-carboxylate (299 mg, 68% yield) as an off-white solid. LCMS m/z=547.2 (M+H)+.

### 3. Synthesis of N-(4-(3-(2,6-diazaspiro[3.3]heptan-2-yl)pyridin-4-yl)-2-methylbenzyl)-5-(tert-butyl)-1,2,4-oxadiazole-3-carboxamide hydrochloride

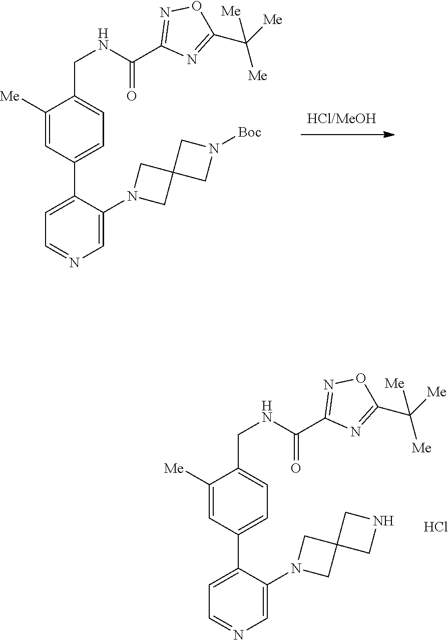 [SMILES: Cc1cc(-c2ccncc2N2CC3(CN(C(=O)OC(C)(C)C)C3)C2)ccc1CNC(=O)c1noc(C(C)(C)C)n1 ; Cc1cc(-c2ccncc2N2CC3(CNC3)C2)ccc1CNC(=O)c1noc(C(C)(C)C)n1 ; Cl]

A solution of tert-butyl 6-(4-(4-((5-(tert-butyl)-1,2,4-oxadiazole-3-carboxamido)methyl)-3-methylphenyl)pyridin-3-yl)-2,6-diazaspiro[3.3]heptane-2-carboxylate (299 mg, 547 μmol) in MeOH (1.8 mL) and HCl/MeOH (1.25 M, 4.4 mL) was stirred at 50° C. for 6 h. The solution was evaporated under reduced pressure to give N-(4-(3-(2,6-diazaspiro[3.3]heptan-2-yl)pyridin-4-yl)-2-methylbenzyl)-5-(tert-butyl)-1,2,4-oxadiazole-3-carboxamide hydrochloride as a yellow solid (304 mg, 23% yield). LCMS m/z=447.1 (M+H)+.

### 4. Synthesis of N-(4-(3-(6-acryloyl-2,6-diazaspiro[3.3]heptan-2-yl)pyridin-4-yl)-2-methylbenzyl)-5-(tert-butyl)-1,2,4-oxadiazole-3-carboxamide

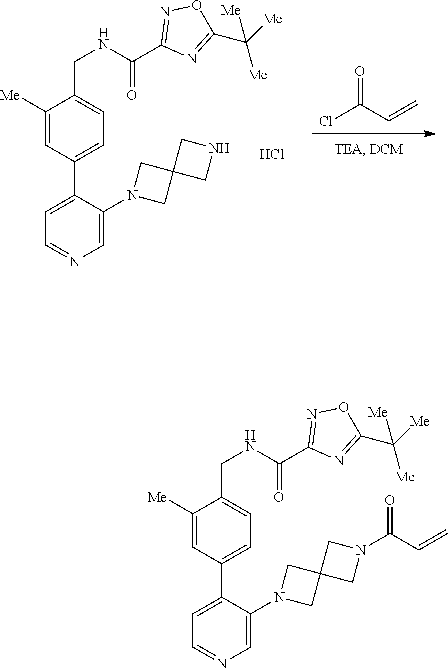

DCM (4.8 mL) and TEA (289 mg, 2.86 mmol) were added to N-(4-(3-(2,6-diazaspiro[3.3]heptan-2-yl)pyridin-4-yl)-2-methylbenzyl)-5-(tert-butyl)-1,2,4-oxadiazole-3-carboxamide hydrochloride (69 mg, 143 μmol) and the mixture was stirred for 5 mins. The solution was cooled in a dry ice/acetone bath, acryloyl chloride (65 mg, 715 μmol) was added dropwise, and the reaction was stirred for 15 mins. The reaction mixture was purified directly by silica gel column chromatography (3:1 EtOAc:EtOH):heptanes (0/100 to 100/0) to give N-(4-(3-(6-acryloyl-2,6-diazaspiro[3.3]heptan-2-yl)pyridin-4-yl)-2-methylbenzyl)-5-(tert-butyl)-1,2,4-oxadiazole-3-carboxamide (29 mg, 40% yield) as a light yellow film. LCMS m/z=501.2 (M+H)+. ¹H NMR (500 MHz, MeOH-d₄) δ: 8.00 (d, 1H), 7.85 (s, 1H), 7.39 (d, 1H), 7.30-7.24 (m, 2H), 7.11 (d, 1H), 6.31-6.15 (m, 2H), 5.73-5.66 (m, 1H), 4.64 (s, 2H), 4.33 (s, 2H), 4.08 (s, 2H), 3.73 (s, 4H), 2.44 (s, 3H), 1.48 (s, 9H).

### Example 7: N-(4-(3-(2-acryloyl-2,6-diazaspiro[3.4]octan-6-yl)pyridin-4-yl)-2-methylbenzyl)-5-(tert-butyl)-1,2,4-oxadiazole-3-carboxamide

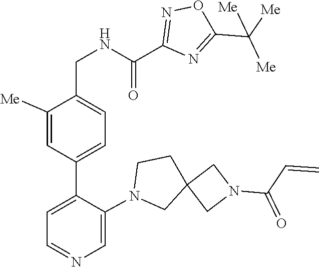 [SMILES: C=CC(=O)N1CC2(CCN(c3cnccc3-c3ccc(CNC(=O)c4noc(C(C)(C)C)n4)c(C)c3)C2)C1]

### 1. Synthesis of tert-butyl 6-(4-chloropyridin-3-yl)-2,6-diazaspiro[3.4]octane-2-carboxylate

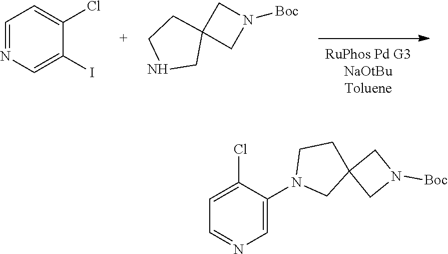 [SMILES: Clc1ccncc1I ; CC(C)(C)OC(=O)N1CC2(CCNC2)C1 ; CC(C)(C)OC(=O)N1CC2(CCN(c3cnccc3Cl)C2)C1]

tert-Butyl 6-(4-chloropyridin-3-yl)-2,6-diazaspiro[3.4]octane-2-carboxylate was obtained as a pale oil from 4-chloro-3-iodopyridine and tert-butyl 2,6-diazaspiro[3.4]octane-2-carboxylate, following the procedure described in Example 4, step 1: tert-butyl 7-(4-chloropyridin-3-yl)-2,7-diazaspiro[4.4]nonane-2-carboxylate (333 mg, 49% yield). LCMS m/z=324.0 (M+H)+.

### 2. Synthesis of tert-butyl 6-(4-(4-((5-(tert-butyl)-1,2,4-oxadiazole-3-carboxamido)methyl)-3-methylphenyl)pyridin-3-yl)-2,6-diazaspiro[3.4]octane-2-carboxylate

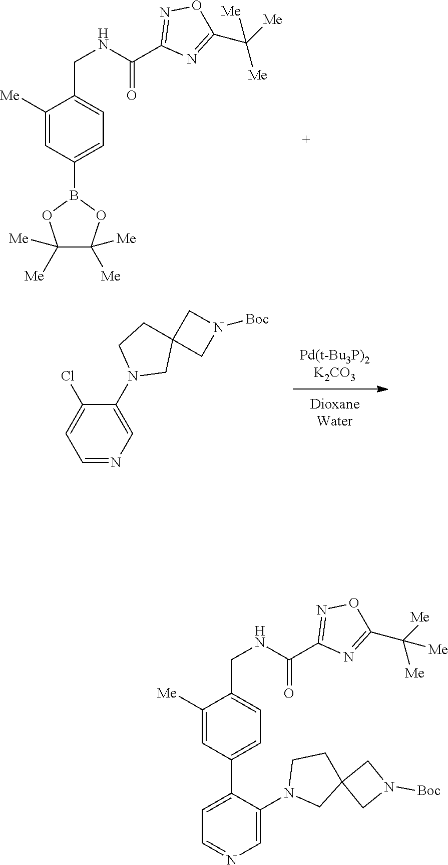 [SMILES: Cc1cc(B2OC(C)(C)C(C)(C)O2)ccc1CNC(=O)c1noc(C(C)(C)C)n1 ; CC(C)(C)OC(=O)N1CC2(CCN(c3cnccc3Cl)C2)C1 ; Cc1cc(-c2ccncc2N2CCC3(CN(C(=O)OC(C)(C)C)C3)C2)ccc1CNC(=O)c1noc(C(C)(C)C)n1]

tert-Butyl 6-(4-(4-((5-(tert-butyl)-1,2,4-oxadiazole-3-carboxamido)methyl)-3-methylphenyl)pyridin-3-yl)-2,6-diazaspiro[3.4]octane-2-carboxylate was obtained from tert-butyl 6-(4-chloropyridin-3-yl)-2,6-diazaspiro[3.4]octane-2-carboxylate and Intermediate 5: 5-(tert-butyl)-N-(2-methyl-4-(4,4,5,5-tetramethyl-1,3,2-dioxaborolan-2-yl)benzyl)-1,2,4-oxadiazole-3-carboxamide following the procedure described in Example 6, step 2: N-(4-(3-(6-acryloyl-2,6-diazaspiro[3.3]heptan-2-yl)pyridin-4-yl)-2-methylbenzyl)-5-(tert-butyl)-1,2,4-oxadiazole-3-carboxamide. LCMS m/z=561.3 (M+H)+.

### 3. Synthesis of N-(4-(3-(2,6-diazaspiro[3.4]octan-6-yl)pyridin-4-yl)-2-methylbenzyl)-5-(tert-butyl)-1,2,4-oxadiazole-3-carboxamide

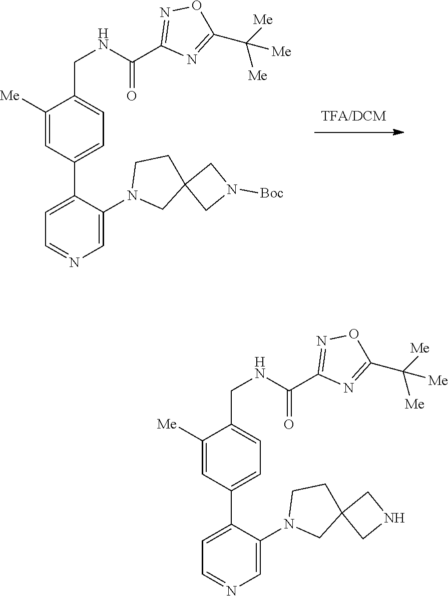 [SMILES: Cc1cc(-c2ccncc2N2CCC3(CN(C(=O)OC(C)(C)C)C3)C2)ccc1CNC(=O)c1noc(C(C)(C)C)n1 ; Cc1cc(-c2ccncc2N2CCC3(CNC3)C2)ccc1CNC(=O)c1noc(C(C)(C)C)n1]

TFA (1.0 g, 8.85 mmol) was added to a solution of tert-butyl 6-(4-(4-((5-(tert-butyl)-1,2,4-oxadiazole-3-carboxamido)methyl)-3-methylphenyl)pyridin-3-yl)-2,6-diazaspiro[3.4]octane-2-carboxylate (496 mg, 885 μmol) in DCM (4.4 mL) and the reaction was stirred for 18 h at rt. The material was passed through an SCX ion exchange column and washed with MeOH. The product was liberated by eluting with 2M NH₃/MeOH, and the filtrate was evaporated to give N-(4-(3-(2,6-diazaspiro[3.4]octan-6-yl)pyridin-4-yl)-2-methylbenzyl)-5-(tert-butyl)-1,2,4-oxadiazole-3-carboxamide (368 mg, 90% yield) as a light yellow oily solid. LCMS m/z=461.3 (M+H)+.

### 4. Synthesis of N-(4-(3-(2-acryloyl-2,6-diazaspiro[3.4]octan-6-yl)pyridin-4-yl)-2-methylbenzyl)-5-(tert-butyl)-1,2,4-oxadiazole-3-carboxamide

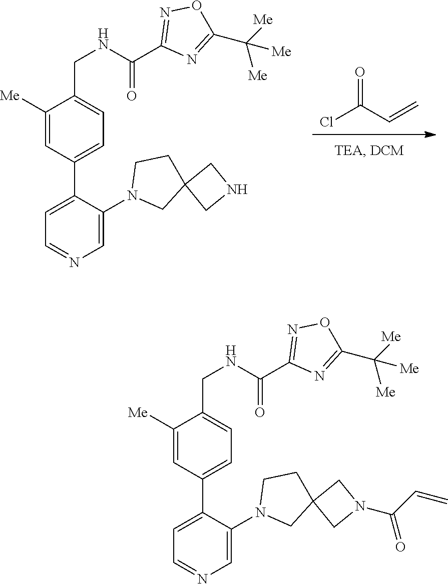

N-(4-(3-(2-acryloyl-2,6-diazaspiro[3.4]octan-6-yl)pyridin-4-yl)-2-methylbenzyl)-5-(tert-butyl)-1,2,4-oxadiazole-3-carboxamide was obtained as a light yellow film from N-(4-(3-(2,6-diazaspiro[3.4]octan-6-yl)pyridin-4-yl)-2-methylbenzyl)-5-(tert-butyl)-1,2,4-oxadiazole-3-carboxamide and acryloyl chloride, following the procedure described in Example 6, step 4: N-(4-(3-(6-acryloyl-2,6-diazaspiro[3.3]heptan-2-yl)pyridin-4-yl)-2-methylbenzyl)-5-(tert-butyl)-1,2,4-oxadiazole-3-carboxamide. LCMS m/z=515.2 (M+H)+. ¹H NMR (500 MHz, DMSO-d₆) δ: 9.47-9.38 (m, 1H), 8.16 (s, 1H), 8.03 (br d, 1H), 7.34-7.20 (m, 2H), 7.05 (br d, 1H), 6.27 (br dd, 1H), 6.07 (br dd, 1H), 5.64 (br dd, 1H), 4.49 (br d, 2H), 4.08 (br s, 2H), 3.79 (s, 2H), 3.24-3.14 (m, 2H), 2.91 (br t, 2H), 2.36 (s, 3H), 1.98 (br dd, 2H), 1.43 (s, 9H).

### Example 8: 1-(tert-butyl)-N-(2-methyl-4-(5-(3-(N-methylacrylamido)piperidin-1-yl)pyrimidin-4-yl)benzyl)-1H-1,2,3-triazole-4-carboxamide

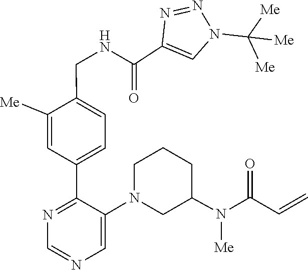 [SMILES: C=CC(=O)N(C)C1CCCN(c2cncnc2-c2ccc(CNC(=O)c3cn(C(C)(C)C)nn3)c(C)c2)C1]

### 1. Synthesis of tert-butyl (1-(4-methoxypyrimidin-5-yl)piperidin-3-yl)(methyl)carbamate

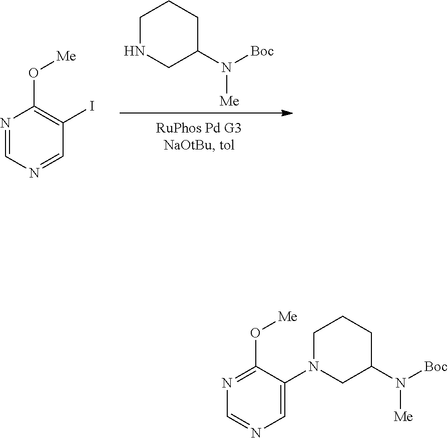 [SMILES: COc1ncncc1I ; CN(C(=O)OC(C)(C)C)C1CCCNC1 ; COc1ncncc1N1CCCC(N(C)C(=O)OC(C)(C)C)C1]

tert-Butyl (1-(4-methoxypyrimidin-5-yl)piperidin-3-yl)(methyl)carbamate was obtained as a yellow oil from 5-iodo-4-methoxypyrimidine and tert-butyl methyl(piperidin-3-yl)carbamate following the procedure described in Example 1, step 1: tert-butyl (1-(4-chloropyridin-3-yl)piperidin-3-yl)(methyl)carbamate (850 mg, 78% yield). LCMS m/z=323.5 (M+H)+.

### 2. Synthesis of 5-(3-(methylamino)piperidin-1-yl)pyrimidin-4-ol hydrobromide

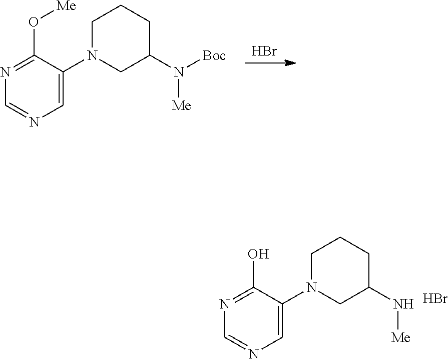 [SMILES: COc1ncncc1N1CCCC(N(C)C(=O)OC(C)(C)C)C1 ; Br ; CNC1CCCN(c2cncnc2O)C1 ; Br]

tert-Butyl (1-(4-methoxypyrimidin-5-yl)piperidin-3-yl)(methyl)carbamate (850 mg, 2.64 mmol) was added to HBr (20 mL, 40%) at 20° C. and the reaction was stirred at 80° C. for 2 h. The mixture was evaporated under reduced pressure to give 5-(3-(methylamino)-piperidin-1-yl)pyrimidin-4-ol hydrobromide (550 mg, crude) as yellow oil. LCMS m/z=209.3 (M+H)+.

### 3. Synthesis of tert-butyl (1-(4-hydroxypyrimidin-5-yl)piperidin-3-yl)(methyl)carbamate

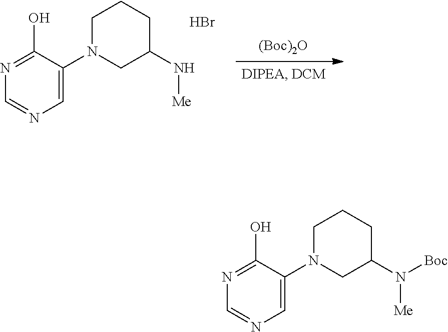 [SMILES: "(Boc)2O" ; CNC1CCCN(c2cncnc2O)C1 ; Br ; "DIPEA, DCM" ; CN(C(=O)OC(C)(C)C)C1CCCN(c2cncnc2O)C1]

To a solution of 5-(3-(methylamino)piperidin-1-yl)pyrimidin-4-ol (550 mg, 2.64 mmol) in DCM (30 mL) and MeOH (5 mL) was added DIPEA (682 mg, 5.28 mmol) and (Boc)₂₀ (1.15 g, 5.28 mmol) and the reaction was stirred at 20° C. for 5 h. The reaction was concentrated in vacuo and the residue purified by silica gel column chromatography eluting with PE to EtOAc to give tert-butyl (1-(4-hydroxypyrimidin-5-yl)piperidin-3-yl)(methyl)carbamate (500 mg, 61% yield) as yellow oil. LCMS m/z=309.5 (M+H)+.

### 4. Synthesis of tert-butyl (1-(4-(4-((1-(tert-butyl)-1H-1,2,3-triazole-4-carboxamido)methyl)-3-methylphenyl)pyrimidin-5-yl)piperidin-3-yl)(methyl)carbamate

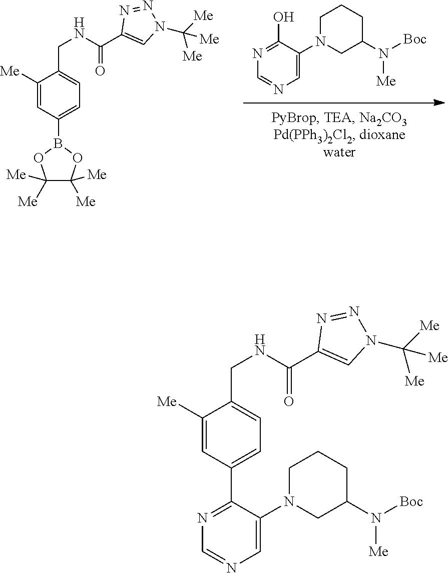 [SMILES: Cc1cc(B2OC(C)(C)C(C)(C)O2)ccc1CNC(=O)c1cn(C(C)(C)C)nn1 ; CN(C(=O)OC(C)(C)C)C1CCCN(c2cncnc2O)C1 ; Cc1cc(-c2ncncc2N2CCCC(N(C)C(=O)OC(C)(C)C)C2)ccc1CNC(=O)c1cn(C(C)(C)C)nn1]

To a solution of tert-butyl (1-(4-hydroxypyrimidin-5-yl)piperidin-3-yl)(methyl)carbamate (194 mg, 628 μmol) in dioxane (12 mL) was added TEA (191 mg, 1.88 mmol) and PyBroP (293 mg, 628 μmol) and the mixture was stirred at 20° C. for 2 h. Intermediate 1: 1-(tert-butyl)-N-(2-methyl-4-(4,4,5,5-tetramethyl-1,3,2-dioxaborolan-2-yl)benzyl)-1H-1,2,3-triazole-4-carboxamide (250 mg, 628 μmol), Na₂CO₃ (200 mg, 1.88 mmol), Pd(PPh₃)₂Cl₂ (44 mg, 63 μmol), and water (2 mL) were added and the mixture was stirred at 90° C. under N₂ for 8 h. The cooled reaction was concentrated in vacuo and the residue was purified by silica gel column chromatography eluting with PE to EtOAc to give tert-butyl (1-(4-(4-((1-(tert-butyl)-1H-1,2,3-triazole-4-carboxamido)methyl)-3-methylphenyl)pyrimidin-5-yl)piperidin-3-yl)(methyl)carbamate (170 mg, 48% yield) as yellow oil, which was carried forward without further purification.

### 5. Synthesis of 1-(tert-butyl)-N-(2-methyl-4-(5-(3-(methylamino)piperidin-1-yl)pyrimidin-4-yl)benzyl)-1H-1,2,3-triazole-4-carboxamide hydrochloride

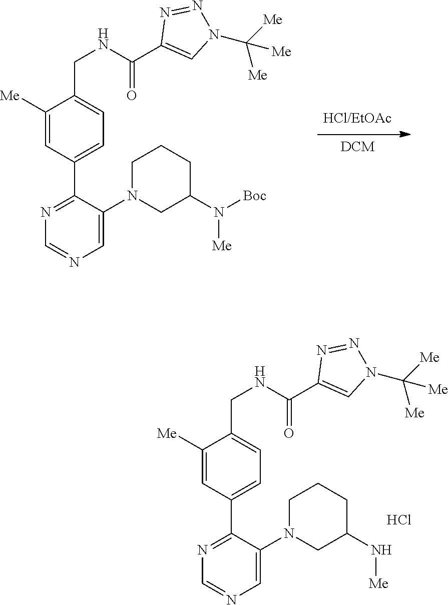

1-(tert-Butyl)-N-(2-methyl-4-(5-(3-(methylamino)piperidin-1-yl)pyrimidin-4-yl)benzyl)-1H-1,2,3-triazole-4-carboxamide hydrochloride was obtained as a white solid, from tert-butyl (1-(4-(4-((1-(tert-butyl)-1H-1,2,3-triazole-4-carboxamido)methyl)-3-methylphenyl)pyrimidin-5-yl)piperidin-3-yl)(methyl)carbamate, following the procedure described in Example 1, step 3: 1-(tert-butyl)-N-(2-methyl-4-(3-(3-(methylamino)piperidin-1-yl)pyridin-4-yl)benzyl)-1H-1,2,3-triazole-4-carboxamide hydrochloride (140 mg, crude). LCMS m/z=463.3 (M+H)+.

### 6. Synthesis of 1-(tert-butyl)-N-(2-methyl-4-(5-(3-(N-methylacrylamido)piperidin-1-yl)pyrimidin-4-yl)benzyl)-1H-1, 2,3-triazole-4-carboxamide

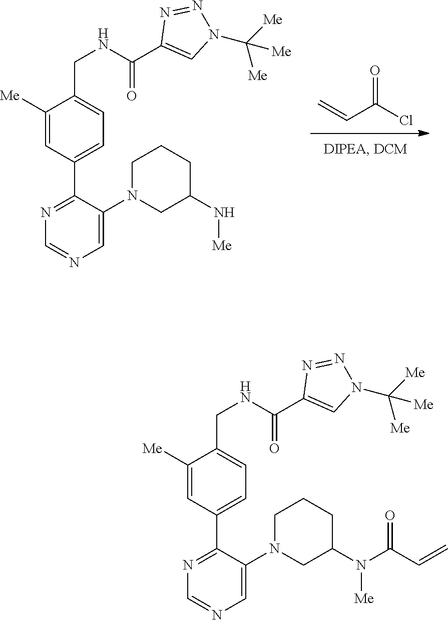

1-(tert-Butyl)-N-(2-methyl-4-(5-(3-(N-methylacrylamido)piperidin-1-yl)pyrimidin-4-yl)benzyl)-1H-1,2,3-triazole-4-carboxamide was obtained as a white solid, from 1-(tert-butyl)-N-(2-methyl-4-(5-(3-(methylamino)piperidin-1-yl)pyrimidin-4-yl)benzyl)-1H-1,2,3-triazole-4-carboxamide hydrochloride and acryloyl chloride, following a similar procedure to that described in Example 1, step 4: 1-(tert-butyl)-N-(2-methyl-4-(3-(3-(N-methylacrylamido)piperidin-1-yl)pyridin-4-yl)benzyl)-1H-1,2,3-triazole-4-carboxamide (54 mg, 37% yield). LCMS m/z=517.3 (M+H)+. ¹H NMR (400 MHz, DMSO-d₆) δ: 8.99 (s, 1H), 8.84 (s, 1H), 8.74-8.69 (m, 1H), 8.60-8.40 (m, 1H), 7.96-7.86 (m, 2H), 7.34 (d, 1H), 6.79-6.33 (m, 1H), 6.13-5.89 (m, 1H), 5.70-5.56 (m, 1H), 4.50 (d, 2H), 4.19-3.86 (m, 1H), 3.33-2.49 (m, 7H), 2.40 (s, 3H), 1.64 (m, 13H).

### Example 9: 1-(tert-butyl)-N-(2-methyl-4-(5-(3-(N-methylacrylamido)pyrrolidin-1-yl)pyrimidin-4-yl)benzyl)-1H-1,2,3-triazole-4-carboxamide

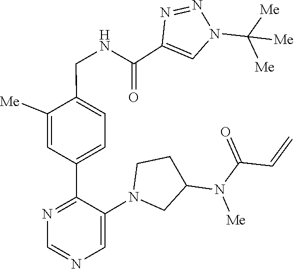 [SMILES: C=CC(=O)N(C)C1CCN(c2cncnc2-c2ccc(CNC(=O)c3cn(C(C)(C)C)nn3)c(C)c2)C1]

1-(tert-Butyl)-N-(2-methyl-4-(5-(3-(N-methylacrylamido)pyrrolidin-1-yl)pyrimidin-4-yl)benzyl)-1H-1,2,3-triazole-4-carboxamide was obtained from 5-iodo-4-methoxypyrimidine and tert-butyl cyclopentyl(methyl)carbamate, following the steps described for the synthesis of Example 8. The crude product was purified by prep HPLC (Method A2, organic gradient 23-53%) to give a white solid. LCMS m/z=503.2 (M+H)+. ¹H NMR (400 MHz, DMSO-d₆) δ: 8.95 (s, 1H), 8.65 (d, 2H), 8.35 (s, 1H), 7.48-7.42 (m, 2H), 7.27 (d, 1H), 6.76-6.66 (m, 1H), 6.03-5.80 (m, 1H), 5.07-4.68 (m, 1H), 4.46 (d, 2H), 3.12-2.63 (m, 8H), 2.34 (s, 3H), 2.00-1.80 (m, 2H), 1.61 (s, 9H).

### Example 10: 1-(tert-butyl)-N-(2-methyl-4-(3-(3-(N-methylacrylamido)-2-oxopiperidin-1-yl)pyridin-4-yl)benzyl)-1H-1,2,3-triazole-4-carboxamide

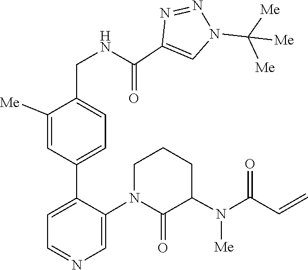 [SMILES: C=CC(=O)N(C)C1CCCN(c2cnccc2-c2ccc(CNC(=O)c3cn(C(C)(C)C)nn3)c(C)c2)C1=O]

### 1. Synthesis of tert-butyl (1-(4-chloropyridin-3-yl)-2-oxopiperidin-3-yl)carbamate

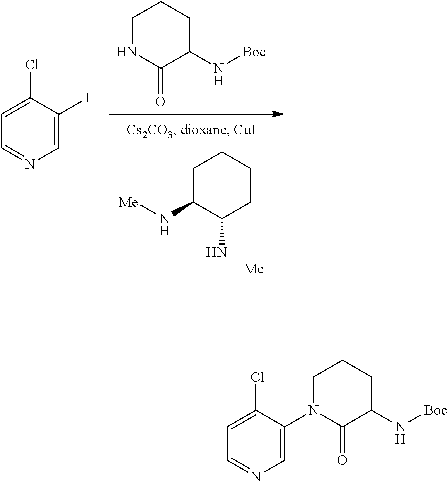 [SMILES: Clc1ccncc1I ; CC(C)(C)OC(=O)NC1CCCNC1=O ; CN[C@H]1CCCC[C@@H]1N ; "Me" ; CC(C)(C)OC(=O)NC1CCCN(c2cnccc2Cl)C1=O]

To a solution of 4-chloro-3-iodopyridine (500 mg, 2.09 mmol) in dioxane (6 mL) was added tert-butyl (2-oxopiperidin-3-yl)carbamate (447 mg, 2.09 mmol), CuI (159 mg, 836 μmol), Cs₂CO₃ (1.36 g, 4.18 mmol), and trans-N,N-dimethylcyclohexane-1,2-diamine (119 mg, 836 μmol) and the reaction was stirred at 90° C. under N₂ for 10 h. The cooled mixture was concentrated in vacuo and the residue was purified by silica gel column chromatography eluting with PE to EtOAc to give tert-butyl (1-(4-chloropyridin-3-yl)-2-oxopiperidin-3-yl)carbamate (200 mg, 29% yield) as yellow oil. LCMS m/z=326.2 (M+H)+.

### 2. Synthesis of tert-butyl (1-(4-chloropyridin-3-yl)-2-oxopiperidin-3-yl)(methyl)carbamate

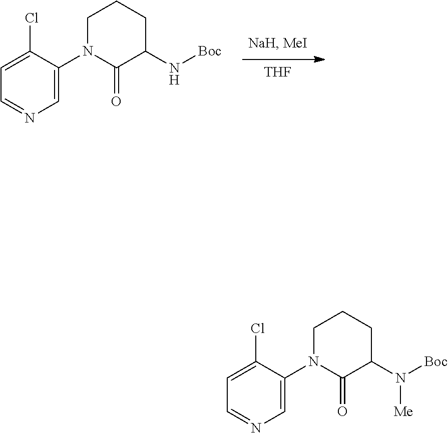 [SMILES: CC(C)(C)OC(=O)NC1CCCN(c2cnccc2Cl)C1=O ; "THF" ; CN(C(=O)OC(C)(C)C)C1CCCN(c2cnccc2Cl)C1=O]

To a solution of tert-butyl (1-(4-chloropyridin-3-yl)-2-oxopiperidin-3-yl)carbamate (200 mg, 614 μmol) in THF (20 mL) was added NaH (49 mg, 1.23 mmol, 60% purity) at 0° C. and the mixture was stirred for 10 mins at 0° C. MeI (174 mg, 1.23 mmol) was added, the ice bath was removed, and the reaction mixture was stirred at 25° C. for 6 h. The reaction was quenched with MeOH (2 mL) and concentrated in vacuo. The crude product was purified by silica gel column chromatography eluting with PE to EtOAc to give tert-butyl (1-(4-chloropyridin-3-yl)-2-oxopiperidin-3-yl)(methyl)carbamate (160 mg, 77% yield) as yellow oil. LCMS m/z=340.1 (M+H)+.

### 3. Synthesis of tert-butyl (1-(4-(4-((1-(tert-butyl)-1H-1,2,3-triazole-4-carboxamido)methyl)-3-methylphenyl)pyridin-3-yl)-2-oxopiperidin-3-yl)(methyl)carbamate

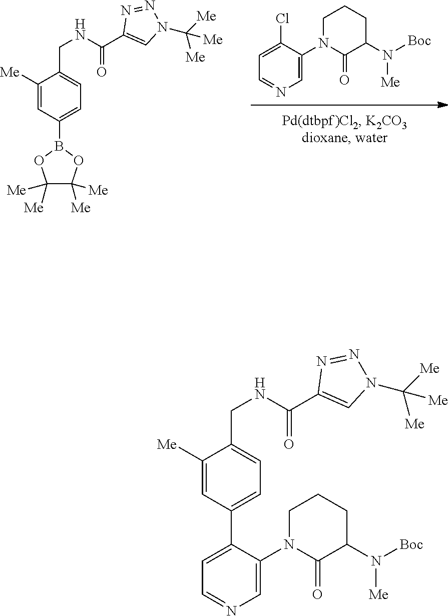 [SMILES: Cc1cc(B2OC(C)(C)C(C)(C)O2)ccc1CNC(=O)c1cn(C(C)(C)C)nn1 ; CN(C(=O)OC(C)(C)C)C1CCCN(c2cnccc2Cl)C1=O ; Cc1cc(-c2ccncc2N2CCCC(N(C)C(=O)OC(C)(C)C)C2=O)ccc1CNC(=O)c1cn(C(C)(C)C)nn1]

The compound was obtained from tert-butyl (1-(4-chloropyridin-3-yl)-2-oxopiperidin-3-yl)(methyl)carbamate and Intermediate 1: 1-(tert-butyl)-N-(2-methyl-4-(4,4,5,5-tetramethyl-1,3,2-dioxaborolan-2-yl)benzyl)-1H-1,2,3-triazole-4-carboxamide following the procedure described in Example 4, step 2: tert-butyl 7-(4-(4-((1-(tert-butyl)-1H-1,2,3-triazole-4-carboxamido)methyl)-3-methylphenyl)pyridin-3-yl)-2,7-diazaspiro[4.4]nonane-2-carboxylate. The crude product was purified by prep-TLC (EtOAc) to give tert-butyl (1-(4-(4-((1-(tert-butyl)-1H-1,2,3-triazole-4-carboxamido)methyl)-3-methylphenyl)pyridin-3-yl)-2-oxopiperidin-3-yl)(methyl)carbamate (130 mg, 69% yield) as yellow oil. LCMS m/z=576.4 (M+H)+.

### 4. Synthesis of 1-(tert-butyl)-N-(2-methyl-4-(3-(3-(methylamino)-2-oxopiperidin-1-yl)pyridin-4-yl)benzyl)-1H-1,2,3-triazole-4-carboxamide hydrochloride

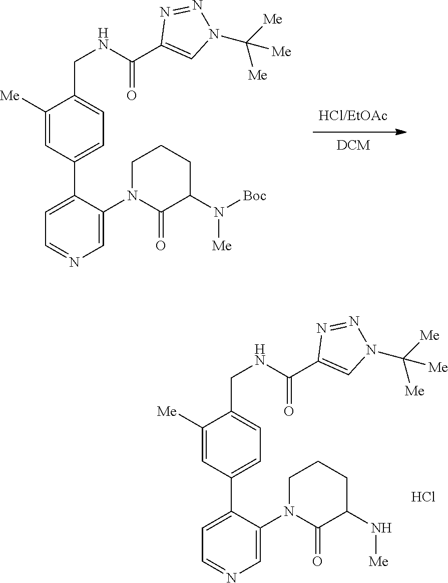

1-(tert-Butyl)-N-(2-methyl-4-(3-(3-(methylamino)-2-oxopiperidin-1-yl)pyridin-4-yl)benzyl)-1H-1,2,3-triazole-4-carboxamide hydrochloride was obtained as a yellow solid from tert-butyl (1-(4-(4-((1-(tert-butyl)-1H-1,2,3-triazole-4-carboxamido)methyl)-3-methylphenyl)pyridin-3-yl)-2-oxopiperidin-3-yl)(methyl)carbamate following the procedure described in Example 1, step 3: 1-(tert-butyl)-N-(2-methyl-4-(3-(3-(methylamino)piperidin-1-yl)pyridin-4-yl)benzyl)-1H-1,2,3-triazole-4-carboxamide hydrochloride. LCMS m/z=476.3 (M+H)+.

### 5. Synthesis of 1-(tert-butyl)-N-(2-methyl-4-(3-(3-(N-methylacrylamido)-2-oxopiperidin-1-yl)pyridin-4-yl)benzyl)-1H-1, 2,3-triazole-4-carboxamide

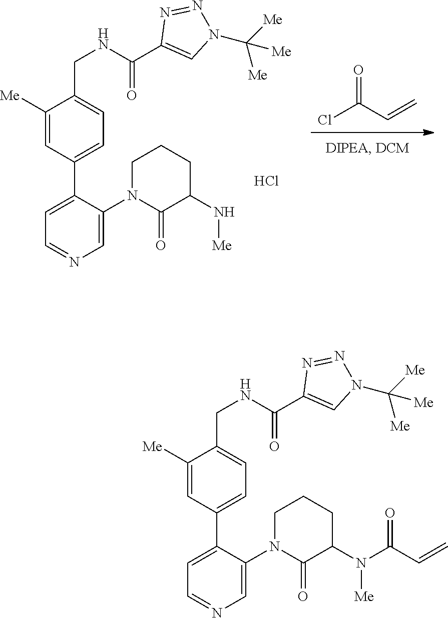

The compound was obtained from acryloyl chloride and 1-(tert-butyl)-N-(2-methyl-4-(3-(3-(methylamino)-2-oxopiperidin-1-yl)pyridin-4-yl)benzyl)-1H-1,2,3-triazole-4-carboxamide hydrochloride following the procedure described in Example 1, step 4: 1-(tert-butyl)-N-(2-methyl-4-(3-(3-(N-methylacrylamido)piperidin-1-yl)pyridin-4-yl)benzyl)-1H-1,2,3-triazole-4-carboxamide. The crude was purified by prep HPLC (Method A1, organic gradient 23-53%) to give 1-(tert-butyl)-N-(2-methyl-4-(3-(3-(N-methylacrylamido)-2-oxopiperidin-1-yl)pyridin-4-yl)benzyl)-1H-1,2,3-triazole-4-carboxamide as a white solid, (35 mg, 32% yield). LCMS m/z=530.2 (M+H)+. ¹H NMR (400 MHz, DMSO-d₆) δ: 8.98 (s, 1H), 8.71 (s, 1H), 8.58-8.48 (m, 2H), 7.47-7.32 (m, 2H), 7.24-7.13 (m, 2H), 6.81-6.52 (m, 1H), 6.20-5.94 (m, 1H), 5.75-5.56 (m, 1H), 4.55-4.45 (m, 2H), 3.64-3.48 (m, 1H), 3.32-2.72 (m, 5H), 2.39 (s, 3H), 2.26-1.72 (m, 4H), 1.64 (s, 9H).

### Example 11: 1-(tert-butyl)-N-(2-methyl-4-(3-(3-(N-methylacrylamido)-2-oxopyrrolidin-1-yl)pyridin-4-yl)benzyl)-1H-1,2,3-triazole-4-carboxamide

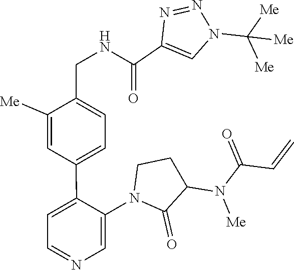 [SMILES: C=CC(=O)N(C)C1CCN(c2cnccc2-c2ccc(CNC(=O)c3cn(C(C)(C)C)nn3)c(C)c2)C1=O]

### 1. Synthesis of tert-butyl (1-(4-(4-((1-(tert-butyl)-1H-1,2,3-triazole-4-carboxamido)methyl)-3-methylphenyl)pyridin-3-yl)-2-oxopyrrolidin-3-yl)(methyl)carbamate

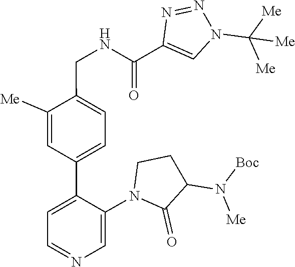 [SMILES: Cc1cc(-c2ccncc2N2CCC(N(C)C(=O)OC(C)(C)C)C2=O)ccc1CNC(=O)c1cn(C(C)(C)C)nn1]

The compound was obtained from 4-chloro-3-iodopyridine and tert-butyl (2-oxopyrrolidin-3-yl)carbamate, following the steps described in Example 10, steps 1 to 3. The crude product was purified by silica gel chromatography eluting with PE to EtOAc to provide tert-butyl (1-(4-(4-((1-(tert-butyl)-1H-1,2,3-triazole-4-carboxamido)methyl)-3-methylphenyl)pyridin-3-yl)-2-oxopyrrolidin-3-yl)(methyl)carbamate as a yellow oil (170 mg, 40% yield). LCMS m/z=562.3 (M+H)+.

### 2. Synthesis of 1-(tert-butyl)-N-(2-methyl-4-(3-(3-(N-methylacrylamido)-2-oxopyrrolidin-1-yl)pyridin-4-yl)benzyl)-1H-1,2,3-triazole-4-carboxamide

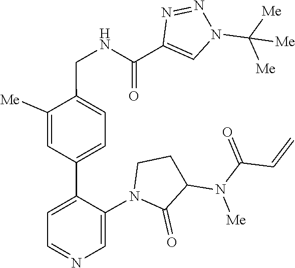 [SMILES: C=CC(=O)N(C)C1CCN(c2cnccc2-c2ccc(CNC(=O)c3cn(C(C)(C)C)nn3)c(C)c2)C1=O]

The compound was obtained from tert-butyl (1-(4-(4-((1-(tert-butyl)-1H-1,2,3-triazole-4-carboxamido)methyl)-3-methylphenyl)pyridin-3-yl)-2-oxopyrrolidin-3-yl)(methyl)carbamate following the steps described in Example 1, step 3 to 4. The crude was purified by prep HPLC (Method A1 organic gradient 22-52%) to give 1-(tert-butyl)-N-(2-methyl-4-(3-(3-(N-methylacrylamido)-2-oxopyrrolidin-1-yl)pyridin-4-yl)benzyl)-1H-1,2,3-triazole-4-carboxamide as a white solid (58 mg, 37% yield). LCMS m/z=516.2 (M+H)+. ¹H NMR (500 MHz, DMSO-d₆) δ: 9.02-8.96 (m, 1H), 8.74-8.68 (m, 1H), 8.61-8.54 (m, 2H), 7.44 (d, 1H), 7.34 (d, 1H), 7.27-7.17 (m, 2H), 6.77-6.66 (m, 1H), 6.19-6.04 (m, 1H), 5.75-5.64 (m, 1H), 5.25-4.95 (m, 1H), 4.47 (d, 2H), 3.65-3.37 (m, 2H), 3.31-2.71 (m, 3H), 2.39-2.36 (m, 3H), 2.30-2.10 (m, 1H), 2.03-1.89 (m, 1H), 1.64 (s, 9H).

### Example 12: 5-(tert-butyl)-N-(2-methyl-4-(3-(3-(N-methylacrylamido)piperidin-1-yl)pyridin-4-yl)benzyl)-1,2,4-oxadiazole-3-carboxamide

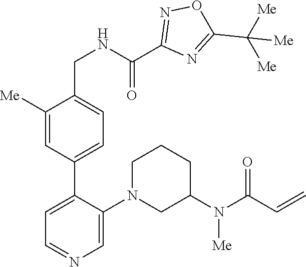 [SMILES: C=CC(=O)N(C)C1CCCN(c2cnccc2-c2ccc(CNC(=O)c3noc(C(C)(C)C)n3)c(C)c2)C1]

### 1. Synthesis of tert-butyl (1-(4-(4-((5-(tert-butyl)-1,2,4-oxadiazole-3-carboxamido)methyl)-3-methylphenyl)pyridin-3-yl)piperidin-3-yl)(methyl)carbamate

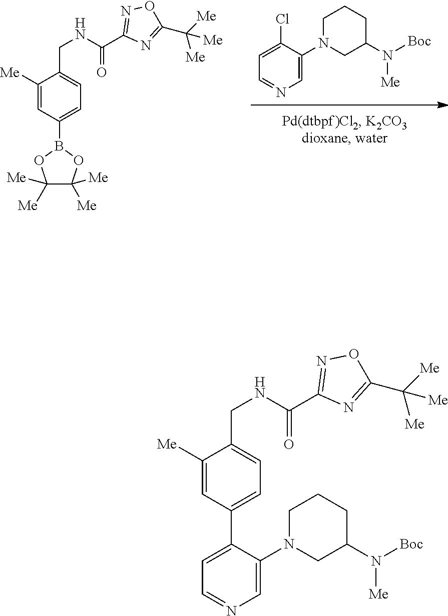 [SMILES: Cc1cc(B2OC(C)(C)C(C)(C)O2)ccc1CNC(=O)c1noc(C(C)(C)C)n1 ; CN(C(=O)OC(C)(C)C)C1CCCN(c2cnccc2Cl)C1 ; Cc1cc(-c2ccncc2N2CCCC(N(C)C(=O)OC(C)(C)C)C2)ccc1CNC(=O)c1noc(C(C)(C)C)n1]

The compound was obtained from Example 1, step 1: tert-butyl (1-(4-chloropyridin-3-yl)piperidin-3-yl)(methyl)carbamate and Intermediate 5: 5-(tert-butyl)-N-(2-methyl-4-(4,4,5,5-tetramethyl-1,3,2-dioxaborolan-2-yl)benzyl)-1,2,4-oxadiazole-3-carboxamide following the procedure described in Example 4, step 2: tert-butyl 7-(4-(4-((1-(tert-butyl)-1H-1,2,3-triazole-4-carboxamido)methyl)-3-methylphenyl)pyridin-3-yl)-2,7-diazaspiro[4.4]nonane-2-carboxylate. The crude was purified by silica gel chromatography (Combiflash®) eluting with PE to EtOAc to give tert-butyl (1-(4-(4-((5-(tert-butyl)-1,2,4-oxadiazole-3-carboxamido)methyl)-3-methylphenyl)pyridin-3-yl)piperidin-3-yl)(methyl)carbamate (170 mg, 25% yield) as a yellow oil. LCMS m/z=563.3 (M+H)+.

### 2. Synthesis of 5-(tert-butyl)-N-(2-methyl-4-(3-(3-(methylamino)piperidin-1-yl)pyridin-4-yl)benzyl)-1,2,4-oxadiazole-3-carboxamide hydrochloride

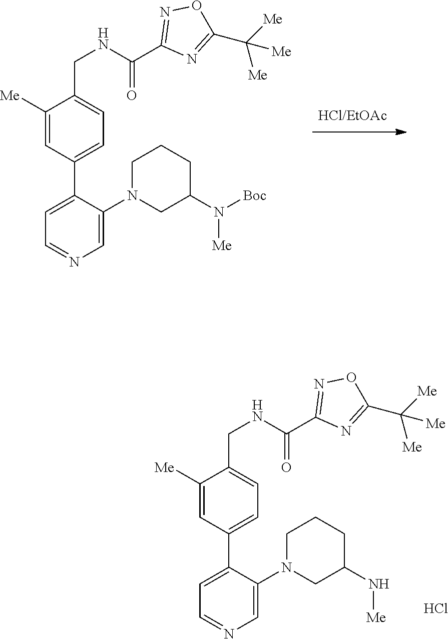 [SMILES: Cc1cc(-c2ccncc2N2CCCC(N(C)C(=O)OC(C)(C)C)C2)ccc1CNC(=O)c1noc(C(C)(C)C)n1 ; CNC1CCCN(c2cnccc2-c2ccc(CNC(=O)c3noc(C(C)(C)C)n3)c(C)c2)C1 ; Cl]

5-(tert-Butyl)-N-(2-methyl-4-(3-(3-(methylamino)piperidin-1-yl)pyridin-4-yl)benzyl)-1,2,4-oxadiazole-3-carboxamide hydrochloride was obtained as a yellow oil from tert-butyl (1-(4-(4-((5-(tert-butyl)-1,2,4-oxadiazole-3-carboxamido)methyl)-3-methylphenyl)pyridin-3-yl)piperidin-3-yl)(methyl)carbamate, following the procedure described in Example 1, step 3: 1-(tert-butyl)-N-(2-methyl-4-(3-(3-(methylamino)piperidin-1-yl)pyridin-4-yl)benzyl)-1H-1,2,3-triazole-4-carboxamide hydrochloride (190 mg, crude). LCMS m/z=463.3 (M+H)+.

### 3. Synthesis of 5-(tert-butyl)-N-(2-methyl-4-(3-(3-(N-methylacrylamido)piperidin-1-yl)pyridin-4-yl)benzyl)-1,2,4-oxadiazole-3-carboxamide

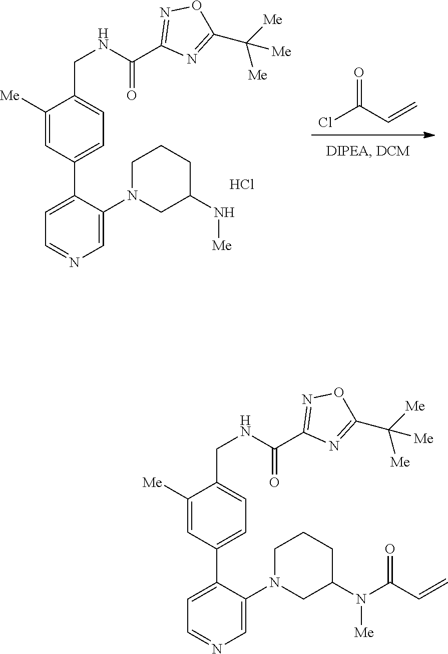

The compound was obtained from 5-(tert-butyl)-N-(2-methyl-4-(3-(3-(methylamino)piperidin-1-yl)pyridin-4-yl)benzyl)-1,2,4-oxadiazole-3-carboxamide hydrochloride and acryloyl chloride following the procedure described in Example 3, step 4. The crude was purified by prep-HPLC (Method B, organic gradient 32-62%) to give 5-(tert-butyl)-N-(2-methyl-4-(3-(3-(N-methylacrylamido)piperidin-1-yl)pyridin-4-yl)benzyl)-1,2,4-oxadiazole-3-carboxamide as a pale yellow solid (50 mg, 24% yield). LCMS m/z=539.4 (M+Na)+. ¹H NMR (500 MHz, MeOH-d₄) δ: 8.32-8.21 (m, 2H), 7.57-7.42 (m, 3H), 7.27-7.25 (m, 1H), 6.67-6.08 (m, 2H), 5.72-5.67 (m, 1H), 4.90-4.71 (m, 2H), 4.69-3.77 (m, 1H), 3.34-2.97 (m, 2H), 2.83-2.72 (m, 5H), 2.47 (s, 3H), 2.06-1.67 (m, 4H), 1.50 (s, 9H).

### Example 13: 3-(tert-butyl)-N-(2-methyl-4-(3-(3-(N-methylacrylamido)piperidin-1-yl)pyridin-4-yl)benzyl)-1,2,4-oxadiazole-5-carboxamide

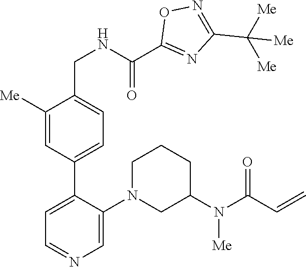 [SMILES: C=CC(=O)N(C)C1CCCN(c2cnccc2-c2ccc(CNC(=O)c3nc(C(C)(C)C)no3)c(C)c2)C1]

### 1. Synthesis of tert-butyl (1-(4-(4-((3-(tert-butyl)-1,2,4-oxadiazole-5-carboxamido)methyl)-3-methylphenyl)pyridin-3-yl)piperidin-3-yl)(methyl)carbamate

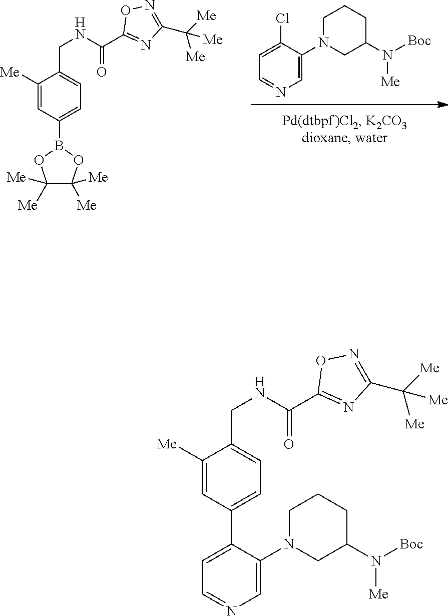 [SMILES: Cc1cc(B2OC(C)(C)C(C)(C)O2)ccc1CNC(=O)c1nc(C(C)(C)C)no1 ; CN(C(=O)OC(C)(C)C)C1CCCN(c2cnccc2Cl)C1 ; Cc1cc(-c2ccncc2N2CCCC(N(C)C(=O)OC(C)(C)C)C2)ccc1CNC(=O)c1nc(C(C)(C)C)no1]

tert-Butyl (1-(4-(4-((3-(tert-butyl)-1,2,4-oxadiazole-5-carboxamido)methyl)-3-methylphenyl)pyridin-3-yl)piperidin-3-yl)(methyl)carbamate was obtained as a yellow solid from Intermediate 2: 3-(tert-butyl)-N-(2-methyl-4-(4,4,5,5-tetramethyl-1,3,2-dioxaborolan-2-yl)benzyl)-1,2,4-oxadiazole-5-carboxamide and Example 1, step 1: tert-butyl (1-(4-chloropyridin-3-yl)piperidin-3-yl)(methyl)carbamate following the procedure described in Example 12, step 1: tert-butyl (1-(4-(4-((5-(tert-butyl)-1,2,4-oxadiazole-3-carboxamido)methyl)-3-methylphenyl)pyridin-3-yl)piperidin-3-yl)(methyl)carbamate, (345 mg, 68% yield). LCMS m/z=563.3 (M+H)+.

### 2. Synthesis of 3-(tert-butyl)-N-(2-methyl-4-(3-(3-(methylamino)piperidin-1-yl)pyridin-4-yl)benzyl)-1,2,4-oxadiazole-5-carboxamide hydrochloride

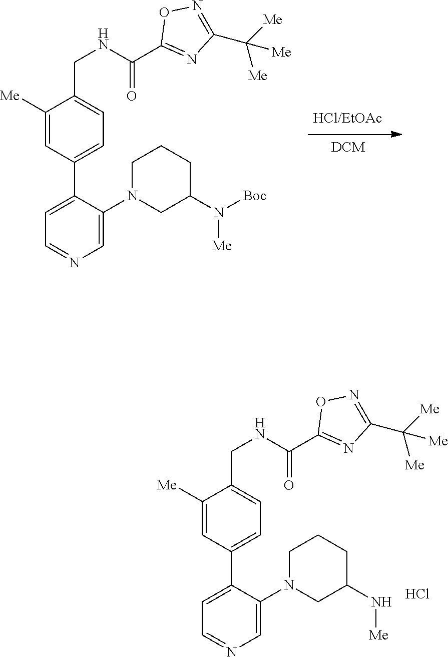

3-(tert-Butyl)-N-(2-methyl-4-(3-(3-(methylamino)piperidin-1-yl)pyridin-4-yl)benzyl)-1,2,4-oxadiazole-5-carboxamide hydrochloride was obtained from tert-butyl (1-(4-(4-((3-(tert-butyl)-1,2,4-oxadiazole-5-carboxamido)methyl)-3-methylphenyl)pyridin-3-yl)piperidin-3-yl)(methyl)carbamate following the procedure described in Example 1, step 3: 1-(tert-butyl)-N-(2-methyl-4-(3-(3-(methylamino)piperidin-1-yl)pyridin-4-yl)benzyl)-1H-1,2,3-triazole-4-carboxamide hydrochloride. LCMS m/z=463.3 (M+H)+.

### 3. Synthesis of 3-(tert-butyl)-N-(2-methyl-4-(3-(3-(N-methylacrylamido)piperidin-1-yl)pyridin-4-yl)benzyl)-1,2,4-oxadiazole-5-carboxamide

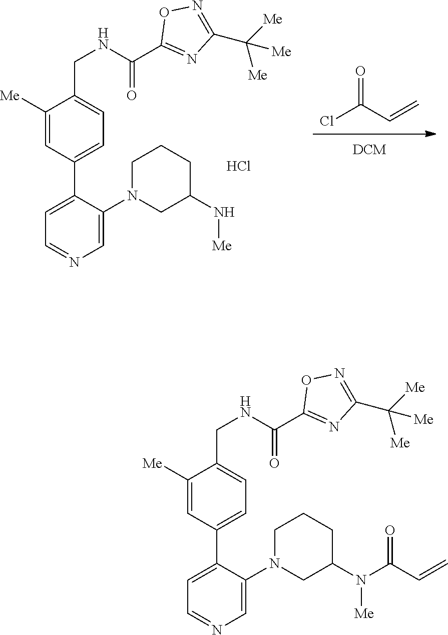

The compound was obtained from acryloyl chloride and 3-(tert-butyl)-N-(2-methyl-4-(3-(3-(methylamino)piperidin-1-yl)pyridin-4-yl)benzyl)-1,2,4-oxadiazole-5-carboxamide hydrochloride following the procedure described in Example 2, step 4: 1-(tert-butyl)-N-(2-methyl-4-(3-(3-(N-methylacrylamido)pyrrolidin-1-yl)pyridin-4-yl)benzyl)-1H-1,2,3-triazole-4-carboxamide. The crude product was purified by prep-HPLC (Method B, organic gradient 39-69%) to give 3-(tert-butyl)-N-(2-methyl-4-(3-(3-(N-methylacrylamido)piperidin-1-yl)pyridin-4-yl)benzyl)-1,2,4-oxadiazole-5-carboxamide (75 mg, 28% yield) as a yellow solid. LCMS m/z=517.3 (M+H)+. ¹H NMR (400 MHz, DMSO-d₆) δ: 9.83 (s, 1H), 8.35 (s, 1H), 8.26 (s, 1H), 7.59-7.44 (m, 2H), 7.40-7.35 (m, 1H), 7.18 (s, 1H), 6.90-5.88 (m, 2H), 5.83-5.27 (m, 1H), 4.50 (s, 2H), 4.42-3.66 (m, 1H), 3.13-2.62 (m, 7H), 2.39 (s, 3H), 1.70-1.47 (m, 4H), 1.36 (s, 9H).

### Example 14: 5-(tert-butyl)-N-(2-methyl-4-(3-(3-(N-methylacrylamido)piperidin-1-yl)pyridin-4-yl)benzyl)isoxazole-3-carboxamide

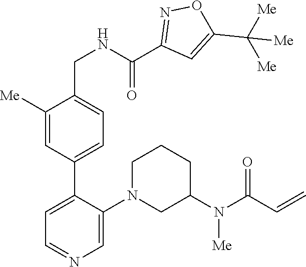 [SMILES: C=CC(=O)N(C)C1CCCN(c2cnccc2-c2ccc(CNC(=O)c3cc(C(C)(C)C)on3)c(C)c2)C1]

### 1. Synthesis of tert-butyl (1-(4-(4-((5-(tert-butyl)isoxazole-3-carboxamido)methyl)-3-methylphenyl)pyridin-3-yl)piperidin-3-yl)(methyl)carbamate

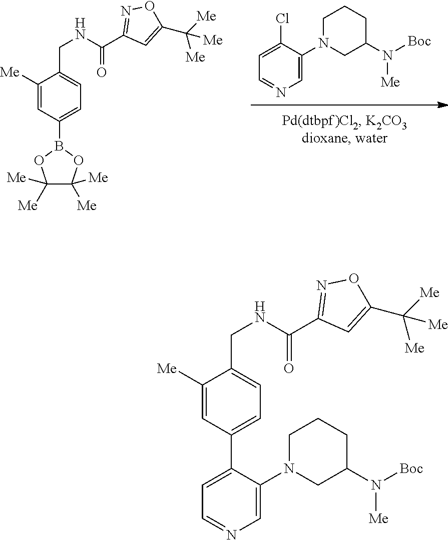 [SMILES: Cc1cc(B2OC(C)(C)C(C)(C)O2)ccc1CNC(=O)c1cc(C(C)(C)C)on1 ; CN(C(=O)OC(C)(C)C)C1CCCN(c2cnccc2Cl)C1 ; Cc1cc(-c2ccncc2N2CCCC(N(C)C(=O)OC(C)(C)C)C2)ccc1CNC(=O)c1cc(C(C)(C)C)on1]

tert-Butyl (1-(4-(4-((5-(tert-butyl)isoxazole-3-carboxamido)methyl)-3-methylphenyl)pyridin-3-yl)piperidin-3-yl)(methyl)carbamate was obtained as a yellow solid, from Intermediate 3:5-(tert-butyl)-N-(2-methyl-4-(4,4,5,5-tetramethyl-1,3,2-dioxaborolan-2-yl)benzyl)isoxazole-3-carboxamide and Example 1, step 1: tert-butyl (1-(4-chloropyridin-3-yl)piperidin-3-yl)(methyl)carbamate, following the procedure described in Example 12, step 1: tert-butyl (1-(4-(4-((5-(tert-butyl)-1,2,4-oxadiazole-3-carboxamido)methyl)-3-methylphenyl)pyridin-3-yl)piperidin-3-yl)(methyl)carbamate (300 mg, 55% yield). LCMS m/z=562.4 (M+H)+.

### 2. Synthesis of 5-(tert-butyl)-N-(2-methyl-4-(3-(3-(N-methylacrylamido)piperidin-1-yl)pyridin-4-yl)benzyl)isoxazole-3-carboxamide

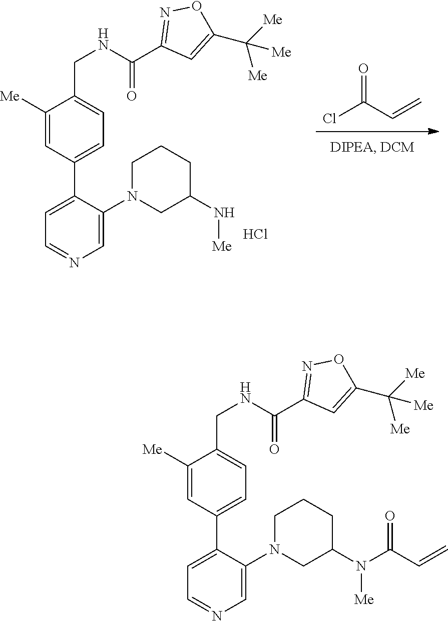

5-(tert-Butyl)-N-(2-methyl-4-(3-(3-(N-methylacrylamido)piperidin-1-yl)pyridin-4-yl)benzyl)isoxazole-3-carboxamide was obtained as a yellow solid, from tert-butyl (1-(4-(4-((5-(tert-butyl)isoxazole-3-carboxamido)methyl)-3-methylphenyl)pyridin-3-yl)piperidin-3-yl)(methyl)carbamate following the procedure described in Example 2, steps 3 and 4, except the prep HPLC gradient was (41 to 71%) (80 mg, 41% yield). LCMS m/z=516.3 (M+H)+. ¹H NMR (400 MHz, DMSO-d₆) δ: 9.19-9.13 (m, 1H), 8.31 (s, 1H), 8.22 (s, 1H), 7.51-7.42 (m, 2H), 7.29 (d, 1H), 7.14 (s, 1H), 6.70-6.05 (m, 2H), 6.01-5.52 (m, 2H), 4.49-4.35 (m, 2H), 3.65 (m, 1H), 3.07-2.55 (m, 7H), 2.34 (s, 3H), 1.66-1.42 (m, 4H), 1.30 (s, 9H).

### Example 15 (S)-1-(tert-butyl)-N-(2-methyl-4-(3-(3-(N-methylacrylamido)piperidin-1-yl)pyridin-4-yl)benzyl)-1H-1,2,3-triazole-4-carboxamide

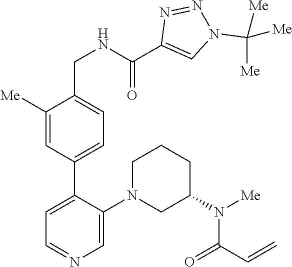 [SMILES: C=CC(=O)N(C)[C@H]1CCCN(c2cnccc2-c2ccc(CNC(=O)c3cn(C(C)(C)C)nn3)c(C)c2)C1]

### 1. Synthesis of tert-butyl (S)-(1-(4-chloropyridin-3-yl)piperidin-3-yl)(methyl)carbamate

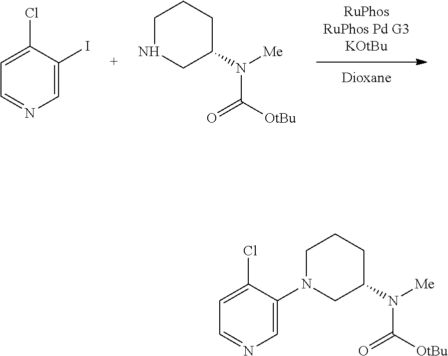 [SMILES: Clc1ccncc1I ; CN(C(=O)OC(C)(C)C)[C@H]1CCCNC1 ; CN(C(=O)OC(C)(C)C)[C@H]1CCCN(c2cnccc2Cl)C1]

A mixture of 4-chloro-3-iodopyridine (250 mg, 1.04 mmol), tert-butyl (S)-methyl(piperidin-3-yl)carbamate (200 mg, 933 μmol), RuPhos (40 mg, 85 μmol), RuPhos Pd G3 (78 mg, 85 μmol) and KOtBu (143 mg, 1.27 mmol) was purged with N₂, degassed dioxane was added, and the resulting mixture was heated under reflux for 2 h. The cooled reaction mixture was diluted with EtOAc and filtered through Celite®. The filtrate was washed with water and brine, dried over Na₂SO₄, filtered and evaporated under reduced pressure. The resulting dark oil was purified by silica gel column chromatography eluting with EtOAc/EtOH (3:1) in heptane (0/100 to 50/50) to give tert-butyl (S)-(1-(4-chloropyridin-3-yl)piperidin-3-yl)(methyl)carbamate as a pale yellow oil (204 mg, 74% yield). LCMS m/z=326.1 (M+H)+.

### 2. Synthesis of tert-butyl (1-(tert-butyl)-1H-1,2,3-triazole-4-carbonyl)(2-methyl-4-(4,4,5,5-tetramethyl-1,3,2-dioxaborolan-2-yl)benzyl)carbamate

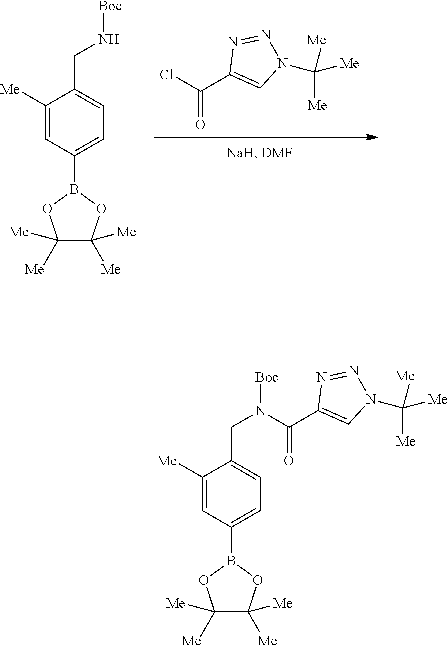 [SMILES: Cc1cc(B2OC(C)(C)C(C)(C)O2)ccc1CNC(=O)OC(C)(C)C ; CC(C)(C)n1cc(C(=O)Cl)nn1 ; Cc1cc(B2OC(C)(C)C(C)(C)O2)ccc1CN(C(=O)OC(C)(C)C)C(=O)c1cn(C(C)(C)C)nn1]

To a solution of tert-butyl (2-methyl-4-(4,4,5,5-tetramethyl-1,3,2-dioxaborolan-2-yl)benzyl)carbamate (406 mg, 1.17 mmol) in DMF (5 mL) was added NaH (94 mg, 2.34 mmol, 60% purity) at rt and the solution was stirred for 1 h under N₂. A solution of 1-tert-butyltriazole-4-carbonyl chloride (220 mg, 1.17 mmol) in DMF (1 mL) was added dropwise and the reaction stirred for a further hour. The reaction was quenched with water and diluted with EtOAc and the layers were separated. The organic phase was dried over Na₂SO₄, filtered and concentrated in vacuo. The crude was purified by silica gel column chromatography eluting with EtOAc/EtOH (3:1) in heptane (0/100 to 60/40) to give tert-butyl (1-(tert-butyl)-1H-1,2,3-triazole-4-carbonyl)(2-methyl-4-(4,4,5,5-tetramethyl-1,3,2-dioxaborolan-2-yl)benzyl)carbamate (433 mg, 74% yield) as a colorless oil. LCMS m/z=399.2 (M-Boc+H)+.

### 3. Synthesis of tert-butyl (S)-(1-(4-(4-((N-(tert-butoxycarbonyl)-1-(tert-butyl)-1H-1,2,3-triazole-4-carboxamido)methyl)-3-methylphenyl)pyridin-3-yl)piperidin-3-yl)(methyl)carbamate

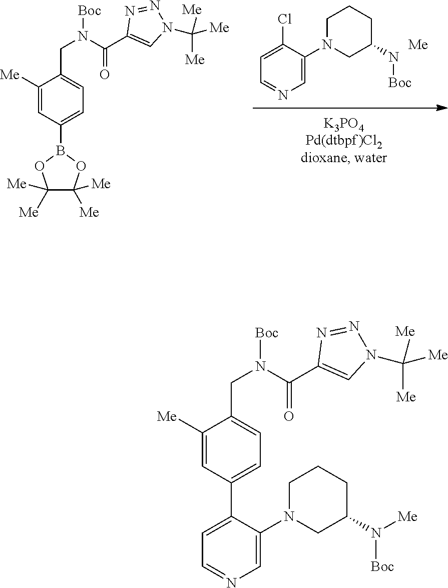 [SMILES: Cc1cc(B2OC(C)(C)C(C)(C)O2)ccc1CN(C(=O)OC(C)(C)C)C(=O)c1cn(C(C)(C)C)nn1 ; CC(C)(C)OC(=O)N[C@H]1CCCN(c2cnccc2Cl)C1 ; Cc1cc(-c2ccncc2N2CCC[C@H](NC(=O)OC(C)(C)C)C2)ccc1CN(C(=O)OC(C)(C)C)C(=O)c1cn(C(C)(C)C)nn1 ; "Me" ; "Me"]

A mixture of tert-butyl (S)-(1-(4-chloropyridin-3-yl)piperidin-3-yl)(methyl)carbamate (75 mg, 229 μmol), tert-butyl (1-(tert-butyl)-1H-1,2,3-triazole-4-carbonyl)(2-methyl-4-(4,4,5,5-tetramethyl-1,3,2-dioxaborolan-2-yl)benzyl)carbamate (120 mg, 241 μmol), K₃PO₄ (97 mg, 459 μmol) and Pd(dtbpf)Cl₂ (27 mg, 37 μmol) was purged with N₂. Dioxane (4.5 mL) and water (500 μL) were added via syringe and the reaction was sealed and heated under reflux for 2 h. The cooled reaction was diluted with EtOAc, filtered through Celite®, and the filtrate was concentrated in vacuo. The residue was purified by silica gel column chromatography EtOAc/EtOH (3:1) in heptane (0/100 to 40/60) to give tert-butyl (S)-(1-(4-(4-((N-(tert-butoxycarbonyl)-1-(tert-butyl)-1H-1,2,3-triazole-4-carboxamido)methyl)-3-methylphenyl)pyridin-3-yl)piperidin-3-yl)(methyl)carbamate as a pale yellow oil (58 mg, 38% yield). LCMS m/z=562.2 (M-Boc+H)+.

### 4. Synthesis of (S)-1-(tert-butyl)-N-(2-methyl-4-(3-(3-(methylamino)piperidin-1-yl)pyridin-4-yl)benzyl)-1H-1,2,3-triazole-4-carboxamide

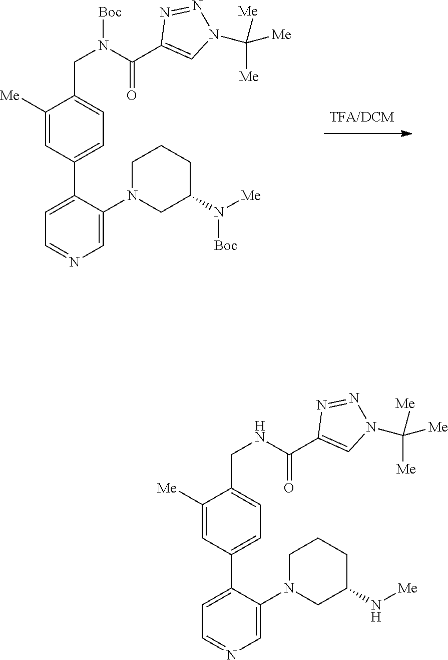

To a solution of tert-butyl (S)-(1-(4-(4-((N-(tert-butoxycarbonyl)-1-(tert-butyl)-1H-1,2,3-triazole-4-carboxamido)methyl)-3-methylphenyl)pyridin-3-yl)piperidin-3-yl)(methyl)carbamate (52 mg, 79 μmol) in DCM (3 mL) was added TFA (1.49 g, 13.1 mmol) and the reaction was stirred at rt for 18 h. The mixture was evaporated under reduced pressure and the residue was purified by silica gel column chromatography eluting with 5% NH₄OH/MeOH in DCM (0/100 to 10/90) to give (S)-1-(tert-butyl)-N-(2-methyl-4-(3-(3-(methylamino)piperidin-1-yl)pyridin-4-yl)benzyl)-1H-1,2,3-triazole-4-carboxamide (30 mg, 83% yield). LCMS m/z=462.2 (M+H)+. ¹H NMR (500 MHz, CDCl₃) δ: 8.31-8.21 (m, 2H), 8.15 (s, 1H), 7.48 (br t, 1H), 7.39-7.31 (m, 3H), 7.05 (d, 1H), 5.41 (br s, 1H), 4.68-4.53 (m, 2H), 3.14 (br d, 1H), 3.05 (br d, 1H), 2.71-2.62 (m, 1H), 2.55 (br t, 1H), 2.47-2.39 (m, 1H), 2.36 (s, 3H), 2.14 (s, 3H), 1.87 (br dd, 1H), 1.63 (s, 9H), 1.53-1.40 (m, 1H), 1.37-1.25 (m, 1H), 1.24-1.14 (m, 1H).

### 5. Synthesis of(S)-1-(tert-butyl)-N-(2-methyl-4-(3-(3-(N-methylacrylamido)piperidin-1-yl)pyridin-4-yl)benzyl)-1H-1, 2,3-triazole-4-carboxamide

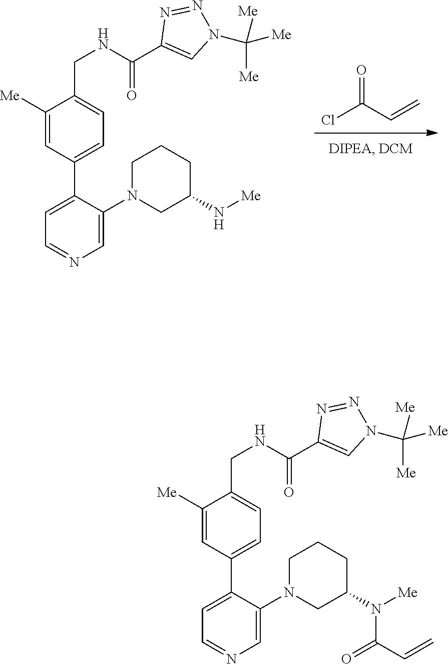

To a solution of (S)-1-(tert-butyl)-N-(2-methyl-4-(3-(3-(methylamino)piperidin-1-yl)pyridin-4-yl)benzyl)-1H-1,2,3-triazole-4-carboxamide (30 mg, 64.99 μmol) and DIPEA (25.20 mg, 194.97 μmol) in DCM (1 mL) at 0° C. was added acryloyl chloride (7.35 mg, 81.24 μmol) and the reaction stirred for 30 min. The reaction mixture was diluted with DCM (5 mL) and washed with sat. aq. NH₄Cl, water and brine. The organic phase was dried over Na₂SO₄, filtered and concentrated in vacuo. The crude product was purified by silica gel column chromatography eluting with MeOH/5% NH₄OH in DCM (0/100 to 15/85) to give (S)-1-(tert-butyl)-N-(2-methyl-4-(3-(3-(N-methylacrylamido)piperidin-1-yl)pyridin-4-yl)benzyl)-1H-1,2,3-triazole-4-carboxamide (21 mg, 62.67% yield) as an off white solid. LCMS m/z=516.2 (M+H)+. ¹H NMR (500 MHz, MeCN-d₃) S: 8.33 (br s, 1H), 8.24 (br s, 1H), 8.05-7.78 (m, 1H), 7.60-7.33 (m, 3H), 7.15 (br s, 1H), 6.65-6.10 (m, 1H), 6.10-5.95 (m, 1H), 5.63-5.51 (m, 1H), 4.76-4.45 (m, 2H), 4.43-3.65 (m, 1H), 3.30-2.95 (m, 2H), 2.89-2.72 (m, 3H), 2.68 (br d, 2H), 2.41 (s, 3H), 2.15 (s, 3H), 1.74 (br s, 1H), 1.67 (s, 9H), 1.63 (br s, 1H).

### Example 16: 1-(tert-butyl)-N-(4-(3-(3-(N-ethylacrylamido)piperidin-1-yl)pyridin-4-yl)-2-methylbenzyl)-1H-1,2,3-triazole-4-carboxamide

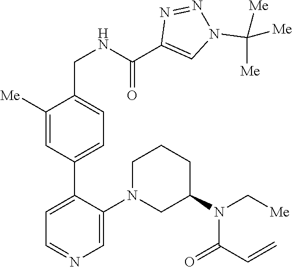 [SMILES: C=CC(=O)N(CC)[C@@H]1CCCN(c2cnccc2-c2ccc(CNC(=O)c3cn(C(C)(C)C)nn3)c(C)c2)C1]

### 1. Synthesis of tert-butyl (1-(4-chloropyridin-3-yl)piperidin-3-yl)(ethyl)carbamate

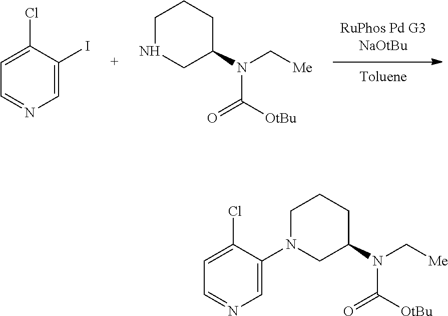 [SMILES: Clc1ccncc1I ; CCN(C(=O)OC(C)(C)C)[C@@H]1CCCNC1 ; CCN(C(=O)OC(C)(C)C)[C@@H]1CCCN(c2cnccc2Cl)C1]

A mixture of tert-butyl N-ethyl-N-(3-piperidyl)carbamate (200 mg, 876 μmol), 4-chloro-3-iodopyridine (250 mg, 1.04 mmol), RuPhos Pd G3 (88 mg, 105 μmol) and NaOtBu (168 mg, 1.75 mmol) in toluene (4 mL) was degassed with N₂, and the reaction vessel sealed and then stirred at 100° C. for 18 h. The cooled reaction mixture was concentrated in vacuo and the residue purified by silica gel column chromatography eluting with EtOAc in heptanes (0/100 to 50/50) to give tert-butyl (1-(4-chloropyridin-3-yl)piperidin-3-yl)(ethyl)carbamate (204.20 mg, 68.59% yield) as colorless oil. LCMS m/z=340.2 (M+H)+.

### 2. Synthesis of 1-(tert-butyl)-N-(4-(3-(3-(N-ethylacrylamido)piperidin-1-yl)pyridin-4-yl)-2-methylbenzyl)-1H-1,2,3-triazole-4-carboxamide

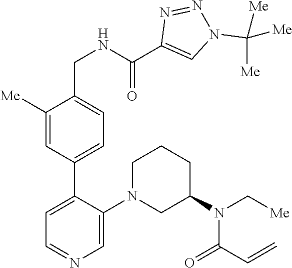 [SMILES: C=CC(=O)N(CC)[C@@H]1CCCN(c2cnccc2-c2ccc(CNC(=O)c3cn(C(C)(C)C)nn3)c(C)c2)C1]

The compound was obtained from tert-butyl (1-(4-chloropyridin-3-yl)piperidin-3-yl)(ethyl)carbamate and Intermediate 1: 1-(tert-butyl)-N-(2-methyl-4-(4,4,5,5-tetramethyl-1,3,2-dioxaborolan-2-yl)benzyl)-1H-1,2,3-triazole-4-carboxamide, following a similar procedure to that described in Example 4, steps 2 to 4. The crude was purified by silica gel column chromatography eluting with EtOAc/heptanes/MeOH (0/100/0 to 100/0/0 to 90/0/10) to give 1-(tert-butyl)-N-(4-(3-(3-(N-ethylacrylamido)piperidin-1-yl)pyridin-4-yl)-2-methylbenzyl)-1H-1,2,3-triazole-4-carboxamide (58 mg, 50% yield). LCMS m/z=530.4 (M+H)+. ¹H NMR (400 MHz, MeOH-d₄) S: 8.89-8.78 (m, 1H), 8.48 (s, 1H), 8.31-8.17 (m, 2H), 7.56-7.53 (m, 1H), 7.53-7.40 (m, 2H), 7.28-7.22 (m, 1H), 6.67-5.97 (m, 2H), 5.72-5.59 (m, 1H), 4.73-4.58 (m, 2H), 4.19-3.70 (m, 1H), 3.29-2.98 (m, 3H), 2.93-2.73 (m, 1H), 2.70-2.54 (m, 1H), 2.45 (s, 3H), 1.88-1.73 (m, 2H), 1.72-1.70 (m, 9H), 1.70-1.65 (m, 1H), 1.63-1.51 (m, 1H), 1.06 (td, 3H).

### Example 17: 1-(tert-butyl)-N-(4-(3-(3-(N-cyclopropylacrylamido)piperidin-1-yl)pyridin-4-yl)-2-methylbenzyl)-1H-1,2,3-triazole-4-carboxamide

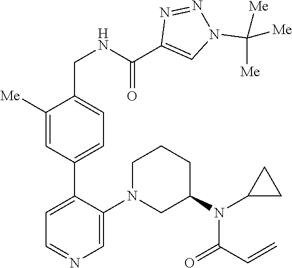 [SMILES: C=CC(=O)N(C1CC1)[C@@H]1CCCN(c2cnccc2-c2ccc(CNC(=O)c3cn(C(C)(C)C)nn3)c(C)c2)C1]

1-(tert-Butyl)-N-(4-(3-(3-(N-cyclopropylacrylamido)piperidin-1-yl)pyridin-4-yl)-2-methylbenzyl)-1H-1,2,3-triazole-4-carboxamide was obtained as a colorless oil, from tert-butyl N-cyclopropyl-N-(3-piperidyl)carbamate and 4-chloro-3-iodopyridine following the steps described in Example 16. LCMS m/z=542.4 (M+H)+. ¹H NMR (400 MHz, MeOH-d₄) δ: 8.47 (s, 1H), 8.25 (s, 1H), 8.19 (d, 1H), 7.59-7.54 (m, 1H), 7.54-7.48 (m, 1H), 7.46-7.40 (m, 1H), 7.26-7.20 (m, 1H), 6.94-6.75 (m, 1H), 6.11 (dd, 1H), 5.66-5.59 (m, 1H), 4.70-4.59 (m, 2H), 3.85-3.70 (m, 1H), 3.18-3.10 (m, 1H), 3.08 (d, 2H), 2.68-2.59 (m, 1H), 2.59-2.52 (m, 1H), 2.46 (s, 3H), 2.16-2.04 (m, 1H), 1.84-1.76 (m, 1H), 1.75-1.66 (m, 10H), 1.65-1.50 (m, 1H), 0.89-0.77 (m, 2H), 0.72-0.61 (m, 1H), 0.51-0.41 (m, 1H).

### Example 18: 3-(tert-butyl)-N-(3-fluoro-2-methyl-4-(3-(3-(N-methylacrylamido)piperidin-1-yl)pyridin-4-yl)benzyl)-1,2,4-oxadiazole-5-carboxamide

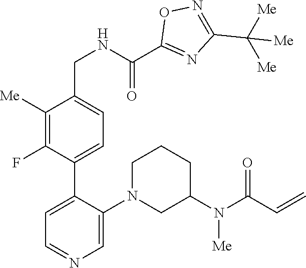 [SMILES: C=CC(=O)N(C)C1CCCN(c2cnccc2-c2ccc(CNC(=O)c3nc(C(C)(C)C)no3)c(C)c2F)C1]

### 1. Synthesis of tert-butyl (1-(4-(4-((3-(tert-butyl)-1,2,4-oxadiazole-5-carboxamido)methyl)-2-fluoro-3-methylphenyl)pyridin-3-yl)piperidin-3-yl)(methyl)carbamate

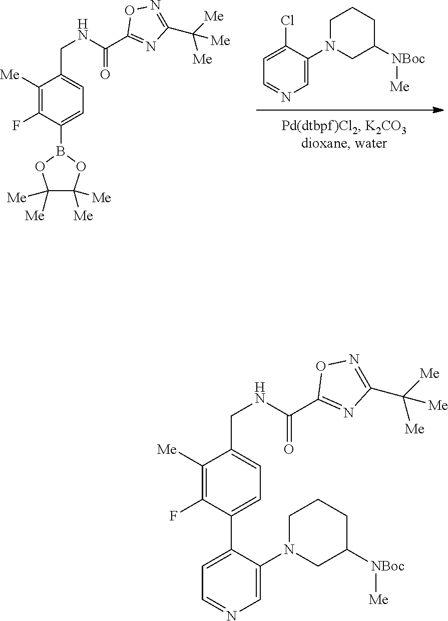 [SMILES: Cc1c(CNC(=O)c2nc(C(C)(C)C)no2)ccc(B2OC(C)(C)C(C)(C)O2)c1F ; CN(C(=O)OC(C)(C)C)C1CCCN(c2cnccc2Cl)C1 ; Cc1c(CNC(=O)c2nc(C(C)(C)C)no2)ccc(-c2ccncc2N2CCCC(N(C)C(=O)OC(C)(C)C)C2)c1F]

tert-Butyl (1-(4-(4-((3-(tert-butyl)-1,2,4-oxadiazole-5-carboxamido)methyl)-2-fluoro-3-methylphenyl)pyridin-3-yl)piperidin-3-yl)(methyl)carbamate was obtained from 3-(tert-butyl)-N-(3-fluoro-2-methyl-4-(4,4,5,5-tetramethyl-1,3,2-dioxaborolan-2-yl)benzyl)-1,2,4-oxadiazole-5-carboxamide and Example 1, step 1: tert-butyl (1-(4-chloropyridin-3-yl)piperidin-3-yl)(methyl)carbamate as a yellow oil, following a similar procedure to that described in Example 12, step 1: tert-butyl (1-(4-(4-((5-(tert-butyl)-1,2,4-oxadiazole-3-carboxamido)methyl)-3-methylphenyl)pyridin-3-yl)piperidin-3-yl)(methyl)carbamate. LCMS m/z=581.3 (M+H)+.

### 2. Synthesis of 3-(tert-butyl)-N-(3-fluoro-2-methyl-4-(3-(3-(methylamino)piperidin-1-yl)pyridin-4-yl)benzyl)-1,2,4-oxadiazole-5-carboxamide hydrochloride

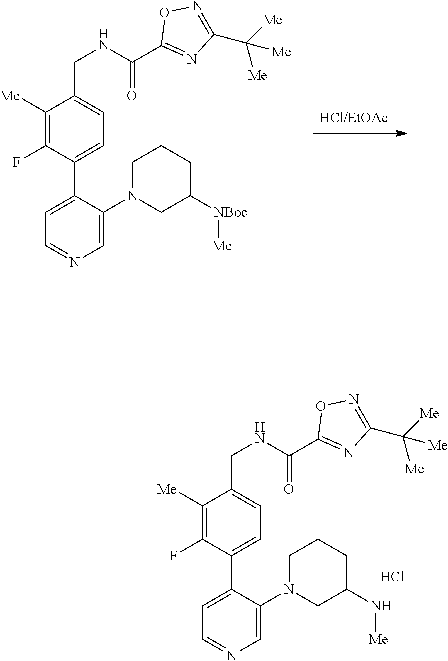 [SMILES: Cc1c(CNC(=O)c2nc(C(C)(C)C)no2)ccc(-c2ccncc2N2CCCC(N(C)C(=O)OC(C)(C)C)C2)c1F ; CNC1CCCN(c2cnccc2-c2ccc(CNC(=O)c3nc(C(C)(C)C)no3)c(C)c2F)C1 ; Cl]

3-(tert-Butyl)-N-(3-fluoro-2-methyl-4-(3-(3-(methylamino)piperidin-1-yl)pyridin-4-yl)benzyl)-1,2,4-oxadiazole-5-carboxamide hydrochloride as obtained as a yellow solid from tert-butyl (1-(4-(4-((3-(tert-butyl)-1,2,4-oxadiazole-5-carboxamido)methyl)-2-fluoro-3-methylphenyl)pyridin-3-yl)piperidin-3-yl)(methyl)carbamate, following the procedure described in Example 2, step 3: 1-(tert-butyl)-N-(2-methyl-4-(3-(3-(methylamino)pyrrolidin-1-yl)pyridin-4-yl)benzyl)-1H-1,2,3-triazole-4-carboxamide hydrochloride (170 mg, crude). LCMS m/z=481.3 (M+H)+.

### 3. Synthesis of 3-(tert-butyl)-N-(3-fluoro-2-methyl-4-(3-(3-(N-methylacrylamido)piperidin-1-yl)pyridin-4-yl)benzyl)-1,2,4-oxadiazole-5-carboxamide

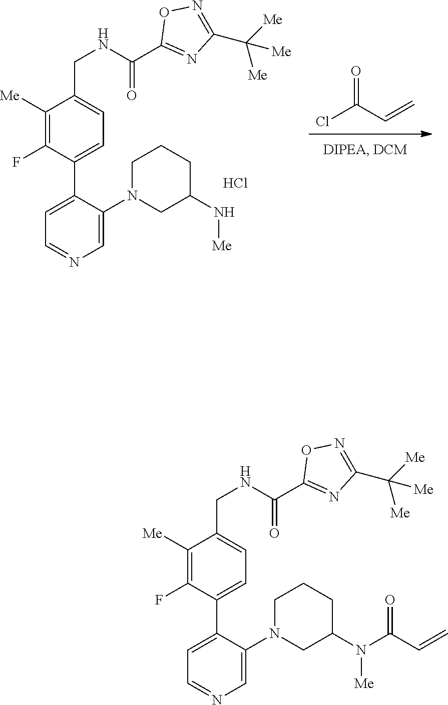

The compound was obtained from 3-(tert-butyl)-N-(3-fluoro-2-methyl-4-(3-(3-(methylamino)piperidin-1-yl)pyridin-4-yl)benzyl)-1,2,4-oxadiazole-5-carboxamide hydrochloride and acryloyl chloride following a similar procedure to that described in Example 1, step 4: 1-(tert-butyl)-N-(2-methyl-4-(3-(3-(N-methylacrylamido)piperidin-1-yl)pyridin-4-yl)benzyl)-1H-1,2,3-triazole-4-carboxamide. The crude was purified by prep-HPLC (Method A1, organic gradient 39-69%) to give 3-(tert-butyl)-N-(3-fluoro-2-methyl-4-(3-(3-(N-methylacrylamido)piperidin-1-yl)pyridin-4-yl)benzyl)-1,2,4-oxadiazole-5-carboxamide (77 mg, 43% yield) as a yellow solid. LCMS m/z=535.3 (M+H)+. ¹HNMR (400 MHz, DMSO-d₆) δ: 9.87 (s, 1H), 8.42 (s, 1H), 8.28 (s, 1H), 7.38-7.22 (m, 2H), 7.17 (d, 1H), 6.71-5.92 (m, 2H), 5.60 (t, 1H), 4.53 (s, 2H), 4.23-3.53 (m, 1H), 2.97-2.71 (m, 7H), 2.28 (s, 3H), 1.65-1.35 (m, 13H).

### Example 19: 5-(tert-butyl)-N-(3-fluoro-2-methyl-4-(3-(3-(N-methylacrylamido)piperidin-1-yl)pyridin-4-yl)benzyl)-1,2,4-oxadiazole-3-carboxamide

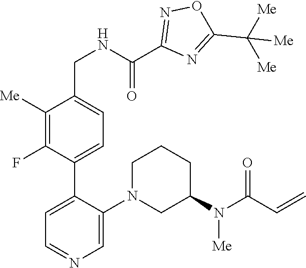 [SMILES: C=CC(=O)N(C)[C@@H]1CCCN(c2cnccc2-c2ccc(CNC(=O)c3noc(C(C)(C)C)n3)c(C)c2F)C1]

### 1. Synthesis of tert-butyl (1-(4-(4-((5-(tert-butyl)-1,2,4-oxadiazole-3-carboxamido)methyl)-2-fluoro-3-methylphenyl)pyridin-3-yl)piperidin-3-yl)(methyl)carbamate

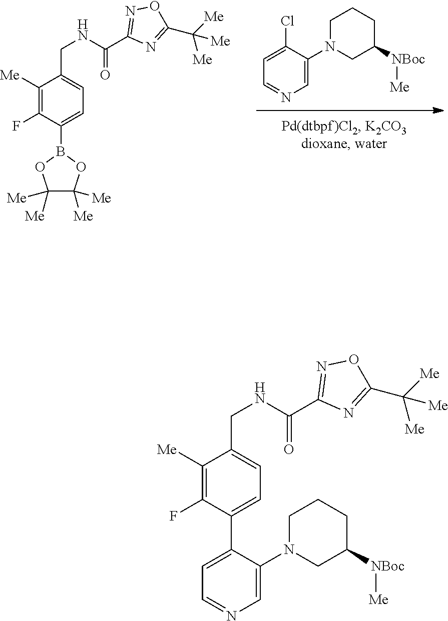 [SMILES: Cc1c(CNC(=O)c2noc(C(C)(C)C)n2)ccc(B2OC(C)(C)C(C)(C)O2)c1F ; CN(C(=O)OC(C)(C)C)[C@@H]1CCCN(c2cnccc2Cl)C1 ; Cc1c(CNC(=O)c2noc(C(C)(C)C)n2)ccc(-c2ccncc2N2CCC[C@@H](N(C)C(=O)OC(C)(C)C)C2)c1F]

The compound was obtained from Example 1, step 1: tert-butyl (1-(4-chloropyridin-3-yl)piperidin-3-yl)(methyl)carbamate (648 mg, 2.0 mmol) and Intermediate 7: 3-(tert-butyl)-N-(3-fluoro-2-methyl-4-(4,4,5,5-tetramethyl-1,3,2-dioxaborolan-2-yl)benzyl)-1,2,4-oxadiazole-5-carboxamide following a similar procedure to that described in Example 4, step 2: tert-butyl 7-(4-(4-((1-(tert-butyl)-1H-1,2,3-oxadiazole-4-carboxamido)methyl)-3-methylphenyl)pyridin-3-yl)-2,7-diazaspiro[4.4]nonane-2-carboxylate. The crude was purified by silica gel column chromatography (3:1 EtOAc:EtOH):heptane (0/100 to 100/0) to give tert-butyl (1-(4-(4-((5-(tert-butyl)-1,2,4-oxadiazole-3-carboxamido)methyl)-2-fluoro-3-methylphenyl)pyridin-3-yl)piperidin-3-yl)(methyl)carbamate (470 mg, 41% yield) as an off-white solid. LCMS m/z=581.3 (M+H)+.

### 2. Synthesis of 5-(tert-butyl)-N-(3-fluoro-2-methyl-4-(3-(3-(methylamino)piperidin-1-yl)pyridin-4-yl)benzyl)-1,2,4-oxadiazole-3-carboxamide hydrochloride

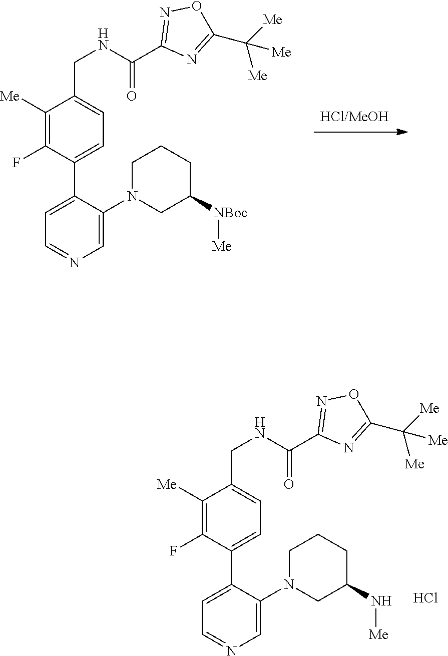 [SMILES: Cc1c(CNC(=O)c2noc(C(C)(C)C)n2)ccc(-c2ccncc2N2CCC[C@@H](N(C)C(=O)OC(C)(C)C)C2)c1F ; CN[C@@H]1CCCN(c2cnccc2-c2ccc(CNC(=O)c3noc(C(C)(C)C)n3)c(C)c2F)C1 ; Cl]

HCl/MeOH (1.25 M, 6.5 mL) was added to a solution of tert-butyl (1-(4-(4-((5-(tert-butyl)-1,2,4-oxadiazole-3-carboxamido)methyl)-2-fluoro-3-methylphenyl)pyridin-3-yl)piperidin-3-yl)(methyl)carbamate (470 mg, 809 μmol) in MeOH (2.7 mL) and the reaction was stirred for 6 h at 50° C. The cooled reaction was evaporated under reduced pressure to give 5-(tert-butyl)-N-(3-fluoro-2-methyl-4-(3-(3-(methylamino)piperidin-1-yl)pyridin-4-yl)benzyl)-1,2,4-oxadiazole-3-carboxamide hydrochloride (465 mg, crude) as a light yellow solid. LCMS m/z=481.2 (M+H)+.

### 3. Synthesis of 5-(tert-butyl)-N-(3-fluoro-2-methyl-4-(3-(3-(N-methylacrylamido)piperidin-1-yl)pyridin-4-yl)benzyl)-1,2,4-oxadiazole-3-carboxamide

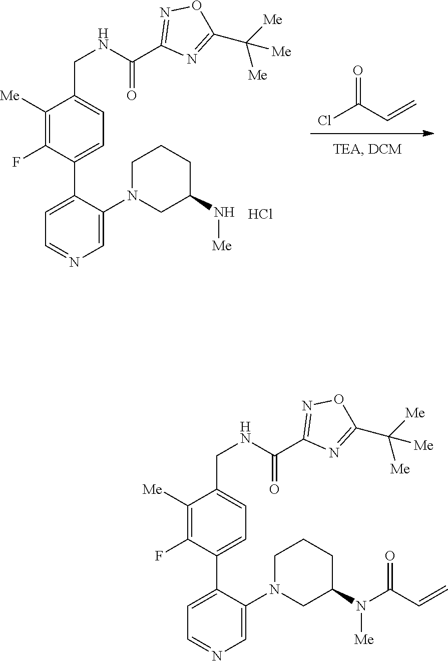

A solution of 5-(tert-butyl)-N-(3-fluoro-2-methyl-4-(3-(3-(methylamino)piperidin-1-yl)pyridin-4-yl)benzyl)-1,2,4-oxadiazole-3-carboxamide hydrochloride (150 mg, 271 μmol), DCM (2.7 mL) and TEA (123 mg, 1.21 mmol) was cooled for 5 mins in a dry ice/acetone bath. Acryloyl chloride (26 mg, 285 μmol) was added dropwise and the reaction was stirred for 15 mins. The reaction mixture was purified directly by silica gel column chromatography eluting with (3:1 EtOAc:EtOH):heptane (0/100 to 100/0) to give 5-(tert-butyl)-N-(3-fluoro-2-methyl-4-(3-(3-(N-methylacrylamido)piperidin-1-yl)pyridin-4-yl)benzyl)-1,2,4-oxadiazole-3-carboxamide (104 mg, 72% yield) as a light yellow film. LCMS m/z=535.2 (M+H)+. ¹H NMR (500 MHz, DMSO-d₆) δ: 9.58-9.39 (m, 1H), 8.48-8.37 (m, 1H), 8.27 (br d, 1H), 7.42-7.25 (m, 1H), 7.23-7.13 (m, 2H), 6.77-6.57 (m, 1H), 6.16-5.90 (m, 1H), 5.68-5.51 (m, 1H), 4.60-4.42 (m, 2H), 4.22 (br s, 1H), 3.54 (br s, 1H), 3.02-2.75 (m, 4H), 2.71 (br s, 2H), 2.28 (s, 3H), 1.80-1.49 (m, 3H), 1.43 (s, 9H), 1.31-1.22 (m, 1H).

### Example 20: 5-(tert-butyl)-N-(2-methyl-4-(3-(3-((N-methylacrylamido)methyl)pyrrolidin-1-yl)pyridin-4-yl)benzyl)-1,2,4-oxadiazole-3-carboxamide

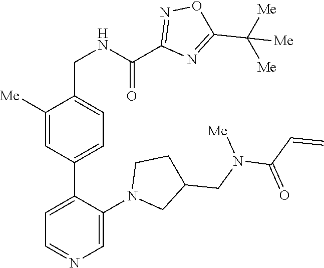 [SMILES: C=CC(=O)N(C)CC1CCN(c2cnccc2-c2ccc(CNC(=O)c3noc(C(C)(C)C)n3)c(C)c2)C1]

### 1. Synthesis of tert-butyl ((1-(4-chloropyridin-3-yl)pyrrolidin-3-yl)methyl)(methyl)carbamate

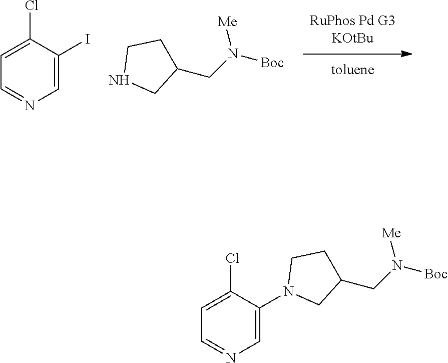

A mixture of 4-chloro-3-iodopyridine (250 mg, 1.04 mmol), tert-butyl N-methyl-N-(pyrrolidin-3-ylmethyl)carbamate (245 mg, 1.14 mmol), KOtBu (233 mg, 2.08 mmol) and RuPhos Pd G3 (87 mg, 104 μmol) in dry, degassed toluene (4 mL) was stirred at 90° C. under N₂ for 17 h. The cooled mixture was filtered through Celite®, washing through with EtOAc, and the filtrate was concentrated in vacuo. The crude product was purified by silica gel column chromatography eluting with EtOAc/heptanes (0/100 to 70/30) to give tert-butyl ((1-(4-chloropyridin-3-yl)pyrrolidin-3-yl)methyl)(methyl)carbamate (121 mg, 36% yield) as an oil. LCMS m/z=326.1 (M+H)+.

### 2. Synthesis of tert-butyl ((1-(4-(4-((5-(tert-butyl)-1,2,4-oxadiazole-3-carboxamido)methyl)-3-methylphenyl)pyridin-3-yl)pyrrolidin-3-yl)methyl)(methyl)carbamate

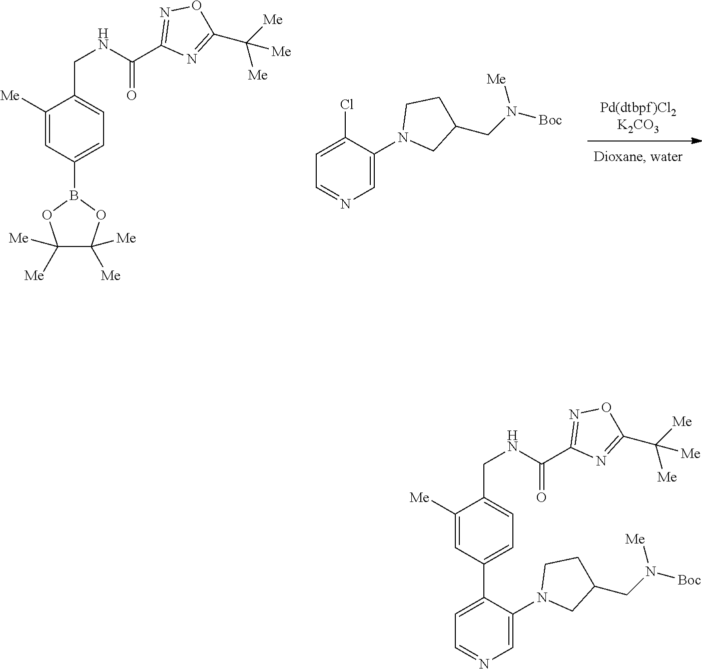 [SMILES: Cc1cc(B2OC(C)(C)C(C)(C)O2)ccc1CNC(=O)c1noc(C(C)(C)C)n1 ; CN(CC1CCN(c2cnccc2Cl)C1)C(=O)OC(C)(C)C ; Cc1cc(-c2ccncc2N2CCC(CN(C)C(=O)OC(C)(C)C)C2)ccc1CNC(=O)c1noc(C(C)(C)C)n1]

A mixture of Intermediate 5: 5-(tert-butyl)-N-(2-methyl-4-(4,4,5,5-tetramethyl-1,3,2-dioxaborolan-2-yl)benzyl)-1,2,4-oxadiazole-3-carboxamide (191 mg, 479 μmol), tert-butyl ((1-(4-chloropyridin-3-yl)pyrrolidin-3-yl)methyl)(methyl)carbamate (120 mg, 368 μmol) and K₂CO₃ (153 mg, 1.10 mmol) in dioxane (3 mL) and water (500 μL) was purged with N₂ for 5 min. Pd(dtbpf)Cl₂ (24 mg, 37 μmol) was added and the reaction was heated at 90° C. for 18 h. Additional Pd(dtbpf)Cl₂ (24 mg, 37 μmol) was added and the reaction was stirred at 90° C. for a further 24 h. The cooled reaction was diluted with water and EtOAc, filtered through Celite® washing through with EtOAc, and the layers were separated. The aqueous phase was extracted with EtOAc, the combined organic extracts dried (MgSO₄), filtered and concentrated in vacuo. The residue was purified by silica gel column chromatography eluting with EtOAc/heptanes (0/100 to 100/0) to give tert-butyl ((1-(4-(4-((5-(tert-butyl)-1,2,4-oxadiazole-3-carboxamido)methyl)-3-methylphenyl)pyridin-3-yl)pyrrolidin-3-yl)methyl)(methyl)carbamate as a brown oil (22 mg, 11% yield). LCMS m/z=563.3 (M+H)+.

### 3. Synthesis of tert-butyl ((1-(4-(4-((5-(tert-butyl)-1,2,4-oxadiazole-3-carboxamido)methyl)-3-methylphenyl)pyridin-3-yl)pyrrolidin-3-yl)methyl)(methyl)carbamate hydrochloride

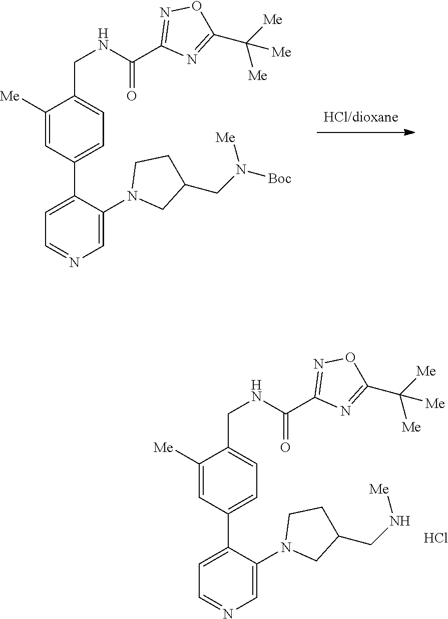 [SMILES: Cc1cc(-c2ccncc2N2CCC(CN(C)C(=O)OC(C)(C)C)C2)ccc1CNC(=O)c1noc(C(C)(C)C)n1 ; CNCC1CCN(c2cnccc2-c2ccc(CNC(=O)c3noc(C(C)(C)C)n3)c(C)c2)C1 ; Cl]

A solution of tert-butyl ((1-(4-(4-((5-(tert-butyl)-1,2,4-oxadiazole-3-carboxamido)-methyl)-3-methylphenyl)pyridin-3-yl)pyrrolidin-3-yl)methyl)(methyl)carbamate (25 mg, 43 μmol) in 4M HCl/dioxane (500 μL) was stirred at rt for 1 h, then evaporated under reduced pressure to give tert-butyl ((1-(4-(4-((5-(tert-butyl)-1,2,4-oxadiazole-3-carboxamido)methyl)-3-methylphenyl)pyridin-3-yl)pyrrolidin-3-yl)methyl)(methyl)carbamate hydrochloride (19 mg, crude) as a yellow solid. LCMS m/z=463.2 (M+H)+.

### 4. Synthesis of 5-(tert-butyl)-N-(2-methyl-4-(3-(3-((N-methylacrylamido)methyl)pyrrolidin-1-yl)pyridin-4-yl)benzyl)-1,2,4-oxadiazole-3-carboxamide

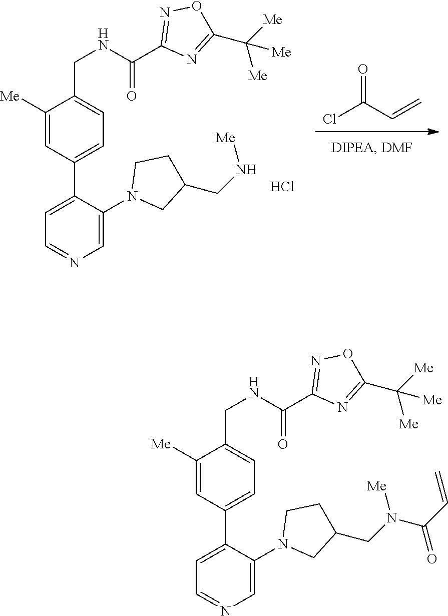

DIPEA (23 mg, 177 μmol) was added to a solution of tert-butyl ((1-(4-(4-((5-(tert-butyl)-1,2,4-oxadiazole-3-carboxamido)methyl)-3-methylphenyl)pyridin-3-yl)pyrrolidin-3-yl)methyl)(methyl)carbamate hydrochloride (19 mg, 35 μmol) in dry DMF (1 mL) at 0° C. Acryloyl chloride (6 mg, 71 μmol) was then added dropwise and the resulting mixture was stirred at 0° C. for 15 min, quenched with sat. aq. NaHCO₃ solution. The layers were separated and the organic phase was washed with brine (3 x). The combined aqueous layers were extracted with EtOAc, and the combined organic phases were dried (MgSO₄), filtered, and concentrated in vacuo. The residue was purified by preparative TLC (DCM/MeOH 96/4) to give 5-(tert-butyl)-N-(2-methyl-4-(3-(3-((N-methylacrylamido)methyl)pyrrolidin-1-yl)pyridin-4-yl)benzyl)-1,2,4-oxadiazole-3-carboxamide as an oil (2.60 mg, 13% yield). LCMS m/z=517.2 (M+H)+. ¹H NMR (500 MHz, CDCl₃) δ: 8.21-8.16 (m, 1H), 8.14-8.06 (m, 1H), 7.38-7.29 (m, 1H), 7.34-7.29 (m, 1H), 7.26-7.20 (m, 2H), 7.05-6.98 (m, 1H), 6.57-6.41 (m, 1H), 6.35-6.21 (m, 1H), 5.72-5.57 (m, 1H), 4.79-4.62 (m, 2H), 3.53-3.28 (m, 2H), 3.13-2.90 (m, 6H), 2.80-2.70 (m, 1H), 2.54-2.44 (m, 1H), 2.42-2.38 (m, 3H), 2.02-1.88 (m, 1H), 1.47 (d, 10H).

### Example 21: 5-(tert-butyl)-N-(2-methyl-4-(3-(3-((N-methylacrylamido)methyl)piperidin-1-yl)pyridin-4-yl)benzyl)-1,2,4-oxadiazole-3-carboxamide

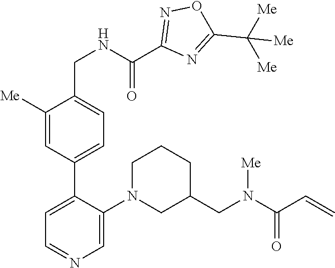 [SMILES: C=CC(=O)N(C)CC1CCCN(c2cnccc2-c2ccc(CNC(=O)c3noc(C(C)(C)C)n3)c(C)c2)C1]

### 1. Synthesis of tert-butyl ((1-(4-chloropyridin-3-yl)piperidin-3-yl)methyl)(methyl)carbamate

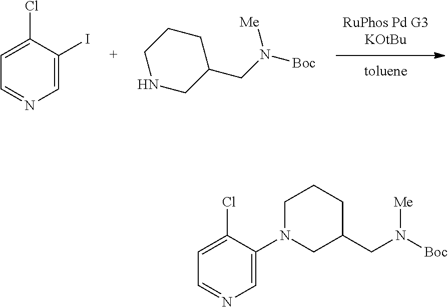 [SMILES: Clc1ccncc1I ; CN(CC1CCCNC1)C(=O)OC(C)(C)C ; CN(CC1CCCN(c2cnccc2Cl)C1)C(=O)OC(C)(C)C]

tert-Butyl ((1-(4-chloropyridin-3-yl)piperidin-3-yl)methyl)(methyl)carbamate was obtained as an oil from 4-chloro-3-iodopyridine and tert-butyl N-methyl-N-(piperidin-3-ylmethyl)carbamate, following the procedure described in Example 20, step 1: tert-butyl ((1-(4-chloropyridin-3-yl)pyrrolidin-3-yl)methyl)(methyl)carbamate. LCMS m/z=340.1 (M+H)+.

### 2. Synthesis of tert-butyl ((1-(4-(4-((5-(tert-butyl)-1,2,4-oxadiazole-3-carboxamido)methyl)-3-methylphenyl)pyridin-3-yl)piperidin-3-yl)methyl)(methyl)carbamate

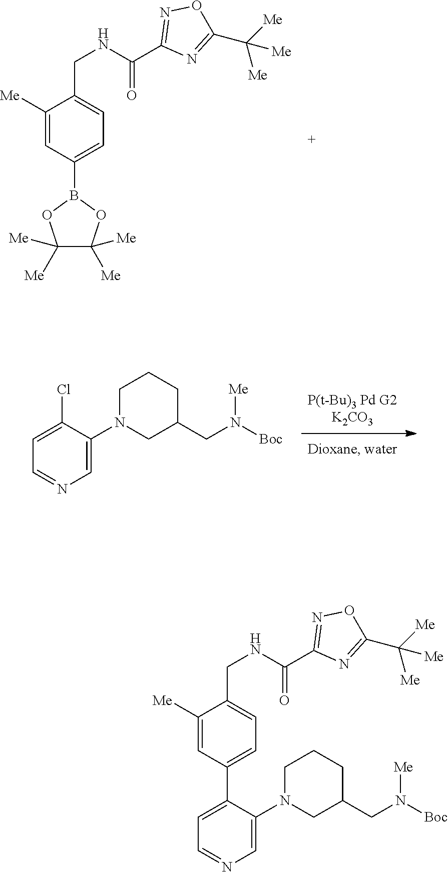 [SMILES: Cc1cc(B2OC(C)(C)C(C)(C)O2)ccc1CNC(=O)c1noc(C(C)(C)C)n1 ; CN(CC1CCCN(c2cnccc2Cl)C1)C(=O)OC(C)(C)C ; Cc1cc(-c2ccncc2N2CCCC(CN(C)C(=O)OC(C)(C)C)C2)ccc1CNC(=O)c1noc(C(C)(C)C)n1]

A mixture of Intermediate 5: 5-(tert-butyl)-N-(2-methyl-4-(4,4,5,5-tetramethyl-1,3,2-dioxaborolan-2-yl)benzyl)-1,2,4-oxadiazole-3-carboxamide (305 mg, 765 μmol), tert-butyl ((1-(4-chloropyridin-3-yl)piperidin-3-yl)methyl)(methyl)carbamate (200 mg, 588 μmol) and P(t-Bu)₃Pd G2 (30 mg, 59 μmol) in dioxane (2 mL) and water (400 μL) was purged with N₂ for 5 min. K₂CO₃ (325 mg, 2.35 mmol) was added and the reaction was heated to 90° C. for 2 days. The cooled reaction was diluted with EtOAc and water, and the layers were separated. The organic layer was washed with brine (3 x), the combined aqueous layers were extracted with EtOAc, and the combined organic extracts were dried (MgSO₄), filtered and concentrated in vacuo. The residue was purified by silica gel column chromatography eluting with EtOAc/heptanes (0/100 to 100/0) to give tert-butyl ((1-(4-(4-((5-(tert-butyl)-1,2,4-oxadiazole-3-carboxamido)methyl)-3-methylphenyl)pyridin-3-yl)piperidin-3-yl)methyl)(methyl)carbamate (180 mg, 53% yield) as an off-white solid. LCMS m/z=577.4 (M+H)+.

### 3. Synthesis of 5-(tert-butyl)-N-(2-methyl-4-(3-(3-((N-methylacrylamido)methyl)piperidin-1-yl)pyridin-4-yl)benzyl)-1,2,4-oxadiazole-3-carboxamide

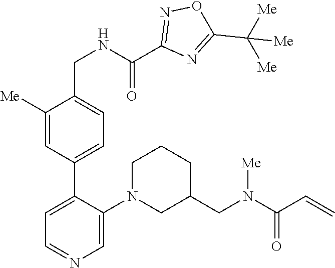 [SMILES: C=CC(=O)N(C)CC1CCCN(c2cnccc2-c2ccc(CNC(=O)c3noc(C(C)(C)C)n3)c(C)c2)C1]

5-(tert-Butyl)-N-(2-methyl-4-(3-(3-((N-methylacrylamido)methyl)piperidin-1-yl)pyridin-4-yl)benzyl)-1,2,4-oxadiazole-3-carboxamide was obtained as an oil from tert-butyl ((1-(4-(4-((5-(tert-butyl)-1,2,4-oxadiazole-3-carboxamido)methyl)-3-methylphenyl)pyridin-3-yl)piperidin-3-yl)methyl)(methyl)carbamate following the procedure described in Example 20, steps 3 to 4. LCMS m/z=531.3 (M+H)+. ¹H NMR (400 MHz, CDCl₃) δ: 8.36-8.23 (m, 1H), 7.53-7.28 (m, 2H), 7.13 (br dd, 1H), 6.46 (br d, 1H), 6.38-6.24 (m, 1H), 6.42-6.22 (m, 1H), 6.21-6.10 (m, 1H), 5.88 (br d, 1H), 5.83-5.53 (m, 1H), 4.96-4.43 (m, 2H), 3.49-2.81 (m, 4H), 2.78-2.64 (m, 3H), 2.45-2.34 (m, 3H), 2.28-1.97 (m, 1H), 1.97-1.54 (m, 3H), 1.33-1.20 (m, 6H), 1.54-1.19 (m, 6H).

### Example 22: (R)-1-(tert-butyl)-N-(2-methyl-4-(3-(3-(N-methylacrylamido)piperidin-1-yl)pyridin-4-yl)benzyl)-1H-1,2,3-triazole-4-carboxamide

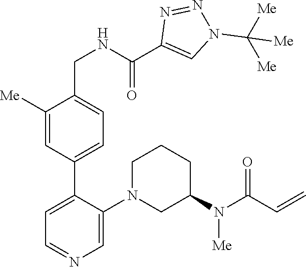 [SMILES: C=CC(=O)N(C)[C@@H]1CCCN(c2cnccc2-c2ccc(CNC(=O)c3cn(C(C)(C)C)nn3)c(C)c2)C1]

### 1. Synthesis of benzyl (R)-(1-(4-chloropyridin-3-yl)piperidin-3-yl)(methyl)carbamate

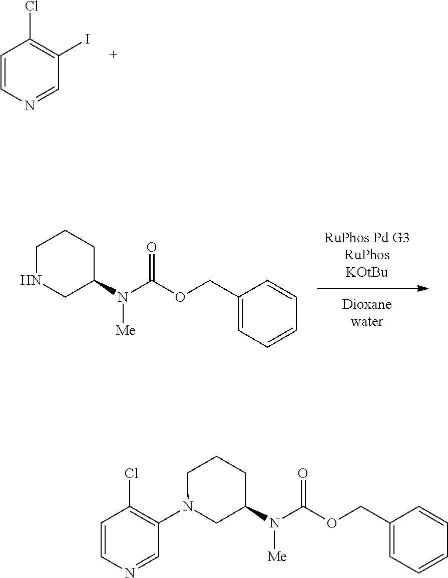 [SMILES: Clc1ccncc1I ; CN(C(=O)OCc1ccccc1)[C@@H]1CCCNC1 ; CN(C(=O)OCc1ccccc1)[C@@H]1CCCN(c2cnccc2Cl)C1]

A mixture of 4-chloro-3-iodopyridine (600 mg, 2.51 mmol), benzyl (R)-methyl(piperidin-3-yl)carbamate (571 mg, 2.30 mmol), KOtBu (352 mg, 3.14 mmol), RuPhos (98 mg, 209 μmol) and RuPhos Pd G3 (175 mg, 209 μmol) was purged with N₂. Dioxane (15 mL) was added via syringe, and the reaction was stirred at 95° C. for 3 h. The cooled mixture was diluted with EtOAc, filtered, and the filtrate was evaporated under reduced pressure. The residue was purified by silica gel column chromatography eluting with heptane/EtOAc (50/50) to give benzyl (R)-(1-(4-chloropyridin-3-yl)piperidin-3-yl)(methyl)-carbamate (480 mg, 64% yield) as a yellow sticky gum. LCMS m/z=360.2 (M+H)+.

### 2. Synthesis of benzyl (R)-(1-(4-(4-((1-(tert-butyl)-1H-1,2,3-triazole-4-carboxamido)methyl)-3-methylphenyl)pyridin-3-yl)piperidin-3-yl)(methyl)carbamate

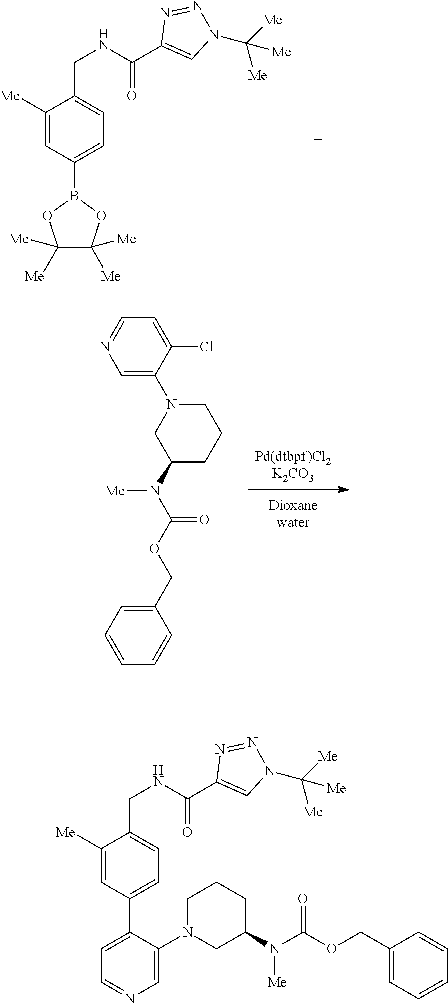

A mixture of benzyl (R)-(1-(4-chloropyridin-3-yl)piperidin-3-yl)(methyl)carbamate (480 mg, 1.33 mmol), Intermediate 1: 1-(tert-butyl)-N-(2-methyl-4-(4,4,5,5-tetramethyl-1,3,2-dioxaborolan-2-yl)benzyl)-1H-1,2,3-triazole-4-carboxamide (583 mg, 1.46 mmol), K₂CO₃ (735 mg, 5.32 mmol) and Pd(dtbpf)Cl₂ (130 mg, 200 μmol) was purged with N₂. Dioxane (6 mL) and water (1.5 mL) were added and the mixture was purged again with N₂. The reaction was stirred at rt for 5 mins and then heated at 90° C. for 3 h. The cooled mixture was diluted with EtOAc, the mixture was filtered, and the filtrate was evaporated under reduced pressure. The residue was purified by silica gel column chromatography eluting with EtOAc/MeOH (100/1) to give benzyl (R)-(1-(4-(4-((1-(tert-butyl)-1H-1,2,3-triazole-4-carboxamido)methyl)-3-methylphenyl)pyridin-3-yl)piperidin-3-yl)(methyl)carbamate (280 mg, 32% yield) as a viscous yellow gum. LCMS m/z=596.4 (M+H)+.

### 3. Synthesis of (R)-1-(tert-butyl)-N-(2-methyl-4-(3-(3-(methylamino)piperidin-1-yl)pyridin-4-yl)benzyl)-1H-1,2,3-triazole-4-carboxamide

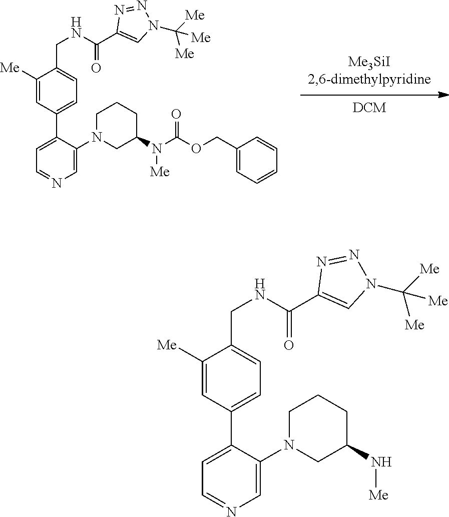

A solution of benzyl (R)-(1-(4-(4-((1-(tert-butyl)-1H-1,2,3-triazole-4-carboxamido)methyl)-3-methylphenyl)pyridin-3-yl)piperidin-3-yl)(methyl)carbamate (240 mg, 403 μmol) in DCM (5 mL) and 2,6-dimethylpyridine (6.48 g, 60.4 mmol) was cooled in an ice bath. (Me)₃SiI (3.63 g, 18.1 mmol) was added dropwise via syringe, and the reaction was allowed to warm to rt. 1M HCl (50 mL) was added dropwise very cautiously. EtOAc (80 mL) was added and the biphasic system was stirred at rt for 1 h. The phases were separated, and the aqueous phase basified by adding K₂CO₃ (solid) very cautiously, and then was extracted with EtOAc (2×). These organic extracts were combined, dried, filtered and the filtrate was evaporated under reduced pressure. The residue was purified on an SCX column eluting with TEA-MeOH to give (R)-1-(tert-butyl)-N-(2-methyl-4-(3-(3-(methylamino)piperidin-1-yl)pyridin-4-yl)benzyl)-1H-1,2,3-triazole-4-carboxamide (95 mg, 46% yield) as a yellow gum. LCMS m/z=462.3 (M+H)+.

### 4. Synthesis of (R)-1-(tert-butyl)-N-(2-methyl-4-(3-(3-(N-methylacrylamido)piperidin-1-yl)pyridin-4-yl)benzyl)-1H-1, 2,3-triazole-4-carboxamide

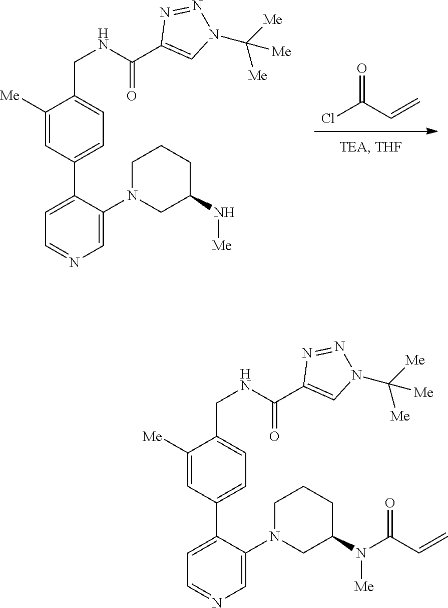

To a solution of (R)-1-(tert-butyl)-N-(2-methyl-4-(3-(3-(methylamino)piperidin-1-yl)pyridin-4-yl)benzyl)-1H-1,2,3-triazole-4-carboxamide (90 mg, 195 μmol) in THF (3 mL) was added TEA (39 mg, 390 μmol), followed by acryloyl chloride (35 mg, 390 μmol) and the reaction was stirred at rt for 1 h. The mixture was concentrated in vacuo and the crude was purified by prep HPLC (Method A2, organic gradient: 10-95%) to give (R)-1-(tert-butyl)-N-(2-methyl-4-(3-(3-(N-methylacrylamido)piperidin-1-yl)pyridin-4-yl)benzyl)-1H-1,2,3-triazole-4-carboxamide (33 mg, 31% yield) as a white solid. LCMS m/z=516.3 (M+H)+. ¹H NMR (500 MHz, CDCl₃) δ: 8.37-8.25 (m, 2H), 8.20 (s, 1H), 7.67 (br s, 1H), 7.53-7.37 (m, 3H), 7.21-7.10 (m, 1H), 6.57-6.47 (m, 0.5 H), 6.31-6.14 (m, 1H), 5.99-5.90 (m, 0.5 H), 5.68-5.56 (m, 1H), 4.77-4.49 (m, 2H), 3.68 (br s, 1H), 3.24-3.11 (br s, 1H), 3.02 (br s, 1H), 2.96-2.78 (m, 3H), 2.77-2.58 (m, 2H), 2.42 (s, 3H), 1.81 (br s, 2H), 1.70 (s, 9H), 1.61 (br s, 2H).

### Intermediate 8: tert-butyl 3-(4-(4-(aminomethyl)-3-methylphenyl)pyridin-3-yl)piperidine-1-carboxylate

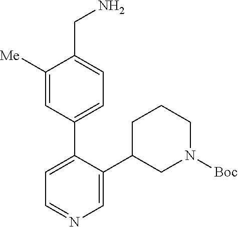 [SMILES: Cc1cc(-c2ccncc2C2CCCN(C(=O)OC(C)(C)C)C2)ccc1CN]

### 1. Synthesis of 4-(3-bromopyridin-4-yl)-2-methylbenzonitrile

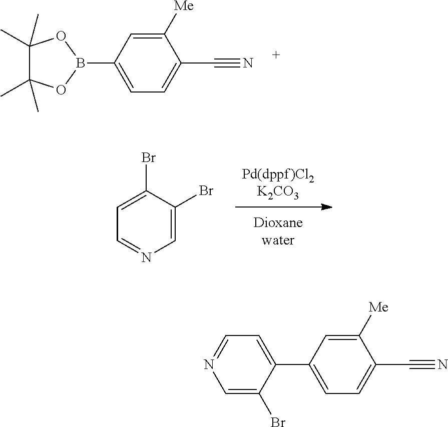 [SMILES: Cc1cc(B2OC(C)(C)C(C)(C)O2)ccc1C#N ; Brc1ccncc1Br ; Cc1cc(-c2ccncc2Br)ccc1C#N]

2-Methyl-4-(4,4,5,5-tetramethyl-1,3,2-dioxaborolan-2-yl)benzonitrile (5.0 g, 20.6 mmol) was added to a solution of 3,4-dibromopyridine (12.0 g, 50.7 mmol), Pd(dppf)Cl₂ (150 mg, 205 μmol) and K₂CO₃ (9.01 g, 65.2 mmol) in dioxane (100 mL) and water (50 mL) and the reaction mixture was degassed under N₂. The reaction was stirred at 45° C. for 18 h. Additional Pd(dppf)Cl₂ (320 mg, 437 μmol) was added and the reaction stirred at 50° C. for a further 3 h.

The cooled reaction was diluted with water (100 mL) and sat. aq. NaHCO₃ (100 mL) and the mixture was extracted with EtOAc (2×200 mL). The combined organic layers were dried over Na₂SO₄, filtered, and the filtrate evaporated under reduced pressure. The residual brown oil was purified by silica gel column chromatography eluting with heptanes/EtOAc (100/0 to 1/1). The resulting white solid was further purified by silica gel column chromatography eluting with DCM to give 4-(3-bromopyridin-4-yl)-2-methylbenzonitrile as a white solid (2.25 g, 40% yield). LCMS m/z=274.9 (M+H)+. ¹H NMR (500 MHz, DMSO-d₆) δ: 8.90 (s, 1H), 8.68-8.62 (1H), 7.94 (d, 1H), 7.61-7.58 (m, 2H), 7.50 (d, 1H), 2.56 (s, 3H).

### 2. Synthesis of tert-butyl 4′-(4-cyano-3-methylphenyl)-5,6-dihydro-[3,3′-bipyridine]-1(2H)-carboxylate

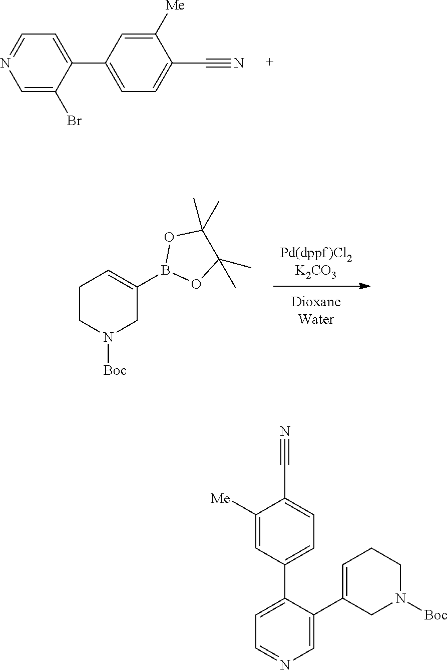 [SMILES: Cc1cc(-c2ccncc2Br)ccc1C#N ; CC(C)(C)OC(=O)N1CCC=C(B2OC(C)(C)C(C)(C)O2)C1 ; Cc1cc(-c2ccncc2C2=CCCN(C(=O)OC(C)(C)C)C2)ccc1C#N]

To 4-(3-bromopyridin-4-yl)-2-methylbenzonitrile (500 mg, 1.83 mmol) was added tert-butyl 5-(4,4,5,5-tetramethyl-1,3,2-dioxaborolan-2-yl)-3,6-dihydropyridine-1(2H)-carboxylate (1.0 g, 3.23 mmol), Pd(dppf)Cl₂ (50 mg, 68 μmol), K₂CO₃ (750 mg, 5.43 mmol), dioxane (5 mL), and water (3 mL). The reaction vessel was sealed and heated at 70° C. for 3 h. The cooled mixture was partitioned between EtOAc and water and the layers were separated. The aqueous layer was extracted with EtOAc and the combined organic extracts evaporated under reduced pressure. The crude was purified by silica gel column chromatography eluting with EtOAc/heptanes (1/1) to give tert-butyl 4′-(4-cyano-3-methylphenyl)-5,6-dihydro-[3,3′-bipyridine]-1(2H)-carboxylate (665 mg, crude), which was carried forward without further purification. LCMS m/z=376.2 (M+H)+. ¹H NMR (500 MHz, DMSO-d₆) δ: 8.63 (d, 1H), 8.50 (s, 1H), 7.97-7.75 (m, 1H), 7.70-7.44 (m, 2H), 7.42 (d, 1H), 5.99 (br s, 1H), 3.71-3.48 (m, 2H), 3.71-3.48 (m, 2H), 3.36 (s, 2H), 2.52 (s, 3H), 2.30-2.05 (m, 2H), 1.22 (br d, 9H).

### 3. Synthesis of tert-butyl 3-(4-(4-(aminomethyl)-3-methylphenyl)pyridin-3-yl)piperidine-1-carboxylate

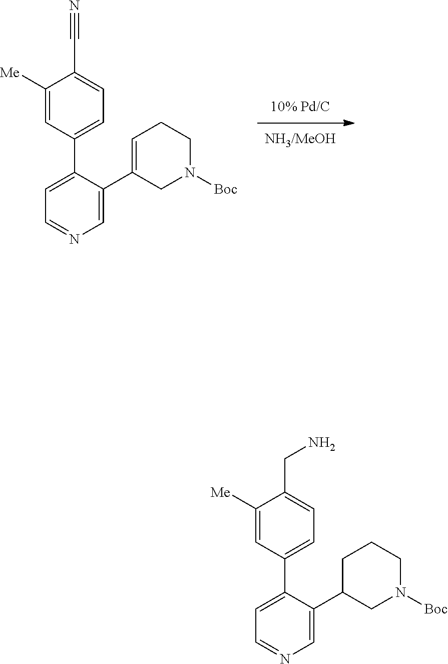 [SMILES: Cc1cc(-c2ccncc2C2=CCCN(C(=O)OC(C)(C)C)C2)ccc1C#N ; Cc1cc(-c2ccncc2C2CCCN(C(=O)OC(C)(C)C)C2)ccc1CN]

A solution of tert-butyl 4′-(4-cyano-3-methylphenyl)-5,6-dihydro-[3,3′-bipyridine]-1(2H)-carboxylate (665 mg from previous reaction) in 7.0 M NH₃/MeOH (6 mL) was added to a vial containing 10% Pd/C (700 mg, 658 μmol, 10% purity) and the vial was sealed. The vial was purged with N₂, then charged with H₂ to 100 psi and stirred at rt for 18 h. The mixture was filtered through Celite®, rinsing through with EtOH (20 mL), and the filtrate was concentrated in vacuo. The resulting oil was purified by silica gel column chromatography eluting with diisopropylamine/EtOAc (0/100 to 5/95) to give tert-butyl 4′-(4-cyano-3-methylphenyl)-5,6-dihydro-[3,3′-bipyridine]-1(2H)-carboxylate as a light yellow oil. LCMS m/z=382.2 (M+H)+. ¹H NMR (500 MHz, DMSO-d₆) δ: 8.67 (s, 1H), 8.45 (d, 1H), 7.48 (d, 1H), 7.19 (d, 1H), 7.14 (br d, 2H), 3.98-3.86 (m, 1H), 378-3.69 (m, 2H), 3.15-3.04 (m, 1H), 3.04-2.88 (m, 1H), 2.88-2.62 (m, 2H), 2.32 (s, 3H), 1.97-1.70 (m, 3H), 1.70-1.58 (m, 1H), 1.52-1.04 (m, 9H).

### Intermediate 9: tert-butyl 4-(4-(4-(aminomethyl)-3-methylphenyl)pyridin-3-yl)piperidine-1-carboxylate

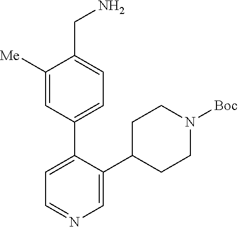 [SMILES: Cc1cc(-c2ccncc2C2CCN(C(=O)OC(C)(C)C)CC2)ccc1CN]

### 1. Synthesis of tert-butyl 4-(4-cyano-3-methylphenyl)-3′,6′-dihydro-[3,4′-bipyridine]-1′(2′H)-carboxylate

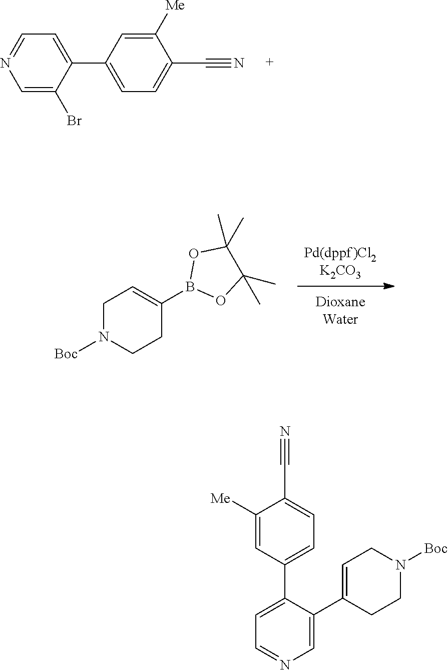 [SMILES: Cc1cc(-c2ccncc2Br)ccc1C#N ; CC(C)(C)OC(=O)N1CC=C(B2OC(C)(C)C(C)(C)O2)CC1 ; Cc1cc(-c2ccncc2C2=CCN(C(=O)OC(C)(C)C)CC2)ccc1C#N]

tert-Butyl 4-(4-cyano-3-methylphenyl)-3′,6′-dihydro-[3,4′-bipyridine]-1′(2′H)-carboxylate was obtained as an oil from 4-(3-bromopyridin-4-yl)-2-methylbenzonitrile and tert-butyl 4-(4,4,5,5-tetramethyl-1,3,2-dioxaborolan-2-yl)-3,6-dihydropyridine-1(2H)-carboxylate following the procedure described in Intermediate 8, step 1: 4-(3-bromopyridin-4-yl)-2-methylbenzonitrile (880 mg, 96% yield). LCMS m/z=376.2 (M+H)+. ¹H NMR (500 MHz, DMSO-d₆) δ: 8.60 (d, 1H), 8.50 (s, 1H), 7.84 (s, 1H), 7.59-7.52 (m, 1H), 7.47-7.41 (m, 1H), 7.41-7.35 (m, 1H), 5.78 (br s, 1H), 3.93 (s, 2H), 3.31-3.22 (m, 2H), 2.53 (s, 3H), 1.96-1.78 (m, 2H), 1.40 (br s, 9H).

### 2. Synthesis of tert-butyl 4-(4-(4-(aminomethyl)-3-methylphenyl)pyridin-3-yl)piperidine-]-carboxylate

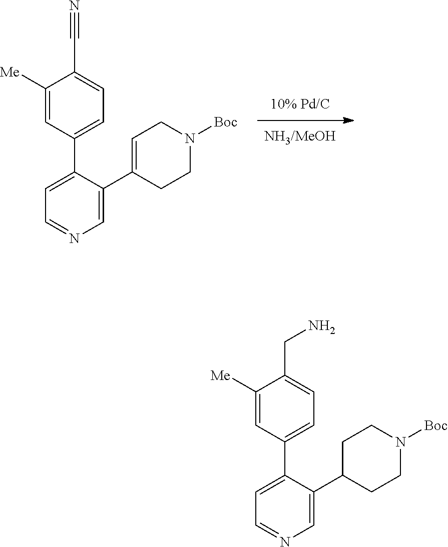 [SMILES: Cc1cc(-c2ccncc2C2=CCN(C(=O)OC(C)(C)C)CC2)ccc1C#N ; Cc1cc(-c2ccncc2C2CCN(C(=O)OC(C)(C)C)CC2)ccc1CN]

A solution of tert-butyl 4-(4-cyano-3-methylphenyl)-3′,6′-dihydro-[3,4′-bipyridine]-1′(2′H)-carboxylate (880 mg, 2.34 mmol) in 7.0 M NH3/MeOH (7 mL) was added to a vial containing 10% Pd/C (500 mg, 470 μmol, 10% purity) and the vial was sealed. The vial was purged with N₂, then was charged with H₂ to 100 psi and stirred at rt overnight. Additional Pd/C (200 mg, 10%) was added, and the reaction was stirred under an atmosphere of H₂ for a further 72 h. The mixture was filtered through Celite®, rinsing through with EtOH (10 mL), and the filtrate was concentrated in vacuo. The resulting oil was purified by silica gel column chromatography (eluting with 100% EtOAc to 13% EtOH in EtOAc with 1% diethylamine) to give tert-butyl 4-(4-(4-(aminomethyl)-3-methylphenyl)pyridin-3-yl)piperidine-1-carboxylate as a gummy solid. ¹H NMR (500 MHz, DMSO-d₆) δ: 8.60 (s, 1H), 8.45-8.36 (m, 1H), 7.48 (d, 2H), 7.18-7.02 (m, 2H), 4.15-3.90 (m, 2H), 3.76 (s, 4H), 2.89-2.75 (m, 1H), 2.32 (s, 3H), 1.97-1.77 (m, 2H), 1.72-1.56 (m, 2H), 1.41 (s, 9H).

### Intermediate 10: tert-butyl (1-(4-(4-(aminomethyl)-3-methylphenyl)pyridin-3-yl)piperidin-3-yl)(methyl)carbamate

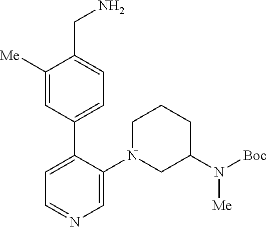 [SMILES: Cc1cc(-c2ccncc2N2CCCC(N(C)C(=O)OC(C)(C)C)C2)ccc1CN]

### 1. Synthesis of 4-(3-chloropyridin-4-yl)-2-methylbenzonitrile

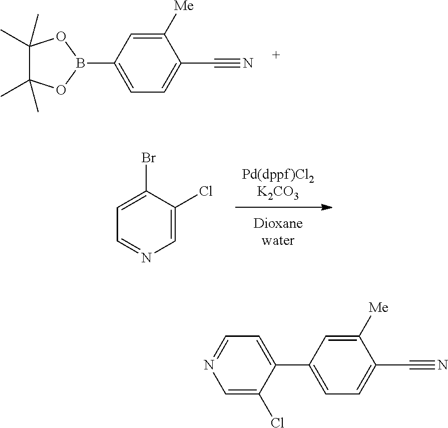 [SMILES: Cc1cc(B2OC(C)(C)C(C)(C)O2)ccc1C#N ; Clc1cnccc1Br ; Cc1cc(-c2ccncc2Cl)ccc1C#N]

A mixture of 4-bromo-3-chloropyridine (9.50 g, 49.4 mmol), 2-methyl-4-(4,4,5,5-tetramethyl-1,3,2-dioxaborolan-2-yl)benzonitrile (9.70 g, 39.9 mmol), Pd(dppf)Cl₂ (125 mg, 171 μmol) and K₂CO₃ (17 g, 123 mmol) in dioxane (10 mL) and water (5 mL) was degassed with N₂ and the reaction was stirred at 65° C. for 3 h. The cooled reaction was washed with brine (150 mL) and the layers were separated. The aqueous phase was extracted with EtOAc (150 mL), and the combined organic layers were dried over Na₂SO₄, filtered through Celite®, rinsing with EtOAc (100 mL), and evaporated under reduced pressure. The material was purified by silica gel column chromatography eluting with heptane to EtOAc to give 4-(3-chloropyridin-4-yl)-2-methylbenzonitrile as a white solid (10.1 g, 100% yield). ¹H NMR (500 MHz, DMSO-d₆) δ: 8.79 (s, 1H), 8.64 (d, 1H), 7.94 (d, 1H), 7.64 (s, 1H), 7.58-7.49 (m, 2H), 2.56 (3H).

### 2. Synthesis of tert-butyl (1-(4-(4-cyano-3-methylphenyl)pyridin-3-yl)piperidin-3-yl)(methyl)carbamate

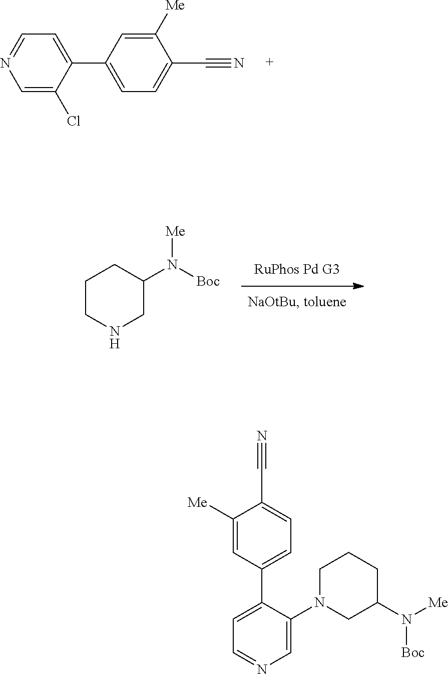 [SMILES: Cc1cc(-c2ccncc2Cl)ccc1C#N ; CN(C(=O)OC(C)(C)C)C1CCCNC1 ; Cc1cc(-c2ccncc2N2CCCC(N(C)C(=O)OC(C)(C)C)C2)ccc1C#N]

A mixture of 4-(3-chloropyridin-4-yl)-2-methylbenzonitrile (5.0 g, 21.9 mmol), RuPhos Pd G3 (750 mg, 897 μmol), and tert-butyl N-methyl-N-(3-piperidyl)carbamate (5.01 g, 23.4 mmol) in toluene (100 mL) was degassed with N₂. NaOtBu (5.0 g, 52.0 mmol) was added and the reaction was stirred at 100° C. for 3 h. The cooled reaction was diluted with MTBE (75 mL) and the mixture was stirred for 1 h, then filtered through Celite®. The filtrate was evaporated under reduced pressure and the crude was purified by silica gel column chromatography eluting with heptanes to EtOAc to give tert-butyl (1-(4-(4-cyano-3-methylphenyl)pyridin-3-yl)piperidin-3-yl)(methyl)carbamate as a yellow oil (5.77 g, 65% yield). ¹H NMR (500 MHz, DMSO-d₆) δ: 8.40 (br s, 1H), 8.31 (br d, 1H), 7.86 (br d, 1H), 7.75 (s, 1H), 7.70-7.55 (m, 1H), 7.25 (d, 1H), 3.91-3.58 (m, 3H), 3.08-2.92 (m, 2H), 2.91-2.76 (m, 2H), 2.61 (s, 3H), 2.54 (s, 3H), 1.72-1.55 (m, 2H), 1.36 (s, 9H).

### 3. Synthesis of tert-butyl (1-(4-(4-cyano-3-methylphenyl)pyridin-3-yl)piperidin-3-yl)(methyl)carbamate

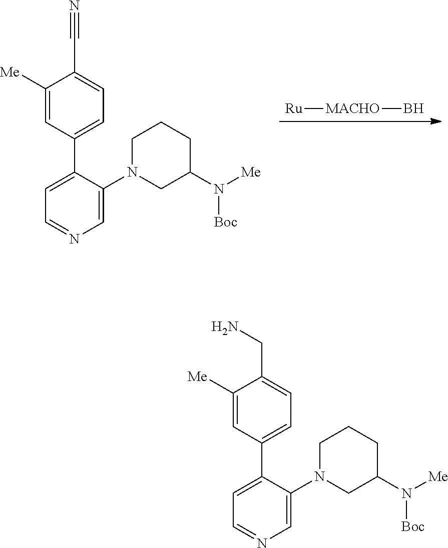

A solution of tert-butyl (1-(4-(4-cyano-3-methylphenyl)pyridin-3-yl)piperidin-3-yl)(methyl)carbamate (5.77 g, 14.2 mmol) in IPA (75 mL) was divided between 15 reaction vials. Carbonylhydrido(tetrahydroborato)[bis(2-diphenylphosphinoethyl)amino]ruthenium(II) (Ru-MACHO-BH, 15 mg, 25.5 μmol) was added to each and the reactions were degassed with N₂. The reactions were charged to 95 psi H₂ and heated at 100° C. for 3 h. The cooled reactions were combined and concentrated in vacuo to give a dark brown oil which was purified by silica gel column chromatography eluting with EtOAc/EtOH (100/0 to 75/25) to give tert-butyl (1-(4-(4-cyano-3-methylphenyl)pyridin-3-yl)piperidin-3-yl)(methyl)carbamate as an oil (4.88 g, 84% yield). ¹H NMR (500 MHz, DMSO-d₆) δ: 8.33 (s, 1H), 8.24 (d, 1H), 7.53-7.38 (m, 3H), 7.18 (d, 1H), 3.74 (s, 2H), 3.07-2.89 (m, 2H), 2.85-2.72 (m, 2H), 2.66 (s, 3H), 2.49-2.37 (m, 1H), 2.32 (s, 3H), 1.98-1.71 (m, 2H), 1.71-1.46 (m, 2H), 1.37 (s, 9H).

### Example 23: N-(4-(3-(1-acryloylpiperidin-3-yl)pyridin-4-yl)-2-methylbenzyl)-1-(tert-butyl)-1H-1,2,3-triazole-4-carboxamide

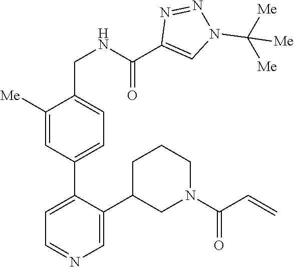 [SMILES: C=CC(=O)N1CCCC(c2cnccc2-c2ccc(CNC(=O)c3cn(C(C)(C)C)nn3)c(C)c2)C1]

### 1. Synthesis of tert-butyl 3-(4-(4-((1-(tert-butyl)-1H-1,2,3-triazole-4-carboxamido)methyl)-3-methylphenyl)pyridin-3-yl)piperidine-1-carboxylate

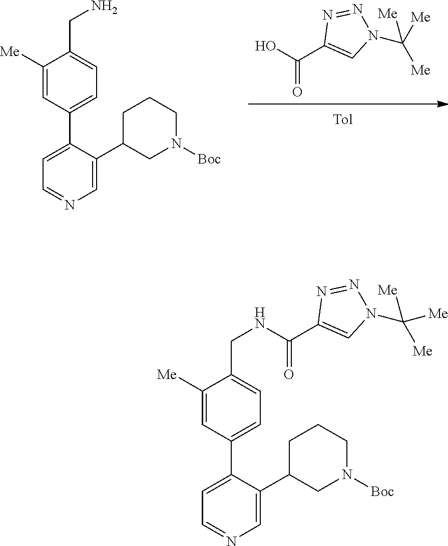 [SMILES: Cc1cc(-c2ccncc2C2CCCN(C(=O)OC(C)(C)C)C2)ccc1CN ; CC(C)(C)n1cc(C(=O)O)nn1 ; Cc1cc(-c2ccncc2C2CCCN(C(=O)OC(C)(C)C)C2)ccc1CNC(=O)c1cn(C(C)(C)C)nn1]

Tetramethyl silicate (110 mg, 723 μmol) was added to a solution of Intermediate 8: tert-butyl 3-(4-(4-(aminomethyl)-3-methylphenyl)pyridin-3-yl)piperidine-1-carboxylate (95 mg, 249 μmol), and 1-tert-butyltriazole-4-carboxylic acid (54 mg, 319 μmol) in toluene (2 mL). The reaction vial was sealed and heated at 100° C. for 18 h. The cooled reaction was diluted with DCM (5 mL) and purified directly by silica gel column chromatography eluting with heptanes to EtOAc to give tert-butyl 3-(4-(4-((1-(tert-butyl)-1H-1,2,3-triazole-4-carboxamido)methyl)-3-methylphenyl)pyridin-3-yl)piperidine-1-carboxylate (79 mg, 60% yield) as a crystalline solid. LCMS m/z=533.3 (M+H)+. ¹H NMR (500 MHz, DMSO-d₆) δ: 9.00 (br s, 1H), 8.75-8.65 (m, 2H), 8.45 (d, 1H), 7.40-7.25 (m, 2H), 7.20 (br d, 2H), 4.55-4.43 (m, 2H), 3.98-3.55 (m, 4H), 3.05-2.89 (m, 1H), 2.83-2.66 (m, 2H), 2.39 (s, 3H), 1.93-1.71 (m, 2H), 1.65 (s, 9H), 1.44-1.09 (m, 9H).

### 2. Synthesis of 1-(tert-butyl)-N-(2-methyl-4-(3-(piperidin-3-yl)pyridin-4-yl)benzyl)-1H-1,2,3-triazole-4-carboxamide hydrochloride

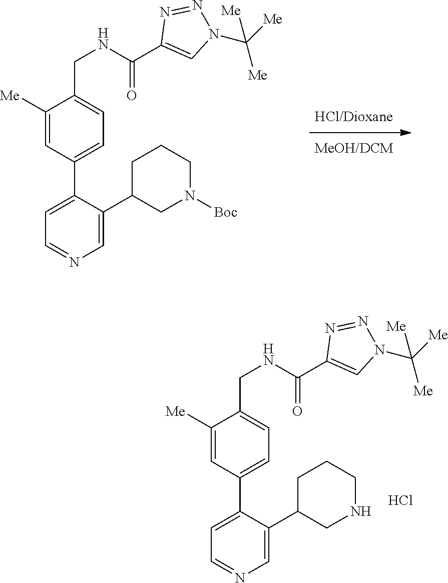 [SMILES: Cc1cc(-c2ccncc2C2CCCN(C(=O)OC(C)(C)C)C2)ccc1CNC(=O)c1cn(C(C)(C)C)nn1 ; Cc1cc(-c2ccncc2C2CCCNC2)ccc1CNC(=O)c1cn(C(C)(C)C)nn1 ; Cl]

To tert-butyl 3-(4-(4-((1-(tert-butyl)-1H-1,2,3-triazole-4-carboxamido)methyl)-3-methylphenyl)pyridin-3-yl)piperidine-1-carboxylate (79 mg, 148 μmol) was added DCM (2 mL), MeOH (2 mL), and HCl (4 M, 1 mL) and the clear solution was left to stand for 18 h at rt. The mixture was concentrated to a low volume, diluted with EtOAc (10 mL) and evaporated under reduced pressure to give 1-(tert-butyl)-N-(2-methyl-4-(3-(piperidin-3-yl)pyridin-4-yl)benzyl)-1H-1,2,3-triazole-4-carboxamide hydrochloride as an off white solid, (80 mg, crude). LCMS m/z=433.2 (M+H)+.

### 3. Synthesis of N-(4-(3-(1-acryloylpiperidin-3-yl)pyridin-4-yl)-2-methylbenzyl)-1-(tert-butyl)-1H-1,2,3-triazole-4-carboxamide

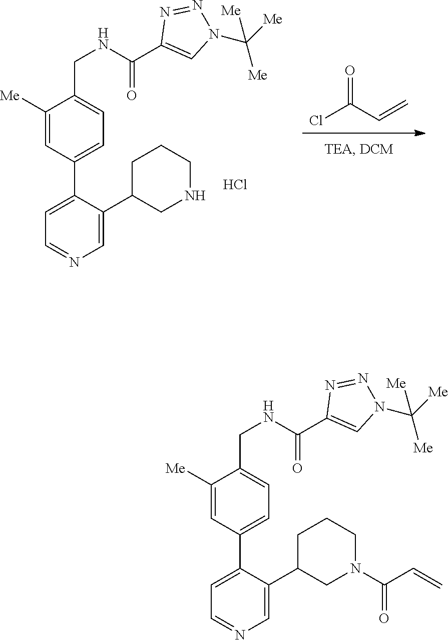

A solution of 1-(tert-butyl)-N-(2-methyl-4-(3-(piperidin-3-yl)pyridin-4-yl)benzyl)-1H-1,2,3-triazole-4-carboxamide hydrochloride (80 mg, 158 μmol) and TEA (110 μL, 791 μmol) in DCM (2 mL) was cooled to −70° C. Acryloyl chloride (13 μL, 158 μmol) was added and the reaction was stirred for 15 min. Sat. NaHCO₃ (5 mL) was added and the reaction was allowed to warm to rt. DCM (5 mL) was added, the phases were separated, and the aqueous layer was extracted with DCM (10 mL). The combined organic layers were dried over Na₂SO₄, filtered, and evaporated under reduced pressure. The crude product was dissolved in DMSO (2.3 mL), filtered through a 0.2 μm syringe filter, and purified by prep HPLC (Method C, organic gradient: 5-55%). LCMS m/z=487.1 (M+H)+. ¹H NMR (500 MHz, DMSO-d₆) δ: 8.98 (br s, 1H), 8.77-8.66 (m, 2H), 8.51-8.39 (m, 1H), 7.37-7.24 (m, 1H), 7.24-7.11 (m, 3H), 6.87-6.37 (m, 1H), 6.11-5.89 (m, 1H), 5.69-5.47 (m, 1H), 4.56-4.37 (m, 3H), 4.11-3.96 (m, 1H), 3.24-3.05 (m, 1H), 2.83-2.69 (m, 1H), 2.67-2.58 (m, 1H), 2.43-2.27 (m, 3H), 2.01-1.67 (m, 3H), 1.64 (s, 9H), 1.36-1.13 (m, 1H).

### Examples 24 and 25: (R)-N-(4-(3-(1-acryloylpiperidin-3-yl)pyridin-4-yl)-2-methylbenzyl)-1-(tert-butyl)-1H-1,2,3-triazole-4-carboxamide and (S)—N-(4-(3-(1-acryloylpiperidin-3-yl)pyridin-4-yl)-2-methylbenzyl)-1-(tert-butyl)-1H-1,2,3-triazole-4-carboxamide

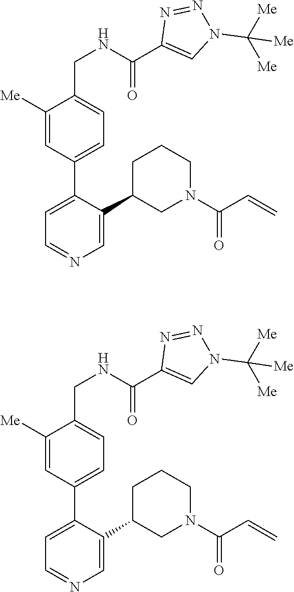 [SMILES: C=CC(=O)N1CCC[C@H](c2cnccc2-c2ccc(CNC(=O)c3cn(C(C)(C)C)nn3)c(C)c2)C1 ; C=CC(=O)N1CCC[C@@H](c2cnccc2-c2ccc(CNC(=O)c3cn(C(C)(C)C)nn3)c(C)c2)C1]

(stereochemistry arbitrarily assigned)

N-(4-(3-(1-acryloylpiperidin-3-yl)pyridin-4-yl)-2-methylbenzyl)-1-(tert-butyl)-1H-1,2,3-triazole-4-carboxamide (20 mg, 41 μmol) from Example 23 was separated by SFC (Method D) to provide (R)-N-(4-(3-(1-acryloylpiperidin-3-yl)pyridin-4-yl)-2-methylbenzyl)-1-(tert-butyl)-1H-1,2,3-triazole-4-carboxamide and (S)—N-(4-(3-(1-acryloylpiperidin-3-yl)pyridin-4-yl)-2-methylbenzyl)-1-(tert-butyl)-1H-1,2,3-triazole-4-carboxamide. The first peak (Example 24) eluted at Rt=2.91 min (7.8 mg) and the second peak (Example 25) eluted at Rt 3.36 min (7.5 mg).

### Example 26: N-(4-(3-(1-acryloylpiperidin-3-yl)pyridin-4-yl)-2-methylbenzyl)-5-(1-methylcyclopropyl)-1,2,4-oxadiazole-3-carboxamide

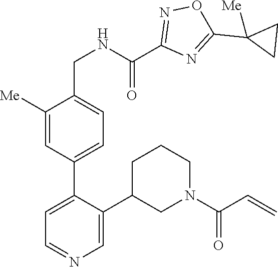 [SMILES: C=CC(=O)N1CCCC(c2cnccc2-c2ccc(CNC(=O)c3noc(C4(C)CC4)n3)c(C)c2)C1]

### 1. Synthesis of tert-butyl 3-(4-(3-methyl-4-((5-(1-methylcyclopropyl)-1,2,4-oxadiazole-3-carboxamido)methyl)phenyl)pyridin-3-yl)piperidine-1-carboxylate

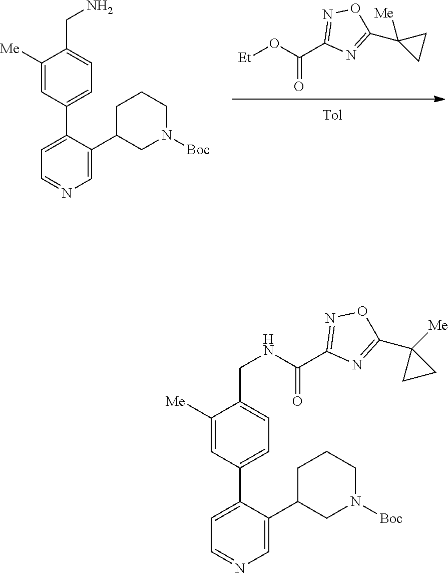 [SMILES: Cc1cc(-c2ccncc2C2CCCN(C(=O)OC(C)(C)C)C2)ccc1CN ; CCOC(=O)c1noc(C2(C)CC2)n1 ; Cc1cc(-c2ccncc2C2CCCN(C(=O)OC(C)(C)C)C2)ccc1CNC(=O)c1noc(C2(C)CC2)n1]

A mixture of Intermediate 8: tert-butyl 3-(4-(4-(aminomethyl)-3-methylphenyl)pyridin-3-yl)piperidine-1-carboxylate (115 mg, 301 μmol), ethyl 5-(1-methylcyclopropyl)-1,2,4-oxadiazole-3-carboxylate (150 mg, 765 μmol) and tetramethyl silicate (250 mg, 1.64 mmol) in toluene (2 mL) was sealed in a vial and heated at 120° C. for 18 h. The cooled reaction mixture was purified directly by silica gel column chromatography eluting with heptanes to EtOAc to give tert-butyl 3-(4-(3-methyl-4-((5-(1-methylcyclopropyl)-1,2,4-oxadiazole-3-carboxamido)methyl)phenyl)pyridin-3-yl)piperidine-1-carboxylate (40 mg, 25% yield). LCMS m/z=532.2 (M+H)+.

### 2. Synthesis of N-(4-(3-(1-acryloylpiperidin-3-yl)pyridin-4-yl)-2-methylbenzyl)-5-(1-methylcyclopropyl)-1,2,4-oxadiazole-3-carboxamide

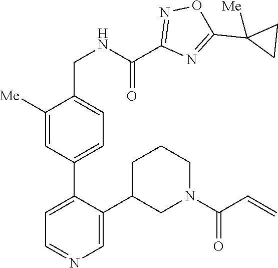 [SMILES: C=CC(=O)N1CCCC(c2cnccc2-c2ccc(CNC(=O)c3noc(C4(C)CC4)n3)c(C)c2)C1]

N-(4-(3-(1-acryloylpiperidin-3-yl)pyridin-4-yl)-2-methylbenzyl)-5-(1-methylcyclopropyl)-1,2,4-oxadiazole-3-carboxamide was obtained from tert-butyl 3-(4-(3-methyl-4-((5-(1-methylcyclopropyl)-1,2,4-oxadiazole-3-carboxamido)methyl)phenyl)pyridin-3-yl)piperidine-1-carboxylate, following the procedure described in Example 23, steps 2 and 3. The compound was purified by prep HPLC (Method C, organic gradient: 5-55%) LCMS m/z=486.1 (M+H)+. ¹H NMR (500 MHz, DMSO-d₆) δ: 9.38 (br t, 1H), 8.72 (d, 1H), 8.53-8.42 (m, 1H), 7.36-7.26 (m, 1H), 7.24-7.10 (m, 3H), 6.81-6.41 (m, 1H), 6.11-5.89 (m, 1H), 5.69-5.49 (m, 1H), 4.51-4.35 (m, 3H), 4.03 (br d, 1H), 3.25-3.08 (m, 1H), 2.79-2.57 (m, 2H), 2.41-2.26 (m, 4H), 1.99-1.62 (m, 3H), 1.54 (s, 3H), 1.43-1.33 (m, 2H), 1.22-1.14 (m, 2H).

### Examples 27 and 28: (R)-N-(4-(3-(1-acryloylpiperidin-3-yl)pyridin-4-yl)-2-methylbenzyl)-5-(1-methylcyclopropyl)-1,2,4-oxadiazole-3-carboxamide and (S)—N-(4-(3-(1-acryloylpiperidin-3-yl)pyridin-4-yl)-2-methylbenzyl)-5-(1-methylcyclopropyl)-1,2,4-oxadiazole-3-carboxamide

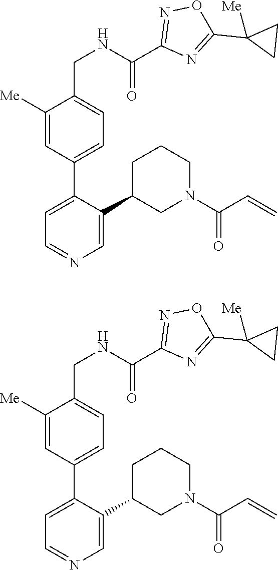 [SMILES: C=CC(=O)N1CCC[C@H](c2cnccc2-c2ccc(CNC(=O)c3noc(C4(C)CC4)n3)c(C)c2)C1 ; C=CC(=O)N1CCC[C@@H](c2cnccc2-c2ccc(CNC(=O)c3noc(C4(C)CC4)n3)c(C)c2)C1]

### Stereochemistry Arbitrarily Assigned

N-(4-(3-(1-acryloylpiperidin-3-yl)pyridin-4-yl)-2-methylbenzyl)-5-(1-methylcyclopropyl)-1,2,4-oxadiazole-3-carboxamide (10 mg, 21 μmol) from Example 26 was separated by SFC (Method D) to provide (R)-N-(4-(3-(1-acryloylpiperidin-3-yl)pyridin-4-yl)-2-methylbenzyl)-5-(1-methylcyclopropyl)-1,2,4-oxadiazole-3-carboxamide and (S)—N-(4-(3-(1-acryloylpiperidin-3-yl)pyridin-4-yl)-2-methylbenzyl)-5-(1-methylcyclopropyl)-1,2,4-oxadiazole-3-carboxamide. The first peak (Example 27) eluted at Rt=3.58 min (2.5 mg) and second peak (Example 28) eluted at Rt=4.18 min (2.3 mg).

### Example 29: N-(4-(3-(1-acryloylpiperidin-3-yl)pyridin-4-yl)-2-methylbenzyl)-3-(1-methylcyclopropyl)-1,2,4-oxadiazole-5-carboxamide

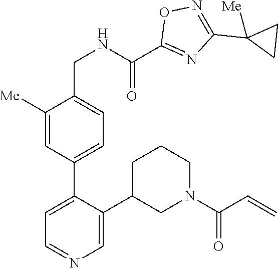 [SMILES: C=CC(=O)N1CCCC(c2cnccc2-c2ccc(CNC(=O)c3nc(C4(C)CC4)no3)c(C)c2)C1]

N-(4-(3-(1-acryloylpiperidin-3-yl)pyridin-4-yl)-2-methylbenzyl)-3-(1-methylcyclopropyl)-1,2,4-oxadiazole-5-carboxamide was obtained from Intermediate 8: tert-butyl 3-(4-(4-(aminomethyl)-3-methylphenyl)pyridin-3-yl)piperidine-1-carboxylate and ethyl 3-(1-methylcyclopropyl)-1,2,4-oxadiazole-5-carboxylate, following the steps described in Example 1, except the compound was purified by prep HPLC (Method C, organic gradient: 5-55%). LCMS m/z=486.1 (M+H)+. ¹H NMR (500 MHz, DMSO-d₆) δ: 9.75 (br d, 1H), 8.72 (d, 1H), 8.53-8.40 (m, 1H), 7.43-7.27 (m, 1H), 7.27-7.11 (m, 3H), 6.81-6.44 (m, 1H), 6.13-5.87 (m, 1H), 5.73-5.44 (m, 1H), 4.56-4.36 (m, 3H), 4.11-3.94 (m, 1H), 3.25-3.06 (m, 1H), 2.81-2.57 (m, 2H), 2.43-2.31 (m, 4H), 2.02-1.64 (m, 3H), 1.48 (s, 3H), 1.22-1.14 (m, 2H), 0.99 (br s, 2H).

### Examples 30 and 31: (R)-N-(4-(3-(1-acryloylpiperidin-3-yl)pyridin-4-yl)-2-methylbenzyl)-3-(1-methylcyclopropyl)-1,2,4-oxadiazole-5-carboxamide and (S)-N-(4-(3-(1-acryloylpiperidin-3-yl)pyridin-4-yl)-2-methylbenzyl)-3-(1-methylcyclopropyl)-1,2,4-oxadiazole-5-carboxamide

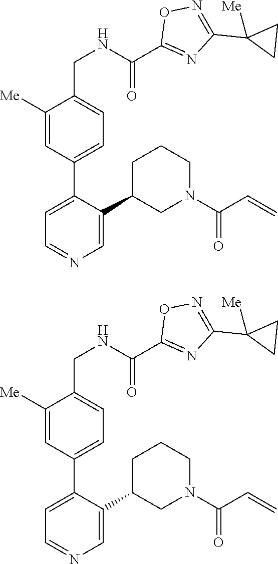 [SMILES: C=CC(=O)N1CCC[C@H](c2cnccc2-c2ccc(CNC(=O)c3nc(C4(C)CC4)no3)c(C)c2)C1 ; C=CC(=O)N1CCC[C@@H](c2cnccc2-c2ccc(CNC(=O)c3nc(C4(C)CC4)no3)c(C)c2)C1]

### Stereochemistry Arbitrarily Assigned

N-(4-(3-(1-acryloylpiperidin-3-yl)pyridin-4-yl)-2-methylbenzyl)-3-(1-methylcyclopropyl)-1,2,4-oxadiazole-5-carboxamide (15 mg, 31 μmol) from Example 29 was separated by SFC (Method D) to provide (R)-N-(4-(3-(1-acryloylpiperidin-3-yl)pyridin-4-yl)-2-methylbenzyl)-3-(1-methylcyclopropyl)-1,2,4-oxadiazole-5-carboxamide and (S)-N-(4-(3-(1-acryloylpiperidin-3-yl)pyridin-4-yl)-2-methylbenzyl)-3-(1-methylcyclopropyl)-1,2,4-oxadiazole-5-carboxamide. The first peak (Example 30) eluted at Rt=1.92 min (7.2 mg) and second peak (Example 31) eluted at Rt=2.14 min (7.7 mg).

### Example 32: N-(4-(3-(1-acryloylpiperidin-3-yl)pyridin-4-yl)-2-methylbenzyl)-5-(tert-butyl)isoxazole-3-carboxamide

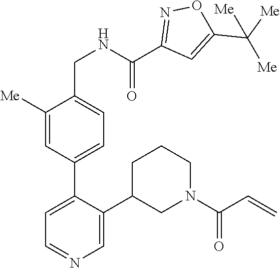 [SMILES: C=CC(=O)N1CCCC(c2cnccc2-c2ccc(CNC(=O)c3cc(C(C)(C)C)on3)c(C)c2)C1]

N-(4-(3-(1-acryloylpiperidin-3-yl)pyridin-4-yl)-2-methylbenzyl)-5-(tert-butyl)isoxazole-3-carboxamide was obtained from Intermediate 8: tert-butyl 3-(4-(4-(aminomethyl)-3-methylphenyl)pyridin-3-yl)piperidine-1-carboxylate and 5-(tert-butyl)isoxazole-3-carboxylic acid, following the steps described in Example 1. LCMS m/z=487.1 (M+H)+. ¹H NMR (500 MHz, DMSO-d₆) δ: 9.21 (br d, 1H), 8.72 (d, 1H), 8.54-8.40 (m, 1H), 7.35-7.25 (m, 1H), 7.25-7.11 (m, 3H), 6.81-6.44 (m, 1H), 6.60 (s, 1H), 6.13-5.88 (m, 1H), 5.71-5.47 (m, 1H), 4.53-4.34 (m, 3H), 4.03 (br d, 1H), 3.61-3.41 (m, 1H), 3.26-3.04 (m, 1H), 2.81-2.58 (m, 1H), 2.41-2.18 (m, 3H), 2.00-1.78 (m, 2H), 1.78-1.66 (m, 1H), 1.33 (s, 9H), 1.28-1.14 (m, 1H).

### Example 33 and 34, (R)-N-(4-(3-(1-acryloylpiperidin-3-yl)pyridin-4-yl)-2-methylbenzyl)-5-(tert-butyl)isoxazole-3-carboxamide and (S)—N-(4-(3-(1-acryloylpiperidin-3-yl)pyridin-4-yl)-2-methylbenzyl)-5-(tert-butyl)isoxazole-3-carboxamide

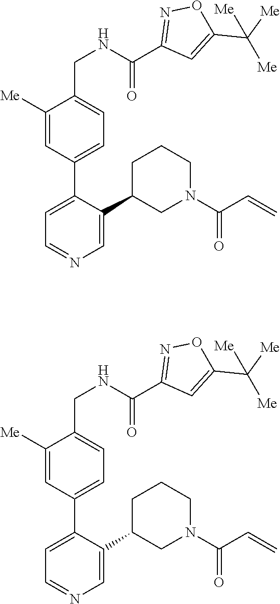 [SMILES: C=CC(=O)N1CCC[C@H](c2cnccc2-c2ccc(CNC(=O)c3cc(C(C)(C)C)on3)c(C)c2)C1 ; C=CC(=O)N1CCC[C@@H](c2cnccc2-c2ccc(CNC(=O)c3cc(C(C)(C)C)on3)c(C)c2)C1]

### Stereochemistry Arbitrarily Assigned

N-(4-(3-(1-acryloylpiperidin-3-yl)pyridin-4-yl)-2-methylbenzyl)-5-(tert-butyl)isoxazole-3-carboxamide (10 mg, 21 μmol) from Example 32 was further purified by SFC (Method D) to provide (R)-N-(4-(3-(1-acryloylpiperidin-3-yl)pyridin-4-yl)-2-methylbenzyl)-5-(tert-butyl)isoxazole-3-carboxamide and (S)—N-(4-(3-(1-acryloylpiperidin-3-yl)pyridin-4-yl)-2-methylbenzyl)-5-(tert-butyl)isoxazole-3-carboxamide. The first peak (Example 33) eluted at Rt=2.35 min (4.8 mg) and second peak (Example 34) eluted at Rt=2.68 min (4.9 mg).

### Example 35: N-(4-(3-(1-acryloylpiperidin-4-yl)pyridin-4-yl)-2-methylbenzyl)-5-(tert-butyl)-1,2,4-oxadiazole-3-carboxamide

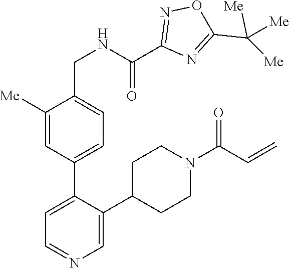 [SMILES: C=CC(=O)N1CCC(c2cnccc2-c2ccc(CNC(=O)c3noc(C(C)(C)C)n3)c(C)c2)CC1]

### 1. Synthesis of tert-butyl 4-(4-(4-((5-(tert-butyl)-1,2,4-oxadiazole-3-carboxamido)methyl)-3-methylphenyl)pyridin-3-yl)piperidine-1-carboxylate

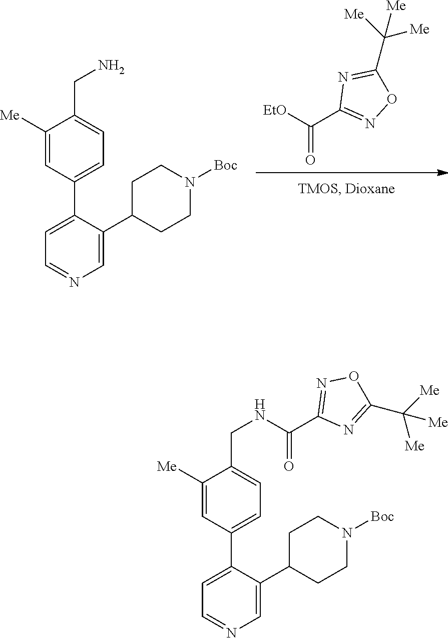 [SMILES: Cc1cc(-c2ccncc2C2CCN(C(=O)OC(C)(C)C)CC2)ccc1CN ; CCOC(=O)c1noc(C(C)(C)C)n1 ; Cc1cc(-c2ccncc2C2CCN(C(=O)OC(C)(C)C)CC2)ccc1CNC(=O)c1noc(C(C)(C)C)n1]

A solution of Intermediate 9: tert-butyl 4-(4-(4-(aminomethyl)-3-methylphenyl)pyridin-3-yl)piperidine-1-carboxylate (100 mg, 0.26 mmol) in dioxane (2 mL) followed by TMOS (120 mg, 0.79 mmol) were added to ethyl 5-(tert-butyl)-1,2,4-oxadiazole-3-carboxylate (100 mg, 0.50 mmol) and the reaction was stirred at 90° C. for 20 h. The cooled reaction was concentrated in vacuo and the residue was purified by silica gel column chromatography, eluting with heptanes to EtOAc to give tert-butyl 4-(4-(4-((5-(tert-butyl)-1,2,4-oxadiazole-3-carboxamido)methyl)-3-methylphenyl)pyridin-3-yl)piperidine-1-carboxylate (8 mg, 6% yield). LCMS m/z=534.2 (M+H)+.

### 2. Synthesis of 5-(tert-butyl)-N-(2-methyl-4-(3-(piperidin-4-yl)pyridin-4-yl)benzyl)-1,2,4-oxadiazole-3-carboxamide hydrochloride

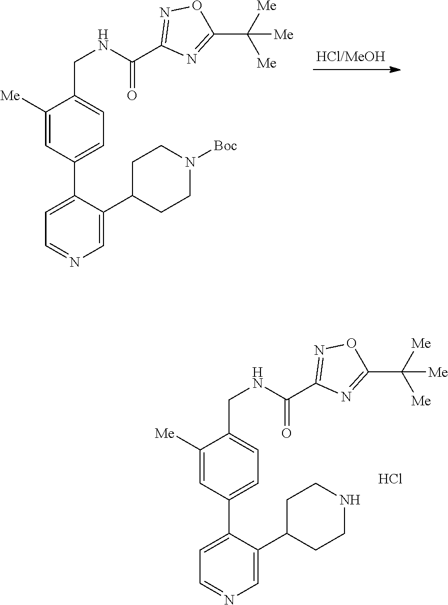 [SMILES: Cc1cc(-c2ccncc2C2CCN(C(=O)OC(C)(C)C)CC2)ccc1CNC(=O)c1noc(C(C)(C)C)n1 ; Cc1cc(-c2ccncc2C2CCNCC2)ccc1CNC(=O)c1noc(C(C)(C)C)n1 ; Cl]

A solution of tert-butyl 4-(4-(4-((5-(tert-butyl)-1,2,4-oxadiazole-3-carboxamido)methyl)-3-methylphenyl)pyridin-3-yl)piperidine-1-carboxylate (8 mg, 15 μmol) in MeOH (2 mL) and HCl (4 M, 500 μL) was left to stand at rt for 18 h. The reaction was diluted with EtOAc (5 mL), evaporated under reduced pressure, and azeotroped with EtOAc to give 5-(tert-butyl)-N-(2-methyl-4-(3-(piperidin-4-yl)pyridin-4-yl)benzyl)-1,2,4-oxadiazole-3-carboxamide hydrochloride (8 mg, crude) as a white solid. LCMS m/z=434.2 (M+H)+.

### 3. Synthesis of N-(4-(3-(1-acryloylpiperidin-4-yl)pyridin-4-yl)-2-methylbenzyl)-5-(tert-butyl)-1,2,4-oxadiazole-3-carboxamide

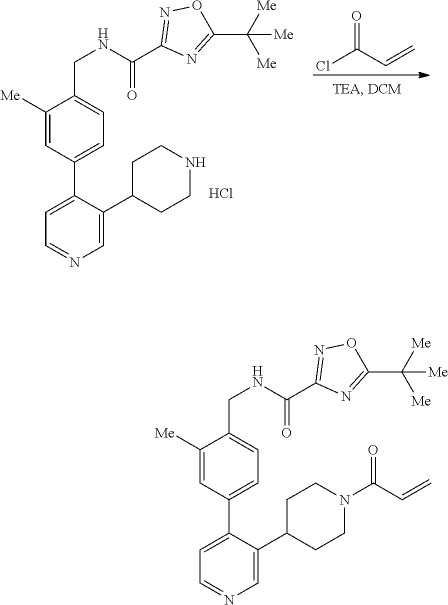

To 5-(tert-butyl)-N-(2-methyl-4-(3-(piperidin-4-yl)pyridin-4-yl)benzyl)-1,2,4-oxadiazole-3-carboxamide hydrochloride (8 mg, 15 μmol) was added DCM (5 mL) and TEA (5 mg, 47 μmol) and the solution was cooled to <0° C. Acryloyl chloride (1.4 mg, 15 μmol) was added, the reaction was stirred for 15 min, and then NaHCO₃ (5 mL) was added and the reaction allowed to warm to rt. The phases were separated, the aqueous layer extracted with DCM (5 mL), and the combined organic layers were evaporated under reduced pressure. The crude material was dissolved in DMSO (2.3 mL), filtered through a 0.2 μm syringe filter, and purified by prep HPLC (Method C, organic gradient: 5-55%) to give N-(4-(3-(1-acryloylpiperidin-4-yl)pyridin-4-yl)-2-methylbenzyl)-5-(tert-butyl)-1,2,4-oxadiazole-3-carboxamide. LCMS m/z=488.2 (M+H)+. ¹H NMR (500 MHz, DMSO-d₆) δ: 9.47 (br t, 1H), 8.65 (s, 1H), 8.45 (br d, 1H), 7.35 (br d, 1H), 7.25-7.12 (m, 3H), 6.89-6.67 (m, 1H), 6.19-6.02 (m, 1H), 5.76-5.57 (m, 1H), 4.66-4.41 (m, 3H), 4.26-4.00 (m, 1H), 3.03-2.81 (m, 2H), 2.47-2.41 (m, 1H), 2.40 (s, 3H), 1.83-1.59 (m, 4H), 1.54-1.31 (m, 9H).

### Example 36: N-(4-(3-(1-acryloylpiperidin-4-yl)pyridin-4-yl)-2-methylbenzyl)-3-(tert-butyl)-1,2,4-oxadiazole-5-carboxamide

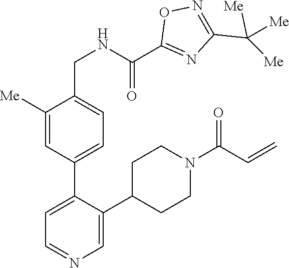 [SMILES: C=CC(=O)N1CCC(c2cnccc2-c2ccc(CNC(=O)c3nc(C(C)(C)C)no3)c(C)c2)CC1]

N-(4-(3-(1-acryloylpiperidin-4-yl)pyridin-4-yl)-2-methylbenzyl)-3-(tert-butyl)-1,2,4-oxadiazole-5-carboxamide was obtained from Intermediate 9: tert-butyl 4-(4-(4-(aminomethyl)-3-methylphenyl)pyridin-3-yl)piperidine-1-carboxylate and ethyl 3-tert-butyl-1,2,4-oxadiazole-5-carboxylate, following the steps described in Example 35. The compound was purified by prep HPLC (Method C, organic gradient: 5-55%) to give the title compound. LCMS m/z=488.1 (M+H)+. ¹H NMR (500 MHz, DMSO-d₆) δ: 9.86 (s, 1H), 8.64 (s, 1H), 8.45 (br d, 1H), 7.39 (br d, 1H), 7.25-7.15 (m, 3H), 6.86-6.70 (m, 1H), 6.12 (s, 1H), 5.76-5.55 (m, 1H), 4.53 (br d, 3H), 4.23-4.03 (m, 1H), 3.02-2.82 (m, 2H), 2.45-2.43 (m, 1H), 2.40 (s, 3H), 1.82-1.56 (m, 4H), 1.47-1.27 (m, 9H).

### Example 37: 2-(tert-butyl)-N-(2-methyl-4-(3-(3-(N-methylacrylamido)piperidin-1-yl)pyridin-4-yl)benzyl)oxazole-4-carboxamide

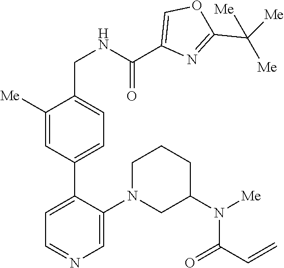 [SMILES: C=CC(=O)N(C)C1CCCN(c2cnccc2-c2ccc(CNC(=O)c3coc(C(C)(C)C)n3)c(C)c2)C1]

### 1. Synthesis of tert-butyl (1-(4-(4-((2-(tert-butyl)oxazole-4-carboxamido)methyl)-3-methylphenyl)pyridin-3-yl)piperidin-3-yl)(methyl)carbamate

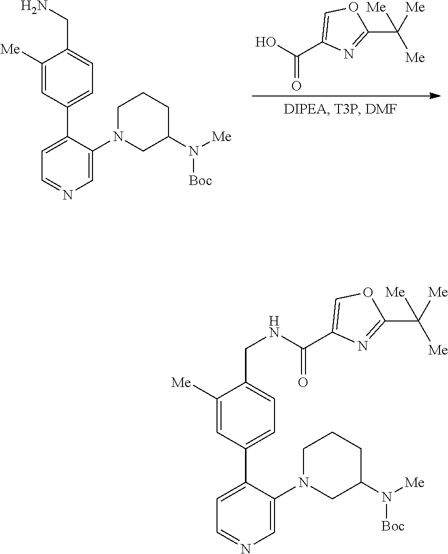 [SMILES: Cc1cc(-c2ccncc2N2CCCC(N(C)C(=O)OC(C)(C)C)C2)ccc1CN ; CC(C)(C)c1nc(C(=O)O)co1 ; Cc1cc(-c2ccncc2N2CCCC(N(C)C(=O)OC(C)(C)C)C2)ccc1CNC(=O)c1coc(C(C)(C)C)n1]

DMF (1 mL), DIPEA (80 mg, 620 μmol) and T3P (376 mg, 590 μmol, 50% purity) were added to 2-(tert-butyl)oxazole-4-carboxylic acid (100 mg, 591 μmol) and Intermediate 10: tert-butyl (1-(4-(4-(aminomethyl)-3-methylphenyl)pyridin-3-yl)piperidin-3-yl)(methyl)carbamate (120 mg, 292 μmol) and the reaction was stirred at rt for 20 h. The reaction was diluted with EtOAc (5 mL) and washed with NaHCO₃ (10 mL) and the layers were separated. The aqueous layer was extracted with EtOAc (2×5 mL) and the combined organic layers were evaporated under reduced pressure. The crude product was purified by silica gel column chromatography eluting with heptanes to EtOAc to give tert-butyl (1-(4-(4-((2-(tert-butyl)oxazole-4-carboxamido)methyl)-3-methylphenyl)pyridin-3-yl)piperidin-3-yl)(methyl)carbamate as a clear oil. (164 mg, 100% yield). LCMS m/z=562.3 (M+H)+.

### 2. Synthesis of 2-(tert-butyl)-N-(2-methyl-4-(3-(3-(methylamino)piperidin-1-yl)pyridin-4-yl)benzyl)oxazole-4-carboxamide hydrochloride

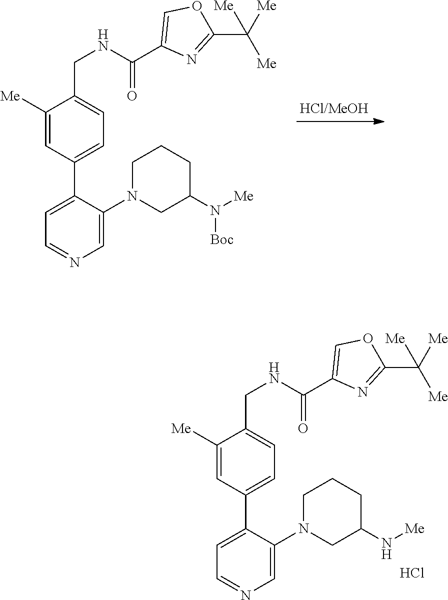 [SMILES: Cc1cc(-c2ccncc2N2CCCC(N(C)C(=O)OC(C)(C)C)C2)ccc1CNC(=O)c1coc(C(C)(C)C)n1 ; CNC1CCCN(c2cnccc2-c2ccc(CNC(=O)c3coc(C(C)(C)C)n3)c(C)c2)C1 ; Cl]

A solution of tert-butyl (1-(4-(4-((2-(tert-butyl)oxazole-4-carboxamido)methyl)-3-methylphenyl)pyridin-3-yl)piperidin-3-yl)(methyl)carbamate (164 mg, 292 μmol) in 1.25 M HCl/MeOH (5.0 mL) was stirred at rt for 20 h. EtOAc (5 mL) was added, the solution concentrated in vacuo and azeotroped with EtOAc (10 mL) to give 2-(tert-butyl)-N-(2-methyl-4-(3-(3-(methylamino)piperidin-1-yl)pyridin-4-yl)benzyl)oxazole-4-carboxamide hydrochloride (164 mg, crude) as a white solid. LCMS m/z=462.2 (M+H)+.

### 3. Synthesis of 2-(tert-butyl)-N-(2-methyl-4-(3-(3-(N-methylacrylamido)piperidin-1-yl)pyridin-4-yl)benzyl)oxazole-4-carboxamide

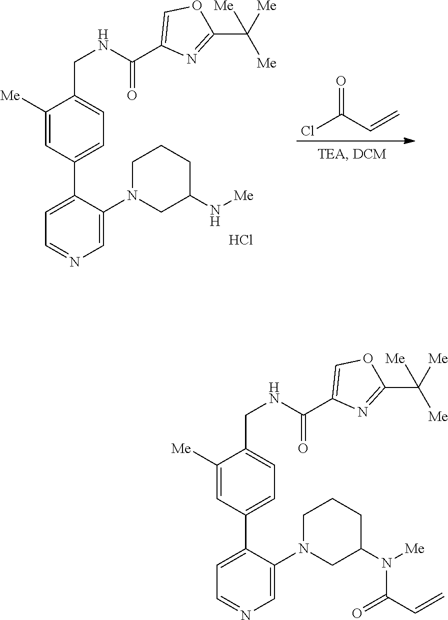

A solution of 2-(tert-butyl)-N-(2-methyl-4-(3-(3-(methylamino)piperidin-1-yl)pyridin-4-yl)benzyl)oxazole-4-carboxamide hydrochloride (164 mg from previous reaction) and TEA (213 μL, 1.53 mmol) in DCM (5 mL) was cooled to −10° C. Acryloyl chloride (25 μL, 307 μmol) was added, the reaction was stirred for 5 mins, then quenched with sat. aq. NaHCO₃ (10 mL), and the biphasic mixture was stirred vigorously while warming to rt. The layers were separated, and the aqueous phase was extracted with EtOAc (3×10 mL). The combined organic layers were concentrated in vacuo and the residue was purified by silica gel column chromatography eluting with EtOAc/EtOH (100/0 to 75/25) to give 2-(tert-butyl)-N-(2-methyl-4-(3-(3-(N-methylacrylamido)piperidin-1-yl)pyridin-4-yl)benzyl)oxazole-4-carboxamide as a colorless glass-like solid (107 mg, 68% yield). LCMS m/z=516.2 (M+H)+. ¹H NMR (500 MHz, DMSO-d₆) δ: 8.54 (br d, 2H), 8.35 (s, 1H), 8.30-8.17 (m, 1H), 7.54 (br s, 2H), 7.37-7.27 (m, 1H), 7.23-7.11 (m, 1H), 6.18-6.04 (m, 1H), 6.00-5.87 (m, 1H), 5.72-5.54 (m, 1H), 4.57-4.34 (m, 2H), 3.68 (br s, 1H), 3.16-3.00 (m, 1H), 2.99-2.83 (m, 3H), 2.77-2.59 (m, 3H), 2.38 (s, 3H), 1.81-1.45 (m, 4H), 1.37 (s, 9H).

### Examples 38 to 41

The compounds in the following table were prepared from Intermediate 10: tert-butyl (1-(4-(4-(aminomethyl)-3-methylphenyl)pyridin-3-yl)piperidin-3-yl)(methyl)carbamate and the appropriate carboxylic acid, following the steps described in Example 37.

| Ex No | Structure, Starting Material, Name | Yield, Data |
|---|---|---|
| 38 | 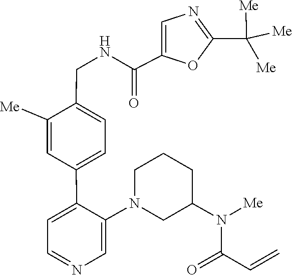 [SMILES: C=CC(=O)N(C)C1CCCN(c2cnccc2-c2ccc(CNC(=O)c3cnc(C(C)(C)C)o3)c(C)c2)C1] SM: 2-(tert-butyl)oxazole-5-carboxylic acid and 2-(tert-butyl)-N-(2-methyl-4- (3-(3-(N-methylacrylamido)piperidin- 1-yl)pyridin-4-yl)benzyl)oxazole-5- carboxamide | 82 mg, 64% yield LCMS m/z = 516.2 (M + H)+ 1H NMR (500 MHZ, DMSO-d₆) δ: 8.96 (s, 1H), 8.35 (s, 1H), 8.30-8.18 (m, 1H), 7.80-7.65 (m, 1H), 7.63-7.45 (m, 2H), 7.42-7.28 (m, 1H), 7.22-7.08 (m, 1H), 6.21-6.01 (m, 1H), 6.00-5.85 (m, 1H), 5.73-5.49 (m, 1H), 4.57-4.34 (m, 2H), 3.68 (br s, 1H), 3.15-2.97 (m, 2H), 2.91 (br s, 2H), 2.76-2.62 (m, 3H), 2.38 (s, 4H), 1.80-1.44 (m, 3H), 1.36 (s, 9H). |
| 39 | 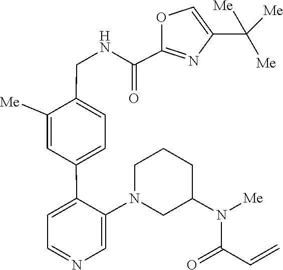 [SMILES: C=CC(=O)N(C)C1CCCN(c2cnccc2-c2ccc(CNC(=O)c3nc(C(C)(C)C)co3)c(C)c2)C1] SM: potassium 4-(tert-butyl)oxazole-2- carboxylate and 4-(tert-butyl)-N-(2- methyl-4-(3-(3-(N-methylacrylamido)- piperidin-1-yl)pyridin-4-yl)benzyl)- oxazole-2-carboxamide | 47 mg, 58% yield LCMS m/z = 516.2 (M + H)+ 1H NMR (500 MHZ, DMSO-d₆) δ: 9.27 (br s, 1H), 8.35 (s, 1H), 8.26 (br s, 1H), 8.03 (br s, 1H), 7.54 (br s, 2H), 7.34 (br s, 1H), 7.17 (br s, 1H), 6.09 (br s, 1H), 5.93 (br d, 1H), 5.68-5.53 (m, 1H), 4.46 (br d, 2H), 3.68 (br s, 1H), 3.14-2.96 (m, 2H), 2.91 (br s, 2H), 2.70 (br s, 3H), 2.38 (s, 3H), 1.69 (br s, 1H), 1.60 (br s, 3H), 1.26 (s, 9H). |
| 40 | 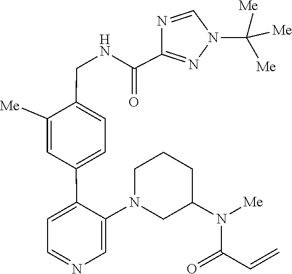 [SMILES: C=CC(=O)N(C)C1CCCN(c2cnccc2-c2ccc(CNC(=O)c3ncn(C(C)(C)C)n3)c(C)c2)C1] SM: 1-(tert-butyl)-1H-1,2,4-triazole-3- carboxylic acid and 1-(tert-butyl)-N-(2- methyl-4-(3-(3-(N-methylacrylamido)- piperidin-1-yl)pyridin-4-yl)benzyl)-1H- 1,2,4-triazole-3-carboxamide | 97 mg, 71% yield LCMS m/z = 516.2 (M + H)+ No NMR data reported. |
| 41 | 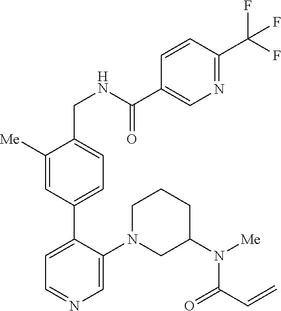 [SMILES: C=CC(=O)N(C)C1CCCN(c2cnccc2-c2ccc(CNC(=O)c3ccc(C(F)(F)F)nc3)c(C)c2)C1] SM: 6-(trifluoromethyl)pyridine-3- carboxylic acid and N-(2-methyl-4-(3- (3-(N-methylacrylamido)piperidin-1- yl)pyridin-4-yl)benzyl)-6- (trifluoromethyl)nicotinamide | 86.0 mg, 53% yield LCMS m/z = 538.2 (M + H)+ No NMR data reported. |

### Example 42: 1-(tert-butyl)-N-(2-methyl-4-(3-(3-(N-methylacrylamido)piperidin-1-yl)pyridin-4-yl)benzyl)-1H-pyrazole-4-carboxamide

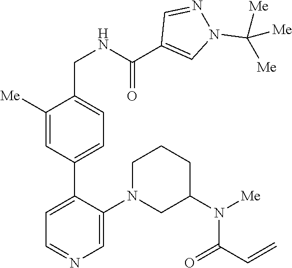 [SMILES: C=CC(=O)N(C)C1CCCN(c2cnccc2-c2ccc(CNC(=O)c3cnn(C(C)(C)C)c3)c(C)c2)C1]

### 1. Synthesis of tert-butyl (]-(4-(4-((]-(tert-butyl)-] H-pyrazole-4-carboxamido)methyl)-3-methylphenyl)pyridin-3-yl)piperidin-3-yl)(methyl)carbamate

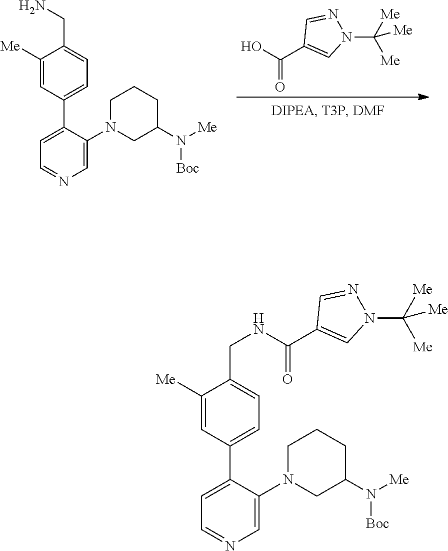 [SMILES: Cc1cc(-c2ccncc2N2CCCC(N(C)C(=O)OC(C)(C)C)C2)ccc1CN ; CC(C)(C)n1cc(C(=O)O)cn1 ; Cc1cc(-c2ccncc2N2CCCC(N(C)C(=O)OC(C)(C)C)C2)ccc1CNC(=O)c1cnn(C(C)(C)C)c1]

DIPEA (0.4 mL, 2.29 mmol) was added dropwise to a solution of 1-(tert-butyl)-1H-pyrazole-4-carboxylic acid (161 mg, 960 μmol) and Intermediate 10: tert-butyl (1-(4-(4-(aminomethyl)-3-methylphenyl)pyridin-3-yl)piperidin-3-yl)(methyl)carbamate (130 mg, 318 μmol) in anhydrous DMF (0.5 mL). T3P (50% solution in DMF, 1.1 mL, 1.85 mmol) was then added and the reaction was stirred at rt for 22 h. The mixture was diluted with EtOAc, washed with aq. sat. NaHCO₃, and brine, and the aqueous layers were re-extracted with EtOAc (2 x). The combined organic extracts were dried over MgSO₄, filtered and concentrated in vacuo. The residue was purified by silica gel column chromatography eluting with (3:1 EtOAc:EtOH) in heptanes (5/95 to 30/70) to give tert-butyl (1-(4-(4-((1-(tert-butyl)-1H-pyrazole-4-carboxamido)methyl)-3-methylphenyl)pyridin-3-yl)piperidin-3-yl)(methyl)carbamate as a yellow oil (27 mg, 15% yield). LCMS m/z=561.3 (M+H)+.

### 2. Synthesis of 1-(tert-butyl)-N-(2-methyl-4-(3-(3-(methylamino)piperidin-1-yl)pyridin-4-yl)benzyl)-1H-pyrazole-4-carboxamide hydrochloride

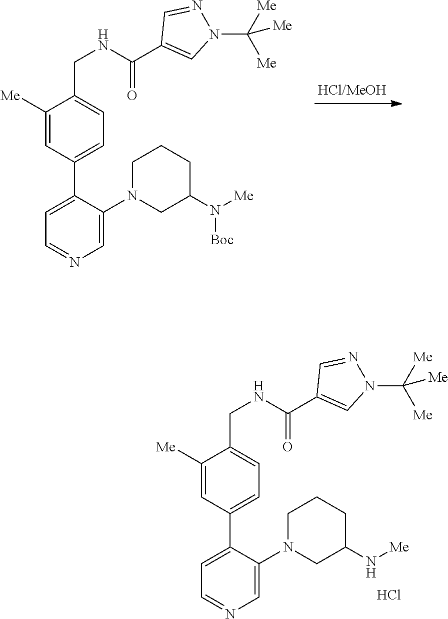 [SMILES: Cc1cc(-c2ccncc2N2CCCC(N(C)C(=O)OC(C)(C)C)C2)ccc1CNC(=O)c1cnn(C(C)(C)C)c1 ; CNC1CCCN(c2cnccc2-c2ccc(CNC(=O)c3cnn(C(C)(C)C)c3)c(C)c2)C1 ; Cl]

A solution of tert-butyl (1-(4-(4-((1-(tert-butyl)-1H-pyrazole-4-carboxamido)methyl)-3-methylphenyl)pyridin-3-yl)piperidin-3-yl)(methyl)carbamate (27 mg, 48 μmol) in MeOH (0.5 mL) and 1.25 M HCl/MeOH (0.5 mL) was stirred at rt for 19 h. The reaction was evaporated under reduced pressure to give 1-(tert-butyl)-N-(2-methyl-4-(3-(3-(methylamino)piperidin-1-yl)pyridin-4-yl)benzyl)-1H-pyrazole-4-carboxamide hydrochloride as a pale yellow film (24 mg, crude), which was carried forward without further purification. LCMS m/z=461.2 (M+H)+.

### 3. Synthesis of 1-(tert-butyl)-N-(2-methyl-4-(3-(3-(N-methylacrylamido)piperidin-1-yl)pyridin-4-yl)benzyl)-1H-pyrazole-4-carboxamide

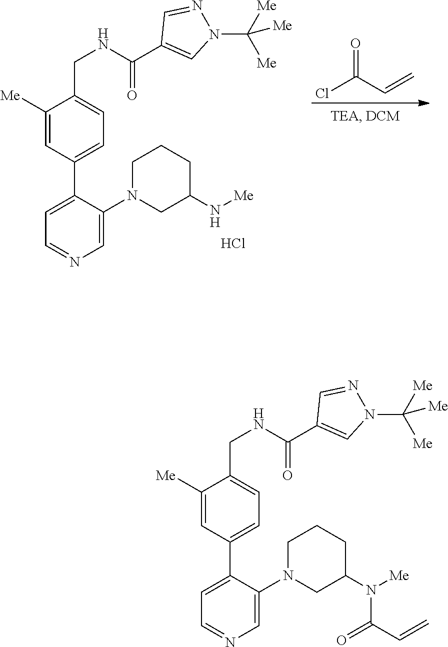

A solution of 1-(tert-butyl)-N-(2-methyl-4-(3-(3-(methylamino)piperidin-1-yl)pyridin-4-yl)benzyl)-1H-pyrazole-4-carboxamide hydrochloride (24 mg, 48 μmol) in DCM (0.5 mL) was cooled to −25° C. and TEA (0.1 mL, 721 μmol) was added dropwise. After 5 mins, acryloyl chloride (0.03 mL, 368 μmol) was added dropwise and the reaction was stirred for 10 mins. The reaction was quenched with sat. aq. NaHCO₃ and the mixture was extracted with DCM (3 x). The combined organic layers were dried over Na₂SO₄, filtered, and the filtrate was concentrated in vacuo. The crude was purified by prep HPLC (Method A2, organic gradient 10-60%) to give 1-(tert-butyl)-N-(2-methyl-4-(3-(3-(N-methylacrylamido)piperidin-1-yl)pyridin-4-yl)benzyl)-1H-pyrazole-4-carboxamide as an off-white solid (3.3 mg, 13% yield). LCMS m/z=515.2 (M+H)+. ¹H NMR (500 MHz, CD₂Cl₂) δ: 8.42-8.21 (m, 2H), 8.16-7.89 (m, 2H), 7.57-7.05 (m, 4H), 6.54-5.86 (m, 2H), 5.50 (br d, 1H), 4.96-4.46 (m, 1H), 4.27 (br s, 1H), 3.72-3.24 (m, 1H), 2.83-2.59 (m, 5H), 2.39 (s, 3H), 2.30-1.84 (m, 2H), 1.79-1.50 (m, 14H).

### Example 43: 1-(tert-butyl)-N-(2-methyl-4-(3-(3-(N-methylacrylamido)piperidin-1-yl)pyridin-4-yl)benzyl)-1H-imidazole-4-carboxamide

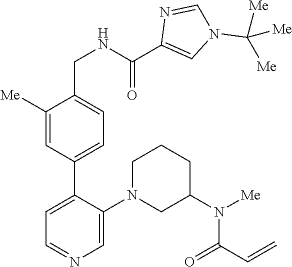 [SMILES: C=CC(=O)N(C)C1CCCN(c2cnccc2-c2ccc(CNC(=O)c3cn(C(C)(C)C)cn3)c(C)c2)C1]

The compound was obtained from 1-(tert-butyl)-1H-imidazole-4-carboxylic acid hydrochloride and Intermediate 10: tert-butyl (1-(4-(4-(aminomethyl)-3-methylphenyl)pyridin-3-yl)piperidin-3-yl)(methyl)carbamate following the steps described in Example 42. The crude product was purified by silica gel column chromatography eluting with 3:1 EtOAc/EtOH in heptanes (15/85 to 35/65) to give 1-(tert-butyl)-N-(2-methyl-4-(3-(3-(N-methylacrylamido)piperidin-1-yl)pyridin-4-yl)benzyl)-1H-imidazole-4-carboxamide as a white solid (27 mg, 59% yield). LCMS m/z=515.2 (M+H)+¹H NMR (500 MHz, DMSO-d₆) δ: 8.39-8.21 (m, 3H), 7.87 (s, 1H), 7.56-7.43 (m, 2H), 7.31 (br d, 1H), 7.16 (br d, 1H), 6.71 (br dd, 1H), 6.09 (br dd, 1H), 5.97-5.58 (m, 3H), 4.56-4.31 (m, 3H), 3.68 (br s, 1H), 3.13-2.87 (m, 4H), 2.79-2.63 (m, 3H), 2.37 (s, 3H), 1.75-1.49 (m, 11H).

### Example 44: 1-(tert-butyl)-N-(2-methyl-4-(3-(3-(N-methylacrylamido)piperidin-1-yl)pyridin-4-yl)benzyl)-1H-pyrazole-3-carboxamide

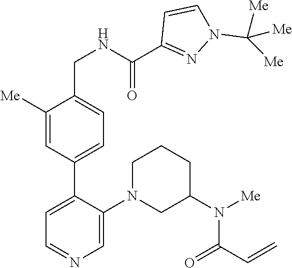 [SMILES: C=CC(=O)N(C)C1CCCN(c2cnccc2-c2ccc(CNC(=O)c3ccn(C(C)(C)C)n3)c(C)c2)C1]

### 1. Synthesis of tert-butyl (1-(4-(4-((1-(tert-butyl)-1H-pyrazole-3-carboxamido)methyl)-3-methylphenyl)pyridin-3-yl)piperidin-3-yl)(methyl)carbamate

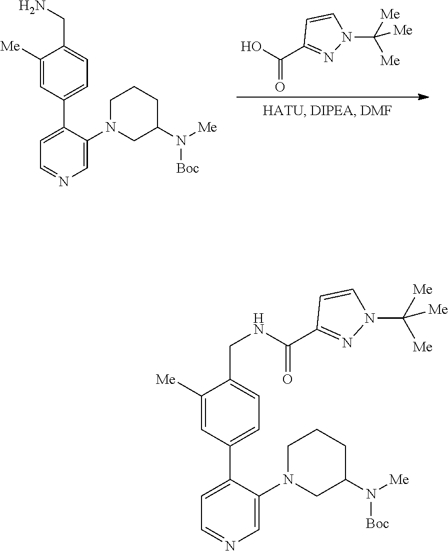 [SMILES: Cc1cc(-c2ccncc2N2CCCC(N(C)C(=O)OC(C)(C)C)C2)ccc1CN ; CC(C)(C)n1ccc(C(=O)O)n1 ; Cc1cc(-c2ccncc2N2CCCC(N(C)C(=O)OC(C)(C)C)C2)ccc1CNC(=O)c1ccn(C(C)(C)C)n1]

DIPEA (0.48 mL, 2.75 mmol), followed by HATU (325 mg, 853 μmol) were added carefully to a solution of 1-(tert-butyl)-pyrazole-3-carboxylic acid (136 mg, 808 μmol) and Intermediate 10: tert-butyl (1-(4-(4-(aminomethyl)-3-methylphenyl)pyridin-3-yl)piperidin-3-yl)(methyl)carbamate (226 mg, 551 μmol) in anhydrous DMF (1 mL) at 0° C. Once addition was complete, the reaction was allowed to warm to rt and was stirred for 2.5 h. The reaction was diluted with EtOAc, was washed sequentially with aq. sat. NaHCO₃ and brine, and then the aqueous layers were re-extracted with EtOAc (2 x). The combined organic extracts were dried over MgSO₄, filtered, and concentrated in vacuo. The residue was purified by silica gel column chromatography eluting with (3:1 EtOAc: EtOH) in heptanes (5/95 to 35/65) to give tert-butyl (1-(4-(4-((1-(tert-butyl)-1H-pyrazole-3-carboxamido)methyl)-3-methylphenyl)pyridin-3-yl)piperidin-3-yl)(methyl)carbamate as a pale yellow solid (279 mg, 90% yield). LCMS m/z=561.3 (M+H)+.

### 2. Synthesis of 1-(tert-butyl)-N-(2-methyl-4-(3-(3-(N-methylacrylamido)piperidin-1-yl)pyridin-4-yl)benzyl)-1H-pyrazole-3-carboxamide

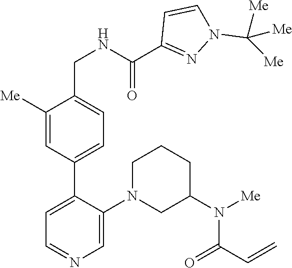 [SMILES: C=CC(=O)N(C)C1CCCN(c2cnccc2-c2ccc(CNC(=O)c3ccn(C(C)(C)C)n3)c(C)c2)C1]

1-(tert-Butyl)-N-(2-methyl-4-(3-(3-(N-methylacrylamido)piperidin-1-yl)pyridin-4-yl)benzyl)-1H-pyrazole-3-carboxamide was obtained as a white solid, from tert-butyl (1-(4-(4-((1-(tert-butyl)-1H-pyrazole-3-carboxamido)methyl)-3-methylphenyl)pyridin-3-yl)piperidin-3-yl)(methyl)carbamate, following the procedure described in Example 42, steps 2 and 3. LCMS m/z=515.2 (M+H)+. ¹H NMR (500 MHz, DMSO-d₆) δ: 8.47-8.27 (m, 3H), 7.92 (d, 1H), 7.59-7.45 (m, 2H), 7.38-7.18 (m, 3H), 6.67 (d, 2H), 6.22-6.04 (m, 1H), 6.02-5.91 (m, 1H), 5.69-5.59 (m, 1H), 4.61-4.28 (m, 2H), 3.15-2.84 (m, 4H), 2.77-2.62 (m, 3H), 2.39 (s, 3H), 1.69 (br d, 1H), 1.65-1.54 (m, 11H).

### Example 45: 5-(tert-butyl)-N-(2-methyl-4-(3-(3-(N-methylacrylamido)piperidin-1-yl)pyridin-4-yl)benzyl)oxazole-2-carboxamide

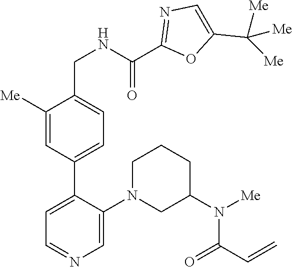 [SMILES: C=CC(=O)N(C)C1CCCN(c2cnccc2-c2ccc(CNC(=O)c3ncc(C(C)(C)C)o3)c(C)c2)C1]

The compound was obtained from potassium 5-(tert-butyl)oxazole-2-carboxylate and Intermediate 10: tert-butyl (1-(4-(4-(aminomethyl)-3-methylphenyl)pyridin-3-yl)piperidin-3-yl)(methyl)carbamate following the steps described in Example 44. The crude product was purified by silica gel column chromatography eluting with 3:1 EtOAc/EtOH in heptane (10/90 to 60/40) to give 5-(tert-butyl)-N-(2-methyl-4-(3-(3-(N-methylacrylamido)piperidin-1-yl)pyridin-4-yl)benzyl)oxazole-2-carboxamide as a white solid (29 mg, 32% yield). LCMS m/z=516.2 (M+H)+. ¹H NMR (500 MHz, DMSO-d₆) δ: 9.37-9.26 (m, 1H), 8.39-8.21 (m, 2H), 7.57-7.30 (m, 3H), 7.23-7.03 (m, 2H), 6.70 (br dd, 1H), 6.16-5.90 (m, 2H), 5.68-5.55 (m, 1H), 4.53-4.37 (m, 2H), 3.68 (br s, 1H), 3.13-2.90 (m, 3H), 2.82-2.62 (m, 3H), 2.38 (s, 3H), 1.89-1.80 (m, 13H).

### Example 46: 3-(tert-butyl)-N-(2-methyl-4-(3-(3-(N-methylacrylamido)piperidin-1-yl)pyridin-4-yl)benzyl)isoxazole-5-carboxamide

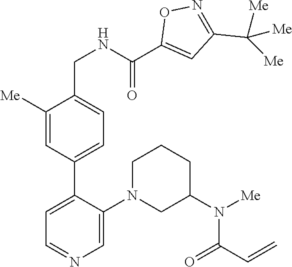 [SMILES: C=CC(=O)N(C)C1CCCN(c2cnccc2-c2ccc(CNC(=O)c3cc(C(C)(C)C)no3)c(C)c2)C1]

The compound was obtained from 3-(tert-butyl)oxazole-5-carboxylic acid and Intermediate 10: tert-butyl (1-(4-(4-(aminomethyl)-3-methylphenyl)pyridin-3-yl)piperidin-3-yl)(methyl)carbamate following the steps described in Example 44. The crude product was purified by silica gel column chromatography eluting with 3:1 EtOAc/EtOH in heptanes (5/95 to 30/70) to give 3-(tert-butyl)-N-(2-methyl-4-(3-(3-(N-methylacrylamido)piperidin-1-yl)pyridin-4-yl)benzyl)isoxazole-5-carboxamide as a white solid (91.2 mg, 57% yield). LCMS m/z=516.2 (M+H)+. ¹H NMR (500 MHz, DMSO-d₆) δ: 9.39-9.28 (m, 1H), 8.38-8.22 (m, 2H), 7.61-7.45 (m, 2H), 7.34 (br d, 1H), 7.17 (br s, 2H), 6.78-5.90 (m, 2H), 5.70-5.49 (m, 1H), 4.55-4.37 (m, 2H), 3.74-3.41 (m, 1H), 3.13-2.96 (m, 1H), 2.91 (br s, 2H), 2.77-2.60 (m, 3H), 2.49-2.34 (m, 4H), 1.80-1.42 (m, 4H), 1.30 (s, 9H).

### Example 47: 1-(tert-butyl)-N-(4-(3-cyano-5-((R)-3-(N-methylacrylamido)piperidin-1-yl)pyridin-4-yl)-3-fluoro-2-methylbenzyl)-1H-1,2,3-triazole-4-carboxamide

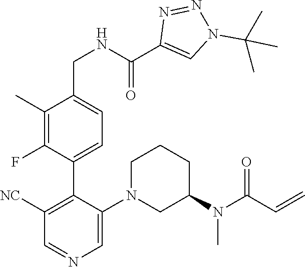 [SMILES: C=CC(=O)N(C)[C@@H]1CCCN(c2cncc(C#N)c2-c2ccc(CNC(=O)c3cn(C(C)(C)C)nn3)c(C)c2F)C1]

### 1. Synthesis of tert-butyl (R)-(1-(4-chloro-5-cyanopyridin-3-yl)piperidin-3-yl)(methyl)carbamate

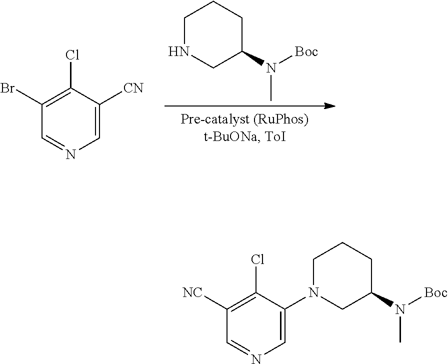 [SMILES: N#Cc1cncc(Br)c1Cl ; CN(C(=O)OC(C)(C)C)[C@@H]1CCCNC1 ; CN(C(=O)OC(C)(C)C)[C@@H]1CCCN(c2cncc(C#N)c2Cl)C1]

To a solution of 5-bromo-4-chloronicotinonitrile (200.00 mg, 919.75 μmol, 1.0 eq.) in toluene (40.00 mL) was added tert-butyl (R)-methyl(piperidin-3-yl)carbamate (197.10 mg, 919.75 μmol, 1.0 eq.) and t-BuONa (176.78 mg, 1.84 mmol, 2.0 eq.) at 15° C. Then Pre-catalyst (RuPhos) (153.85 mg, 183.95 μmol, 0.2 eq.) was added at 15° C. The mixture was stirred at 75° C. for 6 hours under N₂. LCMS showed that the starting material was consumed and the desired product was detected. The mixture was concentrated under vacuum to give crude, which was purified by chromatography column on silica gel (PE/EA=1/0 to 1/1) to give titled compound (170.00 mg, 314.96 μmol, 34.24% yield) as yellow oil. LCMS: m/z=351.3 (M+H⁺).

### 2. Synthesis of tert-butyl ((3R)-1-(4-(4-((1-(tert-butyl)-1H-1,2,3-triazole-4-carboxamido)methyl)-2-fluoro-3-methylphenyl)-5-cyanopyridin-3-yl)piperidin-3-yl)(methyl)carbamate

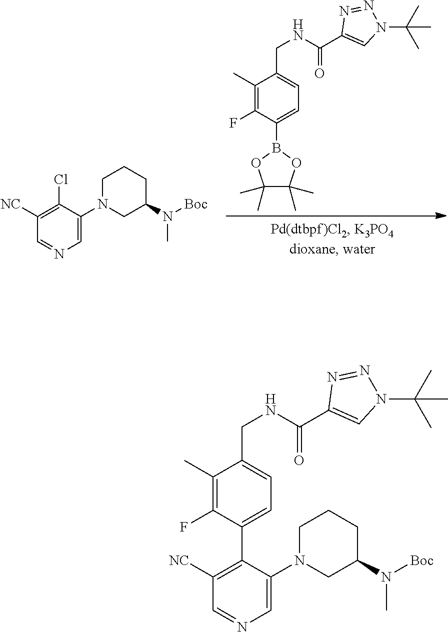 [SMILES: CN(C(=O)OC(C)(C)C)[C@@H]1CCCN(c2cncc(C#N)c2Cl)C1 ; Cc1c(CNC(=O)c2cn(C(C)(C)C)nn2)ccc(B2OC(C)(C)C(C)(C)O2)c1F ; Cc1c(CNC(=O)c2cn(C(C)(C)C)nn2)ccc(-c2c(C#N)cncc2N2CCC[C@@H](N(C)C(=O)OC(C)(C)C)C2)c1F]

To a solution of 1-(tert-butyl)-N-(3-fluoro-2-methyl-4-(4,4,5,5-tetramethyl-1,3,2-dioxaborolan-2-yl)benzyl)-1H-1,2,3-triazole-4-carboxamide (169.68 mg, 407.59 μmol, 1.1 eq.) in a mixture of dioxane (4.00 mL) and water (0.6 mL) was added K₃PO₄ (235.96 mg, 1.11 mmol, 3.0 eq.) and tert-butyl (R)-(1-(4-chloro-5-cyanopyridin-3-yl)piperidin-3-yl)(methyl)carbamate (130.00 mg, 370.54 μmol, 1.0 eq.) at 20° C. Then Pd(dtbpf)Cl₂ (48.30 mg, 74.11 μmol, 0.2 eq.) was added to the mixture at 20° C. The reaction was stirred at 20° C. under N₂ for 2 hours. LCMS showed that the starting material was consumed and the desired product was detected. The mixture was concentrated to give a crude, which was purified by Prep-TLC to give the titled compound (160.00 mg, 42.84% yield) as yellow oil. LCMS: m/z=605.3 (M+H⁺).

### 3. Synthesis of 1-(tert-butyl)-N-(4-(3-cyano-5-((R)-3-(methylamino)piperidin-1-yl)pyridin-4-yl)-3-fluoro-2-methylbenzyl)-1H-1,2,3-triazole-4-carboxamide

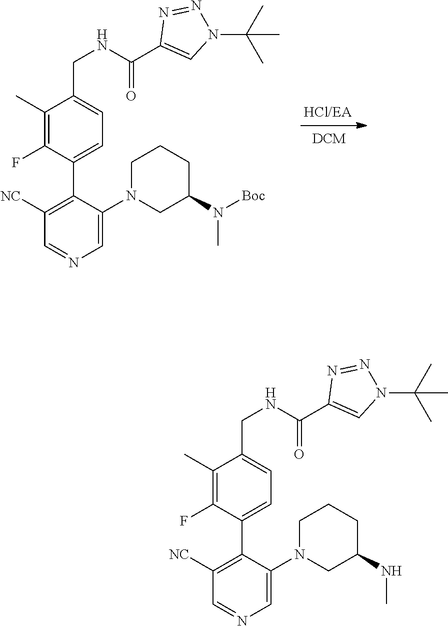

To a solution of tert-butyl ((3R)-1-(4-(4-((1-(tert-butyl)-1H-1,2,3-triazole-4-carboxamido)methyl)-2-fluoro-3-methylphenyl)-5-cyanopyridin-3-yl)piperidin-3-yl)(methyl)carbamate (160.00 mg, 264.59 μmol, 1.0 eq.) in DCM (10.00 mL) was added HCl/EA (1 mL, 4 M) at 20° C. slowly. Then the mixture was stirred at 20° C. for 30 mins. LCMS showed that the starting material was consumed and the desired product was detected. The mixture was concentrated under vacuum to give titled compound (130 mg, crude) as a yellow solid, which was used for the next step directly without further purification. LCMS: m/z=505.2 (M+H⁺).

### 4. Synthesis of 1-(tert-butyl)-N-(4-(3-cyano-5-((R)-3-(N-methylacrylamido)piperidin-1-yl)pyridin-4-yl)-3-fluoro-2-methylbenzyl)-1H-1,2,3-triazole-4-carboxamide

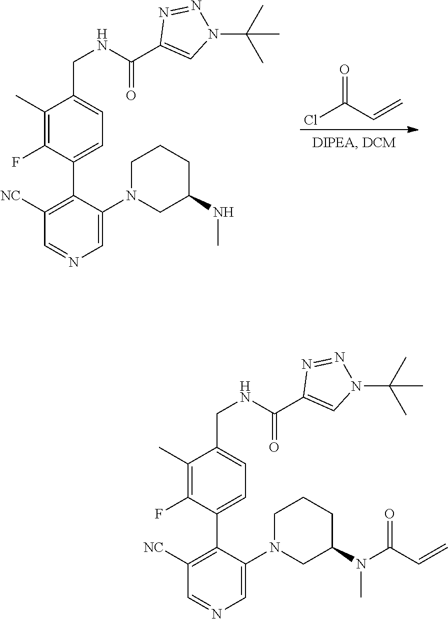

To a solution of 1-(tert-butyl)-N-(4-(3-cyano-5-((R)-3-(methylamino)piperidin-1-yl)pyridin-4-yl)-3-fluoro-2-methylbenzyl)-1H-1,2,3-triazole-4-carboxamide (110 mg, 203.30 μmol, 1.0 eq.) in DCM (20.00 mL) was added DIPEA (52.55 mg, 406.61 μmol, 2.0 eq.) at 20° C. Then acryloyl chloride (20.24 mg, 223.63 μmol, 1.1 eq.) was added to the mixture at 20° C. slowly. Then the mixture was stirred at 20° C. for 30 mins. LCMS showed that the starting material was consumed and the desired product was detected. The mixture was poured into water (50 mL) and extracted with DCM (50 mL×3). The combined organic layers were washed with brine (100 mL), dried over Na₂SO₄, filtered and concentrated under vacuum to give the crude, which was purified by Prep-HPLC (Column: Welch Xtimate C18 150×25 mm×5 m; Condition: water (10 mM NH4HCO3)-ACN, Begin B 35, End B 65; Gradient Time (min): 10; 100% B Hold Time (min): 2; Flow Rate (mL/min): 25) to give the titled compound (48.8 mg, 42.97% yield, 100.00% purity, ee: 100.00%) as a yellow solid. LCMS: m/z=559.4 (M+H⁺). ¹H NMR (500 MHz, DMSO-d6) δ ppm=9.15-9.03 (m, 1H), 8.81-8.56 (m, 3H), 7.44-7.20 (m, 2H), 6.70-5.93 (m, 2H), 5.77-5.59 (m, 1H), 4.65-4.47 (m, 2H), 4.17-3.44 (m, 1H), 2.95-2.53 (m, 7H), 2.30 (d, J=17.5 Hz, 3H), 1.65-1.51 (m, 12H), 1.29-1.12 (m, 1H).

### Example 48: (R)-1-(tert-butyl)-N-(4-(3-cyano-5-(3-(N-methylacrylamido)piperidin-1-yl)pyridin-4-yl)-2-methylbenzyl)-1H-1,2,3-triazole-4-carboxamide

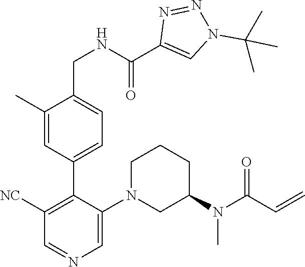 [SMILES: C=CC(=O)N(C)[C@@H]1CCCN(c2cncc(C#N)c2-c2ccc(CNC(=O)c3cn(C(C)(C)C)nn3)c(C)c2)C1]

### 1. Synthesis of tert-butyl (R)-(1-(4-(4-((1-(tert-butyl)-1H-1,2,3-triazole-4-carboxamido)methyl)-3-methylphenyl)-5-cyanopyridin-3-yl)piperidin-3-yl)(methyl)carbamate

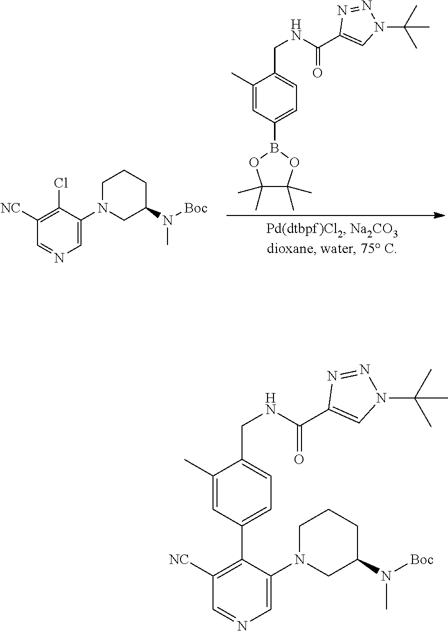 [SMILES: CN(C(=O)OC(C)(C)C)[C@@H]1CCCN(c2cncc(C#N)c2Cl)C1 ; Cc1cc(B2OC(C)(C)C(C)(C)O2)ccc1CNC(=O)c1cn(C(C)(C)C)nn1 ; Cc1cc(-c2c(C#N)cncc2N2CCC[C@@H](N(C)C(=O)OC(C)(C)C)C2)ccc1CNC(=O)c1cn(C(C)(C)C)nn1]

To a solution of 1-(tert-butyl)-N-(2-methyl-4-(4,4,5,5-tetramethyl-1,3,2-dioxaborolan-2-yl)benzyl)-1H-1,2,3-triazole-4-carboxamide (136.24 mg, 342.04 μmol, 1.0 eq.) in dioxane (10.00 mL) and water (2.00 mL) was added tert-butyl (R)-(1-(4-chloro-5-cyanopyridin-3-yl)piperidin-3-yl)(methyl)carbamate (120.00 mg, 342.04 μmol, 1.0 eq.) and K₃PO₄ (217.81 mg, 1.03 mmol, 3.0 eq.) at 15° C. Then Pd(dtbpf)Cl₂ (44.58 mg, 68.41 μmol, 0.2 eq.) was added at 15° C. The mixture was stirred at 15° C. for 3 hours under N₂. LCMS showed that the starting material was consumed and the desired product was detected. The mixture was filtered and concentrated under vacuum to give the crude, which was purified by TLC (PE/EA=1/2) to give titled compound (120.00 mg, 32.89% yield) as yellow oil. LCMS: m/z=587.5 (M+H⁺).

### 2. Synthesis of(R)-1-(tert-butyl)-N-(4-(3-cyano-5-(3-(methylamino)piperidin-1-yl)pyridin-4-yl)-2-methylbenzyl)-1H-1,2,3-triazole-4-carboxamide

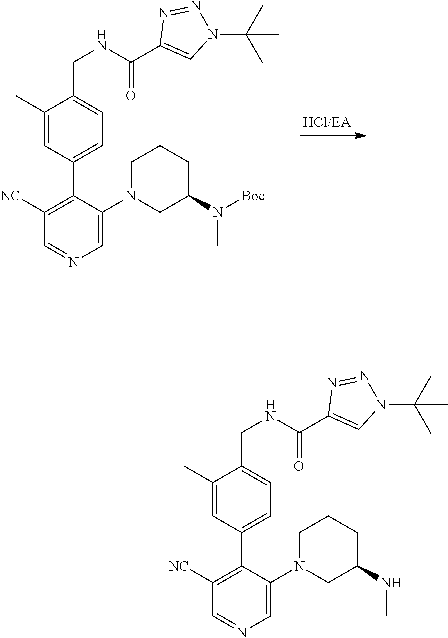

A solution of tert-butyl (R)-(1-(4-(4-((1-(tert-butyl)-1H-1,2,3-triazole-4-carboxamido)methyl)-3-methylphenyl)-5-cyanopyridin-3-yl)piperidin-3-yl)(methyl)carbamate (0.12 g, 204.52 μmol, 1.0 eq.) in DCM (20.00 mL) and HCl/EA (10 mL, 4 M) was stirred at 20° C. for 1 hour. LCMS showed that the starting material was consumed and the desired product was detected. The mixture was concentrated under vacuum to give the titled compound (0.08 g, crude, hydrochloride) as a yellow solid, which was used to next step directly without further purification. LCMS: m/z=487.4 (M+H⁺).

### 3. Synthesis of (R)-1-(tert-butyl)-N-(4-(3-cyano-5-(3-(N-methylacrylamido)piperidin-1-yl)pyridin-4-yl)-2-methylbenzyl)-1H-1, 2,3-triazole-4-carboxamide

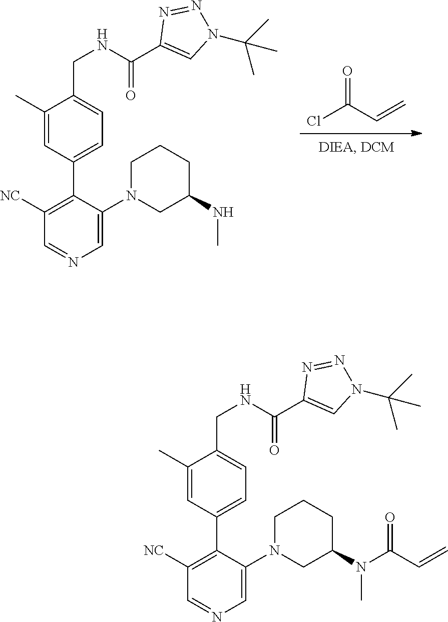

To a solution of (R)-1-(tert-butyl)-N-(4-(3-cyano-5-(3-(methylamino)piperidin-1-yl)pyridin-4-yl)-2-methylbenzyl)-1H-1,2,3-triazole-4-carboxamide (80.00 mg, 152.94 μmol, hydrochloride, 1.0 eq.) in DCM (10.00 mL) was added DIPEA (39.53 mg, 305.89 μmol, 2.0 eq.) at 15° C. Then compound acryloyl chloride (16.61 mg, 183.53 μmol, 1.2 eq.) was added to the mixture at 15° C. slowly. Then the mixture was stirred at 15° C. for 1 hour. LCMS showed that the starting material was consumed and the desired product was detected. The mixture was poured into water (50 mL) and extracted with DCM (50 mL×3). The combined organic layers were washed with brine (100 mL), dried over Na₂SO₄, filtered and concentrated under vacuum to give the crude, which was purified by Prep-HPLC (Column: Welch Xtimate C18 150×25 mm×5 □m; Condition: water (10 mM NH₄HCO₃)-ACN, Begin B 34, End B 64, Gradient Time (min) 10, 100% B Hold Time (min) 2, Flow Rate (mL/min) 25.) to give titled compound (42.3 mg, 51.16% yield, 100.00% purity, ee: 100.00%) as white solid. LCMS: m/z=541.4 (M+H⁺). ¹H NMR (400 MHz, DMSO-d6) δ=9.00 (s, 1H), 8.69-8.57 (m, 3H), 7.42-7.30 (m, 3H), 6.68-5.89 (m, 2H), 5.64-5.60 (m, 1H), 4.49-3.48 (m, 3H), 2.98-2.65 (m, 7H), 2.36 (s, 3H), 1.61-1.44 (m, 13H).

### Example 49: N-(4-(3-(4-acryloylpiperazin-1-yl)pyridin-4-yl)-2-methylbenzyl)-1-(tert-butyl)-1H-1,2,3-triazole-4-carboxamide

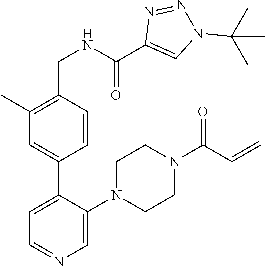 [SMILES: C=CC(=O)N1CCN(c2cnccc2-c2ccc(CNC(=O)c3cn(C(C)(C)C)nn3)c(C)c2)CC1]

### 1. Synthesis of tert-butyl 4-(4-chloropyridin-3-yl)piperazine-1-carboxylate

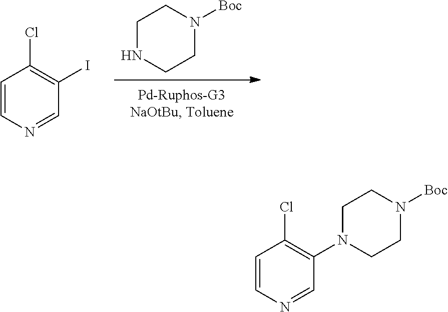 [SMILES: Clc1ccncc1I ; CC(C)(C)OC(=O)N1CCNCC1 ; CC(C)(C)OC(=O)N1CCN(c2cnccc2Cl)CC1]

The mixture of 4-chloro-3-iodopyridine (200.00 mg, 0.835 mmol, 1.0 eq.), tert-butyl piperazine-1-carboxylate (186.69 mg, 1.00 mmol, 1.2 eq.), NaOtBu (240.81 mg, 2.51 mmol, 3.0 eq.) and Pd-Ruphos-G3 (69.86 mg, 0.083 mmol, 0.1 eq.) in toluene (8.00 mL) was bubbled with N₂ for 1 min. The mixture was stirred at 100° C. for 4.5 hours. LCMS showed the starting material was consumed completely and a peak with desired MS was detected. The solvent was removed to get a residue, which was purified by Combi flash eluting with EtOAc in PE from 0% to 30% to give titled compound (150.00 mg, 54.28% yield) as yellow oil. LCMS: m/z=298.1 (M+H⁺).

### 2. Synthesis of tert-butyl 4-(4-(4-((1-(tert-butyl)-1H-1,2,3-triazole-4-carboxamido)methyl)-3-methylphenyl)pyridin-3-yl)piperazine-1-carboxylate

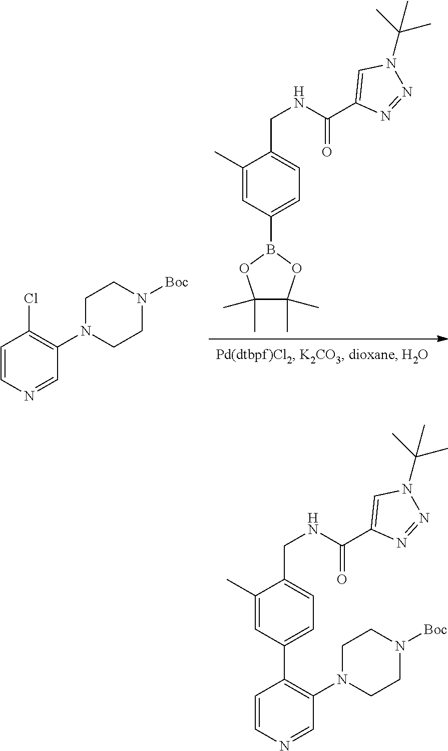 [SMILES: CC(C)(C)OC(=O)N1CCN(c2cnccc2Cl)CC1 ; Cc1cc(B2OC(C)(C)C(C)(C)O2)ccc1CNC(=O)c1cn(C(C)(C)C)nn1 ; Cc1cc(-c2ccncc2N2CCN(C(=O)OC(C)(C)C)CC2)ccc1CNC(=O)c1cn(C(C)(C)C)nn1]

The mixture of tert-butyl 4-(4-chloropyridin-3-yl)piperazine-1-carboxylate (150.00 mg, 0.503 mmol, 1.0 eq.), 1-(tert-butyl)-N-(2-methyl-4-(4,4,5,5-tetramethyl-1,3,2-dioxaborolan-2-yl)benzyl)-1H-1,2,3-triazole-4-carboxamide (220.70 mg, 0.554 mmol, 1.1 eq.), Pd(dtbpf)Cl₂ (32.83 mg, 0.050 mmol, 0.1 eq.) and K₂CO₃ (208.86 mg, 1.51 mmol, 3.0 eq.) in a mixture of dioxane (5.00 mL) and water (1.00 mL) was bubbled with N₂ for 1 min. The mixture was stirred at 90° C. for 16 hours. LCMS showed the starting material was consumed completely and a major peak with desired MS was detected. The solvent was removed to get a residue, which was purified by Si chromatography eluting with EtOAc in PE from 0% to 50% to 100% to give titled compound (260.00 mg, 82.21% yield) as a yellow solid. LCMS: m/z=534.4 (M+H⁺).

### 3. Synthesis of 1-(tert-butyl)-N-(2-methyl-4-(3-(piperazin-1-yl)pyridin-4-yl)benzyl)-1H-1,2,3-triazole-4-carboxamide

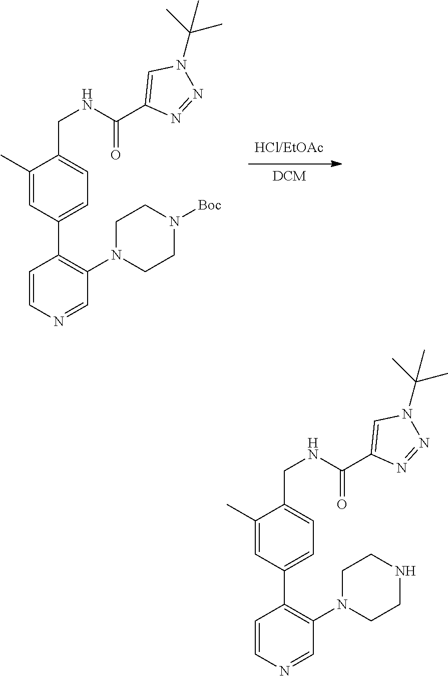

The mixture of tert-butyl 4-(4-(4-((1-(tert-butyl)-1H-1,2,3-triazole-4-carboxamido)methyl)-3-methylphenyl)pyridin-3-yl)piperazine-1-carboxylate (260.00 mg, 0.487 mmol, 1.0 eq.) in DCM (2.00 mL) was added HCl in EtOAc (4 M, 6.00 mL). The mixture was stirred at 25° C. for 1 hour. LCMS showed the starting material was consumed completely and a peak with desired MS were detected. The reaction mixture was concentrated to give titled compound (200.00 mg, crude) as yellow oil, which was used to next step directly. LCMS: m/z=434.3 (M+H⁺).

### 4. Synthesis of N-(4-(3-(4-acryloylpiperazin-1-yl)pyridin-4-yl)-2-methylbenzyl)-1-(tert-butyl)-1H-1,2,3-triazole-4-carboxamide

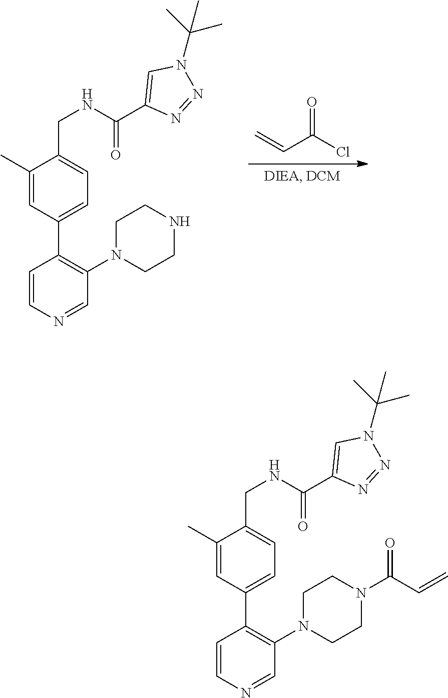

To the mixture of 1-(tert-butyl)-N-(2-methyl-4-(3-(piperazin-1-yl)pyridin-4-yl)benzyl)-1H-1,2,3-triazole-4-carboxamide (200.00 mg, 0.461 mmol, 1.0 eq.) and DIPEA (35.93 mg, 1.38 mmol, 3.0 eq.) in DCM (20.00 mL) was added acryloyl chloride (43.84 mg, 0.484 mmol, 1.05 eq.) at 0° C. The mixture was stirred at 0° C. for 2 min. LCMS showed the starting material was consumed completely and a peak with desired MS were detected. MeOH (2.00 mL) was added dropwise.

The resulting mixture was stirred at 25° C. for 10 min. The solvent was removed to get a crude, which was purified by Prep-HPLC (Column: Agela DuraShell C18 150×25 mm×5 μm; Condition: water (0.05% NH3H2O+10 mM NH₄HCO₃)-ACN, Begin B 29, End B 59, Gradient Time (min) 14, 100% B Hold Time (min) 2, Flow Rate (mL/min) 25) to give titled compound (77.8 mg, 34.59% yield, 100.00% purity) as a pale yellow solid. LCMS: m/z=488.4 (M+H⁺). ¹H NMR (500 MHz, MeOH-d₄) δ ppm=8.50 (s, 1H), 8.28-8.22 (m, 2H), 7.61-7.55 (m, 2H), 7.44 (d, J=8.5 Hz, 1H), 7.30 (d, J=5.0 Hz, 1H), 6.77-6.70 (m, 1H), 6.23-6.18 (m, 1H), 5.76-5.73 (m, 1H), 4.67 (s, 2H), 3.65-3.55 (m, 4H), 2.98-2.89 (m, 4H), 2.47 (s, 3H), 1.73 (s, 9H).

### Example 50: N-(4-(5-(4-acryloylpiperazin-1-yl)pyrimidin-4-yl)-2-methylbenzyl)-1-(tert-butyl)-1H-1,2,3-triazole-4-carboxamide

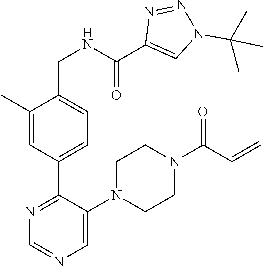 [SMILES: C=CC(=O)N1CCN(c2cncnc2-c2ccc(CNC(=O)c3cn(C(C)(C)C)nn3)c(C)c2)CC1]

### 1. Synthesis of tert-butyl 4-(4-(tert-butoxy)pyrimidin-5-yl)piperazine-1-carboxylate

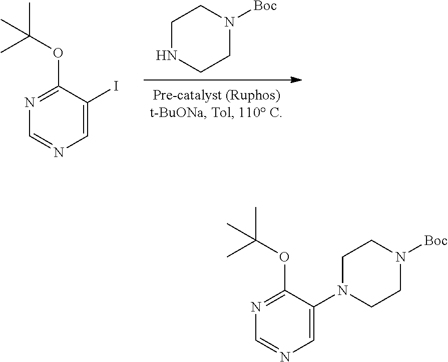 [SMILES: CC(C)(C)Oc1ncncc1I ; CC(C)(C)OC(=O)N1CCNCC1 ; CC(C)(C)OC(=O)N1CCN(c2cncnc2OC(C)(C)C)CC1]

The mixture of 4-(tert-butoxy)-5-iodopyrimidine (400.00 mg, 1.44 mmol, 1.0 eq.), tert-butyl piperazine-1-carboxylate (321.84 mg, 1.73 mmol, 1.2 eq.), t-BuONa (276.77 mg, 2.88 mmol, 2.0 eq.) and Pre-catalyst (Ruphos) (120.44 mg, 144.00 μmol, 0.1 eq.) in toluene (15.00 mL) was bubbled with N₂ for 1 min. The reaction mixture was stirred at 90° C. under N₂ for 6 h. LCMS showed the reaction was completed. The reaction mixture was concentrated in vacuum to give crude product, which was purified by silica gel chromatography (PE/EA=10/1 to 3/1) to give titled compound (456.00 mg, 84.72% yield) as a brown solid. LCMS: m/z=337.3 (M+H⁺).

### 2. Synthesis of tert-butyl 4-(4-hydroxypyrimidin-5-yl)piperazine-1-carboxylate

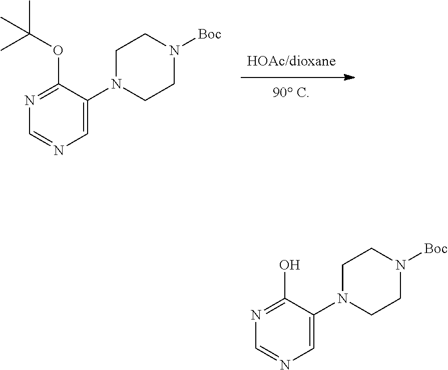 [SMILES: CC(C)(C)OC(=O)N1CCN(c2cncnc2OC(C)(C)C)CC1 ; CC(C)(C)OC(=O)N1CCN(c2cncnc2O)CC1]

The mixture of tert-butyl 4-(4-(tert-butoxy)pyrimidin-5-yl)piperazine-1-carboxylate (456.00 mg, 1.36 mmol, 1.0 eq.) in HOAc (10.00 mL) and dioxane (10.00 mL) was stirred at 90° C. for 2 h. LCMS showed several peaks were detected and a peak with desired MS was found. The reaction was concentrated to give a crude product, which was adjust to pH >7 by adding Na₂CO₃ aqueous solution. The reaction was concentrated to give a crude product, which was purified by column chromatography (DCM: MeOH=10:1) to give titled compound (350.00 mg, 91.81% yield.) as a yellow solid. LCMS: m/z=281.3 (M+H⁺).

### 3. Synthesis of tert-butyl 4-(4-(4-((1-(tert-butyl)-1H-1,2,3-triazole-4-carboxamido)methyl)-3-methylphenyl)pyrimidin-5-yl)piperazine-1-carboxylate

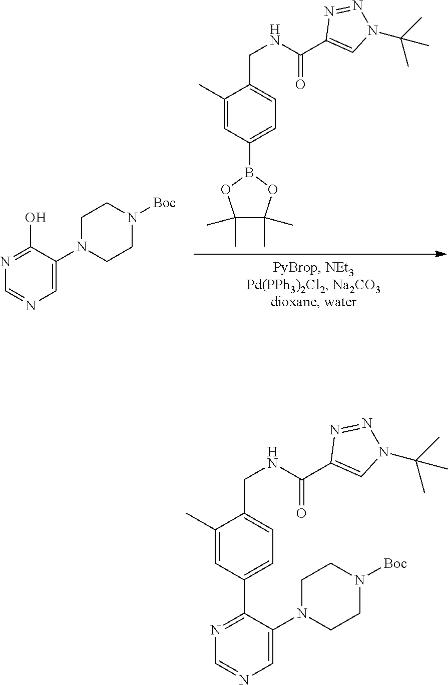 [SMILES: CC(C)(C)OC(=O)N1CCN(c2cncnc2O)CC1 ; Cc1cc(B2OC(C)(C)C(C)(C)O2)ccc1CNC(=O)c1cn(C(C)(C)C)nn1 ; Cc1cc(-c2ncncc2N2CCN(C(=O)OC(C)(C)C)CC2)ccc1CNC(=O)c1cn(C(C)(C)C)nn1]

To a solution of tert-butyl 4-(4-hydroxypyrimidin-5-yl)piperazine-1-carboxylate (150.00 mg, 535.10 μmol, 1.0 eq.) in dioxane (3.00 mL) was added TEA (162.44 mg, 1.61 mmol, 3.0 eq.) and PyBrop (249.45 mg, 535.10 μmol, 1.0 eq.). The mixture was stirred at 20° C. for 2 hours. Then 1-(tert-butyl)-N-(2-methyl-4-(4,4,5,5-tetramethyl-1,3,2-dioxaborolan-2-yl)benzyl)-1H-1,2,3-triazole-4-carboxamide (234.45 mg, 588.61 μmol, 1.1 eq.), Na₂CO₃ (170.15 mg, 1.61 mmol, 3.0 eq.), Pd(PPh₃)₂Cl₂ (37.56 mg, 53.51 μmol, 0.1 eq.) and water (500.17 μL) was added into the mixture and stirred at 90° C. under N₂ for 8 hours. LCMS showed the starting material was consumed and desired MS was detected. The solvent was removed under vacuum to give a residue. The residue was purified by silica gel chromatography (from PE to EA) to give titled compound (200.00 mg, 55.93% yield) as yellow oil. LCMS: m/z=535.3 (M+H⁺).

### 4. Synthesis of 1-(tert-butyl)-N-(2-methyl-4-(5-(piperazin-1-yl)pyrimidin-4-yl)benzyl)-1H-1,2,3-triazole-4-carboxamide

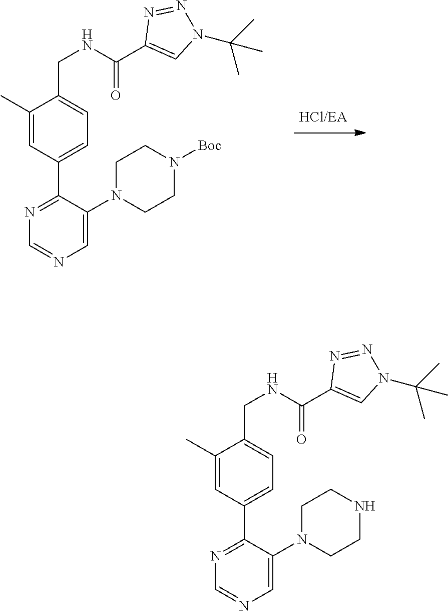 [SMILES: Cc1cc(-c2ncncc2N2CCN(C(=O)OC(C)(C)C)CC2)ccc1CNC(=O)c1cn(C(C)(C)C)nn1 ; Cc1cc(-c2ncncc2N2CCNCC2)ccc1CNC(=O)c1cn(C(C)(C)C)nn1]

The mixture of tert-butyl 4-(4-(4-((1-(tert-butyl)-1H-1,2,3-triazole-4-carboxamido)methyl)-3-methylphenyl)pyrimidin-5-yl)piperazine-1-carboxylate (200.00 mg, 374.08 μmol, 1.0 eq.) in HCl/EA (4M, 15.00 mL) was stirred at 9° C. for 30 min. LCMS showed the starting material was consumed completely. The crude material was concentrated under vacuum to give titled compound (115.00 mg, crude.) as yellow oil. LCMS: m/z=435.3 (M+H⁺).

### 5. Synthesis of N-(4-(5-(4-acryloylpiperazin-1-yl)pyrimidin-4-yl)-2-methylbenzyl)-1-(tert-butyl)-1H-1,2,3-triazole-4-carboxamide

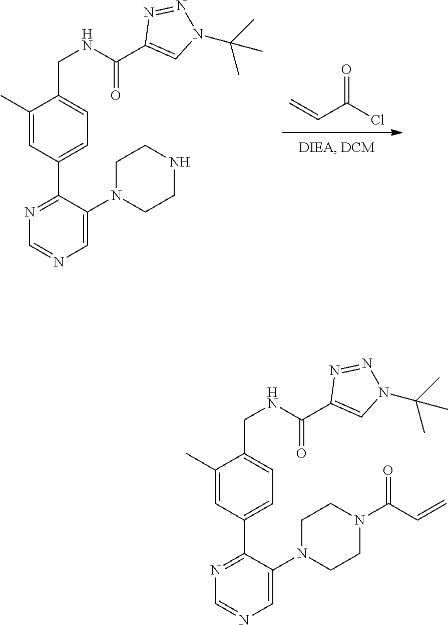

To a solution of 1-(tert-butyl)-N-(2-methyl-4-(5-(piperazin-1-yl)pyrimidin-4-yl)benzyl)-1H-1,2,3-triazole-4-carboxamide (115.00 mg, 264.65 μmol, 1.0 eq.) and DIPEA (68.41 mg, 529.30 mol, 2.0 eq.) in DCM (20.00 mL) was added dropwise acryloyl chloride (26.35 mg, 291.11 mol, 1.1 eq.) at 0° C. The mixture was stirred at 0° C. for 5 min. LCMS showed the starting material was consumed completely. MeOH (6 mL) was added dropwise. The resulting mixture was stirred at 8° C. for 10 min. The solvent was removed to get a residue, which was purified by Prep HPLC (Column: Welch Xtimate C18 150×25 mm×5 μm; Condition water (10 mM NH₄HCO₃)-ACN, Begin B 23, End B 53, Gradient Time (min) 10, 100% B Hold, Time (min) 2, Flow Rate (mL/min) 25) to give titled compound (41.10 mg, 31.79% yield, 100.00% purity) as a white solid. LCMS: m/z=489.4 (M+H⁺). ¹H NMR (500 MHz, MeOH-d₄) δ=8.83 (s, 1H), 8.50 (s, 2H), 7.95-7.90 (m, 2H), 7.47 (d, J=8.0 Hz, 1H), 6.75 (dd, J=10.5, 17.0 Hz, 1H), 6.22 (dd, J=2.0, 16.5 Hz, 1H), 5.76 (dd, J₁=2.0, J2=10.5 Hz, 1H), 4.69 (s, 2H), 3.68 (s, 4H), 3.01-2.98 (m, 4H), 2.49 (s, 3H), 1.73 (s, 9H).

### Example 51: (R)-1-(tert-butyl)-N-(2-methyl-4-(3-(3-(N-methylacrylamido)azepan-1-yl)pyridin-4-yl)benzyl)-1H-1,2,3-triazole-4-carboxamide

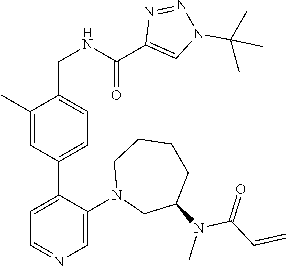 [SMILES: C=CC(=O)N(C)[C@@H]1CCCCN(c2cnccc2-c2ccc(CNC(=O)c3cn(C(C)(C)C)nn3)c(C)c2)C1]

### 1. Synthesis of tert-butyl (R)-3-(((benzyloxy)carbonyl)amino)azepane-1-carboxylate

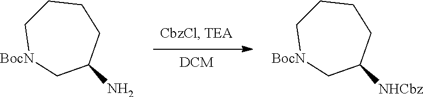

To a solution of tert-butyl (R)-3-aminoazepane-1-carboxylate (800.00 mg, 3.73 mmol, 1.0 eq.) in DCM (50.00 mL) was added TEA (755.50 mg, 7.47 mmol, 2.0 eq.) and the mixture was cooled to −70° C. Then CbzCl (955.24 mg, 5.60 mmol, 1.5 eq.) was added into the mixture and the mixture was stirred at −70° C. for 30 min. LCMS showed product mass was observed. The reaction mixture was concentrated under vacuum to give a crude, which was purified by column chromatography (PE/EA=7/3) to give titled compound (1.20 g, 83.10% yield) as clear oil. LCMS: m/z=249.2 (M+H⁺-Boc).

### 2. Synthesis of tert-butyl (R)-3-(((benzyloxy)carbonyl)(methyl)amino)azepane-1-carboxylate

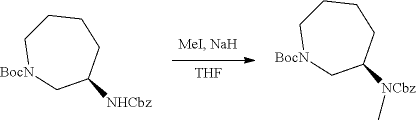 [SMILES: CC(C)(C)OC(=O)N1CCCC[C@@H](NC(=O)OCc2ccccc2)C1 ; CN(C(=O)OCc1ccccc1)[C@@H]1CCCCN(C(=O)OC(C)(C)C)C1 ; "THF"]

To a solution of tert-butyl (R)-3-(((benzyloxy)carbonyl)amino)azepane-1-carboxylate (1.20 g, 3.44 mmol, 1.0 eq.) in THF (50.00 mL) was added NaH (688.78 mg, 17.22 mmol, 5.0 eq.) and the mixture was stirred at 0° C. for 30 min. Then MeI (3.47 g, 24.45 mmol, 7.11 eq.) was added to the mixture and the mixture was stirred at 20° C. for 1 hour. The appliance was quenched with NH₃H₂O. LCMS showed product mass was observed. The mixture was quenched with MeOH (20.00 mL). The reaction mixture was concentrated under vacuum to give a crude, which was purified by column chromatography (PE/EA=4/1) to give titled compound (1.20 g, 89.50% yield) as clear oil. LCMS: m/z=363.3 (M+H⁺).

### 3. Synthesis of benzyl (R)-azepan-3-yl(methyl)carbamate

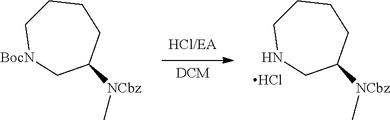

To a solution of tert-butyl (R)-3-(((benzyloxy)carbonyl)(methyl)amino)azepane-1-carboxylate (1.20 g, 3.31 mmol, 1.0 eq.) in DCM (20.00 mL) was added HCl/EA (4 M, 4.00 mL) and the mixture was stirred at 20° C. for 30 min. LCMS showed product mass was observed. The mixture was concentrated under vacuum to give titled compound (980.00 mg, crude, Hydrochloride) as a white solid. It was used for the next step without purification. LCMS: m/z=263.2 (M+H⁺).

### 4. Synthesis of benzyl (R)-(1-(4-chloropyridin-3-yl)azepan-3-yl)(methyl)carbamate

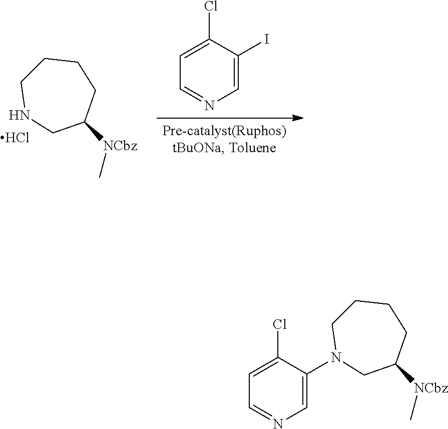

To a solution of benzyl (R)-azepan-3-yl(methyl)carbamate (500.00 mg, 1.67 mmol, Hydrochloride, 1.0 eq.) and 4-chloro-3-iodopyridine (479.84 mg, 2.00 mmol, 1.2 eq.) in toluene (15.00 mL) was added t-BuONa (481.46 mg, 5.01 mmol, 3.0 eq.). Then Pre-catalyst(Ruphos) (139.67 mg, 167.00 μmol, 0.1 eq.) was added into the mixture and the mixture was stirred at 100° C. under N₂ for 2 hours. LCMS showed product mass was observed. The reaction mixture was concentrated under vacuum to give a crude, which was purified by column chromatography (PE/EA=1/1) to give titled compound (100.00 mg, 14.41% yield) as brown oil. LCMS: m/z=374.2 (M+H⁺).

### 5. (R)-1-(4-chloropyridin-3-yl)-N-methylazepan-3-amine

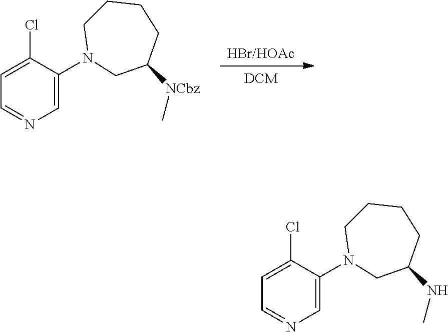

To a solution of benzyl (R)-(1-(4-chloropyridin-3-yl)azepan-3-yl)(methyl)carbamate (100.00 mg, 267.47 μmol, 1.0 eq.) in DCM (10.00 mL) was added HBr/HOAc (1.00 mL, 33 wt %) and the mixture was stirred at 20° C. for 30 min. LCMS showed product mass was observed. The mixture was concentrated under vacuum to give titled compound (60.00 mg, crude) as orange oil. It was used for the next step without purification. LCMS: m/z=240.1 (M+H⁺).

### 6. Synthesis of tert-butyl (R)-(1-(4-chloropyridin-3-yl)azepan-3-yl)(methyl)carbamate

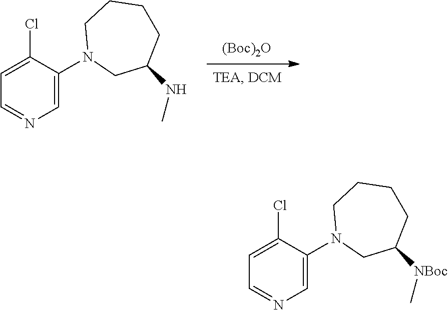 [SMILES: "(Boc)2O" ; CN[C@@H]1CCCCN(c2cnccc2Cl)C1 ; CN(C(=O)OC(C)(C)C)[C@@H]1CCCCN(c2cnccc2Cl)C1]

To a solution of (R)-1-(4-chloropyridin-3-yl)-N-methylazepan-3-amine (60.00 mg, 250.27 μmol, 1.0 eq.) in DCM (20.00 mL) was added TEA (75.97 mg, 750.81 μmol, 3.0 eq.). Then (Boc)₂O (109.24 mg, 500.54 μmol, 2.0 eq.) was added into the mixture and the mixture was stirred at 20° C. for 30 min. LCMS showed product mass was observed. The reaction mixture was concentrated under vacuum to give a crude, which was purified by column chromatography (PE/EA=7/3) to give titled compound (80.00 mg, 89.35% yield) as brown oil. LCMS: m/z=340.2 (M+H⁺).

### 7. tert-butyl (R)-(1-(4-(4-((1-(tert-butyl)-1H-1,2,3-triazole-4-carboxamido)methyl)-3-methylphenyl)pyridin-3-yl)azepan-3-yl)(methyl)carbamate

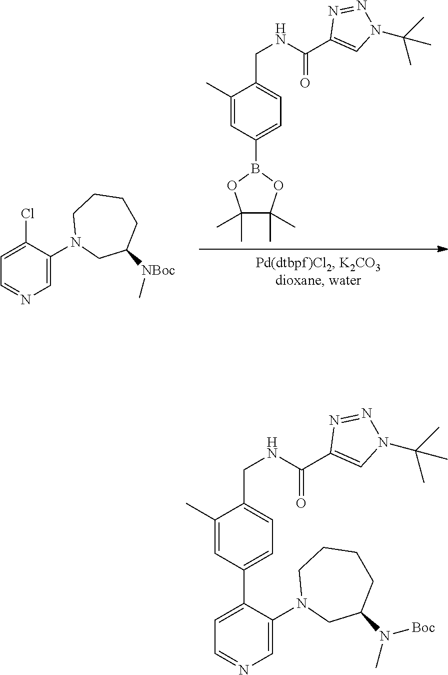 [SMILES: CN(C(=O)OC(C)(C)C)[C@@H]1CCCCN(c2cnccc2Cl)C1 ; Cc1cc(B2OC(C)(C)C(C)(C)O2)ccc1CNC(=O)c1cn(C(C)(C)C)nn1 ; Cc1cc(-c2ccncc2N2CCCC[C@@H](N(C)C(=O)OC(C)(C)C)C2)ccc1CNC(=O)c1cn(C(C)(C)C)nn1]

To a solution of tert-butyl (R)-(1-(4-chloropyridin-3-yl)azepan-3-yl)(methyl)carbamate (70.00 mg, 205.97 μmol, 1.0 eq.) and 1-(tert-butyl)-N-(2-methyl-4-(4,4,5,5-tetramethyl-1,3,2-dioxaborolan-2-yl)benzyl)-1H-1,2,3-triazole-4-carboxamide (98.45 mg, 247.16 μmol, 1.2 eq.) in dioxane (10.00 mL) was added water (999.89 uL). Then K₂CO₃ (85.40 mg, 617.90 μmol, 3.0 eq.) and Pd(dtbpf)Cl₂ (13.42 mg, 20.60 μmol, 0.1 eq.) was added into the mixture and the mixture was stirred at 80° C. under N₂ for 2 hours. LCMS showed product mass was observed. The reaction mixture was concentrated under vacuum to give a crude, which was purified by column chromatography (PE/EA=1/4) to give titled compound (80.00 mg, 49.25% yield) as brown oil. LCMS: m/z=576.4 (M+H⁺).

### 8. (R)-1-(tert-butyl)-N-(2-methyl-4-(3-(3-(methylamino)azepan-1-yl)pyridin-4-yl)benzyl)-1H-1,2,3-triazole-4-carboxamide

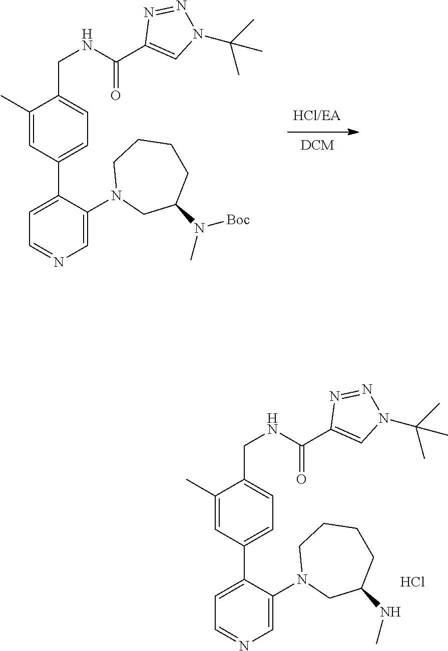

A solution of tert-butyl (R)-(1-(4-(4-((1-(tert-butyl)-1H-1,2,3-triazole-4-carboxamido)methyl)-3-methylphenyl)pyridin-3-yl)azepan-3-yl)(methyl)carbamate (80.00 mg, 138.95 μmol, 1.0 eq.) in HCl/EA (4 M, 10.00 mL) and the mixture was stirred at 20° C. for 30 min. LCMS showed product mass was observed. The mixture was concentrated under vacuum to give titled compound (70.00 mg, crude, Hydrochloride) as a yellow solid. It was used for the next step without purification. LCMS: m/z=476.4 (M+H⁺).

### 9. Synthesis of (R)-1-(tert-butyl)-N-(2-methyl-4-(3-(3-(N-methylacrylamido)azepan-1-yl)pyridin-4-yl)benzyl)-1H-1,2,3-triazole-4-carboxamide

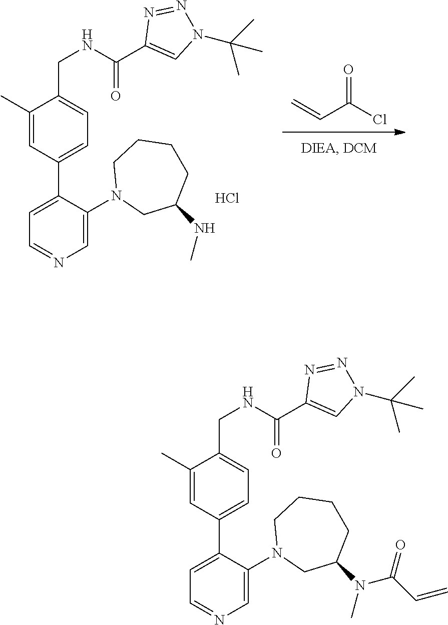

To a solution of (R)-1-(tert-butyl)-N-(2-methyl-4-(3-(3-(methylamino)azepan-1-yl)pyridin-4-yl)benzyl)-1H-1,2,3-triazole-4-carboxamide (65.00 mg, 126.93 μmol, Hydrochloride, 1.0 eq.) in DCM (50.00 mL) was added DIPEA (49.21 mg, 380.79 μmol, 3.0 eq.). Then acryloyl chloride (13.79 mg, 152.32 μmol, 1.2 eq.) was added into the mixture and the mixture was stirred at 20° C. for 30 min. LCMS showed product mass was observed. The mixture was concentrated under vacuum to give a brown crude, which was purified by Prep-HPLC (Column: Welch Xtimate C18 150×25 mm×5 □m; Condition: water (10 mM NH₄HCO₃)-ACN, Begin B 35, End B 65, Gradient Time (min) 10, 100% B Hold Time (min) 2, Flow Rate (mL/min) 25.) to give titled product (36.00 mg, 52.42% yield) as a yellow solid. LCMS: m/z=530.3 (M+H⁺). ¹H NMR (400 MHz, DMSO-d6) δ ppm=8.94 (s, 1H), 8.70 (s, 1H), 8.49-8.43 (m, 1H), 8.21-8.13 (m, 1H), 7.36-7.22 (m, 3H), 7.12-7.05 (m, 1H), 6.67-6.59 (m, 1H), 6.11-5.98 (m, 1H), 5.66-5.58 (m, 1H), 4.54-3.85 (m, 3H), 3.33-3.10 (m, 3H), 3.03-2.90 (m, 1H), 2.76-2.56 (m, 3H), 2.36 (s, 3H), 1.63 (s, 11H), 1.57-1.26 (m, 4H).

### Example 52: 1-(tert-butyl)-N-(2-methyl-4-(3-(6-(N-methylacrylamido)-1,4-oxazepan-4-yl)pyridin-4-yl)benzyl)-1H-1,2,3-triazole-4-carboxamide

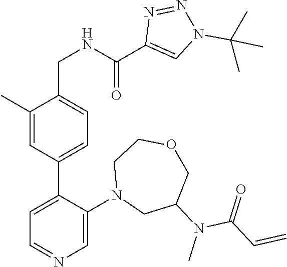 [SMILES: C=CC(=O)N(C)C1COCCN(c2cnccc2-c2ccc(CNC(=O)c3cn(C(C)(C)C)nn3)c(C)c2)C1]

### 1. Synthesis of tert-butyl 6-methylene-1,4-oxazepane-4-carboxylate

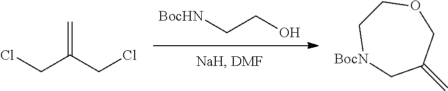

To a solution of 3-chloro-2-(chloromethyl)prop-1-ene (5.00 g, 40.00 mmol, 1.0 eq.) in DMF (80.00 mL) was added NaH (3.63 g, 90.80 mmol, 60% purity, 2.27 eq.) at 0° C. The mixture was stirred at 0° C. for 30 min. Then tert-butyl (2-hydroxyethyl)carbamate (6.13 g, 38.00 mmol, 0.95 eq.) was added at 0° C. The mixture was stirred at 20° C. for 2 hours. LCMS showed product mass was observed. Saturated NH4Cl (300 mL) was added to quench the reaction. The solvent was removed in vacuum and the residue was extracted with EA (200 mL×2). The organic layer was concentrated and purified by silica gel column (PE/EA=4/1) to give titled compound (4.30 g, 50.41% yield) as colorless oil. LCMS: m/z=158.1 (M+H⁺−56).

### 2. Synthesis of tert-butyl 6-oxo-1,4-oxazepane-4-carboxylate

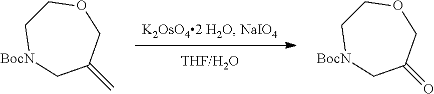

To a solution of tert-butyl 6-methylene-1,4-oxazepane-4-carboxylate (4.30 g, 20.16 mmol, 1.0 eq.) in THF (60.00 mL) was added water (60.00 mL). Then K₂OsO₄·2 (H₂O) (371.45 mg, 1.01 mmol, 0.05 eq.) and NaIO₄ (9.96 g, 46.57 mmol, 2.31 eq.) was added to the mixture and the mixture was stirred at 20° C. for 4 hours. LCMS showed product mass was observed. The mixture was poured EA (30 mL) and filtered to remove the solid. The mixture was poured into H₂O (30 mL) and extracted with EA (30 mL×2). The combined organic layers were washed with brine (40 mL), dried over Na₂SO₄, and concentrated under vacuum to give the crude, which was purified by chromatography column on silica gel (PE/EA=5/1) to give titled compound (4.15 g, 86.07% yield) as yellow oil. LCMS: m/z=160.1 (M+H⁺−56).

### 3. Synthesis of tert-butyl 6-(methylamino)-1,4-oxazepane-4-carboxylate

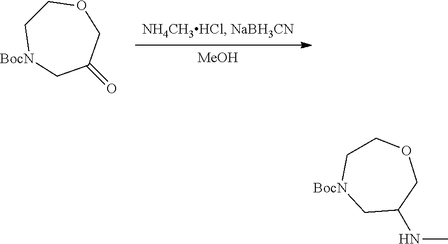 [SMILES: "MeOH" ; CC(C)(C)OC(=O)N1CCOCC(=O)C1 ; CNC1COCCN(C(=O)OC(C)(C)C)C1]

To a solution of tert-butyl 6-oxo-1,4-oxazepane-4-carboxylate (4.15 g, 19.28 mmol, 1.0 eq.) in MeOH (150.00 mL) was added methanamine; hydrochloride (13.02 g, 192.80 mmol, 10.0 eq.) and the mixture was stirred at 60° C. for 2 hours. Then sodium cyanoboranuide (6.06 g, 96.40 mmol, 5.0 eq.) was added into the mixture and the mixture was stirred at 60° C. for 16 hours. LCMS showed product mass was observed. The mixture was concentrated under vacuum to give a crude, which was poured into water (100 mL) and extracted with EA (50 mL×3). The combined organic layers were washed with brine (50 mL), dried over Na₂SO₄, filtered and concentrated under vacuum to give titled compound (4.40 g, crude) as yellow oil. It was used for the next step without further purification. LCMS: m/z=231.2 (M+H⁺).

### 4. Synthesis of tert-butyl 6-(((benzyloxy)carbonyl)(methyl)amino)-1,4-oxazepane-4-carboxylate

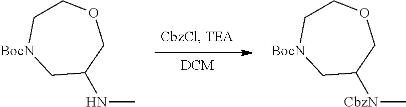

To a solution of tert-butyl 6-(methylamino)-1,4-oxazepane-4-carboxylate (4.40 g, 19.11 mmol, 1.0 eq.) in DCM (150.00 mL) was added TEA (3.87 g, 38.22 mmol, 2.0 eq.). Then CbzCl (4.89 g, 28.66 mmol, 1.5 eq.) was added into the mixture and the mixture was stirred at 0° C. for 30 min. LCMS showed product mass was observed. The reaction mixture was concentrated under vacuum to give a crude, which was purified by column chromatography (PE/EA=4/1) to give titled compound (2.52 g, 34.38% yield) as clear oil. LCMS: m/z=265.2 (M-Boc+H⁺).

### 5. Synthesis of benzyl methyl(1,4-oxazepan-6-yl)carbamate

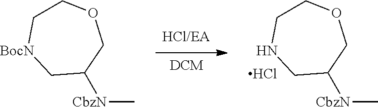

To a solution of tert-butyl 6-(((benzyloxy)carbonyl)(methyl)amino)-1,4-oxazepane-4-carboxylate (520.00 mg, 1.43 mmol, 1.0 eq.) in DCM (20.00 mL) was added HCl/EA (4 M, 4.00 mL) and the mixture was stirred at 20° C. for 3 hours. LCMS showed product mass was observed. The mixture was concentrated under vacuum to give titled compound (430.00 mg, crude, Hydrochloride) as clear oil. It was used for the next step without further purification. LCMS: m/z=265.3 (M+H⁺).

### 6. Synthesis of benzyl (4-(4-chloropyridin-3-yl)-1,4-oxazepan-6-yl)(methyl)carbamate

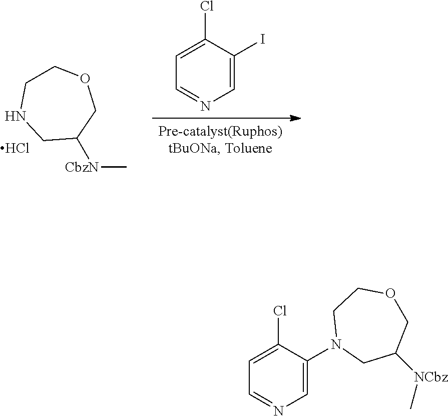

To a solution of benzyl methyl(1,4-oxazepan-6-yl)carbamate (430.00 mg, 1.43 mmol, Hydrochloride, 1.0 eq.) and 4-chloro-3-iodopyridine (376.54 mg, 1.57 mmol, 1.1 eq.) in Toluene (12.00 mL) was added tBuONa (412.16 mg, 4.29 mmol, 3.0 eq.). Then Pre-catalyst (Ruphos) (119.57 mg, 143.00 μmol, 0.1 eq.) was added to the mixture and the mixture was stirred at 100° C. under N₂ for 2 hours. LCMS showed product mass was observed. The reaction mixture was concentrated under vacuum to give a crude, which was purified by column chromatography (PE/EA=1/1) to give titled compound (180.00 mg, 29.47% yield) as brown oil. LCMS: m/z=376.1 (M+H⁺).

### 7. Synthesis of 4-(4-chloropyridin-3-yl)-N-methyl-1,4-oxazepan-6-amine

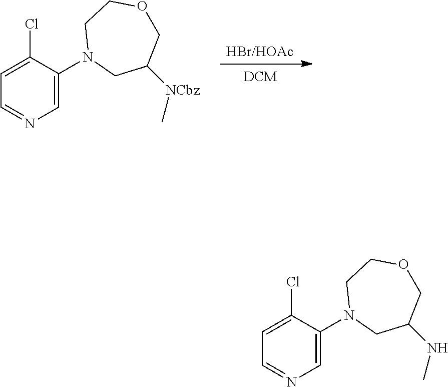

To solution of benzyl (4-(4-chloropyridin-3-yl)-1,4-oxazepan-6-yl)(methyl)carbamate (180.00 mg, 478.91 μmol, 1.0 eq.) in DCM (10.00 mL) was added HBr/HOAc (1.00 mL, 33% purity) and the mixture was stirred at 20° C. for 30 min. LCMS showed product mass was observed.

The mixture was concentrated under vacuum to give titled compound (110.00 mg, crude) as orange oil. It was used for the next step without further purification. LCMS: m/z=242.4 (M+H⁺).

### 8. Synthesis of tert-butyl (4-(4-chloropyridin-3-yl)-1,4-oxazepan-6-yl)(methyl)carbamate

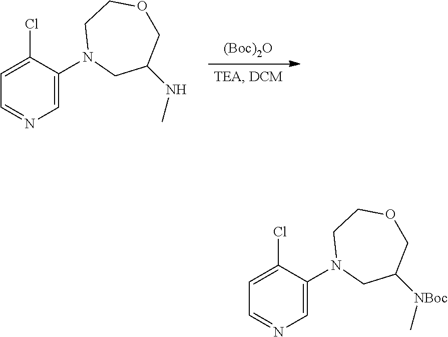 [SMILES: "(Boc)2O" ; CNC1COCCN(c2cnccc2Cl)C1 ; "TEA, DCM" ; CN(C(=O)OC(C)(C)C)C1COCCN(c2cnccc2Cl)C1]

To a solution of 4-(4-chloropyridin-3-yl)-N-methyl-1,4-oxazepan-6-amine (110.00 mg, 455.07 mol, 1.0 eq.) in DCM (30.00 mL) was added TEA (138.15 mg, 1.37 mmol, 3.0 eq.). Then (Boc)₂₀ (198.64 mg, 910.14 μmol, 2.0 eq.) was added into the mixture and the mixture was stirred at 20° C. for 30 min. LCMS showed product mass was observed. The reaction mixture was concentrated under vacuum to give a crude, which was purified by column chromatography (PE/EA=7/3) to give titled compound (140.00 mg, 81.00% yield) as brown oil. LCMS: m/z 342.1 (M+H⁺).

### 9. Synthesis of tert-butyl (4-(4-(4-((1-(tert-butyl)-1H-1,2,3-triazole-4-carboxamido)methyl)-3-methylphenyl)pyridin-3-yl)-1,4-oxazepan-6-yl)(methyl)carbamate

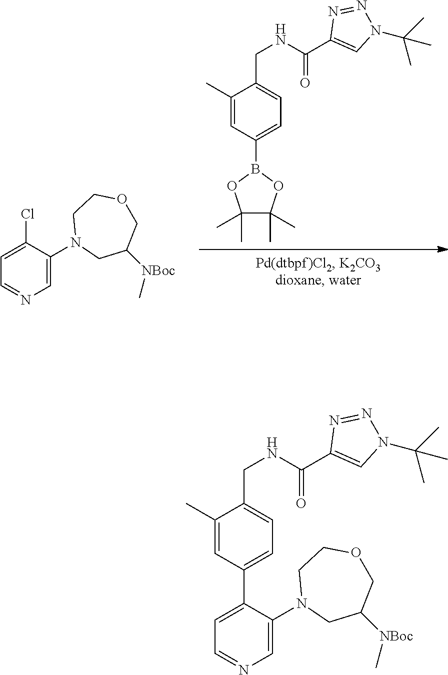 [SMILES: CN(C(=O)OC(C)(C)C)C1COCCN(c2cnccc2Cl)C1 ; Cc1cc(B2OC(C)(C)C(C)(C)O2)ccc1CNC(=O)c1cn(C(C)(C)C)nn1 ; Cc1cc(-c2ccncc2N2CCOCC(N(C)C(=O)OC(C)(C)C)C2)ccc1CNC(=O)c1cn(C(C)(C)C)nn1]

To a solution of tert-butyl (4-(4-chloropyridin-3-yl)-1,4-oxazepan-6-yl)(methyl)carbamate (130.00 mg, 380.31 μmol, 1.0 eq.) and 1-(tert-butyl)-N-(2-methyl-4-(4,4,5,5-tetramethyl-1,3,2-dioxaborolan-2-yl)benzyl)-1H-1,2,3-triazole-4-carboxamide (151.48 mg, 380.31 μmol, 1.0 eq.) in Dioxane (10.00 mL) was added water (999.89 uL). Then K₂CO₃ (157.69 mg, 1.14 mmol, 3.0 eq.) and Pd(dtbpf)Cl₂ (24.79 mg, 38.03 μmol, 0.1 eq.) were added into the mixture and the mixture was stirred at 85° C. under N₂ for 2 hours. LCMS showed product mass was observed.

The reaction mixture was concentrated under vacuum to give a crude, which was purified by column chromatography (PE/EA=1/4) to give titled compound (115.00 mg, 47.11% yield) as brown oil. LCMS: m/z=578.4 (M+H⁺).

### 10. Synthesis of 1-(tert-butyl)-N-(2-methyl-4-(3-(6-(methylamino)-1,4-oxazepan-4-yl)pyridin-4-yl)benzyl)-1H-1,2,3-triazole-4-carboxamide

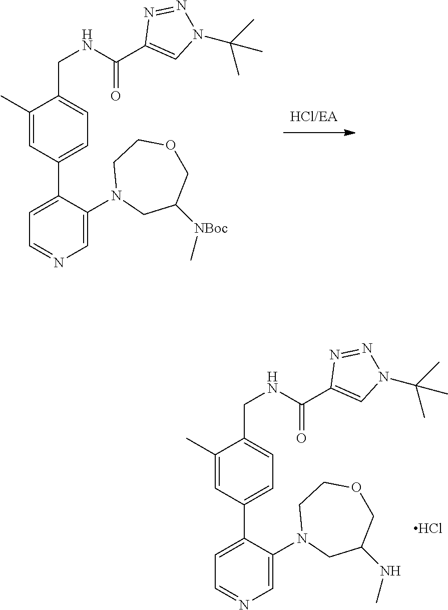 [SMILES: Cc1cc(-c2ccncc2N2CCOCC(N(C)C(=O)OC(C)(C)C)C2)ccc1CNC(=O)c1cn(C(C)(C)C)nn1 ; CNC1COCCN(c2cnccc2-c2ccc(CNC(=O)c3cn(C(C)(C)C)nn3)c(C)c2)C1 ; Cl]

A solution of tert-butyl (4-(4-(4-((1-(tert-butyl)-1H-1,2,3-triazole-4-carboxamido)methyl)-3-methylphenyl)pyridin-3-yl)-1,4-oxazepan-6-yl)(methyl)carbamate (115.00 mg, 199.06 μmol, 1.0 eq.) in HCl/EA (4 M, 20.00 mL) was stirred at 20° C. for 30 min. LCMS showed product mass was observed. The mixture was concentrated under vacuum to give titled compound (95.00 mg, crude) as a yellow solid. It was used for the next step without further purification. LCMS: m/z=478.3 (M+H⁺).

### 11. Synthesis of 1-(tert-butyl)-N-(2-methyl-4-(3-(6-(N-methylacrylamido)-1,4-oxazepan-4-yl)pyridin-4-yl)benzyl)-1H-1,2,3-triazole-4-carboxamide

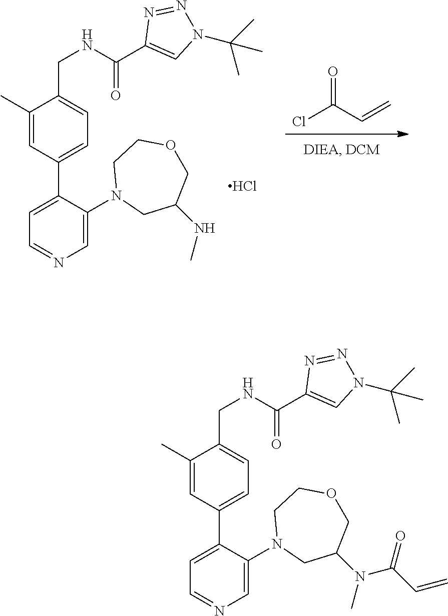

To a solution of 1-(tert-butyl)-N-(2-methyl-4-(3-(6-(methylamino)-1,4-oxazepan-4-yl)pyridin-4-yl)benzyl)-1H-1,2,3-triazole-4-carboxamide (95.00 mg, 198.91 μmol, 1.0 eq.) in DCM (50.00 mL) was added DIPEA (77.12 mg, 596.73 μmol, 3.0 eq.). Then acryloyl chloride (21.60 mg, 238.69 μmol, 1.2 eq.) was added into the mixture and the mixture was stirred at 20° C. for 30 min. LCMS showed product mass was observed. The mixture was concentrated under vacuum to give a brown crude, which was purified by Prep-HPLC (Column: Welch Xtimate C18 150×25 mm×5 Dim; Condition: water (10 mM NH₄HCO₃)-ACN, Begin B 27, End B 57, Gradient Time (min) 10, 100% B Hold Time (min) 2, Flow Rate (mL/min) 25.) to give titled compound (54.00 mg, 51.06% yield, 100.00% purity) as a yellow solid. LCMS: m/z=532.5 (M+H⁺). ¹H NMR: (400 MHz, DMSO-d6) δ ppm=9.00-8.90 (m, 1H), 8.68 (d, J=10.4 Hz, 1H), 8.45 (d, J=7.6 Hz, 1H), 8.25-8.21 (m, 1H), 7.48-7.27 (m, 3H), 7.18-7.12 (m, 1H), 6.65-6.04 (m, 1H), 5.96-5.85 (m, 1H), 5.67-5.48 (m, 1H), 4.48 (d, J=6.4 Hz, 2H), 3.89-3.80 (m, 1H), 3.78-3.36 (m, 4H), 3.29-3.01 (m, 4H), 2.89-2.73 (m, 3H), 2.39 (d, J=14.8 Hz, 3H), 1.63 (s, 9H).

### Example 53: (S)-2-(tert-butyl)-N-(2-methyl-4-(3-(5-(N-methylacrylamido)-2-oxopiperidin-1-yl)pyridin-4-yl)benzyl)-2H-tetrazole-5-carboxamide and (R)-2-(tert-butyl)-N-(2-methyl-4-(3-(5-(N-methylacrylamido)-2-oxopiperidin-1-yl)pyridin-4-yl)benzyl)-2H-tetrazole-5-carboxamide

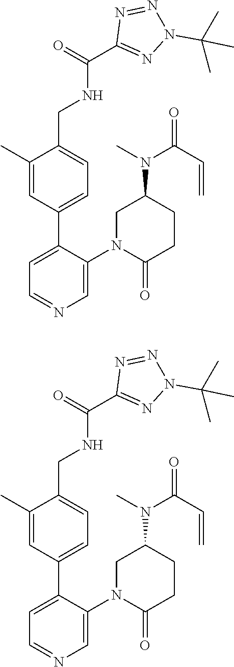

### Synthesis of tert-butyl (1-(4-chloropyridin-3-yl)-6-oxopiperidin-3-yl)carbamate

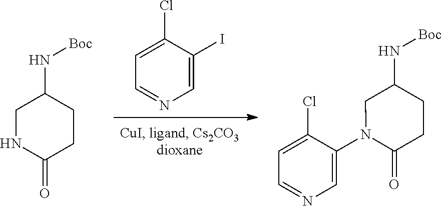 [SMILES: CC(C)(C)OC(=O)NC1CCC(=O)NC1 ; Clc1ccncc1I ; CC(C)(C)OC(=O)NC1CCC(=O)N(c2cnccc2Cl)C1]

To a solution of 4-chloro-3-iodopyridine (1.00 g, 4.18 mmol, 1.0 eq.) in dioxane (15.00 mL) was added tert-butyl (6-oxopiperidin-3-yl)carbamate (894.84 mg, 4.18 mmol, 1.0 eq.), Cs₂CO₃ (4.08 g, 12.53 mmol, 3.0 eq.), CuI (318.16 mg, 1.67 mmol, 0.4 eq.) and (1R,2R)-N₁,N₂-dimethylcyclohexane-1,2-diamine (237.62 mg, 1.67 mmol, 0.4 eq.) at 15° C. The mixture was stirred at 110° C. under N₂ for 10 hours. LCMS showed the desired MS was detected. The mixture was concentrated under vacuum to give a residue. The residue was purified by Prep-HPLC (Column: Welch Xtimate C18 150×25 mm×5 μm; Condition: water (10 mM NH₄HCO₃)-ACN, Begin 16, End 46, Gradient Time (min) 10, 100% B Hold Time (min) 2, FlowRate (mL/min):25) to give titled compound (0.27 g, 19.84% yield) as a white solid. LCMS: m/z=326.1 (M+H⁺).

### 1. Synthesis of tert-butyl (1-(4-chloropyridin-3-yl)-6-oxopiperidin-3-yl)(methyl)carbamate

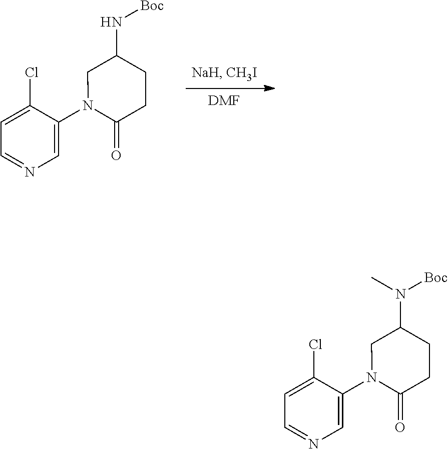

To a solution of tert-butyl (1-(4-chloropyridin-3-yl)-6-oxopiperidin-3-yl)carbamate (270 mg, 828.76 μmol, 1.0 eq.) in DMF (12.00 mL) was added NaH (49.72 mg, 1.24 mmol, 60% purity, 1.5 eq.) at 0° C. The reaction mixture was stirred for 10 minutes at 0° C. Then CH3I (15.94 g, 112.30 mmol, 135.51 eq.) was added into the reaction mixture. Removed the ice bath, the reaction mixture was stirred at 0° C. for 30 minutes. LCMS showed the reaction was completed, the reaction was quenched with HCl (0.2 mL, 1 M). The mixture was purified by Prep-TLC (DCM/MeOH=10/1) to give titled compound (190 mg, 67.47% yield) as yellow oil. LCMS: m/z=340.1 (M+H⁺).

### 2. Synthesis of tert-butyl (1-(4-(4-((2-(tert-butyl)-2H-tetrazole-5-carboxamido)methyl)-3-methylphenyl)pyridin-3-yl)-6-oxopiperidin-3-yl)(methyl)carbamate

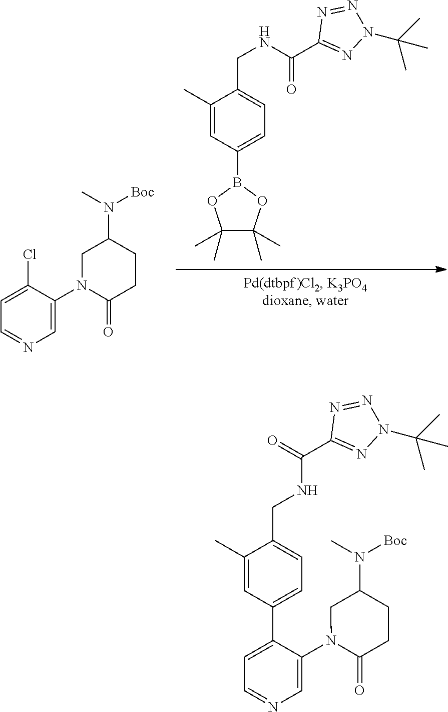 [SMILES: CN(C(=O)OC(C)(C)C)C1CCC(=O)N(c2cnccc2Cl)C1 ; Cc1cc(B2OC(C)(C)C(C)(C)O2)ccc1CNC(=O)c1nnn(C(C)(C)C)n1 ; Cc1cc(-c2ccncc2N2CC(N(C)C(=O)OC(C)(C)C)CCC2=O)ccc1CNC(=O)c1nnn(C(C)(C)C)n1]

To a solution of 2-(tert-butyl)-N-(2-methyl-4-(4,4,5,5-tetramethyl-1,3,2-dioxaborolan-2-yl)benzyl)-2H-tetrazole-5-carboxamide (223.26 mg, 559.13 μmol, 1.0 eq.) in dioxane (15.00 mL) and water (3.00 mL) was added tert-butyl (1-(4-chloropyridin-3-yl)-6-oxopiperidin-3-yl)(methyl)carbamate (190.00 mg, 559.13 μmol, 1.0 eq.), K₂CO₃ (231.82 mg, 1.68 mmol, 3.0 eq.) and Pd(dtbpf)Cl₂ (36.44 mg, 55.91 μmol, 0.1 eq.) at 15° C. The mixture was stirred at 90° C. under N₂ for 2 hours. LCMS showed the reaction was completed. The mixture was concentrated under vacuum to give crude product. The crude product was purified by Prep-HPLC (DCM/MeOH=10/1) to give titled compound (200.00 mg, 62.03% yield) as yellow oil. LCMS: m/z=577.4 (M+H⁺).

### 4. Synthesis of 2-(tert-butyl)-N-(2-methyl-4-(3-(5-(methylamino)-2-oxopiperidin-1-yl)pyridin-4-yl)benzyl)-2H-tetrazole-5-carboxamide

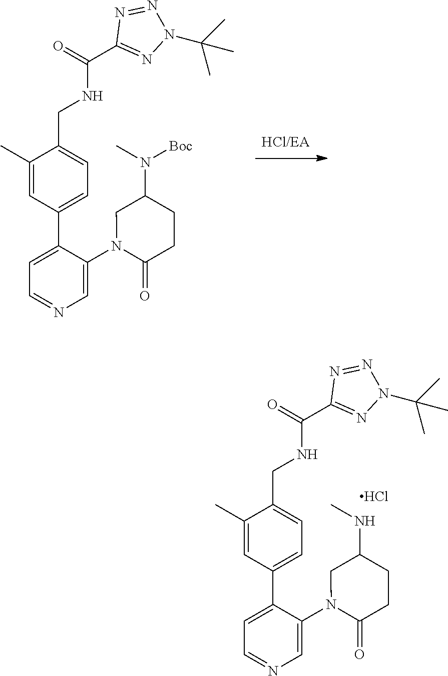 [SMILES: Cc1cc(-c2ccncc2N2CC(N(C)C(=O)OC(C)(C)C)CCC2=O)ccc1CNC(=O)c1nnn(C(C)(C)C)n1 ; CNC1CCC(=O)N(c2cnccc2-c2ccc(CNC(=O)c3nnn(C(C)(C)C)n3)c(C)c2)C1 ; Cl]

To a solution of tert-butyl (1-(4-(4-((2-(tert-butyl)-2H-tetrazole-5-carboxamido)methyl)-3-methylphenyl)pyridin-3-yl)-6-oxopiperidin-3-yl)(methyl)carbamate (200.00 mg, 346.81 μmol, 1.0 eq.) in DCM (15 mL) was added HCl/EA (8 mL, 4 M) at 15° C. The mixture was stirred at 15° C. for 1 hour. LCMS showed the reaction was completed. The mixture was concentrated under vacuum to give titled compound (150 mg, crude, hydrochloride) as a yellow solid. LCMS: m/z=477.3 (M+H⁺).

### 5. Synthesis of 2-(tert-butyl)-N-(2-methyl-4-(3-(5-(N-methylacrylamido)-2-oxopiperidin-1-yl)pyridin-4-yl)benzyl)-2H-tetrazole-5-carboxamide

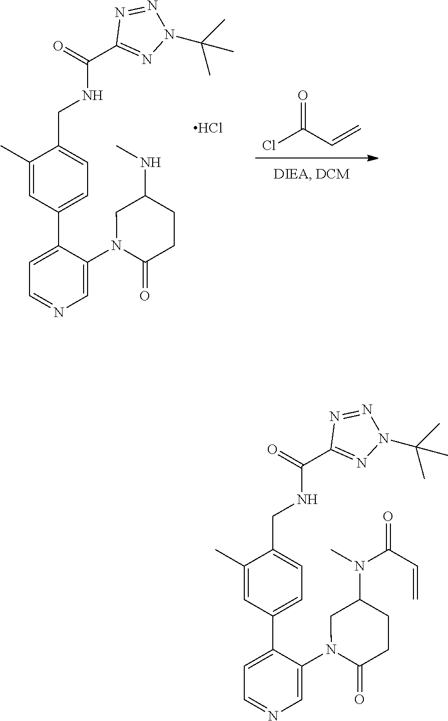

To a solution of 2-(tert-butyl)-N-(2-methyl-4-(3-(5-(methylamino)-2-oxopiperidin-1-yl)pyridin-4-yl)benzyl)-2H-tetrazole-5-carboxamide (150.00 mg, 292.38 μmol, hydrochloride, 1.0 eq.) in DCM (20 mL) was added DIPEA (75.57 mg, 584.76 μmol, 2.0 eq.) and acryloyl chloride (26.46 mg, 292.38 mmol, 1.0 eq.) at 15° C. The mixture was stirred at 15° C. for 10 minutes. LCMS showed the reaction was completed. The mixture was quenched with MeOH (1 mL) and concentrated under vacuum. The residue was purified by Prep-HPLC (Column: Welch Xtimate C18 150×25 mm×5 μm; Condition: water (10 mM NH₄HCO₃)-ACN, Begin B 23 End B 53, Gradient Time (min) 15, 100% B Hold Time (min) 2, Flow Rate (mL/min): 25) to give titled compound (100 mg, 64.46% yield) as a yellow solid. LCMS: m/z=531.3 (M+H⁺).

### 6. Synthesis of(S)-2-(tert-butyl)-N-(2-methyl-4-(3-(5-(N-methylacrylamido)-2-oxopiperidin-1-yl)pyridin-4-yl)benzyl)-2H-tetrazole-5-carboxamide and (R)-2-(tert-butyl)-N-(2-methyl-4-(3-(5-(N-methylacrylamido)-2-oxopiperidin-1-yl)pyridin-4-yl)benzyl)-2H-tetrazole-5-carboxamide

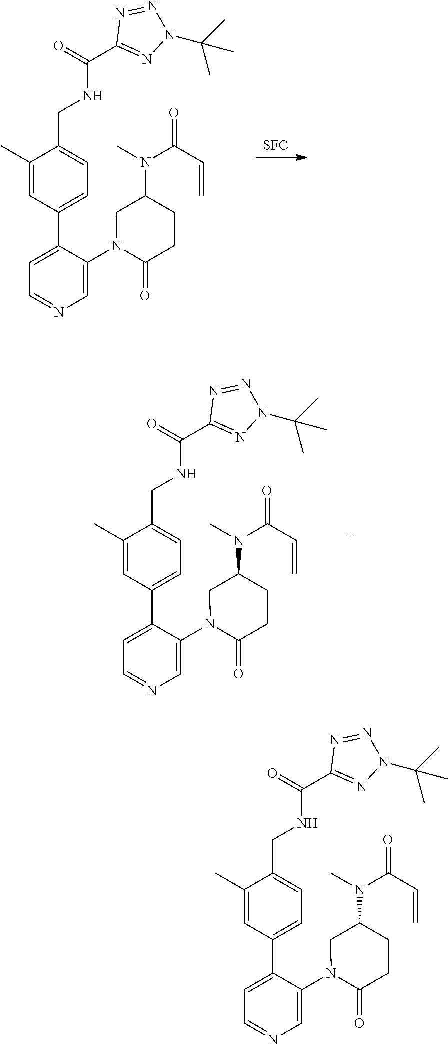 [SMILES: C=CC(=O)N(C)C1CCC(=O)N(c2cnccc2-c2ccc(CNC(=O)c3nnn(C(C)(C)C)n3)c(C)c2)C1 ; "SFC" ; C=CC(=O)N(C)[C@H]1CCC(=O)N(c2cnccc2-c2ccc(CNC(=O)c3nnn(C(C)(C)C)n3)c(C)c2)C1 ; C=CC(=O)N(C)[C@@H]1CCC(=O)N(c2cnccc2-c2ccc(CNC(=O)c3nnn(C(C)(C)C)n3)c(C)c2)C1]

The 2-(tert-butyl)-N-(2-methyl-4-(3-(5-(N-methylacrylamido)-2-oxopiperidin-1-yl)pyridin-4-yl)benzyl)-2H-tetrazole-5-carboxamide (100.00 mg, 188.46 μmol, 1.0 eq.) was purified by SFC (Column: DAICEL CHIRALCEL OD-H (250 mm×30 mm, 5 μm); Condition: 0.1% NH₃H₂O ETOH, Begin B 45, End B 45, Flow Rate (mL/min): 80) to give Peak 1 (32.00 mg, 32.00% yield, 100.00% purity, ee: 98.36%) as a white solid. LCMS: m/z=531.4 (M+H⁺). ¹H NMR (500 MHz, DMSO-d6) δ ppm=9.55 (s, 1H), 8.67-8.30 (m, 2H), 7.50-7.35 (m, 2H), 7.21 (s, 2H), 6.91-5.43 (m, 3H), 4.54 (s, 2H), 3.81-3.63 (m, 1H), 3.32-2.66 (m, 5H), 2.41 (s, 3H), 2.38-2.12 (m, 2H), 1.85-1.62 (m, 11H).

and Peak 2 (32.90 mg, 32.90% yield, 100.00% purity, ee: 97.28%) as a white solid. LCMS: m/z=531.4 (M+H⁺). ¹H NMR (500 MHz, DMSO-d6) δ ppm=9.56-9.50 (m, 1H), 8.62-8.42 (m, 2H), 7.50-7.36 (m, 2H), 7.21 (s, 2H), 6.87-5.46 (m, 3H), 4.54 (s, 2H), 3.80-3.62 (m, 1H), 3.32-2.67 (m, 5H), 2.41 (s, 3H), 2.38-2.11 (m, 2H), 1.85-1.68 (m, 11H).

### Example 54: tert-butyl 3-[4-[4-[[(1-tert-butyltriazole-4-carbonyl)amino]methyl]-3-methyl-phenyl]-3-pyridyl]-3,6-diazabicyclo[3.2.1]octane-6-carboxylate

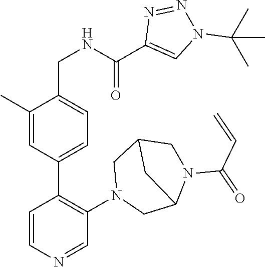 [SMILES: C=CC(=O)N1CC2CC1CN(c1cnccc1-c1ccc(CNC(=O)c3cn(C(C)(C)C)nn3)c(C)c1)C2]

### 1. Synthesis of tert-butyl 3-(4-chloro-3-pyridyl)-3,6-diazabicyclo[3.2.1]octane-6-carboxylate

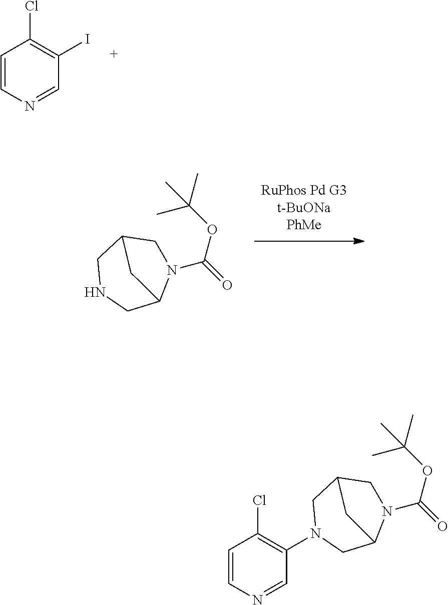 [SMILES: Clc1ccncc1I ; CC(C)(C)OC(=O)N1CC2CNCC1C2 ; CC(C)(C)OC(=O)N1CC2CC1CN(c1cnccc1Cl)C2]

4-chloro-3-iodo-pyridine (563.95 mg, 2.36 mmol), tert-butyl 3,6-diazabicyclo[3.2.1]octane-6-carboxylate (500.00 mg, 2.36 mmol), RuPhos Pd G3 (196.99 mg, 235.53 umol) and Sodium tert-butoxide (679.02 mg, 7.07 mmol) were dissolved in Toluene (10.00 mL). The reaction was bubbled with nitrogen for 5 minutes and stirred at 100° C. for 16 hours. The cooled mix was diluted with EtOAc and filtrated through celite, the concentrated residue was chromatographed on Si gel (HE/EA 0-100%) to give tert-butyl 3-(4-chloro-3-pyridyl)-3,6-diazabicyclo[3.2.1]octane-6-carboxylate (443.00 mg, 1.37 mmol, 57.97% yield). LCMS: Rt=0.83 min, m/z 324.1 (M+H⁺). ¹H NMR (400 MHz, CDCl₃) δ 8.32 (d, J=5.27 Hz, 1H), 8.17 (t, J=5.52 Hz, 1H), 7.26 (d, J=5.02 Hz, 1H), 4.20 (t, J=4.77 Hz, 0.5H), 4.07-4.11 (m, 0.5H), 3.76 (dd, J=4.27, 11.04 Hz, 1H), 3.55-3.69 (m, 1H), 3.36-3.48 (m, 1H), 3.27-3.36 (m, 1H), 3.15 (br d, J=10.29 Hz, 1H), 2.80 (t, J=11.29 Hz, 1H), 2.51 (br s, 1H), 1.95-2.04 (m, 1H), 1.72 (dd, J=5.02, 11.04 Hz, 1H), 1.47 (d, J=7.53 Hz, 9H).

### 2. Synthesis of benzyl N-[[2-methyl-4-(4,4,5,5-tetramethyl-1,3,2-dioxaborolan-2-yl)phenyl]methyl]carbamate

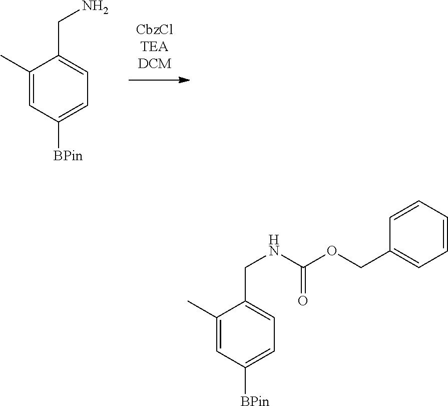

[2-methyl-4-(4,4,5,5-tetramethyl-1,3,2-dioxaborolan-2-yl)phenyl]methanamine (1.18 g, 4.16 mmol, Hydrochloride) in DCM (10.00 mL) was cooled to 0° C. and Cbz chloride (709.67 mg, 4.16 mmol, 591.39 uL) was added followed by dropwise addition of TEA (1.26 g, 12.48 mmol, 1.73 mL). After 1 h, the mix was filtrated through celite and the concentrated residue was chromatographed on Si gel (HE/EA 0-50%) to give benzyl N-[[2-methyl-4-(4,4,5,5-tetramethyl-1,3,2-dioxaborolan-2-yl)phenyl]methyl]carbamate (1.04 g, 2.73 mmol, 65.57% yield). LCMS: Rt=1.01 min, m/z 382.3 (M+H⁺).

### 3. Synthesis of tert-butyl 3-(4-(4-((((benzyloxy)carbonyl)amino)methyl)-3-methylphenyl)pyridin-3-yl)-3,6-diazabicyclo[3.2.1]octane-6-carboxylate

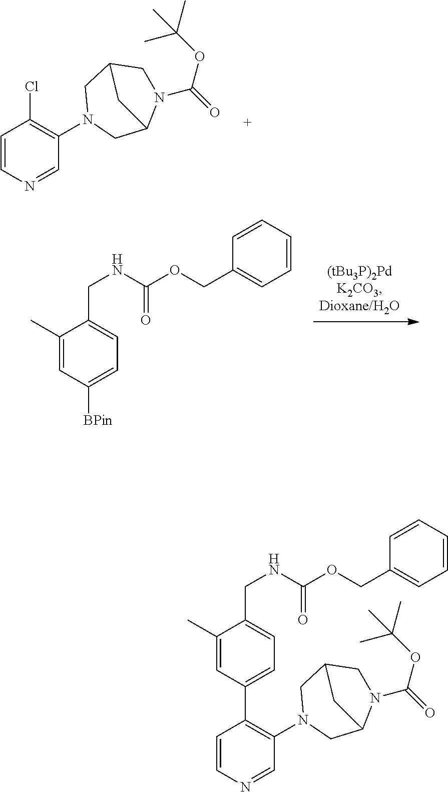

tert-butyl 3-(4-chloropyridin-3-yl)-3,6-diazabicyclo[3.2.1]octane-6-carboxylate (351.00 mg, 953.11 umol), benzyl N-[[2-methyl-4-(4,4,5,5-tetramethyl-1,3,2-dioxaborolan-2-yl)phenyl]methyl]carbamate (545.09 mg, 1.43 mmol), Potassium carbonate (526.90 mg, 3.81 mmol) and palladium; tritert-butylphosphane (48.71 mg, 95.31 umol) in Dioxane (4.20 mL) and water (700.21 uL) was degassed and heated to 100° C. for 16h. The cooled mix was filtrated through celite and concentrated residue was chromatographed on Si gel (HE/EA 0-100%) to give tert-butyl 3-(4-(4-((((benzyloxy)carbonyl)amino)methyl)-3-methylphenyl)pyridin-3-yl)-3,6-diazabicyclo[3.2.1]octane-6-carboxylate (254.00 mg, 468.06 umol, 49.11% yield) as a white solid. LCMS: Rt=0.89 min, m/z 543.3 (M+H⁺).

### 4. Synthesis of tert-butyl 3-[4-[4-(aminomethyl)-3-methyl-phenyl]-3-pyridyl]-3,6-diazabicyclo[3.2.1]octane-6-carboxylate

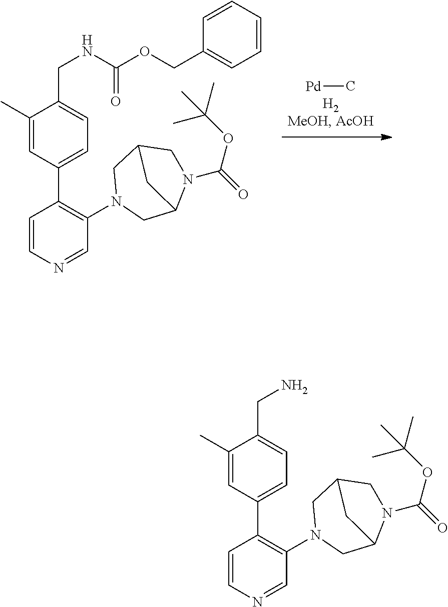

tert-butyl 3-[4-[4-(benzyloxycarbonylaminomethyl)-3-methyl-phenyl]-3-pyridyl]-3,6-diazabicyclo[3.2.1]octane-6-carboxylate (254.00 mg, 468.06 umol), Pd/C (99.62 mg, 46.81 umol, 5% purity) in MeOH (5.00 mL) was added Acetic acid (56.21 mg, 936.12 umol, 53.53 uL) and stirred under 1 atm of H₂ for 16h. The concentrated residue was diluted with DCM, washed with sat. aq. NaHCO₃, dried over Na₂SO₄ and chromatographed on Si gel (DCM/MeOH 0-30%) to give tert-butyl 3-[4-[4-(aminomethyl)-3-methyl-phenyl]-3-pyridyl]-3,6-diazabicyclo[3.2.1]octane-6-carboxylate (230.00 mg, 562.98 umol, 120.28% yield) as a yellow gel. LCMS: Rt=0.70 min, m/z 409.3 (M+H⁺).

### 5. Synthesis of tert-butyl 3-[4-[4-[[(1-tert-butyltriazole-4-carbonyl)amino]methyl]-3-methyl-phenyl]-3-pyridyl]-3,6-diazabicyclo[3.2.1]octane-6-carboxylate

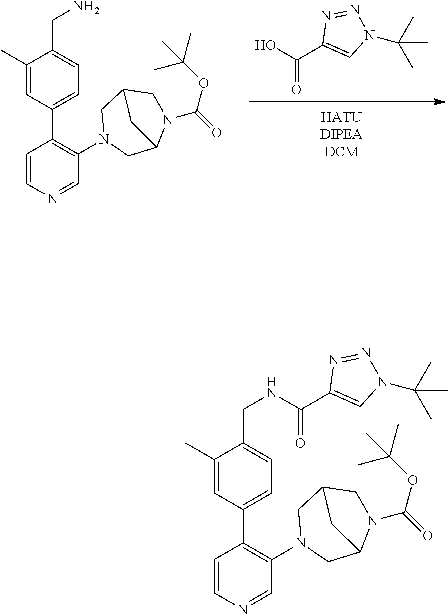 [SMILES: Cc1cc(-c2ccncc2N2CC3CC(C2)N(C(=O)OC(C)(C)C)C3)ccc1CN ; CC(C)(C)n1cc(C(=O)O)nn1 ; Cc1cc(-c2ccncc2N2CC3CC(C2)N(C(=O)OC(C)(C)C)C3)ccc1CNC(=O)c1cn(C(C)(C)C)nn1]

tert-butyl 3-[4-[4-(aminomethyl)-3-methyl-phenyl]-3-pyridyl]-3,6-diazabicyclo[3.2.1]octane-6-carboxylate (80.00 mg, 195.82 umol), 1-tert-butyltriazole-4-carboxylic acid (33.13 mg, 195.82 umol) in DCM (2.00 mL) was added DIPEA (25.31 mg, 195.82 umol, 34.20 uL) and HATU (74.65 mg, 195.82 umol) and stirred at rt for 16h. The crude was chromatographed on Si gel (HE/EA 0-100%) to give tert-butyl 3-[4-[4-[[(1-tert-butyltriazole-4-carbonyl)amino]methyl]-3-methyl-phenyl]-3-pyridyl]-3,6-diazabicyclo[3.2.1]octane-6-carboxylate (43.90 mg, 78.43 umol, 40.05% yield) as a gel. LCMS: Rt=0.88 min, m/z 560.4 (M+H⁺).

### 6. Synthesis of 1-tert-butyl-N-[[4-[3-(3,6-diazabicyclo[3.2.1]octan-3-yl)-4-pyridyl]-2-methyl-phenyl]methyl]triazole-4-carboxamide

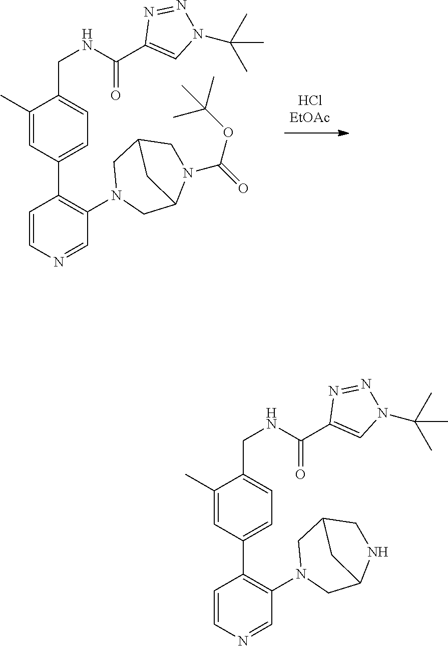 [SMILES: Cc1cc(-c2ccncc2N2CC3CC(C2)N(C(=O)OC(C)(C)C)C3)ccc1CNC(=O)c1cn(C(C)(C)C)nn1 ; Cc1cc(-c2ccncc2N2CC3CNC(C3)C2)ccc1CNC(=O)c1cn(C(C)(C)C)nn1]

tert-butyl 3-[4-[4-[[(1-tert-butyltriazole-4-carbonyl)amino]methyl]-3-methyl-phenyl]-3-pyridyl]-3,6-diazabicyclo[3.2.1]octane-6-carboxylate (43.90 mg, 78.43 umol) in EtOAc (2.00 mL) was added HCl (4 M, 78.43 uL) and stirred at rt for 16h. The crude was concentrated to give 1-tert-butyl-N-[[4-[3-(3,6-diazabicyclo[3.2.1]octan-3-yl)-4-pyridyl]-2-methyl-phenyl]methyl]triazole-4-carboxamide (38.40 mg, 77.41 umol, 98.70% yield, Hydrochloride) as a white solid and used as is for the next step. LCMS: Rt=0.63 min, m/z 460.3 (M+H⁺).

### 7. Synthesis of 1-tert-butyl-N-[[2-methyl-4-[3-(6-prop-2-enoyl-3,6-diazabicyclo[3.2.1]octan-3-yl)-4-pyridyl]phenyl]methyl]triazole-4-carboxamide

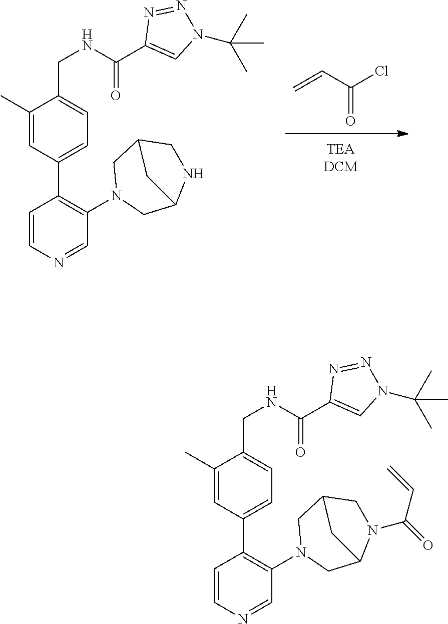

1-tert-butyl-N-[[4-[3-(3,6-diazabicyclo[3.2.1]octan-3-yl)-4-pyridyl]-2-methyl-phenyl]methyl]triazole-4-carboxamide (38.40 mg, 77.41 umol, Hydrochloride) in DCM (4.00 mL) was added TEA (15.67 mg, 154.82 umol, 21.47 uL) and stirred for 5 min. After cooling to 0° C., prop-2-enoyl chloride (8.41 mg, 92.89 umol, 7.58 uL) was added and stirred for 5 min and quenched with sat. aq. NaHCO₃ (10 mL). The organic layer was passed over an anh. Na₂SO₄ pad and concentrated. The residue was chromatographed on Si gel (EtOAc/MeOH 0-15%) to give 1-tert-butyl-N-[[2-methyl-4-[3-(6-prop-2-enoyl-3,6-diazabicyclo[3.2.1]octan-3-yl)-4-pyridyl]phenyl]methyl]triazole-4-carboxamide (7.00 mg, 12.95 umol, 16.73% yield, 95% purity). LCMS: Rt=0.56 min, m/z 514.3 (M+H⁺). ¹H NMR (400 MHz, CDCl₃) δ ppm 8.41 (m, 1H), 8.31 (m, 1H), 8.22 (m, 1H), 7.46-7.73 (m, 1H), 7.28-7.40 (m, 1H), 7.23-7.26 (m, 1H), 7.17-7.22 (m, 1H), 7.07 (m, 1H), 6.43-6.47 (m, 1H), 5.99-6.36 (m, 1H), 5.60-5.70 (m, 1H), 4.57-4.80 (m, 2H), 4.54 (t, J=4.39 Hz, 0.5 H), 4.02-4.06 (m, 0.5 H), 2.52-3.57 (m, 7H), 2.40-2.42 (m, 3H), 1.81-1.99 (m, 1H), 1.70-1.72 (m, 9H), 1.60-1.69 (m, 1H).

### Example 55: 5-tert-butyl-N-[[2-methyl-4-[3-(6-prop-2-enoyl-3,6-diazabicyclo[3.2.1]octan-3-yl)-4-pyridyl]phenyl]methyl]isoxazole-3-carboxamide

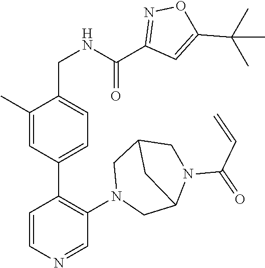 [SMILES: C=CC(=O)N1CC2CC1CN(c1cnccc1-c1ccc(CNC(=O)c3cc(C(C)(C)C)on3)c(C)c1)C2]

### 1. Synthesis of tert-butyl 3-[4-[4-[[(5-tert-butylisoxazole-3-carbonyl)amino]methyl]-3-methyl-phenyl]-3-pyridyl]-3,6-diazabicyclo[3.2.1]octane-6-carboxylate

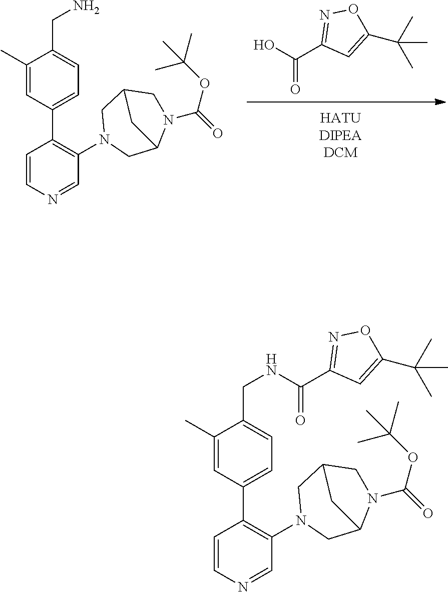 [SMILES: Cc1cc(-c2ccncc2N2CC3CC(C2)N(C(=O)OC(C)(C)C)C3)ccc1CN ; CC(C)(C)c1cc(C(=O)O)no1 ; Cc1cc(-c2ccncc2N2CC3CC(C2)N(C(=O)OC(C)(C)C)C3)ccc1CNC(=O)c1cc(C(C)(C)C)on1]

tert-butyl 3-[4-[4-(aminomethyl)-3-methyl-phenyl]-3-pyridyl]-3,6-diazabicyclo[3.2.1]octane-6-carboxylate (80.00 mg, 195.82 umol), 5-tert-butylisoxazole-3-carboxylic acid (33.13 mg, 195.82 umol) in DCM (2.00 mL) was added DIPEA (75.92 mg, 587.46 umol, 102.59 uL) and HATU (149.31 mg, 391.64 umol) and stirred at rt for 16h. The crude was chromatographed on si gel (HE/EA 0-100%) to give tert-butyl 3-[4-[4-[[(5-tert-butylisoxazole-3-carbonyl)amino]methyl]-3-methyl-phenyl]-3-pyridyl]-3,6-diazabicyclo[3.2.1]octane-6-carboxylate (18.40 mg, 31.23 umol, 15.95% yield, 95% purity). LCMS: Rt=1.01 min, m/z 560.4 (M+H⁺).

### 2. 5-tert-butyl-N-[[4-[3-(3,6-diazabicyclo[3.2.1]octan-3-yl)-4-pyridyl]-2-methyl-phenyl]methyl]isoxazole-3-carboxamide

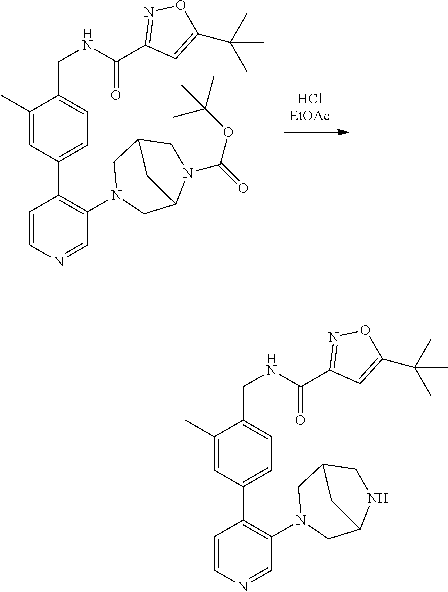 [SMILES: Cc1cc(-c2ccncc2N2CC3CC(C2)N(C(=O)OC(C)(C)C)C3)ccc1CNC(=O)c1cc(C(C)(C)C)on1 ; Cc1cc(-c2ccncc2N2CC3CNC(C3)C2)ccc1CNC(=O)c1cc(C(C)(C)C)on1]

tert-butyl 3-[4-[4-[[(5-tert-butylisoxazole-3-carbonyl)amino]methyl]-3-methyl-phenyl]-3-pyridyl]-3,6-diazabicyclo[3.2.1]octane-6-carboxylate (18.40 mg, 32.87 umol) in EtOAc (2.00 mL) was added HCl (4 M, 49.30 uL) and stirred at rt for 16h. The crude was concentrated to give 5-tert-butyl-N-[[4-[3-(3,6-diazabicyclo[3.2.1]octan-3-yl)-4-pyridyl]-2-methyl-phenyl]methyl]isoxazole-3-carboxamide (17.00 mg, 34.27 umol, 104.26% yield, Hydrochloride) as a white solid and used as is for the next step. LCMS: Rt=0.74 min, m/z 460.3 (M+H⁺).

### 3. 5-tert-butyl-N-[[2-methyl-4-[3-(6-prop-2-enoyl-3, 6-diazabicyclo[3.2.1]octan-3-yl)-4-pyridyl]phenyl]methyl]isoxazole-3-carboxamide

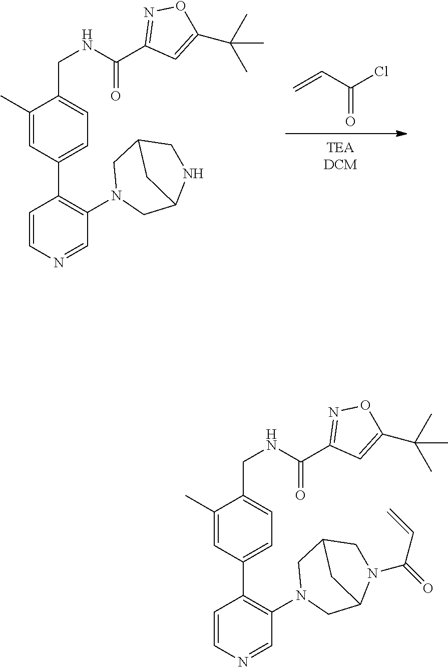

5-tert-butyl-N-[[4-[3-(3,6-diazabicyclo[3.2.1]octan-3-yl)-4-pyridyl]-2-methyl-phenyl]methyl]isoxazole-3-carboxamide (17.00 mg, 34.27 umol, Hydrochloride) in DCM (3.00 mL) was added TEA (6.94 mg, 68.54 umol, 9.51 uL) and stirred for 5 min. After cooling to 0° C., prop-2-enoyl chloride (3.72 mg, 41.12 umol, 3.35 uL) was added and stirred for 5 min and quenched with sat aq NaHCO₃ (10 mL). The organic layer was passed over an anh. Na₂SO₄ pad and concentrated. The residue was chromatographed on Si gel (EtOAc/MeOH 0-15%) to give 5-tert-butyl-N-[[2-methyl-4-[3-(6-prop-2-enoyl-3,6-diazabicyclo[3.2.1]octan-3-yl)-4-pyridyl]phenyl]methyl]isoxazole-3-carboxamide (5.20 mg, 9.62 umol, 28.06% yield, 95% purity). LCMS: Rt=0.65 min, m/z 514.3 (M+H⁺). ¹H NMR (400 MHz, CDCl₃) δ ppm 8.38-8.48 (m, 1H), 8.28-8.36 (m, 1H), 7.48-7.81 (m, 1H), 7.29-7.37 (m, 1H), 7.19-7.22 (m, 1H), 7.15-7.18 (m, 1H), 7.03-7.12 (m, 1H), 6.48-6.49 (m, 1H), 5.91-6.32 (m, 1H), 5.52-5.78 (m, 1H), 3.96-4.92 (m, 4H), 2.94-3.48 (m, 5H), 2.64-2.75 (m, 1H), 2.47-2.56 (m, 1H), 2.34-2.41 (m, 3H), 1.79-1.98 (m, 1H), 1.60-1.75 (m, 1H), 1.33-1.42 (m, 9H).

### Example 56: 1-tert-butyl-N-[[4-[3-fluoro-5-[(3R)-3-[methyl(prop-2-enoyl)amino]-1-piperidyl]-4-pyridyl]-2-methyl-phenyl]methyl]triazole-4-carboxamide

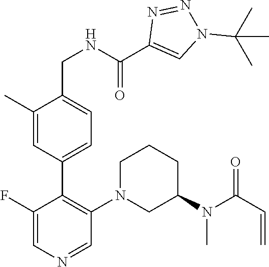 [SMILES: C=CC(=O)N(C)[C@@H]1CCCN(c2cncc(F)c2-c2ccc(CNC(=O)c3cn(C(C)(C)C)nn3)c(C)c2)C1]

### 1. Synthesis of tert-butyl N-[(3R)-1-(4-chloro-5-fluoro-3-pyridyl)-3-piperidyl]-N-methyl-carbamate

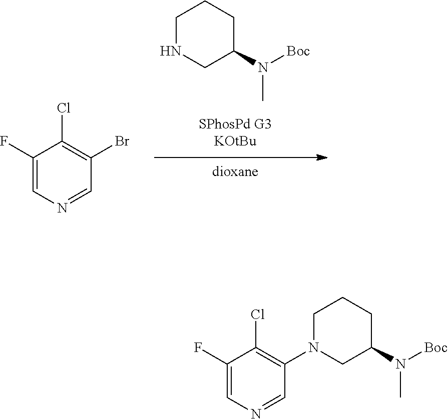 [SMILES: CN(C(=O)OC(C)(C)C)[C@@H]1CCCNC1 ; Fc1cncc(Br)c1Cl ; CN(C(=O)OC(C)(C)C)[C@@H]1CCCN(c2cncc(F)c2Cl)C1]

A flask was charged with 3-bromo-4-chloro-5-fluoro-pyridine (50.00 mg, 237.61 umol), tert-butyl N-methyl-N-[(3R)-3-piperidyl]carbamate (48.37 mg, 225.73 umol), potassium tert-butoxide (53.32 mg, 475.22 umol), SPhosPd G3 (18.54 mg, 23.76 umol). The flask was purged and refilled with N₂ 3 times, followed by the addition of degassed dioxane (2.00 mL). The resulting mixture was heated to reflux until all starting material was consumed after 1 h monitored by LCMS. The concentrated crude was purified by column chromatography (12 g, SiO₂, 0-35% EtOAc/EtOH 3:1 in heptane) to give tert-butyl N-[(3R)-1-(4-chloro-5-fluoro-3-pyridyl)-3-piperidyl]-N-methyl-carbamate (38.00 mg, 110.52 umol, 46.51% yield). LCMS: m/z 344.2 (M+H⁺).

### 2. Synthesis of tert-butyl N-[(3R)—]-[4-[4-[[(1-tert-butyltriazole-4-carbonyl)amino]methyl]-3-methyl-phenyl]-5-fluoro-3-pyridyl]-3-piperidyl]-N-methyl-carbamate

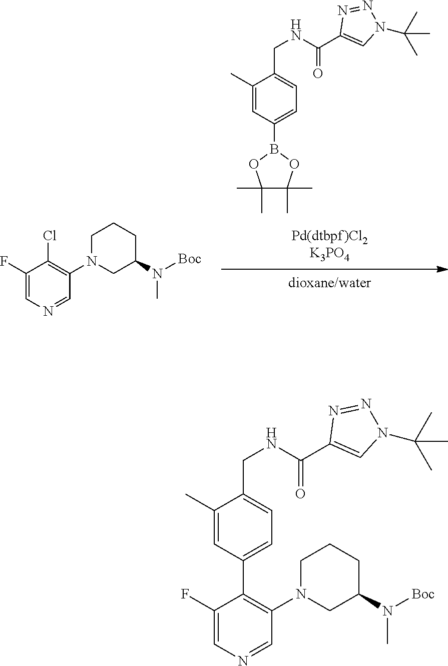 [SMILES: Cc1cc(B2OC(C)(C)C(C)(C)O2)ccc1CNC(=O)c1cn(C(C)(C)C)nn1 ; CN(C(=O)OC(C)(C)C)[C@@H]1CCCN(c2cncc(F)c2Cl)C1 ; Cc1cc(-c2c(F)cncc2N2CCC[C@@H](N(C)C(=O)OC(C)(C)C)C2)ccc1CNC(=O)c1cn(C(C)(C)C)nn1]

A flask was charged with tert-butyl N-[(3R)-1-(4-chloro-5-fluoro-3-pyridyl)-3-piperidyl]-N-methyl-carbamate (38.00 mg, 110.52 umol), 1-tert-butyl-N-[[2-methyl-4-(4,4,5,5-tetramethyl-1,3,2-dioxaborolan-2-yl)phenyl]methyl]triazole-4-carboxamide (48.42 mg, 121.57 umol), Potassium phosphate tribasic (46.92 mg, 221.04 umol), Pd(dtbpf)Cl₂ (10.80 mg, 16.58 umol). The flask was purged and refilled with N₂ 3 times, followed by the addition of previously degassed water (100.00 uL) in dioxane (1.90 mL). The mixture was refluxed for overnight and brought to rt, diluted with EtOAc, washed with water, and the org phase was separated and concentrated under reduced pressure. The residue was purified by column chromatography (12 g, SiO₂, 0-40% EtOAc/EtOH 3:1 in heptane) to give tert-butyl N-[(3R)-1-[4-[4-[[(1-tert-butyltriazole-4-carbonyl)amino]methyl]-3-methyl-phenyl]-5-fluoro-3-pyridyl]-3-piperidyl]-N-methyl-carbamate (31.50 mg, 54.34 umol, 49.17% yield) as a pale yellow oil. LCMS: m/z 580.4 (M+H⁺).

### 3. Synthesis of 1-tert-butyl-N-[[4-[3-fluoro-5-[(3R)-3-(methylamino)-1-piperidyl]-4-pyridyl]-2-methyl-phenyl]methyl]triazole-4-carboxamide

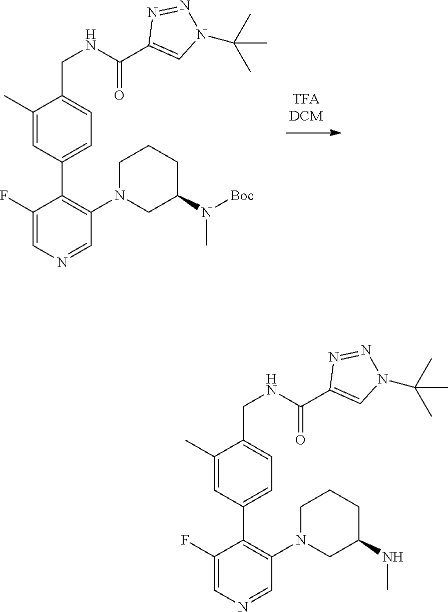 [SMILES: Cc1cc(-c2c(F)cncc2N2CCC[C@@H](N(C)C(=O)OC(C)(C)C)C2)ccc1CNC(=O)c1cn(C(C)(C)C)nn1 ; CN[C@@H]1CCCN(c2cncc(F)c2-c2ccc(CNC(=O)c3cn(C(C)(C)C)nn3)c(C)c2)C1]

To a solution of tert-butyl N-[(3R)-1-[4-[4-[[(1-tert-butyltriazole-4-carbonyl)amino]methyl]-3-methyl-phenyl]-5-fluoro-3-pyridyl]-3-piperidyl]-N-methyl-carbamate (31.50 mg, 54.34 umol) in DCM (2.00 mL) was added TFA (745.00 mg, 6.53 mmol, 500.00 uL) at 0° C. The resulting reaction mixture was stirred at that temperature for 30 min until starting material consumed. The volatiles were removed under reduced pressure and the resulting brown residue was purified by column chromatography (4 g SiO₂, 0-15% MeOH (5% NH40H) in DCM) yielding the titled compound 1-tert-butyl-N-[[4-[3-fluoro-5-[(3R)-3-(methylamino)-1-piperidyl]-4-pyridyl]-2-methyl-phenyl]methyl]triazole-4-carboxamide (25.00 mg, 52.13 umol, 95.93% yield) as a pale yellow oil. LCMS: m/z 480.3 (M+H⁺).

### 4. Synthesis of 1-tert-butyl-N-[[4-[3-fluoro-5-[(3R)-3-[methyl(prop-2-enoyl)amino]-1-piperidyl]-4-pyridyl]-2-methyl-phenyl]methyl]triazole-4-carboxamide

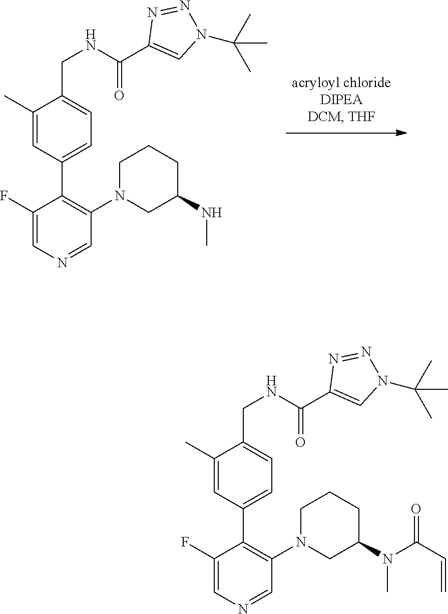 [SMILES: CN[C@@H]1CCCN(c2cncc(F)c2-c2ccc(CNC(=O)c3cn(C(C)(C)C)nn3)c(C)c2)C1 ; C=CC(=O)N(C)[C@@H]1CCCN(c2cncc(F)c2-c2ccc(CNC(=O)c3cn(C(C)(C)C)nn3)c(C)c2)C1]

To a solution of 1-tert-butyl-N-[[4-[3-fluoro-5-[(3R)-3-(methylamino)-1-piperidyl]-4-pyridyl]-2-methyl-phenyl]methyl]triazole-4-carboxamide (25.00 mg, 52.13 umol) and DIPEA (20.21 mg, 156.39 umol, 27.31 uL) in DCM (2.00 mL) and THF (500.00 uL) was added a solution of Acryloyl chloride (5.90 mg, 65.16 umol, 5.32 uL) in THF (100.10 uL) at 0° C. After LCMS indicated full conversion of the starting material (30 min), the reaction mixture was diluted with DCM (5 mL) transferred to a separation funnel and washed with sat. aq. NH₄Cl, water and brine. The organic phase was dried over Na₂SO₄, filtered and concentrated. The concentrated residue was purified with column chromatography (12 g SiO₂, 0-15% MeOH (5% NH₄OH) in DCM) to give the title compound 1-tert-butyl-N-[[4-[3-fluoro-5-[(3R)-3-[methyl(prop-2-enoyl)amino]-1-piperidyl]-4-pyridyl]-2-methyl-phenyl]methyl]triazole-4-carboxamide (13.00 mg, 24.36 umol, 46.73% yield) as an off white solid. LCMS: Rt=1.56 min, m/z=534.3 (M+H⁺). ¹H NMR (500 MHz, MeOH-d₄) □ 8.48 (s, 1H), 8.19 (br s, 1H), 8.14 (br d, J=7.33 Hz, 1H), 7.41-7.51 (m, 1H), 7.30-7.41 (m, 2H), 6.02-6.71 (m, 2H), 5.69 (td, J=3.36, 9.16 Hz, 1H), 4.86 (s, 2H), 4.58-4.75 (m, 2H), 3.59-4.36 (m, 1H), 3.02-3.22 (m, 2H), 2.50-2.96 (m, 5H), 2.44 (s, 3H), 1.73-1.85 (m, 1H), 1.70 (s, 9H), 1.40-1.69 (m, 2H).

### Example 57: 5-(tert-butyl)-N-(4-(3-(4-(cycobut-1-ene-1-carbonyl)piperazin-1-yl)pyridin-4-yl)-2-methylbenzyl)-1,2,4-oxadiazole-3-carboxamide

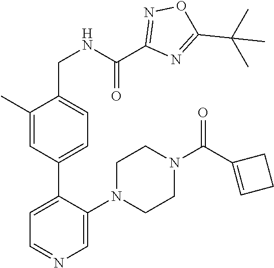 [SMILES: Cc1cc(-c2ccncc2N2CCN(C(=O)C3=CCC3)CC2)ccc1CNC(=O)c1noc(C(C)(C)C)n1]

### 1. Synthesis of tert-butyl 4-(4-(4-((5-(tert-butyl)-1,2,4-oxadiazole-3-carboxamido)methyl)-3-methylphenyl)pyridin-3-yl)piperazine-1-carboxylate

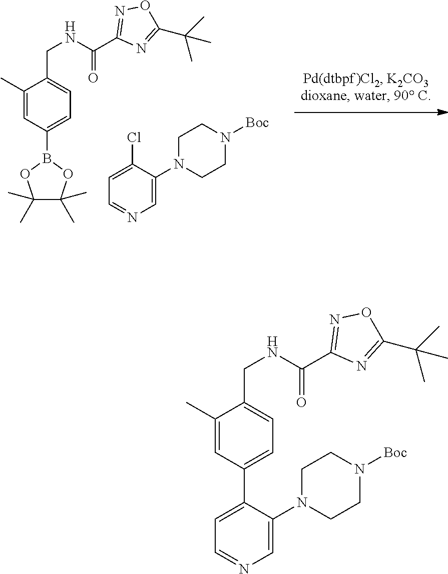

To a solution of tert-butyl 4-(4-chloro-3-pyridyl)piperazine-1-carboxylate (5 g, 16.8 mmol) in dioxane (120 mL) and water (20 mL) was added potassium carbonate (6.96 g, 50.37 mmol) and 5-tert-butyl-N-[[2-methyl-4-(4,4,5,5-tetramethyl-1,3,2-dioxaborolan-2-yl)phenyl]methyl]-1,2,4-oxadiazole-3-carboxamide (8.05 g, 20.2 mmol) at 20° C. Then [1,1′-bis(di-tert-butylphosphino)ferrocene]dichloropalladium(II) (2.19 g, 3.36 mmol) was added to the mixture at 20° C. The reaction was stirred at 90° C. under N₂ for 12 hours. The mixture was concentrated under vacuum and was purified by chromatography column on silica gel (petroleum ether/ethyl acetate=1/0 to 1/1) to give tert-butyl 4-(4-(4-((5-(tert-butyl)-1,2,4-oxadiazole-3-carboxamido)methyl)-3-methylphenyl)pyridin-3-yl)piperazine-1-carboxylate as yellow oil (6 g, 63% yield). LCMS: m/z=535.5 (M+H⁺).

### 2. Synthesis of 5-(tert-butyl)-N-(2-methyl-4-(3-(piperazin-1-yl)pyridin-4-yl)benzyl)-1,2,4-oxadiazole-3-carboxamide hydrochloride

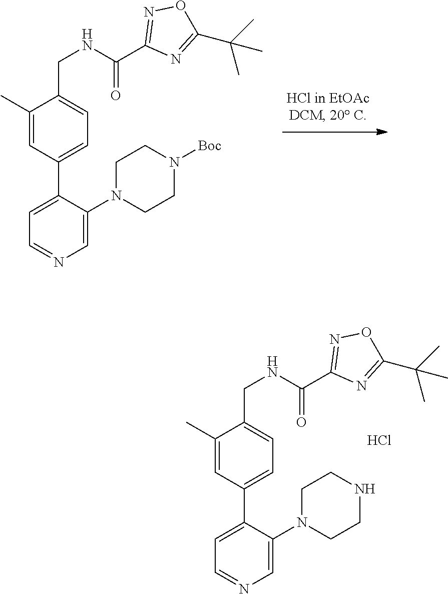 [SMILES: Cc1cc(-c2ccncc2N2CCN(C(=O)OC(C)(C)C)CC2)ccc1CNC(=O)c1noc(C(C)(C)C)n1 ; Cc1cc(-c2ccncc2N2CCNCC2)ccc1CNC(=O)c1noc(C(C)(C)C)n1 ; Cl]

To a solution of tert-butyl 4-[4-[4-[[(5-tert-butyl-1,2,4-oxadiazole-3-carbonyl)amino]methyl]-3-methyl-phenyl]-3-pyridyl]piperazine-1-carboxylate (2 g, 3.74 mmol) in DCM (200 mL) was added HCl solution in ethyl acetate (20 mL) at 20° C. The mixture was stirred at 20° C. for 30 mins. The mixture was concentrated under vacuum to give the crude, which was purified by prep-HPLC (Column: Boston Uni C18 40*150*5 μm; Condition: water (0.05% HCl)-ACN, Begin B 0, End B 30; Gradient Time (min): 10; 100% B Hold Time (min): 2; Flow Rate (ml/min): 60) to give 5-tert-butyl-N-[[2-methyl-4-(3-piperazin-1-yl-4-pyridyl)phenyl]methyl]-1,2,4-oxadiazole-3-carboxamide hydrochloride as a yellow solid (1.11 g, yield 63% yield). LCMS: m/z=435.2 (M+H⁺).

### 3. Synthesis of 5-(tert-butyl)-N-(4-(3-(4-(cyclobut-1-ene-1-carbonyl)piperazin-1-yl)pyridin-4-yl)-2-methylbenzyl)-1,2,4-oxadiazole-3-carboxamide

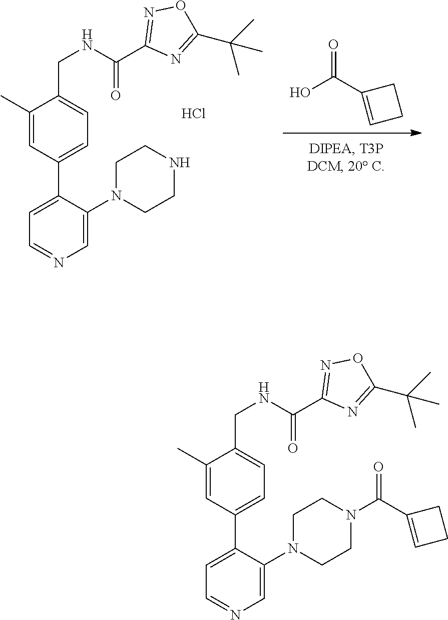 [SMILES: Cc1cc(-c2ccncc2N2CCNCC2)ccc1CNC(=O)c1noc(C(C)(C)C)n1 ; Cl ; O=C(O)C1=CCC1 ; Cc1cc(-c2ccncc2N2CCN(C(=O)C3=CCC3)CC2)ccc1CNC(=O)c1noc(C(C)(C)C)n1]

A vial was charged with 5-tert-butyl-N-[[2-methyl-4-(3-piperazin-1-yl-4-pyridyl)phenyl]methyl]-1,2,4-oxadiazole-3-carboxamide hydrochloride (55 mg, 117 μmol), cyclobutene-1-carboxylic acid (23 mg, 234 μmol) and DCM (1 mL). Then, DIPEA (60 mg, 467 μmol, 81 μL) was added, followed by T3P (186 mg, 292 μmol, 50% purity). After stirring at room temperature overnight, the mixture was diluted with water and extracted with ethyl acetate (×2). The combined organic extracts were washed with brine, dried with Na₂SO₄, filtered, and the filtrate was concentrated. The residual material was purified on acidic prep-HPLC (water (0.1% TFA)-ACN, Begin B 10, End B 95) to give a white solid after lyophilization. This material contains a small impurity and was re-purified on basic prep-HPLC ((water (0.1% NH₄OH)-ACN, Begin B 10, End B 90) to give 5-tert-butyl-N-[[4-[3-[4-(cyclobutene-1-carbonyl)piperazin-1-yl]-4-pyridyl]-2-methyl-phenyl]methyl]-1,2,4-oxadiazole-3-carboxamide as a white solid (3.3 mg, 5% yield). LCMS: m/z=515.5 (M+H⁺). ¹H NMR (DMSO-d₆, 400 MHz): δ=9.48 (dt, J=11.9, 6.1 Hz, 1H), 8.70 (m, 0.5H), 8.52 (m, 0.5H), 8.39-8.46 (m, 1H), 7.93 (d, J=6.3 Hz, 0.5H), 7.64-7.78 (m, 2.5H), 7.36-7.47 (m, 1H), 6.40-6.49 (m, 1H), 4.47-4.56 (m, 2H), 3.45-3.65 (br s, 2H), 3.02-3.15 (m, 1H), 2.93 (br d, J=4.0 Hz, 4H), 2.65-2.72 (m, 2H), 2.35-2.45 (m, 5H), 2.18-2.26 (m, 1H), 1.43 (s, 9H).

### Example 58: (E)-5-(tert-butyl)-N-(2-methyl-4-(3-(4-(4,4,4-trifluorobut-2-enoyl)piperazin-1-yl)pyridin-4-yl)benzyl)-1,2,4-oxadiazole-3-carboxamide

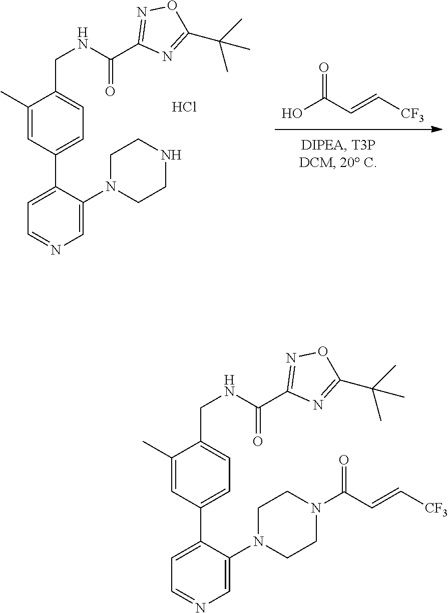 [SMILES: Cc1cc(-c2ccncc2N2CCNCC2)ccc1CNC(=O)c1noc(C(C)(C)C)n1 ; Cl ; O=C(O)/C=C/C(F)(F)F ; Cc1cc(-c2ccncc2N2CCN(C(=O)/C=C/C(F)(F)F)CC2)ccc1CNC(=O)c1noc(C(C)(C)C)n1]

A flask was charged with 5-tert-butyl-N-[[2-methyl-4-(3-piperazin-1-yl-4-pyridyl)phenyl]methyl]-1,2,4-oxadiazole-3-carboxamide hydrochloride (55 mg, 117 μmol), (E)-4,4,4-trifluorobut-2-enoic acid (33 mg, 234 μmol) and DCM (1 mL). Then, DIPEA (60 mg, 467 μmol, 81 μL) was added followed by T3P (186 mg, 292 μmol, 50% purity). After stirring at room temperature overnight, the mixture was diluted with water and extracted with ethyl acetate (×2). The combined organic extracts were washed with brine, dried with Na₂SO₄, filtered, and the filtrate was concentrated. The residual material was purified on acidic prep-HPLC (10-95%) Rt=8.8 min to give (E)-5-(tert-butyl)-N-(2-methyl-4-(3-(4-(4,4,4-trifluorobut-2-enoyl)piperazin-1-yl)pyridin-4-yl)benzyl)-1,2,4-oxadiazole-3-carboxamide trifluoroacetate as a pale-yellow solid (35 mg, 43% yield). LCMS: m/z=557.5 (M+H⁺). ¹H NMR (DMSO-d₆, 400 MHz): δ=9.47 (t, J=6.0 Hz, 1H), 8.51 (d, J=5.5 Hz, 1H), 8.44 (s, 1H), 7.64-7.72 (m, 3H), 7.31-7.42 (m, 2H), 6.71-6.80 (m, 1H), 4.51 (d, J=6.0 Hz, 2H), 3.44-3.54 (m, 2H), 2.96 (br s, 2H), 2.91 (br s, 2H), 2.41 (s, 3H) 1.43 (s, 9H).

### Example 59: Synthesis of 5-(tert-butyl)-N-(2-methyl-4-(3-(4-(oxirane-2-carbonyl)piperazin-1-yl)pyridin-4-yl)benzyl)-1,2,4-oxadiazole-3-carboxamide

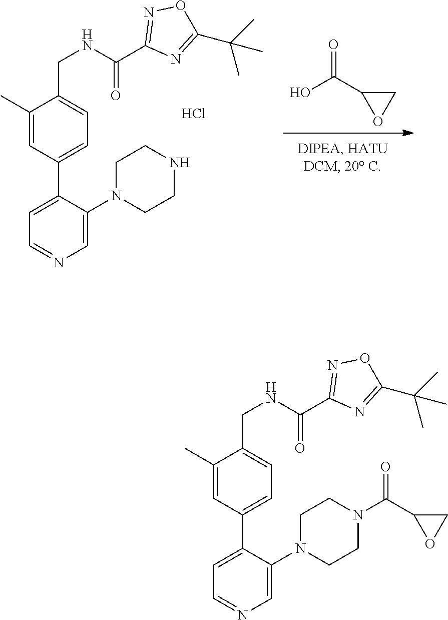 [SMILES: Cc1cc(-c2ccncc2N2CCNCC2)ccc1CNC(=O)c1noc(C(C)(C)C)n1 ; Cl ; O=C(O)C1CO1 ; Cc1cc(-c2ccncc2N2CCN(C(=O)C3CO3)CC2)ccc1CNC(=O)c1noc(C(C)(C)C)n1]

To a solution of 5-(tert-butyl)-N-(2-methyl-4-(3-(piperazin-1-yl)pyridin-4-yl)benzyl)-1,2,4-oxadiazole-3-carboxamide hydrochloride (60 mg, 127 μmol) in DCM (15 mL) was added DIPEA (33 mg, 255 μmol) at 20° C. Then oxirane-2-carboxylic acid (22 mg, 255 μmol) and HATU (97.13 mg, 254.78 μmol, 2.0 eq) were added to the mixture at 20° C. slowly. The mixture was stirred at 20° C. for 5 hours. The mixture was poured into water (50 mL) and extracted with DCM (50 mL×3). The combined organic layers were washed with brine (100 mL), dried over Na₂SO₄, filtered and concentrated under vacuum to give the crude, which was purified by Prep-HPLC (Column: Welch Xtimate C18 150×25 mm×5 m; Condition: water (10 mM NH₄HCO₃)-ACN, Begin B 33, End B 63; Gradient Time (min): 10; 100% B Hold Time (min): 2; Flow Rate (ml/min): 25) to give 5-(tert-butyl)-N-(2-methyl-4-(3-(4-(oxirane-2-carbonyl)piperazin-1-yl)pyridin-4-yl)benzyl)-1,2,4-oxadiazole-3-carboxamide as a yellow solid (22 mg, 34% yield). LCMS: m/z=M+H⁺: 505.3. ¹H NMR: (400 MHz, CDCl₃) δ: 8.39-8.31 (m, 1H), 8.29 (s, 1H), 7.53-7.47 (m, 2H), 7.36 (d, J=7.6 Hz, 1H), 7.20-7.09 (m, 2H), 4.70 (d, J=6.0 Hz, 2H), 3.65-3.50 (m, 5H), 3.00-2.85 (m, 6H), 2.41 (s, 3H), 1.46 (s, 9H).

### Example 60: Synthesis of 5-(tert-butyl)-N-(2-methyl-4-(3-(4-propioloylpiperazin-1-yl)pyridin-4-yl)benzyl)-1,2,4-oxadiazole-3-carboxamide

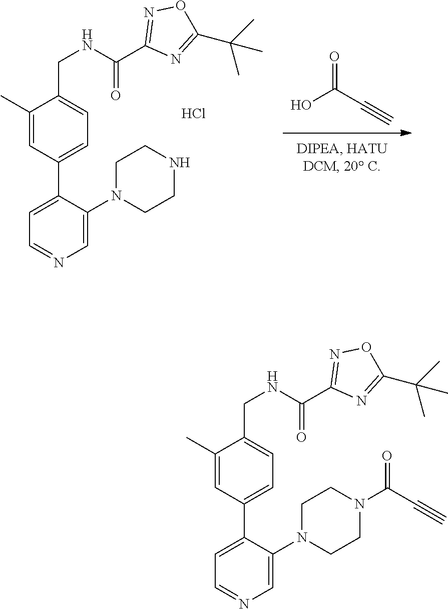 [0753] 1. Synthesis of 5-(tert-butyl)-N-(2-methyl-4-(3-(4-propioloylpiperazin-1-yl)pyridin-4-yl)benzyl)-1,2,4-oxadiazole-3-carboxamide was similar to that of 5-(tert-butyl)-N-(2-methyl-4-(3-(4-(oxirane-2-carbonyl)piperazin-1-yl)pyridin-4-yl)benzyl)-1,2,4-oxadiazole-3-carboxamide in Example 59. The crude material was purified by Prep-HPLC (Column: Welch Xtimate C18 150×25 mm×5 μm; Condition: water (10 mM NH₄HCO₃)-ACN, Begin B 34, End B 64, Gradient Time (min) 10, 100% B Hold Time (min) 2, Flow Rate (mL/min) 25) to give 5-(tert-butyl)-N-(2-methyl-4-(3-(4-propioloylpiperazin-1-yl)pyridin-4-yl)benzyl)-1,2,4-oxadiazole-3-carboxamide as a white solid (62 mg, 40% yield). LCMS: m/z=M+H⁺: 487.3. ¹H NMR: (400 MHz, DMSO-d₆) δ=9.39 (t, J=6.0 Hz, 1H), 8.29-8.25 (m, 2H), 7.56-7.53 (m, 2H), 7.29 (d, J=8.0 Hz, 1H), 7.20 (d, J=4.8 Hz, 1H), 4.53 (s, 1H), 4.46 (d, J=6.0 Hz, 2H), 3.61-3.59 (m, 2H), 3.40-3.38 (m, 2H), 2.90-2.88 (m, 2H), 2.80-2.78 (m, 2H), 2.35 (s, 3H), 1.40 (s, 9H).

### Example 61: Synthesis of (E)-5-(tert-butyl)-N-(4-(3-(4-(4-(dimethylamino)but-2-enoyl)piperazin-1-yl)pyridin-4-yl)-2-methylbenzyl)-1,2,4-oxadiazole-3-carboxamide

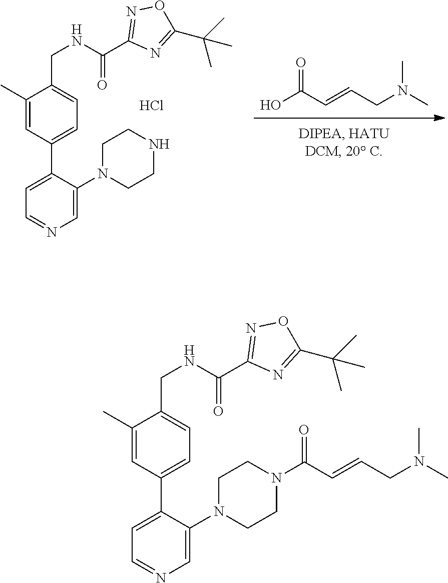 [0755] 1. Synthesis of (E)-5-(tert-butyl)-N-(4-(3-(4-(4-(dimethylamino)but-2-enoyl)piperazin-1-yl)pyridin-4-yl)-2-methylbenzyl)-1,2,4-oxadiazole-3-carboxamide was similar to that of 5-(tert-butyl)-N-(2-methyl-4-(3-(4-(oxirane-2-carbonyl)piperazin-1-yl)pyridin-4-yl)benzyl)-1,2,4-oxadiazole-3-carboxamide in Example 59. The crude material was purified by prep-HPLC (Column: Welch Xtimate C18 150×25 mm×5 μm; Condition: water (10 mM NH₄HCO₃)-ACN, Begin B 31 End B 61, Gradient Time (min) 15, 100% B Hold Time (min) 2, Flow Rate (mL/min): 25) to give (E)-5-(tert-butyl)-N-(4-(3-(4-(4-(dimethylamino)but-2-enoyl)piperazin-1-yl)pyridin-4-yl)-2-methylbenzyl)-1,2,4-oxadiazole-3-carboxamide as a white solid (34 mg, 39% yield). LCMS: m/z=M+H⁺: 546.4. ¹H NMR: (500 MHz, DMSO-d₆) δ ppm=9.43 (t, J=6.0 Hz, 1H), 8.30-8.27 (m, 2H), 7.59-7.56 (m, 2H), 7.32 (d, J=7.5 Hz, 1H), 7.22 (d, J=4.5 Hz, 1H), 6.59-6.56 (m, 2H), 4.49 (d, J=6.0 Hz, 2H), 3.48 (s, 4H), 2.99 (d, J=4.5 Hz, 2H), 2.83 (s, 4H), 2.38 (s, 3H), 2.12 (s, 6H), 1.43 (s, 9H).

### Example 62: Synthesis of N-(4-(3-(4-(but-2-ynoyl)piperazin-1-yl)pyridin-4-yl)-2-methylbenzyl)-5-(tert-butyl)-1,2,4-oxadiazole-3-carboxamide

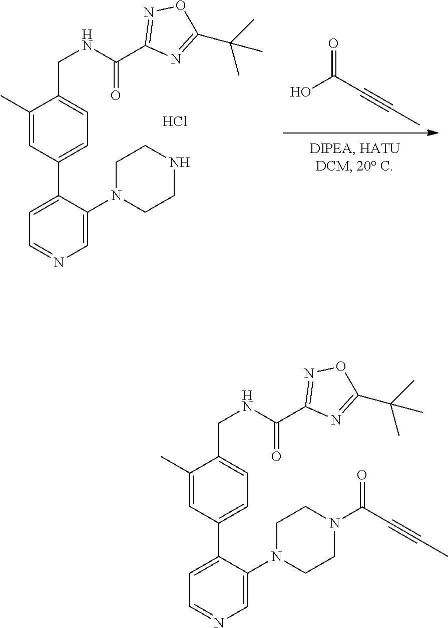 [0757] 1. Synthesis of N-(4-(3-(4-(but-2-ynoyl)piperazin-1-yl)pyridin-4-yl)-2-methylbenzyl)-5-(tert-butyl)-1,2,4-oxadiazole-3-carboxamide was similar to that of 5-(tert-butyl)-N-(2-methyl-4-(3-(4-(oxirane-2-carbonyl)piperazin-1-yl)pyridin-4-yl)benzyl)-1,2,4-oxadiazole-3-carboxamide in Example 59. The crude material was purified by Prep-HPLC (Column: Agela Dura Shell C18 150×25 mm×5 μm; Condition: water (0.05% NH₃H₂O+10 mM NH₄HCO₃)-ACN, Begin B 35, End B 65, Gradient Time (min) 10, 100% B Hold Time (min) 2, Flow Rate (mL/min) 25.) to give N-(4-(3-(4-(but-2-ynoyl)piperazin-1-yl)pyridin-4-yl)-2-methylbenzyl)-5-(tert-butyl)-1,2,4-oxadiazole-3-carboxamide as a white solid (22 mg, 25% yield). LCMS: m/z=M+H⁺: 501.4. ¹H NMR: (500 MHz, METHANOL-d₄) δ=8.28-8.25 (m, 2H), 7.60-7.55 (m, 2H), 7.44 (d, J=8.5 Hz, 1H), 7.30 (d, J=5.0 Hz, 1H), 4.67 (s, 2H), 3.77-3.69 (m, 2H), 3.58-3.51 (m, 2H), 3.00-2.94 (m, 2H), 2.91 (t, J=5.0 Hz, 2H), 2.48 (s, 3H), 2.03 (s, 3H), 1.50 (s, 9H).

### Example 63: Synthesis of N-(4-(3-(4-acryloylpiperazin-1-yl)pyridin-4-yl)-2-methylbenzyl)-5-(tert-butyl)-1,2,4-oxadiazole-3-carboxamide

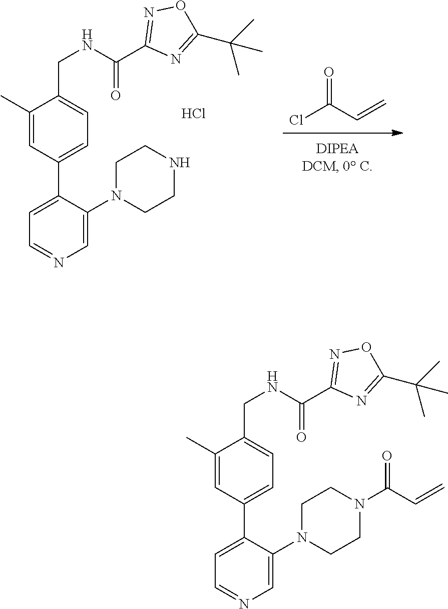

To a solution of 5-(tert-butyl)-N-(2-methyl-4-(3-(piperazin-1-yl)pyridin-4-yl)benzyl)-1,2,4-oxadiazole-3-carboxamide hydrochloride (130 mg, 276 μmol) in DCM (20 mL) was added DIPEA (71 mg, 552 μmol) and acryloyl chloride (25 mg, 276 μmol) at 0° C. The mixture was stirred at 0° C. for 10 minutes. The mixture was quenched with MeOH (1 mL) and concentrated under vacuum. The residue was purified by prep-HPLC (Column: Welch Xtimate C18 150×25 mm×5 μm; Condition: water (10 mM NH₄HCO₃)-ACN, Begin B 32 End B 62, Gradient Time (min) 15, 100% B Hold Time (min) 2, Flow Rate (mL/min): 25) to give N-(4-(3-(4-acryloylpiperazin-1-yl)pyridin-4-yl)-2-methylbenzyl)-5-(tert-butyl)-1,2,4-oxadiazole-3-carboxamide as a white solid (34 mg, 25% yield). LCMS: m/z=M+H⁺: 489.4. ¹H NMR: (500 MHz, DMSO-d₆) δ ppm=9.43 (t, J=6.0 Hz, 1H), 8.32-8.26 (m, 2H), 7.60-7.56 (m, 2H), 7.33 (d, J=8.5 Hz, 1H), 7.22 (d, J=5.0 Hz, 1H), 6.77 (dd, J₁=10.5 Hz, J₂=17.0 Hz, 1H), 6.10 (dd, J₁=2.5 Hz, J₂=16.5 Hz, 1H), 5.69-5.63 (m, 1H), 4.49 (d, J=6.0 Hz, 2H), 3.50 (s, 4H), 2.84 (d, J=5.5 Hz, 4H), 2.38 (s, 3H), 1.43 (s, 9H).

### Example 64: (E)-5-(tert-butyl)-N-(4-(3-(4-(4-(3-fluoroazetidin-1-yl)but-2-enoyl)piperazin-1-yl)pyridin-4-yl)-2-methylbenzyl)-1,2,4-oxadiazole-3-carboxamide

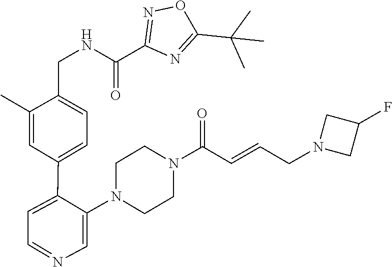 [SMILES: Cc1cc(-c2ccncc2N2CCN(C(=O)/C=C/CN3CC(F)C3)CC2)ccc1CNC(=O)c1noc(C(C)(C)C)n1]

### 1. Synthesis of (E)-N-(4-(3-(4-(4-bromobut-2-enoyl)piperazin-1-yl)pyridin-4-yl)-2-methylbenzyl)-5-(tert-butyl)-1,2,4-oxadiazole-3-carboxamide

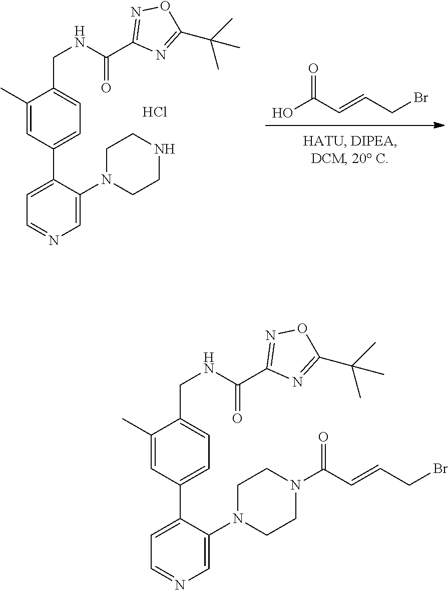 [SMILES: Cc1cc(-c2ccncc2N2CCNCC2)ccc1CNC(=O)c1noc(C(C)(C)C)n1 ; Cl ; O=C(O)/C=C/CBr ; Cc1cc(-c2ccncc2N2CCN(C(=O)/C=C/CBr)CC2)ccc1CNC(=O)c1noc(C(C)(C)C)n1]

To a solution of 5-tert-butyl-N-[[2-methyl-4-(3-piperazin-1-yl-4-pyridyl)phenyl]methyl]-1,2,4-oxadiazole-3-carboxamide hydrochloride (2.2 g, 4.7 mmol) in DCM (120 mL) was added DIPEA (1.8 g, 14 mmol, 2.4 mL) at 20° C. Then (E)-4-bromobut-2-enoic acid (848 mg, 5.1 mmol) and HATU (2.0 g, 5.1 mmol) was added to the mixture at 20° C. slowly. The mixture was stirred at 20° C. for 2 h. The mixture was concentrated under vacuum to give N-[[4-[3-[4-[(E)-4-bromobut-2-enoyl]piperazin-1-yl]-4-pyridyl]-2-methyl-phenyl]methyl]-5-tert-butyl-1,2,4-oxadiazole-3-carboxamide as a yellow oil (2.7 g, crude), which was used for the next step directly without further purification. LCMS: m/z=M+H⁺: 581.2, 583.2.

### 2. Synthesis of (E)-5-(tert-butyl)-N-(4-(3-(4-(4-(3-fluoroazetidin-1-yl)but-2-enoyl)piperazin-1-yl)pyridin-4-yl)-2-methylbenzyl)-1,2,4-oxadiazole-3-carboxamide

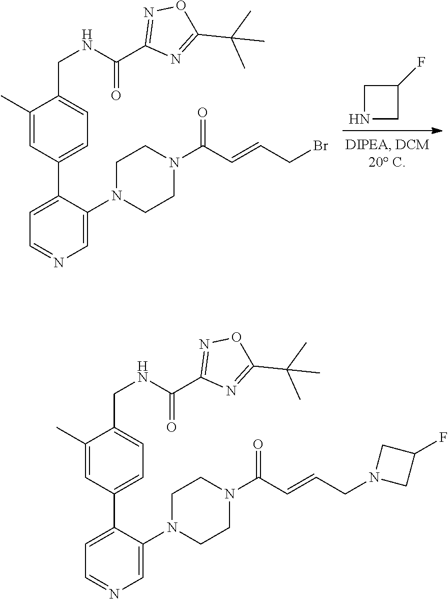 [SMILES: Cc1cc(-c2ccncc2N2CCN(C(=O)/C=C/CBr)CC2)ccc1CNC(=O)c1noc(C(C)(C)C)n1 ; FC1CNC1 ; Cc1cc(-c2ccncc2N2CCN(C(=O)/C=C/CN3CC(F)C3)CC2)ccc1CNC(=O)c1noc(C(C)(C)C)n1]

To a solution of (E)-N-(4-(3-(4-(4-bromobut-2-enoyl)piperazin-1-yl)pyridin-4-yl)-2-methylbenzyl)-5-(tert-butyl)-1,2,4-oxadiazole-3-carboxamide (200 mg, 344 μmol) in DCM (30 mL) was added DIPEA (445 mg, 3.44 mmol) at 20° C. Then 3-fluoroazetidine (959 mg, 8.60 mmol) was added into this mixture at 20° C. Then the mixture was stirred at 20° C. for 2 h. The mixture was poured into water (50 mL) and extracted with DCM (50 mL×3). The combined organic layers were washed with brine (100 mL), dried over Na₂SO₄, filtered, and concentrated under vacuum to give crude product. This material was purified by prep-HPLC (Column: Phenomenex Synergi C18 150×30 mm×4 μm; Condition: water(0.225% FA)-ACN, Begin B 17, End B 17; Gradient Time (min): 10; 100% B Hold Time (min): 2; Flow Rate (ml/min): 25) to give (E)-5-(tert-butyl)-N-(4-(3-(4-(4-(3-fluoroazetidin-1-yl)but-2-enoyl)piperazin-1-yl)pyridin-4-yl)-2-methylbenzyl)-1,2,4-oxadiazole-3-carboxamide as a yellow solid (33 mg, 16% yield). LCMS: m/z=M+H⁺: 576.3. ¹H NMR: (400 MHz, METHANOL-d₄) δ=8.26-8.20 (m, 2H), 7.58-7.52 (m, 2H), 7.40 (d, J=8.4 Hz, 1H), 7.27 (d, J=4.8 Hz, 1H), 6.66-6.52 (m, 2H), 4.62 (s, 2H), 4.01-3.44 (m, 11H), 2.93-2.85 (m, 4H), 2.44 (s, 3H), 1.47-1.45 (m, 9H).

### Example 65: Synthesis of (E)-5-(tert-butyl)-N-(2-methyl-4-(3-(4-(4-(4-methylpiperazin-1-yl)but-2-enoyl)piperazin-1-yl)pyridin-4-yl)benzyl)-1,2,4-oxadiazole-3-carboxamide

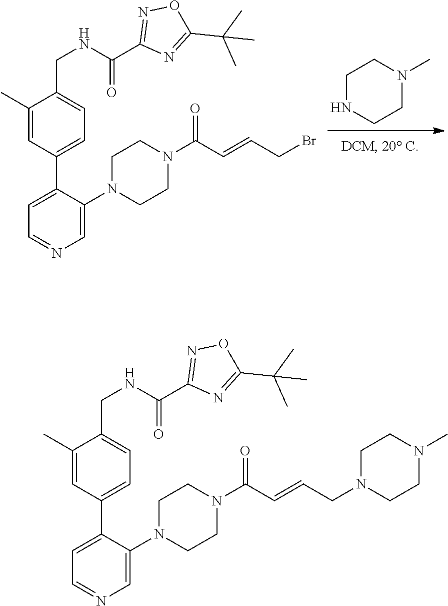

To a solution of (E)-N-(4-(3-(4-(4-bromobut-2-enoyl)piperazin-1-yl)pyridin-4-yl)-2-methylbenzyl)-5-(tert-butyl)-1,2,4-oxadiazole-3-carboxamide (200 mg, 344 μmol) in DCM (20 mL) was added 1-methylpiperazine (52 mg, 516 μmol) at 20° C. The mixture was stirred at 20° C. for 2 h. The mixture was poured into water (50 mL) and extracted with DCM (50 mL×3). The combined organic layers were washed with brine (100 mL), dried over Na₂SO₄, filtered, and concentrated under vacuum to give crude product. This material was purified by Prep-HPLC (Column: Welch Xtimate C18 150×25 mm×5 μm; Condition: water(10 mM NH₄HCO₃)-ACN, Begin B 28, End B 58; Gradient Time (min): 10; 100% B Hold Time (min): 2; Flow Rate (ml/min): 25) to give (E)-5-(tert-butyl)-N-(2-methyl-4-(3-(4-(4-(4-methylpiperazin-1-yl)but-2-enoyl)piperazin-1-yl)pyridin-4-yl)benzyl)-1,2,4-oxadiazole-3-carboxamide as a yellow solid (82 mg, 40% yield). LCMS: m/z=M+H⁺: 601.4. ¹H NMR: (400 MHz, METHANOL-d₄) δ=8.28-8.23 (m, 2H), 7.59-7.54 (m, 2H), 7.47-7.39 (m, 1H), 7.29 (d, J=5.2 Hz, 1H), 6.81-6.69 (m, 1H), 6.65-6.54 (m, 1H), 4.65 (s, 2H), 3.57 (s, 4H), 3.18 (d, J=6.0 Hz, 2H), 2.92 (s, 4H), 2.75-2.22 (m, 14H), 1.48 (s, 9H).

### Example 66: Synthesis of (E)-N-(4-(3-(4-(4-(azetidin-1-yl)but-2-enoyl)piperazin-1-yl)pyridin-4-yl)-2-methylbenzyl)-5-(tert-butyl)-1,2,4-oxadiazole-3-carboxamide

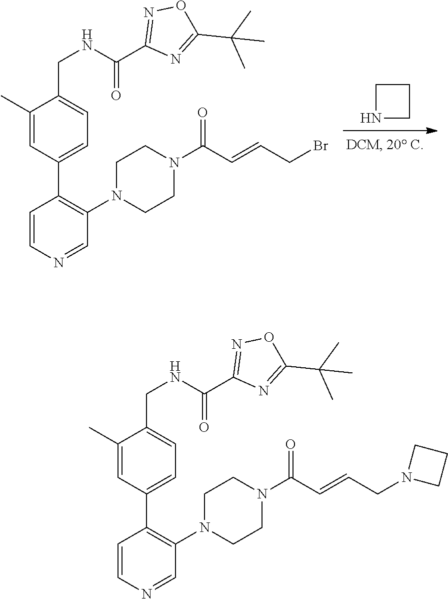 [0767] 1. Synthesis of (E)-N-(4-(3-(4-(4-(azetidin-1-yl)but-2-enoyl)piperazin-1-yl)pyridin-4-yl)-2-methylbenzyl)-5-(tert-butyl)-1,2,4-oxadiazole-3-carboxamide was similar to that of (E)-5-(tert-butyl)-N-(2-methyl-4-(3-(4-(4-(4-methylpiperazin-1-yl)but-2-enoyl)piperazin-1-yl)pyridin-4-yl)benzyl)-1,2,4-oxadiazole-3-carboxamide in Example 65. The crude material was purified by Prep-HPLC (Column: Phenomenex Synergi C18 150×30 mm×4 μm; Condition: water(0.225% FA)-ACN, Begin B 5, End B 35; Gradient Time (min): 11; 100% B Hold Time (min): 2; Flow Rate (ml/min): 25) to give (E)-N-(4-(3-(4-(4-(azetidin-1-yl)but-2-enoyl)piperazin-1-yl)pyridin-4-yl)-2-methylbenzyl)-5-(tert-butyl)-1,2,4-oxadiazole-3-carboxamide as a yellow solid (65 mg, 23% yield). LCMS: m/z=M+H⁺: 558.4. ¹H NMR: (400 MHz, METHANOL-d₄) δ=8.43 (s, 1H), 8.26 (d, J=4.8 Hz, 1H), 7.60-7.54 (m, 2H), 7.42 (d, J=8.4 Hz, 1H), 7.29 (d, J=4.8 Hz, 1H), 6.77 (d, J=15.2 Hz, 1H), 6.59-6.48 (m, 1H), 4.64 (s, 2H), 4.07 (t, J=8.0 Hz, 4H), 3.88 (d, J=6.4 Hz, 2H), 3.59-3.57 (m, 4H), 2.95-2.92 (m, 4H), 2.55-2.44 (m, 5H), 1.49 (s, 9H).

### Example 67: Synthesis of (E)-5-(tert-butyl)-N-(2-methyl-4-(3-(4-(4-morpholinobut-2-enoyl)piperazin-1-yl)pyridin-4-yl)benzyl)-1,2,4-oxadiazole-3-carboxamide

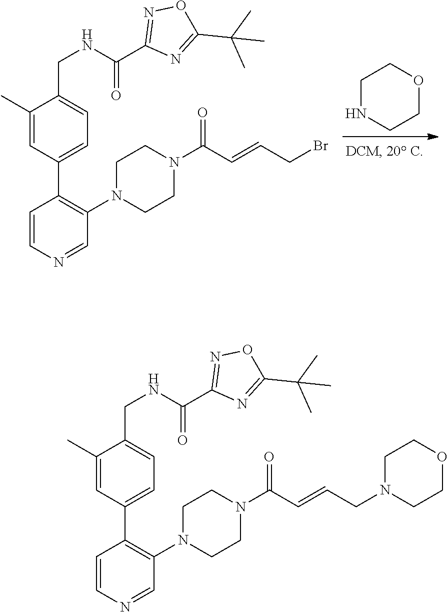 [SMILES: Cc1cc(-c2ccncc2N2CCN(C(=O)/C=C/CBr)CC2)ccc1CNC(=O)c1noc(C(C)(C)C)n1 ; C1COCCN1 ; Cc1cc(-c2ccncc2N2CCN(C(=O)/C=C/CN3CCOCC3)CC2)ccc1CNC(=O)c1noc(C(C)(C)C)n1] [0769] 1. Synthesis of (E)-5-(tert-butyl)-N-(2-methyl-4-(3-(4-(4-morpholinobut-2-enoyl)piperazin-1-yl)pyridin-4-yl)benzyl)-1,2,4-oxadiazole-3-carboxamide was similar to that of (E)-5-(tert-butyl)-N-(2-methyl-4-(3-(4-(4-(4-methylpiperazin-1-yl)but-2-enoyl)piperazin-1-yl)pyridin-4-yl)benzyl)-1,2,4-oxadiazole-3-carboxamide in Example 65. The crude material was purified by Prep-HPLC (Column: Welch Xtimate C18 150×25 mm×5 m; Condition: water(10 mM NH₄HCO₃)-ACN, Begin B 30, End B 60; Gradient Time (min): 10; 100% B Hold Time (min): 2; Flow Rate (ml/min): 25) to give (E)-5-(tert-butyl)-N-(2-methyl-4-(3-(4-(4-morpholinobut-2-enoyl)piperazin-1-yl)pyridin-4-yl)benzyl)-1,2,4-oxadiazole-3-carboxamide as a yellow solid (79 mg, 31% yield). LCMS: m/z=M+H⁺: 588.3. ¹H NMR: (400 MHz, MeOH-d₄) δ: 8.27-8.22 (m, 2H), 7.60-7.55 (m, 2H), 7.45-7.39 (m, 1H), 7.28 (d, J=5.2 Hz, 1H), 6.80-6.69 (m, 1H), 6.64-6.54 (m, 1H), 4.64 (s, 2H), 3.73-3.66 (m, 4H), 3.57 (s, 4H), 3.17 (d, J=5.6 Hz, 2H), 2.96-2.90 (m, 4H), 2.49-2.41 (m, 7H), 1.49 (s, 9H).

### Example 68: Synthesis of (E)-5-(tert-butyl)-N-(2-methyl-4-(3-(4-(4-(methylamino)but-2-enoyl)piperazin-1-yl)pyridin-4-yl)benzyl)-1,2,4-oxadiazole-3-carboxamide

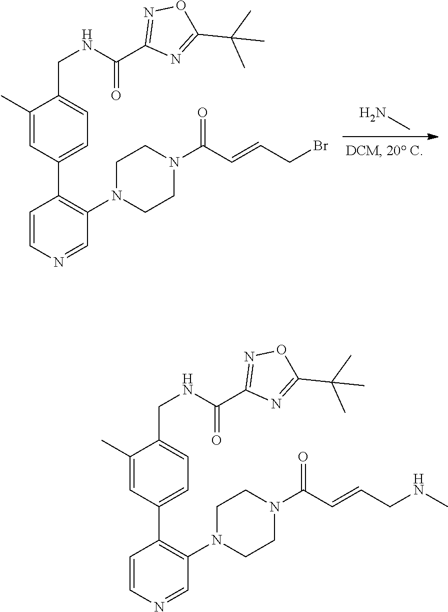 [0771] 1. Synthesis of (E)-5-(tert-butyl)-N-(2-methyl-4-(3-(4-(4-(methylamino)but-2-enoyl)piperazin-1-yl)pyridin-4-yl)benzyl)-1,2,4-oxadiazole-3-carboxamide was similar to that of (E)-5-(tert-butyl)-N-(2-methyl-4-(3-(4-(4-(4-methylpiperazin-1-yl)but-2-enoyl)piperazin-1-yl)pyridin-4-yl)benzyl)-1,2,4-oxadiazole-3-carboxamide in Example 65. The crude material was purified by Prep-HPLC (Column: Welch Xtimate C18 150×25 mm×5 m; Condition: water(10 mM NH₄HCO₃)-ACN, Begin B 30, End B 60; Gradient Time (min): 10; 100% B Hold Time (min): 2; Flow Rate (ml/min): 25) to give (E)-5-(tert-butyl)-N-(2-methyl-4-(3-(4-(4-(methylamino)but-2-enoyl)piperazin-1-yl)pyridin-4-yl)benzyl)-1,2,4-oxadiazole-3-carboxamide as a yellow solid (91 mg, 49% yield). LCMS: m/z=M+H⁺: 532.3. ¹H NMR: (400 MHz, MeOH-d₄) δ: 8.28-8.23 (m, 2H), 7.63-7.58 (m, 2H), 7.45-7.42 (m, 1H), 7.29 (d, J=5.2 Hz, 1H), 6.81-6.67 (m, 1H), 6.64-6.47 (m, 1H), 4.64 (s, 2H), 3.58 (s, 4H), 3.43 (d, J=6.0 Hz, 2H), 2.96-2.90 (m, 4H), 2.45 (d, J=4.0 Hz, 6H), 1.49 (s, 9H).

### Example 69: (E)-5-(tert-butyl)-N-(4-(3-cyano-5-(4-(4-(dimethylamino)but-2-enoyl)piperazin-1-yl)pyridin-4-yl)-2-methylbenzyl)-1,2,4-oxadiazole-3-carboxamide

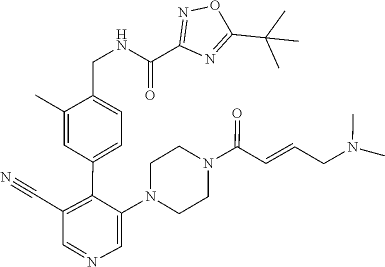 [SMILES: Cc1cc(-c2c(C#N)cncc2N2CCN(C(=O)/C=C/CN(C)C)CC2)ccc1CNC(=O)c1noc(C(C)(C)C)n1]

### 1. Synthesis of tert-butyl 4-(4-chloro-5-cyanopyridin-3-yl)piperazine-1-carboxylate

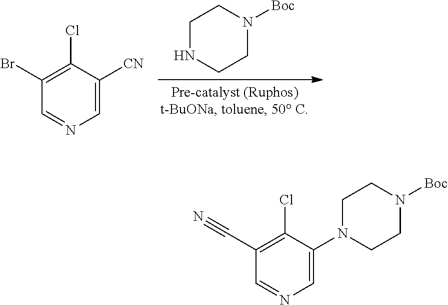 [SMILES: N#Cc1cncc(Br)c1Cl ; CC(C)(C)OC(=O)N1CCNCC1 ; CC(C)(C)OC(=O)N1CCN(c2cncc(C#N)c2Cl)CC1]

To a solution of 5-bromo-4-chloronicotinonitrile (300 mg, 1.38 mmol) in toluene (10 mL) was added tert-butyl piperazine-1-carboxylate (231 mg, 1.24 mmol) and sodium tert-butoxide (265 mg, 2.76 mmol) at 15° C. Then Pre-catalyst (Ruphos) (231 mg, 276 μmol) was added at 15° C. The mixture was stirred at 50° C. for 5 hours under N₂. The mixture was filtered and concentrated under vacuum to give crude product. This material was purified by pre-HPLC (Column: Welch Xtimate C18 150×25 mm×5 μm; Condition: water (10 mM NH₄HCO₃)-ACN, Begin B 40, End B 70, Gradient Time (min) 10, 100% B Hold Time (min) 2, Flow Rate (mL/min) 25.) to give the tert-butyl 4-(4-chloro-5-cyanopyridin-3-yl)piperazine-1-carboxylate as a yellow solid (55 mg, 12% yield). LCMS: m/z=M+H⁺: 323.3. 2. Synthesis of tert-butyl 4-(4-(4-((5-(tert-butyl)-1,2,4-oxadiazole-3-carboxamido)methyl)-3-methylphenyl)-5-cyanopyridin-3-yl)piperazine-1-carboxylate

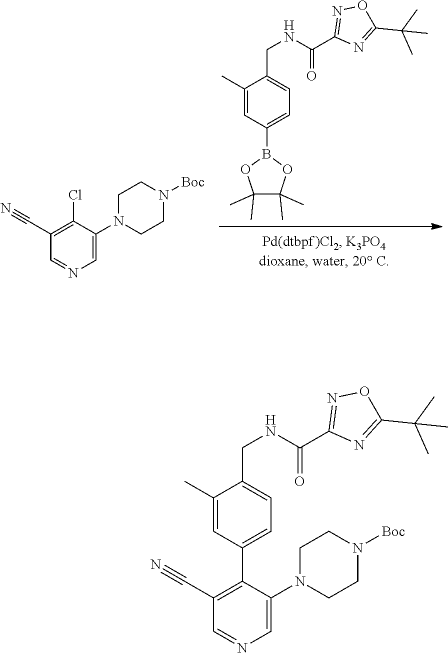 [SMILES: CC(C)(C)OC(=O)N1CCN(c2cncc(C#N)c2Cl)CC1 ; Cc1cc(B2OC(C)(C)C(C)(C)O2)ccc1CNC(=O)c1noc(C(C)(C)C)n1 ; Cc1cc(-c2c(C#N)cncc2N2CCN(C(=O)OC(C)(C)C)CC2)ccc1CNC(=O)c1noc(C(C)(C)C)n1]

To a solution of tert-butyl 4-(4-chloro-5-cyanopyridin-3-yl)piperazine-1-carboxylate (50 mg, 155 μmol) in dioxane (5 mL) and water (1 mL) was added 5-(tert-butyl)-N-(2-methyl-4-(4,4,5,5-tetramethyl-1,3,2-dioxaborolan-2-yl)benzyl)-1,2,4-oxadiazole-3-carboxamide (62 mg, 155 μmol) and K₃PO₄ (99 mg, 465 μmol) at 20° C. Then Pd(dtbpf)Cl₂ (20 mg, 31 μmol) was added at 20° C. The mixture was stirred at 20° C. for 3 hours under N₂. The mixture was filtered, and the filtrate was concentrated under vacuum. The crude material was purified by chromatography column on silica gel (petroleum ether/ethyl acetate=1/0 to 2/1) to give tert-butyl 4-(4-(4-((5-(tert-butyl)-1,2,4-oxadiazole-3-carboxamido)methyl)-3-methylphenyl)-5-cyanopyridin-3-yl)piperazine-1-carboxylate as a yellow solid (80 mg, 76% yield). LCMS: m/z=M+H⁺: 560.5.

### 3. Synthesis of 5-(tert-butyl)-N-(4-(3-cyano-5-(piperazin-1-yl)pyridin-4-yl)-2-methylbenzyl)-1,2,4-oxadiazole-3-carboxamide hydrochloride

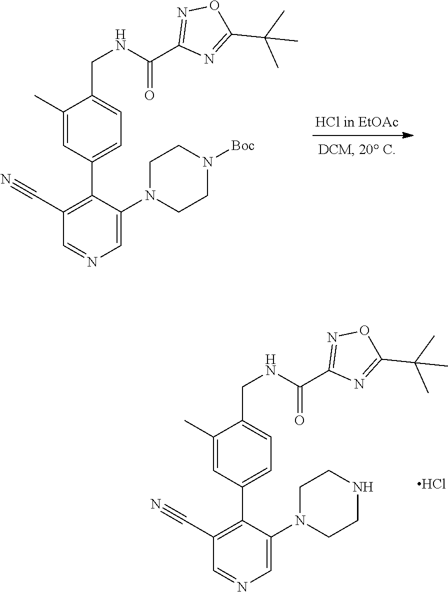 [SMILES: Cc1cc(-c2c(C#N)cncc2N2CCN(C(=O)OC(C)(C)C)CC2)ccc1CNC(=O)c1noc(C(C)(C)C)n1 ; Cc1cc(-c2c(C#N)cncc2N2CCNCC2)ccc1CNC(=O)c1noc(C(C)(C)C)n1 ; Cl]

To a solution of tert-butyl 4-(4-(4-((5-(tert-butyl)-1,2,4-oxadiazole-3-carboxamido)methyl)-3-methylphenyl)-5-cyanopyridin-3-yl)piperazine-1-carboxylate (80 mg, 143 μmol) in DCM (5 mL) was added an HCl solution in ethyl acetate (4 M, 5 mL) and the reaction mixture was stirred at 20° C. for 1 hour. The mixture was concentrated under vacuum to give 5-(tert-butyl)-N-(4-(3-cyano-5-(piperazin-1-yl)pyridin-4-yl)-2-methylbenzyl)-1,2,4-oxadiazole-3-carboxamide hydrochloride as a yellow solid (65 mg, crude), which was carried forward without further purification. LCMS: m/z=M+H⁺: 460.3. 4. Synthesis of (E)-5-(tert-butyl)-N-(4-(3-cyano-5-(4-(4-(dimethylamino)but-2-enoyl)piperazin-1-yl)pyridin-4-yl)-2-methylbenzyl)-1,2,4-oxadiazole-3-carboxamide

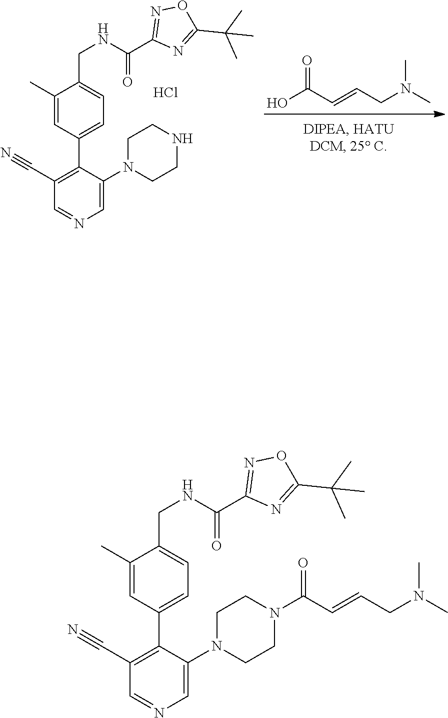

5. Synthesis of (E)-5-(tert-butyl)-N-(4-(3-cyano-5-(4-(4-(dimethylamino)but-2-enoyl)piperazin-1-yl)pyridin-4-yl)-2-methylbenzyl)-1,2,4-oxadiazole-3-carboxamide was similar to that of (E)-5-(tert-butyl)-N-(4-(3-(4-(4-(dimethylamino)but-2-enoyl)piperazin-1-yl)pyridin-4-yl)-2-methylbenzyl)-1,2,4-oxadiazole-3-carboxamide in Example 61. The crude material was purified by prep-HPLC (Column: Welch Xtimate C18 150×25 mm×5 μm; Condition: water (10 mM NH₄HCO₃)-ACN, Begin B 35, End B 65, Gradient Time (min) 10, 100% B Hold Time (min) 2, Flow Rate (mL/min) 25.) to give (E)-5-(tert-butyl)-N-(4-(3-cyano-5-(4-(4-(dimethylamino)but-2-enoyl)piperazin-1-yl)pyridin-4-yl)-2-methylbenzyl)-1,2,4-oxadiazole-3-carboxamide as a yellow solid (45 mg, 65% yield). LCMS: m/z=M+H⁺: 571.4. ¹H NMR: (500 MHz, DMSO-d₆) δ=9.50-9.47 (m, 1H), 8.74-8.67 (m, 1H), 8.59-8.54 (m, 1H), 7.48-7.39 (m, 3H), 6.62-6.52 (m, 2H), 4.54 (d, J=6.0 Hz, 2H), 3.39-3.37 (m, 4H), 2.99 (d, J=5.0 Hz, 2H), 2.83-2.81 (m, 4H), 2.41-2.36 (m, 3H), 2.12 (s, 6H), 1.44 (s, 9H).

### Example 70: (E)-5-(tert-butyl)-N-(4-(3-cyano-5-(4-(4-(dimethylamino)but-2-enoyl)piperazin-1-yl)pyridin-4-yl)-2-methylbenzyl)isoxazole-3-carboxamide

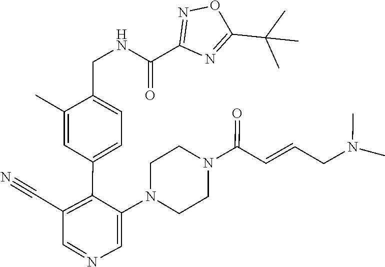 [SMILES: Cc1cc(-c2c(C#N)cncc2N2CCN(C(=O)/C=C/CN(C)C)CC2)ccc1CNC(=O)c1noc(C(C)(C)C)n1] [0780] 1. Synthesis of tert-butyl 4-(4-(4-((5-(tert-butyl)isoxazole-3-carboxamido)methyl)-3-methylphenyl)-5-cyanopyridin-3-yl)piperazine-1-carboxylate

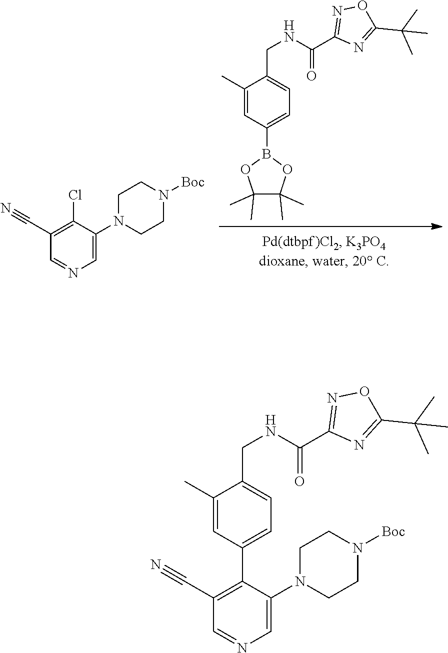 [SMILES: CC(C)(C)OC(=O)N1CCN(c2cncc(C#N)c2Cl)CC1 ; Cc1cc(B2OC(C)(C)C(C)(C)O2)ccc1CNC(=O)c1noc(C(C)(C)C)n1 ; Cc1cc(-c2c(C#N)cncc2N2CCN(C(=O)OC(C)(C)C)CC2)ccc1CNC(=O)c1noc(C(C)(C)C)n1]

2. Synthesis of tert-butyl 4-(4-(4-((5-(tert-butyl)isoxazole-3-carboxamido)methyl)-3-methylphenyl)-5-cyanopyridin-3-yl)piperazine-1-carboxylate was similar to that of tert-butyl 4-(4-(4-((5-(tert-butyl)-1,2,4-oxadiazole-3-carboxamido)methyl)-3-methylphenyl)-5-cyanopyridin-3-yl)piperazine-1-carboxylate in Example 69, Step 2. The crude material was purified by column chromatography on silica gel (petroleum ether/ethyl acetate=1/0 to 2/1) to give tert-butyl 4-(4-(4-((5-(tert-butyl)isoxazole-3-carboxamido)methyl)-3-methylphenyl)-5-cyanopyridin-3-yl)piperazine-1-carboxylate as a yellow solid (100 mg, 61% yield). LCMS: m/z=M+H⁺: 559.5.
3. Synthesis of 5-(tert-butyl)-N-(4-(3-cyano-5-(piperazin-1-yl)pyridin-4-yl)-2-methylbenzyl)isoxazole-3-carboxamide hydrochloride

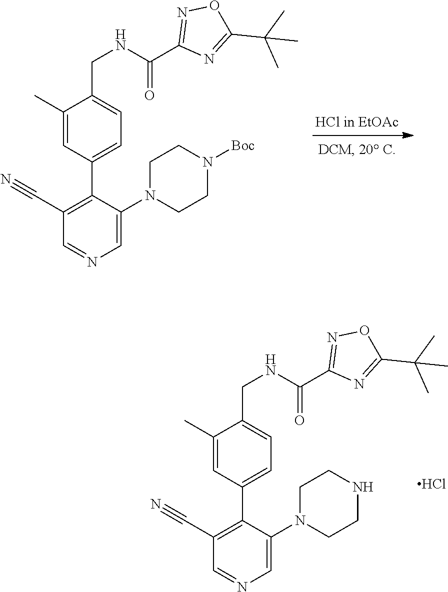 [SMILES: Cc1cc(-c2c(C#N)cncc2N2CCN(C(=O)OC(C)(C)C)CC2)ccc1CNC(=O)c1noc(C(C)(C)C)n1 ; Cc1cc(-c2c(C#N)cncc2N2CCNCC2)ccc1CNC(=O)c1noc(C(C)(C)C)n1 ; Cl]

4. Synthesis of 5-(tert-butyl)-N-(4-(3-cyano-5-(piperazin-3-yl)pyridin-4-yl)-2-methylbenzyl)isoxazole-3-carboxamide hydrochloride was similar to that of 5-(tert-butyl)-N-(4-(3-cyano-5-(piperazin-1-yl)pyridin-4-yl)-2-methylbenzyl)-1,2,4-oxadiazole-3-carboxamide hydrochloride in Example 69, Step 3. The reaction mixture was concentrated under vacuum to give crude 5-(tert-butyl)-N-(4-(3-cyano-5-(piperazin-1-yl)pyridin-4-yl)-2-methylbenzyl)isoxazole-3-carboxamide hydrochloride (80 mg, crude) which was carried forward without further purification. LCMS: m/z=M+H⁺: 459.3. 5. Synthesis of (E)-5-(tert-butyl)-N-(4-(3-cyano-5-(4-(4-(dimethylamino)but-2-enoyl)piperazin-1-yl)pyridin-4-yl)-2-methylbenzyl)isoxazole-3-carboxamide

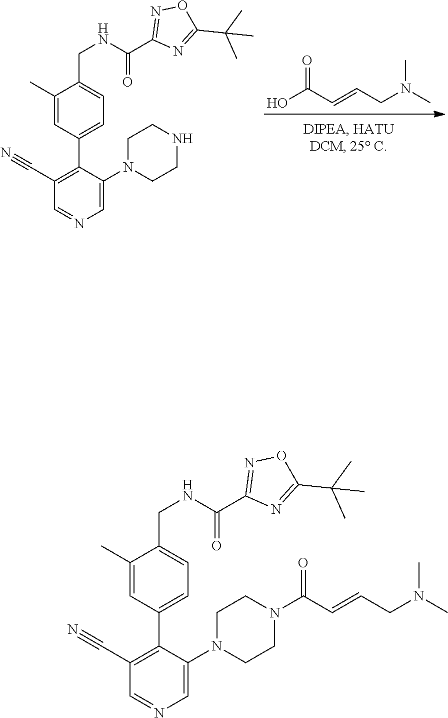

6. Synthesis of (E)-5-(tert-butyl)-N-(4-(3-cyano-5-(4-(4-(dimethylamino)but-2-enoyl)piperazin-1-yl)pyridin-4-yl)-2-methylbenzyl)isoxazole-3-carboxamide was similar to that of (E)-5-(tert-butyl)-N-(4-(3-cyano-5-(4-(4-(dimethylamino)but-2-enoyl)piperazin-1-yl)pyridin-4-yl)-2-methylbenzyl)-1,2,4-oxadiazole-3-carboxamide in Example 69, Step 4. The crude material was purified by prep-HPLC (Column: Welch Xtimate C18 150×25 mm×5 μm; Condition: water (10 mM NH₄HCO₃)-ACN, Begin B 38, End B 68, Gradient Time (min) 10, 100% B Hold Time (min) 2, Flow Rate (mL/min) 25.) to give (E)-5-(tert-butyl)-N-(4-(3-cyano-5-(4-(4-(dimethylamino)but-2-enoyl)piperazin-1-yl)pyridin-4-yl)-2-methylbenzyl)isoxazole-3-carboxamide as a yellow solid (41 mg, 48% yield). LCMS m/z=M+H⁺: 570.4. ¹H NMR: (500 MHz, DMSO-d₆) δ ppm=9.28-9.24 (m, 1H), 8.71 (s, 1H), 8.56 (s, 1H), 7.47-7.43 (m, 2H), 7.40-7.37 (m, 1H), 6.62-6.52 (m, 3H), 4.51 (d, J=6.0 Hz, 2H), 3.38-3.35 (m, 4H), 2.99 (d, J=5.0 Hz, 2H), 2.82 (s, 4H), 2.40 (s, 3H), 2.12 (s, 6H), 1.34 (s, 9H).

### Example 71: (E)-5-(tert-butyl)-N-(4-(3-(4-(4-(dimethylamino)but-2-enoyl)piperazin-1-yl)pyridin-4-yl)-2-methylbenzyl)-N-methyl-1,2,4-oxadiazole-3-carboxamide

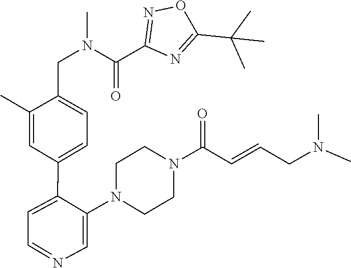

### 1. Synthesis of 1-(4-bromo-2-methylphenyl)-N-methylmethanamine

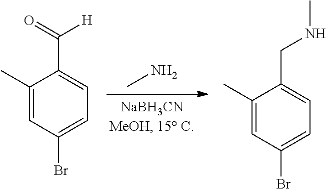

To a solution of methylamine (4.97 g, 52.8 mmol, 7.10 mL) in MeOH (100 mL) was added 4-bromo-2-methyl-benzaldehyde (5.00 g, 25.1 mmol) portion-wise. The reaction was stirred at 15° C. for 2 hours and then NaBH₃CN (3.16 g, 50.2 mmol) was added to the mixture portion-wise. The reaction mixture continued to stir at 15° C. for 32 hours. Water (1 mL) was added, and the reaction mixture was concentrated under vacuum. The crude material was purified by silica gel chromatography (10% MeOH in DCM) to give 1-(4-bromo-2-methyl-phenyl)-N-methyl-methanamine as a semi-solid (3.0 g, 28% yield, 50% purity). This material was carried forward without further purification. LCMS m/z=M+H⁺: 214.0.

### 2. Synthesis of tert-butyl (4-bromo-2-methylbenzyl)(methyl)carbamate

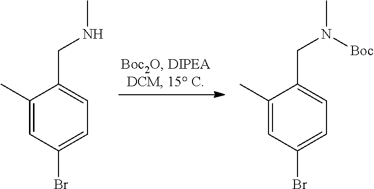

To a solution of 1-(4-bromo-2-methyl-phenyl)-N-methyl-methanamine (3.00 g, 14.0 mmol) in DCM (80 mL) was added DIPEA (5.43 g, 42.0 mmol, 7.34 mL). Then tert-butoxycarbonyl tert-butyl carbonate (3.06 g, 14.0 mmol, 3.22 mL) was added to the above mixture portion-wise at 0° C. slowly. The reaction mixture was stirred at 15° C. for 16 hours. The reaction solvents were concentrated to dryness to give a residue which was purified by silica gel chromatography with eluent (6% ethyl acetate in petroleum ether) to give tert-butyl N-[(4-bromo-2-methyl-phenyl)methyl]-N-methyl-carbamate as a clear oil (2.5 g, 54% yield). LCMS m/z=(M+H-t-Bu)+: 257.9, 259.9.

### 3. Synthesis of tert-butyl methyl(2-methyl-4-(4,4,5,5-tetramethyl-1,3,2-dioxaborolan-2-yl)benzyl)carbamate

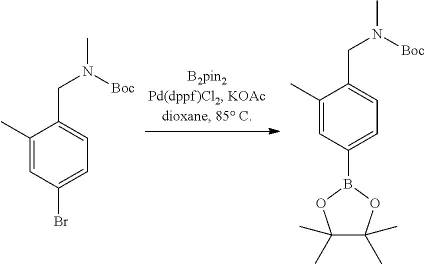 [SMILES: Cc1cc(Br)ccc1CN(C)C(=O)OC(C)(C)C ; Cc1cc(B2OC(C)(C)C(C)(C)O2)ccc1CN(C)C(=O)OC(C)(C)C]

To a solution of tert-butyl N-[(4-bromo-2-methyl-phenyl)methyl]-N-methyl-carbamate (2.50 g, 7.96 mmol) in dioxane (50 mL) was added 4,4,5,5-tetramethyl-2-(4,4,5,5-tetramethyl-1,3,2-dioxaborolan-2-yl)-1,3,2-dioxaborolane (2.22 g, 8.75 mmol) and KOAc (2.34 g, 23.9 mmol). Pd(dppf)Cl₂ (291 mg, 398 μmol) was added to the mixture under nitrogen. The reaction was stirred at 85° C. for 16 hours. The solvents were concentrated to dryness to give a residue which was purified by silica gel chromatography with eluent (10% ethyl acetate in petroleum ether) to give tert-butyl N-methyl-N-[[2-methyl-4-(4,4,5,5-tetramethyl-1,3,2-dioxaborolan-2-yl)phenyl]methyl]carbamate as an oil (2.6 g, 74% yield). LCMS m/z=M+H⁺: 362.2.

### 4. Synthesis of N-methyl-1-(2-methyl-4-(4,4,5,5-tetramethyl-1,3,2-dioxaborolan-2-yl)phenyl)methanamine hydrochloride

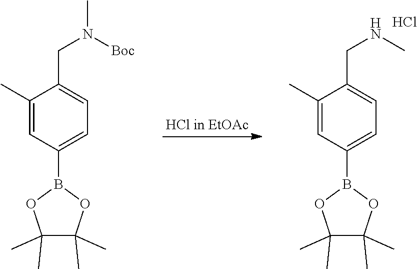

To a solution of tert-butyl methyl(2-methyl-4-(4,4,5,5-tetramethyl-1,3,2-dioxaborolan-2-yl)benzyl)carbamate (300 mg, 830 μmol) in DCM (35 mL) was added an HCl solution in ethyl acetate (4 M, 12 mL) at 25° C. The mixture was stirred at 25° C. for 30 minutes. The reaction mixture was concentrated to get N-methyl-1-(2-methyl-4-(4,4,5,5-tetramethyl-1,3,2-dioxaborolan-2-yl)phenyl)methanamine hydrochloride (200 mg, crude), which was used to next step directly. LCMS: m/z=M+H⁺: 262.2. 5. Synthesis of 5-(tert-butyl)-N-methyl-N-(2-methyl-4-(4,4,5,5-tetramethyl-1,3,2-dioxaborolan-2-yl)benzyl)-1,2,4-oxadiazole-3-carboxamide

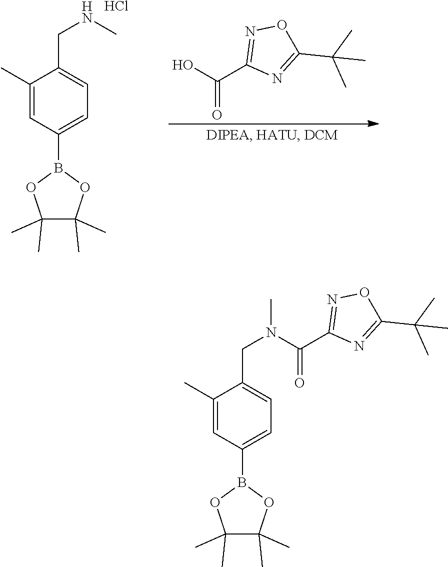

To a solution of N-methyl-1-(2-methyl-4-(4,4,5,5-tetramethyl-1,3,2-dioxaborolan-2-yl)phenyl)methanamine hydrochloride (150 mg, 574 μmol) in DCM (35 mL) was added DIPEA (223 mg, 1.72 mmol) at 25° C. The mixture was stirred at 25° C. for 10 min. Then, 5-(tert-butyl)-1,2,4-oxadiazole-3-carboxylic acid (117 mg, 689 μmol) and HATU (263 mg, 689 μmol) were added. The mixture was stirred at 25° C. for 1 h. The reaction mixture was concentrated to give crude, which was purified by silica gel chromatography (petroleum ether/ethyl acetate=4/1) to give 5-(tert-butyl)-N-methyl-N-(2-methyl-4-(4,4,5,5-tetramethyl-1,3,2-dioxaborolan-2-yl)benzyl)-1,2,4-oxadiazole-3-carboxamide as a yellow oil (160 mg, 67% yield). LCMS: m/z=M+H⁺: 414.3. 6. Synthesis of tert-butyl 4-(4-(4-((5-(tert-butyl)-N-methyl-1,2,4-oxadiazole-3-carboxamido)methyl)-3-methylphenyl)pyridin-3-yl)piperazine-1-carboxylate

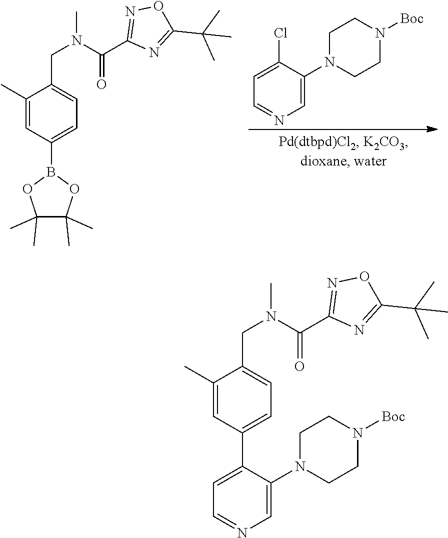 [SMILES: Cc1cc(B2OC(C)(C)C(C)(C)O2)ccc1CN(C)C(=O)c1noc(C(C)(C)C)n1 ; CC(C)(C)OC(=O)N1CCN(c2cnccc2Cl)CC1 ; Cc1cc(-c2ccncc2N2CCN(C(=O)OC(C)(C)C)CC2)ccc1CN(C)C(=O)c1noc(C(C)(C)C)n1]

A mixture of 5-(tert-butyl)-N-methyl-N-(2-methyl-4-(4,4,5,5-tetramethyl-1,3,2-dioxaborolan-2-yl)benzyl)-1,2,4-oxadiazole-3-carboxamide (140 mg, 339 μmol), tert-butyl 4-(4-chloropyridin-3-yl)piperazine-1-carboxylate (121 mg, 406 μmol), K₂CO₃ (140 mg, 1.02 mmol) and Pd(dtbpf)₂Cl₂ (22 mg, 34 μmol) in dioxane (3 mL) and water (0.6 mL) was bubbled with N₂ for 3 min. The mixture was then stirred at 90° C. for 24 h. The reaction mixture was concentrated to get crude material, which was purified by silica gel chromatography (1:1 petroleum ether/ethyl acetate) to give tert-butyl 4-(4-(4-((5-(tert-butyl)-N-methyl-1,2,4-oxadiazole-3-carboxamido)methyl)-3-methylphenyl)pyridin-3-yl)piperazine-1-carboxylate as a yellow oil (80 mg, 43% yield). LCMS: m/z=M+H⁺: 549.3.

### 7. Synthesis of 5-(tert-butyl)-N-methyl-N-(2-methyl-4-(3-(piperazin-1-yl)pyridin-4-yl)benzyl)-1,2,4-oxadiazole-3-carboxamide hydrochloride

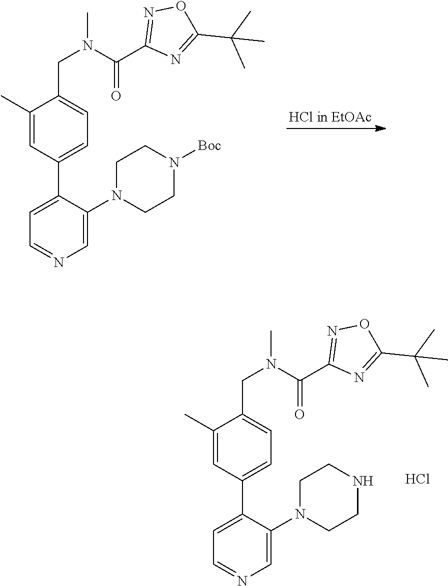 [SMILES: Cc1cc(-c2ccncc2N2CCN(C(=O)OC(C)(C)C)CC2)ccc1CN(C)C(=O)c1noc(C(C)(C)C)n1 ; Cc1cc(-c2ccncc2N2CCNCC2)ccc1CN(C)C(=O)c1noc(C(C)(C)C)n1 ; Cl]

To a solution of tert-butyl 4-(4-(4-((5-(tert-butyl)-N-methyl-1,2,4-oxadiazole-3-carboxamido)methyl)-3-methylphenyl)pyridin-3-yl)piperazine-1-carboxylate (80 mg, 146 μmol) in DCM (10 mL) was added an HCl solution in ethyl acetate (4 M, 1.5 mL). The reaction mixture was stirred at 25° C. for 30 minutes. The reaction mixture was concentrated to give crude 5-(tert-butyl)-N-methyl-N-(2-methyl-4-(3-(piperazin-1-yl)pyridin-4-yl)benzyl)-1,2,4-oxadiazole-3-carboxamide hydrochloride (60 mg, crude), which was carried forward without further purification. LCMS: m/z=M+H⁺: 449.2.

### 8. Synthesis of (E)-5-(tert-butyl)-N-(4-(3-(4-(4-(dimethylamino)but-2-enoyl)piperazin-1-yl)pyridin-4-yl)-2-methylbenzyl)-N-methyl-1, 2,4-oxadiazole-3-carboxamide

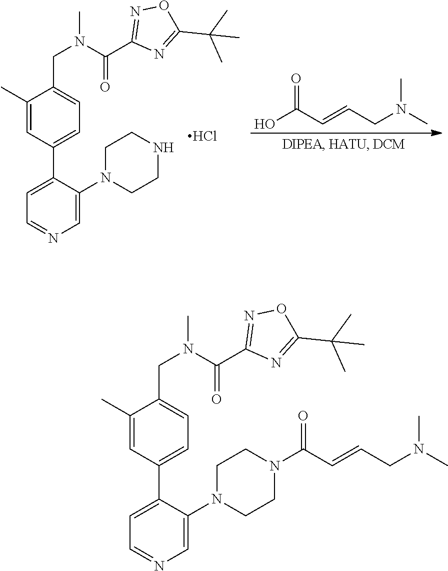

To a solution of 5-(tert-butyl)-N-methyl-N-(2-methyl-4-(3-(piperazin-1-yl)pyridin-4-yl)benzyl)-1,2,4-oxadiazole-3-carboxamide hydrochloride (60 mg, 134 μmol) in DCM (12 mL) was added DIPEA (52 mg, 401 μmol) at 25° C. The mixture was stirred at 25° C. for 10 min. Then, (E)-4-(dimethylamino)but-2-enoic acid (21 mg, 161 μmol) and HATU (61 mg, 161 μmol) were added. The mixture was stirred at 25° C. for 1 h. The reaction mixture was concentrated to give crude material, which was purified by prep-HPLC (Column: Boston Prime C18 150×30 mm×5 m; Condition: water (0.05% NH₃H₂O+10 mM NH₄HCO₃)-ACN; Begin B 47, End B 77, Gradient Time (min) 10, 100% B Hold Time (min) 2, Flow Rate (mL/min) 25, Injections 3.) to give (E)-5-(tert-butyl)-N-(4-(3-(4-(4-(dimethylamino)but-2-enoyl)piperazin-1-yl)pyridin-4-yl)-2-methylbenzyl)-N-methyl-1,2,4-oxadiazole-3-carboxamide as a white solid (30.2 mg, 40% yield). LCMS: m/z=M+H⁺: 560.5. ¹H NMR: (500 MHz, METHANOL-d₄) δ=8.32-8.24 (m, 2H), 7.66-7.56 (m, 2H), 7.42-7.29 (m, 2H), 6.82-6.72 (m, 1H), 6.63-6.60 (m, 1H), 4.89 (s, 2H), 3.61 (s, 4H), 3.16 (d, J=6.5 Hz, 2H), 3.14-3.08 (m, 3H), 3.00-2.89 (m, 4H), 2.47-2.34 (m, 3H), 2.31-2.28 (m, 6H), 1.54-1.45 (m, 9H).

### Example 72: (E)-1-(tert-butyl)-5-(4-(3-(4-(4-(dimethylamino)but-2-enoyl)piperazin-1-yl)pyridin-4-yl)-2-methylbenzyl)-1,5,6,7-tetrahydro-4H-pyrazolo[4,3-c]pyridin-4-one

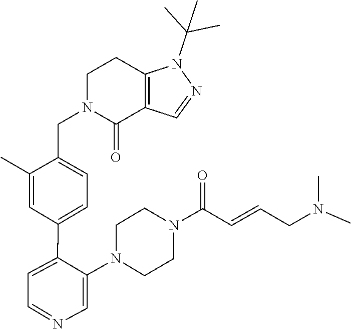

### 1. Synthesis of 5-(4-bromo-2-methylbenzyl)-1-(tert-butyl)-1,5,6,7-tetrahydro-4H-pyrazolo[4,3-c]pyridin-4-one

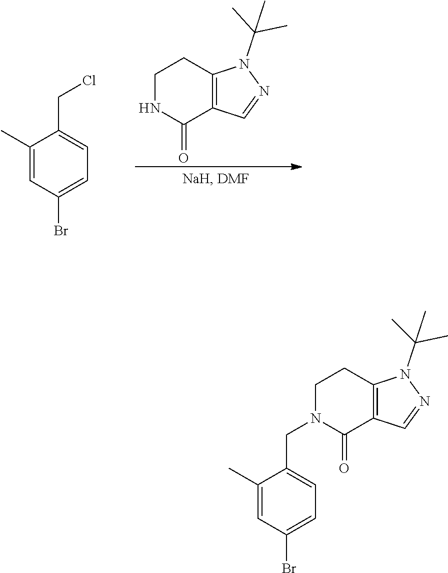

To a solution of 1-(tert-butyl)-1,5,6,7-tetrahydro-4H-pyrazolo[4,3-c]pyridin-4-one (440 mg, 2.28 mmol) in THF (20 mL) was added sodium hydride (455 mg, 11.4 mmol, 60% purity) at 0° C. The mixture was stirred at 20° C. for 30 minutes. Then 4-bromo-1-(chloromethyl)-2-methylbenzene (500 mg, 2.28 mmol) was added, and the mixture was stirred at 20° C. for 10 hours. The mixture was quenched with MeOH (2 mL) and concentrated under vacuum to give crude residue. The residue was purified by prep-TLC (ethyl acetate) to give 5-(4-bromo-2-methylbenzyl)-1-(tert-butyl)-1,5,6,7-tetrahydro-4H-pyrazolo[4,3-c]pyridin-4-one as a white solid (170 mg, 20% yield). LCMS: m/z=M+H⁺: 375.9, 377.9.

### 2. Synthesis of 1-(tert-butyl)-5-(2-methyl-4-(4,4,5,5-tetramethyl-1,3,2-dioxaborolan-2-yl)benzyl)-1,5,6,7-tetrahydro-4H-pyrazolo[4,3-c]pyridin-4-one

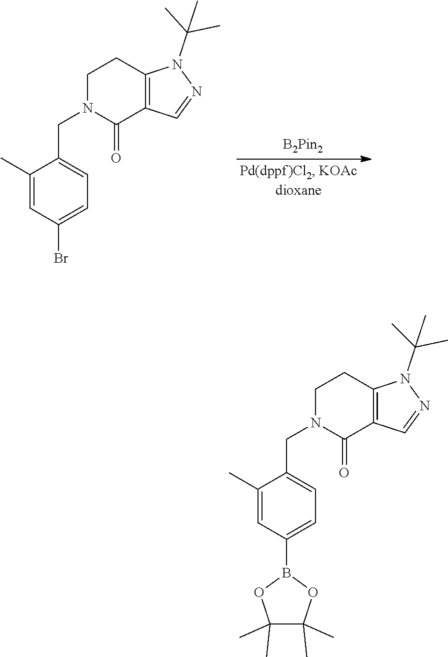 [SMILES: Cc1cc(Br)ccc1CN1CCc2c(cnn2C(C)(C)C)C1=O ; Cc1cc(B2OC(C)(C)C(C)(C)O2)ccc1CN1CCc2c(cnn2C(C)(C)C)C1=O]

To a solution of 5-(4-bromo-2-methylbenzyl)-1-(tert-butyl)-1,5,6,7-tetrahydro-4H-pyrazolo[4,3-c]pyridin-4-one (170 mg, 452 μmol) in dioxane (10 mL) was added B2Pin2 (229 mg, 904 μmol), KOAc (89 mg, 904 μmol) and Pd(dppf)Cl₂ (33 mg, 45 μmol) at 20° C. The mixture was stirred at 90° C. under N₂ for 16 hours. The mixture was concentrated under vacuum to give crude material, which was purified by silica gel chromatography (grading from petroleum ether to ethyl acetate) to give 1-(tert-butyl)-5-(2-methyl-4-(4,4,5,5-tetramethyl-1,3,2-dioxaborolan-2-yl)benzyl)-1,5,6,7-tetrahydro-4H-pyrazolo[4,3-c]pyridin-4-one as a colorless oil (140 mg, 73% yield). LCMS: m/z=M+H⁺: 424.3. 3. Synthesis of tert-butyl 4-(4-(4-((1-(tert-butyl)-4-oxo-1,4,6,7-tetrahydro-5H-pyrazolo[4,3-c]pyridin-5-yl)methyl)-3-methylphenyl)pyridin-3-yl)piperazine-1-carboxylate

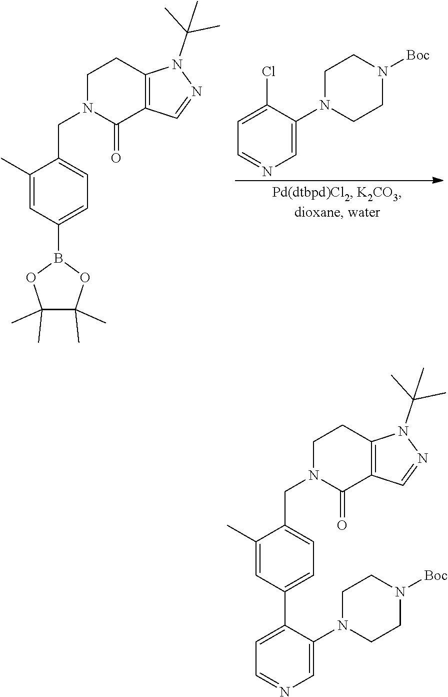 [SMILES: Cc1cc(B2OC(C)(C)C(C)(C)O2)ccc1CN1CCc2c(cnn2C(C)(C)C)C1=O ; CC(C)(C)OC(=O)N1CCN(c2cnccc2Cl)CC1 ; Cc1cc(-c2ccncc2N2CCN(C(=O)OC(C)(C)C)CC2)ccc1CN1CCc2c(cnn2C(C)(C)C)C1=O]

To a solution of 1-(tert-butyl)-5-(2-methyl-4-(4,4,5,5-tetramethyl-1,3,2-dioxaborolan-2-yl)benzyl)-1,5,6,7-tetrahydro-4H-pyrazolo[4,3-c]pyridin-4-one (140 mg, 331 μmol) in dioxane (5 mL) and water (1 mL) was added K₂CO₃ (91 mg, 661 μmol), tert-butyl 4-(4-chloropyridin-3-yl)piperazine-1-carboxylate (98 mg, 331 μmol) and Pd(dtbpf)Cl₂ (22 mg, 33 μmol) at 20° C. The mixture was stirred at 90° C. under N₂ for 2 hours. The mixture was concentrated under vacuum to give crude residue. The residue was purified by silica gel chromatography (grading from petroleum ether to ethyl acetate) to give tert-butyl 4-(4-(4-((1-(tert-butyl)-4-oxo-1,4,6,7-tetrahydro-5H-pyrazolo[4,3-c]pyridin-5-yl)methyl)-3-methylphenyl)pyridin-3-yl)piperazine-1-carboxylate as a yellow oil (100 mg, 54% yield). LCMS: m/z M+H⁺: 559.5. 4. Synthesis of 1-(tert-butyl)-5-(2-methyl-4-(3-(piperazin-1-yl)pyridin-4-yl)benzyl)-1,5,6,7-tetrahydro-4H-pyrazolo[4,3-c]pyridin-4-one hydrochloride

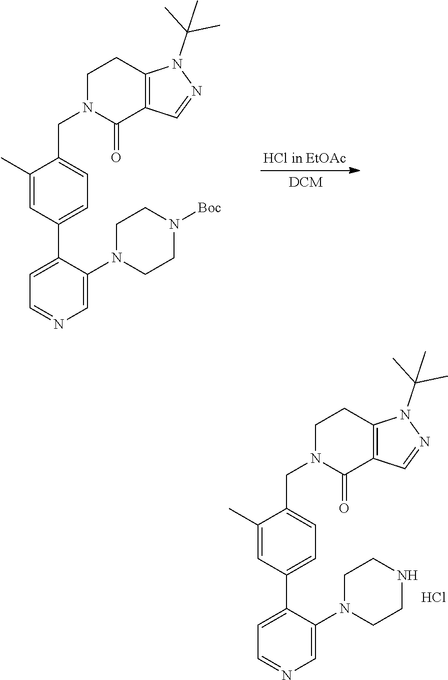 [SMILES: Cc1cc(-c2ccncc2N2CCN(C(=O)OC(C)(C)C)CC2)ccc1CN1CCc2c(cnn2C(C)(C)C)C1=O ; Cc1cc(-c2ccncc2N2CCNCC2)ccc1CN1CCc2c(cnn2C(C)(C)C)C1=O ; Cl]

To a solution of tert-butyl 4-(4-(4-((1-(tert-butyl)-4-oxo-1,4,6,7-tetrahydro-5H-pyrazolo[4,3-c]pyridin-5-yl)methyl)-3-methylphenyl)pyridin-3-yl)piperazine-1-carboxylate (100 mg, 179 μmol) in DCM (10 mL) was added an HCl solution in ethyl acetate (8 mL, 4 M) at 20° C. The mixture was stirred at 20° C. for 1 hour. The mixture was concentrated under vacuum to give 1-(tert-butyl)-5-(2-methyl-4-(3-(piperazin-1-yl)pyridin-4-yl)benzyl)-1,5,6,7-tetrahydro-4H-pyrazolo[4,3-c]pyridin-4-one hydrochloride as a yellow solid (80 mg, crude), which was carried forward without further purification. LCMS: m/z=M+H⁺: 459.3. 5. Synthesis of (E)-1-(tert-butyl)-5-(4-(3-(4-(4-(dimethylamino)but-2-enoyl)piperazin-1-yl)pyridin-4-yl)-2-methylbenzyl)-1,5,6,7-tetrahydro-4H-pyrazolo[4,3-c]pyridin-4-one

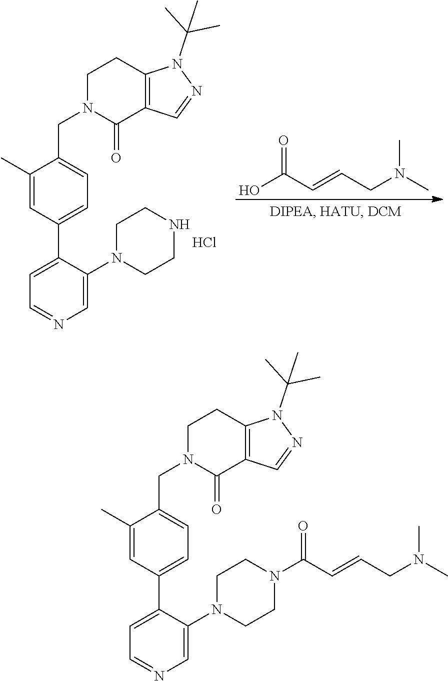

To a solution of 1-(tert-butyl)-5-(2-methyl-4-(3-(piperazin-1-yl)pyridin-4-yl)benzyl)-1,5,6,7-tetrahydro-4H-pyrazolo[4,3-c]pyridin-4-one hydrochloride (80 mg, 162 μmol) in DCM (50 mL) was added DIPEA (42 mg, 323 μmol), (E)-4-(dimethylamino)but-2-enoic acid (21 mg, 162 μmol) and HATU (62 mg, 162 μmol) at 20° C. The mixture was stirred at 20° C. for 30 minutes.

The mixture was concentrated under vacuum. The residue was purified by prep-HPLC (Column: Welch Xtimate C18 150×25 mm×5 μm; Condition: water (10 mM NH₄HCO₃)-ACN, Begin B 23 End B 53, Gradient Time (min) 10, 100% B Hold Time (min) 2, Flow Rate (mL/min): 25) to give (E)-1-(tert-butyl)-5-(4-(3-(4-(4-(dimethylamino)but-2-enoyl)piperazin-1-yl)pyridin-4-yl)-2-methylbenzyl)-1,5,6,7-tetrahydro-4H-pyrazolo[4,3-c]pyridin-4-one as a yellow solid (30 mg, 32% yield). LCMS: m/z=M+H⁺: 570.3. ¹H NMR: (500 MHz, DMSO-d₆) δ=8.30-8.27 (m, 2H), 7.71 (s, 1H), 7.58-7.55 (m, 2H), 7.26-7.22 (m, 2H), 6.59-6.55 (m, 2H), 4.65 (s, 2H), 3.51-3.47 (m, 6H), 3.21 (t, J=6.5 Hz, 2H), 2.99 (d, J=4.5 Hz, 2H), 2.85-2.81 (m, 4H), 2.33 (s, 3H), 2.12 (s, 6H), 1.57 (s, 9H).

### Example 73: (E)-N-(4-(3-(4-(4-(dimethylamino)but-2-enoyl)piperazin-1-yl)pyridin-4-yl)-2-methylbenzyl)-3-(1-methylcyclopropyl)-1,2,4-oxadiazole-5-carboxamide

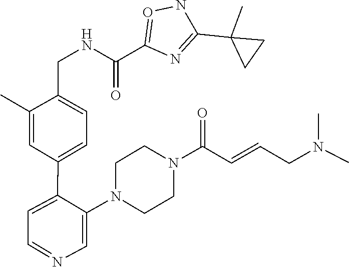

### 1. Synthesis of N-(2-methyl-4-(4,4,5,5-tetramethyl-1,3,2-dioxaborolan-2-yl)benzyl)-3-(1-methylcyclopropyl)-1,2,4-oxadiazole-5-carboxamide

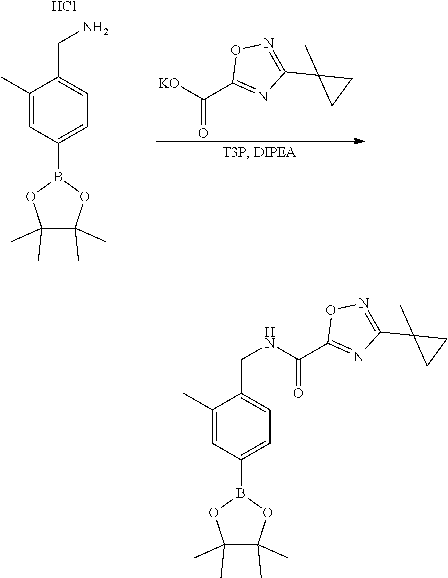

To a suspension of (2-methyl-4-(4,4,5,5-tetramethyl-1,3,2-dioxaborolan-2-yl)phenyl)methanamine hydrochloride (3.12 g, 12.6 mmol) and potassium 3-(1-methylcyclopropyl)-1,2,4-oxadiazole-5-carboxylate (2 g, 9.70 mmol) in EtOAc (30 mL) was added T3P (12.3 g, 19.4 mmol, 50% purity). Then DIPEA (6.27 g, 48.5 mmol, 8.5 mL) was added at 25° C. The mixture was stirred at 25° C. for 16 hours. The mixture was poured into water (100 mL) and extracted with EtOAc (2×80 mL). The organic layers were washed with brine (130 mL), dried over Na₂SO₄, filtered, and concentrated to give crude product. Crude material was purified by column chromatography on silica gel (petroleum ether/ethyl acetate=10/1) to give N-(2-methyl-4-(4,4,5,5-tetramethyl-1,3,2-dioxaborolan-2-yl)benzyl)-3-(1-methylcyclopropyl)-1,2,4-oxadiazole-5-carboxamide as a colorless oil (3.2 g, 83% yield). LCMS: m/z=M+H⁺: 398.3. ¹HNMR: (400 MHz, MeOH-d₄) δ: 7.52-7.57 (m, 2H), 7.27 (d, J=7.6 Hz, 1H), 4.58 (s, 2H), 2.37 (s, 3H), 1.52 (s, 3H), 1.34 (s, 12H), 1.27-1.30 (m, 2H), 0.93-0.96 (m, 2H).

### 2. Synthesis of tert-butyl 4-(4-(3-methyl-4-((3-(1-methylcyclopropyl)-1,2,4-oxadiazole-5-carboxamido)methyl)phenyl)pyridin-3-yl)piperazine-1-carboxylate

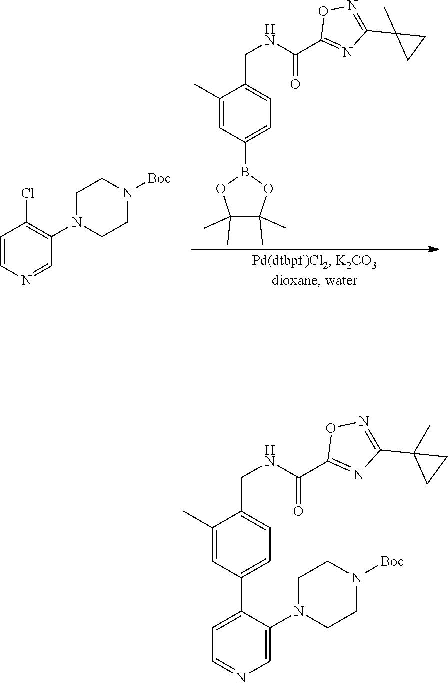 [SMILES: CC(C)(C)OC(=O)N1CCN(c2cnccc2Cl)CC1 ; Cc1cc(B2OC(C)(C)C(C)(C)O2)ccc1CNC(=O)c1nc(C2(C)CC2)no1 ; Cc1cc(-c2ccncc2N2CCN(C(=O)OC(C)(C)C)CC2)ccc1CNC(=O)c1nc(C2(C)CC2)no1]

3. Synthesis of tert-butyl 4-(4-(3-methyl-4-((3-(]-methylcyclopropyl)-1,2,4-oxadiazole-5-carboxamido)methyl)phenyl)pyridin-3-yl)piperazine-1-carboxylate was similar to that of tert-butyl 4-(4-(4-((5-(tert-butyl)-1,2,4-oxadiazole-3-carboxamido)methyl)-3-methylphenyl)pyridin-3-yl)piperazine-1-carboxylate in Example 57, Step 1. The crude material was purified by chromatography column on silica gel (petroleum ether/ethyl acetate=1/0 to 1/1) to give tert-butyl 4-(4-(3-methyl-4-((3-(1-methylcyclopropyl)-1,2,4-oxadiazole-5-carboxamido)methyl)phenyl)pyridin-3-yl)piperazine-1-carboxylate as a brown oil. (330 mg, 62% yield). LCMS: m/z=M+H⁺: 533.3. 4. Synthesis of N-(2-methyl-4-(3-(piperazin-1-yl)pyridin-4-yl)benzyl)-3-(1-methylcyclopropyl)-1,2,4-oxadiazole-5-carboxamide hydrochloride

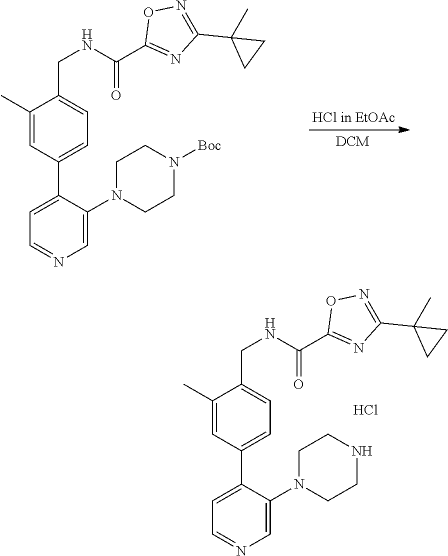 [SMILES: Cc1cc(-c2ccncc2N2CCN(C(=O)OC(C)(C)C)CC2)ccc1CNC(=O)c1nc(C2(C)CC2)no1 ; Cc1cc(-c2ccncc2N2CCNCC2)ccc1CNC(=O)c1nc(C2(C)CC2)no1 ; Cl]

5. Synthesis of N-(2-methyl-4-(3-(piperazin-1-yl)pyridin-4-yl)benzyl)-3-(1-methylcyclopropyl)-1,2,4-oxadiazole-5-carboxamide hydrochloride was similar to that of 5-(tert-butyl)-N-(4-(3-cyano-5-(piperazin-1-yl)pyridin-4-yl)-2-methylbenzyl)-1,2,4-oxadiazole-3-carboxamide hydrochloride in Example 69, Step 3. The crude material was dried by lyophilization to give N-(2-methyl-4-(3-(piperazin-1-yl)pyridin-4-yl)benzyl)-3-(1-methylcyclopropyl)-1,2,4-oxadiazole-5-carboxamide hydrochloride as a brown solid (220 mg, crude) which carried forward without further purification. LCMS: m/z=M+H⁺: 433.2. 6. Synthesis of(E)-N-(4-(3-(4-(4-(dimethylamino)but-2-enoyl)piperazin-1-yl)pyridin-4-yl)-2-methylbenzyl)-3-(1-methylcyclopropyl)-1,2,4-oxadiazole-5-carboxamide

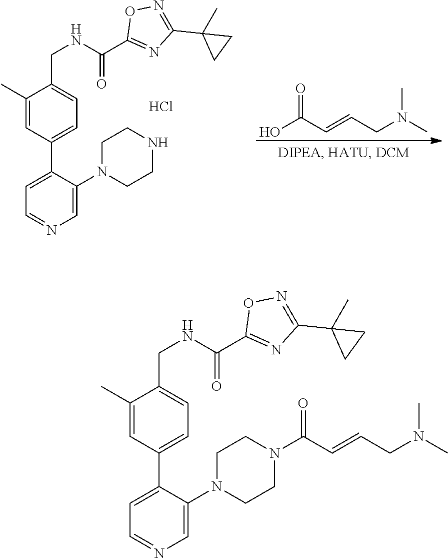

7. Synthesis of (E)-N-(4-(3-(4-(4-(dimethylamino)but-2-enoyl)piperazin-1-yl)pyridin-4-yl)-2-methylbenzyl)-3-(1-methylcyclopropyl)-1,2,4-oxadiazole-5-carboxamide was similar to that of 5-(tert-butyl)-N-(2-methyl-4-(3-(4-(oxirane-2-carbonyl)piperazin-1-yl)pyridin-4-yl)benzyl)-1,2,4-oxadiazole-3-carboxamide in Example 59. The crude material was purified by Prep-HPLC (Column: Agela DuraShell C18 150×25 mm×5 μm; Condition: water (0.05% NH₃H₂O+10 mM NH₄HCO₃)—CAN, Begin B 39, End B 59, Gradient Time (min) 10, 100% B Hold Time (min) 2, Flow Rate (ml/min) 25.) to give (E)-N-(4-(3-(4-(4-(dimethylamino)but-2-enoyl)piperazin-1-yl)pyridin-4-yl)-2-methylbenzyl)-3-(1-methylcyclopropyl)-1,2,4-oxadiazole-5-carboxamide as a white solid (81 mg, 38% yield). LCMS: m/z=M+H⁺: 544.5. ¹H NMR: (500 MHz, METHANOL-d₄) δ=8.36-8.30 (m, 2H), 7.56 (d, J=6.5 Hz, 2H), 7.42 (d, J=8.5 Hz, 1H), 7.29 (d, J=4.5 Hz, 1H), 6.80-6.70 (m, 1H), 6.59 (d, J=5.5 Hz, 1H), 4.62-4.56 (m, 2H), 3.60 (s, 4H), 3.14 (d, J=6.5 Hz, 2H), 2.98-2.93 (m, 4H), 2.45 (s, 3H), 2.28 (s, 6H), 1.53 (s, 3H), 1.32-1.26 (m, 2H), 0.98-0.94 (m, 2H).

### Example 74: (E)-5-(tert-butyl)-N-(4-(3-(4-(4-(dimethylamino)but-2-enoyl)-3-methylpiperazin-1-yl)pyridin-4-yl)-2-methylbenzyl)-1,2,4-oxadiazole-3-carboxamide

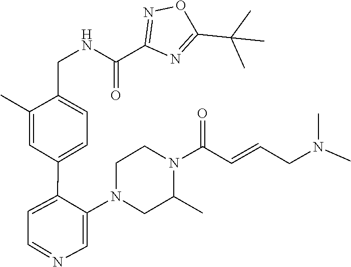

### 1. Synthesis of tert-butyl-4-(4-chloropyridin-3-yl)-2-methylpiperazine-1-carboxylate

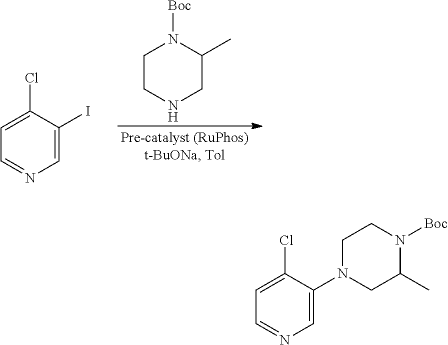 [SMILES: Clc1ccncc1I ; CC1CNCCN1C(=O)OC(C)(C)C ; CC1CN(c2cnccc2Cl)CCN1C(=O)OC(C)(C)C]

A mixture of 4-chloro-3-iodopyridine (2 g, 8.35 mmol), tert-butyl-2-methylpiperazine-1-carboxylate (1.67 g, 8.35 mmol), RuPhos Pd G3 (699 mg, 0.84 mmol) and sodium tert-butoxide (2.41 g, 25.1 mmol) in toluene (60 mL) was bubbled with N₂ for 2 min. Then the mixture was stirred at 90° C. under N₂ for 7 hours. The solvent was removed under vacuum to give crude material which was purified by silica gel column chromatography (grading from 0% to 50% ethyl acetate in petroleum ether) to give tert-butyl-4-(4-chloropyridin-3-yl)-2-methylpiperazine-1-carboxylate as a pale yellow oil (1.1 g, 42% yield). ¹H NMR: (500 MHz, METHANOL-d₄) δ=8.31 (s, 1H), 8.19 (d, J=5.0 Hz, 1H), 7.50 (d, J=5.0 Hz, 1H), 4.40-4.33 (m, 1H), 4.02-3.93 (m, 1H), 3.39-3.32 (m, 3H), 3.00-2.81 (m, 2H), 1.54-1.49 (m, 9H), 1.41 (d, J=7.0 Hz, 3H).

### 2. Synthesis of tert-butyl-4-(4-(4-((5-(tert-butyl)-1,2,4-oxadiazole-3-carboxamido)methyl)-3-methylphenyl)pyridin-3-yl)-2-methylpiperazine-1-carboxylate

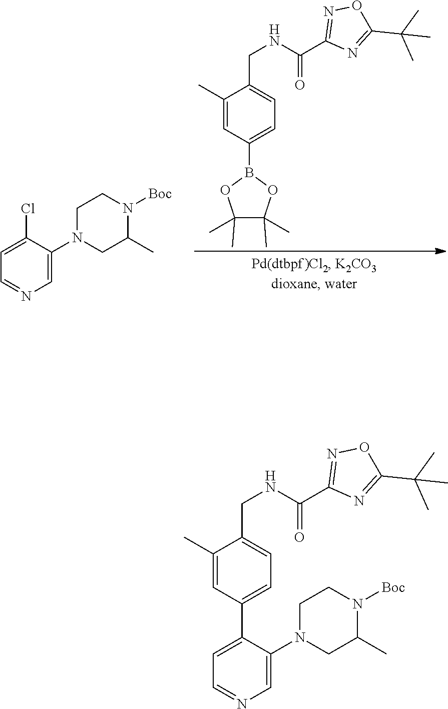 [SMILES: CC1CN(c2cnccc2Cl)CCN1C(=O)OC(C)(C)C ; Cc1cc(B2OC(C)(C)C(C)(C)O2)ccc1CNC(=O)c1noc(C(C)(C)C)n1 ; Cc1cc(-c2ccncc2N2CCN(C(=O)OC(C)(C)C)C(C)C2)ccc1CNC(=O)c1noc(C(C)(C)C)n1] [0820] 3. Synthesis of tert-butyl-4-(4-(4-((5-(tert-butyl)-1,2,4-oxadiazole-3-carboxamido)methyl)-3-methylphenyl)pyridin-3-yl)-2-methylpiperazine-1-carboxylate was similar to that of tert-butyl 4-(4-(4-((5-(tert-butyl)-1,2,4-oxadiazole-3-carboxamido)methyl)-3-methylphenyl)pyridin-3-yl)piperazine-1-carboxylate in Example 57, Step 1. The crude material was purified by silica gel column chromatography (grading from 0% to 100% ethyl acetate in petroleum ether) to give tert-butyl-4-(4-(4-((5-(tert-butyl)-1,2,4-oxadiazole-3-carboxamido)methyl)-3-methylphenyl)pyridin-3-yl)-2-methylpiperazine-1-carboxylate as a colorless oil (500 mg, 47% yield). ¹H NMR: (500 MHz, METHANOL-d₄) δ=8.30-8.26 (m, 2H), 7.52 (s, 1H), 7.47-7.42 (m, 2H), 7.27 (d, J=5.0 Hz, 1H), 4.66 (s, 2H), 4.21 (d, J=5.0 Hz, 1H), 3.71-3.65 (m, 1H), 3.05-2.99 (m, 2H), 2.97-2.86 (m, 2H), 2.60-2.52 (m, 1H), 2.47 (s, 3H), 1.52-1.49 (m, 9H), 1.45 (s, 9H), 1.08 (d, J=6.5 Hz, 3H).

### 4. Synthesis of 5-(tert-butyl)-N-(2-methyl-4-(3-(3-methylpiperazin-1-yl)pyridin-4-yl)benzyl)-1,2,4-oxadiazole-3-carboxamide hydrochloride

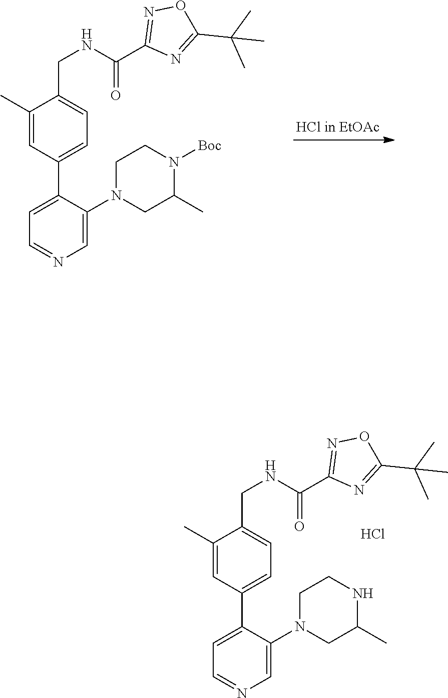 [SMILES: Cc1cc(-c2ccncc2N2CCN(C(=O)OC(C)(C)C)C(C)C2)ccc1CNC(=O)c1noc(C(C)(C)C)n1 ; Cc1cc(-c2ccncc2N2CCNC(C)C2)ccc1CNC(=O)c1noc(C(C)(C)C)n1 ; Cl] [0822] 5. Synthesis of 5-(tert-butyl)-N-(2-methyl-4-(3-(3-methylpiperazin-1-yl)pyridin-4-yl)benzyl)-1,2,4-oxadiazole-3-carboxamide hydrochloride was similar to that of 5-(tert-butyl)-N-(4-(3-cyano-5-(piperazin-1-yl)pyridin-4-yl)-2-methylbenzyl)-1,2,4-oxadiazole-3-carboxamide hydrochloride in Example 69, Step 3. The crude material was isolated as a yellow oil (400 mg, crude) and was carried forward without further purification. LCMS: m/z=M+H⁺: 449.3. 6. Synthesis of(E)-5-(tert-butyl)-N-(4-(3-(4-(4-(dimethylamino)but-2-enoyl)-3-methylpiperazin-1-yl)pyridin-4-yl)-2-methylbenzyl)-1,2,4-oxadiazole-3-carboxamide

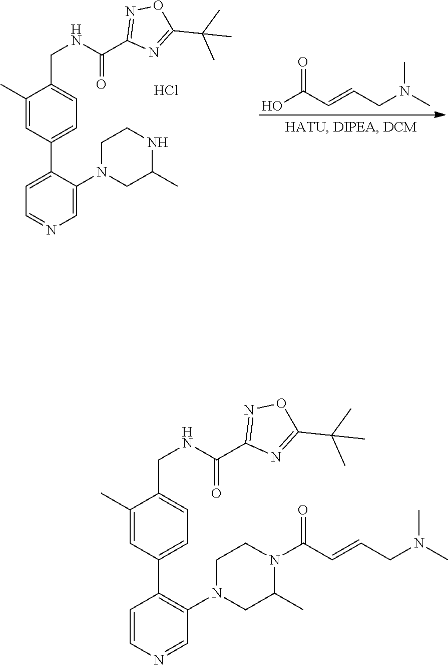

7. Synthesis of (E)-5-(tert-butyl)-N-(4-(3-(4-(4-(dimethylamino)but-2-enoyl)-3-methylpiperazin-1-yl)pyridin-4-yl)-2-methylbenzyl)-1,2,4-oxadiazole-3-carboxamide was similar to that of 5-(tert-butyl)-N-(2-methyl-4-(3-(4-(oxirane-2-carbonyl)piperazin-1-yl)pyridin-4-yl)benzyl)-1,2,4-oxadiazole-3-carboxamide in Example 59. The crude material was purified by prep HPLC (Column: Boston Prime C18 150×30 mm×5 μm; Condition: water (0.05% NH₃H₂O+10 mM NH₄HCO₃)—ACN, Begin B 38, End B 53, Gradient Time (min) 14, 100% B Hold Time (min) 2, Flow Rate (mL/min) 25) to give (E)-5-(tert-butyl)-N-(4-(3-(4-(4-(dimethylamino)but-2-enoyl)-3-methylpiperazin-1-yl)pyridin-4-yl)-2-methylbenzyl)-1,2,4-oxadiazole-3-carboxamide as a white solid (114 mg, 36% yield). LCMS: m/z=M+H⁺: 560.5. ¹H NMR: (500 MHz, METHANOL-d₄) δ=8.32-8.26 (m, 2H), 7.53 (s, 1H), 7.49-7.43 (m, 2H), 7.28 (d, J=5.0 Hz, 1H), 6.75 (s, 1H), 6.58 (s, 1H), 4.70-4.66 (m, 3H), 4.36-4.18 (m, 1H), 3.84-3.21 (m, 1H), 3.19-3.06 (m, 4H), 2.96-2.83 (m, 1H), 2.63-2.60 (m, 1H), 2.47 (s, 3H), 2.28 (s, 6H), 1.55-1.47 (m, 9H), 1.20-1.10 (m, 3H).

### Example 75: (E)-5-(tert-butyl)-N-(4-(3-(4-(4-(dimethylamino)but-2-enoyl)-3-(trifluoromethyl)piperazin-1-yl)pyridin-4-yl)-2-methylbenzyl)-1,2,4-oxadiazole-3-carboxamide

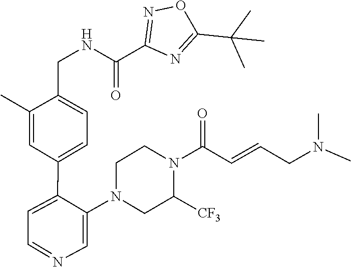

### 1. Synthesis of tert-butyl-4-(4-chloropyridin-3-yl)-2-(trifluoromethyl)piperazine-]-carboxylate

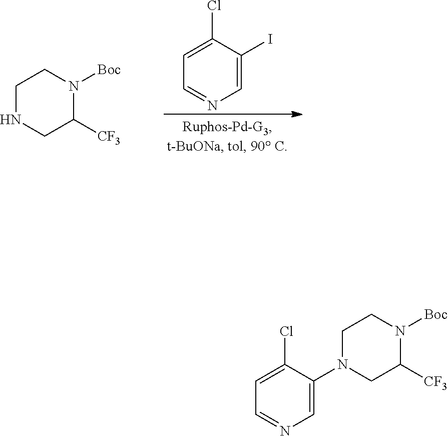 [SMILES: CC(C)(C)OC(=O)N1CCNCC1C(F)(F)F ; Clc1ccncc1I ; CC(C)(C)OC(=O)N1CCN(c2cnccc2Cl)CC1C(F)(F)F]

A mixture of 4-chloro-3-iodopyridine (466 mg, 1.95 mmol,), tert-butyl-2-(trifluoromethyl)piperazine-1-carboxylate (450 mg, 1.77 mmol), RuPhos Pd G3 (148 mg, 0.18 mmol) and sodium tert-butoxide (510 mg, 5.31 mmol) in toluene (15 mL) was bubbled with N₂ for 2 min. Then the mixture was stirred at 90° C. under N₂ for 8 hours. The solvent was removed under vacuum to give a crude residue. This material was purified by silica gel column chromatography (grading from 0%-30% ethyl acetate in petroleum ether) to give tert-butyl-4-(4-chloropyridin-3-yl)-2-(trifluoromethyl)piperazine-1-carboxylate as a yellow oil (330 mg, 51% yield). ¹H NMR: (400 MHz, METHANOL-d₄) δ=8.36-8.28 (m, 1H), 8.18 (d, J=5.2 Hz, 1H), 7.48 (d, J=5.2 Hz, 1H), 4.80-4.70 (m, 1H), 4.18-4.09 (m, 1H), 3.73 (d, J=13.2 Hz, 1H), 3.50-3.35 (m, 2H), 3.20-3.09 (m, 1H), 2.99-2.84 (m, 1H), 1.53-1.45 (m, 9H).

### 2. Synthesis of tert-butyl-4-(4-(4-((5-(tert-butyl)-1,2,4-oxadiazole-3-carboxamido)methyl)-3-methylphenyl)pyridin-3-yl)-2-(trifluoromethyl)piperazine-1-carboxylate

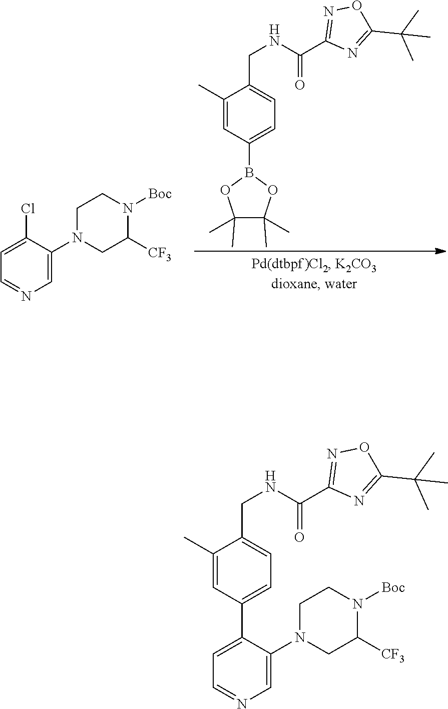 [SMILES: CC(C)(C)OC(=O)N1CCN(c2cnccc2Cl)CC1C(F)(F)F ; Cc1cc(B2OC(C)(C)C(C)(C)O2)ccc1CNC(=O)c1noc(C(C)(C)C)n1 ; Cc1cc(-c2ccncc2N2CCN(C(=O)OC(C)(C)C)C(C(F)(F)F)C2)ccc1CNC(=O)c1noc(C(C)(C)C)n1]

3. Synthesis of tert-butyl-4-(4-(4-((5-(tert-butyl)-1,2,4-oxadiazole-3-carboxamido)methyl)-3-methylphenyl)pyridin-3-yl)-2-(trifluoromethyl)piperazine-1-carboxylate was similar to that of tert-butyl 4-(4-(4-((5-(tert-butyl)-1,2,4-oxadiazole-3-carboxamido)methyl)-3-methylphenyl)pyridin-3-yl)piperazine-1-carboxylate in Example 57, Step 1. The crude material was purified by silica gel column chromatography (grading from 0% to 100% ethyl acetate in petroleum ether) to give tert-butyl-4-(4-(4-((5-(tert-butyl)-1,2,4-oxadiazole-3-carboxamido)methyl)-3-methylphenyl)pyridin-3-yl)-2-(trifluoromethyl)piperazine-1-carboxylate as a colorless oil (80 mg, 26% yield). LCMS: m/z=M+H⁺: 603.3. 4. Synthesis of 5-(tert-butyl)-N-(2-methyl-4-(3-(3-(trifluoromethyl)piperazin-1-yl)pyridin-4-yl)benzyl)-1,2,4-oxadiazole-3-carboxamide hydrochloride

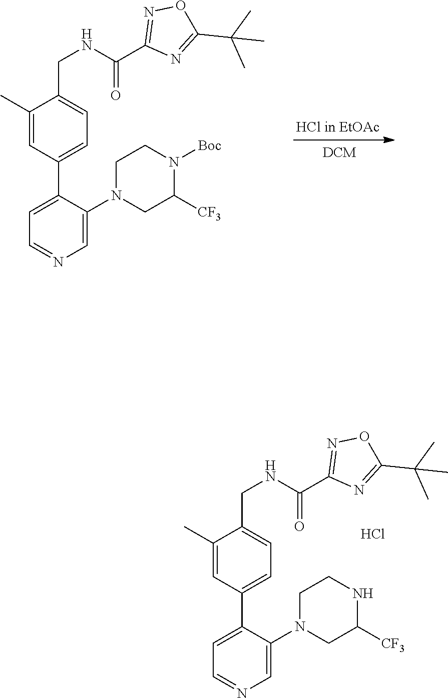 [SMILES: Cc1cc(-c2ccncc2N2CCN(C(=O)OC(C)(C)C)C(C(F)(F)F)C2)ccc1CNC(=O)c1noc(C(C)(C)C)n1 ; Cc1cc(-c2ccncc2N2CCNC(C(F)(F)F)C2)ccc1CNC(=O)c1noc(C(C)(C)C)n1 ; Cl]

5. Synthesis of 5-(tert-butyl)-N-(2-methyl-4-(3-(3-(trifluoromethyl)piperazin-1-yl)pyridin-4-yl)benzyl)-1,2,4-oxadiazole-3-carboxamide hydrochloride was similar to that of 5-(tert-butyl)-N-(4-(3-cyano-5-(piperazin-1-yl)pyridin-4-yl)-2-methylbenzyl)-1,2,4-oxadiazole-3-carboxamide hydrochloride in Example 69, Step 3. The crude material was purified by prep HPLC (Column: Boston Green ODS 150×30 mm×5 μm; Condition: water (0.05% HCl)—ACN, Begin B 20, End B 40, Gradient Time (min) 10, 100% B Hold Time (min) 2, Flow Rate (mL/min) 25) to give 5-(tert-butyl)-N-(2-methyl-4-(3-(3-(trifluoromethyl)piperazin-1-yl)pyridin-4-yl)benzyl)-1,2,4-oxadiazole-3-carboxamide hydrochloride as a pale yellow solid (45 mg, 67% yield). LCMS: m/z=M+H⁺: 503.3. 6. Synthesis of (E)-4-bromobut-2-enoyl chloride

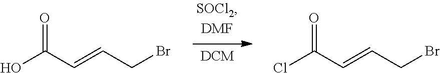 [SMILES: O=C(O)/C=C/CBr ; O=C(Cl)/C=C/CBr]

To the mixture of (E)-4-bromobut-2-enoic acid (200 mg, 1.21 mmol) in DCM (5 mL) was added dropwise SOCl₂ (288 mg, 2.42 mmol), followed by a drop of DMF (catalytic) at 25° C. The mixture was stirred at 25° C. for 2 hours. The reaction was concentrated directly to get a crude (E)-4-bromobut-2-enoyl chloride as a dark red gum (220 mg, crude) which was carried forward without further characterization or purification.

### 7. Synthesis of(E)-N-(4-(3-(4-(4-bromobut-2-enoyl)-3-(trifluoromethyl)piperazin-1-yl)pyridin-4-yl)-2-methylbenzyl)-5-(tert-butyl)-1,2,4-oxadiazole-3-carboxamide

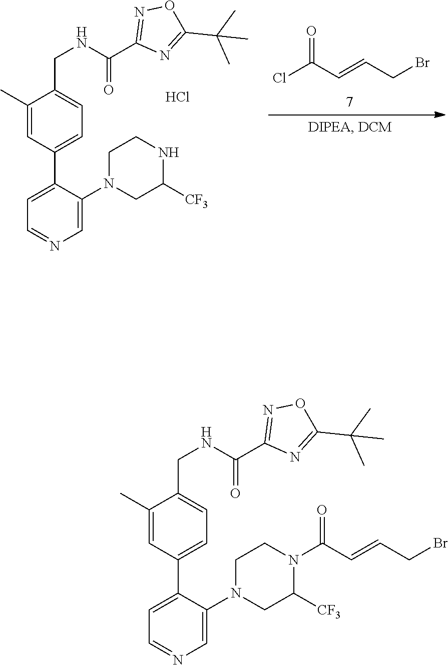 [SMILES: Cc1cc(-c2ccncc2N2CCNC(C(F)(F)F)C2)ccc1CNC(=O)c1noc(C(C)(C)C)n1 ; Cl ; O=C(Cl)/C=C/CBr ; Cc1cc(-c2ccncc2N2CCN(C(=O)/C=C/CBr)C(C(F)(F)F)C2)ccc1CNC(=O)c1noc(C(C)(C)C)n1]

To the mixture of 5-(tert-butyl)-N-(2-methyl-4-(3-(3-(trifluoromethyl)piperazin-1-yl)pyridin-4-yl)benzyl)-1,2,4-oxadiazole-3-carboxamide hydrochloride (45 mg, 0.089 mmol) and DIPEA (35 mg, 0.27 mmol) in DCM (5 mL) was added compound 7 (25 mg, 0.13 mmol) in DCM (0.5 mL) at 0° C. under N₂. The mixture was stirred at 25° C. for 1 hour. The reaction mixture was used to next step without workup. LCMS: m/z=M+H⁺: 649.2, 651.2. 8. Synthesis of (E)-5-(tert-butyl)-N-(4-(3-(4-(4-(dimethylamino)but-2-enoyl)-3-methylpiperazin-1-yl)pyridin-4-yl)-2-methylbenzyl)-1,2,4-oxadiazole-3-carboxamide

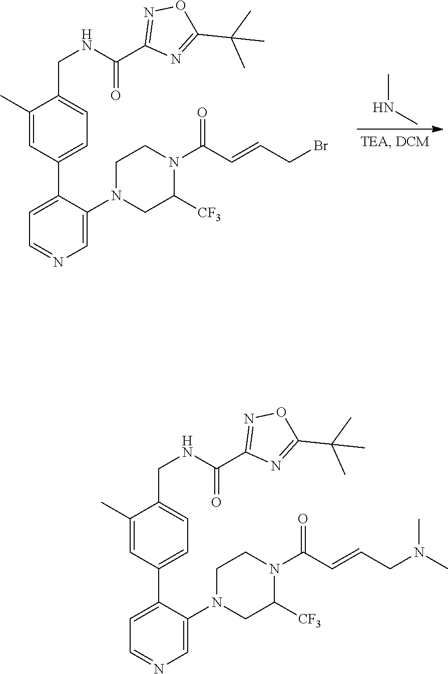

To the solution of (E)-N-(4-(3-(4-(4-bromobut-2-enoyl)-3-(trifluoromethyl)piperazin-1-yl)pyridin-4-yl)-2-methylbenzyl)-5-(tert-butyl)-1,2,4-oxadiazole-3-carboxamide (58 mg, 0.089 mmol) and DIPEA (35 mg, 0.27 mmol) in DCM (8 mL) was added MeNH₂ (2 M, 1.03 mL) at 25° C. under N₂. The mixture was stirred at 25° C. for 12 hours. The solvent was removed under vacuum to give a crude residue. This material was purified by prep HPLC (Column: Welch Xtimate C18 150×25 mm×5 μm; Condition: water (10 mM NH₄HCO₃)—ACN, Begin B 35, End B 55, Gradient Time (min) 15, 100% B Hold Time (min) 2, Flow Rate (mL/min) 25) to give (E)-5-(tert-butyl)-N-(4-(3-(4-(4-(dimethylamino)but-2-enoyl)-3-methylpiperazin-1-yl)pyridin-4-yl)-2-methylbenzyl)-1,2,4-oxadiazole-3-carboxamide as an off white solid (13 mg, 24% yield over 2 steps). LCMS: m/z=M+H⁺: 614.2. ¹H NMR: (500 MHz, METHANOL-d₄) δ=8.32 (s, 2H), 7.54 (s, 1H), 7.47-7.40 (m, 2H), 7.31 (d, J=5.0 Hz, 1H), 6.92-6.79 (m, 1H), 6.72-6.53 (m, 1H), 5.43-4.99 (m, 1H), 4.66 (s, 2H), 4.29-3.79 (m, 1H), 3.73 (d, J=12.5 Hz, 1H), 3.31-3.12 (m, 4H), 3.10-2.83 (m, 1H), 2.57-2.48 (m, 1H), 2.45 (s, 3H), 2.33-2.24 (m, 6H), 1.50 (s, 9H).

### Example 76: (E)-5-(tert-butyl)-N-(4-(3-(4-(4-(dimethylamino)but-2-enoyl)piperazin-1-yl)-5-fluoropyridin-4-yl)-2-methylbenzyl)-1,2,4-oxadiazole-3-carboxamide

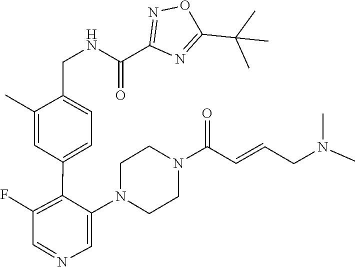 [SMILES: Cc1cc(-c2c(F)cncc2N2CCN(C(=O)/C=C/CN(C)C)CC2)ccc1CNC(=O)c1noc(C(C)(C)C)n1]

### 1. Synthesis of tert-butyl 4-(4-chloro-5-fluoropyridin-3-yl)piperazine-1-carboxylate

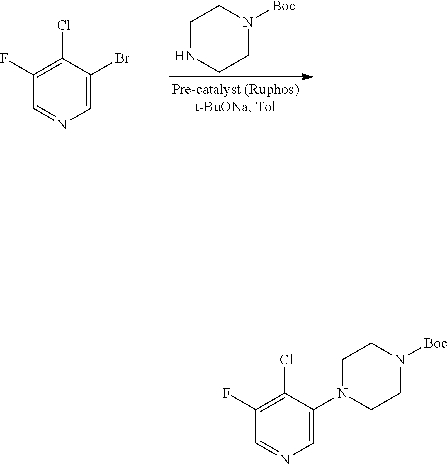 [SMILES: Fc1cncc(Br)c1Cl ; CC(C)(C)OC(=O)N1CCNCC1 ; CC(C)(C)OC(=O)N1CCN(c2cncc(F)c2Cl)CC1]

To a solution of 3-bromo-4-chloro-5-fluoropyridine (300 mg, 1.43 mmol) in toluene (3 mL) was added tert-butyl piperazine-1-carboxylate (252 mg, 1.35 mmol) and sodium tert-butoxide (274 mg, 2.85 mmol) at 15° C. Then Pre-catalyst (Ruphos) (179 mg, 214 μmol) was added at 15° C. The mixture was stirred at 80° C. for 30 min under N₂. The mixture was filtered and concentrated under vacuum to give crude material. The crude was purified by prep-TLC (petroleum ether/ethyl acetate=2/1) to give tert-butyl 4-(4-chloro-5-fluoropyridin-3-yl)piperazine-1-carboxylate as a yellow solid (110 mg, 19% yield). LCMS: m/z=M+H⁺: 316.3.

### 2. Synthesis of tert-butyl 4-(4-(4-((5-(tert-butyl)-1,2,4-oxadiazole-3-carboxamido)methyl)-3-methylphenyl)-5-fluoropyridin-3-yl)piperazine-1-carboxylate

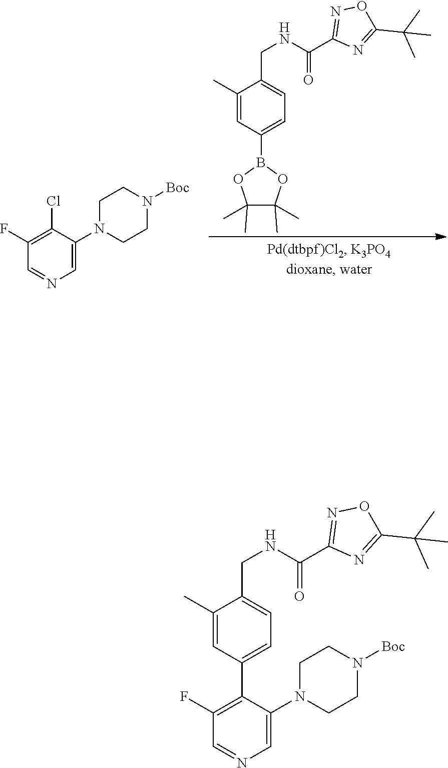 [SMILES: CC(C)(C)OC(=O)N1CCN(c2cncc(F)c2Cl)CC1 ; Cc1cc(B2OC(C)(C)C(C)(C)O2)ccc1CNC(=O)c1noc(C(C)(C)C)n1 ; Cc1cc(-c2c(F)cncc2N2CCN(C(=O)OC(C)(C)C)CC2)ccc1CNC(=O)c1noc(C(C)(C)C)n1]

To a solution of tert-butyl 4-(4-chloro-5-fluoropyridin-3-yl)piperazine-1-carboxylate (100 mg, 317 μmol) in dioxane (10 mL) and water (1 mL) was added 5-(tert-butyl)-N-(2-methyl-4-(4,4,5,5-tetramethyl-1,3,2-dioxaborolan-2-yl)benzyl)-1,2,4-oxadiazole-3-carboxamide(139 mg, 348 μmol) and K₃PO₄ (202 mg, 950 μmol) at 15° C. Then Pd(dtbpf)Cl₂ (40 mg, 47.5 μmol) was added at 15° C. The mixture was stirred at 15° C. for 3 hours under N₂. The mixture was filtered and the filtrate was concentrated under vacuum to give crude material. The crude product was purified by prep-TLC (petroleum ether/ethyl acetate=1/1) to give the tert-butyl 4-(4-(4-((5-(tert-butyl)-1,2,4-oxadiazole-3-carboxamido)methyl)-3-methylphenyl)-5-fluoropyridin-3-yl)piperazine-1-carboxylate as a yellow oil (120 mg, 48% yield). LCMS: m/z=M+H⁺: 553.7.

### 3. Synthesis of 5-(tert-butyl)-N-(4-(3-fluoro-5-(piperazin-1-yl)pyridin-4-yl)-2-methylbenzyl)-1,2,4-oxadiazole-3-carboxamide hydrochloride

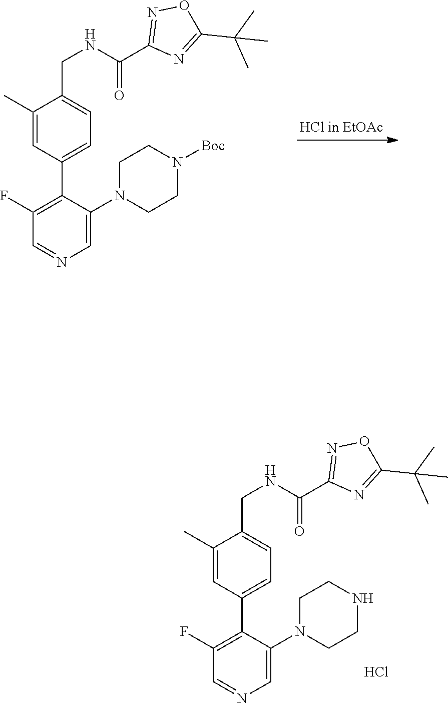 [SMILES: Cc1cc(-c2c(F)cncc2N2CCN(C(=O)OC(C)(C)C)CC2)ccc1CNC(=O)c1noc(C(C)(C)C)n1 ; Cc1cc(-c2c(F)cncc2N2CCNCC2)ccc1CNC(=O)c1noc(C(C)(C)C)n1 ; Cl]

A solution of tert-butyl 4-(4-(4-((5-(tert-butyl)-1,2,4-oxadiazole-3-carboxamido)methyl)-3-methylphenyl)-5-fluoropyridin-3-yl)piperazine-1-carboxylate (120 mg, 217 μmol) in DCM (10 mL) and an HCl solution in ethyl acetate (4 M, 10 mL) was stirred at 15° C. for 1 hour. The mixture was concentrated under vacuum to give 5-(tert-butyl)-N-(4-(3-fluoro-5-(piperazin-1-yl)pyridin-4-yl)-2-methylbenzyl)-1,2,4-oxadiazole-3-carboxamide hydrochloride as a yellow solid (85 mg, crude), which was carried forward without further purification. LCMS: m/z=M+H⁺: 453.3.

### 4. Synthesis of (E)-5-(tert-butyl)-N-(4-(3-(4-(4-(dimethylamino)but-2-enoyl)piperazin-1-yl)-5-fluoropyridin-4-yl)-2-methylbenzyl)-1,2,4-oxadiazole-3-carboxamide

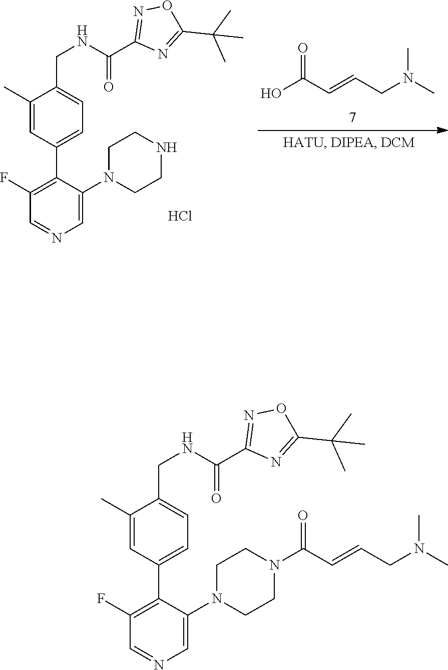 [0842] 5. Synthesis of (E)-5-(tert-butyl)-N-(4-(3-(4-(4-(dimethylamino)but-2-enoyl)piperazin-1-yl)-5-fluoropyridin-4-yl)-2-methylbenzyl)-1,2,4-oxadiazole-3-carboxamide was similar to that of 5-(tert-butyl)-N-(2-methyl-4-(3-(4-(oxirane-2-carbonyl)piperazin-1-yl)pyridin-4-yl)benzyl)-1,2,4-oxadiazole-3-carboxamide in Example 59. The crude material was purified by prep-HPLC (Column: Welch Xtimate C18 150×25 mm×5 μm; Condition: water (10 mM NH₄HCO₃)-ACN, Begin B 34, End B 64, Gradient Time (min) 10, 100% B Hold Time (min) 2, Flow Rate (mL/min) 25.) to give (E)-5-(tert-butyl)-N-(4-(3-(4-(4-(dimethylamino)but-2-enoyl)piperazin-1-yl)-5-fluoropyridin-4-yl)-2-methylbenzyl)-1,2,4-oxadiazole-3-carboxamide as a yellow solid (48 mg, 51% yield). LCMS: m/z=M+H⁺: 564.4. ¹H NMR: (500 MHz, DMSO-d₆) δ=9.47-9.44 (m, 1H), 8.33 (s, 1H), 8.21 (s, 1H), 7.41-7.35 (m, 3H), 6.62-6.52 (m, 2H), 4.51 (d, J=6.0 Hz, 2H), 3.41-3.39 (m, 4H), 2.99 (d, J=4.5 Hz, 2H), 2.84-2.82 (m, 4H), 2.38 (s, 3H), 2.15-2.08 (m, 6H), 1.46-1.41 (m, 9H).

### Example 77: (E)-N-(4-(3-(4-(4-(dimethylamino)but-2-enoyl)piperazin-1-yl)-5-fluoropyridin-4-yl)-2-methylbenzyl)-5-(1-methylcyclopropyl)-1,2,4-oxadiazole-3-carboxamide

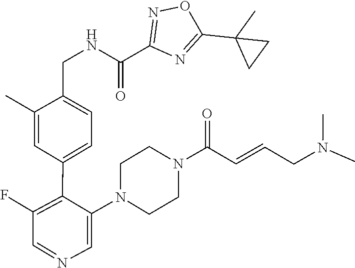 [SMILES: Cc1cc(-c2c(F)cncc2N2CCN(C(=O)/C=C/CN(C)C)CC2)ccc1CNC(=O)c1noc(C2(C)CC2)n1] [0844] 1. Synthesis of N-(2-methyl-4-(4,4,5,5-tetramethyl-1,3,2-dioxaborolan-2-yl)benzyl)-5-(1-methylcyclopropyl)-1,2,4-oxadiazole-3-carboxamide

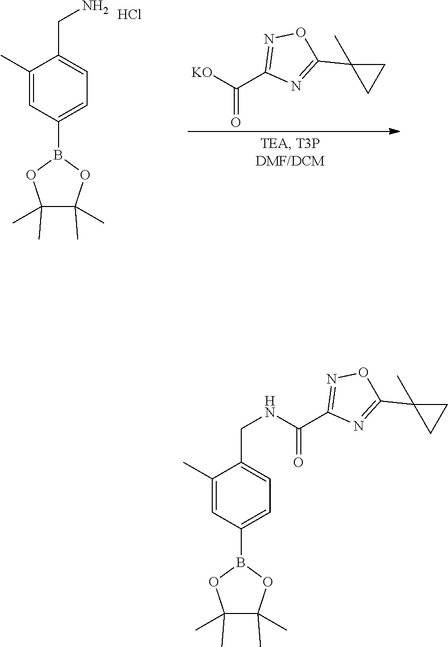

2. Synthesis of N-(2-methyl-4-(4,4,5,5-tetramethyl-1,3,2-dioxaborolan-2-yl)benzyl)-5-(1-methylcyclopropyl)-1,2,4-oxadiazole-3-carboxamide was similar to that of N-(2-methyl-4-(4,4,5,5-tetramethyl-1,3,2-dioxaborolan-2-yl)benzyl)-3-(1-methylcyclopropyl)-1,2,4-oxadiazole-5-carboxamide in Example 73, Step 1. The crude material was purified by silica gel column chromatography (petroleum ether/ethyl acetate=5/1 to 2/1) to give N-(2-methyl-4-(4,4,5,5-tetramethyl-1,3,2-dioxaborolan-2-yl)benzyl)-5-(1-methylcyclopropyl)-1,2,4-oxadiazole-3-carboxamide as a yellow oil (9.6 g, 54% yield). LCMS: m/z=M+H⁺: 398.3. ¹H NMR: (400 MHz, DMSO-d₆) δ ppm 9.34 (t, J=6.0 Hz, 1H), 7.40-7.58 (m, 2H), 7.23 (d, J=7.6 Hz, 1H), 4.44 (d, J=6.0 Hz, 2H), 2.32 (s, 3H), 1.54 (s, 3H), 1.36-1.41 (m, 2H), 1.28 (s, 12H), 1.15-1.19 (m, 2H).
3. Synthesis of tert-butyl 4-(5-fluoro-4-(3-methyl-4-((5-(1-methylcyclopropyl)-1,2,4-oxadiazole-3-carboxamido)methyl)phenyl)pyridin-3-yl)piperazine-1-carboxylate

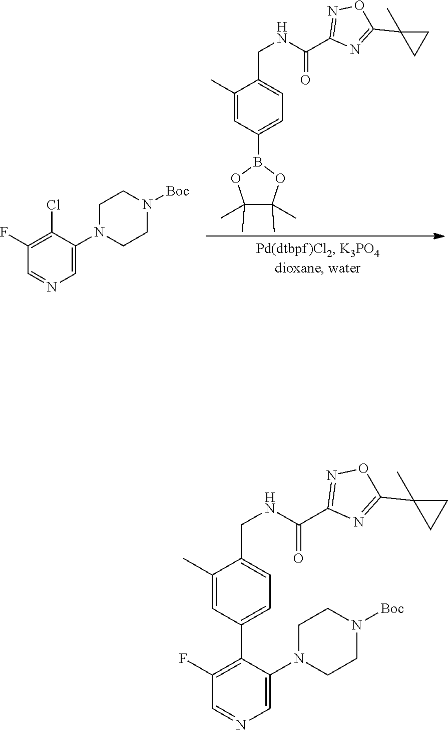 [SMILES: CC(C)(C)OC(=O)N1CCN(c2cncc(F)c2Cl)CC1 ; Cc1cc(B2OC(C)(C)C(C)(C)O2)ccc1CNC(=O)c1noc(C2(C)CC2)n1 ; Cc1cc(-c2c(F)cncc2N2CCN(C(=O)OC(C)(C)C)CC2)ccc1CNC(=O)c1noc(C2(C)CC2)n1]

4. Synthesis of tert-butyl 4-(5-fluoro-4-(3-methyl-4-((5-(-methylcyclopropyl)-1,2,4-oxadiazole-3-carboxamido)methyl)phenyl)pyridin-3-yl)piperazine-1-carboxylate was similar to that of tert-butyl 4-(4-(4-((5-(tert-butyl)-1,2,4-oxadiazole-3-carboxamido)methyl)-3-methylphenyl)-5-fluoropyridin-3-yl)piperazine-1-carboxylate in Example 76, Step 2. The crude material was purified by prep-TLC (petroleum ether/ethyl acetate) to give tert-butyl 4-(5-fluoro-4-(3-methyl-4-((5-(1-methylcyclopropyl)-1,2,4-oxadiazole-3-carboxamido)methyl)-phenyl)pyridin-3-yl)piperazine-1-carboxylate as a yellow oil (400 mg, crude), which was carried forward without further purification. LCMS: m/z=M+H⁺: 551.3.
5. Synthesis of N-(4-(3-fluoro-5-(piperazin-1-yl)pyridin-4-yl)-2-methylbenzyl)-5-(1-methylcyclopropyl)-1,2,4-oxadiazole-3-carboxamide hydrochloride

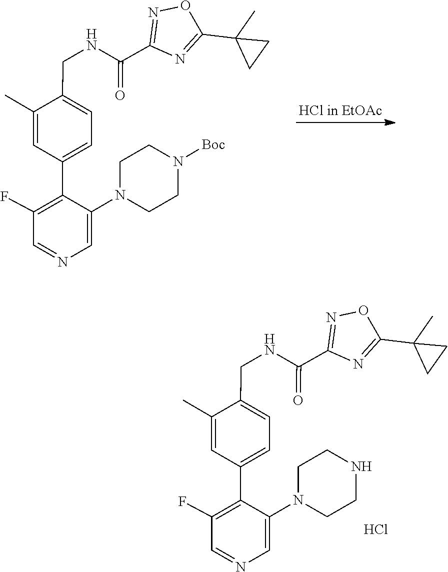 [SMILES: Cc1cc(-c2c(F)cncc2N2CCN(C(=O)OC(C)(C)C)CC2)ccc1CNC(=O)c1noc(C2(C)CC2)n1 ; Cc1cc(-c2c(F)cncc2N2CCNCC2)ccc1CNC(=O)c1noc(C2(C)CC2)n1 ; Cl]

6. Synthesis of N-(4-(3-fluoro-5-(piperazin-1-yl)pyridin-4-yl)-2-methylbenzyl)-5-(1-methylcyclopropyl)-1,2,4-oxadiazole-3-carboxamide hydrochloride was similar to that of 5-(tert-butyl)-N-(4-(3-fluoro-5-(piperazin-1-yl)pyridin-4-yl)-2-methylbenzyl)-1,2,4-oxadiazole-3-carboxamide hydrochloride in Example 76, Step 3. The reaction mixture was dried under vacuum to give N-(4-(3-fluoro-5-(piperazin-1-yl)pyridin-4-yl)-2-methylbenzyl)-5-(1-methylcyclopropyl)-1,2,4-oxadiazole-3-carboxamide hydrochloride as a yellow solid (150 mg, crude), which was carried forward without further purification. LCMS: m/z=M+H⁺: 451.3. 7. Synthesis of (E)-N-(4-(3-(4-(4-(dimethylamino)but-2-enoyl)piperazin-1-yl)-5-fluoropyridin-4-yl)-2-methylbenzyl)-5-(1-methylcyclopropyl)-1,2,4-oxadiazole-3-carboxamide

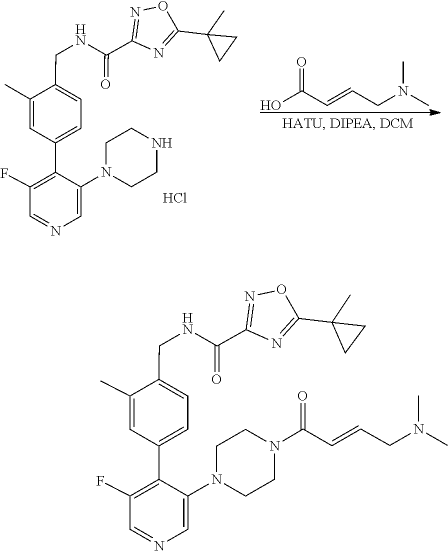

8. Synthesis of (E)-N-(4-(3-(4-(4-(dimethylamino)but-2-enoyl)piperazin-1-yl)-5-fluoropyridin-4-yl)-2-methylbenzyl)-5-(1-methylcyclopropyl)-1,2,4-oxadiazole-3-carboxamide was similar to that of 5-(tert-butyl)-N-(2-methyl-4-(3-(4-(oxirane-2-carbonyl)piperazin-1-yl)pyridin-4-yl)benzyl)-1,2,4-oxadiazole-3-carboxamide in Example 59. The crude material was purified by prep-HPLC (Column: Welch Xtimate C18 150×25 mm×5 μm; Condition: water (10 mM NH₄HCO₃)-ACN, Begin B 32, End B 62, Gradient Time (min) 10, 100% B Hold Time (min) 2, Flow Rate (mL/min) 25.) to give (E)-N-(4-(3-(4-(4-(dimethylamino)but-2-enoyl)piperazin-1-yl)-5-fluoropyridin-4-yl)-2-methylbenzyl)-5-(1-methylcyclopropyl)-1,2,4-oxadiazole-3-carboxamide as a yellow solid (54 mg, 33% yield). LCMS: m/z=M+H⁺: 562.4. ¹H NMR: (500 MHz, DMSO-d₆) δ=9.42-9.38 (m, 1H), 8.33 (s, 1H), 8.21 (s, 1H), 7.41-7.33 (m, 3H), 6.62-6.52 (m, 2H), 4.49 (d, J=6.0 Hz, 2H), 3.40 (s, 4H), 2.99 (d, J=4.5 Hz, 2H), 2.84-2.81 (m, 4H), 2.38 (s, 3H), 2.12 (d, J=1.5 Hz, 6H), 1.55 (s, 3H), 1.41-1.38 (m, 2H), 1.19-1.16 (m, 2H).

### Example 78: (E)-3-(tert-butyl)-N-(4-(3-(4-(4-(dimethylamino)but-2-enoyl)piperazin-1-yl)-5-fluoropyridin-4-yl)-2-methylbenzyl)-1,2,4-oxadiazole-5-carboxamide

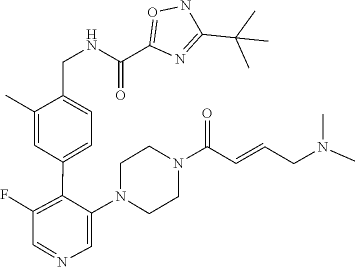 [0852] 1. Synthesis of tert-butyl 4-(4-(4-((3-(tert-butyl)-1,2,4-oxadiazole-5-carboxamido)methyl)-3-methylphenyl)-5-fluoropyridin-3-yl)piperazine-1-carboxylate

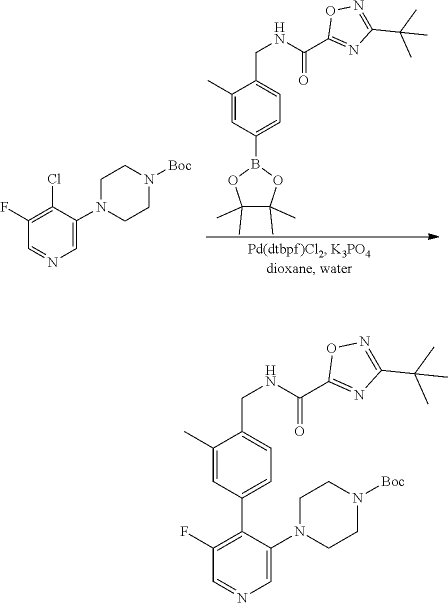 [SMILES: CC(C)(C)OC(=O)N1CCN(c2cncc(F)c2Cl)CC1 ; Cc1cc(B2OC(C)(C)C(C)(C)O2)ccc1CNC(=O)c1nc(C(C)(C)C)no1 ; Cc1cc(-c2c(F)cncc2N2CCN(C(=O)OC(C)(C)C)CC2)ccc1CNC(=O)c1nc(C(C)(C)C)no1]

2. Synthesis of tert-butyl 4-(4-(4-((3-(tert-butyl)-1,2,4-oxadiazole-5-carboxamido)methyl)-3-methylphenyl)-5-fluoropyridin-3-yl)piperazine-1-carboxylate was similar to that of tert-butyl 4-(4-(4-((5-(tert-butyl)-1,2,4-oxadiazole-3-carboxamido)methyl)-3-methylphenyl)-5-fluoropyridin-3-yl)piperazine-1-carboxylate in Example 76, Step 2. The crude material was purified by prep-TLC (petroleum ether/ethyl acetate=1/1) to give tert-butyl 4-(4-(4-((3-(tert-butyl)-1,2,4-oxadiazole-5-carboxamido)methyl)-3-methylphenyl)-5-fluoropyridin-3-yl)piperazine-1-carboxylate as a yellow oil (130 mg, 49%). LCMS: m/z=M+H⁺: 553.4.
3. Synthesis of 3-(tert-butyl)-N-(4-(3-fluoro-5-(piperazin-1-yl)pyridin-4-yl)-2-methylbenzyl)-1,2,4-oxadiazole-5-carboxamide hydrochloride

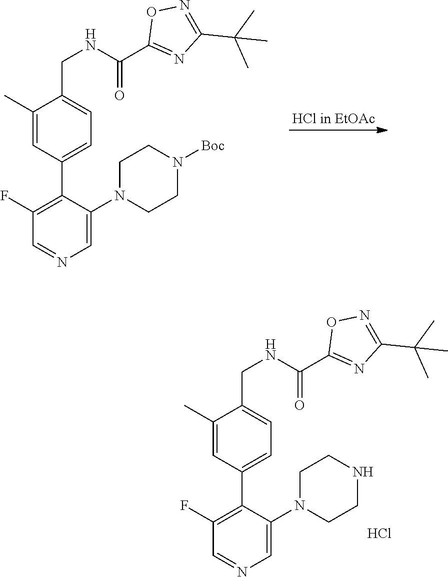 [SMILES: Cc1cc(-c2c(F)cncc2N2CCN(C(=O)OC(C)(C)C)CC2)ccc1CNC(=O)c1nc(C(C)(C)C)no1 ; Cc1cc(-c2c(F)cncc2N2CCNCC2)ccc1CNC(=O)c1nc(C(C)(C)C)no1 ; Cl]

4. Synthesis of 3-(tert-butyl)-N-(4-(3-fluoro-5-(piperazin-1-yl)pyridin-4-yl)-2-methylbenzyl)-1,2,4-oxadiazole-5-carboxamide hydrochloride was similar to that of 5-(tert-butyl)-N-(4-(3-fluoro-5-(piperazin-1-yl)pyridin-4-yl)-2-methylbenzyl)-1,2,4-oxadiazole-3-carboxamide hydrochloride in Example 76, Step 3. The reaction mixture was concentrated under vacuum to give 3-(tert-butyl)-N-(4-(3-fluoro-5-(piperazin-1-yl)pyridin-4-yl)-2-methylbenzyl)-1,2,4-oxadiazole-5-carboxamide hydrochloride as a yellow solid (100 mg, crude), which was carried forward without further purification. LCMS: m/z=M+H⁺: 453.3.
5. Synthesis of (E)-3-(tert-butyl)-N-(4-(3-(4-(4-(dimethylamino)but-2-enoyl)piperazin-1-yl)-5-fluoropyridin-4-yl)-2-methylbenzyl)-1,2,4-oxadiazole-5-carboxamide

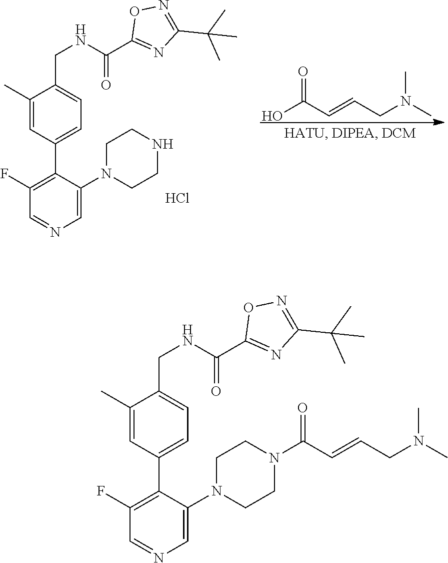

6. Synthesis of (E)-3-(tert-butyl)-N-(4-(3-(4-(4-(dimethylamino)but-2-enoyl)piperazin-1-yl)-5-fluoropyridin-4-yl)-2-methylbenzyl)-1,2,4-oxadiazole-5-carboxamide was similar to that of 5-(tert-butyl)-N-(2-methyl-4-(3-(4-(oxirane-2-carbonyl)piperazin-1-yl)pyridin-4-yl)benzyl)-1,2,4-oxadiazole-3-carboxamide in Example 59. The crude material was purified by prep-HPLC (Column: Welch Xtimate C18 150×25 mm×5 μm; Condition: water (10 mM NH₄HCO₃)-ACN, Begin B 33, End B 63, Gradient Time (min) 10, 100% B Hold Time (min) 2, Flow Rate (mL/min) 25.) to give (E)-3-(tert-butyl)-N-(4-(3-(4-(4-(dimethylamino)but-2-enoyl)piperazin-1-yl)-5-fluoropyridin-4-yl)-2-methylbenzyl)-1,2,4-oxadiazole-5-carboxamide as a yellow solid (75 mg, 72% yield). LCMS: m/z=M+H⁺: 564.4. ¹H NMR: (500 MHz, DMSO-d₆) δ=9.86-9.82 (m, 1H), 8.33 (s, 1H), 8.21 (s, 1H), 7.42-7.37 (m, 3H), 6.62-6.52 (m, 2H), 4.52 (d, J=6.0 Hz, 2H), 3.41-3.39 (m, 4H), 2.99 (d, J=5.0 Hz, 2H), 2.84-2.82 (m, 4H), 2.41-2.36 (m, 3H), 2.15-2.10 (m, 6H), 1.37 (s, 9H).

### Example 79: N-(4-(5-(4-acryloylpiperazin-1-yl)pyrimidin-4-yl)-2-methylbenzyl)-2-(tert-butyl)-2H-tetrazole-5-carboxamide

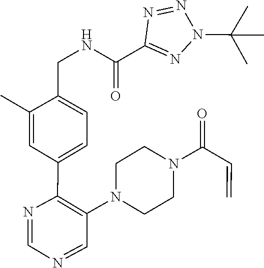 [SMILES: C=CC(=O)N1CCN(c2cncnc2-c2ccc(CNC(=O)c3nnn(C(C)(C)C)n3)c(C)c2)CC1]

### 1. Synthesis of 2-(tert-butyl)-N-(2-methyl-4-(4,4,5,5-tetramethyl-1,3,2-dioxaborolan-2-yl)benzyl)-2H-tetrazole-5-carboxamide

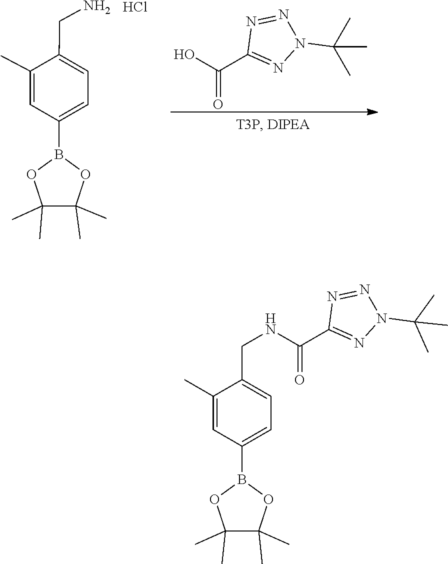 [0860] 2. Synthesis of 2-(tert-butyl)-N-(2-methyl-4-(4,4,5,5-tetramethyl-1,3,2-dioxaborolan-2-yl)benzyl)-2H-tetrazole-5-carboxamide was similar to that of N-(2-methyl-4-(4,4,5,5-tetramethyl-1,3,2-dioxaborolan-2-yl)benzyl)-3-(1-methylcyclopropyl)-1,2,4-oxadiazole-5-carboxamide in Example 73, Step 1. The crude material was purified by silica gel column chromatography (petroleum ether/ethyl acetate=10/1) to give 2-(tert-butyl)-N-(2-methyl-4-(4,4,5,5-tetramethyl-1,3,2-dioxaborolan-2-yl)benzyl)-2H-tetrazole-5-carboxamide as a colorless oil (15.5 g, 71% yield). LCMS: m/z=M+H⁺: 400.3. ¹HNMR: (400 MHz, CDCl₃) δ: 7.63-7.71 (m, 2H), 7.35 (d, J=7.20 Hz, 1H), 7.26-7.33 (br s, 1H), 4.74 (d, J=6.4 Hz, 2H), 2.41 (s, 3H), 1.81 (s, 9H), 1.37 (s, 12H).

### 3. Synthesis of tert-butyl 4-(4-(4-((2-(tert-butyl)-2H-tetrazole-5-carboxamido)methyl)-3-methylphenyl)pyrimidin-5-yl)piperazine-1-carboxylate

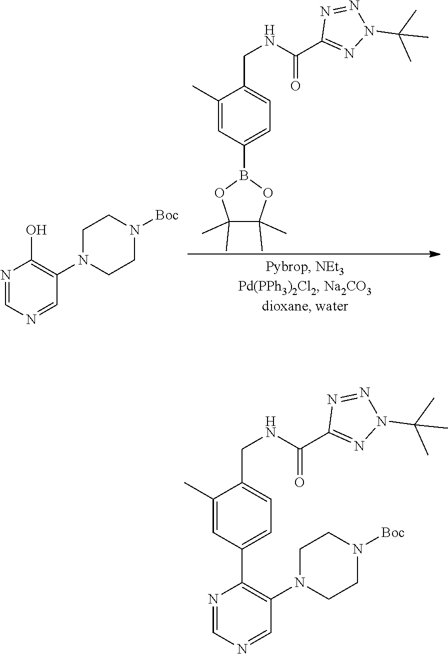

To a solution of tert-butyl 4-(4-hydroxypyrimidin-5-yl)piperazine-1-carboxylate (250 mg, 892 μmol) in dioxane (10 mL) was added triethylamine (271 mg, 2.68 mmol) and bromotripyrrolidinophosphonium hexafluorophosphate (PyBroP) (624 mg, 1.34 mmol) at 20° C. The mixture was stirred at 20° C. for 1 hour. Then 2-(tert-butyl)-N-(2-methyl-4-(4,4,5,5-tetramethyl-1,3,2-dioxaborolan-2-yl)benzyl)-2H-tetrazole-5-carboxamide (356 mg, 892 μmol), Na₂CO₃ (284 mg, 2.68 mmol), Pd(PPh₃)₂Cl₂ (63 mg, 89 μmol) and water (2 mL) was added into the mixture. The mixture was stirred at 90° C. under N₂ for 10 hours. The mixture was concentrated under vacuum to give a residue. The residue was purified by silica gel chromatography (from 100% petroleum ether to 50% ethyl acetate in petroleum ether) to give tert-butyl 4-(4-(4-((2-(tert-butyl)-2H-tetrazole-5-carboxamido)methyl)-3-methylphenyl)pyrimidin-5-yl)piperazine-1-carboxylate as a yellow oil (120 mg, 25% yield). LCMS: m/z=M+H⁺: 536.5.

### 4. Synthesis of 2-(tert-butyl)-N-(2-methyl-4-(5-(piperazin-1-yl)pyrimidin-4-yl)benzyl)-2H-tetrazole-5-carboxamide hydrochloride

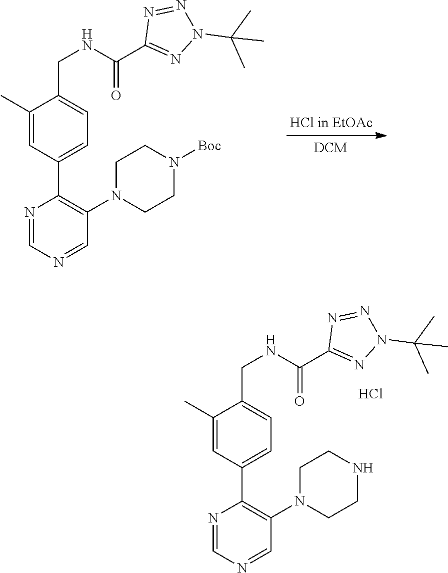

To a solution of tert-butyl 4-(4-(4-((2-(tert-butyl)-2H-tetrazole-5-carboxamido)methyl)-3-methylphenyl)pyrimidin-5-yl)piperazine-1-carboxylate (120 mg, 224 μmol) in DCM (10 mL) was added an HCl solution in ethyl acetate (8 mL, 4 M) at 20° C. The mixture was stirred at 20° C. for 1 hour. The mixture was concentrated under vacuum to give 2-(tert-butyl)-N-(2-methyl-4-(5-(piperazin-1-yl)pyrimidin-4-yl)benzyl)-2H-tetrazole-5-carboxamide hydrochloride as a yellow oil (100 mg, crude), which was carried forward without further purification. LCMS: m/z=M+H⁺: 436.4. 5. Synthesis of N-(4-(5-(4-acryloylpiperazin-1-yl)pyrimidin-4-yl)-2-methylbenzyl)-2-(tert-butyl)-2H-tetrazole-5-carboxamide

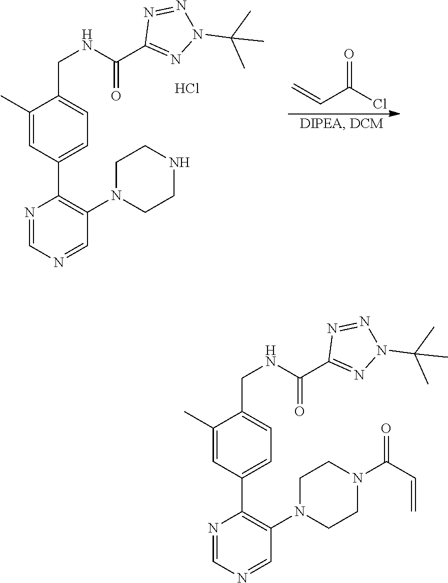

To a solution of 2-(tert-butyl)-N-(2-methyl-4-(5-(piperazin-1-yl)pyrimidin-4-yl)benzyl)-2H-tetrazole-5-carboxamide hydrochloride (100 mg, 212 μmol) in DCM (20 mL) was added DIPEA (55 mg, 424 μmol) and acryloyl chloride (19 mg, 212 μmol) at 0° C. The mixture was stirred at 0° C. for 10 minutes. The mixture was quenched with MeOH (1 mL) and concentrated under vacuum. The residue was purified by prep-HPLC (Column: Welch Xtimate C18 150×25 mm×5 μm; Condition: water (10 mM NH₄HCO₃)-ACN, Begin B 26 End B 56, Gradient Time (min) 15, 100% B Hold Time (min) 2, Flow Rate (mL/min): 25) to give N-(4-(5-(4-acryloylpiperazin-1-yl)pyrimidin-4-yl)-2-methylbenzyl)-2-(tert-butyl)-2H-tetrazole-5-carboxamide as a white solid (59 mg, 57% yield). LCMS: m/z=M+H⁺: 490.3. ¹H NMR: (500 MHz, DMSO-d₆) δ ppm=9.53 (t, J=6.0 Hz, 1H), 8.87 (s, 1H), 8.52 (s, 1H), 8.00-7.96 (m, 2H), 7.36 (d, J=8.0 Hz, 1H), 6.79 (dd, J₁=10.5 Hz, J₂=16.5 Hz, 1H), 6.12 (dd, J₁=2.0 Hz, J₂=16.5 Hz, 1H), 5.67 (dd, J₁=2.5 Hz, J₂=10.5 Hz, 1H), 4.54 (d, J=6.0 Hz, 2H), 3.60 (s, 4H), 2.91 (s, 4H), 2.41 (s, 3H), 1.74 (s, 9H).

### Example 80: N-(4-(5-(4-acryloylpiperazin-1-yl)pyrimidin-4-yl)-2-methylbenzyl)-5-(tert-butyl)-1,2,4-oxadiazole-3-carboxamide

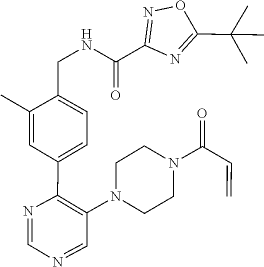 [SMILES: C=CC(=O)N1CCN(c2cncnc2-c2ccc(CNC(=O)c3noc(C(C)(C)C)n3)c(C)c2)CC1] [0867] 1. Synthesis of tert-butyl 4-(4-(4-((5-(tert-butyl)-1,2,4-oxadiazole-3-carboxamido)methyl)-3-methylphenyl) pyrimidin-5-yl)piperazine-1-carboxylate

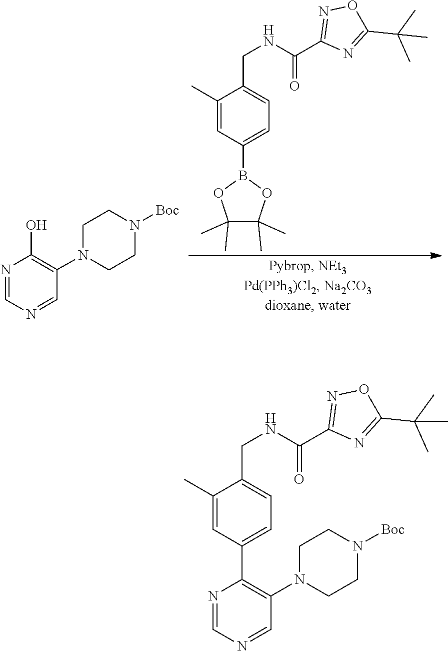 [SMILES: CC(C)(C)OC(=O)N1CCN(c2cncnc2O)CC1 ; Cc1cc(B2OC(C)(C)C(C)(C)O2)ccc1CNC(=O)c1noc(C(C)(C)C)n1 ; Cc1cc(-c2ncncc2N2CCN(C(=O)OC(C)(C)C)CC2)ccc1CNC(=O)c1noc(C(C)(C)C)n1]

2. Synthesis of tert-butyl 4-(4-(4-((5-(tert-butyl)-1,2,4-oxadiazole-3-carboxamido)methyl)-3-methylphenyl) pyrimidin-5-yl)piperazine-1-carboxylate was similar to that of tert-butyl 4-(4-(4-((2-(tert-butyl)-2H-tetrazole-5-carboxamido)methyl)-3-methylphenyl)pyrimidin-5-yl)piperazine-1-carboxylate in Example 79, Step 2. The crude material was purified by prep-TLC (petroleum ether/ethyl acetate=1/2) to give tert-butyl 4-(4-(4-((5-(tert-butyl)-1,2,4-oxadiazole-3-carboxamido)methyl)-3-methylphenyl) pyrimidin-5-yl)piperazine-1-carboxylate as a yellow oil (350 mg, 44% yield). LCMS: m/z=M+Na⁺: 558.3.
3. Synthesis of 5-(tert-butyl)-N-(2-methyl-4-(5-(piperazin-1-yl)pyrimidin-4-yl)benzyl)-1,2,4-oxadiazole-3-carboxamide hydrochloride

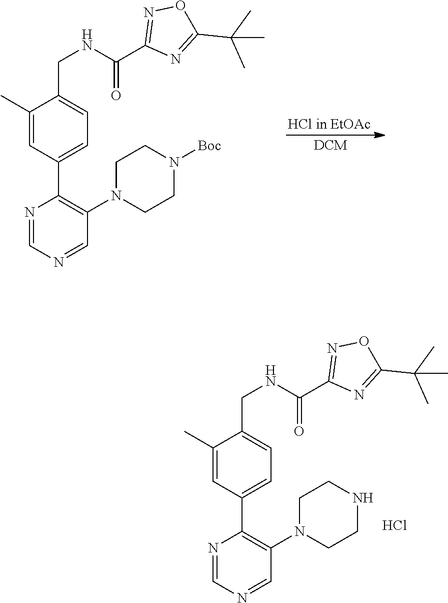 [SMILES: Cc1cc(-c2ncncc2N2CCN(C(=O)OC(C)(C)C)CC2)ccc1CNC(=O)c1noc(C(C)(C)C)n1 ; Cc1cc(-c2ncncc2N2CCNCC2)ccc1CNC(=O)c1noc(C(C)(C)C)n1 ; Cl]

4. Synthesis of 5-(tert-butyl)-N-(2-methyl-4-(5-(piperazin-1-yl)pyrimidin-4-yl)benzyl)-1,2,4-oxadiazole-3-carboxamide hydrochloride was similar to that of 2-(tert-butyl)-N-(2-methyl-4-(5-(piperazin-1-yl)pyrimidin-4-yl)benzyl)-2H-tetrazole-5-carboxamide hydrochloride in Example 79, Step 3. The reaction mixture was concentrated under vacuum to give 5-(tert-butyl)-N-(2-methyl-4-(5-(piperazin-1-yl)pyrimidin-4-yl)benzyl)-1,2,4-oxadiazole-3-carboxamide hydrochloride as a yellow solid (120 mg, crude), which was carried forward without further purification. LCMS: m/z=M+H⁺: 436.4.
5. Synthesis of N-(4-(5-(4-acryloylpiperazin-1-yl)pyrimidin-4-yl)-2-methylbenzyl)-5-(tert-butyl)-1,2,4-oxadiazole-3-carboxamide

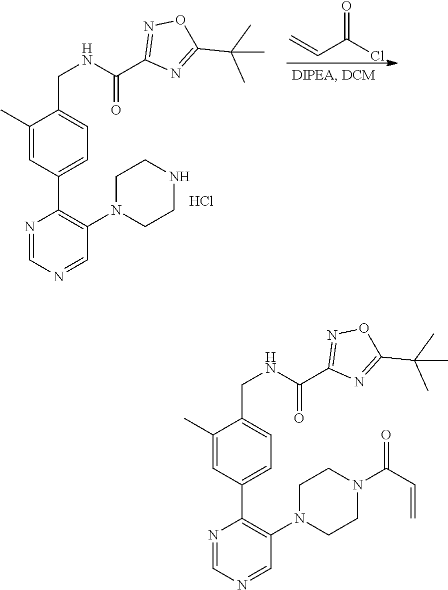

6. Synthesis of N-(4-(5-(4-acryloylpiperazin-1-yl)pyrimidin-4-yl)-2-methylbenzyl)-5-(tert-butyl)-1,2,4-oxadiazole-3-carboxamide was similar to that of N-(4-(5-(4-acryloylpiperazin-1-yl)pyrimidin-4-yl)-2-methylbenzyl)-2-(tert-butyl)-2H-tetrazole-5-carboxamide in Example 79, Step 4. The crude material was purified by prep-HPLC (Column: Welch Xtimate C18 150×25 mm×5 m; Condition: water(10 mM NH₄HCO₃)-ACN, Begin B 31, End B 61; Gradient Time (min): 10; 100% B Hold Time (min): 2; Flow Rate (ml/min): 25) to give N-(4-(5-(4-acryloylpiperazin-1-yl)pyrimidin-4-yl)-2-methylbenzyl)-5-(tert-butyl)-1,2,4-oxadiazole-3-carboxamide as a yellow solid (64 mg, 56% yield). LCMS: m/z=M+H⁺: 490.3. ¹H NMR: (400 MHz, DMSO-d₆) δ: 9.46 (t, J=6.0 Hz, 1H), 8.87 (s, 1H), 8.52 (s, 1H), 8.01-7.94 (m, 2H), 7.35 (d, J=8.0 Hz, 1H), 6.84-6.75 (m, 1H), 6.15-6.08 (m, 1H), 5.70-5.65 (m, 1H), 4.51 (d, J=6.0 Hz, 2H), 3.60 (s, 4H), 2.91 (s, 4H), 2.40 (s, 3H), 1.43 (s, 9H).

### Example 81: Synthesis of 5-(tert-butyl)-N-(4-(5-(4-(2-fluoroacryloyl)piperazin-1-yl)pyrimidin-4-yl)-2-methylbenzyl)-1,2,4-oxadiazole-3-carboxamide

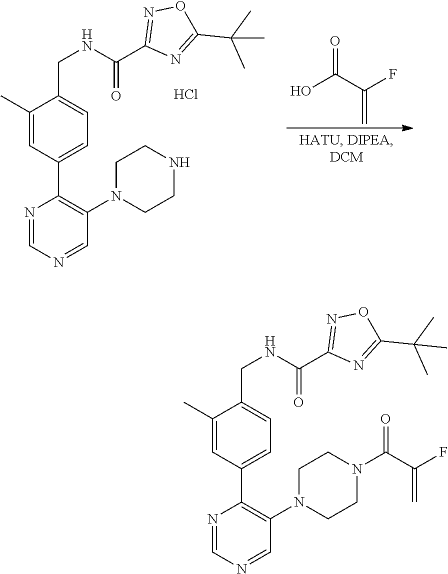

To a solution of 5-(tert-butyl)-N-(2-methyl-4-(5-(piperazin-1-yl)pyrimidin-4-yl)benzyl)-1,2,4-oxadiazole-3-carboxamide hydrochloride (130 mg, 298 μmol) in DCM (30 mL) was added DIPEA (77 mg, 597 μmol), 2-fluoroacrylic acid (27 mg, 298 μmol) and HATU (137 mg, 358 μmol) at 20° C. The mixture was stirred at 20° C. for 30 minutes. The mixture was concentrated under vacuum. The residue was purified by prep-HPLC (Column: Welch Xtimate C18 150×25 mm×5 μm; Condition: water (10 mM NH₄HCO₃)-ACN, Begin B 32 End B 62, Gradient Time (min) 10, 100% B Hold Time (min) 2, Flow Rate (mL/min): 25) to give 5-(tert-butyl)-N-(4-(5-(4-(2-fluoroacryloyl)piperazin-1-yl)pyrimidin-4-yl)-2-methylbenzyl)-1,2,4-oxadiazole-3-carboxamide as a white solid (53 mg, 35% yield). LCMS: m/z=M+H⁺: 508.3. ¹H NMR: (400 MHz, DMSO-d₆) δ ppm=9.45 (t, J=6.0 Hz, 1H), 8.88 (s, 1H), 8.53 (s, 1H), 8.01-7.95 (m, 2H), 7.35 (d, J=8.0 Hz, 1H), 5.31-5.11 (m, 2H), 4.51 (d, J=6.0 Hz, 2H), 3.57 (s, 4H), 2.95 (s, 4H), 2.40 (s, 3H), 1.43 (s, 9H).

### Example 82: Synthesis of (E)-5-(tert-butyl)-N-(4-(5-(4-(4-(dimethylamino)but-2-enoyl)piperazin-1-yl)pyrimidin-4-yl)-2-methylbenzyl)-1,2,4-oxadiazole-3-carboxamide

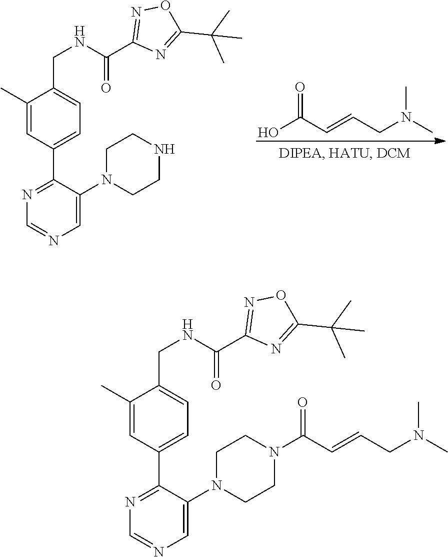 [0876] 1. Synthesis of (E)-5-(tert-butyl)-N-(4-(5-(4-(4-(dimethylamino)but-2-enoyl)piperazin-1-yl)pyrimidin-4-yl)-2-methylbenzyl)-1,2,4-oxadiazole-3-carboxamide was similar to that of 5-(tert-butyl)-N-(2-methyl-4-(3-(4-(oxirane-2-carbonyl)piperazin-1-yl)pyridin-4-yl)benzyl)-1,2,4-oxadiazole-3-carboxamide in Example 59. The crude material was purified by prep HPLC (Column: Welch Xtimate C18 150×25 mm×5 μm; Condition: water (10 mM NH4HCO3)-ACN, Begin B 27, End B 57, Gradient Time (min) 10, 100% B Hold Time (min) 2, Flow Rate (mL/min) 25.) to give (E)-5-(tert-butyl)-N-(4-(5-(4-(4-(dimethylamino)but-2-enoyl)piperazin-1-yl)pyrimidin-4-yl)-2-methylbenzyl)-1,2,4-oxadiazole-3-carboxamide as a white solid (78 mg, 48% yield). LCMS: m/z=M+H: 547.5. HNMR: (500 MHz, METHANOL-d₄) δ=8.84 (s, 1H), 8.50 (s, 1H), 7.95-7.89 (m, 2H), 7.47 (d, J=8.0 Hz, 1H), 6.81-6.73 (m, 1H), 6.62 (d, J=15.0 Hz, 1H), 4.68 (s, 2H), 3.69-3.67 (m, 4H), 3.17 (d, J=6.5 Hz, 2H), 3.03-2.97 (m, 4H), 2.49 (s, 3H), 2.29 (s, 6H), 1.51 (s, 9H).

### Example 83: N-(4-(5-(4-acryloylpiperazin-1-yl)pyrimidin-4-yl)-2-methylbenzyl)-3-(tert-butyl)-1,2,4-oxadiazole-5-carboxamide

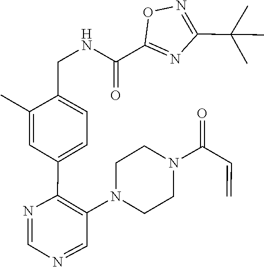 [SMILES: C=CC(=O)N1CCN(c2cncnc2-c2ccc(CNC(=O)c3nc(C(C)(C)C)no3)c(C)c2)CC1] [0878] 1. Synthesis of tert-butyl 4-(4-(4-((3-(tert-butyl)-1,2,4-oxadiazole-5-carboxamido)methyl)-3-methylphenyl) pyrimidin-5-yl)piperazine-1-carboxylate

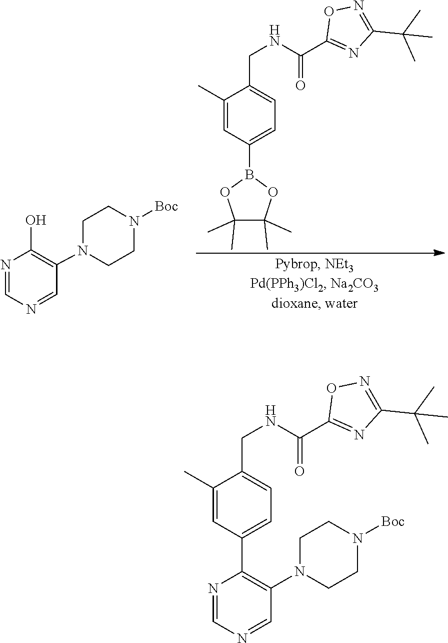 [SMILES: CC(C)(C)OC(=O)N1CCN(c2cncnc2O)CC1 ; Cc1cc(B2OC(C)(C)C(C)(C)O2)ccc1CNC(=O)c1nc(C(C)(C)C)no1 ; Cc1cc(-c2ncncc2N2CCN(C(=O)OC(C)(C)C)CC2)ccc1CNC(=O)c1nc(C(C)(C)C)no1]

2. Synthesis of tert-butyl 4-(4-(4-((3-(tert-butyl)-1,2,4-oxadiazole-5-carboxamido)methyl)-3-methylphenyl) pyrimidin-5-yl)piperazine-1-carboxylate was similar to that of tert-butyl 4-(4-(4-((2-(tert-butyl)-2H-tetrazole-5-carboxamido)methyl)-3-methylphenyl)pyrimidin-5-yl)piperazine-1-carboxylate in Example 79, Step 2. The crude material was purified by silica gel chromatography (from petroleum ether to ethyl acetate) to give tert-butyl 4-(4-(4-((3-(tert-butyl)-1,2,4-oxadiazole-5-carboxamido)methyl)-3-methylphenyl) pyrimidin-5-yl)piperazine-1-carboxylate as a yellow solid (110 mg, 26% yield). LCMS: m/z=M+H⁺: 536.5.
3. Synthesis of 3-(tert-butyl)-N-(2-methyl-4-(5-(piperazin-1-yl)pyrimidin-4-yl)benzyl)-1,2,4-oxadiazole-5-carboxamide hydrochloride

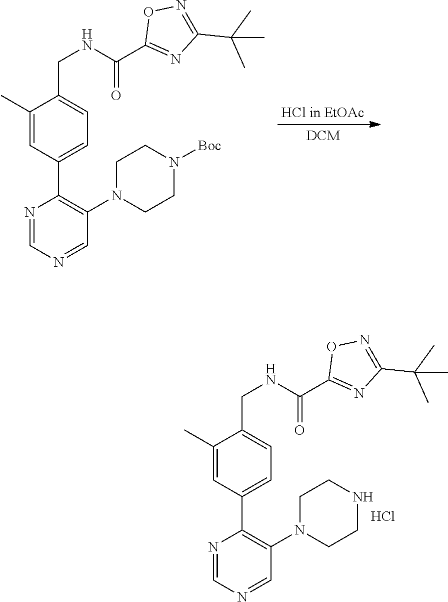 [SMILES: Cc1cc(-c2ncncc2N2CCN(C(=O)OC(C)(C)C)CC2)ccc1CNC(=O)c1nc(C(C)(C)C)no1 ; Cc1cc(-c2ncncc2N2CCNCC2)ccc1CNC(=O)c1nc(C(C)(C)C)no1 ; Cl]

4. Synthesis of 3-(tert-butyl)-N-(2-methyl-4-(5-(piperazin-1-yl)pyrimidin-4-yl)benzyl)-1,2,4-oxadiazole-5-carboxamide hydrochloride was similar to that of 2-(tert-butyl)-N-(2-methyl-4-(5-(piperazin-1-yl)pyrimidin-4-yl)benzyl)-2H-tetrazole-5-carboxamide hydrochloride in Example 79, Step 3. The reaction mixture was concentrated under vacuum to give 3-(tert-butyl)-N-(2-methyl-4-(5-(piperazin-1-yl)pyrimidin-4-yl)benzyl)-1,2,4-oxadiazole-5-carboxamide hydrochloride as a yellow solid (90 mg, crude), which was carried forward without further purification. LCMS: m/z=M+H⁺: 436.3.
5. Synthesis of N-(4-(5-(4-acryloylpiperazin-1-yl)pyrimidin-4-yl)-2-methylbenzyl)-3-(tert-butyl)-1,2,4-oxadiazole-5-carboxamide

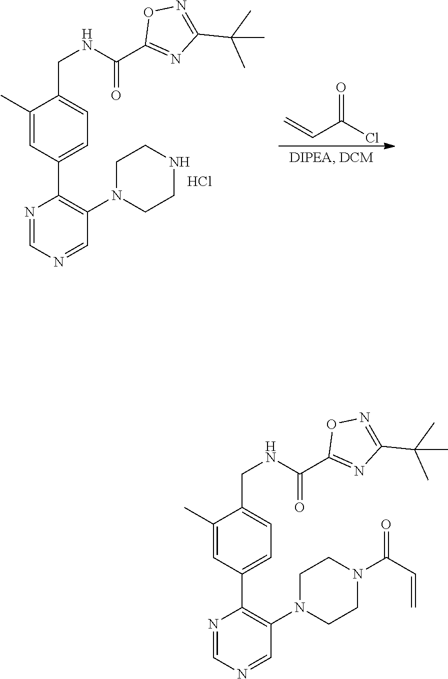

6. Synthesis of N-(4-(5-(4-acryloylpiperazin-1-yl)pyrimidin-4-yl)-2-methylbenzyl)-3-(tert-butyl)-1,2,4-oxadiazole-5-carboxamide was similar to that of N-(4-(5-(4-acryloylpiperazin-1-yl)pyrimidin-4-yl)-2-methylbenzyl)-2-(tert-butyl)-2H-tetrazole-5-carboxamide in Example 79, Step 4. The crude material was purified by prep-HPLC (Column: Welch Xtimate C18 150×25 mm×5 μm; Condition: water (10 mM NH₄HCO₃)-ACN, Begin B 32, End B 62, Gradient Time (min) 10, 100% B Hold Time (min) 2, Flow Rate (mL/min) 25.) to give N-(4-(5-(4-acryloylpiperazin-1-yl)pyrimidin-4-yl)-2-methylbenzyl)-3-(tert-butyl)-1,2,4-oxadiazole-5-carboxamide as a white solid (36 mg, 39% yield). LCMS: m/z=M+H⁺: 490.3. ¹H NMR: (400 MHz, DMSO-d₆) δ ppm=9.83-9.79 (m, 1H), 8.84 (s, 1H), 8.49 (s, 1H), 7.97-7.92 (m, 2H), 7.35 (d, J=8.0 Hz, 1H), 6.70-6.72 (m, 1H), 6.11-6.05 (m, 1H), 5.66-5.62 (m, 1H), 4.48 (d, J=6.0 Hz, 2H), 3.57 (s, 4H), 2.88 (s, 4H), 2.37 (s, 3H), 1.33 (s, 9H).

### Example 84: N-(4-(5-(4-acryloylpiperazin-1-yl)pyrimidin-4-yl)-2-methylbenzyl)-3-(1-methylcyclopropyl)-1,2,4-oxadiazole-5-carboxamide

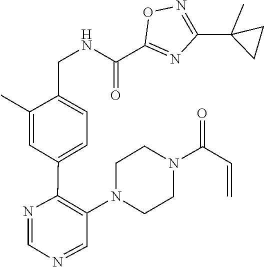 [SMILES: C=CC(=O)N1CCN(c2cncnc2-c2ccc(CNC(=O)c3nc(C4(C)CC4)no3)c(C)c2)CC1] [0885] 1. Synthesis of tert-butyl 4-(4-(3-methyl-4-((3-(1-methylcyclopropyl)-1,2,4-oxadiazole-5-carboxamido) methyl)phenyl)pyrimidin-5-yl)piperazine-1-carboxylate

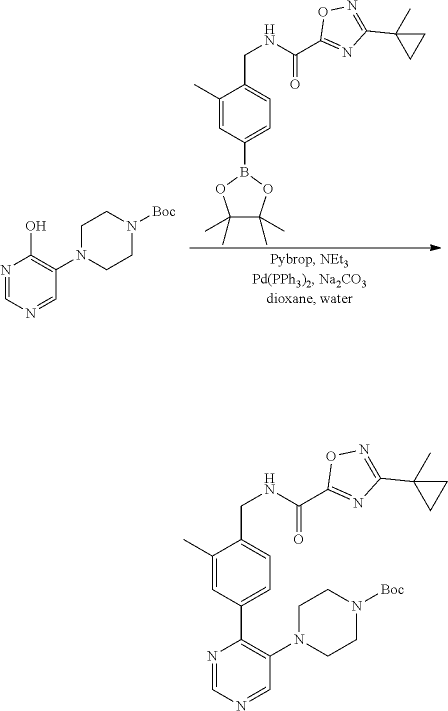 [SMILES: CC(C)(C)OC(=O)N1CCN(c2cncnc2O)CC1 ; Cc1cc(B2OC(C)(C)C(C)(C)O2)ccc1CNC(=O)c1nc(C2(C)CC2)no1 ; Cc1cc(-c2ncncc2N2CCN(C(=O)OC(C)(C)C)CC2)ccc1CNC(=O)c1nc(C2(C)CC2)no1]

2. Synthesis of tert-butyl 4-(4-(3-methyl-4-((3-(1-methylcyclopropyl)-1,2,4-oxadiazole-5-carboxamido) methyl)phenyl)pyrimidin-5-yl)piperazine-1-carboxylate was similar to that of tert-butyl 4-(4-(4-((2-(tert-butyl)-2H-tetrazole-5-carboxamido)methyl)-3-methylphenyl)pyrimidin-5-yl)piperazine-1-carboxylate in Example 779, Step 2. The crude material was purified by silica gel chromatography (from petroleum ether to ethyl acetate) to give tert-butyl 4-(4-(3-methyl-4-((3-(1-methylcyclopropyl)-1,2,4-oxadiazole-5-carboxamido) methyl)phenyl)pyrimidin-5-yl)piperazine-1-carboxylate as a yellow oil (300 mg, 54% yield). LCMS: m/z=M+H⁺: 534.3.
3. Synthesis of N-(2-methyl-4-(5-(piperazin-1-yl)pyrimidin-4-yl)benzyl)-3-(1-methylcyclopropyl)-1,2,4-oxadiazole-5-carboxamide hydrochloride

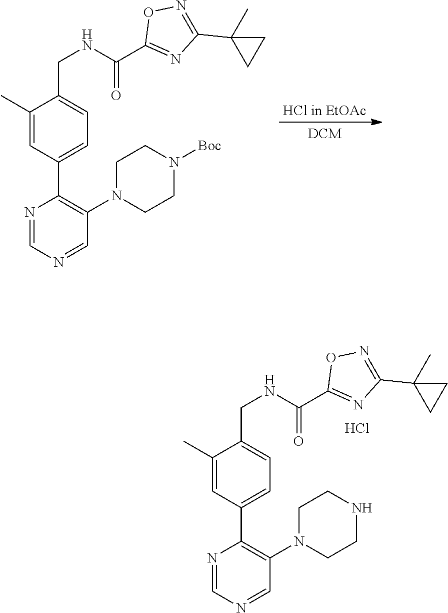 [SMILES: Cc1cc(-c2ncncc2N2CCN(C(=O)OC(C)(C)C)CC2)ccc1CNC(=O)c1nc(C2(C)CC2)no1 ; Cc1cc(-c2ncncc2N2CCNCC2)ccc1CNC(=O)c1nc(C2(C)CC2)no1 ; Cl]

4. Synthesis of N-(2-methyl-4-(5-(piperazin-1-yl)pyrimidin-4-yl)benzyl)-3-(1-methylcyclopropyl)-1,2,4-oxadiazole-5-carboxamide hydrochloride was similar to that of 2-(tert-butyl)-N-(2-methyl-4-(5-(piperazin-1-yl)pyrimidin-4-yl)benzyl)-2H-tetrazole-5-carboxamide hydrochloride in Example 79, Step 3. The reaction mixture was concentrated under vacuum to give N-(2-methyl-4-(5-(piperazin-1-yl)pyrimidin-4-yl)benzyl)-3-(1-methylcyclopropyl)-1,2,4-oxadiazole-5-carboxamide hydrochloride as a yellow oil (200 mg, crude), which was carried forward without further purification. LCMS: m/z=M+H⁺: 434.2.
5. Synthesis of N-(4-(5-(4-acryloylpiperazin-1-yl)pyrimidin-4-yl)-2-methylbenzyl)-3-(1-methylcyclopropyl)-1,2,4-oxadiazole-5-carboxamide

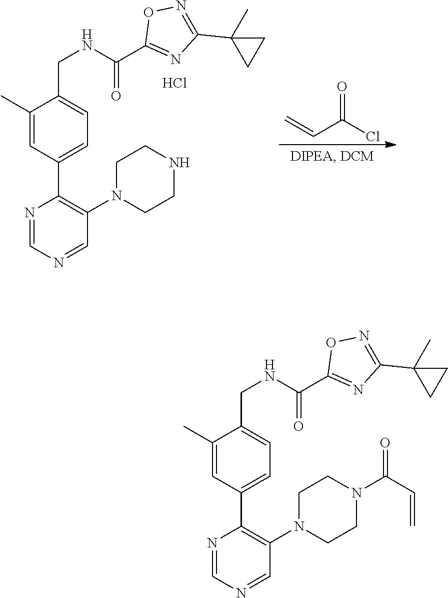

6. Synthesis of N-(4-(5-(4-acryloylpiperazin-1-yl)pyrimidin-4-yl)-2-methylbenzyl)-3-(1-methylcyclopropyl)-1,2,4-oxadiazole-5-carboxamide was similar to that of N-(4-(5-(4-acryloylpiperazin-1-yl)pyrimidin-4-yl)-2-methylbenzyl)-2-(tert-butyl)-2H-tetrazole-5-carboxamide in Example 79, Step 4. The crude material was purified by prep HPLC (Column: Welch Xtimate C18 150×25 mm×5 μm; Condition: water (10 mM NH₄HCO₃)-ACN, Begin B 29, End B 59, Gradient Time (min) 10, 100% B Hold Time (min) 2, Flow Rate (mL/min) 25) to give N-(4-(5-(4-acryloylpiperazin-1-yl)pyrimidin-4-yl)-2-methylbenzyl)-3-(1-methylcyclopropyl)-1,2,4-oxadiazole-5-carboxamide as a white solid (88 mg, 39% yield). LCMS: m/z=M+H⁺: 488.4. ¹H NMR: (500 MHz, METHANOL-d₄) δ=8.84 (s, 1H), 8.50 (s, 1H), 7.94-7.90 (m, 2H), 7.46 (d, J=8.0 Hz, 1H), 6.75 (dd, J₁=10.5 Hz, J₂=16.5 Hz, 1H), 6.22 (dd, J₁=2.0 Hz, J₂=17.0 Hz, 1H), 5.76 (dd, J₁=2.0 Hz, J₂=11.0 Hz, 1H), 4.65 (s, 2H), 3.68 (s, 4H), 3.01-2.98 (m, 4H), 2.48 (s, 3H), 1.55 (s, 3H), 1.32-1.29 (m, 2H), 0.99-0.96 (m, 2H).

### Example 85: Synthesis of (E)-N-(4-(5-(4-(4-(dimethylamino)but-2-enoyl)piperazin-1-yl)pyrimidin-4-yl)-2-methylbenzyl)-3-(1-methylcyclopropyl)-1,2,4-oxadiazole-5-carboxamide

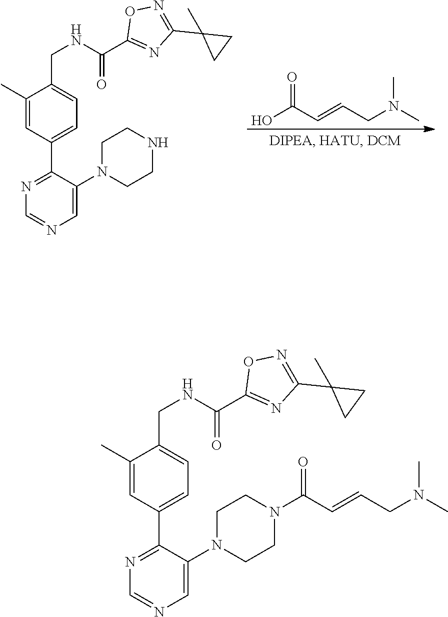 [0892] 1. Synthesis of E)-N-(4-(5-(4-(4-(dimethylamino)but-2-enoyl)piperazin-1-yl)pyrimidin-4-yl)-2-methylbenzyl)-3-(1-methylcyclopropyl)-1,2,4-oxadiazole-5-carboxamide was similar to that of 5-(tert-butyl)-N-(2-methyl-4-(3-(4-(oxirane-2-carbonyl)piperazin-1-yl)pyridin-4-yl)benzyl)-1,2,4-oxadiazole-3-carboxamide in Example 59. The crude material was purified by prep HPLC (Column: Welch Xtimate C18 150×25 mm×5 μm; Condition: water (10 mM NH₄HCO₃)-ACN, Begin B 29, End B 59, Gradient Time (min) 10, 100% B Hold Time (min) 2, Flow Rate (mL/min) 25) to give (E)-N-(4-(5-(4-(4-(dimethylamino)but-2-enoyl)piperazin-1-yl)pyrimidin-4-yl)-2-methylbenzyl)-3-(1-methylcyclopropyl)-1,2,4-oxadiazole-5-carboxamide as a white solid (121 mg, 73% yield). LCMS: m/z=M+H⁺: 545.3. ¹H NMR: (500 MHz, METHANOL-d₄) δ=8.83 (s, 1H), 8.50 (s, 1H), 7.96-7.88 (m, 2H), 7.46 (d, J=8.0 Hz, 1H), 6.81-6.73 (m, 1H), 6.61 (d, J=15.0 Hz, 1H), 4.65 (s, 2H), 3.69-3.66 (m, 4H), 3.18-3.15 (m, 2H), 3.03-2.96 (m, 4H), 2.48 (s, 3H), 2.32-2.23 (m, 6H), 1.55 (s, 3H), 1.33-1.27 (m, 2H), 1.01-0.93 (m, 2H).

### Example 86: (R,E)-5-(tert-butyl)-N-(1-(4-(3-(4-(4-(dimethylamino)but-2-enoyl)piperazin-1-yl)pyridin-4-yl)-2-methylphenyl)ethyl)-1,2,4-oxadiazole-3-carboxamide

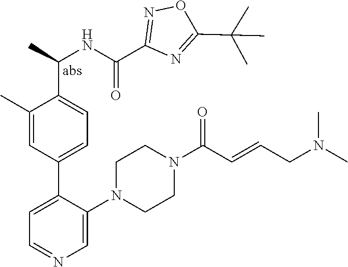

### 1. Synthesis of 1-(4-bromo-2-methylphenyl)ethan-1-one

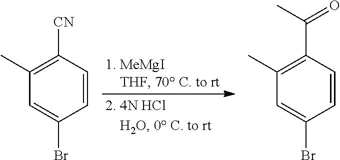

To a solution of 4-bromo-2-methyl-benzonitrile (4.0 g, 20.4 mmol) in THF (20 mL) was added dropwise iodo(methyl)magnesium (3 M, 10.2 mL) at rt. The reaction mixture was heated to reflux (−70° C.) for 2 h and then was cooled to rt and stirred for 72 h. The reaction mixture was placed in an ice-water cooling bath and saturated aqueous NH₄Cl solution (100 mL) was added, followed by EtOAc (100 mL). The layers were separated, and the aqueous phase was extracted with EtOAc (100 mL). The combined organic extracts were concentrated in vacuo and the resulting residue was treated with an HCl solution (4 N, 20 mL) at 0° C. The mixture was then stirred at rt for 18 h. The reaction mixture was extracted with EtOAc (50 mL) and the organic layer was washed with H₂O (50 mL), dried (Na₂SO₄), and filtered. The filtrate was concentrated in vacuo to give 1-(4-bromo-2-methylphenyl)ethan-1-one as a pale orange oil (3.04 g, yield: 70%). ESI-MS (M+H)⁺: 213.0. ¹H NMR (500 MHz, CDCl₃) δ: 7.57 (d, J=7.9 Hz, 1H), 7.43-7.40 (m, 2H), 2.56 (s, 3H), 2.52 (s, 3H).

### 2. Synthesis of (R,E)-N-(1-(4-bromo-2-methylphenyl)ethylidene)-2-methylpropane-2-sulfinamide

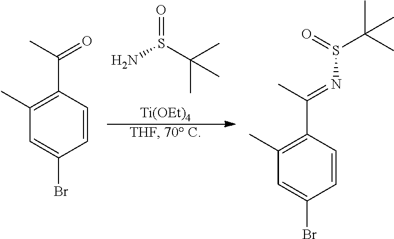

To a solution of 1-(4-bromo-2-methylphenyl)ethan-1-one (3.04 g, 14.3 mmol) in THF (48 mL) were added (R)-(+)-2-methyl-2-propanesulfinamide (1.73 g, 14.3 mmol) and Ti(IV)(OEt)₄ (6.51 g, 28.5 mmol, 5.97 mL). The reaction mixture was heated to 70° C. for 20 h. After cooling to rt, the mixture was quenched with brine (100 mL) and EtOAc (100 mL) was added, affording a biphasic solution with a thick white precipitate. The solution was filtered, and the solids were washed with EtOAc (100 mL). The filtrate layers were separated, and the organic layer was dried (Na₂SO₄), filtered and concentrated in vacuo. The crude material was purified by silica-gel column chromatography (ethyl acetate/heptanes, grading from 0% to 50%) to give (R,E)-N-(1-(4-bromo-2-methylphenyl)ethylidene)-2-methylpropane-2-sulfinamide as a yellow oil (2.96 g, yield: 66%). ESI-MS (M+H)⁺: 318.0.

### 3. Synthesis of (R)-N—((R)-1-(4-bromo-2-methylphenyl)ethyl)-2-methylpropane-2-sulfinamide

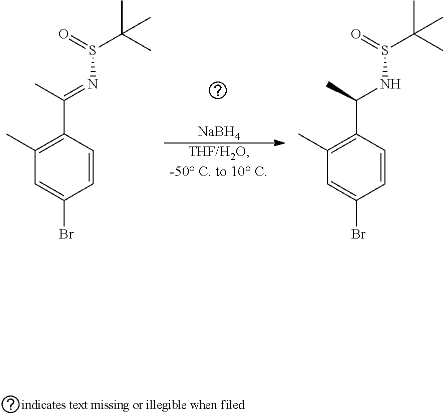 [SMILES: C/C(=N\[S@](=O)C(C)(C)C)c1ccc(Br)cc1C ; "NaBH4" ; "THF/H2O,-50° C. to 10° C." ; Cc1cc(Br)ccc1[C@@H](C)N[S@](=O)C(C)(C)C]

To a solution of (R,E)-N-(1-(4-bromo-2-methylphenyl)ethylidene)-2-methylpropane-2-sulfinamide (2.96 g, 9.4 mmol) in a mixture of THF/H₂O (98/2, 62.4 mL) cooled to −50° C. in a dry ice/acetonitrile cooling bath was added NaBH₄ (1.06 g, 28.1 mmol) slowly portion-wise. The mixture was stirred at −50° C. for 7 hours, then was stirred for 18 h as the internal temperature warmed to 10° C. The reaction was quenched with H₂O (20 mL) and diluted with EtOAc (100 mL). The layers were separated, and the organic phase was dried (Na₂SO₄), filtered, and concentrated in vacuo. The crude material was purified by silica-gel column chromatography (EtOAc/heptanes, grading from 20% to 100%) to give (R)-N—((R)-1-(4-bromo-2-methylphenyl)ethyl)-2-methylpropane-2-sulfinamide as a colorless oil (1.6 g, yield: 55%). ESI-MS (M+H)⁺: 320.1. ¹H NMR (400 MHz, CDCl₃) δ: ppm 7.37-7.33 (m, 1H), 7.33-7.31 (m, 1H), 7.29-7.25 (m, 1H), 4.78-4.68 (m, 1H), 3.31 (br s, 1H), 2.36 (s, 3H), 1.47 (d, J=6.5 Hz, 3H), 1.24 (s, 9H).

### 4. Synthesis of (R)-1-(4-bromo-2-methylphenyl)ethan-1-amine hydrochloride

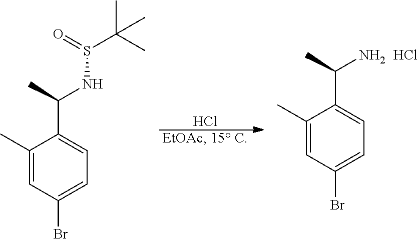

To a solution of (R)-N—((R)-1-(4-bromo-2-methylphenyl)ethyl)-2-methylpropane-2-sulfinamide (4.5 g, 14.1 mmol) in EtOAc (5 mL) at 15° C. was added an HCl/EtOAc solution (4 M, 30 mL). The reaction mixture was stirred for 2 h and filtered. The filter cake was dried under vacuum to give (R)-1-(4-bromo-2-methylphenyl)ethan-1-amine hydrochloride as a white solid (3.3 g, yield: 93%), which was carried forward without further purification. ESI-MS (M-NH₂)+: 198.9. ¹H NMR (400 MHz, DMSO-d₆) δ: 8.54 (s, 1H), 7.53-7.50 (m, 2H), 7.47 (s, 1H), 4.47 (t, J=6.0 Hz, 1H), 2.33 (s, 3H), 1.43 (d, J=6.4 Hz, 3H).

### 5. Synthesis of tert-butyl (R)-(1-(4-bromo-2-methylphenyl)ethyl)carbamate

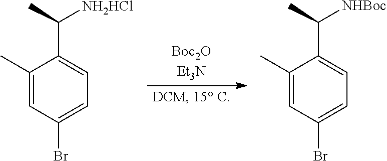

To a mixture of (R)-1-(4-bromo-2-methylphenyl)ethan-1-amine hydrochloride (3.3 g, 13.2 mmol) in DCM (40 mL) at 15° C. was added Et₃N (2.67 g, 26.3 mmol) and Boc₂O (3.7 g, 17.1 mmol). The mixture was stirred at 15° C. for 17 h, concentrated in vacuo, and purified by silica-gel column chromatography (petroleum ether/EtOAc, 20:1) to give tert-butyl (R)-(1-(4-bromo-2-methylphenyl)ethyl)carbamate as a white solid (3.8 g, yield: 92%). ESI-MS (M-Boc-NH₂)+: 198.8.

### 6. Synthesis of tert-butyl (R)-(1-(2-methyl-4-(4,4,5,5-tetramethyl-1,3,2-dioxaborolan-2-yl)phenyl)ethyl)carbamate

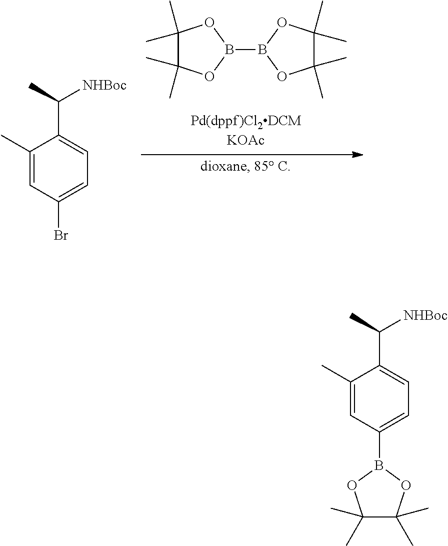

To a solution of tert-butyl (R)-(1-(4-bromo-2-methylphenyl)ethyl)carbamate (3.8 g, 12.1 mmol) in 1,4-dioxane (30 mL) under N₂ were added bis(pinacolato)diboron (3.69 g, 14.5 mmol), Pd(dppf)Cl₂•DCM (987 mg, 1.2 mmol) and KOAc (2.37 g, 24.2 mmol). The mixture was heated to 85° C. under N₂ and stirred at that temperature for 17 h, cooled to rt, and concentrated in vacuo. The crude material was purified by silica-gel column chromatography (petroleum ether/EtOAc, 20:1) to give tert-butyl (R)-(1-(2-methyl-4-(4,4,5,5-tetramethyl-1,3,2-dioxaborolan-2-yl)phenyl)ethyl)carbamate as a yellow oil (4.0 g, yield: 87%). ¹H NMR: (400 MHz, CD₃OD) δ: 7.53 (d, J=7.6 Hz, 1H), 7.50 (s, 1H), 7.30 (d, J=7.6 Hz, 1H), 3.65 (s, 1H), 2.37 (s, 3H), 1.41 (s, 9H), 1.33 (s, 12H), 1.25-1.22 (m, 3H).

### 7. Synthesis of (R)-1-(2-methyl-4-(4,4,5,5-tetramethyl-1,3,2-dioxaborolan-2-yl)phenyl)ethan-1-amine hydrochloride

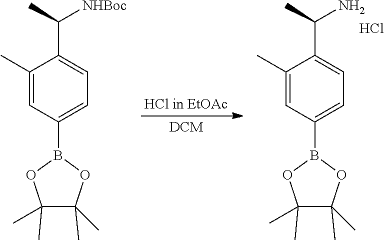

To a solution of tert-butyl (R)-(1-(2-methyl-4-(4,4,5,5-tetramethyl-1,3,2-dioxaborolan-2-yl)phenyl)ethyl)carbamate (480 mg, 1.33 mmol) in DCM (15 mL) was added an HCl solution in ethyl acetate (10 mL, 4 M) at 20° C. The mixture was stirred at 20° C. for 1 hour. The mixture was concentrated under vacuum to give (R)-1-(2-methyl-4-(4,4,5,5-tetramethyl-1,3,2-dioxaborolan-2-yl)phenyl)ethan-1-amine hydrochloride as a white solid (400 mg, crude), which was carried forward without further purification. ESI-MS (M+H)⁺: 262.3.

### 8. Synthesis of (R)-5-(tert-butyl)-N-(1-(2-methyl-4-(4,4,5,5-tetramethyl-1,3,2-dioxaborolan-2-yl)phenyl)ethyl)-1,2,4-oxadiazole-3-carboxamide

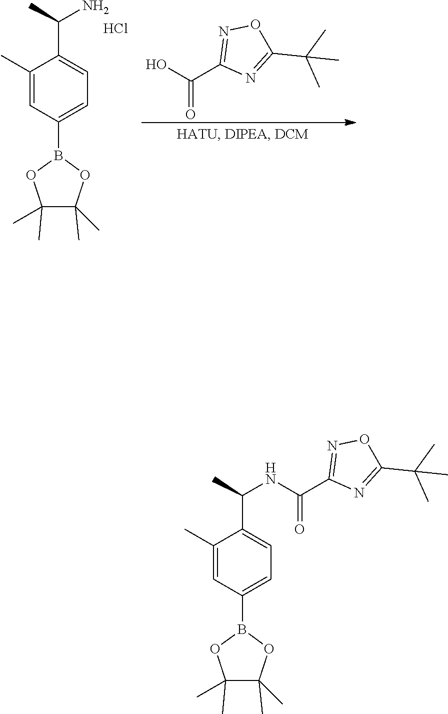

To a solution of (R)-1-(2-methyl-4-(4,4,5,5-tetramethyl-1,3,2-dioxaborolan-2-yl)phenyl)ethan-1-amine hydrochloride (400 mg, 1.34 mmol) in DCM (100 mL) was added DIPEA (347 mg, 2.69 mmol), 5-(tert-butyl)-1,2,4-oxadiazole-3-carboxylic acid (343 mg, 2.02 mmol) and HATU (512 mg, 1.34 mmol) at 20° C. The mixture was stirred at 20° C. for 1 hour. The mixture was concentrated under vacuum to give a residue. The residue was purified by silica gel chromatography (grading from petroleum ether to petroleum ether/ethyl acetate=3/1) to give (R)-5-(tert-butyl)-N-(1-(2-methyl-4-(4,4,5,5-tetramethyl-1,3,2-dioxaborolan-2-yl)phenyl)ethyl)-1,2,4-oxadiazole-3-carboxamide as a yellow oil (200 mg, 36% yield) as yellow oil. ESI-MS (M+H)⁺: 414.3.

### 9. Synthesis of tert-butyl (R)-4-(4-(4-(1-(5-(tert-butyl)-1,2,4-oxadiazole-3-carboxamido)ethyl)-3-methylphenyl)pyridin-3-yl)piperazine-1-carboxylate

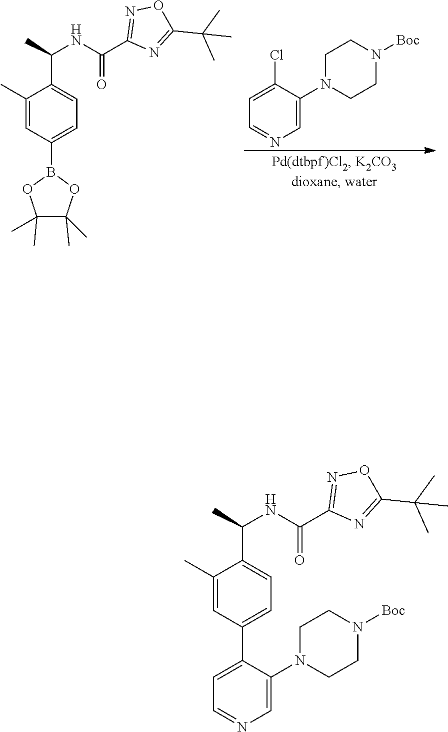 [SMILES: Cc1cc(B2OC(C)(C)C(C)(C)O2)ccc1[C@@H](C)NC(=O)c1noc(C(C)(C)C)n1 ; CC(C)(C)OC(=O)N1CCN(c2cnccc2Cl)CC1 ; Cc1cc(-c2ccncc2N2CCN(C(=O)OC(C)(C)C)CC2)ccc1[C@@H](C)NC(=O)c1noc(C(C)(C)C)n1]

To a solution of (R)-5-(tert-butyl)-N-(1-(2-methyl-4-(4,4,5,5-tetramethyl-1,3,2-dioxaborolan-2-yl)phenyl)ethyl)-1,2,4-oxadiazole-3-carboxamide (170 mg, 411 μmol) in dioxane (10 mL) and water (2 mL) was added tert-butyl 4-(4-chloropyridin-3-yl)piperazine-1-carboxylate (122 mg, 411 μmol), K₂CO₃ (114 mg, 823 μmol) and Pd(dtbpf)Cl₂ (27 mg, 41 μmol) at 20° C. The mixture was stirred at 90° C. under N₂ for 2 hours. The mixture was concentrated under vacuum to give a residue. The residue was purified by silica gel chromatography (from petroleum ether to ethyl acetate) to give tert-butyl (R)-4-(4-(4-(1-(5-(tert-butyl)-1,2,4-oxadiazole-3-carboxamido)ethyl)-3-methylphenyl)pyridin-3-yl)piperazine-1-carboxylate as a yellow oil (100 mg, 44% yield). ESI-MS (M+H)⁺: 549.4.

### 10. Synthesis of (R)-5-(tert-butyl)-N-(1-(2-methyl-4-(3-(piperazin-1-yl)pyridin-4-yl)phenyl)ethyl)-1,2,4-oxadiazole-3-carboxamide hydrochloride

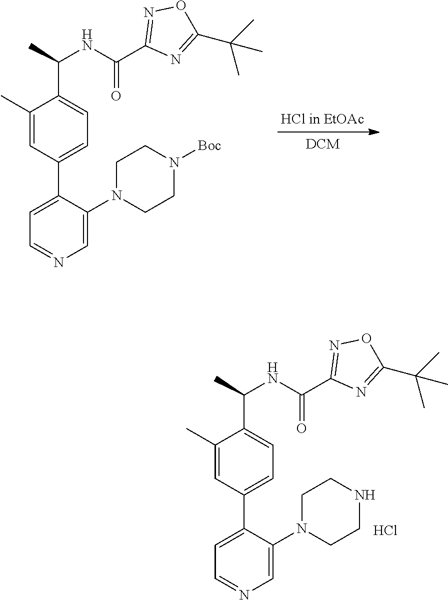 [SMILES: Cc1cc(-c2ccncc2N2CCN(C(=O)OC(C)(C)C)CC2)ccc1[C@@H](C)NC(=O)c1noc(C(C)(C)C)n1 ; Cc1cc(-c2ccncc2N2CCNCC2)ccc1[C@@H](C)NC(=O)c1noc(C(C)(C)C)n1 ; Cl]

To a solution of tert-butyl (R)-4-(4-(4-(1-(5-(tert-butyl)-1,2,4-oxadiazole-3-carboxamido)ethyl)-3-methylphenyl)pyridin-3-yl)piperazine-1-carboxylate (100 mg, 182 μmol) in DCM (10 mL) was added a solution of HCl in ethyl acetate (8 mL, 4 M) at 20° C. The mixture was stirred at 20° C. for 1 hour. The mixture was concentrated under vacuum to give (R)-5-(tert-butyl)-N-(1-(2-methyl-4-(3-(piperazin-1-yl)pyridin-4-yl)phenyl)ethyl)-1,2,4-oxadiazole-3-carboxamide hydrochloride as a yellow solid (80 mg, crude), which was carried forward without further purification. ESI-MS (M+H)⁺: 449.3.

### 11. Synthesis of (R,E)-5-(tert-butyl)-N-(1-(4-(3-(4-(4-(dimethylamino)but-2-enoyl)piperazin-1-yl)pyridin-4-yl)-2-methylphenyl)ethyl)-1,2,4-oxadiazole-3-carboxamide

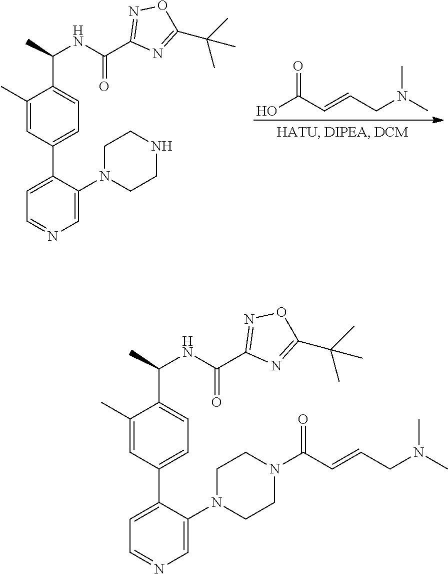

To a solution of (R)-5-(tert-butyl)-N-(1-(2-methyl-4-(3-(piperazin-1-yl)pyridin-4-yl)phenyl)ethyl)-1,2,4-oxadiazole-3-carboxamide hydrochloride (80 mg, 165 μmol) in DCM (50 mL) was added DIPEA (43 mg, 330 μmol), (E)-4-(dimethylamino)but-2-enoic acid (21 mg, 165 μmol) and HATU (63 mg, 165 μmol) at 20° C. The mixture was stirred at 20° C. for 30 minutes. The mixture was concentrated under vacuum. The residue was purified by prep-HPLC (Column: Welch Xtimate C18 150×25 mm×5 μm; Condition: water (10 mM NH₄HCO₃)-ACN, Begin B 33 End B 63, Gradient Time (min) 10, 100% B Hold Time (min) 2, Flow Rate (mL/min): 25) to give (R,E)-5-(tert-butyl)-N-(1-(4-(3-(4-(4-(dimethylamino)but-2-enoyl)piperazin-1-yl)pyridin-4-yl)-2-methylphenyl)ethyl)-1,2,4-oxadiazole-3-carboxamide as a yellow solid (37 mg, 40% yield, 100% ee). ESI-MS (M+H)⁺: 560.3. ¹H NMR: (500 MHz, DMSO-d₆) δ=9.43 (d, J=8.0 Hz, 1H), 8.32-8.26 (m, 2H), 7.59-7.52 (m, 3H), 7.23 (d, J=5.0 Hz, 1H), 6.62-6.53 (m, 2H), 5.38-5.31 (m, 1H), 3.49-3.46 (m, 4H), 2.99 (d, J=5.0 Hz, 2H), 2.84-2.81 (m, 4H), 2.43 (s, 3H), 2.11 (s, 6H), 1.48 (d, J=7.0 Hz, 3H), 1.41 (s, 9H).

### Example 87: (E)-5-(tert-butyl)-N-(2-(difluoromethyl)-4-(3-(4-(4-(dimethylamino)but-2-enoyl)piperazin-1-yl)pyridin-4-yl)benzyl)-1,2,4-oxadiazole-3-carboxamide

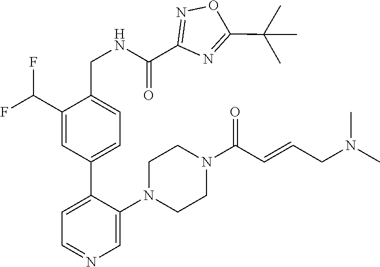 [SMILES: CN(C)C/C=C/C(=O)N1CCN(c2cnccc2-c2ccc(CNC(=O)c3noc(C(C)(C)C)n3)c(C(F)F)c2)CC1]

### 1. Synthesis of 4-bromo-2-(dibromomethyl)benzonitrile

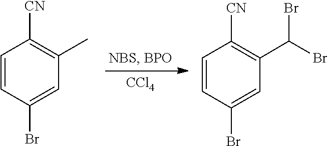

To a solution of 4-bromo-2-methylbenzonitrile (15 g, 76.5 mmol) in CCl₄ (500 mL) was added NBS (41 g, 229 mmol) and BPO (1.9 g, 7.65 mmol). The mixture was refluxed for 64 h. The solid was filtered off and washed with EtOAc (500 mL). The filtrate was concentrated under vacuum to give the crude, which was purified by chromatography column on silica gel (petroleum ether/ethyl acetate=40/1) to give 4-bromo-2-(dibromomethyl)benzonitrile as a white solid (28 g, 100% yield). ¹H NMR: (400 MHz, CDCl₃) δ: 8.16 (s, 1H), 7.60-7.57 (m, 1H), 7.48-7.46 (d, 1H), 6.90 (s, 1H).

### 2. Synthesis of 4-bromo-2-formylbenzonitrile

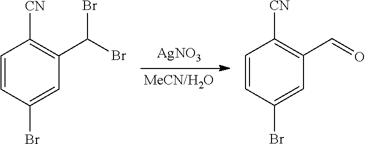

To a solution of 4-bromo-2-(dibromomethyl)benzonitrile (28 g, 79.3 mmol, 1.0 eq.) in MeCN (105 mL) was added a solution of AgNO₃ (54 g, 317 mmol) in H₂O (35 mL). The mixture was stirred at 90° C. for 30 minutes. The mixture was filtered, and the filter cake was washed with DCM several times. The filtrate was washed with brine (200 mL), dried over Na₂SO₄, filtered, and concentrated under vacuum to give crude material. The crude was purified by chromatography column on silica gel (petroleum ether/ethyl acetate=40/1 to 5/1) to give 4-bromo-2-formylbenzonitrile as a white solid (11 g, yield: 66%). ¹H NMR: (400 MHz, CDCl₃) δ: 10.29 (s, 1H), 8.16 (d, J=2.0 Hz, 1H), 7.87 (dd, J=8.4 Hz, 2.0 Hz, 1H), 7.68 (d, J=8.0 Hz, 1H).

### 3. Synthesis of 4-bromo-2-(difluoromethyl)benzonitrile

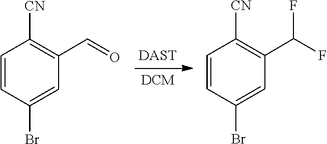

To a solution of 4-bromo-2-formylbenzonitrile (11 g, 52.4 mmol) in DCM (10 mL) was added a solution of DAST (21 g, 131 mmol) in DCM (100 mL) at ice-bath slowly. The mixture was stirred at 26° C. for 30 minutes. The mixture concentrated under vacuum to give the crude, which was purified by chromatography column on silica gel (petroleum ether/ethyl acetate=20/1 to 5/1) to give the 4-bromo-2-(difluoromethyl)benzonitrile as a yellow solid (10.4 g, yield: 86%). ¹H NMR: (400 MHz, CDCl₃) δ: 7.92 (s, 1H), 7.75 (dd, J=8.0 Hz, 0.8 Hz, 1H), 7.62 (d, J=8.0 Hz, 1H), 6.89 (t, J=54.4 Hz, 1H).

### 4. Synthesis of tert-butyl (4-bromo-2-(difluoromethyl)benzyl)carbamate

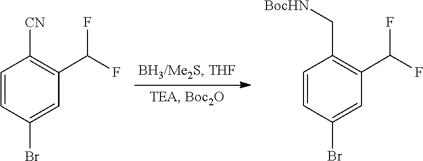

To a solution of 4-bromo-2-(difluoromethyl)benzonitrile (10.4 g, 44.8 mmol) in THF (100 mL) was added BH₃/Me₂S (13.4 mL, 134 mmol). The mixture was stirred at 80° C. for 18 h. The mixture was quenched with MeOH (100 mL), then Boc₂O (19.5 g, 89.6 mmol) and triethylamine (18 g, 179 mmol) was added. The mixture was stirred at 26° C. for 3 h. The mixture was concentrated under vacuum to give the crude, which was purified by chromatography column on silica gel (petroleum ether/ethyl acetate=50/1 to 20/1) to give tert-butyl (4-bromo-2-(difluoromethyl)benzyl)carbamate as a yellow oil (8.6 g, yield: 57%, two steps). ¹H NMR: (400 MHz, CDCl₃) δ: 7.65 (s, 1H), 7.55 (d, J=8.0 Hz, 1H), 7.30 (d, J=8.4 Hz, 1H), 6.70 (t, J=54.8 Hz, 1H), 4.90 (br, 1H), 4.36 (d, J=6.0 Hz, 2H), 1.42 (s, 9H).

### 5. Synthesis of tert-butyl (2-(difluoromethyl)-4-(4,4,5,5-tetramethyl-1,3,2-dioxaborolan-2-yl)benzyl)carbamate

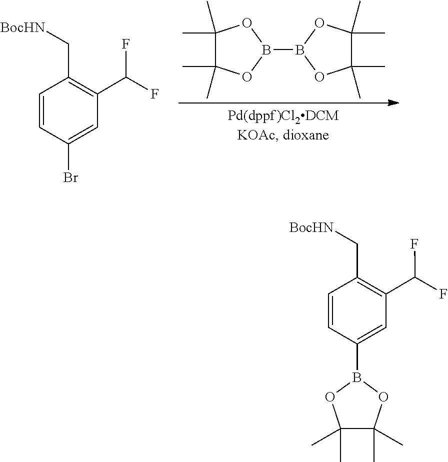

To a solution of tert-butyl (4-bromo-2-(difluoromethyl)benzyl)carbamate (3 g, 8.9 mmol) in dioxane (40 mL) was added KOAc (1.75 g, 17.8 mmol) and bis(pinacolato)diboron (2.5 g, 9.8 mmol). Then Pd(dppf)Cl₂•DCM (363 mg, 0.45 mmol) was added under an N₂ atmosphere. The mixture was stirred at 85° C. for 3 h. The mixture concentrated under vacuum to give the crude, which was purified by chromatography column on silica gel (petroleum ether/ethyl acetate=10/1 to 5/1) to give the tert-butyl (2-(difluoromethyl)-4-(4,4,5,5-tetramethyl-1,3,2-dioxaborolan-2-yl)benzyl)carbamate as a yellow oil (3.2 g, yield: 94%). ¹H NMR: (400 MHz, CDCl₃) δ: 7.90-7.79 (m, 2H), 7.44 (d, J=7.6 Hz, 1H), 6.79 (t, J=55.2 Hz, 1H), 4.89 (br, 1H), 4.46 (d, J=5.6 Hz, 2H), 1.42 (s, 9H), 1.35-1.32 (m, 12H).

### 6. Synthesis of 1-(4-chloropyridin-3-yl)piperazine hydrochloride

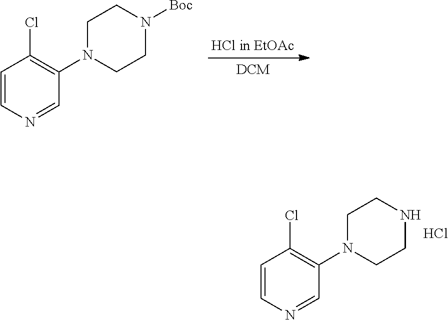

To a solution of tert-butyl 4-(4-chloropyridin-3-yl)piperazine-1-carboxylate (1 g, 3.36 mmol) in DCM (15 mL) was added a solution of HCl in ethyl acetate (4 M, 20 mL) and the mixture was stirred at 20° C. for 1 hour. The mixture was concentrated under vacuum to give crude 1-(4-chloropyridin-3-yl)piperazine hydrochloride as a yellow solid (0.78 g, yield: 100%), which was carried forward without further purification. LCMS: m/z=198.0 (M+H⁺).

### 7. Synthesis of (E)-1-(4-(4-chloropyridin-3-yl)piperazin-1-yl)-4-(dimethylamino)but-2-en-1-one

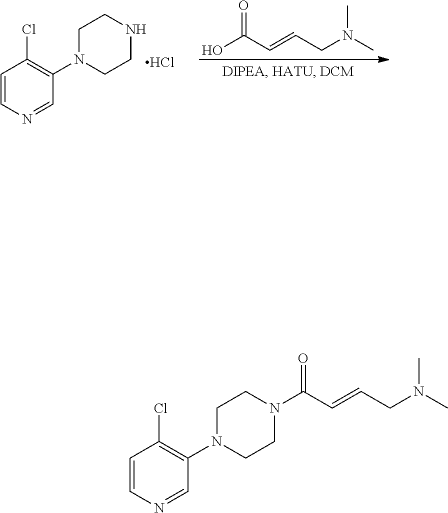

To a solution of 1-(4-chloropyridin-3-yl)piperazine hydrochloride (0.74 g, 3.16 mmol) in DCM (80 mL) was added DIPEA (1.23 g, 9.48 mmol, 1.65 mL). Then (E)-4-(dimethylamino)but-2-enoic acid (490 mg, 3.79 mmol) and HATU (1.20 g, 3.16 mmol) were added into the mixture and the mixture was stirred at 20° C. for 2 hours. The reaction mixture was concentrated under vacuum and purified by Prep-HPLC (Column: Welch Xtimate C18 150×25 mm×5 μm; Condition: water (10 mM NH4HCO3)-ACN, Begin B 18, End B 42, Gradient Time (min) 10, 100% B Hold Time (min) 2, Flow Rate (mL/min) 25.) to give (E)-1-(4-(4-chloropyridin-3-yl)piperazin-1-yl)-4-(dimethylamino)but-2-en-1-one as a white solid (0.7 g, 69% yield). LCMS: m/z=309.1 (M+H⁺).

### 8. Synthesis of tert-butyl (E)-(2-(difluoromethyl)-4-(3-(4-(4-(dimethylamino)but-2-enoyl)piperazin-1-yl)pyridin-4-yl)benzyl)carbamate

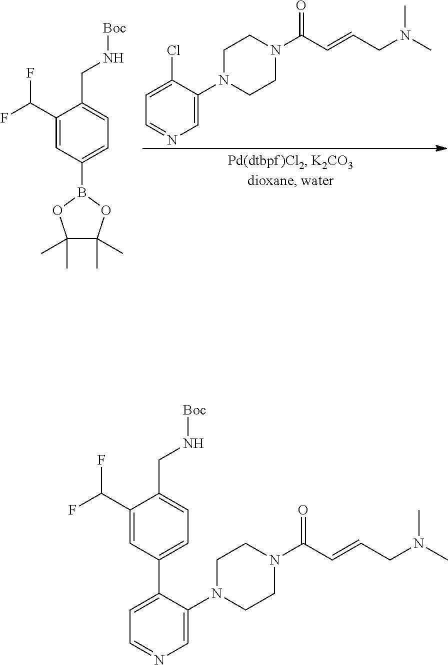

To a solution of tert-butyl (2-(difluoromethyl)-4-(4,4,5,5-tetramethyl-1,3,2-dioxaborolan-2-yl)benzyl)carbamate (0.2 g, 522 μmol) and (E)-1-(4-(4-chloropyridin-3-yl)piperazin-1-yl)-4-(dimethylamino)but-2-en-1-one (161 mg, 522 μmol) in dioxane (15 mL) and water (1.5 mL) was added K₂CO₃ (216 mg, 1.57 mmol). Then Pd(dtbpf)Cl₂ (34 mg, 52 μmol) was added into the mixture and the mixture was stirred at 90° C. under N₂ for 4 hours. The reaction mixture was concentrated under vacuum to give a crude, which was purified by column chromatography (DCM/MeOH=10:1) to give tert-butyl (E)-(2-(difluoromethyl)-4-(3-(4-(4-(dimethylamino)but-2-enoyl)piperazin-1-yl)pyridin-4-yl)benzyl)carbamate as a brown solid (0.14 g, 51% yield). LCMS: m/z=530.3 (M+H⁺).

### 9. Synthesis of (E)-1-(4-(4-(4-(aminomethyl)-3-(difluoromethyl)phenyl)pyridin-3-yl)piperazin-1-yl)-4-(dimethylamino)but-2-en-1-one

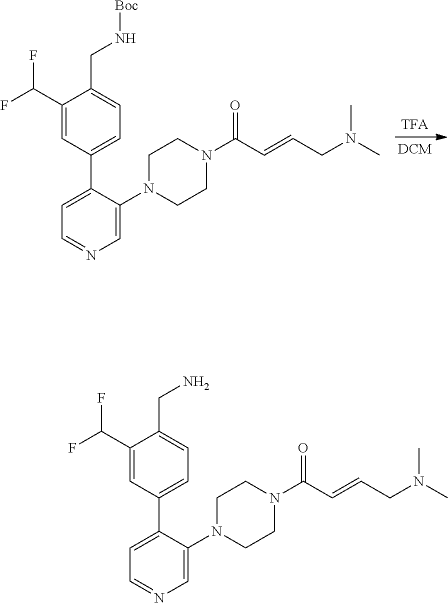 [SMILES: CN(C)C/C=C/C(=O)N1CCN(c2cnccc2-c2ccc(CNC(=O)OC(C)(C)C)c(C(F)F)c2)CC1 ; CN(C)C/C=C/C(=O)N1CCN(c2cnccc2-c2ccc(CN)c(C(F)F)c2)CC1]

To a solution of tert-butyl (E)-(2-(difluoromethyl)-4-(3-(4-(4-(dimethylamino)but-2-enoyl)piperazin-1-yl)pyridin-4-yl)benzyl)carbamate (0.14 g, 264 μmol) in DCM (10 mL) was added TFA (1.49 g, 13 mmol, 1 mL) and the mixture was stirred at 20° C. for 1 hour. The mixture was concentrated under vacuum to give (E)-1-(4-(4-(4-(aminomethyl)-3-(difluoromethyl)phenyl)pyridin-3-yl)piperazin-1-yl)-4-(dimethylamino)but-2-en-1-one as a brown oil (0.11 g, crude), which was carried forward without further purification. LCMS: m/z=430.2 (M+H⁺).

### 10. Synthesis of (E)-5-(tert-butyl)-N-(2-(difluoromethyl)-4-(3-(4-(4-(dimethylamino)but-2-enoyl)piperazin-1-yl)pyridin-4-yl)benzyl)-1,2,4-oxadiazole-3-carboxamide

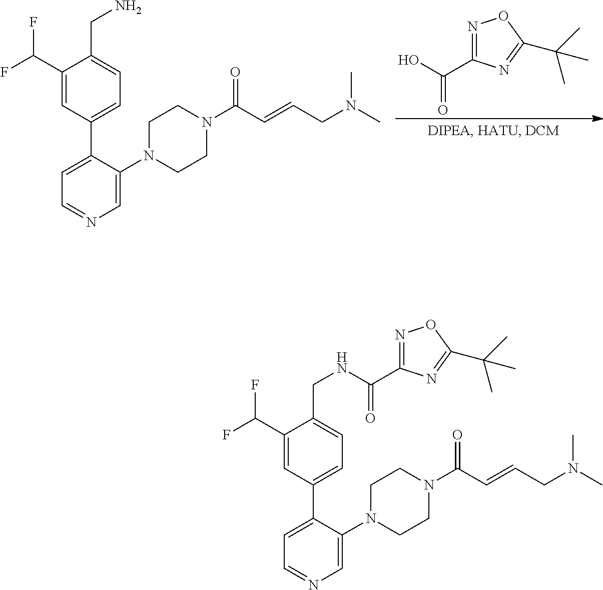 [SMILES: CN(C)C/C=C/C(=O)N1CCN(c2cnccc2-c2ccc(CN)c(C(F)F)c2)CC1 ; CC(C)(C)c1nc(C(=O)O)no1 ; CN(C)C/C=C/C(=O)N1CCN(c2cnccc2-c2ccc(CNC(=O)c3noc(C(C)(C)C)n3)c(C(F)F)c2)CC1]

To a solution of (E)-1-(4-(4-(4-(aminomethyl)-3-(difluoromethyl)phenyl)pyridin-3-yl)piperazin-1-yl)-4-(dimethylamino)but-2-en-1-one (90 mg, 210 μmol) in DCM (50 mL) was added DIPEA (81 mg, 629 μmol, 110 μL). Then 5-(tert-butyl)-1,2,4-oxadiazole-3-carboxylic acid (53 mg, 314 mol) and HATU (80 mg, 210 μmol) was added into the mixture and the mixture was stirred at 20° C. for 1 hour. The reaction mixture was concentrated under vacuum to give a crude, which was purified by Prep-HPLC (Column: Welch Xtimate C18 150×25 mm×5 μm; Condition: water (10 mM NH₄HCO₃)-ACN, Begin B 35, End B 65, Gradient Time (min) 10, 100% B Hold Time (min) 2, Flow Rate (mL/min) 25.) to give (E)-5-(tert-butyl)-N-(2-(difluoromethyl)-4-(3-(4-(4-(dimethylamino)but-2-enoyl)piperazin-1-yl)pyridin-4-yl)benzyl)-1,2,4-oxadiazole-3-carboxamide as a white solid (47 mg, 38% yield). LCMS: m/z=582.4 (M+H⁺). ¹HNMR: (400 MHz, DMSO-d₆) δ=9.57 (t, J=5.6 Hz, 1H), 8.34 (d, J=11.2 Hz, 2H), 8.09 (s, 1H), 7.87 (d, J=8.4 Hz, 1H), 7.55 (d, J=8.0 Hz, 1H), 7.40-7.26 (m, 2H), 6.57 (s, 2H), 4.67 (d, J=5.6 Hz, 2H), 3.46 (s, 4H), 3.00 (s, 2H), 2.83 (s, 4H), 2.12 (s, 6H), 1.43 (s, 9H).

### Example 88: (E)-N-(2-(difluoromethyl)-4-(3-(4-(4-(dimethylamino)but-2-enoyl)piperazin-1-yl)pyridin-4-yl)benzyl)-5-(1-methylcyclopropyl)-1,2,4-oxadiazole-3-carboxamide

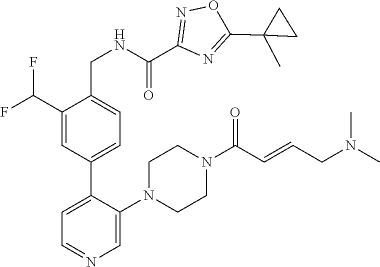 [SMILES: CN(C)C/C=C/C(=O)N1CCN(c2cnccc2-c2ccc(CNC(=O)c3noc(C4(C)CC4)n3)c(C(F)F)c2)CC1]

### 1. Synthesis of N-(2-(difluoromethyl)-4-(4,4,5,5-tetramethyl-1,3,2-dioxaborolan-2-yl)benzyl)-5-(1-methylcyclopropyl)-1,2,4-oxadiazole-3-carboxamide

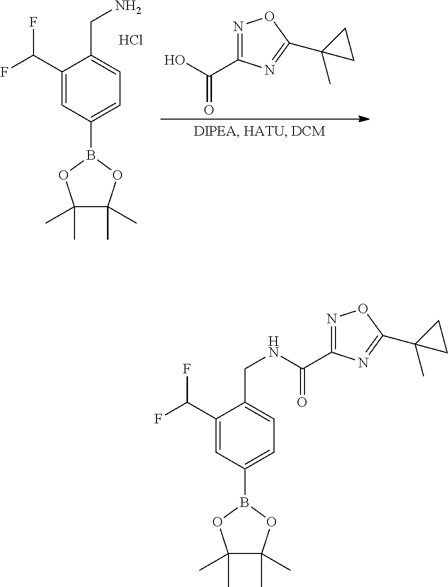 [SMILES: CC1(C)OB(c2ccc(CN)c(C(F)F)c2)OC1(C)C ; Cl ; CC1(c2nc(C(=O)O)no2)CC1 ; CC1(c2nc(C(=O)NCc3ccc(B4OC(C)(C)C(C)(C)O4)cc3C(F)F)no2)CC1]

2. Synthesis of N-(2-(difluoromethyl)-4-(4,4,5,5-tetramethyl-1,3,2-dioxaborolan-2-yl)benzyl)-5-(1-methylcyclopropyl)-1,2,4-oxadiazole-3-carboxamide was similar to that of N-(2-methyl-4-(4,4,5,5-tetramethyl-1,3,2-dioxaborolan-2-yl)benzyl)-3-(1-methylcyclopropyl)-1,2,4-oxadiazole-5-carboxamide in Example 73, Step 1. The crude material was purified by silica gel chromatography (grading from petroleum ether to petroleum ether/ethyl acetate=3/1) to give N-(2-(difluoromethyl)-4-(4,4,5,5-tetramethyl-1,3,2-dioxaborolan-2-yl)benzyl)-5-(1-methylcyclopropyl)-1,2,4-oxadiazole-3-carboxamide as a yellow oil (200 mg, 33% yield). LCMS: m/z=434.3 (M+H⁺).

### 3. Synthesis of tert-butyl 4-(4-(3-(difluoromethyl)-4-((5-(1-methylcyclopropyl)-1,2,4-oxadiazole-3-carboxamido)methyl)phenyl)pyridin-3-yl)piperazine-1-carboxylate

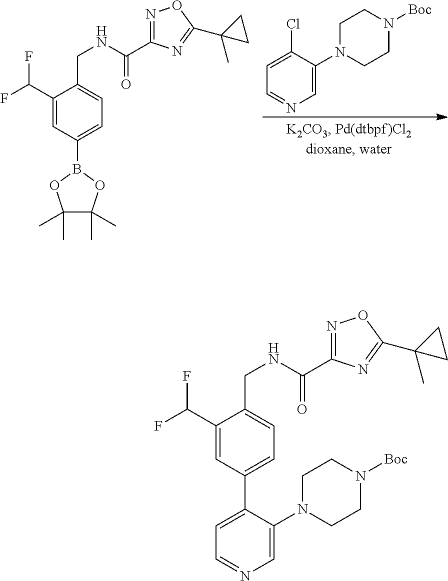

To a solution of N-(2-(difluoromethyl)-4-(4,4,5,5-tetramethyl-1,3,2-dioxaborolan-2-yl)benzyl)-5-(1-methylcyclopropyl)-1,2,4-oxadiazole-3-carboxamide (200 mg, 462 μmol) in dioxane (5 mL) and water (1 mL) was added K₂CO₃ (128 mg, 923 μmol), tert-butyl 4-(4-chloropyridin-3-yl)piperazine-1-carboxylate (137 mg, 462 μmol) and Pd(dtbpf)Cl₂ (30 mg, 46 μmol) at 20° C. The mixture was stirred at 90° C. under N₂ for 2 hours. The mixture was concentrated under vacuum to give a residue. The residue was purified by silica gel chromatography (from petroleum ether to ethyl acetate) to give tert-butyl 4-(4-(3-(difluoromethyl)-4-((5-(1-methylcyclopropyl)-1,2,4-oxadiazole-3-carboxamido)methyl)phenyl)pyridin-3-yl)piperazine-1-carboxylate as a yellow oil (100 mg, 38% yield). LCMS: m/z=569.5 (M+H⁺).

### 4. Synthesis of N-(2-(difluoromethyl)-4-(3-(piperazin-1-yl)pyridin-4-yl)benzyl)-5-(1-methylcyclopropyl)-1,2,4-oxadiazole-3-carboxamide hydrochloride

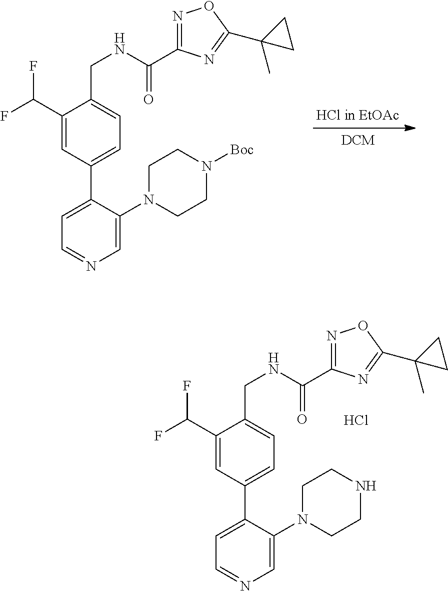 [SMILES: CC(C)(C)OC(=O)N1CCN(c2cnccc2-c2ccc(CNC(=O)c3noc(C4(C)CC4)n3)c(C(F)F)c2)CC1 ; CC1(c2nc(C(=O)NCc3ccc(-c4ccncc4N4CCNCC4)cc3C(F)F)no2)CC1 ; Cl]

To a solution of tert-butyl 4-(4-(3-(difluoromethyl)-4-((5-(1-methylcyclopropyl)-1,2,4-oxadiazole-3-carboxamido)methyl)phenyl)pyridin-3-yl)piperazine-1-carboxylate (100 mg, 176 μmol) in DCM (10 mL) was added an HCl solution in ethyl acetate (8 mL, 4 M) at 20° C. The mixture was stirred at 20° C. for 1 hour. The mixture was concentrated under vacuum to give N-(2-(difluoromethyl)-4-(3-(piperazin-1-yl)pyridin-4-yl)benzyl)-5-(1-methylcyclopropyl)-1,2,4-oxadiazole-3-carboxamide hydrochloride as a yellow solid (80 mg, crude), which was carried forward without further purification. LCMS: m/z=469.2 (M+H⁺).

### 5. Synthesis of (E)-N-(2-(difluoromethyl)-4-(3-(4-(4-(dimethylamino)but-2-enoyl)piperazin-1-yl)pyridin-4-yl)benzyl)-5-(1-methylcyclopropyl)-1,2,4-oxadiazole-3-carboxamide

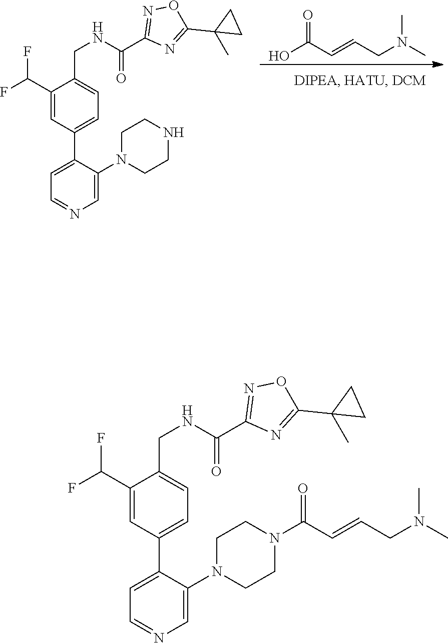 [0944] 6. Synthesis of (E)-N-(2-(difluoromethyl)-4-(3-(4-(4-(dimethylamino)but-2-enoyl)piperazin-1-yl)pyridin-4-yl)benzyl)-5-(1-methylcyclopropyl)-1,2,4-oxadiazole-3-carboxamide was similar to that of (R,E)-5-(tert-butyl)-N-(1-(4-(3-(4-(4-(dimethylamino)but-2-enoyl)piperazin-1-yl)pyridin-4-yl)-2-methylphenyl)ethyl)-1,2,4-oxadiazole-3-carboxamide in Example 86, Step 11. The crude material was purified by prep-HPLC (Column: Welch Xtimate C18 150×25 mm×5 μm; Condition: water (10 mM NH₄HCO₃)-ACN, Begin B 28 End B 58, Gradient Time (min) 10, 100% B Hold Time (min) 2, Flow Rate (mL/min): 25) to give (E)-N-(2-(difluoromethyl)-4-(3-(4-(4-(dimethylamino)but-2-enoyl)piperazin-1-yl)pyridin-4-yl)benzyl)-5-(1-methylcyclopropyl)-1,2,4-oxadiazole-3-carboxamide as a yellow solid (31 mg, 34% yield). LCMS: m/z=580.3 (M+H⁺). ¹H NMR: (500 MHz, DMSO-d₆) δ=9.52 (t, J=6.0 Hz, 1H), 8.36-8.32 (m, 2H), 8.09 (s, 1H), 7.87 (d, J=8.0 Hz, 1H), 7.57-7.37 (m, 2H), 7.29 (d, J=5.0 Hz, 1H), 6.62-6.54 (m, 2H), 4.65 (d, J=6.0 Hz, 2H), 3.49-3.46 (m, 4H), 2.99 (d, J=4.5 Hz, 2H), 2.85-2.81 (m, 4H), 2.12 (s, 6H), 1.54 (s, 3H), 1.40-1.37 (m, 2H), 1.19-1.15 (m, 2H).

### Example 89: (E)-3-(tert-butyl)-N-(2-(difluoromethyl)-4-(3-(4-(4-(dimethylamino)but-2-enoyl)piperazin-1-yl)pyridin-4-yl)benzyl)-1,2,4-oxadiazole-5-carboxamide

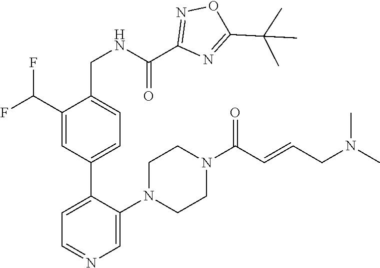 [SMILES: CN(C)C/C=C/C(=O)N1CCN(c2cnccc2-c2ccc(CNC(=O)c3noc(C(C)(C)C)n3)c(C(F)F)c2)CC1]

### 1. Synthesis of (2-(difluoromethyl)-4-(4,4,5,5-tetramethyl-1,3,2-dioxaborolan-2-yl)phenyl)methanamine hydrochloride

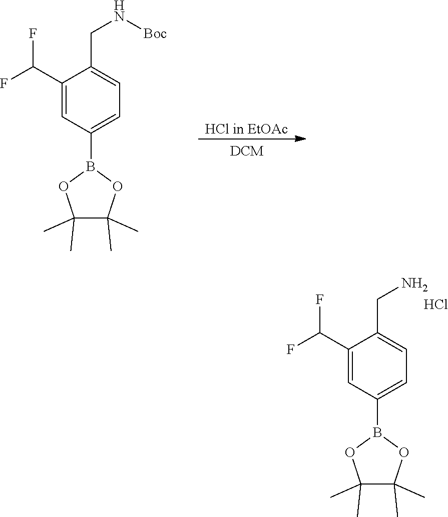

To a solution of tert-butyl (2-(difluoromethyl)-4-(4,4,5,5-tetramethyl-1,3,2-dioxaborolan-2-yl)benzyl)carbamate (1 g, 2.61 μmol) in DCM (20 mL) was added a solution of HCl in ethyl acetate (4 M, 10 mL). The mixture was stirred at 25° C. for 1 hour. The mixture was concentrated in vacuo to give (2-(difluoromethyl)-4-(4,4,5,5-tetramethyl-1,3,2-dioxaborolan-2-yl)phenyl)methanamine hydrochloride as a brown oil (700 mg, crude), which was carried forward without further purification. LCMS: m/z=284.1 (M+H⁺).

### 2. Synthesis of 3-(tert-butyl)-N-(2-(difluoromethyl)-4-(4,4,5,5-tetramethyl-1,3,2-dioxaborolan-2-yl)benzyl)-1,2,4-oxadiazole-5-carboxamide

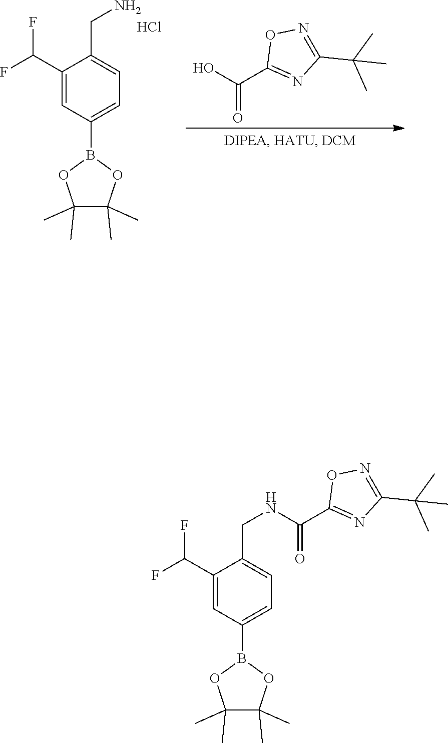 [SMILES: CC1(C)OB(c2ccc(CN)c(C(F)F)c2)OC1(C)C ; Cl ; CC(C)(C)c1noc(C(=O)O)n1 ; "DIPEA, HATU, DCM" ; CC(C)(C)c1noc(C(=O)NCc2ccc(B3OC(C)(C)C(C)(C)O3)cc2C(F)F)n1]

To a solution of (2-(difluoromethyl)-4-(4,4,5,5-tetramethyl-1,3,2-dioxaborolan-2-yl)phenyl)methanamine hydrochloride (350 mg, 1.10 mmol) in DCM (40 mL) was added DIPEA (283 mg, 2.19 mmol). Then slowly was added 3-(tert-butyl)-1,2,4-oxadiazole-5-carboxylic acid (280 mg, 1.64 mmol) and HATU (459 mg, 1.20 mmol). Then the mixture was stirred at 20° C. for 30 minutes. The mixture was concentrated under vacuum to give the crude, which was purified by silica gel column chromatography (petroleum ether/ethyl acetate=5/1) to give 3-(tert-butyl)-N-(2-(difluoromethyl)-4-(4,4,5,5-tetramethyl-1,3,2-dioxaborolan-2-yl)benzyl)-1,2,4-oxadiazole-5-carboxamide as a yellow oil (200 mg, 36% yield). LCMS: m/z=436.2 (M+H⁺).

### 3. Synthesis of tert-butyl 4-(4-(4-((3-(tert-butyl)-1,2,4-oxadiazole-5-carboxamido)methyl)-3-(difluoromethyl)phenyl)pyridin-3-yl)piperazine-1-carboxylate

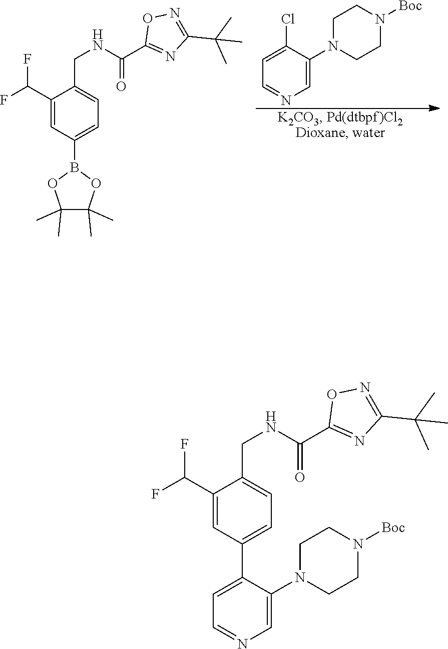

To a solution of 3-(tert-butyl)-N-(2-(difluoromethyl)-4-(4,4,5,5-tetramethyl-1,3,2-dioxaborolan-2-yl)benzyl)-1,2,4-oxadiazole-5-carboxamide (180 mg, 414 μmol) and tert-butyl 4-(4-chloropyridin-3-yl)piperazine-1-carboxylate (123 mg, 414 μmol) in dioxane (8 mL) and water (1.6 mL) was added K₂CO₃ (171 mg, 1.24 mmol). Then Pd(dtbpf)Cl₂ (40 mg, 62 μmol) was added into the mixture and the mixture was stirred at 90° C. under N₂ for 3 hours. The mixture was concentrated under vacuum to give the crude, which was purified by silica get column chromatography (1:1 petroleum ether/ethyl acetate) to give the tert-butyl 4-(4-(4-((3-(tert-butyl)-1,2,4-oxadiazole-5-carboxamido)methyl)-3-(difluoromethyl)phenyl)pyridin-3-yl)piperazine-1-carboxylate as a yellow oil (125 mg, 45% yield). LCMS: m/z=571.3 (M+H⁺).

### 4. Synthesis of 3-(tert-butyl)-N-(2-(difluoromethyl)-4-(3-(piperazin-1-yl)pyridin-4-yl)benzyl)-1,2,4-oxadiazole-5-carboxamide hydrochloride

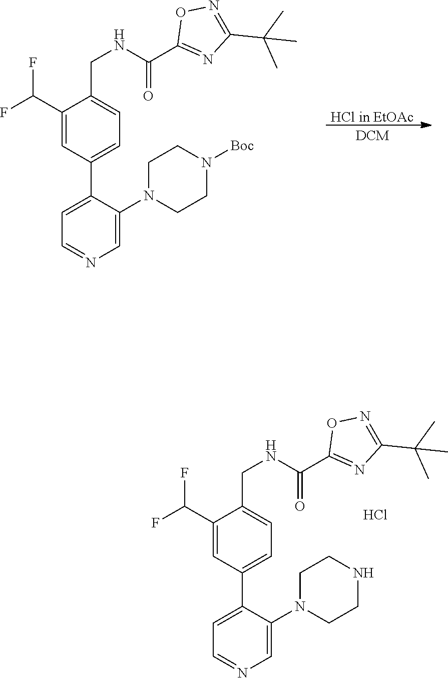

To a solution of tert-butyl 4-(4-(4-((3-(tert-butyl)-1,2,4-oxadiazole-5-carboxamido)methyl)-3-(difluoromethyl)phenyl)pyridin-3-yl)piperazine-1-carboxylate (125 mg, 219 μmol) in DCM (10 mL) was added an HCl solution in ethyl acetate (4 M, 5 mL). The mixture was stirred at 25° C. for 1 hour. The mixture was concentrated in vacuo to give 3-(tert-butyl)-N-(2-(difluoromethyl)-4-(3-(piperazin-1-yl)pyridin-4-yl)benzyl)-1,2,4-oxadiazole-5-carboxamide hydrochloride as a brown oil (100 mg, crude), which was carried forward without further purification. LCMS: m/z=471.3 (M+H⁺).

### 5. Synthesis of(E)-3-(tert-butyl)-N-(2-(difluoromethyl)-4-(3-(4-(4-(dimethylamino)but-2-enoyl)piperazin-1-yl)pyridin-4-yl)benzyl)-1,2,4-oxadiazole-5-carboxamide

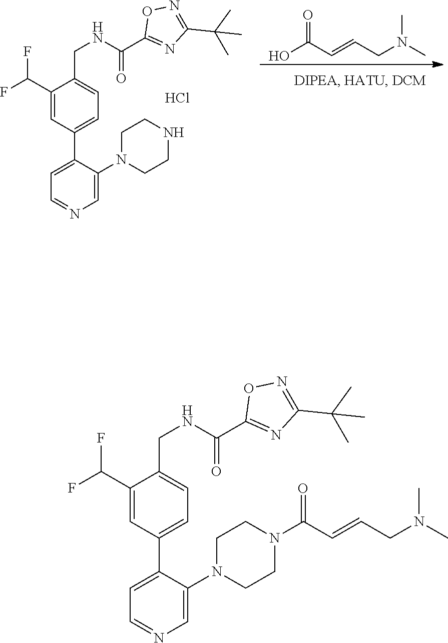 [0955] 6. Synthesis of (E)-3-(tert-butyl)-N-(2-(difluoromethyl)-4-(3-(4-(4-(dimethylamino)but-2-enoyl)piperazin-1-yl)pyridin-4-yl)benzyl)-1,2,4-oxadiazole-5-carboxamide was similar to that of (R,E)-5-(tert-butyl)-N-(1-(4-(3-(4-(4-(dimethylamino)but-2-enoyl)piperazin-1-yl)pyridin-4-yl)-2-methylphenyl)ethyl)-1,2,4-oxadiazole-3-carboxamide in Example 86, Step 11. The crude material was purified by prep-HPLC (Instrument EH; Method Column Welch Xtimate C18 150×25 mm×5 μm; Condition water (10 mM NH₄HCO₃)-ACN Begin B 31, End B 51 Gradient Time (min) 15, 100% B Hold Time (min) 2 Flow Rate (ml/min) 25, Injections 6) to give (E)-3-(tert-butyl)-N-(2-(difluoromethyl)-4-(3-(4-(4-(dimethylamino)but-2-enoyl)piperazin-1-yl)pyridin-4-yl)benzyl)-1,2,4-oxadiazole-5-carboxamide as a white solid (30 mg, 26% yield). LCMS: m/z=582.1 (M+H⁺). ¹HNMR: (500 MHz, MeOH-d₄) δ=8.35-8.27 (m, 2H), 8.08 (s, 1H), 7.87 (d, J=10.0 Hz, 1H), 7.66 (d, J=5.0 Hz, 1H), 7.40-7.34 (m, 1H), 7.34-7.09 (m, 1H), 6.77-6.72 (m, 1H), 6.58 (d, J=15.0 Hz, 1H), 4.79-4.77 (m, 2H), 3.59-3.57 (m, 4H), 3.18-3.11 (m, 2H), 2.97-2.90 (m, 4H), 2.27 (s, 6H), 1.41 (s, 9H).

### Example 90: (E)-5-(tert-butyl)-N-(4-(3-(4-(4-(dimethylamino)but-2-enoyl)piperazin-1-yl)pyridin-4-yl)-2-(trifluoromethyl)benzyl)-1,2,4-oxadiazole-3-carboxamide

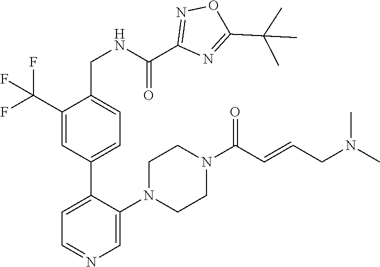 [SMILES: CN(C)C/C=C/C(=O)N1CCN(c2cnccc2-c2ccc(CNC(=O)c3noc(C(C)(C)C)n3)c(C(F)(F)F)c2)CC1]

### 1. Synthesis of (4-(4,4,5,5-tetramethyl-1,3,2-dioxaborolan-2-yl)-2-(trifluoromethyl)phenyl)methanamine hydrochloride

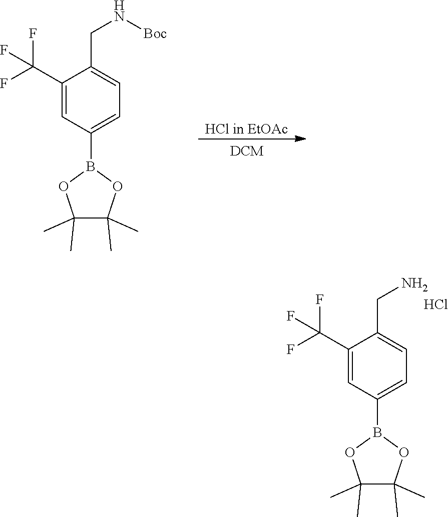

To a solution of tert-butyl (4-(4,4,5,5-tetramethyl-1,3,2-dioxaborolan-2-yl)-2-(trifluoromethyl)benzyl)carbamate (500 mg, 1.25 mmol, prepared as described in WO 2015/089337) in DCM (10 mL) was added an HCl solution in ethyl acetate (8 mL, 4 M) at 25° C. The mixture was stirred at 25° C. for 1 hour. The mixture was concentrated under vacuum to give (4-(4,4,5,5-tetramethyl-1,3,2-dioxaborolan-2-yl)-2-(trifluoromethyl)phenyl)methanamine hydrochloride as white solid (400 mg, crude), which was carried forward without further purification. LCMS: m/z=302.3 (M+H⁺).
2. Synthesis of 5-(tert-butyl)-N-(4-(4,4,5,5-tetramethyl-1,3,2-dioxaborolan-2-yl)-2-(trifluoromethyl)benzyl)-1,2,4-oxadiazole-3-carboxamide

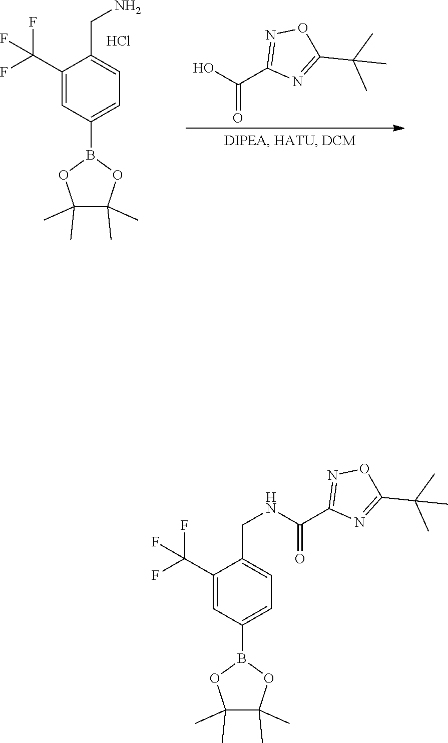 [SMILES: CC1(C)OB(c2ccc(CN)c(C(F)(F)F)c2)OC1(C)C ; Cl ; CC(C)(C)c1nc(C(=O)O)no1 ; "DIPEA, HATU, DCM" ; CC(C)(C)c1nc(C(=O)NCc2ccc(B3OC(C)(C)C(C)(C)O3)cc2C(F)(F)F)no1]

3. Synthesis of 5-(tert-butyl)-N-(4-(4,4,5,5-tetramethyl-1,3,2-dioxaborolan-2-yl)-2-(trifluoromethyl)benzyl)-1,2,4-oxadiazole-3-carboxamide was similar to that of 3-(tert-butyl)-N-(2-(difluoromethyl)-4-(4,4,5,5-tetramethyl-1,3,2-dioxaborolan-2-yl)benzyl)-1,2,4-oxadiazole-5-carboxamide in Example 89, Step 2. The crude material was purified by silica gel column chromatography (grading from 0% to 25% ethyl acetate in petroleum ether) to give 5-(tert-butyl)-N-(4-(4,4,5,5-tetramethyl-1,3,2-dioxaborolan-2-yl)-2-(trifluoromethyl)benzyl)-1,2,4-oxadiazole-3-carboxamide as a yellow oil (200 mg, 38% yield). LCMS: m/z=454.2 (M+H⁺).
4. Synthesis of tert-butyl 4-(4-(4-((5-(tert-butyl)-1,2,4-oxadiazole-3-carboxamido)methyl)-3-(trifluoromethyl)phenyl)pyridin-3-yl)piperazine-1-carboxylate

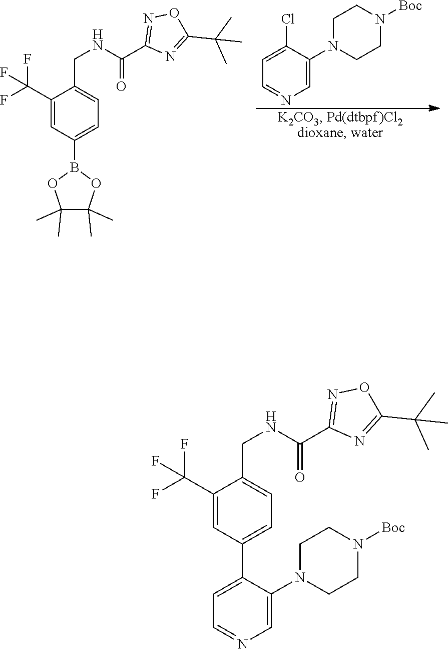

5. Synthesis of tert-butyl 4-(4-(4-((5-(tert-butyl)-1,2,4-oxadiazole-3-carboxamido)methyl)-3-(trifluoromethyl)phenyl)pyridin-3-yl)piperazine-1-carboxylate was similar to that of tert-butyl 4-(4-(4-((3-(tert-butyl)-1,2,4-oxadiazole-5-carboxamido)methyl)-3-(difluoromethyl)phenyl)pyridin-3-yl)piperazine-1-carboxylate in Example 89, Step 3. The crude material was purified by silica gel column chromatography (grading from petroleum ether to ethyl acetate) to give tert-butyl 4-(4-(4-((5-(tert-butyl)-1,2,4-oxadiazole-3-carboxamido)methyl)-3-(trifluoromethyl)phenyl)pyridin-3-yl)piperazine-1-carboxylate as a yellow oil (130 mg, 59% yield). LCMS: m/z=589.3 (M+H⁺).

### 6. Synthesis of 5-(tert-butyl)-N-(4-(3-(piperazin-1-yl)pyridin-4-yl)-2-(trifluoromethyl)benzyl)-1,2,4-oxadiazole-3-carboxamide hydrochloride

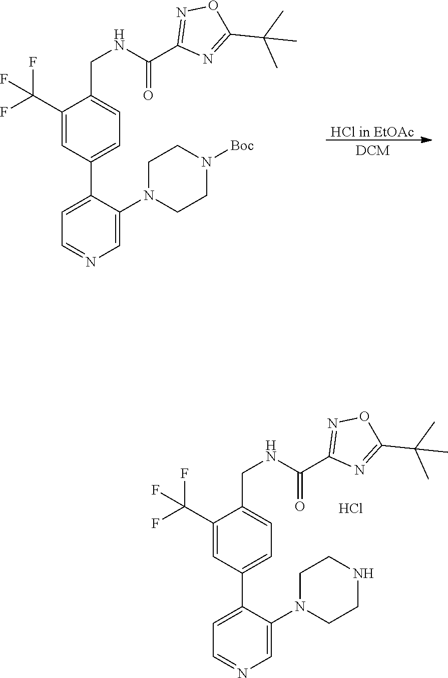

To a solution of tert-butyl 4-(4-(4-((5-(tert-butyl)-1,2,4-oxadiazole-3-carboxamido)methyl)-3-(trifluoromethyl)phenyl)pyridin-3-yl)piperazine-1-carboxylate (130 mg, 221 μmol) in DCM (15 mL) was added an HCl solution in ethyl acetate (8 mL, 4 M) at 25° C. The mixture was stirred at 25° C. for 1 hour. The mixture was concentrated under vacuum to give 5-(tert-butyl)-N-(4-(3-(piperazin-1-yl)pyridin-4-yl)-2-(trifluoromethyl)benzyl)-1,2,4-oxadiazole-3-carboxamide as a yellow solid (110 mg, crude), which was carried forward without further purification. LCMS: m/z=489.3 (M+H⁺).

### 7. Synthesis of (E)-5-(tert-butyl)-N-(4-(3-(4-(4-(dimethylamino)but-2-enoyl)piperazin-1-yl)pyridin-4-yl)-2-(trifluoromethyl)benzyl)-1,2,4-oxadiazole-3-carboxamide

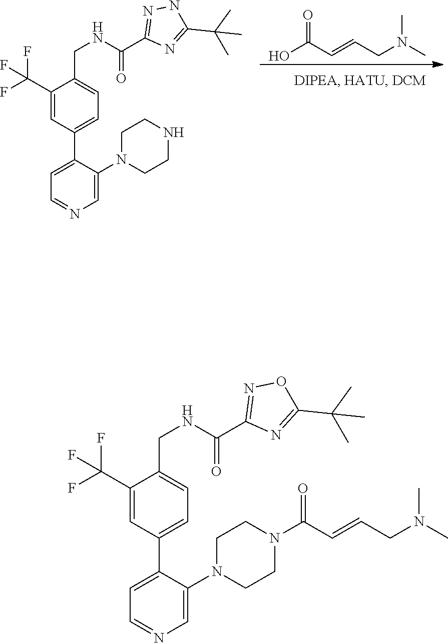 [0966] 8. Synthesis of (E)-5-(tert-butyl)-N-(4-(3-(4-(4-(dimethylamino)but-2-enoyl)piperazin-1-yl)pyridin-4-yl)-2-(trifluoromethyl)benzyl)-1,2,4-oxadiazole-3-carboxamide was similar to that of (R,E)-5-(tert-butyl)-N-(1-(4-(3-(4-(4-(dimethylamino)but-2-enoyl)piperazin-1-yl)pyridin-4-yl)-2-methylphenyl)ethyl)-1,2,4-oxadiazole-3-carboxamide in Example 86, Step 11. The crude material was purified by prep-HPLC (Column: Welch Xtimate C18 150×25 mm×5 μm; Condition: water (10 mM NH₄HCO₃)-ACN, Begin B 35 End B 65, Gradient Time (min) 10, 100% B Hold Time (min) 2, Flow Rate (mL/min): 25) to give (E)-5-(tert-butyl)-N-(4-(3-(4-(4-(dimethylamino)but-2-enoyl)piperazin-1-yl)pyridin-4-yl)-2-(trifluoromethyl)benzyl)-1,2,4-oxadiazole-3-carboxamide as a yellow solid (41 mg, 33% yield). LCMS: m/z=600.3 (M+H⁺). ¹H NMR: (500 MHz, DMSO-d₆) δ=9.62 (t, J=6.0 Hz, 1H), 8.39 (s, 1H), 8.36 (d, J=5.0 Hz, 1H), 8.21 (s, 1H), 8.02 (d, J=8.0 Hz, 1H), 7.62 (d, J=8.0 Hz, 1H), 7.34 (d, J=5.0 Hz, 1H), 6.63-6.54 (m, 2H), 4.70 (d, J=6.0 Hz, 2H), 3.48-3.45 (m, 4H), 2.99 (d, J=4.5 Hz, 2H), 2.86-2.82 (m, 4H), 2.12 (s, 6H), 1.44 (s, 9H).

### Example 91: (E)-N-(4-(3-(4-(4-(dimethylamino)but-2-enoyl)piperazin-1-yl)pyridin-4-yl)-2-(trifluoromethyl)benzyl)-5-(1-methylcyclopropyl)-1,2,4-oxadiazole-3-carboxamide

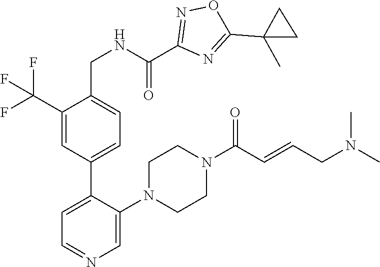 [SMILES: CN(C)C/C=C/C(=O)N1CCN(c2cnccc2-c2ccc(CNC(=O)c3noc(C4(C)CC4)n3)c(C(F)(F)F)c2)CC1] [0968] 1. Synthesis of 5-(1-methylcyclopropyl)-N-(4-(4,4,5,5-tetramethyl-1,3,2-dioxaborolan-2-yl)-2-(trifluoromethyl)benzyl)-1,2,4-oxadiazole-3-carboxamide

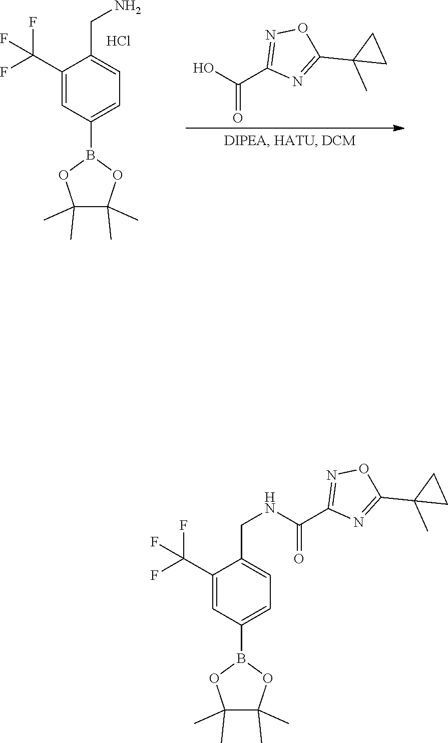 [SMILES: CC1(C)OB(c2ccc(CN)c(C(F)(F)F)c2)OC1(C)C ; Cl ; CC1(c2nc(C(=O)O)no2)CC1 ; "DIPEA, HATU, DCM" ; CC1(c2nc(C(=O)NCc3ccc(B4OC(C)(C)C(C)(C)O4)cc3C(F)(F)F)no2)CC1]

2. Synthesis of 5-(1-methylcyclopropyl)-N-(4-(4,4,5,5-tetramethyl-1,3,2-dioxaborolan-2-yl)-2-(trifluoromethyl)benzyl)-1,2,4-oxadiazole-3-carboxamide was similar to that of 5-(tert-butyl)-N-(4-(4,4,5,5-tetramethyl-1,3,2-dioxaborolan-2-yl)-2-(trifluoromethyl)benzyl)-1,2,4-oxadiazole-3-carboxamide in Example 90, Step 2. The crude material was purified by silica gel column chromatography (from petroleum ether to petroleum ether/ethyl acetate=3/1) to give 5-(1-methylcyclopropyl)-N-(4-(4,4,5,5-tetramethyl-1,3,2-dioxaborolan-2-yl)-2-(trifluoromethyl)benzyl)-1,2,4-oxadiazole-3-carboxamide as a yellow oil (300 mg, 45% yield). LCMS: m/z=452.3 (M+H⁺).
3. Synthesis of tert-butyl 4-(4-(4-((5-(1-methylcyclopropyl)-1,2,4-oxadiazole-3-carboxamido)methyl)-3-(trifluoromethyl)phenyl)pyridin-3-yl)piperazine-1-carboxylate

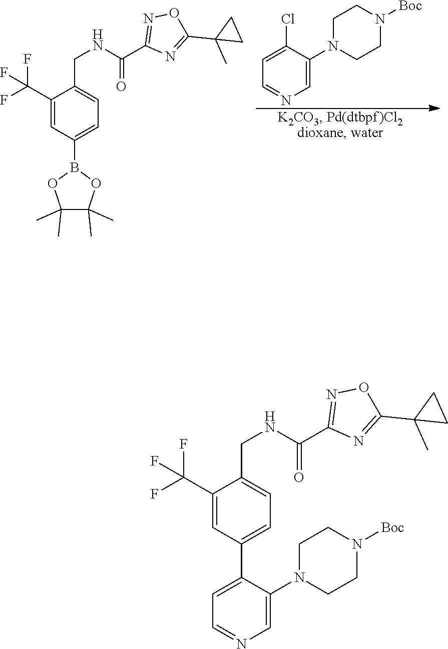

4. Synthesis of tert-butyl 4-(4-(4-((5-(1-methylcyclopropyl)-1,2,4-oxadiazole-3-carboxamido)methyl)-3-(trifluoromethyl)phenyl)pyridin-3-yl)piperazine-1-carboxylate was similar to that of tert-butyl 4-(4-(4-((3-(tert-butyl)-1,2,4-oxadiazole-5-carboxamido)methyl)-3-(difluoromethyl)phenyl)pyridin-3-yl)piperazine-1-carboxylate in Example 89, Step 3. The crude material was purified by silica gel column chromatography (from petroleum ether to ethyl acetate) to give tert-butyl 4-(4-(4-((5-(1-methylcyclopropyl)-1,2,4-oxadiazole-3-carboxamido)methyl)-3-(trifluoromethyl)phenyl)pyridin-3-yl)piperazine-1-carboxylate as a yellow oil (160 mg, 41% yield). LCMS: m/z=587.3 (M+H⁺).

### 5. Synthesis of 5-(1-methylcyclopropyl)-N-(4-(3-(piperazin-1-yl)pyridin-4-yl)-2-(trifluoromethyl)benzyl)-1,2,4-oxadiazole-3-carboxamide hydrochloride

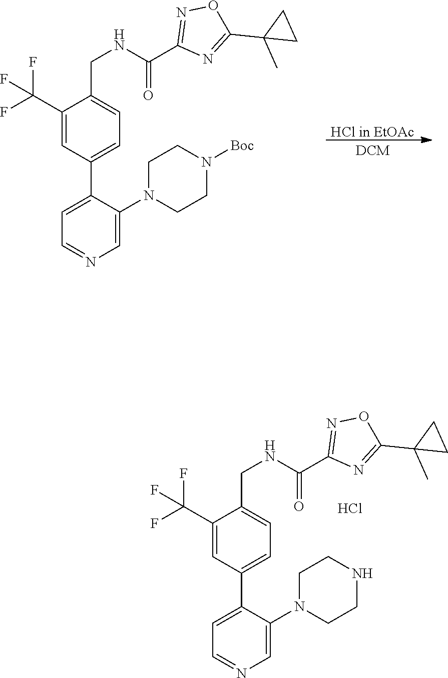

To a solution of tert-butyl 4-(4-(4-((5-(1-methylcyclopropyl)-1,2,4-oxadiazole-3-carboxamido)methyl)-3-(trifluoromethyl)phenyl)pyridin-3-yl)piperazine-1-carboxylate (160 mg, 273 μmol) in DCM (15 mL) was added an HCl solution in ethyl acetate (10 mL, 4 M) at 20° C. The mixture was stirred at 20° C. for 1 hour. The mixture was concentrated under vacuum to give 5-(1-methylcyclopropyl)-N-(4-(3-(piperazin-1-yl)pyridin-4-yl)-2-(trifluoromethyl)benzyl)-1,2,4-oxadiazole-3-carboxamide hydrochloride as a yellow solid (140 mg, crude), which was carried forward without further purification. LCMS: m/z=487.3 (M+H⁺).

### 6. Synthesis of (E)-N-(4-(3-(4-(4-(dimethylamino)but-2-enoyl)piperazin-1-yl)pyridin-4-yl)-2-(trifluoromethyl)benzyl)-5-(1-methylcyclopropyl)-1,2,4-oxadiazole-3-carboxamide

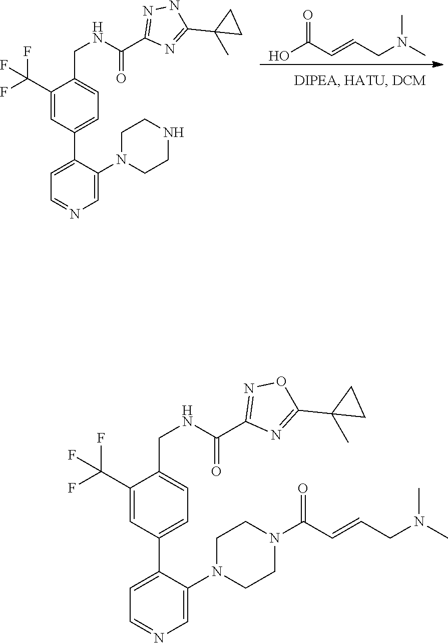 [0975] 7. Synthesis of (E)-N-(4-(3-(4-(4-(dimethylamino)but-2-enoyl)piperazin-1-yl)pyridin-4-yl)-2-(trifluoromethyl)benzyl)-5-(1-methylcyclopropyl)-1,2,4-oxadiazole-3-carboxamide was similar to that of (R,E)-5-(tert-butyl)-N-(1-(4-(3-(4-(4-(dimethylamino)but-2-enoyl)piperazin-1-yl)pyridin-4-yl)-2-methylphenyl)ethyl)-1,2,4-oxadiazole-3-carboxamide in Example 86, Step 11. The crude material was purified by prep-HPLC (Column: Welch Xtimate C18 150×25 mm×5 μm; Condition: water (10 mM NH₄HCO₃)-ACN, Begin B 34 End B 64, Gradient Time (min) 10, 100% B Hold Time (min) 2, Flow Rate (mL/min): 25) to give (E)-N-(4-(3-(4-(4-(dimethylamino)but-2-enoyl)piperazin-1-yl)pyridin-4-yl)-2-(trifluoromethyl)benzyl)-5-(1-methylcyclopropyl)-1,2,4-oxadiazole-3-carboxamide as a yellow solid (82 mg, 51% yield). LCMS: m/z=598.3 (M+H⁺). ¹H NMR: (500 MHz, DMSO-d₆) δ=9.56 (t, J=6.0 Hz, 1H), 8.40-8.32 (m, 2H), 8.20 (s, 1H), 8.01 (d, J=8.0 Hz, 1H), 7.61 (d, J=8.5 Hz, 1H), 7.34 (d, J=5.0 Hz, 1H), 6.64-6.52 (m, 2H), 4.68 (d, J=6.0 Hz, 2H), 3.46-3.43 (m, 4H), 2.99 (d, J=4.5 Hz, 2H), 2.88-2.81 (m, 4H), 2.12 (s, 6H), 1.55 (s, 3H), 1.42-1.37 (m, 2H), 1.21-1.15 (m, 2H).

### Example 92: (E)-3-(tert-butyl)-N-(4-(3-(4-(4-(dimethylamino)but-2-enoyl)piperazin-1-yl)pyridin-4-yl)-2-(trifluoromethyl)benzyl)-1,2,4-oxadiazole-5-carboxamide

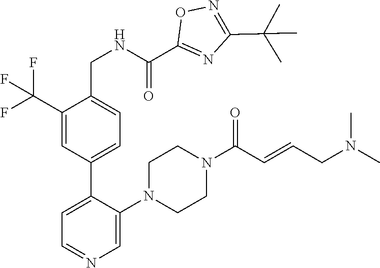 [SMILES: CN(C)C/C=C/C(=O)N1CCN(c2cnccc2-c2ccc(CNC(=O)c3nc(C(C)(C)C)no3)c(C(F)(F)F)c2)CC1]

### 1. Synthesis of 3-(tert-butyl)-N-(4-(4,4,5,5-tetramethyl-1,3,2-dioxaborolan-2-yl)-2-(trifluromethyl)benzyl)-1,2,4-oxadiazole-5-carboxamide

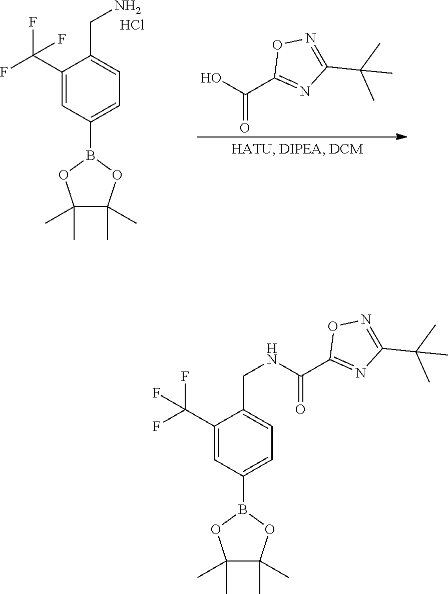 [SMILES: CC1(C)OB(c2ccc(CN)c(C(F)(F)F)c2)OC1(C)C ; Cl ; CC(C)(C)c1noc(C(=O)O)n1 ; CC(C)(C)c1noc(C(=O)NCc2ccc(B3OC(C)(C)C(C)(C)O3)cc2C(F)(F)F)n1] [0978] 2. Synthesis of 3-(tert-butyl)-N-(4-(4,4,5,5-tetramethyl-1,3,2-dioxaborolan-2-yl)-2-(trifluoromethyl)benzyl)-1,2,4-oxadiazole-5-carboxamide was similar to that of 3-(tert-butyl)-N-(2-(difluoromethyl)-4-(4,4,5,5-tetramethyl-1,3,2-dioxaborolan-2-yl)benzyl)-1,2,4-oxadiazole-5-carboxamide in Example 89, Step 2. The crude material was purified by silica gel column chromatography (ethyl acetate/petroleum ether=1/4) to give 3-(tert-butyl)-N-(4-(4,4,5,5-tetramethyl-1,3,2-dioxaborolan-2-yl)-2-(trifluoromethyl)benzyl)-1,2,4-oxadiazole-5-carboxamide as a transparent oil (430 mg, 71% yield). LCMS: m/z=454.2 (M+H⁺).
3. Synthesis of tert-butyl 4-(4-(4-((3-(tert-butyl)-1,2,4-oxadiazole-5-carboxamido)methyl)-3-(trifluoromethyl)phenyl)pyridin-3-yl)piperazine-1-carboxylate

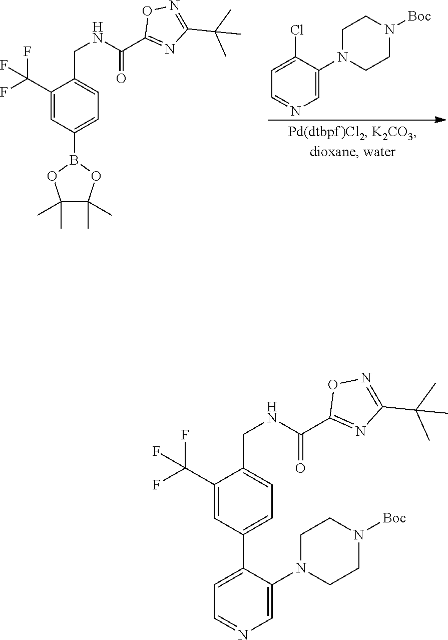 [SMILES: CC(C)(C)c1noc(C(=O)NCc2ccc(B3OC(C)(C)C(C)(C)O3)cc2C(F)(F)F)n1 ; CC(C)(C)OC(=O)N1CCN(c2cnccc2Cl)CC1 ; CC(C)(C)OC(=O)N1CCN(c2cnccc2-c2ccc(CNC(=O)c3nc(C(C)(C)C)no3)c(C(F)(F)F)c2)CC1]

4. Synthesis of tert-butyl 4-(4-(4-((3-(tert-butyl)-1,2,4-oxadiazole-5-carboxamido)methyl)-3-(trifluoromethyl)phenyl)pyridin-3-yl)piperazine-1-carboxylate was similar to that of tert-butyl 4-(4-(4-((3-(tert-butyl)-1,2,4-oxadiazole-5-carboxamido)methyl)-3-(difluoromethyl)phenyl)pyridin-3-yl)piperazine-1-carboxylate in Example 89, Step 3. The crude material was purified by silica gel column chromatography (petroleum ether/ethyl acetate=1/1) to give tert-butyl 4-(4-(4-((3-(tert-butyl)-1,2,4-oxadiazole-5-carboxamido)methyl)-3-(trifluoromethyl)phenyl)pyridin-3-yl)piperazine-1-carboxylate as a yellow oil (400 mg, 81% yield). LCMS: m/z=589.3 (M+H⁺).
5. Synthesis of 3-(tert-butyl)-N-(4-(3-(piperazin-1-yl)pyridin-4-yl)-2-(trifluoromethyl)benzyl)-1,2,4-oxadiazole-5-carboxamide hydrochloride

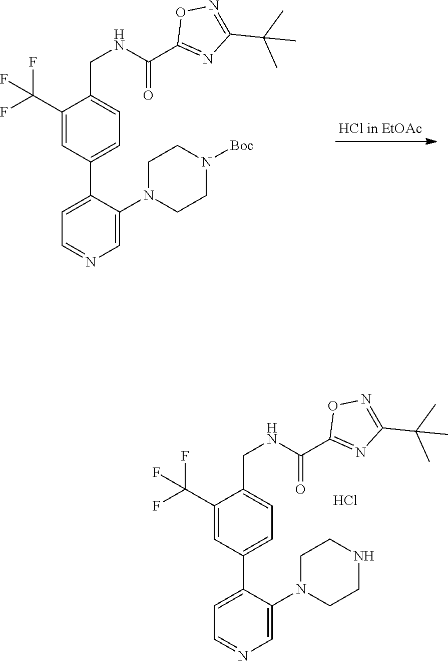 [SMILES: CC(C)(C)OC(=O)N1CCN(c2cnccc2-c2ccc(CNC(=O)c3nc(C(C)(C)C)no3)c(C(F)(F)F)c2)CC1 ; CC(C)(C)c1noc(C(=O)NCc2ccc(-c3ccncc3N3CCNCC3)cc2C(F)(F)F)n1 ; Cl]

To a solution of tert-butyl 4-(4-(4-((3-(tert-butyl)-1,2,4-oxadiazole-5-carboxamido)methyl)-3-(trifluoromethyl)phenyl)pyridin-3-yl)piperazine-1-carboxylate (400 mg, 680 μmol) in DCM (40 mL) was added an HCl solution in ethyl acetate (4 M, 8 mL). The reaction mixture was stirred at 25° C. for 2 hours. The reaction mixture was concentrated to give 3-(tert-butyl)-N-(4-(3-(piperazin-1-yl)pyridin-4-yl)-2-(trifluoromethyl)benzyl)-1,2,4-oxadiazole-5-carboxamide hydrochloride (150 mg, crude), which was carried forward without further purification. LCMS: m/z=489.2 (M+H⁺).
6. Synthesis of (E)-3-(tert-butyl)-N-(4-(3-(4-(4-(dimethylamino)but-2-enoyl)piperazin-1-yl)pyridin-4-yl)-2-(trifluoromethyl)benzyl)-1,2,4-oxadiazole-5-carboxamide

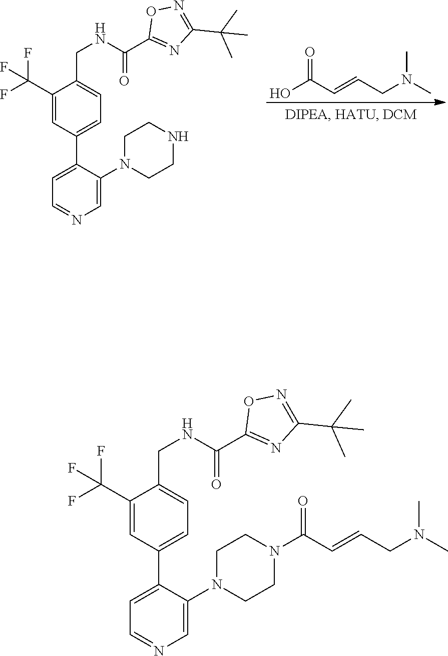

7. Synthesis of (E)-3-(tert-butyl)-N-(4-(3-(4-(4-(dimethylamino)but-2-enoyl)piperazin-1-yl)pyridin-4-yl)-2-(trifluoromethyl)benzyl)-1,2,4-oxadiazole-5-carboxamide was similar to that of (R,E)-5-(tert-butyl)-N-(1-(4-(3-(4-(4-(dimethylamino)but-2-enoyl)piperazin-1-yl)pyridin-4-yl)-2-methylphenyl)ethyl)-1,2,4-oxadiazole-3-carboxamide in Example 86, Step 11. The crude material was purified by prep-HPLC (Column: Welch Xtimate C18 150×25 mm×5 m; Condition: water (10 mM NH₄HCO₃)-ACN; Begin B 40, End B 60, Gradient Time (min) 15, 100% B Hold Time (min) 2, Flow Rate (mL/min) 25, Injections 4.) to give (E)-3-(tert-butyl)-N-(4-(3-(4-(4-(dimethylamino)but-2-enoyl)piperazin-1-yl)pyridin-4-yl)-2-(trifluoromethyl)benzyl)-1,2,4-oxadiazole-5-carboxamide as a white solid (37 mg, 20% yield). LCMS: m/z=600.4 (M+H⁺). ¹H NMR: (500 MHz, METHANOL-d₄) δ=8.36-8.34 (m, 2H), 8.20 (s, 1H), 8.01 (d, J=8.0 Hz, 1H), 7.75 (d, J=8.0 Hz, 1H), 7.39 (d, J=5.0 Hz, 1H), 6.81-6.74 (m, 1H), 6.61 (d, J=15.0 Hz, 1H), 4.87 (s, 2H), 3.59 (s, 4H), 3.17 (d, J=6.5 Hz, 2H), 2.96-2.94 (m, 4H), 2.29 (s, 6H), 1.45 (s, 9H).

### Example 93: (E)-N-(4-(3-(4-(4-(dimethylamino)but-2-enoyl)piperazin-1-yl)pyridin-4-yl)-2-methylbenzyl)-5-(1-(fluoromethyl)cyclopropyl)-1,2,4-oxadiazole-3-carboxamide

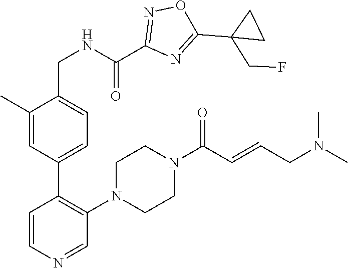

### 1. Synthesis of ethyl 1-(fluoromethyl)cyclopropane-1-carboxylate

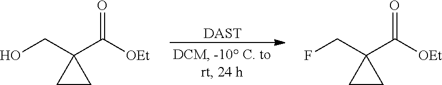

To a solution of ethyl 1-(hydroxymethyl)cyclopropane-1-carboxylate (3.0 g, 21 mmol) in DCM (50 mL) at −10° C. was added DAST (6.0 g, 40 mmol) under Ar. The reaction mixture was stirred as it warmed to ambient temperature over 1 h and continued to stir at that temperature for 23 h. An aqueous solution of HCl (10%, 5 drops) and H₂O (50 mL) were added, and the layers were separated. The aqueous layer was extracted with DCM (50 mL) and the combined organic phases were washed sequentially with H₂O (50 mL) and brine (50 mL), dried (MgSO₄), and filtered. The filtrate was concentrated in vacuo to give ethyl 1-(fluoromethyl)cyclopropane-1-carboxylate (3.0 g, 99% yield), which was carried forward without further characterization or purification.

### 2. Synthesis of 1-(fluoromethyl)cyclopropane-1-carboxylic acid

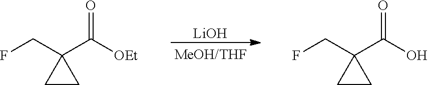 [SMILES: CCOC(=O)C1(CF)CC1 ; "LiOH" ; O=C(O)C1(CF)CC1]

To a solution of ethyl 1-(fluoromethyl)cyclopropane-1-carboxylate (5.0 g, 34 mmol) in a mixture of THF and MeOH (1:1, 50 mL) was added an aqueous solution of LiGH (2.1 g in 50 mL). The homogeneous mixture was stirred at ambient temperature for 18 h and an aqueous HCl solution (10%) was added until pH=2. The acidic aqueous phase was extracted with Et₂O (100 mL×3) and the ethereal extracts were concentrated in vacuo to give 1-(fluoromethyl)cyclopropane-1-carboxylic acid as a light brown oil (3.4 g, 85% yield), which was carried forward without further characterization or purification.

### 3. Synthesis of ethyl 5-(1-(fluoromethyl)cyclopropyl)-1,2,4-oxadiazole-3-carboxylate

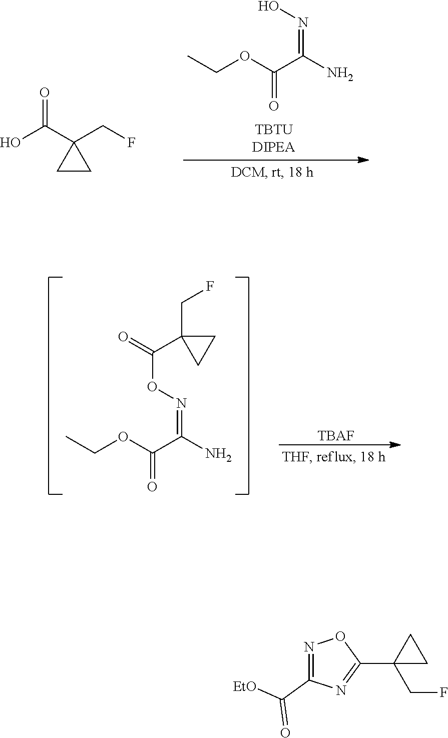

To a solution of 1-(fluoromethyl)cyclopropane-1-carboxylic acid (1.0 g, 8.5 mmol), TBTU (3.3 g, 10.2 mmol), and DIPEA (2.2 g, 16.9 mmol) in DCM (20 mL) at ambient temperature was added ethyl (E)-2-amino-2-(hydroxyimino)acetate (1.2 g, 9.3 mmol). The reaction mixture was stirred at ambient temperature for 18 h and EtOAc (100 mL) was added. The layers were separated and the organic phase was washed with a saturated aqueous NaHCO₃ solution (100 mL). The organic phase was then dried (Na₂SO₄), filtered, and concentrated in vacuo. The crude material (white solid) was dissolved in THF (20 mL) and TBAF (1.1 g, 4.2 mmol) was added. The reaction mixture was heated to reflux and was stirred at that temperature for 18 h. The reaction mixture was cooled to ambient temperature and diluted with EtOAc (100 mL). The organic phase was washed with a saturated aqueous NaHCO₃ solution (100 mL), dried (Na₂SO₄), filtered, and concentrated in vacuo. The crude material was purified by silica-gel column chromatography (petroleum ether/EtOAc, 1:1) to give ethyl 5-(1-(fluoromethyl)cyclopropyl)-1,2,4-oxadiazole-3-carboxylate as a brown oil (1.1 g, 60% yield). ¹H NMR (500 MHz, CDCl₃) δ: 4.81 (s, 1H), 4.71 (s, 1H), 4.50 (q, J=7.1 Hz, 2H), 1.71-1.66 (m, 2H), 1.46-1.42 (m, 3H), 1.42-1.38 (m, 2H).

### 4. Synthesis of 5-(1-(fluoromethyl)cyclopropyl)-1,2,4-oxadiazole-3-carboxylic acid

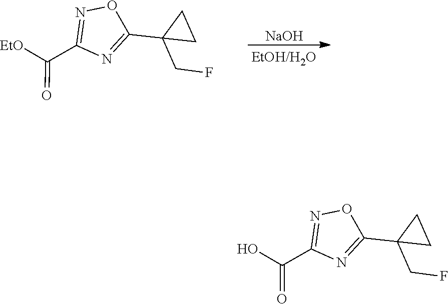 [SMILES: CCOC(=O)c1noc(C2(CF)CC2)n1 ; "NaOH" ; O=C(O)c1noc(C2(CF)CC2)n1]

To a solution of ethyl 5-[1-(fluoromethyl)cyclopropyl]-1,2,4-oxadiazole-3-carboxylate (0.1 g, 467 μmol) in MeOH (5 mL) and water (2.5 mL) was added NaOH (28 mg, 700 μmol) and the mixture was stirred at 20° C. for 2 hours. The mixture was adjusted to pH=7 with HCl (3M) and concentrated under vacuum to give 5-[1-(fluoromethyl)cyclopropyl]-1,2,4-oxadiazole-3-carboxylic acid as a white solid (80 mg, crude). ¹H NMR (400 MHz, DMSO-d₆) δ: 4.51 (s, 1H), 4.39 (s, 1H), 0.98-0.95 (m, 2H), 0.64-0.62 (m, 2H).

### 5. Synthesis of tert-butyl (E)-(4-(3-(4-(4-(dimethylamino)but-2-enoyl)piperazin-]-yl)pyridin-4-yl)-2-methylbenzyl)carbamate

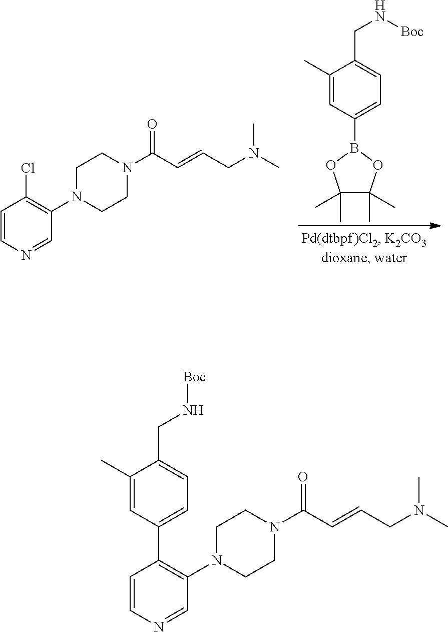

To a solution of (E)-1-(4-(4-chloropyridin-3-yl)piperazin-1-yl)-4-(dimethylamino)but-2-en-1-one (0.25 g, 810 μmol) and tert-butyl (2-methyl-4-(4,4,5,5-tetramethyl-1,3,2-dioxaborolan-2-yl)benzyl)carbamate (309 mg, 891 μmol) in dioxane (15 mL) and water (1.5 mL) was added potassium carbonate (336 mg, 2.43 mmol). Then Pd(dtbpf)Cl₂ (53 mg, 81 μmol) was added into the mixture and the mixture was stirred at 90° C. under N₂ for 4 hours. The reaction mixture was concentrated under vacuum and purified by silica gel column chromatography (DCM/MeOH=10/1) to give tert-butyl (E)-(4-(3-(4-(4-(dimethylamino)but-2-enoyl)piperazin-1-yl)pyridin-4-yl)-2-methylbenzyl)carbamate as a brown oil (270 mg, 61% yield). LCMS: m/z=494.3 (M+H⁺).

### 6. Synthesis of (E)-1-(4-(4-(4-(aminomethyl)-3-methylphenyl)pyridin-3-yl)piperazin-1-yl)-4-(dimethylamino)but-2-en-1-one trifluoroacetate

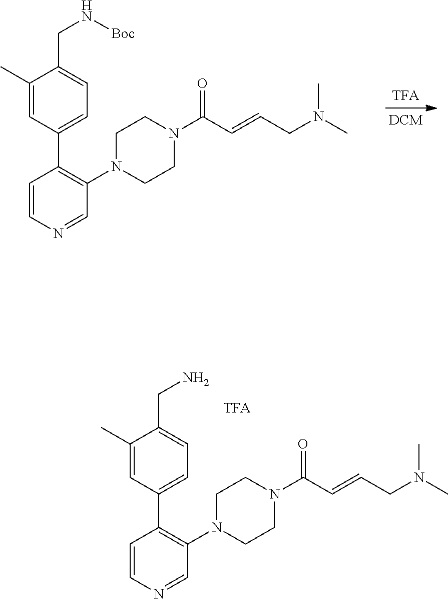

To a solution of tert-butyl (E)-(4-(3-(4-(4-(dimethylamino)but-2-enoyl)piperazin-1-yl)pyridin-4-yl)-2-methylbenzyl)carbamate (270 mg, 547 μmol) in DCM (20 mL) was added TFA (2.98 g, 26.1 mmol, 2 mL) and the mixture was stirred at 20° C. for 1 hour. The mixture was concentrated under vacuum to give crude (E)-1-(4-(4-(4-(aminomethyl)-3-methylphenyl)pyridin-3-yl)piperazin-1-yl)-4-(dimethylamino)but-2-en-1-one trifluoroacetate as an orange oil (210 mg, crude), which was carried forward without further purification. LCMS: m/z=394.3 (M+H⁺).

### 7. Synthesis of (E)-N-(4-(3-(4-(4-(dimethylamino)but-2-enoyl)piperazin-1-yl)pyridin-4-yl)-2-methylbenzyl)-5-(1-(fluoromethyl)cyclopropyl)-1,2,4-oxadiazole-3-carboxamide

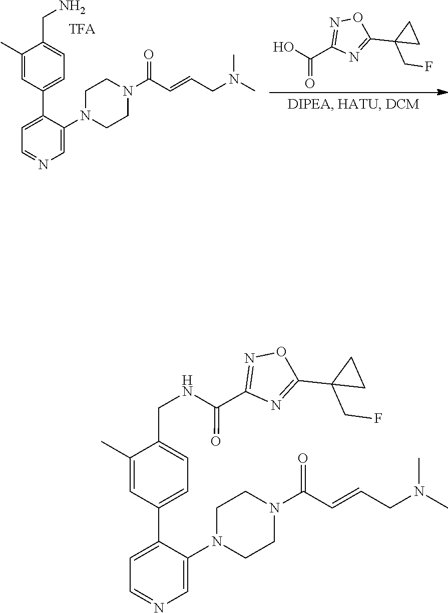 [0999] 8. Synthesis of (E)-N-(4-(3-(4-(4-(dimethylamino)but-2-enoyl)piperazin-1-yl)pyridin-4-yl)-2-methylbenzyl)-5-(1-(fluoromethyl)cyclopropyl)-1,2,4-oxadiazole-3-carboxamide was similar to that of (R,E)-5-(tert-butyl)-N-(1-(4-(3-(4-(4-(dimethylamino)but-2-enoyl)piperazin-1-yl)pyridin-4-yl)-2-methylphenyl)ethyl)-1,2,4-oxadiazole-3-carboxamide in Example 86, Step 11. The crude material was purified by prep-HPLC (Column: Welch Xtimate C18 150×25 mm×5 μm; Condition: water (10 mM NH₄HCO₃)-ACN, Begin B 27, End B 57, Gradient Time (min) 10, 100% B Hold Time (min) 2, Flow Rate (mL/min) 25.) to give (E)-N-(4-(3-(4-(4-(dimethylamino)but-2-enoyl)piperazin-1-yl)pyridin-4-yl)-2-methylbenzyl)-5-(1-(fluoromethyl)cyclopropyl)-1,2,4-oxadiazole-3-carboxamide as a white solid (34 mg 38% yield). LCMS: m/z=562.3 (M+H⁺). ¹H NMR (400 MHz, MeOH-d₄) δ=8.26-8.23 (m, 2H), 7.56-7.54 (m, 2H), 7.41 (d, J=8.4 Hz, 1H), 7.28 (d, J=5.2 Hz, 1H), 6.77-6.70 (m, 1H), 6.60-6.56 (m, 1H), 4.81-4.69 (m, 2H), 4.63 (s, 2H), 3.59-3.57 (m, 4H), 3.16 (d, J=6.4 Hz, 2H), 2.93-2.91 (m, 4H), 2.45 (s, 3H), 2.28 (s, 6H), 1.62 (t, J=4.0 Hz, 2H), 1.45 (t, J=4.4 Hz, 2H).

### Example 94: (E)-N-(4-(3-(4-(4-(dimethylamino)but-2-enoyl)piperazin-1-yl)pyridin-4-yl)-2-methylbenzyl)-3-(1-(fluoromethyl)cyclopropyl)-1,2,4-oxadiazole-5-carboxamide

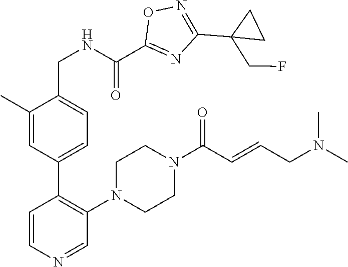

### 1. Synthesis of (E)-1-(fluoromethyl)-N′-hydroxycyclopropane-1-carboximidamide

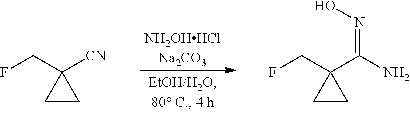 [SMILES: N#CC1(CF)CC1 ; "NH2OH·HClNa2CO3" ; N/C(=N/O)C1(CF)CC1]

To a solution of 1-(fluoromethyl)cyclopropane-1-carbonitrile (850 mg, 8.5 mmol) in EtOH (15 mL) and H₂O (1 mL) were added Na₂CO₃ (1.8 g, 17 mmol) and hydroxylamine hydrochloride (1.2 g, 17 mmol). The reaction mixture was heated to 80° C. and was stirred at that temperature for 4 h. The reaction mixture was cooled to ambient temperature, poured into H₂O (50 mL), and extracted with EtOAc (150 mL). The organic phase was dried (Na₂SO₄), filtered, and concentrated in vacuo. The crude product was purified by silica-gel column chromatography to give (E)-1-(fluoromethyl)-N′-hydroxycyclopropane-1-carboximidamide as a white solid (1 g, 90% yield), which was carried forward without further characterization or purification.

### 2. Synthesis ofethyl 3-(1-(fluoromethyl)cyclopropyl)-1,2,4-oxadiazole-5-carboxylate

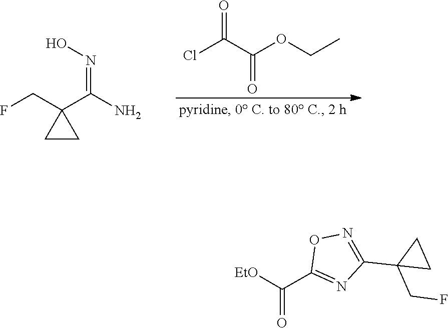

A solution of (E)-1-(fluoromethyl)-N′-hydroxycyclopropane-1-carboximidamide (5.2 g, 39 mmol) in pyridine (20 mL) was cooled to 0° C. Then ethyl chlorooxoacetate (5.3 mL, 47 mmol) was added dropwise. After the addition was complete, the reaction mixture was heated to 80° C. and was stirred at that temperature for 2 h. The reaction mixture was poured into ice-water (100 mL) and the aqueous phase was extracted with DCM (30 mL×3). The organic phase was washed with an HCl solution (30 mL, 1 M), followed by brine (30 mL). The organic phase was dried (Na₂SO₄), filtered and concentrated in vacuo to give ethyl 3-(1-(fluoromethyl)cyclopropyl)-1,2,4-oxadiazole-5-carboxylate (6.1 g, 85% yield), which was carried forward without further purification or characterization.

### 3. Synthesis of potassium 3-(1-(fluoromethyl)cyclopropyl)-1,2,4-oxadiazole-5-carboxylate

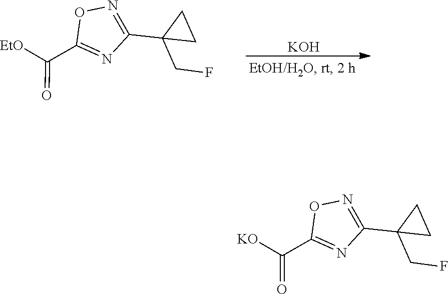

To a solution of ethyl 3-(1-(fluoromethyl)cyclopropyl)-1,2,4-oxadiazole-5-carboxylate (6.8 g, 32 mmol) in EtOH (60 mL) and water (10 mL) was added KOH (1.78 g, 32 mmol). The mixture was stirred at 20° C. for 2 h. The mixture was concentrated under reduced pressure. The crude product was triturated with ethyl acetate (50 mL) for 20 min. The solid was collected by filtration to yield potassium 3-(1-(fluoromethyl)cyclopropyl)-1,2,4-oxadiazole-5-carboxylate as a yellow solid (5.9 g, 83% yield). ESI-MS (M+H)⁺: 187.0. ¹H NMR (500 MHz, DMSO-d₆) δ: 4.72 (s, 1H), 4.62 (s, 1H), 1.20-1.26 (m, 2H), 1.13-1.19 (m, 2H).

### 4. Synthesis of 3-(1-(fluoromethyl)cyclopropyl)-N-(2-methyl-4-(4,4,5,5-tetramethyl-1,3,2-dioxaborolan-2-yl)benzyl)-1,2,4-oxadiazole-5-carboxamide

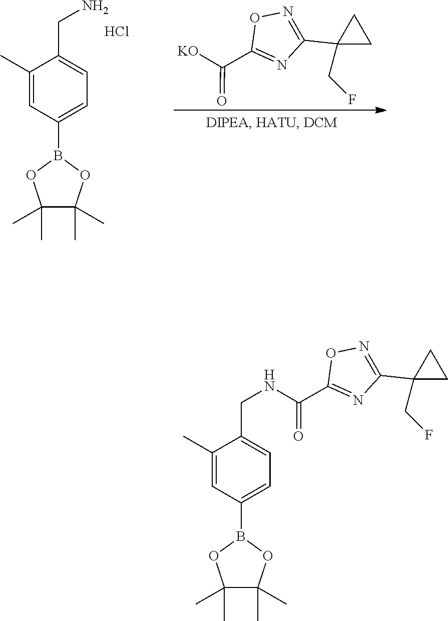

To a solution of (2-methyl-4-(4,4,5,5-tetramethyl-1,3,2-dioxaborolan-2-yl)phenyl)methanamine hydrochloride (800 mg, 2.82 mmol) in DCM (40 mL) was added DIPEA (663 mg, 5.13 mmol) at 25° C. Then potassium 3-(1-(fluoromethyl)cyclopropyl)-1,2,4-oxadiazole-5-carboxylate (477 mg, 2.56 mmol) was added into the mixture. The mixture was stirred at 25° C. for 5 min, then the HATU (1.17 g, 3.08 mmol) was added into the mixture. The mixture was stirred at 25° C. for 30 minutes. The mixture was filtered and concentrated under vacuum, then purified by prep-TLC (petroleum ether/ethyl acetate=2/1) to give 3-(1-(fluoromethyl)cyclopropyl)-N-(2-methyl-4-(4,4,5,5-tetramethyl-1,3,2-dioxaborolan-2-yl)benzyl)-1,2,4-oxadiazole-5-carboxamide as a yellow oil (700 mg, 48% yield). LCMS: m/z=416.3 (M+H⁺).

### 5. Synthesis of tert-butyl 4-(4-(4-((3-(1-(fluoromethyl)cyclopropyl)-1,2,4-oxadiazole-5-carboxamido)methyl)-3-methylphenyl)pyridin-3-yl)piperazine-1-carboxylate

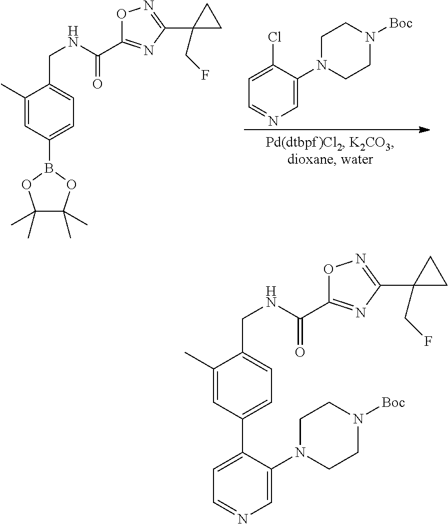 [SMILES: Cc1cc(B2OC(C)(C)C(C)(C)O2)ccc1CNC(=O)c1nc(C2(CF)CC2)no1 ; CC(C)(C)OC(=O)N1CCN(c2cnccc2Cl)CC1 ; Cc1cc(-c2ccncc2N2CCN(C(=O)OC(C)(C)C)CC2)ccc1CNC(=O)c1nc(C2(CF)CC2)no1]

To a solution of 3-(1-(fluoromethyl)cyclopropyl)-N-(2-methyl-4-(4,4,5,5-tetramethyl-1,3,2-dioxaborolan-2-yl)benzyl)-1,2,4-oxadiazole-5-carboxamide (680 mg, 1.64 mmol) in dioxane (20 mL) and water (3 mL) was added potassium carbonate (453 mg, 3.28 mmol) and tert-butyl 4-(4-chloropyridin-3-yl)piperazine-1-carboxylate (488 mg, 1.64 mmol) at 25° C. Then Pd(dtbpf)Cl₂ (120 mg, 164 μmol) was added into the mixture. The mixture was stirred at 90° C. for 2 h. The mixture was filtered and concentrated under vacuum, then was purified by column chromatography on silica gel (petroleum ether/ethyl acetate=1/0 to 1/2) to give tert-butyl 4-(4-(4-((3-(1-(fluoromethyl)cyclopropyl)-1,2,4-oxadiazole-5-carboxamido)-methyl)-3-methylphenyl)pyridin-3-yl)piperazine-1-carboxylate as a yellow oil (300 mg, 31% yield). LCMS: m/z=551.6 (M+H⁺).

### 6. Synthesis of 3-(1-(fluoromethyl)cyclopropyl)-N-(2-methyl-4-(3-(piperazin-1-yl)pyridin-4-yl)benzyl)-1,2,4-oxadiazole-5-carboxamide hydrochloride

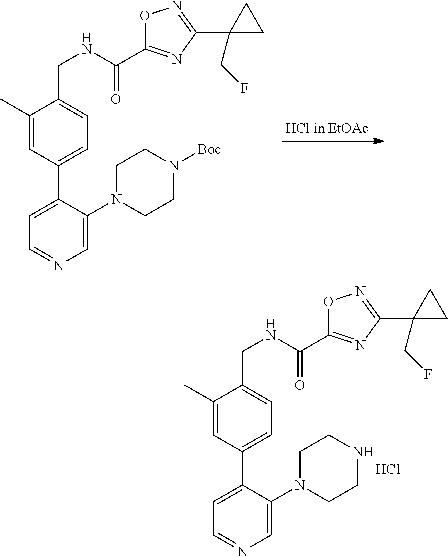 [SMILES: Cc1cc(-c2ccncc2N2CCN(C(=O)OC(C)(C)C)CC2)ccc1CNC(=O)c1nc(C2(CF)CC2)no1 ; Cc1cc(-c2ccncc2N2CCNCC2)ccc1CNC(=O)c1nc(C2(CF)CC2)no1 ; Cl]

To a solution of tert-butyl 4-(4-(4-((3-(1-(fluoromethyl)cyclopropyl)-1,2,4-oxadiazole-5-carboxamido)methyl)-3-methylphenyl)pyridin-3-yl)piperazine-1-carboxylate (300 mg, 545 μmol) in DCM (10 mL) was added an HCl solution in ethyl acetate (10 mL, 4 M) at 25° C. The mixture was stirred at 25° C. for 30 minutes. The mixture was concentrated under vacuum to give 3-(1-(fluoromethyl)cyclopropyl)-N-(2-methyl-4-(3-(piperazin-1-yl)pyridin-4-yl)benzyl)-1,2,4-oxadiazole-5-carboxamide hydrochloride as a brown solid (210 mg, 76% yield). LCMS: m/z=451.3 (M+H⁺).

### 7. Synthesis of (E)-N-(4-(3-(4-(4-(dimethylamino)but-2-enoyl)piperazin-1-yl)pyridin-4-yl)-2-methylbenzyl)-3-(1-(fluoromethyl)cyclopropyl)-1,2,4-oxadiazole-5-carboxamide

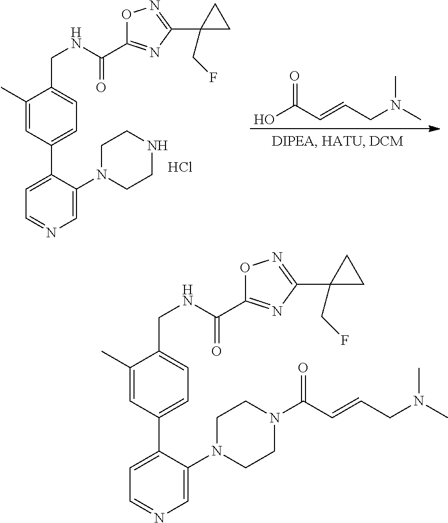

To a solution of 3-(1-(fluoromethyl)cyclopropyl)-N-(2-methyl-4-(3-(piperazin-1-yl)pyridin-4-yl)benzyl)-1,2,4-oxadiazole-5-carboxamide hydrochloride (100 mg, 205 μmol) in DCM (20 mL) was added DIPEA (53 mg, 411 μmol) at 25° C. (E)-4-(dimethylamino)but-2-enoic acid (27 mg, 205 μmol) was added to the mixture at 25° C. Then HATU (78 mg, 205 μmol) was added into the mixture. The mixture was stirred at 25° C. for 1 h. The mixture was poured into water (50 mL) and extracted with DCM (50 mL×3). The combined organic layers were washed with brine (100 mL), dried over Na₂SO₄, filtered, and concentrated under vacuum. The crude was purified by prep-HPLC (Boston Prime C18 150×30 mm×5 um, water (0.05% NH₃H₂O+10 mM NH₄HCO₃)-ACN as mobile phase, from 40-70%, Flow Rate (ml/min): 25) to give (E)-N-(4-(3-(4-(4-(dimethylamino)but-2-enoyl)piperazin-1-yl)pyridin-4-yl)-2-methylbenzyl)-3-(1-(fluoromethyl)cyclopropyl)-1,2,4-oxadiazole-5-carboxamide as a white solid (66 mg, 56% yield). LCMS: m/z=562.3 (M+H⁺). ¹H NMR DMSO-d₆) δ=9.83-9.79 (m, 1H), 8.31-8.27 (m, 2H), 7.59-7.56 (m, 2H), 7.35 (d, 1H), 7.22 (d, 1H), 6.58-6.53 (m, 2H), 4.78-4.68 (m, 2H), 4.49 (d, 2H), 3.49-3.47 (m, 4H), 2.99 (d, 2H), 2.84-2.82 (m, 4H), 2.38 (s, 3H), 2.12 (s, 6H), 1.33-1.29 (m, 4H).

### Example 95: (E)-5-(tert-butyl)-N-(4-(3-(4-(4-(dimethylamino)but-2-enoyl)piperazin-1-yl)pyridin-4-yl)-2-methylbenzyl)-4-fluoroisoxazole-3-carboxamide

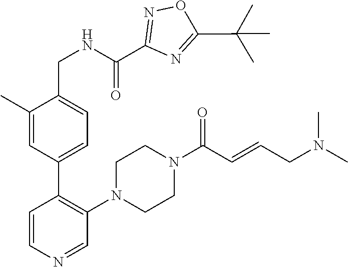

### 1. Synthesis of ethyl 5-(tert-butyl)isoxazole-3-carboxylate

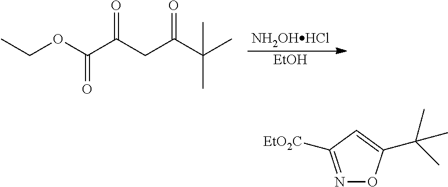

A solution of ethyl 5,5-dimethyl-2,4-dioxohexanoate (50 g, 250 mmol) and hydroxylamine hydrochloride (17.3 g, 250 mmol) in EtOH (400 mL) was stirred at 85° C. for 16 hours. After concentration, the residue was diluted with ethyl acetate (300 mL) and saturated sodium bicarbonate solution (200 mL). The organic layer was separated, and the aqueous phase was extracted with ethyl acetate (100 mL×2). The organic phases were combined and concentrated under reduced pressure to give a residue. The residue was purified by column chromatography (petroleum ether/ethyl acetate=100:1-50:1) to give 5-(tert-butyl)-4-fluoroisoxazole-3-carboxylic acid as a light-yellow oil (44 g, 89% yield). LCMS: m/z=198.1 (M+H⁺). ¹H NMR (400 MHz, DMSO-d₆) δ 6.65 (d, J=1.5 Hz, 1H), 4.35 (q, J=7.1 Hz, 2H), 1.33 (s, 9H), 1.32-1.29 (m, 3H).

### 2. Synthesis of ethyl 5-(tert-butyl)-4-fluoroisoxazole-3-carboxylate

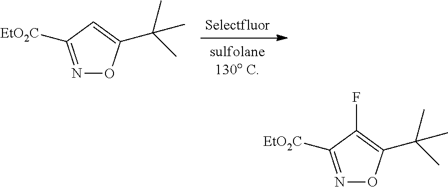

To a solution of ethyl 5-(tert-butyl)isoxazole-3-carboxylate (44 g, 233 mmol) in sulfolane (300 mL, melted in a warm water bath), Selectfluor (100 g, 283 mmol) was added and the resulting mixture was heated at 130° C. for 16 hours. The mixture was cooled to room temperature, poured into ice water (300 mL), and extracted with ethyl acetate (1.5 L). The organic layer was separated, washed with brine (500 mL×5), dried over anhydrous sodium sulfate, filtered, and concentrated under reduced pressure to give a residue. The residue was purified by silica gel column chromatography (petroleum ether/ethyl acetate=100:1) to give ethyl 5-(tert-butyl)-4-fluoroisoxazole-3-carboxylate as a light-yellow oil (8.5 g, 18% yield). ¹H NMR (400 MHz, MeOH-d₄) δ 4.43 (q, J=7.1 Hz, 2H), 1.41 (d, J=1.1 Hz, 9H), 1.39-1.34 (m, 3H).

### 3. Synthesis of 5-(tert-butyl)-4-fluoroisoxazole-3-carboxylic acid

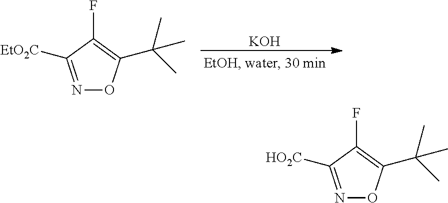

To a solution of ethyl 5-(tert-butyl)-4-fluoroisoxazole-3-carboxylate (8.5 g, 39.5 mmol) in EtOH (30 mL) and H₂O (5 mL) was added KOH (2.74 g, 41.5 mmol) and the mixture was stirred at 0° C. for 30 min. The mixture was concentrated in vacuo. The residue was purified by prep-HPLC (0.05% NH₄OH in water:acetonitrile=7.5%) to give 5-(tert-butyl)-4-fluoroisoxazole-3-carboxylic acid as a white solid (3.67 g, 50% yield). LCMS: m/z=142.1 [M−H—CO₂H]⁻. ¹H NMR (400 MHz, MeOH-d₄) δ 1.38 (d, J=1.1 Hz, 9H). ¹⁹F NMR (400 MHz, MeOH-d₄) δ 180.9 (s).

### 4. Synthesis of 5-(tert-butyl)-4-fluoro-N-(2-methyl-4-(4,4,5,5-tetramethyl-1,3,2-dioxaborolan-2-yl)benzyl)isoxazole-3-carboxamide

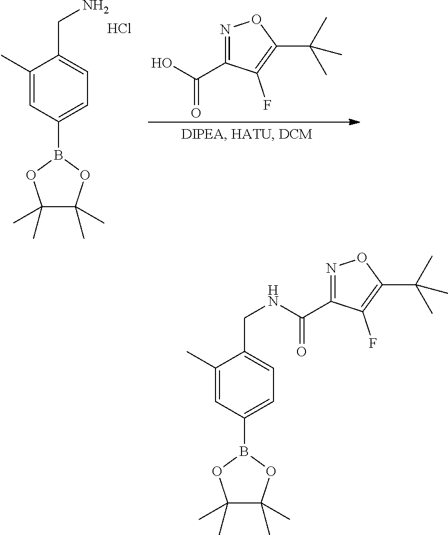

To a solution of (2-methyl-4-(4,4,5,5-tetramethyl-1,3,2-dioxaborolan-2-yl)phenyl)methanamine hydrochloride (350 mg, 1.23 mmol) in DCM (50 mL) was added DIPEA (319 mg, 2.47 mmol), 5-(tert-butyl)-4-fluoroisoxazole-3-carboxylic acid (231 mg, 1.23 mmol) and HATU (471 mg, 1.23 mmol) at 25° C. The mixture was stirred at 25° C. for 1 hour. The mixture was concentrated under vacuum and purified by silica gel chromatography (from petroleum ether to petroleum ether/ethyl acetate=3/1) to give 5-(tert-butyl)-4-fluoro-N-(2-methyl-4-(4,4,5,5-tetramethyl-1,3,2-dioxaborolan-2-yl)benzyl)isoxazole-3-carboxamide as a yellow solid (380 mg, 74% yield). LCMS: m/z=417.2 (M+H⁺).

### 5. Synthesis of tert-butyl 4-(4-(4-((5-(tert-butyl)-4-fluoroisoxazole-3-carboxamido)methyl)-3-methylphenyl)pyridin-3-yl)piperazine-1-carboxylate

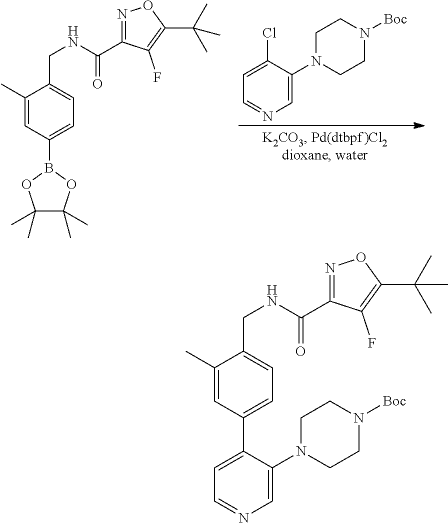 [SMILES: Cc1cc(B2OC(C)(C)C(C)(C)O2)ccc1CNC(=O)c1noc(C(C)(C)C)c1F ; CC(C)(C)OC(=O)N1CCN(c2cnccc2Cl)CC1 ; Cc1cc(-c2ccncc2N2CCN(C(=O)OC(C)(C)C)CC2)ccc1CNC(=O)c1noc(C(C)(C)C)c1F]

To a solution of 5-(tert-butyl)-4-fluoro-N-(2-methyl-4-(4,4,5,5-tetramethyl-1,3,2-dioxaborolan-2-yl)benzyl)isoxazole-3-carboxamide (350 mg, 841 μmol) in dioxane (10 mL) and water (2 mL) was added tert-butyl 4-(4-chloropyridin-3-yl)piperazine-1-carboxylate (250 mg, 841 μmol), K₂CO₃ (232 mg, 1.68 mmol) and Pd(dtbpf)Cl₂ (55 mg, 84 μmol) at 25° C. The mixture was stirred at 90° C. under N₂ for 2 hours. The mixture was concentrated under vacuum and purified by silica gel chromatography (from petroleum ether to petroleum ether/ethyl acetate=1/1) to give tert-butyl 4-(4-(4-((5-(tert-butyl)-4-fluoroisoxazole-3-carboxamido)methyl)-3-methylphenyl)pyridin-3-yl)piperazine-1-carboxylate as a yellow oil (360 mg, 78% yield). LCMS: m/z=552.3 (M+H⁺).

### 6. Synthesis of 5-(tert-butyl)-4-fluoro-N-(2-methyl-4-(3-(piperazin-1-yl)pyridin-4-yl)benzyl)isoxazole-3-carboxamide hydrochloride

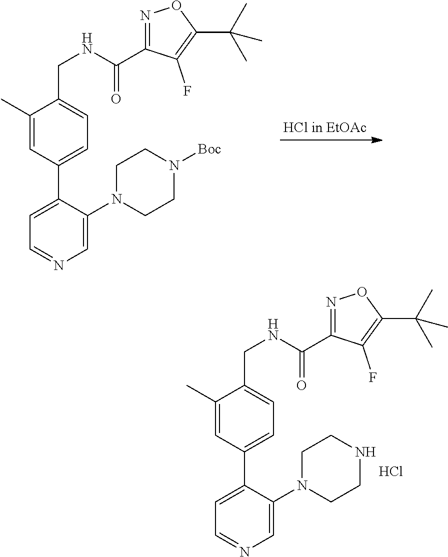 [SMILES: Cc1cc(-c2ccncc2N2CCN(C(=O)OC(C)(C)C)CC2)ccc1CNC(=O)c1noc(C(C)(C)C)c1F ; Cc1cc(-c2ccncc2N2CCNCC2)ccc1CNC(=O)c1noc(C(C)(C)C)c1F ; Cl]

To a solution of tert-butyl 4-(4-(4-((5-(tert-butyl)-4-fluoroisoxazole-3-carboxamido)methyl)-3-methylphenyl)pyridin-3-yl)piperazine-1-carboxylate (360 mg, 653 μmol) in DCM (15 mL) was added an HCl solution in ethyl acetate (8 mL, 4 M) at 25° C. The mixture was stirred at 25° C. for 1 hour. The mixture was concentrated under vacuum to give crude 5-(tert-butyl)-4-fluoro-N-(2-methyl-4-(3-(piperazin-1-yl)pyridin-4-yl)benzyl)isoxazole-3-carboxamide hydrochloride as a yellow solid (300 mg, crude), which was carried forward without further purification LCMS: m/z=452.2 (M+H⁺).

### 7. Synthesis of (E)-5-(tert-butyl)-N-(4-(3-(4-(4-(dimethylamino)but-2-enoyl)piperazin-1-yl)pyridin-4-yl)-2-methylbenzyl)-4-fluoroisoxazole-3-carboxamide

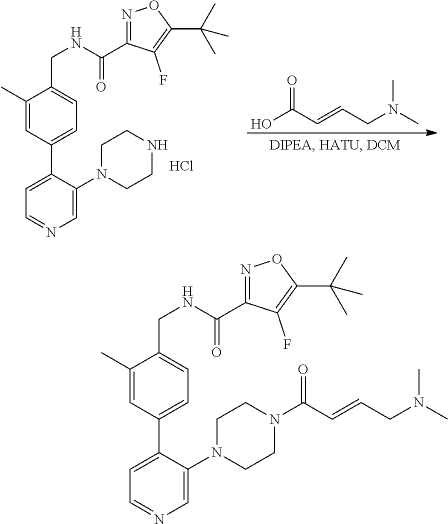

To a solution of 5-(tert-butyl)-4-fluoro-N-(2-methyl-4-(3-(piperazin-1-yl)pyridin-4-yl)benzyl)isoxazole-3-carboxamide hydrochloride (140 mg, 310 μmol) in DCM (30 mL) was added DIPEA (80 mg, 620 μmol), (E)-4-(dimethylamino)but-2-enoic acid (48 mg, 372 μmol) and HATU (118 mg, 310 μmol) at 25° C. The mixture was stirred at 25° C. for 1 h. The mixture was concentrated under vacuum and purified by prep-HPLC (Column: Welch Xtimate C18 150×25 mm×5 μm; Condition: water (10 mM NH₄HCO₃)-ACN, Begin B 40 End B 70, Gradient Time (min) 10, 100% B Hold Time (min) 2, Flow Rate (mL/min): 25) to give (E)-5-(tert-butyl)-N-(4-(3-(4-(4-(dimethylamino)but-2-enoyl)piperazin-1-yl)pyridin-4-yl)-2-methylbenzyl)-4-fluoroisoxazole-3-carboxamide as a white solid (86 mg, 49% yield). LCMS: m/z=563.3 (M+H⁺). ¹H NMR DMSO-d₆) δ=9.37 (t, 1H), 8.33-8.26 (m, 2H), 7.61-7.55 (m, 2H), 7.33 (d, 1H), 7.22 (d, 1H), 6.63-6.52 (m, 2H), 4.48 (d, 2H), 3.49-3.46 (m, 4H), 2.99 (d, 2H), 2.85-2.82 (m, 4H), 2.38 (s, 3H), 2.12 (s, 6H), 1.37 (s, 9H).

### Example 96: Synthesis of N-(4-(3-(4-acryloylpiperazin-1-yl)pyridin-4-yl)-2-methylbenzyl)-5-(tert-butyl)-4-fluoroisoxazole-3-carboxamide

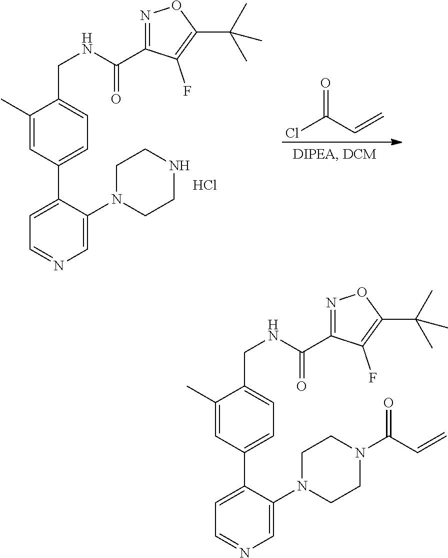

To a solution of 5-(tert-butyl)-4-fluoro-N-(2-methyl-4-(3-(piperazin-1-yl)pyridin-4-yl)benzyl)isoxazole-3-carboxamide hydrochloride (120 mg, 266 μmol) in DCM (30 mL) was added DIPEA (69 mg, 532 μmol) and acryloyl chloride (24 mg, 266 μmol) at 0° C. and the reaction mixture was stirred for 5 minutes. The mixture was quenched with MeOH (1 mL) and concentrated under vacuum. The residue was purified by prep-HPLC (Column: Welch Xtimate C18 150×25 mm×5 μm; Condition: water (10 mM NH₄HCO₃)-ACN, Begin B 40 End B 70, Gradient Time (min) 10, 100% B Hold Time (min) 2, Flow Rate (mL/min): 25) to give N-(4-(3-(4-acryloylpiperazin-1-yl)pyridin-4-yl)-2-methylbenzyl)-5-(tert-butyl)-4-fluoroisoxazole-3-carboxamide as a yellow solid (69 mg, 51% yield). LCMS: m/z=506.2 (M+H⁺). ¹H NMR (500 MHz, DMSO-d₆) δ=9.38 (t, J=6.0 Hz, 1H), 8.34-8.21 (m, 2H), 7.64-7.52 (m, 2H), 7.34 (d, J=8.5 Hz, 1H), 7.22 (d, J=5.0 Hz, 1H), 6.78-6.74 (m, 1H), 6.12-6.08 (m, 1H), 5.76-5.61 (m, 1H), 4.48 (d, J=6.0 Hz, 2H), 3.53-3.50 (m, 4H), 2.85-2.82 (m, 4H), 2.38 (s, 3H), 1.37 (s, 9H).

### Example 97: N-(4-(3-(4-acryloylpiperazin-1-yl)pyridin-4-yl)-2-methylbenzyl)-6-(difluoromethyl)nicotinamide

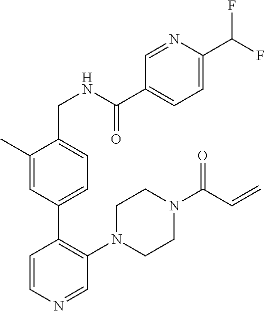 [SMILES: C=CC(=O)N1CCN(c2cnccc2-c2ccc(CNC(=O)c3ccc(C(F)F)nc3)c(C)c2)CC1]

### 1. Synthesis of 6-(difluoromethyl)-N-(2-methyl-4-(4,4,5,5-tetramethyl-1,3,2-dioxaborolan-2-yl)benzyl)nicotinamide

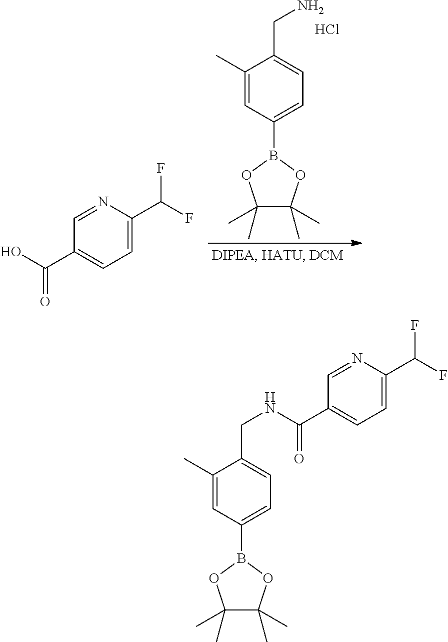

To a solution of 6-(difluoromethyl)nicotinic acid (154 mg, 0.62 mmol), (2-methyl-4-(4,4,5,5-tetramethyl-1,3,2-dioxaborolan-2-yl)phenyl)methanamine hydrochloride (90 mg, 0.52 mmol) and DIPEA (190 mg, 1.47 mmol) in DCM (9 mL) was added HATU (210 mg, 0.55 mmol) in portions at 25° C. The mixture was stirred at 25° C. for 1 hour. Water (5 mL) was added. The resulting mixture was extracted with DCM (30 mL×3). The organics were washed with water (30 mL), brine (30 mL), dried over Na₂SO₄, filtered, and concentrated to get a crude. The crude material was purified by silica gel column chromatography (eluting with ethyl acetate in petroleum ether from 0% to 50%) to give 6-(difluoromethyl)-N-(2-methyl-4-(4,4,5,5-tetramethyl-1,3,2-dioxaborolan-2-yl)benzyl)nicotinamide as a pale yellow solid (220 mg, 95% yield). LCMS: m/z=403.2 (M+H⁺).

### 2. Synthesis of tert-butyl 4-(4-(4-((6-(difluoromethyl)nicotinamido)methyl)-3-methylphenyl)pyridin-3-yl)piperazine-1-carboxylate

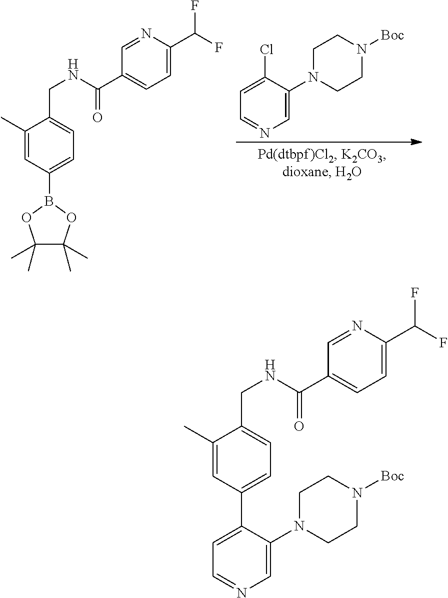 [SMILES: Cc1cc(B2OC(C)(C)C(C)(C)O2)ccc1CNC(=O)c1ccc(C(F)F)nc1 ; CC(C)(C)OC(=O)N1CCN(c2cnccc2Cl)CC1 ; Cc1cc(-c2ccncc2N2CCN(C(=O)OC(C)(C)C)CC2)ccc1CNC(=O)c1ccc(C(F)F)nc1]

The mixture of 6-(difluoromethyl)-N-(2-methyl-4-(4,4,5,5-tetramethyl-1,3,2-dioxaborolan-2-yl)benzyl)nicotinamide (163 mg, 0.55 mmol), tert-butyl 4-(4-chloropyridin-3-yl)piperazine-1-carboxylate (200 mg, 0.50 mmol), K₂CO₃ (206 mg, 1.5 mmol) and Pd(dtbpf)Cl₂ (32 mg, 0.05 mmol) in dioxane (15 mL) and water (3 mL) was bubbled with N₂ for 1 min. The mixture was stirred at 90° C. for 4 hours. The reaction mixture was concentrated and the residue was purified via silica gel column chromatography (eluting with ethyl acetate in petroleum ether from 0% to 100%) to give tert-butyl 4-(4-(4-((6-(difluoromethyl)nicotinamido)methyl)-3-methylphenyl)pyridin-3-yl)piperazine-1-carboxylate as a colorless oil (290 mg, 98% yield). ¹H NMR: (500 MHz, METHANOL-d₄) δ=9.11 (d, J=1.5 Hz, 1H), 8.43 (dd, J₁=2.0 Hz, J₂=8.0 Hz, 1H), 8.26 (d, J=3.0 Hz, 2H), 7.84 (d, J=8.0 Hz, 1H), 7.61-7.56 (m, 2H), 7.46 (d, J=8.0 Hz, 1H), 7.29 (d, J=5.0 Hz, 1H), 6.92-6.69 (m, 1H), 4.70 (s, 2H), 3.38-3.34 (m, 4H), 2.91-2.86 (m, 4H), 2.49 (s, 3H), 1.45 (s, 9H).

### 3. Synthesis of 6-(difluoromethyl)-N-(2-methyl-4-(3-(piperazin-1-yl)pyridin-4-yl)benzyl)nicotinamide hydrochloride

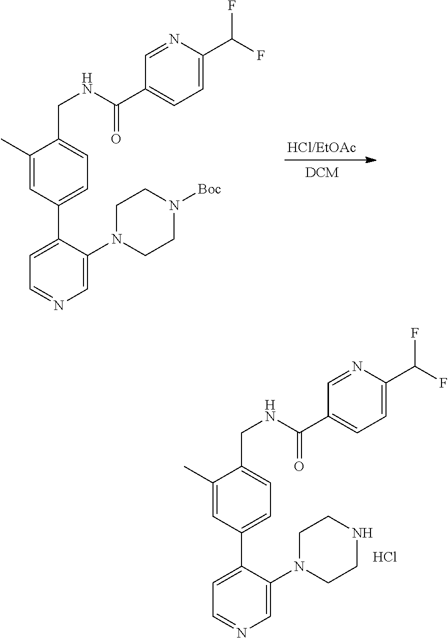 [SMILES: Cc1cc(-c2ccncc2N2CCN(C(=O)OC(C)(C)C)CC2)ccc1CNC(=O)c1ccc(C(F)F)nc1 ; Cc1cc(-c2ccncc2N2CCNCC2)ccc1CNC(=O)c1ccc(C(F)F)nc1 ; Cl]

To a mixture of tert-butyl 4-(4-(4-((6-(difluoromethyl)nicotinamido)methyl)-3-methylphenyl)pyridin-3-yl)piperazine-1-carboxylate (270 mg, 0.49 mmol) in DCM (1 mL) was added an HCl solution in ethyl acetate (4 M, 12 mL). The mixture was stirred at 25° C. for 1 hour. The reaction mixture was concentrated to give crude 6-(difluoromethyl)-N-(2-methyl-4-(3-(piperazin-1-yl)pyridin-4-yl)benzyl)nicotinamide hydrochloride as a yellow oil (220 mg, crude), which was carried forward without further purification. LCMS: m/z=438.2 (M+H⁺).

### 4. Synthesis of N-(4-(3-(4-acryloylpiperazin-1-yl)pyridin-4-yl)-2-methylbenzyl)-6-(difluoromethyl)nicotinamide

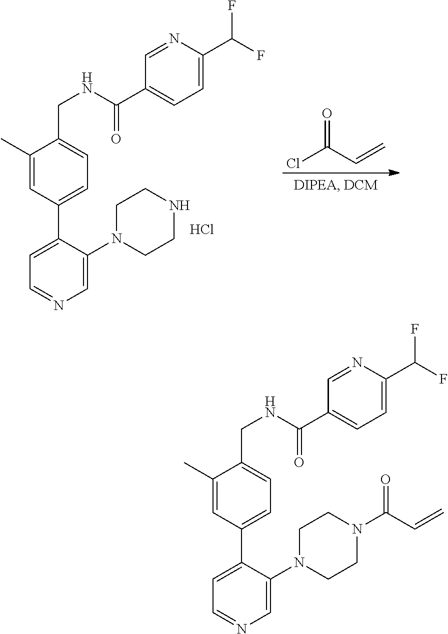

To the mixture of 6-(difluoromethyl)-N-(2-methyl-4-(3-(piperazin-1-yl)pyridin-4-yl)benzyl)nicotinamide hydrochloride (110 mg, 0.25 mmol) and DIPEA (97 mg, 0.75 mmol) in DCM (10 mL) was added acryloyl chloride (23 mg, 0.25 mmol) at 0° C. The mixture was stirred at 0° C. for 2 min. MeOH (1 mL) was added dropwise. The resulting mixture was stirred at 25° C. for 10 min. The solvent was removed and the material was purified by prep HPLC (Column: Boston Prime C18 150×30 mm×5 μm; Condition: water (0.05% NH₃H₂O+10 mM NH₄HCO₃)—ACN, Begin B 28, End B 58, Gradient Time (min) 10, 100% B Hold Time (min) 2, Flow Rate (mL/min) 25) to give N-(4-(3-(4-acryloylpiperazin-1-yl)pyridin-4-yl)-2-methylbenzyl)-6-(difluoromethyl)nicotinamide as a white solid (63 mg, 51% yield). LCMS: m/z=492.0 (M+H⁺). ¹H NMR (500 MHz, METHANOL-d₄) δ=9.11 (d, J=1.5 Hz, 1H), 8.43 (dd, J₁=2.0 Hz, J₂=8.0 Hz, 1H), 8.32-8.22 (m, 2H), 7.84 (d, J=8.0 Hz, 1H), 7.63-7.57 (m, 2H), 7.47 (d, J=8.5 Hz, 1H), 7.30 (d, J=5.0 Hz, 1H), 6.94-6.67 (m, 2H), 6.20 (dd, J₁=2.0 Hz, J₂=17.0 Hz, 1H), 5.75 (dd, J₁=2.0 Hz, J₂=10.5 Hz, 1H), 4.69 (s, 2H), 3.61-3.59 (m, 4H), 2.95-2.93 (m, 4H), 2.49 (s, 3H).

### Example 98: N-(4-(3-(4-acryloylpiperazin-1-yl)pyridin-4-yl)-2-methylbenzyl)-5-(1-(fluoromethyl)cyclopropyl)-1,2,4-oxadiazole-3-carboxamide

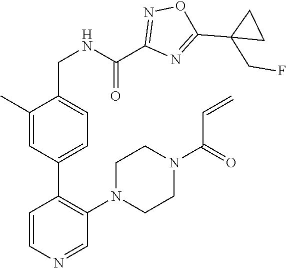 [SMILES: C=CC(=O)N1CCN(c2cnccc2-c2ccc(CNC(=O)c3noc(C4(CF)CC4)n3)c(C)c2)CC1]

### 1. Synthesis of 1-(4-(4-chloropyridin-3-yl)piperazin-1-yl)prop-2-en-1-one

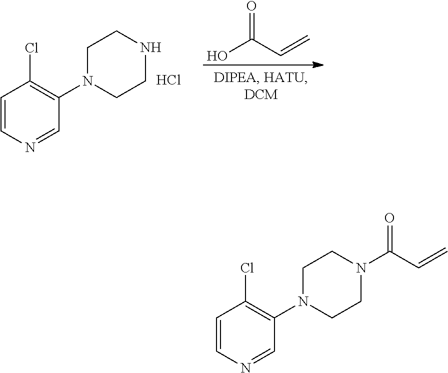

To a solution of 1-(4-chloro-3-pyridyl)piperazine hydrochloride (600 mg, 2.56 mmol) in DCM (50 mL) was added DIPEA (662 mg, 5.13 mmol, 893 μL) at 15° C. Then the prop-2-enoyl chloride (232 mg, 2.56 mmol, 208 μL) was added into the mixture at 0° C. The mixture was stirred at 0° C. for 30 minutes. The mixture was quenched with MeOH (5 mL). The mixture was concentrated under the vacuum to give the crude product, which was purified by prep-HPLC (Welch Xtimate C18 150×25 mm×5 μm, water (10 mM NH₄HCO₃)-ACN as mobile phase, from 30-60%, Flow Rate (ml/min): 25) to give 1-[4-(4-chloro-3-pyridyl)piperazin-1-yl]prop-2-en-1-one as a white solid (320 mg, 49% yield). LCMS: m/z=252.0 (M+H⁺).

### 2. Synthesis of tert-butyl (4-(3-(4-acryloylpiperazin-1-yl)pyridin-4-yl)-2-methylbenzyl)carbamate

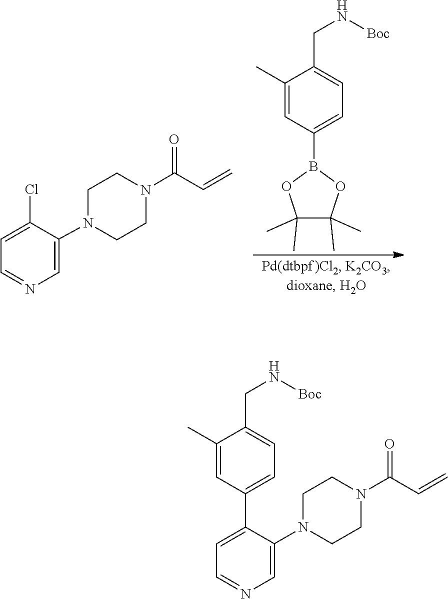 [SMILES: C=CC(=O)N1CCN(c2cnccc2Cl)CC1 ; Cc1cc(B2OC(C)(C)C(C)(C)O2)ccc1CNC(=O)OC(C)(C)C ; C=CC(=O)N1CCN(c2cnccc2-c2ccc(CNC(=O)OC(C)(C)C)c(C)c2)CC1]

To a solution of 1-[4-(4-chloro-3-pyridyl)piperazin-1-yl]prop-2-en-1-one (300 mg, 1.19 mmol) in dioxane (20 mL) and water (3 mL) was added tert-butyl N-[[2-methyl-4-(4,4,5,5-tetramethyl-1,3,2-dioxaborolan-2-yl)phenyl]methyl]carbamate (828 mg, 2.38 mmol) and potassium carbonate (329 mg, 2.38 mmol) at 15° C. Then Pd(dtbpf)Cl₂ (155 mg, 238 μmol) was added to the mixture at 15° C. The mixture was stirred at 90° C. for 2 hours. The mixture was filtered and concentrated under vacuum to give the crude, which was purified by prep-HPLC (Column: Welch Xtimate C18 150×25 mm×5 μm; Condition: water (10 mM NH₄HCO₃)-ACN, Begin B 36, End B 66, Gradient Time (min) 10, 100% B Hold Time (min) 2, Flow Rate (mL/min) 25.) to give tert-butyl (4-(3-(4-acryloylpiperazin-1-yl)pyridin-4-yl)-2-methylbenzyl)carbamate as a white solid (380 mg, 73% yield). LCMS: m/z=437.5 (M+H⁺).

### 3. Synthesis of 1-(4-(4-(4-(aminomethyl)-3-methylphenyl)pyridin-3-yl)piperazin-1-yl)prop-2-en-1-one 2,2,2-trifluoroacetate

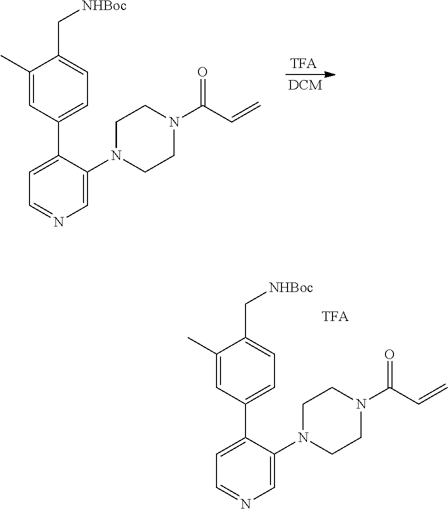 [SMILES: C=CC(=O)N1CCN(c2cnccc2-c2ccc(CNC(=O)OC(C)(C)C)c(C)c2)CC1 ; C=CC(=O)N1CCN(c2cnccc2-c2ccc(CNC(=O)OC(C)(C)C)c(C)c2)CC1]

A solution of tert-butyl N-[[2-methyl-4-[3-(4-prop-2-enoylpiperazin-1-yl)-4-pyridyl]phenyl]methyl]-carbamate (360 mg, 825 μmol) in TFA (1 mL) and DCM (10 mL) was stirred at 15° C. for 30 minutes. The mixture was concentrated under the vacuum to give crude 1-(4-(4-(4-(aminomethyl)-3-methylphenyl)-pyridin-3-yl)piperazin-1-yl)prop-2-en-1-one 2,2,2-trifluoroacetate as a yellow oil (200 mg, crude), which was carried forward without further purification. LCMS: m/z=337.2 (M+H⁺).

### 4. Synthesis of N-(4-(3-(4-acryloylpiperazin-1-yl)pyridin-4-yl)-2-methylbenzyl)-6-(difluoromethyl)nicotinamide

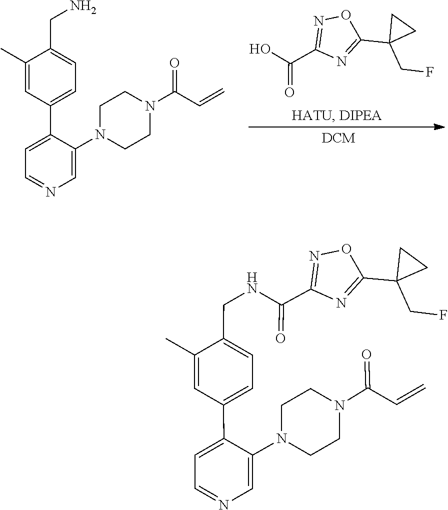 [SMILES: C=CC(=O)N1CCN(c2cnccc2-c2ccc(CN)c(C)c2)CC1 ; O=C(O)c1noc(C2(CF)CC2)n1 ; C=CC(=O)N1CCN(c2cnccc2-c2ccc(CNC(=O)c3noc(C4(CF)CC4)n3)c(C)c2)CC1]

To a solution of 1-[4-[4-[4-(aminomethyl)-3-methyl-phenyl]-3-pyridyl]piperazin-1-yl]prop-2-en-1-one trifluoroacetate (0.1 g, 297 μmol) in DCM (50 mL) was added DIPEA (115 mg, 892 μmol, 155 μL). Then 5-[1-(fluoromethyl)cyclopropyl]-1,2,4-oxadiazole-3-carboxylic acid (66 mg, 357 μmol) and HATU (113 mg, 297 μmol) was added into the mixture and the mixture was stirred at 20° C. for 1 hour. The reaction mixture was concentrated under vacuum to give a crude, which was purified by prep-HPLC (Column: Welch Xtimate C18 150×25 mm×5 μm; Condition: water (10 mM NH₄HCO₃)-ACN, Begin B 29, End B 59, Gradient Time (min) 10, 100% B Hold Time (min) 2, Flow Rate (mL/min) 25.) to give N-(4-(3-(4-acryloylpiperazin-1-yl)pyridin-4-yl)-2-methylbenzyl)-5-(1-(fluoromethyl)cyclopropyl)-1,2,4-oxadiazole-3-carboxamide as a white solid (53 mg, 35% yield) LCMS: m/z=505.2 (M+H⁺). ¹H NMR (400 MHz, DMSO-d₆) δ=9.44 (t, J=6.4 Hz, 1H), 8.30-8.27 (m, 2H), 7.59-7.57 (m, 2H), 7.30 (d, J=8. Hz, 1H), 7.22 (d, J=4.8 Hz, 1H), 6.80-6.73 (m, 1H), 6.10 (d, J=16.4 Hz, 1H), 5.66 (d, J=10.4 Hz, 1H), 4.83-4.71 (m, 2H), 4.48 (d, J=6.0 Hz, 2H), 3.50-3.34 (m, 4H), 2.84-2.81 (m, 4H), 2.37 (s, 3H), 1.55-1.46 (m, 4H).

### Example 99: N-(4-(3-(4-acryloylpiperazin-1-yl)pyridin-4-yl)-2-methylbenzyl)-5-(1-(difluoromethyl)cyclopropyl)-1,2,4-oxadiazole-3-carboxamide

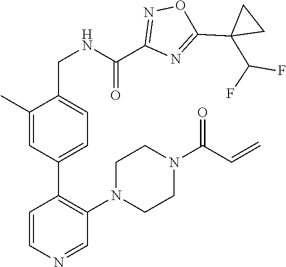 [SMILES: C=CC(=O)N1CCN(c2cnccc2-c2ccc(CNC(=O)c3noc(C4(C(F)F)CC4)n3)c(C)c2)CC1]

### 1. Synthesis of ethyl 2-amino-2-(hydroxyimino)acetate

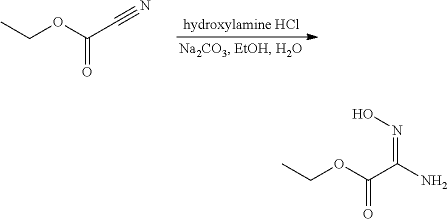

To a solution of ethyl carbonocyanidate (50 g, 505 mmol), hydroxylamine hydrochloride (52.6 g, 757 mmol) and Na₂CO₃ (41.2 g, 389 mmol) in EtOH (500 mL) was added dropwise H₂O (300 mL) at 20° C. The mixture was stirred at 20° C. for 2 h. The mixture was concentrated and then water (300 mL) was added. The mixture was extracted with DCM (3×300 mL). The combined organic layers were washed with brine (100 mL), dried over Na₂SO₄, filtered and concentrated. The crude product was triturated with DCM (100 mL) and petroleum ether (500 mL) for 20 min. The solid was collected by filtration to give ethyl 2-amino-2-(hydroxyimino)acetate as a yellow oil (30 g, 45% yield). ¹H NMR (400 MHz, CDCl₃) δ ppm 8.92-9.31 (m, 1H), 5.12 (s, 2H), 4.33 (q, J=7.2 Hz, 2H), 1.36 (t, J=7.2 Hz, 3H).

### 2. Synthesis of 1-(hydroxymethyl)cyclopropane-1-carboxylic acid

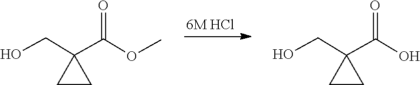

A solution of 1-(methoxymethyl)cyclopropane-1-carboxylic acid (28 g, 215 mmol) in an aqueous HCl solution (6 M, 124 mL) was stirred at 60° C. for 2 h. The residue was concentrated under reduced pressure to yield 1-(hydroxymethyl)cyclopropane-1-carboxylic acid as a yellow oil (24 g, 96% yield), which was used in next step without further purification. ¹H NMR (400 MHz, DMSO-d₆) δ ppm 3.53 (s, 2H), 0.92-0.98 (m, 2H), 0.76-0.84 (m, 2H).

### 3. Synthesis of ethyl 2-(hydroxyimino)-2-(1-(hydroxymethyl)cyclopropane-1-carboxamido)acetate

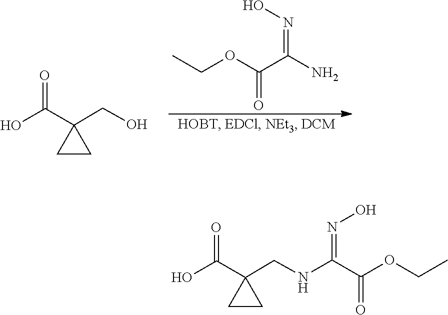

To a solution of 1-(hydroxymethyl)cyclopropane-1-carboxylic acid (24 g, 207 mmol) in DCM (500 mL) was added ethyl 2-amino-2-(hydroxyimino)acetate (27.3 g, 207 mmol), triethylamine (62.8 g, 620 mmol, 86 mL), EDCI (47.6 g, 248 mmol) and HOBT (33.5 g, 248 mmol). The mixture was stirred at 20° C. for 12 h under N₂ atmosphere. The mixture was concentrated under reduced pressure and the residue was purified on silica gel column chromatography (petroleum ether/ethyl acetate=10/1 to 1/1) to yield ethyl 2-(hydroxyimino)-2-(1-(hydroxymethyl)cyclopropane-1-carboxamido)acetate as a yellow oil (15 g, 32% yield). ¹H NMR (400 MHz, CDCl₃) δ ppm 5.70 (s, 2H), 4.36 (q, J=7.2 Hz, 2H), 3.71 (s, 2H), 2.75 (s, 1H), 1.33-1.39 (m, 5H), 0.92-0.97 (m, 2H).

### 4. Synthesis of ethyl 5-(1-(hydroxymethyl)cyclopropyl)-1,2,4-oxadiazole-3-carboxylate

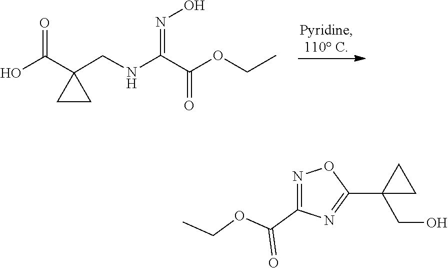

A solution of ethyl 2-(hydroxyimino)-2-(1-(hydroxymethyl)cyclopropane-1-carboxamido)acetate (15 g, 65.2 mmol) in pyridine (193 g, 2.44 mol, 197 mL) was stirred at 110° C. for 12 h under N₂ atmosphere. The mixture was concentrated under reduced pressure and the residue was purified on silica gel column chromatography (petroleum ether/ethyl acetate=10/1 to 1/1) to yield ethyl 5-(1-(hydroxymethyl)-cyclopropyl)-1,2,4-oxadiazole-3-carboxylate as a yellow oil (5.7 g, 41% yield). ¹H NMR: (400 MHz, MeOH-d₄) δ ppm 4.46 (q, J=7.2 Hz, 2H), 3.94 (s, 2H), 1.45-1.50 (m, 2H), 1.42 (t, J=7.2 Hz, 3H), 1.31-1.34 (m, 2H).

### 5. Synthesis of ethyl 5-(1-formylcyclopropyl)-1,2,4-oxadiazole-3-carboxylate

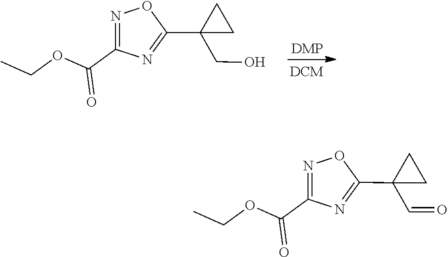

To a solution of ethyl 5-(1-(hydroxymethyl)cyclopropyl)-1,2,4-oxadiazole-3-carboxylate (5.6 g, 26.4 mmol) in DCM (60 mL) was added Dess Martin periodinane (14.6 g, 34.3 mmol) at 0° C. under N₂ atmosphere. The reaction mixture was stirred 20° C. of 12 h. Water (100 mL) was added, the mixture was filtered, and the filtrate was extracted with DCM (3×50 mL). The combined organic layers were washed with saturated aqueous NaHCO₃ solution (3×200 mL), saturated aqueous Na₂SO₃ solution (3×200 mL) and brine (300 mL), dried over Na₂SO₄, filtered, and concentrated to yield ethyl 5-(1-formylcyclopropyl)-1,2,4-oxadiazole-3-carboxylate as a yellow oil (5.1 g, 92% yield), which was used in next step without further purification. ¹H NMR (400 MHz, CDCl₃) δ ppm 10.28 (br s, 1H), 4.51 (q, J=7.2 Hz, 2H), 1.98-2.03 (m, 2H), 1.92-1.98 (m, 2H), 1.44 (t, J=7.2 Hz, 3H).

### 6. Synthesis ofethyl 5-(1-(difluoromethyl)cyclopropyl)-1,2,4-oxadiazole-3-carboxylate

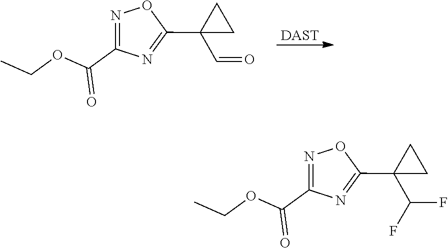

To a solution of ethyl 5-(1-formylcyclopropyl)-1,2,4-oxadiazole-3-carboxylate (4.8 g, 22.8 mmol) in DCM (60 mL) was added DAST (7.36 g, 45.7 mmol, 6.03 mL) in DCM (10 mL) dropwise at 0° C. under N₂ atmosphere. The mixture was stirred at 20° C. for 2 h. To the mixture was added saturated NaHCO₃ solution (50 mL), and the layers were separated. The aqueous phase was extracted with DCM (3×50 mL). The combined organic layers were washed with brine (200 mL), dried over Na₂SO₄, filtered and concentrated under reduced pressure to yield ethyl 5-(1-(difluoromethyl)cyclopropyl)-1,2,4-oxadiazole-3-carboxylate as a yellow oil (4.2 g, 79% yield), which was used in next step without further purification. ¹H NMR: (400 MHz, CDCl₃) δ ppm 6.53 (t, J=56.8 Hz, 1H), 4.49 (q, J=7.2 Hz, 2H), 1.71-1.88 (m, 4H), 1.42 (t, J=7.2 Hz, 3H).

### 7. Synthesis of potassium 5-(1-(difluoromethyl)cyclopropyl)-1,2,4-oxadiazole-3-carboxylate

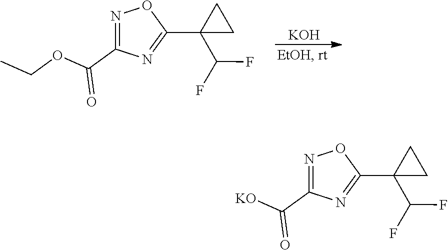

To a solution of ethyl 5-(1-(difluoromethyl)cyclopropyl)-1,2,4-oxadiazole-3-carboxylate (4.2 g, 18 mmol) in EtOH (30 mL) and H₂O (10 mL) was added KOH (1.01 g, 18 mmol). The mixture was stirred at 20° C. for 2 h. The mixture was concentrated under reduced pressure. The crude product was triturated with EtOAc (50 mL) for 20 min. The solid was collected and purified by prep-HPLC (column: YMC-Actus Triart C18 150*30 mm*7 μm; Mobile phase: Water-ACN; B %: 0-20, 9 min) Flow Rate (ml/min) 25 followed by lyophilization to yield potassium 5-(1-(difluoromethyl)cyclopropyl)-1,2,4-oxadiazole-3-carboxylate as a white solid (1.51 g, 35% yield). LCMS (M-COOK-H⁺=159.0). ¹H NMR: (400 MHz, DMSO-d₆) δ ppm 6.56 (t, J=58.4 Hz, 1H), 0.93-0.97 (m, 2H), 0.76-0.79 (m, 2H).

### 8. Synthesis of N-(4-(3-(4-acryloylpiperazin-1-yl)pyridin-4-yl)-2-methylbenzyl)-5-(1-(difluoromethyl)cyclopropyl)-1,2,4-oxadiazole-3-carboxamide

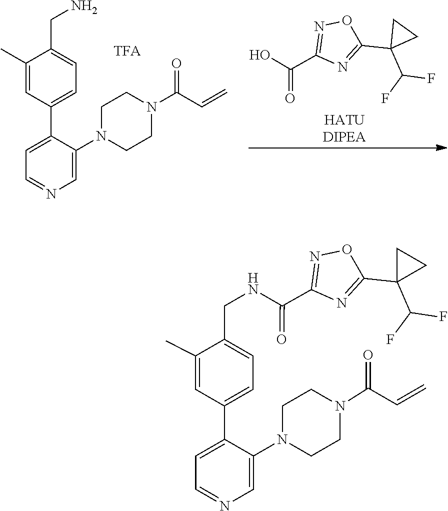 [SMILES: C=CC(=O)N1CCN(c2cnccc2-c2ccc(CN)c(C)c2)CC1 ; O=C(O)c1noc(C2(C(F)F)CC2)n1 ; C=CC(=O)N1CCN(c2cnccc2-c2ccc(CNC(=O)c3noc(C4(C(F)F)CC4)n3)c(C)c2)CC1]

To a solution of 1-[4-[4-[4-(aminomethyl)-3-methyl-phenyl]-3-pyridyl]piperazin-1-yl]prop-2-en-1-one trifluoroacetate (0.1 g, 297 μmol) in DCM (30 mL) was added DIPEA (58 mg, 445 μmol). Then 5-[1-(fluoromethyl)cyclopropyl]-1,2,4-oxadiazole-3-carboxylic acid (45 mg, 223 μmol) and HATU (113 mg, 297 μmol) were added into the mixture and the mixture was stirred at 20° C. for 1 hour. The reaction mixture was concentrated under vacuum to give a crude, which was purified by prep-HPLC (Column: Welch Xtimate C18 150×25 mm×5 μm; Condition: water (10 mM NH₄HCO₃)-ACN, Begin B 31, End B 61, Gradient Time (min) 10, 100% B Hold Time (min) 2, Flow Rate (mL/min) 25.) to give N-(4-(3-(4-acryloylpiperazin-1-yl)pyridin-4-yl)-2-methylbenzyl)-5-(1-(difluoromethyl)cyclopropyl)-1,2,4-oxadiazole-3-carboxamide as a white solid (72 mg, 62% yield). LCMS: m/z=523.3 (M+H⁺). ¹H NMR (500 MHz, MeOH-d₄) δ=8.26-8.24 (m, 2H), 7.58-7.56 (m, 2H), 7.42 (d, J=8.0 Hz, 1H), 7.29 (d, J=4.5 Hz, 1H), 6.75-6.68 (m, 1H), 6.44 (s, 1H), 6.19-6.17 (m, 1H), 5.75-5.72 (m, 1H), 4.64 (s, 2H), 3.59-3.57 (m, 4H), 2.94-2.91 (m, 4H), 2.45 (s, 3H), 1.63 (s, 4H).

### In Vitro BTK Kinase Assay: Btk-PolyGAT-LS Assay

The purpose of the BTK in vitro assay is to determine compound potency against BTK through the measurement of IC₅₀. Compound inhibition is measured after monitoring the amount of phosphorylation of a fluorescein-labeled polyGAT peptide (Invitrogen PV3611) in the presence of active BTK enzyme (Upstate 14-552), ATP, and inhibitor. The BTK kinase reaction was done in a black 96 well plate (costar 3694). For a typical assay, a 24 μL aliquot of a ATP/peptide master mix (final concentration; ATP 10 μM, polyGAT 100 nM) in kinase buffer (10 mM Tris-HCl pH 7.5, 10 mM MgCl2, 200 μM Na₃PO₄, 5 mM DTT, 0.01% Triton X-100, and 0.2 mg/ml casein) is added to each well. Next, I pL of a 4-fold, 40× compound titration in 100% DMSO solvent is added, followed by adding 15 μL of BTK enzyme mix in 1× kinase buffer (with a final concentration of 0.25 nM). The assay is incubated for 30 minutes before being stopped with 28 μL of a 50 mM EDTA solution. Aliquots (5 μL) of the kinase reaction are transferred to a low volume white 384 well plate (Coming 3674), and 5 μL of a 2× detection buffer (Invitrogen PV3574, with 4 nM Tb-PY20 antibody, Invitrogen PV3552) is added. The plate is covered and incubated for 45 minutes at room temperature. Time resolved fluorescence (TRF) on Molecular Devices M5 (332 nm excitation; 488 nm emission; 518 nm fluorescein emission) is measured. IC₅₀ values are calculated using a four parameter fit with 100% enzyme activity determined from the DMSO control and 0% activity from the EDTA control.

Table 2 shows the activity of selected compounds of this invention in the in vitro Btk kinase assay, wherein each compound number corresponds to the compound numbering set forth in Examples 1-99 described herein.
“†” represents an IC₅₀ of greater than 10 nM (10 nM<IC₅₀).
“††” represents an IC₅₀ of greater than 1 nM and equal to or less than 10 nM (1 nM<IC₅₀<10 nM).
“†††” represents an IC₅₀ of equal to or less than 1 nM (IC₅₀ 1 nM)

TABLE 2

| IC₅₀ (nM) | Example No. |
|---|---|
| † | 4, 5, 6, 7, 10, 11, 15, 25, 27, 28, 31, 34, 53 Peak 2, 54, 55 |
| †† | 2, 3, 21, 23, 24, 26, 29, 30, 32, 33, 35, 36, 40, |
| | 41, 43, 44, 52, 53 Peak 1, 59, 64, |
| | 65, 66, 67, 68, 69, 70, 71, 77, 81, 86, 88, 95, 97 |
| ††† | 1, 8, 9, 12, 13, 14, 16, 17, 18, 19, 20, 22, |
| | 37, 38, 39, 42, 45, 46, 47, 48, 49, 50, 51, |
| | 56, 57, 58, 60, 61, 62, 63, 72, 73, 74, 75, |
| | 76, 78, 79, 80, 82, 83, 84, 85, 87, 89, 90, |
| | 91, 92, 93, 94, 96, 98, 99 |

In Vitro whole blood CD69 Assay

Human heparinized venous blood from health donors was aliquoted into 96-well plate and “spiked” with serial dilutions of formula I compounds in DMSO or with DMSO without drug. The final concentration of DMSO in all wells was 0.1%. The plate was incubated at 37° C. for 30 min. Drug-containing samples were stimulated with 0.1 pg/mL mouse anti-human IgD-dextran (1A62) or 20 pg/mL polyclonal rabbit F(ab′)₂ anti-human IgD. Phosphate-buffered saline (PBS) was added to the negative control unstimulated sample and the plates were incubated overnight (18 to 22 hours) at 37° C. Cells were stained with fluorochrome-conjugated anti-CD19 and anti-CD69 antibodies. Lyse/fix solution was used to remove red blood cells by hypotonic lysis and to fix the remaining cells, which were then analyzed by flow cytometry. CD19+ B cells were gated and analyzed for CD69 expression. The percentage of B cells expressing CD69 was plotted versus the log 10 of the concentration of the drug and the best-fit curves (variable Hill slope) were generated to obtain the IC50 value.

Table 3 shows the activity of selected compounds of this invention in the Whole Blood CD69 inhibition assay, wherein each compound number corresponds to the compound numbering set forth in Examples 1-99 described herein.
“*” represents an IC₅₀ of greater than 1 μM (1 μM<IC₅₀).
“**” represents an IC₅₀ of greater than 0.1 μM and equal to or less than 1 μM (0.1 μM<IC₅₀ 1 μM).
“***” represents an IC₅₀ of equal to or less than 0.1 μM (IC₅₀ 0.1 μM)

TABLE 3

| IC₅₀ (μM) | Compound No. |
|---|---|
| * | 3, 15, 17, 23, 24, 28, 29, 30, 33, 40, 41, 43, 44, 51, 53 Peak 1 |
| ** | 1, 2, 14, 16, 18, 19, 26, 32, 35, 36, 37, 39, 42, |
| | 46, 49, 52, 56, 63, 69, 71, 73, 86, 94, 95 |
| *** | 8, 12, 13, 22, 61, 82, 85, 90 |

### Other Embodiments

All of the features disclosed in this specification may be combined in any combination. Each feature disclosed in this specification may be replaced by an alternative feature serving the same, equivalent or similar purpose. Thus, unless expressly stated otherwise, each feature disclosed is only an example of a series of equivalent or similar features.

From the above description, one skilled in the art can easily ascertain the essential characteristics of the present invention, and without departing from the spirit and scope thereof, can make various changes and modifications of the invention to adapt it to various usage and conditions. Thus, other embodiments are also within the scope of the following claims.

1. A compound represented by Formula (I): 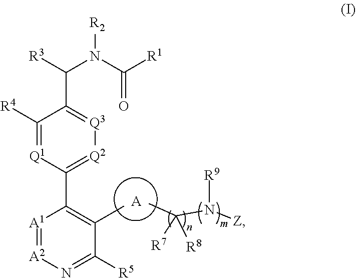
or a pharmaceutically acceptable salt thereof, wherein:
one of A¹ and A² is C—R^{6A}, and the other of A¹ and A² is C—R^{6A} or N;
Q¹ is selected from C—R⁶ and N;
Q² is selected from C—R⁶ and N;
Q³ is selected from C—R⁶ and N;
wherein at most one of Q¹, Q², and Q³ is N;
ring A is a 4- to 8-membered monocyclic saturated or partially saturated heterocyclyl, substituted with one or more R¹¹;
n is 0 or 1;
m is 0 or 1;
R¹ is selected from —N(R^{1a})₂, phenyl, 3- to 7-membered saturated or partially unsaturated monocyclic carbocyclyl, 3- to 7-membered saturated or partially unsaturated monocyclic heterocyclyl, 5- to 6-membered heteroaryl, 7- to 10-membered saturated or partially unsaturated bicyclic carbocyclyl, 7- to 10-membered saturated or partially unsaturated bicyclic heterocyclyl, 8 to 10-membered bicyclic heteroaryl, and 9- to 10-membered bicyclic aryl, wherein the phenyl, 3- to 7-membered saturated or partially unsaturated monocyclic carbocyclyl, 3- to 7-membered saturated or partially unsaturated monocyclic heterocyclyl, 5- to 6-membered heteroaryl, 7- to 10-membered saturated or partially unsaturated bicyclic carbocyclyl, 7- to 10-membered saturated or partially unsaturated bicyclic heterocyclyl, and 9- to 10-membered bicyclic aryl represented by R¹ are each optionally substituted with one or more R¹²;
R^{1a}, for each occurrence, is independently selected from H, C₁₋₆ alkyl, C₂₋₆ alkenyl, C₂₋₆ alkynyl, phenyl, 3- to 7-membered saturated or partially unsaturated carbocyclyl ring, 3- to 7-membered saturated or partially unsaturated monocyclic heterocyclyl, and 5- to 6-membered heteroaryl, wherein the C₁₋₆ alkyl, C₂₋₆ alkenyl, C₂₋₆ alkynyl, phenyl, 3- to 7-membered saturated or partially unsaturated carbocyclyl ring, 3- to 7-membered saturated or partially unsaturated monocyclic heterocyclyl, and 5- to 6-membered heteroaryl represented by R^{1a} are each optionally substituted with one or more R¹²;
or two R^{1a} groups on the same nitrogen are taken together with their intervening atoms to form a ring selected from 3- to 7-membered saturated or partially unsaturated monocyclic heterocyclyl and 5- to 6-membered heteroaryl, wherein the ring is optionally substituted with one or more R¹²;
R¹², for each occurrence, is independently selected from halogen, —OR^{12a}, —S(O)₂R^{12a}, —CN, C₁₋₆ alkyl, C₂₋₆ alkenyl, C₂₋₆ alkynyl, 3- to 7-membered saturated or partially unsaturated monocyclic carbocyclyl, and 4- to 6-membered saturated or partially unsaturated monocyclic heterocyclyl; wherein the C₁₋₆ alkyl, C₂₋₆ alkenyl, C₂₋₆ alkynyl, 3- to 7-membered saturated or partially unsaturated monocyclic carbocyclyl, and 4- to 6-membered saturated or partially unsaturated monocyclic heterocyclyl represented by R¹² are each optionally substituted with one or more R⁵;
R^{12a} is C₁₋₆ alkyl optionally substituted with one or more halogen;
R¹⁵, for each occurrence, is independently selected from halogen, C₁₋₆ alkyl, C₁₋₆ haloalkyl, —CN, and —OR^{15a};
R^{15a} is C₁₋₆ alkyl;
R² is H, C₁₋₆ alkyl, C₂₋₆ alkenyl, and C₂₋₆ alkynyl,
or R¹ and R², together with their intervening atoms, form a Ring B selected from 3- to 7-membered saturated or partially unsaturated monocyclic heterocyclyl, 7- to 10-membered saturated or partially unsaturated bicyclic heterocyclyl, and 8- to 10-membered bicyclic heteroaryl, wherein Ring B is optionally substituted with one or more R¹⁰⁰;
R¹⁰⁰, for each occurrence, is independently selected from C₁₋₆ alkyl, C₂₋₆ alkenyl, C₂₋₆ alkynyl, 3- to 7-membered saturated or partially unsaturated monocyclic carbocyclyl, 4- to 6-membered saturated or partially unsaturated monocyclic heterocyclyl and halogen; wherein the C₁₋₆ alkyl, C₂₋₆ alkenyl, C₂₋₆ alkynyl, 3- to 7-membered saturated or partially unsaturated monocyclic carbocyclyl, and saturated or partially unsaturated 4- to 6-membered monocyclic heterocyclyl represented by R¹⁰⁰ are each optionally substituted with one or more R¹⁵⁰;
R¹⁵⁰, for each occurrence, is independently selected from halogen and —OR^{150a};
R^{150a} is C₁₋₆ alkyl;
R³ is selected from H, halogen, —C(O)N(R^{3a})₂, —C(O)OR^{3a}, —C(O)R^{3a}, C₁₋₆ alkyl, C₂₋₆ alkenyl, and C₂₋₆ alkynyl, wherein the C₁₋₆ alkyl, C₂₋₆ alkenyl, and C₂₋₆ alkynyl represented by R³ are each optionally substituted with one or more substituents selected from halogen and hydroxyl;
R^{3a}, for each occurrence, is independently selected from H, C₁₋₆ alkyl, C₂₋₆ alkenyl, C₂₋₆ alkynyl, phenyl, 3- to 7-membered saturated or partially unsaturated carbocyclyl ring, 3- to 7-membered saturated or partially unsaturated monocyclic heterocyclyl, or 5- to 6-membered heteroaryl, wherein C₁₋₆ alkyl, C₂₋₆ alkenyl, C₂₋₆ alkynyl, phenyl, 3- to 7-membered saturated or partially unsaturated carbocyclyl ring, 3- to 7-membered saturated or partially unsaturated monocyclic heterocyclyl, and 5- to 6-membered heteroaryl are optionally substituted with one or more R³⁰;
or two R^{3a} groups on the same nitrogen are taken together with their intervening atoms to form a ring selected from 3- to 7-membered saturated or partially unsaturated monocyclic heterocyclyl and 5- to 6-membered heteroaryl, wherein said ring is optionally substituted with one or more R³⁰;
R³⁰, for each occurrence, is independently selected from halogen, —OR^{30a}, —N(R^{30a})₂, —C(O)N(R^{30a}), —C(O)₂R^{30a}, oxo, C₁₋₆ alkyl, C₂₋₆ alkenyl, C₂₋₆ alkynyl, 3- to 7-membered saturated or partially unsaturated monocyclic carbocyclyl, and 4- to 6-membered saturated or partially unsaturated monocyclic heterocyclyl;
R^{30a} is H or C₁₋₆ alkyl;
R⁴ is selected from H, halogen, —NO₂, —CN, —OR^{4a}, —SR^{4a}, —N(R^{4a})₂, —C(O)R^{4a}, —C(O)OR^{4a}, —S(O)R^{4a}, —S(O)₂R^{4a}, —C(O)N(R^{4a})₂, —SO₂N(R^{4a})₂, —OC(O)R^{4a}, —N(R^{4a})C(O)R^{4a}, —N(R^{4a})C(O)OR^{4a}, —N(R^{4a})SO₂R^{4a}, —OC(O)N(R^{4a})₂, C₁₋₆ alkyl, C₂₋₆ alkenyl and C₂₋₆ alkynyl, wherein the C₁₋₆ alkyl, C₂₋₆ alkenyl and C₂₋₆ alkynyl are each optionally substituted with one or more R⁴⁰;
R^{4a} is H, C₁₋₆ alkyl, C₂₋₆ alkenyl, C₂₋₆alkynyl, phenyl, 3- to 8-membered saturated or partially unsaturated carbocyclyl ring, 3- to 7-membered saturated or partially unsaturated monocyclic heterocyclyl, and 5- to 6-membered heteroaryl, wherein the C₁₋₆ alkyl, C₂₋₆ alkenyl, C₂₋₆ alkynyl, phenyl, 3- to 8-membered saturated or partially unsaturated carbocyclyl ring, 3- to 7-membered saturated or partially unsaturated monocyclic heterocyclyl, and 5- to 6-membered heteroaryl represented by R^{4a} are each optionally substituted with one or more R⁴⁰;
or two R^{4a} groups on the same nitrogen are taken together with their intervening atoms to form a ring selected from 3- to 7-membered saturated or partially unsaturated monocyclic heterocyclyl and 5- to 6-membered heteroaryl, wherein said ring is optionally substituted with one or more R⁴⁰;
R⁴⁰, for each occurrence, is independently selected from halogen, —OR^{40a}, —N(R^{40a})₂, —C(O)N(R^{40a})₂, —C(O)₂R^{40a}, oxo, C₁₋₆ alkyl, C₂₋₆ alkenyl, C₂₋₆ alkynyl, 3- to 7-membered monocyclic carbocyclyl, and 4- to 6-membered monocyclic heterocyclyl; wherein the C₁₋₆ alkyl, C₂₋₆ alkenyl, C₂₋₆ alkynyl, 3- to 7-membered monocyclic carbocyclyl, and 4- to 6-membered monocyclic heterocyclyl represented by R⁴⁰ are each optionally substituted with one or more R⁴⁵;
R^{40a} is H, C₁₋₆ alkyl, C₂₋₆ alkenyl, C₂₋₆ alkynyl, 3- to 7-membered monocyclic carbocyclyl, and 4- to 6-membered monocyclic heterocyclyl, wherein the C₁₋₆ alkyl, C₂₋₆ alkenyl, C₂₋₆ alkynyl, 3 to 7-membered monocyclic carbocyclyl, and 4- to 6-membered monocyclic heterocyclyl are each optionally substituted with one or more R⁴⁵;
R⁴⁵, for each occurrence, is independently selected from C₁₋₆ alkyl, halogen and —OR^{45a};
R^{45a} is H or C₁₋₆ alkyl;
or R³ and R⁴, together with their intervening atoms, form a Ring C, wherein Ring C is selected from 5- to 7-membered monocyclic carbocycle and 5- to 7-membered monocyclic heterocycle, wherein Ring C is optionally substituted with R³⁰⁰;
R³⁰⁰, for each occurrence, is independently selected from C₁₋₆ alkyl, C₂₋₆ alkenyl, C₂₋₆ alkynyl, 3- to 7-membered monocyclic carbocyclyl, 4- to 6-membered monocyclic heterocyclyl, halogen, —C(O)R^{300a}, —OR^{300a}, and —S(O)₂R^{300a}; wherein the C₁₋₆ alkyl, C₂₋₆ alkenyl, C₂₋₆ alkynyl, 3- to 7-membered monocyclic carbocyclyl, and 4- to 6-membered monocyclic heterocyclyl represented by R³⁰⁰ are each optionally substituted with one or more R³⁵⁰;
R^{300a} is selected from H, C₁₋₆ alkyl, C₂₋₆ alkenyl, C₂₋₆ alkynyl, 3- to 7-membered monocyclic carbocyclyl, and 4- to 6-membered monocyclic heterocyclyl, wherein the C₁₋₆ alkyl, C₂₋₆ alkenyl, C₂-6 alkynyl, 3- to 7-membered monocyclic carbocyclyl, and 4- to 6-membered monocyclic heterocyclyl represented by R^{300a} are each optionally substituted with one or more R³⁵⁰;
R³⁵⁰, for each occurrence, is independently selected from C₁₋₆ alkyl,
halogen, —CN, —C(O)R^{350a}, —C(O)N(R^{350a})₂, —C(R^{350a})₂N(R^{350a})₂, and —OR^{350a};
R^{350a}, for each occurrence, is independently H or C₁₋₆ alkyl optionally substituted with one to three halogen;
R⁵ is selected from H, —NHR^{5s}, or —NHC(O)R^{5s};
R^{5a} is H or C₁₋₆alkyl;
R⁶ and R^{6A}, for each occurrence, are independently selected from H, C₁₋₆ alkyl, C₂₋₆ alkenyl, C₂₋₆ alkynyl, halogen, —NO₂, —CN, —OR^{6a}, —SR^{6a}, —N(R^{6a})₂, —C(O)R^{6a}, —C(O)OR^{6a}, —S(O)R^{6a}, —S(O)₂R^{6a}, —C(O)N(R^{6a})₂, —SO₂N(R^{6a})₂, —OC(O)R^{6a}, —N(R^{6a})C(O)R^{6a}, —N(R^{6a})C(O)OR^{6a}, —N(R^{6a})SO₂R^{6a}, and —OC(O)N(R^{6a});
R^{6a} is H, C₁₋₆ alkyl, C₂₋₆ alkenyl, C₂₋₆ alkynyl, 3- to 7-membered monocyclic carbocyclyl, and 4- to 6-membered monocyclic heterocyclyl, wherein the C₁₋₆ alkyl, C₂₋₆ alkenyl, C₂₋₆ alkynyl, 3- to 6-membered monocyclic carbocyclyl and 4- to 6-membered monocyclic heterocyclyl represented by R^{6a} are each optionally substituted with one or more R⁶⁰;
R⁶⁰, for each occurrence, is independently selected from halogen, —OR^{60a}, C₁₋₆ alkyl, C₂₋₆ alkenyl, C₂₋₆ alkynyl, 3- to 7-membered monocyclic carbocyclyl, and 4- to 6-membered monocyclic heterocyclyl; wherein the C₁₋₆ alkyl, C₂₋₆ alkenyl, C₂₋₆ alkynyl, 3- to 7-membered monocyclic carbocyclyl and 4- to 6-membered monocyclic heterocyclyl represented by R⁶⁰ are optionally substituted with one or more R⁶⁵;
R^{60a} is H, C₁₋₆ alkyl, C₂₋₆ alkenyl, C₂₋₆ alkynyl, 3- to 7-membered monocyclic carbocyclyl, and 4- to 6-membered monocyclic heterocyclyl, wherein the C₁₋₆ alkyl, C₂₋₆ alkenyl, C₂₋₆ alkynyl, 3 to 7-membered monocyclic carbocyclyl, and 4- to 6-membered monocyclic heterocyclyl represented by R^{60a} are each optionally substituted with one or more R⁶⁵;
R⁶⁵, for each occurrence, is independently selected from C₁₋₆ alkyl, halogen and —OR^{65a};
R^{65a} is H or C₁₋₆ alkyl;
R⁷ and R⁸ are each independently H or C₁₋₆ alkyl optionally substituted with one or more substituents independently selected from halogen and C₁₋₆alkoxy;
R⁹ is H, C₁₋₆ alkyl or C₃₋₆cycloalkyl, wherein the C₁₋₆alkyl is optionally substituted with one or more substituents independently selected from halogen and C₁₋₆alkoxy and the C₃₋₆cycloalkyl is optionally substituted with one or more substituents independently selected from C₁₋₆alkyl, halogen, C₁₋₆haloalkyl and C₁₋₆alkoxy;
or when m is 1, R⁹ and one of R¹¹ on ring A together with their intervening atoms form a 4 to 7-membered monocyclic saturated or partially saturated heterocyclyl, which is optionally substituted with one or more substituents independently selected from halogen, —CN, —OH, C₁₋₆ alkyl and C₁₋₆ alkoxy;
Z is —C(═O)R¹⁰, —SO₂R¹⁰, or —CN;
R¹⁰ is C₂₋₆ alkenyl, C₂₋₆ alkynyl, C₂₋₆ alkylenyl oxide, or C₄₋₇ cycloalkenyl, wherein the C₂₋₆ alkenyl represented by R¹⁰ is optionally substituted with one or more substituents independently selected from halo, C₁₋₆ alkyl, C₁₋₆ alkoxy and —NR^{10a}R^{10b}, the C₂₋₆ alkynyl represented by R¹⁰ is optionally substituted by one or more substituents independently selected from C₁₋₆ alkyl and C₁₋₆ alkoxy, and the C₂₋₆ alkylenyl oxide represented by R¹⁰ is optionally substituted by one or more C₁₋₆ alkyl;
R^{10a} and R^{10b} are each independently H or C₁₋₃ alkyl; or R^{10a} and R^{10b} together with the nitrogen atom from they are attached form a 4- to 7-membered monocyclic saturated heterocyclyl optionally substituted with one or more substituents independently selected from halo and C₁₋₆ alkyl; and
R¹¹, for each occurrence, is independently selected from H, halogen, —CN, —OH, C₁₋₆ alkyl, C₁₋₆ haloalkyl and C₁₋₆ alkoxy, or two R¹¹ together with the same carbon atom from which they are attached form a —C(═O)— group.

2. The compound of claim 1, or a pharmaceutically acceptable salt thereof, wherein:
R¹⁵, for each occurrence, is independently selected from halogen and —OR^{15a};
R¹⁰ is C₂₋₆ alkenyl, C₂₋₆ alkynyl or C₂₋₆ alkylenyl oxide, wherein the C₂₋₆ alkenyl represented by R¹⁰ is optionally substituted with one or more substituents independently selected from C₁₋₆ alkyl, C₁₋₆ alkoxy and —NR^{10a}R^{10b}, the C₂₋₆ alkynyl represented by R¹⁰ is optionally substituted by one or more substituents independently selected from C₁₋₆ alkyl and C₁₋₆ alkoxy, and the C₂₋₆ alkylenyl oxide represented by R¹⁰ is optionally substituted by one or more C₁₋₆ alkyl;
R^{10a} and R^{10b} are each independently H or C₁₋₃ alkyl; and
R¹¹, for each occurrence, is independently selected H, halogen, —CN, —OH, C₁₋₆ alkyl and C₁₋₆ alkoxy, or two R¹¹ together with the same carbon atom from which they are attached form a —C(═O)— group.

3-5. (canceled)

6. The compound of claim 1, wherein the compound is represented by Formula (IIA) or Formula (IIB): 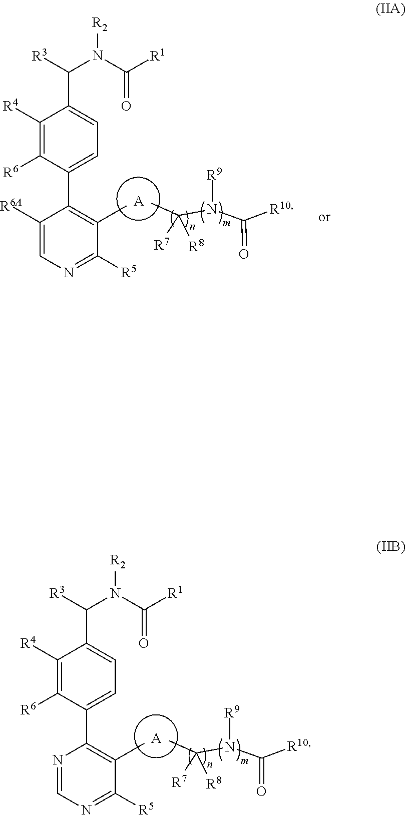
or a pharmaceutically acceptable salt thereof.

7. The compound of claim 1, or a pharmaceutically acceptable salt thereof, wherein R¹ is a 3- to 7-membered saturated or partially unsaturated monocyclic heterocyclyl having 1-2 heteroatoms independently selected from O, N and S, or a 5- to 6-membered heteroaryl having 1-4 heteroatoms independently selected from O, N and S, wherein the 3- to 7-membered saturated or partially unsaturated monocyclic heterocyclyl and 5 to 6-membered heteroaryl represented by R¹ are optionally substituted with one or two R¹².

8. (canceled)

9. The compound of claim 7, or a pharmaceutically acceptable salt thereof, wherein R¹ is a 5-membered heteroaryl selected from pyridinyl, pyrazolyl, imidazolyl, oxazolyl, isoxazolyl, 1,2,3-oxadiazolyl, 1,3,4-oxadiazolyl, 1,2,4-oxadizolyl, 1,2,3-thiadiazolyl, 1,3,4-thiadiazolyl, 1,2,4-thiadiazolyl, 1,2,3-triazolyl, 1,2,4-triazolyl, and tetrazolyl, each of which is optionally substituted with one or two R¹².

10. (canceled)

11. The compound of claim 7, or a pharmaceutically acceptable salt thereof, wherein R¹ is represented by the following formula:

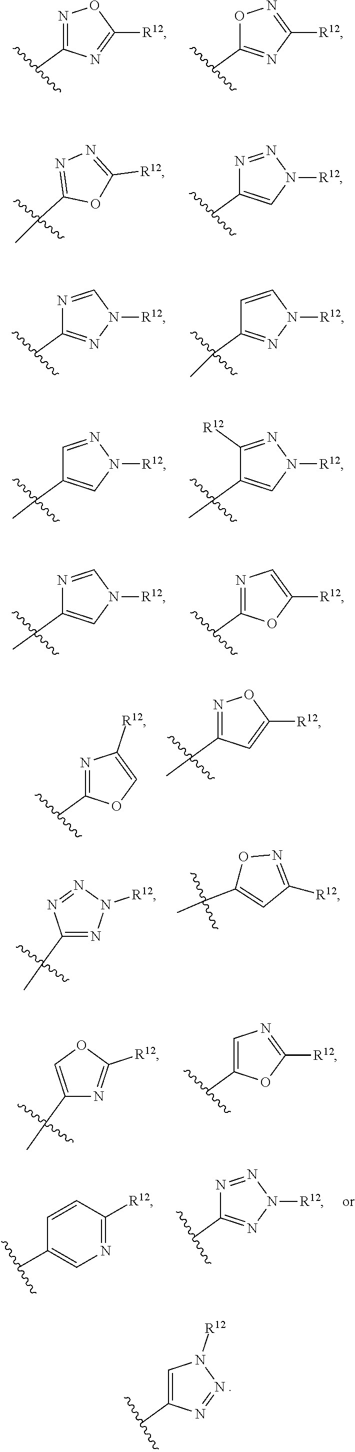

12. (canceled)

13. The compound of claim 11, or a pharmaceutically acceptable salt thereof, wherein:
R¹², for each occurrence, is independently selected from halogen, —OR^{12a}, —S(O)₂R^{12a}, —CN, C₁₋₆ alkyl, C₃₋₆ cycloalkyl, and phenyl; wherein the C₁₋₆ alkyl, C₃₋₆ cycloalkyl, and phenyl represented by R¹² are each optionally substituted with one to three R¹⁵;
R^{12a}, for each occurrence, is independently selected from H and C₁₋₃ alkyl;
R¹⁵, for each occurrence, is independently selected from C₁₋₆alkyl, halogen, C₁₋₆ haloalkyl, —CN and —OR^{15a}; and
R^{15a} is H or C₁₋₃ alkyl.

14-16. (canceled)

17. The compound of claim 1, or a pharmaceutically acceptable salt thereof, wherein R² is H or C₁₋₃ alkyl.

18. (canceled)

19. The compound of claim 1, or a pharmaceutically acceptable salt thereof, wherein R¹ and R², together with their intervening atoms, form a Ring B selected from 3- to 7-membered saturated or partially unsaturated monocyclic heterocyclyl having 1-2 heteroatoms independently selected from O, N and S, 5- to 6-membered heteroaryl having 1-4 heteroatoms independently selected from O, N and S, 7- to 10-membered bicyclic heterocyclyl having 1-4 heteroatoms independently selected from O, N and S, and 8- to 10-membered bicyclic heteroaryl having 1-4 heteroatoms independently selected from O, N and S, wherein Ring B is optionally substituted with one or two R¹⁰⁰, or wherein Ring B is represented by one of following formulae: 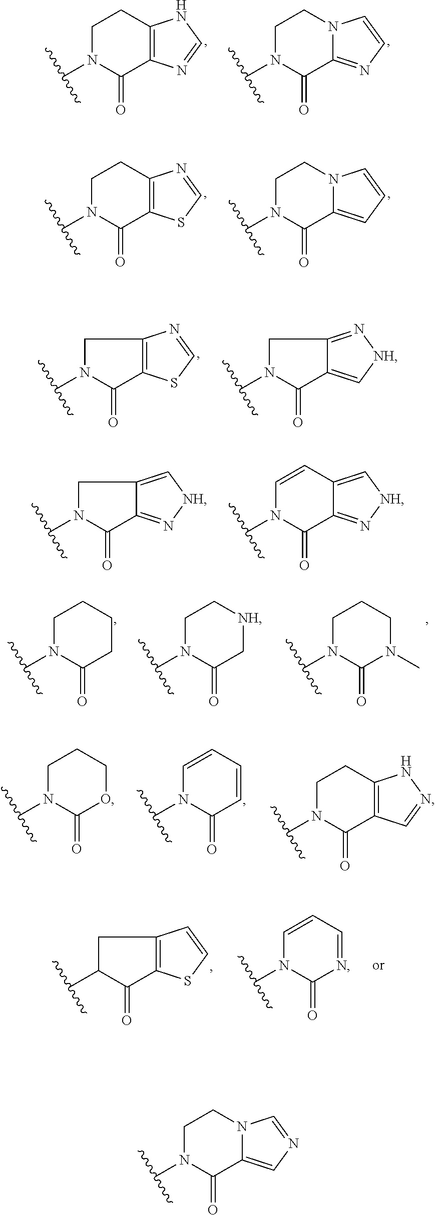
wherein Ring B is optionally substituted with one or two R¹⁰⁰, wherein:
R¹⁰⁰, for each occurrence, is independently selected from C₁₋₆ alkyl, C₃₋₆ cycloalkyl, halogen, —CN, and —OR^{100a}; wherein the C₁₋₆ alkyl and C₃₋₆ cycloalkyl are each optionally substituted with one to three substituents independently selected from halogen and C₁₋₃ alkyl; R^{100a}, for each occurrence, is independently selected from H, C₁₋₆ alkyl, C₃₋₆ cycloalkyl, and 4- to 6-membered monocyclic heterocyclyl.

20-23. (canceled)

24. The compound of claim 1, or a pharmaceutically acceptable salt thereof, wherein R³ is H, and R⁴ is selected from H, halogen, —CN, —OR^{4a}, C₁₋₆ alkyl, and C₃₋₆ cycloalkyl, wherein the C₁₋₆ alkyl and C₃₋₆ cycloalkyl represented by R⁴ are each optionally substituted with one to three halogen; and
R^{4a} is C₁₋₄ alkyl optionally substituted with one to three halogen.

25-27. (canceled)

28. The compound of claim 1, or a pharmaceutically acceptable salt thereof, wherein R³ and R⁴, together with their intervening atoms, form a Ring C, wherein Ring C is selected from 5- to 7-membered monocyclic carbocycle and 5- to 7-membered monocyclic heterocycle, wherein Ring C is optionally substituted with R³⁰⁰.

29. The compound of claim 1, or a pharmaceutically acceptable salt thereof, wherein R⁶ is H or halogen; R^{6A} is H, halogen or CN; R⁵ is H or —NHR^{5a}; and R^{5a} is H or C₁₋₃ alkyl.

30-33. (canceled)

34. The compound of claim 1, or a pharmaceutically acceptable salt thereof, wherein ring A is a 4- to 8-membered monocyclic saturated azacyclic ring, optionally substituted with one or two R¹¹; m is 0 or 1; n is 0; and R⁹ and one R¹¹ together with intervening atoms form a 4- to 8-membered monocyclic saturated azacyclic ring.

35-37. (canceled)

38. The compound of claim 1, wherein the compound is represented by the following formula: 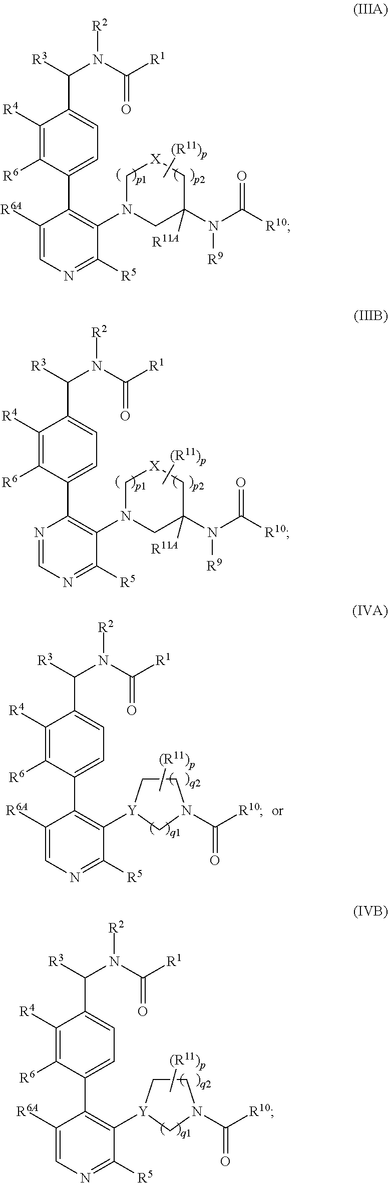 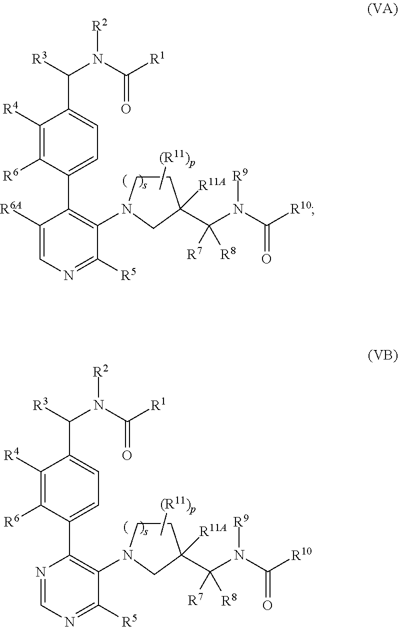
or a pharmaceutically acceptable salt thereof, wherein:
X is O or CHR^{11B};
R^{11A} is H; or R^{11A} and R⁹ together with their intervening atoms form a 4- to 6-membered saturated monocyclic azacyclic ring;
R^{11B} is H; or R^{11B} and R⁹ together with their intervening atoms form a 4- to 6-membered saturated monocyclic azacyclic ring;
Y is CH or N;
p is 0, 1, 2, 3 or 4;
p1 is 0, 1, or 2;
p2 is 0, 1 or 2;
q1 is 0, 1, or 2, provided when Y is N, q1 is not 0;
q2 is 0, 1 or 2; provided that q1 and q2 cannot both be 0; and
s is 0, 1, or 2.

39. (canceled)

40. The compound of claim 38, wherein the compound is represented by the following formula: 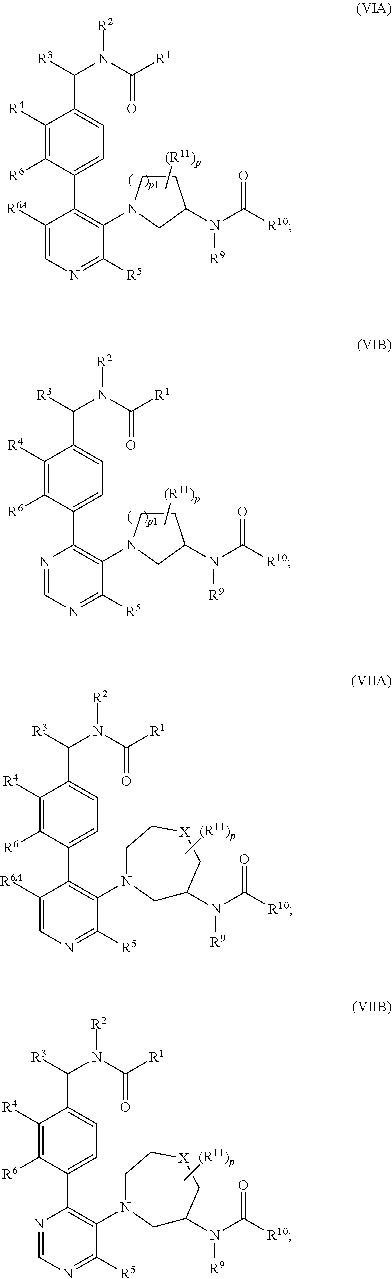 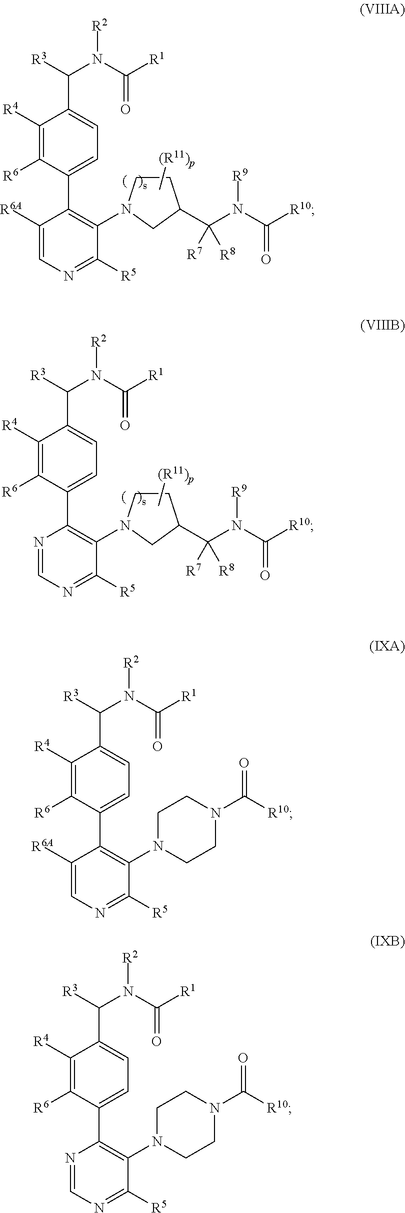 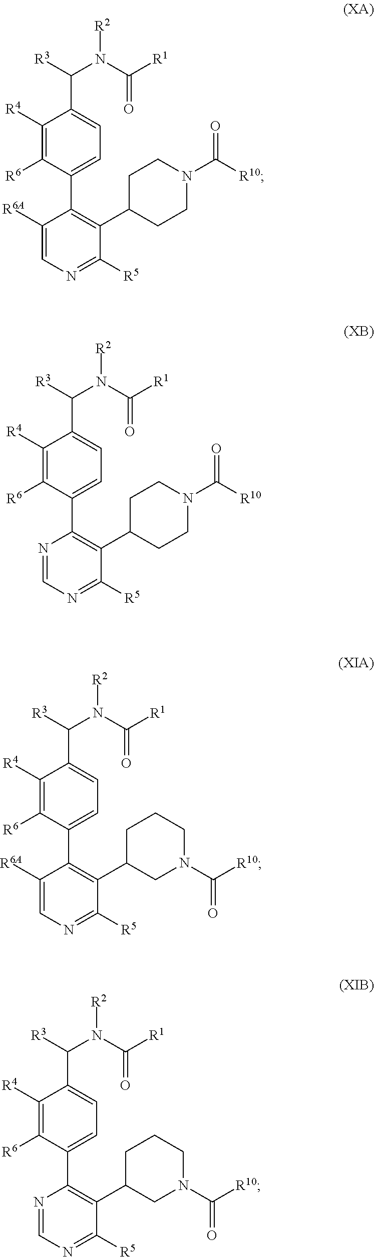 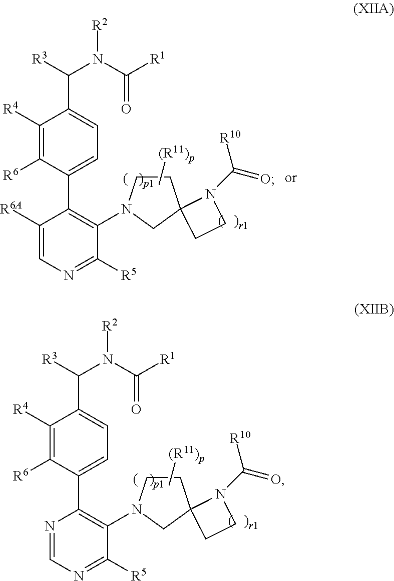
or a pharmaceutically acceptable salt thereof, wherein:
p1 is 1 or 2;
s is 1 or 2;
r1 is 1 or 2; and
X is O or CH₂ and optionally wherein p is 2 and two R¹¹ together with the same carbon atom from which they are attached form a —C(═O)— group.

41-43. (canceled)

44. The compound of claim 1, or a pharmaceutically acceptable salt thereof, wherein R⁹ is C₁₋₃alkyl or C₃₋₆cycloalkyl; R¹ is C₂₋₆ alkenyl or C₄₋₇ cycloalkenyl optionally substituted with one or more halo, C₁₋₆ alkyl, C₁₋₆ alkoxy or —NR^{10a}R^{10b}, and R^{10a} and R^{10b} are each independently H or C₁₋₃ alkyl, or R^{10a} and R^{10b} together with the nitrogen atom from which they are attached form a 4- to 7-membered monocyclic saturated heterocyclyl optionally substituted with one or more substituents independently selected from halo and C₁₋₆ alkyl.

45-50. (canceled)

51. The compound of claim 38, or a pharmaceutically acceptable salt thereof, wherein:
R¹ is represented by the following formula: 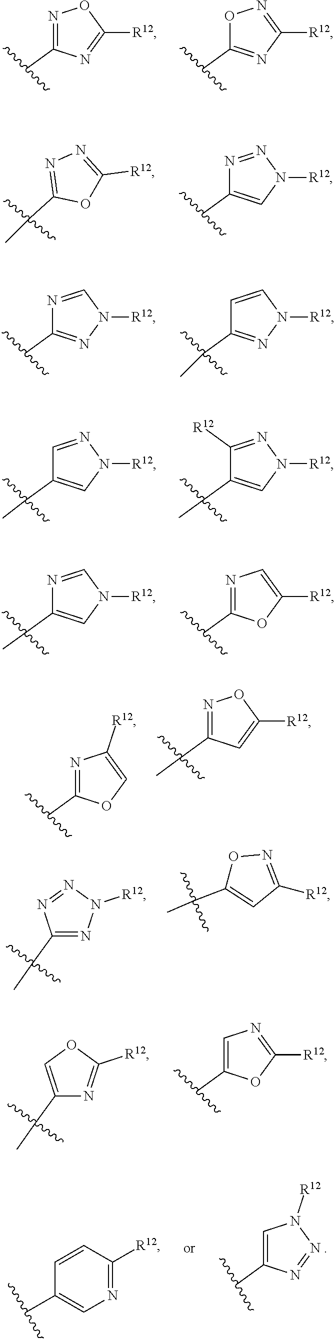
R¹², for each occurrence, is independently C₁₋₄ alkyl optionally substituted with one to three halogen or a C₃₋₆cycloalkyl optionally substituted with one or two C₁₋₃alkyl;
R² is H or C₁₋₃alkyl;
R³ is H;
R⁴ is C₁₋₃alkyl;
R⁵ is H;
R⁶ is H or halogen; and
R^{6A} is H, halogen or CN.

52-53. (canceled)

54. A pharmaceutical composition comprising a compound of claim 1 or a pharmaceutically acceptable salt thereof, and a pharmaceutically acceptable excipient.

55. A method of treating a disorder responsive to inhibition of Bruton's tyrosine kinase in a subject comprising administering to the subject an effective amount of the compound according to claim 1, or a pharmaceutically acceptable salt thereof.

56. The method of claim 55, wherein the disorder is an autoimmune disorder; rheumatoid arthritis, lupus erythematosus, atopic dermatitis, leukemia or lymphoma; or optionally multiple sclerosis.

57-61. (canceled)
